(19) United States
(12) Patent Application Publication
(10) Pub. No.: US 2024/0327867 A1
(43) Pub. Date: Oct. 3, 2024

(54) ANELLOVIRUS CONSTRUCTS COMPRISING RECOMBINATION SITES AND CROSS-PACKAGING OF ANELLOVECTOR PARTICLES AND GENETIC ELEMENTS

(71) Applicant: FLAGSHIP PIONEERING INNOVATIONS V, INC., Cambridge, MA (US)

(72) Inventors: Joseph Michael Cabral, East Boston, MA (US); Cato Hawkins Prince, Waltham, MA (US); Bryan Alexander McGuffie, Jamaica Plain, MA (US); Hassana Kia Oyibo, Everett, MA (US); Simon Delagrave, Sudbury, MA (US); Dhananjay Maniklal Nawandar, Waltham, MA (US); Kevin James Lebo, Weymouth, MA (US); Cesar Augusto Arze, Boston, MA (US); Agamoni Bhattacharyya, Allston, MA (US); Nathan Lawrence Yozwiak, Newton, MA (US); Roger Joseph Hajjar, Lexington, MA (US); Geoffrey Parsons, Jamaica Plain, MA (US)

(21) Appl. No.: 18/605,384

(22) Filed: Mar. 14, 2024

Related U.S. Application Data
(60) Provisional application No. 63/505,515, filed on Jun. 1, 2023.
(60) Provisional application No. 63/490,457, filed on Mar. 15, 2023.

(51) Int. Cl. C12N 15/86; A61K 47/64; C07K 14/005

(52) U.S. Cl. CPC C12N 15/86; A61K 47/64; C07K 14/005; C12N 2750/00022; C12N 2750/00023; C12N 2750/00043; C12N 2750/00051; C12N 2750/00071; C12N 2830/008; C12N 2840/203

(57) ABSTRACT

This invention relates generally to modified anellovirus capsid proteins, anellovectors, anelloVLPs, and compositions and uses thereof.

### CROSS REFERENCE TO RELATED APPLICATIONS

This application claims the benefit of U.S. Provisional Application No. 63/490,457, filed Mar. 15, 2023, and U.S. Provisional Application No. 63/505,515, filed Jun. 1, 2023. The contents of the aforementioned applications are hereby incorporated by reference in their entirety.

### SEQUENCE LISTING

The instant application contains a Sequence Listing which has been submitted electronically in XML file format and is hereby incorporated by reference in its entirety. Said XML copy, created on Mar. 13, 2024, is named V2057-701310_SL.xml and is 1,773,093 bytes in size.

### BACKGROUND

There is an ongoing need to develop suitable vectors to deliver therapeutic agents to patients.

### SUMMARY

The present disclosure provides an Anelloviridae family vector (e.g., anellovector), e.g., a synthetic Anelloviridae family vector (e.g., anellovector), that can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). In some embodiments, an Anelloviridae family vector (e.g., particle, e.g., a viral particle, e.g., an Anellovirus particle) comprises a genetic element (e.g., a genetic element comprising a therapeutic DNA sequence) encapsulated in a proteinaceous exterior (e.g., a proteinaceous exterior comprising an Anelloviridae family viral capsid protein, e.g., an Anellovirus ORF1 molecule or a polypeptide encoded by an Anellovirus ORF1 nucleic acid, or a chicken anemia virus (CAV) VP1 molecule or a polypeptide encoded by a CAV VP1 nucleic acid, e.g., as described herein), which is capable of introducing the genetic element into a cell (e.g., a mammalian cell, e.g., a human cell). In some embodiments, the Anelloviridae family vector is a particle comprising a proteinaceous exterior comprising a polypeptide encoded by an Anelloviridae family viral protein, e.g., an Anellovirus ORF1 nucleic acid (e.g., an ORF1 nucleic acid of an Alphatorquevirus, Betatorquevirus, or Gammatorquevirus, e.g., as described herein) or a CAV VP1 nucleic acid.

It is understood that embodiments described herein with respect to anellovectors, anelloVLPs, or Anelloviruses generally can also be applicable to other Anelloviridae family viruses (e.g., CAV), or vectors and VLPs based thereon. For example, methods and compositions described herein relating to anellovectors can be used for vectorized particles comprising components (e.g., polypeptide or nucleic acid sequences) from CAV or other Anelloviridae family viruses. In some instances, methods and compositions described herein relating to anelloVLPs can be used for virus-like particles (VLPs) comprising components (e.g., polypeptide or nucleic acid sequences) from CAV or other Anelloviridae family viruses. In some instances, methods and compositions described herein relating to Anelloviruses can be applied to corresponding components (e.g., polypeptide or nucleic acid molecules) from CAV or other Anelloviridae family viruses. In some instances, methods or compositions utilizing or producing an Anellovirus ORF molecule (e.g., an Anellovirus ORF1, ORF2, or ORF3 molecule), or a nucleic acid encoding same, can be applied to polypeptides from other Anelloviridae family viruses (e.g., a CAV VP1, VP2, or VP3 molecule), or nucleic acids encoding same.

In some instances, a description herein of a nucleic acid sequence (e.g., an Anelloviridae family viral sequence, or an element or fragment thereof; an Anelloviridae family vector sequence, or an element or fragment thereof; a recombinase recognition site; or a recombinase hybrid site) can refer to the sense strand, the antisense strand, or a double-stranded DNA comprising both the sense strand and antisense strands, or any sequence thereof. In some instances, a listing of a nucleic acid sequence herein can refer to the sequence listed and/or its reverse complement. In some embodiments, the proteinaceous exterior of an anellovector or anelloVLP comprises a modified Anellovirus ORF1 molecule. In some embodiments, the Anellovirus ORF1 molecule is modified to delete at least a portion of the structural arginine-rich region (e.g., as described herein). In some embodiments, the Anellovirus ORF1 molecule is modified to delete at least a portion of the structural C-terminal domain (e.g., as described herein). In some embodiments, the Anellovirus ORF1 molecule is a chimeric ORF1 molecule comprising a fragment or domain (e.g., a structural arginine-rich region, a P1 domain, a P2 domain, a P1-1 domain, and/or a P1-2 domain, e.g., as described herein) from a different Anellovirus ORF1 protein (e.g., as described herein). In some embodiments, the Anellovirus ORF1 molecule is a chimeric ORF1 molecule comprising a fragment or domain from a protein other than an Anellovirus ORF1 protein (e.g., a protein from another virus, e.g., as described herein).

In some embodiments, the anellovector or anelloVLP comprises on its exterior surface (e.g., attached to a proteinaceous exterior) a surface moiety as described herein. In some embodiments, the proteinaceous exterior comprises an ORF1 molecule attached to the surface moiety. In some embodiments, the proteinaceous exterior comprises an ORF1 molecule comprising a click handle. In some embodiments, the proteinaceous exterior comprises an ORF1 molecule fused to a polypeptide surface moiety. In some embodiments, the proteinaceous exterior comprises a plurality of ORF1 molecules each attached to a surface moiety, e.g., wherein the plurality of ORF1 molecules form a multimer (e.g., a dimer, trimer, or pentamer).

The genetic element of an anellovector of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some instances, the genetic element is circular or linear. In some instances, the genetic element comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell. In another example, the anellovector can deliver and express an effector, e.g., an exogenous protein, in vivo. Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject. In some instances, the anellovector is made by in vitro assembly. In vitro assembly of an anellovector generally involves the formation of a proteinaceous exterior enclosing a genetic element, which occurs outside of a host cell (e.g., in a cell-free suspension, lysate, or supernatant). In vitro assembly may, in some instances, utilize components generated in a host cell but does not generally require a host cell for particle assembly.

The present disclosure provides an anelloVLP, e.g., a synthetic anelloVLP, which can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). The anelloVLP generally comprises on its exterior surface (e.g., attached to a proteinaceous exterior) a surface moiety as described herein. In some embodiments, the surface moiety comprises the effector. In some embodiments, the surface moiety comprises a targeting agent (e.g., an agent that targets the anelloVLP to a target cell or tissue). In some embodiments, an anelloVLP (e.g., particle, e.g., a viral particle, e.g., an Anellovirus particle) comprises a proteinaceous exterior (e.g., a proteinaceous exterior comprising an Anellovirus capsid protein, e.g., an Anellovirus ORF1 molecule or a polypeptide encoded by an Anellovirus ORF1 nucleic acid, e.g., as described herein). In some embodiments, the anelloVLP is a particle comprising a proteinaceous exterior comprising a polypeptide encoded by an Anellovirus ORF1 nucleic acid (e.g., an ORF1 nucleic acid of Betatorquevirus, e.g., as described herein). In some embodiments, the proteinaceous exterior encloses an effector. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the anelloVLP can deliver an effector into a cell by contacting the cell and introducing the effector into the cell. In certain instances, the effector is an endogenous effector (e.g., endogenous to the target cell but, e.g., provided in increased amounts by the anelloVLP). In other instances, the effector is an exogenous effector. The effector can, in some instances, modulate a function of the cell or modulate an activity or level of a target molecule in the cell. For example, the effector can decrease levels of a target protein in the cell. In another example, the anelloVLP can deliver an effector, e.g., an exogenous protein, in vivo. AnelloVLPs can be used, for example, to deliver an effector to a target cell, tissue or subject; or for treatment of diseases and disorders, e.g., by delivering an effector that can operate as a therapeutic agent to a desired cell, tissue, or subject. In some instances, the anelloVLP is made by in vitro assembly. In vitro assembly of an anelloVLP generally involves the formation of a proteinaceous exterior in connection with an effector (e.g., the proteinaceous exterior enclosing the effector), which occurs outside of a host cell (e.g., in a cell-free suspension, lysate, or supernatant). In vitro assembly of an anelloVLP may, in some instances, utilize components generated in a host cell but does not generally require a host cell for particle assembly.

The invention further provides synthetic anellovectors and synthetic anelloVLPs. A synthetic anellovector or synthetic anelloVLP has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors and synthetic anelloVLPs include a proteinaceous exterior, which can be used for delivering an effector (e.g., an exogenous effector or an endogenous effector) into eukaryotic (e.g., human) cells. In some embodiments, the anellovector or anelloVLP does not cause a detectable and/or an unwanted immune or inflammatory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector or anelloVLP may be substantially non-immunogenic to the target cell, tissue or subject.

In an aspect, the invention features an anellovector comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the anellovector is capable of delivering the genetic element into a eukaryotic (e.g., mammalian, e.g., human) cell. In some embodiments, the anellovector comprises a surface moiety (e.g., a surface moiety having effector and/or targeting function), e.g., displayed on the exterior surface of the anellovector (e.g., as described herein). In some embodiments, the surface moiety comprises the effector.

In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined as described in Wang et al. (2004, Gene Therapy 11: 711-721, incorporated herein by reference in its entirety). In some embodiments, the genetic element is enclosed within the proteinaceous exterior. In some embodiments, the anellovector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as listed in any one of Tables A1-A32 or N1-N32). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Anellovirus sequence as described herein, e.g., as listed in any one of Tables A1-A32 or N1-N32). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In an aspect, the invention features an anelloVLP comprising a proteinaceous exterior (e.g., a capsid) and an effector; wherein the anelloVLP is capable of delivering the effector into a eukaryotic (e.g., mammalian, e.g., human) cell. In some embodiments, the effector is comprised in a surface moiety, e.g., displayed on the exterior surface of the anelloVLP (e.g., as described herein).

In an aspect, the invention features an infectious (to a human cell) particle comprising an Anellovirus capsid (e.g., a capsid comprising an Anellovirus ORF, e.g., ORF1, polypeptide). In some embodiments, the infectious particle encapsulates a genetic element comprising a protein binding sequence that binds to the capsid and a heterologous (to the Anellovirus) sequence encoding a therapeutic effector. In some embodiments, the particle is capable of delivering the genetic element into a mammalian, e.g., human, cell. In some embodiments, the genetic element has less than about 6% (e.g., less than 6%, 5.5%, 5%, 4.5%, 4%, 3.5%, 3%, 2.5%, 2%, 1.5%, or less) identity to a wild type Anellovirus. In some embodiments, the genetic element has no more than 1.5%, 2%, 2.5%, 3%, 3.5%, 4%, 4.5%, 5%, 5.5% or 6% identity to a wild type Anellovirus. In some embodiments, the genetic element has at least about 2% to at least about 5.5% (e.g., 2 to 5%, 3% to 5%, 4% to 5%) identity to a wild type Anellovirus. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety).

Also described herein are viral vectors and viral particles based on Anelloviruses, which can be used to deliver an agent (e.g., an exogenous effector or an endogenous effector, e.g., a therapeutic effector) to a cell (e.g., a cell in a subject to be treated therapeutically). In some embodiments, Anelloviruses can be used as effective delivery vehicles for introducing an agent, such as an effector described herein, to a target cell, e.g., a target cell in a subject to be treated therapeutically or prophylactically.

In an aspect, the invention features a polypeptide (e.g., a synthetic polypeptide, e.g., an ORF1 molecule) comprising (e.g., in series):
(i) a first region comprising a structural arginine-rich region, e.g., amino acid sequence having at least 70% (e.g., at least about 70, 80, 90, 95, 96, 97, 98, 99, or 100%) sequence identity to a structural arginine-rich region sequence described herein or a sequence of at least about 40 amino acids comprising at least 60%, 70%, or 80% basic residues (e.g., arginine, lysine, or a combination thereof),
(ii) a second region comprising a structural jelly-roll domain, e.g., an amino acid sequence having at least 30% (e.g., at least about 30, 35, 40, 50, 60, 70, 80, 90, 95, 96, 97, 98, 99, or 100%) sequence identity to a structural jelly-roll region sequence described herein or a sequence comprising at least 6 beta strands,
(iii) a third region comprising an amino acid sequence having at least 30% (e.g., at least about 30, 35, 40, 50, 60, 70, 80, 90, 95, 96, 97, 98, 99, or 100%) sequence identity to an structural N22 domain sequence described herein,
(iv) a fourth region comprising an amino acid sequence having at least 70% (e.g., at least about 70, 80, 90, 95, 96, 97, 98, 99, or 100%) sequence identity to an Anellovirus ORF1 structural C-terminal domain (CTD) sequence described herein, and
(v) optionally wherein the polypeptide has an amino acid sequence having less than 100%, 99%, 98%, 95%, 90%, 85%, 80% sequence identity to a wild type Anellovirus ORF1 protein described herein.

In some embodiments, the polypeptide comprises at least about 70, 80, 90, 95, 96, 97, 98, 99, or 100% sequence identity to an Anellovirus ORF1 molecule as described herein (e.g., as listed in any one of Tables A1-A32). In some embodiments, the polypeptide comprises at least about 70, 80, 90, 95, 96, 97, 98, 99, or 100% sequence identity to a subsequence (e.g., a structural arginine (Arg)-rich domain, a structural jelly-roll domain, a hypervariable region (HVR), an structural N22 domain, or a structural C-terminal domain (CTD)) of an Anellovirus ORF1 molecule as described herein. In one embodiment, the amino acid sequences of the (i), (ii), (iii), and (iv) region have at least 90% sequence identity to their respective references and wherein the polypeptide has an amino acid sequence having less than 100%, 99%, 98%, 95%, 90%, 85%, 80% sequence identity to a wild type Anellovirus ORF1 protein described herein.

In an aspect, the invention features a complex comprising a polypeptide as described herein (e.g., an Anellovirus ORF1 molecule as described herein) and a genetic element comprising a promoter element and a nucleic acid sequence (e.g., a DNA sequence) encoding an effector (e.g., an exogenous effector or an endogenous effector), and a protein binding sequence.

The present disclosure further provides nucleic acid molecules (e.g., a nucleic acid molecule that includes a genetic element as described herein, or a nucleic acid molecule that includes a sequence encoding a proteinaceous exterior protein as described herein). A nucleic acid molecule of the invention may include one or both of (a) a genetic element as described herein, and (b) a nucleic acid sequence encoding a proteinaceous exterior protein as described herein.

In an aspect, the invention features an isolated nucleic acid molecule comprising a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus, as disclosed herein. In some embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2. In embodiments, the promoter element is capable of directing expression of the effector in a eukaryotic (e.g., mammalian, e.g., human) cell.

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, the genetic element comprises an anellovector, e.g., as described herein. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein).

In an aspect, the invention features a host cell or helper cell comprising: (a) a nucleic acid comprising a sequence encoding one or more of an ORF1 molecule, an ORF2 molecule, or an ORF3 molecule (e.g., a sequence encoding an Anellovirus ORF1 polypeptide described herein), wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 polypeptide (e.g., an ORF1 protein). For example, the host cell or helper cell comprises (a) and (b) either in cis (both part of the same nucleic acid molecule) or in trans (each part of a different nucleic acid molecule). In embodiments, the genetic element of (b) is circular, single-stranded DNA. In some embodiments, the host cell is a manufacturing cell line. In some embodiments, the host cell or helper cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an anellovector (e.g., a synthetic anellovector) as described herein. In embodiments, the pharmaceutical composition further comprises a pharmaceutically acceptable carrier or excipient. In embodiments, the pharmaceutical composition comprises a unit dose comprising about 10⁵-10¹⁴ genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In embodiments, the payload is a nucleic acid. In embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

In an aspect, the invention features a method of treating a disease or disorder in a subject, the method comprising administering to the subject an anelloVLP, e.g., a synthetic anelloVLP, e.g., as described herein.

In an aspect, the invention features a method of delivering an effector or payload (e.g., an endogenous or exogenous effector) to a cell, tissue or subject, the method comprising administering to the subject an anelloVLP, e.g., a synthetic anelloVLP, e.g., as described herein, wherein the anelloVLP comprises the effector (e.g., wherein the proteinaceous exterior of the anelloVLP encapsulates the effector). In embodiments, the payload is a nucleic acid. In embodiments, the payload is a polypeptide (e.g., a protein).

In an aspect, the invention features a method of delivering an anelloVLP to a cell, comprising contacting the anelloVLP, e.g., a synthetic anelloVLP, e.g., as described herein, with a cell, e.g., a eukaryotic cell, e.g., a mammalian cell, e.g., in vivo or ex vivo.

In an aspect, the invention features a method of making an anellovector, e.g., a synthetic anellovector. The method includes:
a) providing a host cell comprising:
(i) a first nucleic acid molecule comprising the nucleic acid sequence of a genetic element of an anellovector, e.g., a synthetic anellovector, as described herein, and
(ii) the first nucleic acid or a second nucleic acid molecule encoding one or more of an amino acid sequence chosen from ORF1, ORF2, ORF2/2, ORF2/3, ORF1/1, or ORF1/2, e.g., as listed in any one of Tables A1-A32, or an amino acid sequence having at least 70% (e.g., at least 70%, 75%, 80%, 85%, 90%, 95%, 96%, 97%, 98%, 99%, or 100%) sequence identity thereto; and
b) incubating the host cell under conditions suitable to make the anellovector.

In some embodiments, the method further includes, prior to step (a), introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is a helper (e.g., a helper plasmid or the genome of a helper virus).

In another aspect, the invention features a method of manufacturing an anellovector composition, comprising:
a) providing a host cell comprising, e.g., expressing one or more components (e.g., all of the components) of an anellovector, e.g., a synthetic anellovector, e.g., as described herein. For example, the host cell comprises (a) a nucleic acid comprising a sequence encoding an Anellovirus ORF1 polypeptide described herein, wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g., packaging sequence) that binds the polypeptide of (a), wherein the host cell or helper cell comprises (a) and (b) either in cis or in trans. In embodiments, the genetic element of (b) is circular, single-stranded DNA. In some embodiments, the host cell is a manufacturing cell line;
b) culturing the host cell under conditions suitable for producing a preparation of anellovectors from the host cell, wherein the anellovectors of the preparation comprise a proteinaceous exterior (e.g., comprising an ORF1 molecule) encapsulating the genetic element (e.g., as described herein), thereby making a preparation of anellovectors; and
optionally, c) formulating the preparation of anellovectors, e.g., as a pharmaceutical composition suitable for administration to a subject.

In some embodiments, the components of the anellovector are introduced into the host cell at the time of production (e.g., by transient transfection). In some embodiments, the host cell stably expresses the components of the anellovector (e.g., wherein one or more nucleic acids encoding the components of the anellovector are introduced into the host cell, or a progenitor thereof, e.g., by stable transfection).

In some embodiments, the method further comprises one or more purification steps (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

In an aspect, the invention features a method of manufacturing an anellovector composition, comprising: a) providing a plurality of anellovectors described herein, or a preparation of anellovectors described herein; and b) formulating the anellovectors or preparation thereof, e.g., as a pharmaceutical composition suitable for administration to a subject.

In an aspect, the invention features a method of manufacturing an anelloVLP composition, comprising: a) providing a plurality of anelloVLPs described herein, or a preparation of anelloVLPs described herein; and b) formulating the anelloVLPs or preparation thereof, e.g., as a pharmaceutical composition suitable for administration to a subject.

In an aspect, the invention features a method of making a host cell, e.g., a first host cell or a producer cell (e.g., as shown in FIG. 8), e.g., a population of first host cells, comprising an anellovector, the method comprising introducing a genetic element, e.g., as described herein, to a host cell and culturing the host cell under conditions suitable for production of the anellovector. In some embodiments, the method further comprises introducing a helper, e.g., a helper virus, to the host cell. In some embodiments, the introducing comprises transfection (e.g., chemical transfection) or electroporation of the host cell with the anellovector.

In an aspect, the invention features a method of making an anellovector, comprising providing a host cell, e.g., a first host cell or producer cell (e.g., as shown in FIG. 8), comprising an anellovector, e.g., as described herein, and purifying the anellovector from the host cell. In some embodiments, the method further comprises, prior to the providing step, contacting the host cell with an anellovector, e.g., as described herein, and incubating the host cell under conditions suitable for production of the anellovector. In some embodiments, the host cell is the first host cell or producer cell described in the above method of making a host cell. In some embodiments, purifying the anellovector from the host cell comprises lysing the host cell.

In some embodiments, the method further comprises a second step of contacting the anellovector produced by the first host cell or producer cell with a second host cell, e.g., a permissive cell (e.g., as shown in FIG. 8), e.g., a population of second host cells. In some embodiments, the method further comprises incubating the second host cell inder conditions suitable for production of the anellovector. In some embodiments, the method further comprises purifying an anellovector from the second host cell, e.g., thereby producing an anellovector seed population. In some embodiments, at least about 2-100-fold more of the anellovector is produced from the population of second host cells than from the population of first host cells. In some embodiments, purifying the anellovector from the second host cell comprises lysing the second host cell. In some embodiments, the method further comprises a second step of contacting the anellovector produced by the second host cell with a third host cell, e.g., permissive cells (e.g., as shown in FIG. 8), e.g., a population of third host cells. In some embodiments, the method further comprises incubating the third host cell inder conditions suitable for production of the anellovector. In some embodiments, the method further comprises purifying an anellovector from the third host cell, e.g., thereby producing an anellovector stock population. In some embodiments, purifying the anellovector from the third host cell comprises lysing the third host cell. In some embodiments, at least about 2-100-fold more of the anellovector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of anellovectors by the host cell. In some embodiments, anellovectors produced by a host cell separated from the host cell (e.g., by lysing the host cell) prior to contact with a second host cell. In some embodiments, anellovectors produced by a host cell are contacted with a second host cell without an intervening purification step.

In an aspect, the invention features a method of making a pharmaceutical anellovector preparation. The method comprises (a) making an anellovector preparation as described herein, (b) evaluating the preparation (e.g., a pharmaceutical anellovector preparation, anellovector seed population or the anellovector stock population) for one or more pharmaceutical quality control parameters, e.g., identity, purity, titer, potency (e.g., in genomic equivalents per anellovector particle), and/or the nucleic acid sequence, e.g., from the genetic element comprised by the anellovector, and (c) formulating the preparation for pharmaceutical use of the evaluation meets a predetermined criterion, e.g., meets a pharmaceutical specification. In some embodiments, evaluating identity comprises evaluating (e.g., confirming) the sequence of the genetic element of the anellovector, e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates. In some embodiments, evalating titer comprises evaluating the ratio of functional versus non-functional (e.g., infectious vs non-infectious) anellovectors in the preparation (e.g., as evaluated by HPLC). In some embodiments, evaluating potency comprises evaluating the level of anellovector function (e.g., expression and/or function of an effector encoded therein or genomic equivalents) detectable in the preparation. In some embodiments, the impurities comprise residual denaturant (e.g., urea) or cellular substituents (e.g., proteasomes or ferritin).

In some embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anellovectors can be produced in a single batch. In some embodiments, the levels of the anellovectors produced in the batch can be evaluated (e.g., individually or together).

In an aspect, the invention features a method of making a pharmaceutical anelloVLP preparation. The method comprises (a) making an anelloVLP preparation as described herein, (b) evaluating the preparation (e.g., a pharmaceutical anelloVLP preparation, anelloVLP seed population or the anelloVLP stock population) for one or more pharmaceutical quality control parameters, e.g., identity, purity, titer, potency, and (c) formulating the preparation for pharmaceutical use of the evaluation meets a predetermined criterion, e.g., meets a pharmaceutical specification. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted VLPs (e.g., an anelloVLP other than the desired anelloVLP, e.g., a synthetic anelloVLP as described herein), free viral capsid protein, adventitious agents, and aggregates. In some embodiments, evalating titer comprises evaluating the ratio of functional versus non-functional (e.g., infectious vs non-infectious) anelloVLPs in the preparation (e.g., as evaluated by HPLC). In some embodiments, evaluating potency comprises evaluating the level of anelloVLP function (e.g., expression and/or function of an effector encoded therein or genomic equivalents) detectable in the preparation. In some embodiments, the impurities comprise residual denaturant (e.g., urea) or cellular substituents (e.g., proteasomes or ferritin).

In some embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anelloVLPs can be produced in a single batch. In some embodiments, the levels of the anelloVLPs produced in the batch can be evaluated (e.g., individually or together).

In an aspect, the invention features a host cell comprising:
(i) a first nucleic acid molecule comprising the nucleic acid sequence of a genetic element of an anellovector as described herein, and
(ii) optionally, a second nucleic acid molecule encoding one or more of an amino acid sequence chosen from ORF1, ORF2, ORF2/2, ORF2/3, ORF1/1, or ORF1/2 as listed in any one of Tables A1-A32, or an amino acid sequence having at least about 70% (e.g., at least about 70, 80, 90, 95, 96, 97, 98, 99, or 100%) sequence identity thereto.

In an aspect, the invention features a reaction mixture comprising an anellovector described herein and a helper virus, wherein the helper virus comprises a polynucleotide, e.g., a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an anellovector (e.g., a synthetic anellovector) is isolated, e.g., isolated from a host cell and/or isolated from other constituents in a solution (e.g., a supernatant). In some embodiments, an anellovector (e.g., a synthetic anellovector) is purified, e.g., from a solution (e.g., a supernatant). In some embodiments, an anellovector is enriched in a solution relative to other constituents in the solution.

In some embodiments of any of the aforesaid anellovectors, compositions or methods, providing an anellovector comprises separating (e.g., harvesting) an anellovector from a composition comprising an anellovector-producing cell, e.g., as described herein. In other embodiments, providing an anellovector comprises obtaining an anellovector or a preparation thereof, e.g., from a third party.

In some embodiments of any of the aforesaid anellovectors, anellovectors, compositions or methods, the genetic element comprises an anellovector genome, e.g., as identified according to the method described in Example 9. In embodiments, the anellovector genome is an anellovector genome capable of self-replication and/or self-amplification. In some embodiments, the anellovector genome is not capable of self-replication and/or self-amplification. In some embodiments, the anellovector genome is capable of replicating and/or being amplified in trans, e.g., in the presence of a helper, e.g., a helper virus.

Additional features of any of the aforesaid anellovectors, anelloVLPs, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. A particle comprising:
a proteinaceous exterior comprising about 40-80 (e.g., about 60), 100-140 (e.g., about 120), or 160-200 (e.g., about 180) copies of an Anellovirus ORF1 molecule,
wherein the particle:
   (i) does not comprise (e.g., does not enclose) a polynucleotide (e.g., as determined using a nuclease protection assay as described herein),
   (ii) does not comprise (e.g., does not enclose) a polynucleotide of greater than 1000, 500, 200, or 100 nucleotides in length, or
   (iii) comprises less than about 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.
2. The particle of embodiment 1, wherein the Anellovirus ORF1 molecule comprises an ORF1 domain and an exogenous surface moiety.
3. The particle of embodiment 1, wherein the Anellovirus ORF1 molecule is bound to an exogenous surface moiety via a noncovalent integration or a covalent bond other than a peptide bond.
4. The particle of embodiment 1, wherein the Anellovirus ORF1 molecule does not comprise a structural arginine-rich domain.
5. The particle of embodiment 1, wherein the particle is a virus-like particle (VLP).
6. A particle comprising:
(a) a proteinaceous exterior comprising about 40-80 (e.g., about 60), 100-140 (e.g., about 120), or 160-200 (e.g., about 180) copies of an Anellovirus ORF1 molecule and an exogenous surface moiety, and (b) a genetic element comprising a heterologous nucleic acid sequence encoding an exogenous effector.
7. A particle comprising:
a proteinaceous exterior comprising an Anellovirus ORF1 molecule, wherein the ORF1 molecule comprises an ORF1 domain and an exogenous surface moiety;
wherein one or more of:
a) the exogenous surface moiety is chosen from a receptor, a ligand, an antibody molecule (e.g., scFv), an antigen (e.g., a viral antigen, a bacterial antigen, a fungal antigen, or a parasite antigen) an adjuvant (e.g., TLR agonist, e.g., bacterial flagellin);
b) wherein the ORF1 molecule comprises a hypervariable region (HVR);
c) wherein the particle comprises a genetic element that encodes a peptide or polypeptide that boosts an immune response (e.g. an adjuvant, a TCR agonist (e.g., a bacterial flagellin));
d) wherein the exogenos surface moiety is between 1-5, 5-10, 10-20, 20-50, 50-100, 100-200, 200-500, or 500-1000 amino acids in length;
e) wherein a polypeptide linker region is situated between the exogenous surface moiety and the ORF1 molecule,
f) wherein the particle comprises 1-2, 2-5, 5-10, 10-20, 20-40, 40-60, 60-80, 80-100, 100-125, 125-150, 150-175, 175-200, 200-225, 225-250, 250-275, or 275-300 copies of the exogenous surface moiety;
g) wherein the proteinaceous exterior comprises (i) a plurality of ORF1 molecules lacking the exogenous surface moiety (e.g., a wild-type ORF1 molecule) and (ii) a plurality of ORF1 molecules that comprise the exogenous surface moiety, wherein optionally the ratio of (i) (ii) is between 10:1-5:1, 5:1-2:1, 2:1-1:2, 1:2-1:5, or 1:5-1:10; and/or
h) wherein the particle further comprises a second exogenous surface moiety.
8. The particle of embodiment 7, wherein the exogenous surface moiety is situated at an insertion point between an N-terminal portion of the ORF1 domain and a C-terminal portion of the ORF1 domain.
9. The particle of embodiment 8, wherein the insertion point is in the HVR.
10. The particle of any of embodiments 7-9, further comprising a genetic element comprising a heterologous nucleic acid sequence encoding an exogenous effector.
11. The particle of any of embodiments 7-10, wherein the particle does not comprise (e.g., does not enclose) a polynucleotide, or does not comprise (e.g., does not enclose) a polynucleotide of greater than 1000, 500, 200, or 100 nucleotides in length, or comprises less than about 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.
12. A particle comprising:
a proteinaceous exterior comprising an Anellovirus ORF1 molecule, and an exogenous surface moiety, wherein the exogenous surface moiety is covalently bound to the ORF1 molecule using a bond other than a peptide bond.
13. The particle of embodiment 12, wherein the exogenous surface moiety is attached to an NHS moiety, and the exogenous surface moiety is bound to the ORF1 molecule via the NHS moiety.
14. The particle of embodiment 12, wherein a non-polypeptide linker is situated between the exogenous surface moiety and the ORF1 molecule.
15. The particle of embodiment 14, wherein the non-polypeptide linker comprises a click linkage.
16. The particle of embodiment 14, wherein the non-polypeptide linker is produced by a click reaction between a DBCO moiety and an azide moiety.
17. The particle of embodiment 16, wherein, prior to the click reaction, the DBCO moiety is attached to the Anellovirus ORF1 molecule via an NHS moiety.
18. The particle of embodiment 16 or 17, wherein, prior to the click reaction, the azide moiety is attached to the exogenous surface moiety via an NHS moiety.
19. The particle of embodiment 17 or 18, wherein the NHS moiety of the DBCO moiety is attached to a lysine residue on the surface of the Anellovirus ORF1 molecule.
20. The particle of any of embodiments 17-19, wherein the NHS moiety of the azide moiety is attached to a lysine residue on the surface of the exogenous surface moiety.
21. The particle of embodiment 16, wherein, prior to the click reaction, the DBCO moiety is attached to the exogenous surface moiety via an NHS moiety.
22. The particle of embodiment 16 or 21, wherein, prior to the click reaction, the azide moiety is attached to the Anellovirus ORF1 molecule via an NHS moiety.
23. The particle of embodiment 21 or 22, wherein the NHS moiety of the DBCO moiety is attached to a lysine residue on the surface of the exogenous surface moiety.
24. The particle of any of embodiments 21-23, wherein the NHS moiety of the azide moiety is attached to a lysine residue on the surface of the Anellovirus ORF1 molecule.
25. A particle comprising:
a proteinaceous exterior comprising an Anellovirus ORF1 molecule, and an exogenous surface moiety, wherein the exogenous surface moiety is non-covalently bound to the ORF1 molecule.
26. The particle of embodiment 25, wherein the ORF1 molecule comprises an exogenous binding domain (e.g., MS2 coat protein or avidin), and the exogenous surface moiety comprises a cognate binding moiety (e.g., MS2 hairpin or biotin) that binds the exogenous binding domain.
27. The particle of any of embodiments 12-26, wherein the exogenous surface moiety comprises a polypeptide.
28. The particle of any of embodiments 12-27, wherein the exogenous surface moiety comprises a small molecule or nucleic acid molecule (e.g., polynucleotide).
29. The particle of any of the preceding embodiments, wherein the ratio of ORF1 molecule to exogenous surface moiety is between about 60:1-30:1, 30:1-20:1, 20:1-10:1, or 10:1-1:1. 30. The particle of any of the preceding embodiments, wherein the antibody molecule is a bispecific antibody molecule.
31. The particle of embodiment 30, wherein the bispecific antibody molecule comprises:
a first antigen-binding domain that binds a first antigen on a first type of host cell, and
a second antigen-binding domain that binds a second antigen on a second type of host cell.
32. The particle of any of the preceding embodiments, which is capable of entering a target cell, e.g., by endocytosis.
33. The particle of embodiment 32, wherein the exogenous surface moiety binds to a cognate moiety in the target cell.
34. The particle of embodiment 32, wherein the particle comprises a genetic element encoding an exogenous effector to be delivered to the interior of the target cell.
35. The particle of any of the preceding embodiments, wherein a genetic element is enclosed within the proteinaceous exterior.
36. The particle of any of the preceding embodiments, which particle does not comprise a polynucleotide, or does not comprise a polynucleotide of greater than 1000, 500, 200, or 100 nucleotides in length.
37. The particle of any of the preceding embodiments, wherein the Anellovirus ORF1 molecule comprises:
(b) a first region comprising an Anellovirus ORF1 structural jelly-roll region, e.g., an amino acid sequence having at least 30% (e.g., at least about 30, 35, 40, 50, 60, 70, 80, 90, 95, 96, 97, 98, 99, or 100%) sequence identity to a Anellovirus ORF1 structural jelly-roll region sequence described herein or a sequence comprising at least 6 beta strands;
(c) a second region comprising an Anellovirus ORF1 structural N22 domain, e.g., an amino acid sequence having at least 30% (e.g., at least about 30, 35, 40, 50, 60, 70, 80, 90, 95, 96, 97, 98, 99, or 100%) sequence identity to an Anellovirus ORF1 structural N22 domain sequence described herein; and
(d) a third region comprising an Anellovirus ORF1 structural C-terminal domain (CTD), e.g., an amino acid sequence having at least 30% (e.g., at least about 30, 35, 40, 50, 60, 70, 80, 90, 95, 96, 97, 98, 99, or 100%) sequence identity to an Anellovirus ORF1 CTD sequence described herein; and
wherein the Anellovirus ORF1 molecule does not comprise an Anellovirus ORF1 structural arginine-rich region, e.g., a sequence of at least about 40 amino acids comprising at least 60%, 70%, or 80% basic residues (e.g., arginine, lysine, or a combination thereof).
38. A preparation comprising the particle of any of the preceding embodiments.
39. The preparation of embodiment 38, wherein the preparation comprises less than 10¹⁰-10¹⁴ (e.g., less than 10¹⁰-10¹¹, 10¹¹-10¹², 10¹²-10¹³, or 10¹³-10¹⁴) viral genome equivalents of nucleic acid molecules (e.g., genetic elements, e.g., of an anellovector as described herein) per kilogram of a subject to be administered the composition (e.g., as determined by qPCR or by measuring optical density).
40. A polypeptide, e.g., an Anellovirus ORF1 molecule, comprising:
(b) a first region comprising an Anellovirus ORF1 structural jelly-roll region, e.g., an amino acid sequence having at least 30% (e.g., at least about 30, 35, 40, 50, 60, 70, 80, 90, 95, 96, 97, 98, 99, or 100%) sequence identity to a Anellovirus ORF1 structural jelly-roll region sequence described herein or a sequence comprising at least 6 beta strands;
(c) a second region comprising an Anellovirus ORF1 structural N22 domain, e.g., an amino acid sequence having at least 30% (e.g., at least about 30, 35, 40, 50, 60, 70, 80, 90, 95, 96, 97, 98, 99, or 100%) sequence identity to an Anellovirus ORF1 structural N22 domain sequence described herein; and
(d) a third region comprising an Anellovirus ORF1 structural C-terminal domain (CTD), e.g., an amino acid sequence having at least 30% (e.g., at least about 30, 35, 40, 50, 60, 70, 80, 90, 95, 96, 97, 98, 99, or 100%) sequence identity to an Anellovirus ORF1 CTD sequence described herein; and
wherein the polypeptide does not comprise an Anellovirus ORF1 structural arginine-rich region, e.g., a sequence of at least about 40 amino acids comprising at least 60%, 70%, or 80% basic residues (e.g., arginine, lysine, or a combination thereof).
41. A nucleic acid molecule encoding a polypeptide of embodiment 40.
42. A particle comprising a proteinaceous exterior comprising an Anellovirus ORF1 molecule, wherein the ORF1 molecule comprises an ORF1 domain and an exogenous surface domain;
wherein the particle is made by contacting a plurality of Anellovirus ORF1 molecules in a cell-free solution under conditions suitable to form a proteinaceous exterior comprising the plurality of Anellovirus ORF1 molecules.
43. The particle of embodiment 42, wherein the particle does not comprise (e.g., does not enclose) a polynucleotide, or does not comprise (e.g., does not enclose) a polynucleotide of greater than 1000, 500, 200, or 100 nucleotides in length.
44. A method of making a particle, the method comprising:
contacting a plurality of Anellovirus ORF1 molecules in a cell-free solution under conditions suitable to form a proteinaceous exterior comprising the plurality of Anellovirus ORF1 molecules;
thereby making a particle.
45. A method of modulating a biological activity in a cell, the method comprising: contacting the cell with a particle of any of the proceeding embodiments;
wherein the cell comprises a moiety on its surface that binds to the exogenous surface moiety of the particle.
46. A method of targeting a particle to a cell, the method comprising:
contacting the cell with a particle of any of the proceeding embodiments;
   wherein the cell comprises a moiety on its surface that binds to the exogenous surface moiety of the particle.
47. A polypeptide comprising (e.g., in an N to C-terminal direction):
   (i) a structural jelly-roll region of an Anellovirus ORF1 molecule;
   (ii) a structural N22 domain of an Anellovirus ORF1 molecule; and
   (iii) a portion of a structural C-terminal domain (CTD) of an Anellovirus ORF1 molecule, which comprises a deletion of about 20-30, 30-40 (e.g., about 37), 40-50 (e.g., about 55), 50-60, 60-70, 70-80, 80-90, 90-100, 100-110, 110-120, 120-130 (e.g., about 129), 130-140 (e.g., about 131), 140-150 (e.g., about 148), or 150-160 (e.g., about 155) amino acids at the C-terminal end of the structural CTD, relative to a corresponding wild-type structural CTD of the Anellovirus ORF1 molecule.
48. A polypeptide comprising (e.g., in an N to C-terminal direction):
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   (vi) a portion of a structural C-terminal domain (CTD) of an Anellovirus ORF1 molecule, which comprises a deletion of about 20-30, 30-40 (e.g., about 37), 40-50 (e.g., about 55), 50-60, 60-70, 70-80, 80-90, 90-100, 100-110, 110-120, 120-130 (e.g., about 129), 130-140 (e.g., about 131), 140-150 (e.g., about 148), or 150-160 (e.g., about 155) amino acids at the C-terminal end of the structural CTD, relative to a corresponding wild-type structural CTD of the Anellovirus ORF1 molecule.
49. A polypeptide comprising (e.g., in an N to C-terminal direction):
   (i) a structural jelly-roll region of an Anellovirus ORF1 molecule;
   (ii) an structural N22 domain of an Anellovirus ORF1 molecule; and
   (iii) a portion of a structural CTD of an Anellovirus ORF1 molecule, wherein the portion consists of the N-terminal-most 1-5, 5-10 (e.g. about 7), 10-20, 30-40, 40-50, 50-60 (e.g. about 52), 60-70 (e.g., about 69), 70-80, 80-90 (e.g., about 88), 90-100 (e.g., about 93), or 100-110 amino acids of a corresponding wild-type structural CTD of an Anellovirus ORF1 molecule, or a sequence having at least 80%, 85%, 90%, 95%, 96%, 97%, 98%, or 99% identity thereto.
50. A polypeptide comprising (e.g., in an N to C-terminal direction):
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   (vi) a portion of a structural CTD of an Anellovirus ORF1 molecule, wherein the portion consists of the N-terminal most 1-5, 5-10 (e.g. about 7), 10-20, 30-40, 40-50, 50-60 (e.g. about 52), 60-70 (e.g., about 69), 70-80, 80-90 (e.g., about 88), 90-100 (e.g., about 93), or 100-110 amino acids of a corresponding wild-type structural CTD of an Anellovirus ORF1 molecule, or a sequence having at least 80%, 85%, 90%, 95%, 96%, 97%, 98%, or 99% identity thereto.
51. A polypeptide comprising (e.g., in an N to C-terminal direction):
   (i) a structural jelly-roll region of an Anellovirus ORF1 molecule comprising the structural jelly-roll sequence of a Ring2, Ring9, Ring10, Ring 18, or Ring19 Anellovirus ORF1 protein (e.g., as described herein), or an amino acid sequence having at least 75%, 80%, 85%, 90%, 95%, 96%, 97%, 98%, or 99% identity thereto; and
   (ii) a full-length structural N22 domain of an Anellovirus ORF1 molecule comprising the structural N22 sequence of the Ring2, Ring9, Ring10, Ring 18, or Ring19 Anellovirus ORF1 protein, or an amino acid sequence having at least 75%, 80%, 85%, 90%, 95%, 96%, 97%, 98%, or 99% identity thereto;
   wherein the polypeptide does not comprise the amino acid sequence of the full-length structural C-terminal domain of the Ring2, Ring9, Ring10, Ring 18, or Ring19 Anellovirus ORF1 protein.
52. A polypeptide comprising (e.g., in an N to C-terminal direction):
   (i) optionally the amino acid sequence of beta strands B—H of the structural jelly-roll region of a Ring2, Ring9, Ring10, Ring 18, or Ring19 Anellovirus ORF1 protein (e.g., as described herein), or an amino acid sequence having at least 75%, 80%, 85%, 90%, 95%, 96%, 97%, 98%, or 99% identity thereto;
   (ii) the amino acid sequence of a full-length P1-1 domain of the Ring2, Ring9, Ring10, Ring 18, or Ring19 Anellovirus ORF1 protein, or an amino acid sequence having at least 75%, 80%, 85%, 90%, 95%, 96%, 97%, 98%, or 99% identity thereto;
   (iii) the amino acid sequence of a full-length P2 domain of the Ring2, Ring9, Ring10, Ring 18, or Ring19 Anellovirus ORF1 protein, or an amino acid sequence having at least 75%, 80%, 85%, 90%, 95%, 96%, 97%, 98%, or 99% identity thereto;
   (iv) the amino acid sequence of a full-length P1-2 domain of the Ring2, Ring9, Ring10, Ring 18, or Ring19 Anellovirus ORF1 protein, or an amino acid sequence having at least 75%, 80%, 85%, 90%, 95%, 96%, 97%, 98%, or 99% identity thereto; and
   (v) optionally the amino acid sequence of beta strand I of the structural jelly-roll region of the Ring2, Ring9, Ring10, Ring 18, or Ring19 Anellovirus ORF1 protein (e.g., as described herein), or an amino acid sequence having at least 75%, 80%, 85%, 90%, 95%, 96%, 97%, 98%, or 99% identity thereto;
   wherein the polypeptide does not comprise the amino acid sequence of the full-length structural C-terminal domain of the Ring2, Ring9, Ring10, Ring 18, or Ring19 Anellovirus ORF1 protein.
53. The polypeptide of embodiment 51 or 52, which comprises a fragment of the structural C-terminal domain.
54. The polypeptide of embodiment 51 or 52, which does not comprise a fragment of the structural C-terminal domain.
55. The polypeptide of any of embodiments 47-54, which further comprises (e.g., at the C-terminal end of the C-terminal P1 subdomain sequence) a structural jelly-roll I region of an Anellovirus ORF1 molecule comprising the structural jelly-roll I sequence as listed in Table A2, or an amino acid sequence having at least 75%, 80%, 85%, 90%, 95%, 96%, 97%, 98%, or 99% identity thereto.
56. The polypeptide of any of embodiments 47-55, wherein the polypeptide does not comprise the 1, 2, 3, 4, 5, 6, 7, 8, 9 or 10 most N-terminal amino acid residues of the structural jelly-roll B—H region of the Anellovirus ORF1 molecule.
57. The polypeptide of any of the preceding embodiments, further comprising a structural arginine-rich domain of an Anellovirus ORF1 molecule, e.g., wherein the structural arginine-rich domain is N-terminal of the structural jelly-roll region.
58. The polypeptide of any of embodiments 47-57, further comprising an amino acid sequence having at least 70% (e.g., at least about 70, 80, 90, 95, 96, 97, 98, 99, or 100%) sequence identity to a structural arginine-rich region sequence described herein, or a sequence of at least about 40 amino acids comprising at least 60%, 70%, or 80% basic residues.
59. The polypeptide of any of embodiments 47-58, wherein the polypeptide further comprises a deletion of a structural arginine-rich region of an Anellovirus ORF1 molecule.
60. The polypeptide of any of embodiments 47-59, wherein the polypeptide comprises a deletion of 35-40, 40-45, 45-50, 50-55, 55-60, 60-65, or 65-70 amino acids (e.g., 37, 40, 43, 44, 47 49, 66, or 67 amino acids) relative to a corresponding wild-type arginine-rich region of an Anellovirus ORF1 molecule.
61. The polypeptide of any of embodiments 47-60, wherein the polypeptide does not comprise a structural arginine-rich region of an Anellovirus ORF1 molecule.
62. The polypeptide of any of the preceding embodiments, wherein the polypeptide does not comprise a sequence of at least 60 contiguous amino acids consisting of at least 40%, 45%, 50%, 55%, 60%, 65%, 70%, or 75% (e.g., up to 80%) basic residues.
63. The polypeptide of any of the preceding embodiments, wherein the polypeptide does not comprise a sequence of at least 60 contiguous amino acids consisting of at least 40%, 45%, 50%, 55%, 60%, 65%, or 75% (e.g., up to 80%) arginine residues.
64. The polypeptide of any of the preceding embodiments, wherein the polypeptide does not comprise a sequence of at least 60 contiguous amino acids consisting of at least 2%, 3%, 4%, 5%, 10%, or 15% (e.g., up to 20%) lysine residues.
65. The polypeptide of any of the preceding embodiments, wherein the polypeptide does not comprise a sequence of at least 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous amino acids consisting of at least 40%, 41%, 42%, 43%, 44%, 45%, 46%, 47%, 48%, 49%, 50%, 51%, 52%, 53%, 54%, 55%, 56%, 57%, 58%, 59%, or 60% arginine residues.
66. The polypeptide of any of the preceding embodiments, further comprising a structural hypervariable region (HVR) of an Anellovirus ORF1 molecule (e.g., situated between the structural jelly-roll region and the structural N22 domain).
67. A polypeptide comprising (e.g., in an N-terminal to C-terminal direction):
   (i) a structural jelly-roll region of an Anellovirus ORF1 molecule; and
   (ii) an structural N22 domain of an Anellovirus ORF1 molecule; and
   wherein the polypeptide lacks a structural C-terminal domain (CTD) of an Anellovirus ORF1 molecule.
68. A polypeptide comprising (e.g., in an N-terminal to C-terminal direction):
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   wherein the polypeptide lacks a structural C-terminal domain (CTD) of an Anellovirus ORF1 molecule.
69. A polypeptide comprising (e.g., in an N-terminal to C-terminal direction):
   (i) a structural jelly-roll region of an Anellovirus ORF1 molecule, or an amino acid sequence having at least 75%, 80%, 85%, 90%, 95%, 96%, 97%, 98%, or 99% identity thereto; and
   (ii) an structural N22 domain of an Anellovirus ORF1 molecule, or an amino acid sequence having at least 75%, 80%, 85%, 90%, 95%, 96%, 97%, 98%, or 99% identity thereto; and
   wherein the polypeptide lacks a structural arginine-rich region of an Anellovirus ORF1 molecule.
70. A polypeptide comprising (e.g., in an N-terminal to C-terminal direction):
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   wherein the polypeptide lacks a structural arginine-rich region of an Anellovirus ORF1 molecule.
71. A polypeptide comprising (e.g., in an N-terminal to C-terminal direction):
   (i) a portion of a full-length structural jelly-roll region of an Anellovirus ORF1 molecule, which does not comprise the 1-10 (e.g., 7) N-terminal-most amino acids of the full-length structural jelly-roll region; and
   (ii) an structural N22 domain of the Anellovirus ORF1 molecule; and
   wherein the polypeptide lacks a structural arginine-rich region of the Anellovirus ORF1 molecule.
72. A polypeptide comprising (e.g., in an N-terminal to C-terminal direction):
   (i) a portion of the full-length beta strands B—H of the structural jelly-roll region of an Anellovirus ORF1 molecule, which does not comprise the 1-10 (e.g., 7) N-terminal-most amino acids of the full-length beta strands B—H of the structural jelly-roll region;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   wherein the polypeptide lacks a structural arginine-rich region of the Anellovirus ORF1 molecule.
73. The polypeptide of embodiment 71 or 72, wherein the Anellovirus ORF1 molecule is a Ring2 ORF1 molecule.
74. The polypeptide of embodiment 71 or 72, wherein the Anellovirus ORF1 molecule is a Ring9 ORF1 molecule.
75. The polypeptide of embodiment 71 or 72, wherein the Anellovirus ORF1 molecule is a Ring10 ORF1 molecule.
76. The polypeptide of embodiment 71 or 72, wherein the Anellovirus ORF1 molecule is a Ring18 ORF1 molecule.
77. The polypeptide of embodiment 71 or 72, wherein the Anellovirus ORF1 molecule is a Ring19 ORF1 molecule.
78. The polypeptide of any of embodiments 69-77, wherein the polypeptide does not comprise the 1, 2, 3, 4, 5, 6, 7, 8, 9 or 10 most N-terminal amino acid residues of the structural jelly-roll region of the Anellovirus ORF1 molecule.
79. A polypeptide comprising:
   (i) a structural jelly-roll region of an Anellovirus ORF1 molecule;
   (ii) an structural N22 domain of an Anellovirus ORF1 molecule; and
   (iii) an amino acid sequence comprising substitutions of at least 50%, 60%, 70%, 80%, or 90% of basic amino acids relative to the structural arginine-rich region of a wild-type Anellovirus ORF1 molecule.
80. A polypeptide comprising:
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   (vi) an amino acid sequence comprising substitutions of at least 50%, 60%, 70%, 80%, or 90% of basic amino acids relative to the structural arginine-rich region of a wild-type Anellovirus ORF1 molecule.
81. The polypeptide of embodiment 79 or 80, wherein (iii) comprises one or more (e.g., 1, 2, 3, 4, 5, 6, 7, 8, 9, or 10) mutations of lysine to histidine relative to the structural arginine-rich region of the wild-type Anellovirus ORF1 molecule.
82. The polypeptide of embodiment 79 or 80, which comprises the amino acid sequencenumbered 1721 in Table B4-2, or an amino acid sequence having at least 75%, 80%, 85%, 90%, 95%, 96%, 97%, 98%, or 99% identity thereto.
83. The polypeptide of embodiment 79, wherein the amino acid sequence of (iii) further comprises one or more substitutions at one or more acidic, hydrophobic, and/or nonpolar amino acids of the structural arginine-rich region of the wild-type Anellovirus ORF1 molecule.
84. The polypeptide of any of embodiments 69-83, which further comprises a structural CTD, e.g., C-terminal of the structural N22 domain.
85. The polypeptide of any of embodiments 69-84, which further comprises a structural HVR, e.g., situated between the structural jelly-roll region and the structural N22 domain.
86. The polypeptide of any of embodiments 69-85, which does not substantially bind DNA.
87. The polypeptide of any of embodiments 69-86, further comprising (e.g., at the N-terminus) an N-terminal portion of a capsid protein from a virus other than an Anellovirus, or an amino acid sequence having at least 75%, 80%, 85%, 90%, 95%, 96%, 97%, 98%, or 99% identity thereto;
   optionally wherein the N-terminal region of the capsid protein from the virus other than an Anellovirus comprises at least the N-terminal 10, 20, 30, 40, 41, 42, 43, 44, 45, 46, 47, 48, 49, 50, 55, or 60 amino acids of the capsid protein.
88. The polypeptide of any of embodiments 69-87, further comprising (e.g., at the N-terminus) a structural arginine-rich motif of a capsid protein from a virus other than an Anellovirus, or an amino acid sequence having at least 75%, 80%, 85%, 90%, 95%, 96%, 97%, 98%, or 99% identity thereto.
89. The polypeptide of embodiment 88, wherein the virus other than an Anellovirus is beak and feather disease virus (BFDV).
90. The polypeptide of any of embodiments 69-89, further comprising (e.g., at the N-terminus) the amino acid sequence MWGTSNCACAKFQIRRRYARPYRRRHIRRYRRRRRHFRRRRFTTNR (SEQ ID NO: 2617), or an amino acid sequence having at least 75%, 80%, 85%, 90%, 95%, 96%, 97%, 98%, or 99% identity thereto.
91. A polypeptide comprising (e.g., in an N-terminal to C-terminal direction):
   (i) a structural arginine-rich region of a first Anellovirus ORF1 molecule, or an amino acid sequence having at least 75%, 80%, 85%, 90%, 95%, 96%, 97%, 98%, or 99% identity thereto;
   (ii) a structural jelly-roll region of a second Anellovirus ORF1 molecule, or an amino acid sequence having at least 75%, 80%, 85%, 90%, 95%, 96%, 97%, 98%, or 99% identity thereto; and
   (iii) an structural N22 domain of the second Anellovirus ORF1 molecule, or an amino acid sequence having at least 75%, 80%, 85%, 90%, 95%, 96%, 97%, 98%, or 99% identity thereto;
   wherein the sequence of (i) comprises at least one amino acid sequence difference relative to the structural arginine-rich region of the second Anellovirus ORF1 molecule.
92. A polypeptide comprising (e.g., in an N-terminal to C-terminal direction):
   (i) a structural arginine-rich region of a first Anellovirus ORF1 molecule, or an amino acid sequence having at least 75%, 80%, 85%, 90%, 95%, 96%, 97%, 98%, or 99% identity thereto;
   (ii) optionally a first portion of a structural jelly-roll region (e.g., comprising beta strands B—H of the structural jelly-roll region) of a second Anellovirus ORF1 molecule;
   (iii) a first portion of a P1 domain of a second Anellovirus ORF1 molecule (e.g., a P1-1 domain as described herein);
   (iv) a P2 domain of a second Anellovirus ORF1 molecule;
   (v) a second portion of a P1 domain of a second Anellovirus ORF1 molecule (e.g., a P1-2 domain as described herein);
   (vi) optionally a second portion of a structural jelly-roll region (e.g., comprising beta strand I of the structural jelly-roll region) of a second Anellovirus ORF1 molecule; and
   wherein the sequence of (i) comprises at least one amino acid sequence difference relative to the structural arginine-rich region of the second Anellovirus ORF1 molecule.
93. The polypeptide of embodiment 91 or 92, wherein (i) comprises the structural arginine-rich region of the first Anellovirus ORF1 molecule, or an amino acid sequence having at least 90% identity thereto.
94. The polypeptide of embodiment 91 or 92, wherein (i) comprises the structural arginine-rich region of the first Anellovirus ORF1 molecule, or an amino acid sequence having at least 95% identity thereto.
95. The polypeptide of embodiment 91 or 92, wherein (i) comprises the structural arginine-rich region of the first Anellovirus ORF1 molecule, or an amino acid sequence having at least 97% identity thereto.
96. The polypeptide of embodiment 91 or 92, wherein (i) has 100% sequence identity to the structural arginine-rich region of the first Anellovirus ORF1 molecule.
97. The polypeptide of any of embodiments 91-96, wherein the first Anellovirus ORF1 molecule is Ring 9 and the second Anellovirus ORF1 molecule is Ring 2.
98. The polypeptide of any of embodiments 91-97, wherein the first Anellovirus ORF1 molecule is Ring 2 and the second Anellovirus ORF1 molecule is Ring 9.
99. A polypeptide comprising (e.g., in an N-terminal to C-terminal direction):
   (i) an N-terminal portion of a first Anellovirus ORF1 molecule, or an amino acid sequence having at least 75%, 80%, 85%, 90%, 95%, 96%, 97%, 98%, or 99% identity thereto, wherein the N-terminal portion of the first Anellovirus ORF1 molecule has a length of between 30-40, 40-50, 50-60, 60-70, 70-80, or 80-90 amino acids; and
   (ii) a C-terminal portion of a second Anellovirus ORF1 molecule, or an amino acid sequence having at least 75%, 80%, 85%, 90%, 95%, 96%, 97%, 98%, or 99% identity thereto, wherein the C-terminal portion of the second Anellovirus ORF1 molecule has a length of between 590-600, 600-610, 610-620, 620-630, 630-640, 640-650, 650-660, 660-670, 670-680, 680-690, 690-700, 700-710, 710-720, or 720-730 amino acids; and
   wherein the sequence of (i) comprises at least one amino acid sequence difference relative to the structural arginine-rich region of the second Anellovirus ORF1 molecule.
100. The polypeptide of embodiment 99, wherein the N-terminal portion of the first Anellovirus ORF1 molecule comprises a structural arginine-rich domain.
101. The polypeptide of embodiment 99 or 100, wherein the N-terminal portion of the first Anellovirus ORF1 molecule further comprises an N-terminal portion of a structural jelly-roll domain (e.g., having a length of between 1-10, 10-20, 20-30, 30-40, or 40-50 amino acids).
102. The polypeptide of any of embodiments 99-101, wherein the N-terminal portion of the first Anellovirus ORF1 molecule further comprises one or more beta strands of a structural jelly-roll domain (e.g., beta strands 1 and/or 2 of the structural jelly-roll domain).
103. The polypeptide of any of embodiments 99-102, wherein the C-terminal portion of the second Anellovirus ORF1 molecule comprises a C-terminal portion of a structural jelly-roll domain (e.g., having a length of between 100-110, 110-120, 120-130, 130-140, 140-150, 150-160, 160-170, 170-180, 180-190, or 190-200 amino acids).
104. A polypeptide comprising (e.g., in an N-terminal to C-terminal direction):
   (i) a first portion of a structural jelly-roll region of a first Anellovirus ORF1 molecule, or an amino acid sequence having at least 75%, 80%, 85%, 90%, 95%, 96%, 97%, 98%, or 99% identity thereto; and
   (ii) a second portion of a structural jelly-roll region of a second Anellovirus ORF1 molecule, or an amino acid sequence having at least 75%, 80%, 85%, 90%, 95%, 96%, 97%, 98%, or 99% identity thereto; and
   wherein the sequence of (i) and (ii) together comprises at least one amino acid sequence difference relative to each of the sequences of the structural jelly-roll region of the first Anellovirus ORF1 molecule and the second Anellovirus ORF1 molecule.
105. A polypeptide comprising (e.g., in an N-terminal to C-terminal direction):
   (i) a structural jelly-roll region of an Anellovirus ORF1 molecule; and
   (ii) an structural N22 domain of an Anellovirus ORF1 molecule; and
   wherein the polypeptide lacks a structural arginine-rich region of an Anellovirus ORF1 molecule; and
   wherein the polypeptide lacks a structural C-terminal domain of an Anellovirus ORF1 molecule.
106. A polypeptide comprising (e.g., in an N-terminal to C-terminal direction):
   (i) optionally a first portion of a structural jelly-roll region (e.g., comprising beta strands B—H of the structural jelly-roll region) of an Anellovirus ORF1 molecule;
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   wherein the polypeptide lacks a structural arginine-rich region of an Anellovirus ORF1 molecule; and
   wherein the polypeptide lacks a structural C-terminal domain of an Anellovirus ORF1 molecule.
107. The polypeptide of any of the preceding embodiments, wherein the polypeptide does not comprise amino acids 2-45 of the amino acid sequence of a Ring2 ORF1 protein as described herein, or an amino acid sequence having at least 75%, 80%, 85%, 90%, 95%, 96%, 97%, 98%, or 99% identity thereto.
108. The polypeptide of any of the preceding embodiments, wherein the polypeptide does not comprise amino acids 518-666 of the amino acid sequence of a Ring2 ORF1 protein as described herein, or an amino acid sequence having at least 75%, 80%, 85%, 90%, 95%, 96%, 97%, 98%, or 99% identity thereto.
109. The polypeptide of any of the preceding embodiments, wherein the polypeptide does not comprise amino acids 538-666 of the amino acid sequence of a Ring2 ORF1 protein as described herein, or an amino acid sequence having at least 75%, 80%, 85%, 90%, 95%, 96%, 97%, 98%, or 99% identity thereto.
110. The polypeptide of any of the preceding embodiments, wherein the polypeptide does not comprise amino acids 545-666 of the amino acid sequence of a Ring2 ORF1 protein as described herein, or an amino acid sequence having at least 75%, 80%, 85%, 90%, 95%, 96%, 97%, 98%, or 99% identity thereto.
111. The polypeptide of any of the preceding embodiments, wherein the polypeptide does not comprise amino acids 594-666 of the amino acid sequence of a Ring2 ORF1 protein as described herein, or an amino acid sequence having at least 75%, 80%, 85%, 90%, 95%, 96%, 97%, 98%, or 99% identity thereto.
112. The polypeptide of any of the preceding embodiments, wherein the polypeptide does not comprise amino acids 611-666 of the amino acid sequence of a Ring2 ORF1 protein as described herein, or an amino acid sequence having at least 75%, 80%, 85%, 90%, 95%, 96%, 97%, 98%, or 99% identity thereto.
113. The polypeptide of any of the preceding embodiments, wherein the polypeptide does not comprise amino acids 630-666 of the amino acid sequence of a Ring2 ORF1 protein as described herein, or an amino acid sequence having at least 75%, 80%, 85%, 90%, 95%, 96%, 97%, 98%, or 99% identity thereto.
114. The polypeptide of any of the preceding embodiments, wherein the polypeptide does not comprise amino acids 2-45 of the amino acid sequence of a Ring10 ORF1 protein as described herein, or an amino acid sequence having at least 75%, 80%, 85%, 90%, 95%, 96%, 97%, 98%, or 99% identity thereto.
115. The polypeptide of any of the preceding embodiments, wherein the polypeptide does not comprise amino acids 2-51 of the amino acid sequence of a Ring10 ORF1 protein as described herein, or an amino acid sequence having at least 75%, 80%, 85%, 90%, 95%, 96%, 97%, 98%, or 99% identity thereto.
116. The polypeptide of any of the preceding embodiments, wherein the polypeptide does not comprise amino acids 542-672 of the amino acid sequence of a Ring10 ORF1 protein as described herein, or an amino acid sequence having at least 75%, 80%, 85%, 90%, 95%, 96%, 97%, 98%, or 99% identity thereto.
117. The polypeptide of any of the preceding embodiments, wherein the polypeptide does not comprise amino acids 552-672 of the amino acid sequence of a Ring10 ORF1 protein as described herein, or an amino acid sequence having at least 75%, 80%, 85%, 90%, 95%, 96%, 97%, 98%, or 99% identity thereto.
118. The polypeptide of any of the preceding embodiments, wherein the polypeptide does not comprise amino acids 557-672 of the amino acid sequence of a Ring10 ORF1 protein as described herein, or an amino acid sequence having at least 75%, 80%, 85%, 90%, 95%, 96%, 97%, 98%, or 99% identity thereto.
119. The polypeptide of any of the preceding embodiments, wherein the polypeptide does not comprise amino acids 560-672 of the amino acid sequence of a Ring10 ORF1 protein as described herein, or an amino acid sequence having at least 75%, 80%, 85%, 90%, 95%, 96%, 97%, 98%, or 99% identity thereto.
120. The polypeptide of any of the preceding embodiments, wherein the polypeptide does not comprise amino acids 566-672 of the amino acid sequence of a Ring10 ORF1 protein as described herein, or an amino acid sequence having at least 75%, 80%, 85%, 90%, 95%, 96%, 97%, 98%, or 99% identity thereto.
121. The polypeptide of any of the preceding embodiments, wherein the polypeptide does not comprise amino acids 569-672 of the amino acid sequence of a Ring10 ORF1 protein as described herein, or an amino acid sequence having at least 75%, 80%, 85%, 90%, 95%, 96%, 97%, 98%, or 99% identity thereto.
122. The polypeptide of any of the preceding embodiments, wherein the polypeptide does not comprise amino acids 610-672 of the amino acid sequence of a Ring10 ORF1 protein as described herein, or an amino acid sequence having at least 75%, 80%, 85%, 90%, 95%, 96%, 97%, 98%, or 99% identity thereto.
123. The polypeptide of any of the preceding embodiments, wherein the polypeptide does not comprise amino acids 2-69 of the amino acid sequence of a Ring18 ORF1 protein as described herein, or an amino acid sequence having at least 75%, 80%, 85%, 90%, 95%, 96%, 97%, 98%, or 99% identity thereto.
124. The polypeptide of any of the preceding embodiments, wherein the polypeptide does not comprise amino acids 582-736 of the amino acid sequence of a Ring18 ORF1 protein as described herein, or an amino acid sequence having at least 75%, 80%, 85%, 90%, 95%, 96%, 97%, 98%, or 99% identity thereto.
125. The polypeptide of any of the preceding embodiments, wherein the polypeptide does not comprise amino acids 651-736 of the amino acid sequence of a Ring18 ORF1 protein as described herein, or an amino acid sequence having at least 75%, 80%, 85%, 90%, 95%, 96%, 97%, 98%, or 99% identity thereto.
126. The polypeptide of any of the preceding embodiments, wherein the polypeptide does not comprise amino acids 2-49 of the amino acid sequence of a Ring19 ORF1 protein as described herein, or an amino acid sequence having at least 75%, 80%, 85%, 90%, 95%, 96%, 97%, 98%, or 99% identity thereto.
127. The polypeptide of any of the preceding embodiments, wherein the polypeptide does not comprise amino acids 527-655 of the amino acid sequence of a Ring19 ORF1 protein as described herein, or an amino acid sequence having at least 75%, 80%, 85%, 90%, 95%, 96%, 97%, 98%, or 99% identity thereto.
128. The polypeptide of any of the preceding embodiments, wherein the polypeptide does not comprise amino acids 537-655 of the amino acid sequence of a Ring19 ORF1 protein as described herein, or an amino acid sequence having at least 75%, 80%, 85%, 90%, 95%, 96%, 97%, 98%, or 99% identity thereto.
129. The polypeptide of any of the preceding embodiments, wherein the polypeptide does not comprise amino acids 583-655 of the amino acid sequence of a Ring19 ORF1 protein as described herein, or an amino acid sequence having at least 75%, 80%, 85%, 90%, 95%, 96%, 97%, 98%, or 99% identity thereto.
130. The polypeptide of any of the preceding embodiments, wherein the polypeptide does not comprise amino acids 600-655 of the amino acid sequence of a Ring19 ORF1 protein as described herein, or an amino acid sequence having at least 75%, 80%, 85%, 90%, 95%, 96%, 97%, 98%, or 99% identity thereto.
131. A polypeptide comprising (e.g., in an N-terminal to C-terminal direction):
   (i) a mutant ORF1 structural jelly-roll region; and
   (ii) an structural N22 domain of an Anellovirus ORF1 molecule;
   wherein the mutant ORF1 structural jelly-roll region comprises one or more mutations (e.g., at least 2, 3, 4, 5, 6, 7, 8, 9, or 10 mutations) in a beta strand relative to the amino acid sequence of a wild-type Anellovirus ORF1 structural jelly-roll region.
132. A polypeptide comprising (e.g., in an N-terminal to C-terminal direction):
   (i) a mutant ORF1 structural jelly-roll region; and
   (ii) a first portion of a P1 domain of an Anellovirus ORF1 molecule (e.g., a P1-1 domain as described herein);
   (iii) a P2 domain of an Anellovirus ORF1 molecule;
   (iv) a second portion of a P1 domain of an Anellovirus ORF1 molecule (e.g., a P1-2 domain as described herein);
   (v) optionally a second portion of a structural jelly-roll region (e.g., comprising beta strand I of the structural jelly-roll region) of an Anellovirus ORF1 molecule; and
   wherein the mutant ORF1 structural jelly-roll region comprises one or more mutations (e.g., at least 2, 3, 4, 5, 6, 7, 8, 9, or 10 mutations) in a beta strand relative to the amino acid sequence of a wild-type Anellovirus ORF1 structural jelly-roll region.
133. The polypeptide of embodiment 131 or 132, wherein the one or more mutations in the beta strand comprise one or more mutations (e.g., at least 2, 3, 4, 5, 6, 7, 8, 9, or 10 mutations) of a basic residue in the amino acid sequence of the wild-type Anellovirus ORF1 structural jelly-roll region, e.g., to a residue other than a basic residue (e.g., a nonpolar residue or an acidic residue).
134. The polypeptide of any of embodiments 131-133, wherein the beta strand is selected from beta strand B, D, G, or I of the wild-type Anellovirus ORF1 structural jelly-roll region.
135. The polypeptide of any of embodiments 131-134, wherein the one or more mutations reduce the binding of the polypeptide to a nucleic acid molecule relative to an otherwise similar polypeptide comprising the wild-type Anellovirus ORF1 structural jelly-roll region.
136. A polypeptide comprising (e.g., in an N to C-terminal direction):
   (i) optionally a structural arginine-rich region of an Anellovirus ORF1 (e.g., a full-length arginine-rich region or a portion of a structural arginine-rich region),
   (ii) a structural jelly-roll region of an Anellovirus ORF1 molecule,
   (iii) a P1 domain of an Anellovirus ORF1 molecule,
   (iv) a P2 domain of an Anellovirus ORF1 molecule, and
   (v) optionally a structural C-terminal domain (CTD) of an Anellovirus ORF1 molecule (e.g., a full-length structural CTD or a portion of a structural CTD);
   wherein:
      (a) the P1 domain is from a different Anellovirus than one or more (e.g., 1, 2, 3, or 4) of the structural arginine-rich region, structural jelly-roll region, P2 domain, and/or the structural CTD;
      (b) the P2 domain is from a different Anellovirus than one or more (e.g., 1, 2, 3, or 4) of the structural arginine-rich region, structural jelly-roll region, P1 domain, and/or the structural CTD; or
      (c) the P1 domain and the P2 domain are from a different Anellovirus than one or more (e.g., 1, 2, or 3) of the structural arginine-rich region, structural jelly-roll region, and/or the structural CTD, optionally wherein the P1 domain and the P2 domain are from the same Anellovirus.
137. A polypeptide comprising (e.g., in an N to C-terminal direction):
   (i) optionally a structural arginine-rich region of an Anellovirus ORF1 molecule (e.g., a full-length arginine-rich region or a portion of a structural arginine-rich region),
   (ii) a structural jelly-roll region of an Anellovirus ORF1 molecule,
   (iii) a P1 domain of an Anellovirus ORF1 molecule,
   (iv) a P2 domain of a viral capsid protein, wherein the viral capsid protein is not an Anellovirus ORF1 molecule, and
   (v) optionally a structural C-terminal domain (CTD) of an Anellovirus ORF1 molecule (e.g., a full-length structural CTD or a portion of a structural CTD).
138. A polypeptide comprising (e.g., in an N to C-terminal direction):
   (i) optionally a structural arginine-rich region of an Anellovirus ORF1 (e.g., a full-length arginine-rich region or a portion of a structural arginine-rich region),
   (ii) a structural jelly-roll region of an Anellovirus ORF1 molecule,
   (iii) a P1 domain of a viral capsid protein, wherein the viral capsid protein is not an Anellovirus ORF1 molecule,
   (iv) a P2 domain of an Anellovirus ORF1 molecule, and
   (v) optionally a structural C-terminal domain (CTD) of an Anellovirus ORF1 molecule (e.g., a full-length structural CTD or a portion of a structural CTD).
139. The polypeptide of embodiment 137 or 138, wherein the viral capsid protein is a hepatitis virus capsid protein (e.g., a hepatitis E virus capsid protein).
140. A polypeptide comprising (e.g., in an N to C-terminal direction):
   (i) a portion (e.g., an N-terminal portion) of a viral capsid protein, wherein the viral capsid protein is not from an Anellovirus; and
   (ii) a P2 domain of an Anellovirus ORF1 molecule (e.g., a wild-type Anellovirus ORF1 protein), or an amino acid sequence having at least 75%, 80%, 85%, 90%, 95%, 96%, 97%, 98%, or 99% amino acid sequence identity thereto.
141. The polypeptide of embodiment 140, wherein the viral capsid protein is a hepatitis virus capsid protein (e.g., a hepatitis E virus (HEV) capsid protein), or a protein having at least 75%, 80%, 85%, 90%, 95%, 96%, 97%, 98%, or 99% amino acid sequence identity thereto.
142. The polypeptide of embodiment 140 or 141, wherein the portion of the viral capsid protein comprises the amino acid sequence MAVAPAPDTAPVPDVDSRGAILRRQYNLSTSPLTSTIATGTNLVLYAAPLSSLLPLQDGTNTHIM ATEASNYAQYRVVRATIRYRPLVPSAVGGYAISISFWPQTTTTPTSVDMNSITSTDVRILVQPGIAS ELVIPSERLHYRNQGWRSVETSGVAEEEATSGLVMLCIHGSPVNSYTNTPYTGALGLLDFALELE FRNLTPGNTNTRVSRYSSSARHKLRRGPDGTAELTTTAATRFMKDLHFTGTNDVGEVGRGIALT LFNLADTLLGGLPTELISSAGGQLFYSRPVVSANGEPTVKLYTSVENAQQDKGIAIPHDIDLGESR VVIQDYDNQHEQDRPTPSPAPSRP (SEQ ID NO: 2500), or an amino acid sequence having at least 75%, 80%, 85%, 90%, 95%, 96%, 97%, 98%, or 99% amino acid sequence identity thereto.
143. The polypeptide of any of embodiments 140-142, wherein the P2 domain of the amino acid sequence comprises the amino acid sequence LNTTYIQNRDWGDRNKTYYCQTLGTQRYFLYGTHSTAQNINDIKLQELIPLTNTQDYVQGFDWT EKDKHNITTYKEFLTKGAGNPFHAEWITAQNPVIHTANSPTQIEQIYTASTTTFQNKKLTDLPTPG YIFITPTV (SEQ ID NO: 2501), or an amino acid sequence having at least 75%, 80%, 85%, 90%, 95%, 96%, 97%, 98%, or 99% amino acid sequence identity thereto.
144. The polypeptide of any of embodiments 140-143, further comprising one or more additional portions of the viral capsid protein (e.g., at the C-terminal end of the polypeptide).
145. The polypeptide of embodiment 144, wherein the additional portion of the viral capsid protein comprises the amino acid sequence HHHHH (SEQ ID NO: 2502) (e.g., at the C-terminal end of the polypeptide).
146. The polypeptide of any of embodiments 136-145, wherein the amino acid sequence of the P1 domain is non-contiguous (e.g., wherein the amino acid sequence of the P2 domain is situated between a first portion and a second portion of the amino acid sequence of the P1 domain).
147. A polypeptide comprising (e.g., in an N to C-terminal direction) one or more of:
   (i) a structural arginine-rich region of an Anellovirus ORF1 molecule,
   (ii) a structural jelly-roll region of the Anellovirus ORF1 molecule,
   (iii) a structural N22 domain of the Anellovirus ORF1 molecule, and/or
   (iv) a structural C-terminal domain of the Anellovirus ORF1 molecule;
   wherein the polypeptide further comprises (e.g., between the structural jelly-roll region and the structural N22 domain e.g., in a structural hypervariable region (HVR) as described herein) the amino acid sequence EQI, e.g., the amino acid sequence SPTQIEQIYT (SEQ ID NO: 2503); and
   wherein the Anellovirus ORF1 molecule of (i)-(iv) comprises at least one difference relative to a Ring 10 ORF1 protein.
148. A polypeptide comprising (e.g., in an N to C-terminal direction) one or more of:
   (i) a structural arginine-rich region of an Anellovirus ORF1 molecule,
   (ii) a jelly-roll B—H strands subdomain of the Anellovirus ORF1 molecule,
   (iii) a P1-1 subdomain of the Anellovirus ORF1 molecule,
   (iv) a P1-2 subdomain of the Anellovirus ORF1 molecule,
   (v) a jelly-roll I strand subdomain of the Anellovirus ORF1 molecule, and/or
   (vi) a structural C-terminal domain of the Anellovirus ORF1 molecule;
   wherein the polypeptide further comprises (e.g., between the P1-1 subdomain and the P1-2 subdomain domain) the amino acid sequence EQI, e.g., the amino acid sequence SPTQIEQIYT (SEQ ID NO: 2503) (e.g., in a P2 domain as described herein); and
   wherein the Anellovirus ORF1 molecule of (i)-(vi) comprises at least one difference relative to a Ring 10 ORF1 protein.
149. A polypeptide comprising a structural hypervariable region (HVR) having at least 75%, 80%, 85%, 90%, 95%, 96%, 97%, 98%, or 99% sequence identity to the structural HVR sequence of a Ring 2 ORF1 protein;
   wherein the polypeptide comprises one or more (e.g., 1, 2, or all 3) amino acid substitutions relative to the sequence of the Ring 2 ORF1 protein, the substitutions selected from K357E, N358Q, and E359I.
150. A polypeptide comprising a P2 domain having at least 75%, 80%, 85%, 90%, 95%, 96%, 97%, 98%, or 99% sequence identity to the P2 domain sequence of a Ring 2 ORF1 protein;
   wherein the polypeptide comprises one or more (e.g., 1, 2, or all 3) amino acid substitutions relative to the sequence of the Ring 2 ORF1 protein, the substitutions selected from K357E, N358Q, and E359I.
151. The polypeptide of embodiment 149 or 150, wherein the polypeptide comprises all three of the substitutions selected from K357E, N358Q, and E359I.
152. A method of enriching a polypeptide of any of embodiments X1-X3b, the method comprising:
   (a) providing the polypeptide (e.g., wherein the polypeptide is comprised in an anelloVLP or a particle);
   (b) contacting the polypeptide with a binding moiety (e.g., an antibody molecule) that binds an epitope comprising the amino acid sequence EQI, and
   (c) enriching material bound by the binding moiety relative to material not bound by the binding moiety,
   thereby enriching the polypeptide.
153. An anellovector comprising:
   (i) a proteinaceous exterior comprising a polypeptide of any of the preceding embodiments; and
   (ii) a genetic element enclosed by the proteinaceous exterior, wherein the genetic element comprises a sequence encoding an exogenous effector.
154. An anellovector comprising:
   (i) a proteinaceous exterior comprising a plurality of Anellovirus ORF1 molecules, wherein the Anellovirus ORF1 molecule lacks part or all of a structural C-terminal domain (CTD); and
   (ii) a genetic element enclosed by the proteinaceous exterior, wherein the genetic element comprises a sequence encoding an exogenous effector.
155. An anellovector comprising:
   (i) a proteinaceous exterior comprising a plurality of Anellovirus ORF1 molecules, wherein the Anellovirus ORF1 molecule lacks part or all of a structural arginine-rich region; and
   (ii) a genetic element enclosed by the proteinaceous exterior, wherein the genetic element comprises a sequence encoding an exogenous effector.
156. A composition comprising a plurality of the anellovectors of any of embodiments 153-155.
157. A particle comprising:
   a proteinaceous exterior comprising a plurality of Anellovirus ORF1 molecules, wherein the Anellovirus ORF1 molecule lacks part or all of a structural C-terminal domain (CTD);
   wherein the particle:
      (i) does not comprise (e.g., does not enclose) a polynucleotide (e.g., as determined using a nuclease protection assay as described herein),
      (ii) does not comprise (e.g., does not enclose) a polynucleotide of greater than 1000, 500, 200, or 100 nucleotides in length, or
      (iii) comprises less than about 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.
158. A particle comprising:
   a proteinaceous exterior comprising a plurality of Anellovirus ORF1 molecules, wherein the Anellovirus ORF1 molecule lacks part or all of a structural arginine-rich region;
   wherein the particle:
      (i) does not comprise (e.g., does not enclose) a polynucleotide (e.g., as determined using a nuclease protection assay as described herein),
      (ii) does not comprise (e.g., does not enclose) a polynucleotide of greater than 1000, 500, 200, or 100 nucleotides in length, or
      (iii) comprises less than about 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.
159. A particle comprising:
   a proteinaceous exterior comprising a plurality of Anellovirus ORF1 molecules, wherein the Anellovirus ORF1 molecule comprises a polypeptide of any of the preceding embodiments;
   wherein the particle:
      (i) does not comprise (e.g., does not enclose) a polynucleotide (e.g., as determined using a nuclease protection assay as described herein),
      (ii) does not comprise (e.g., does not enclose) a polynucleotide of greater than 1000, 500, 200, or 100 nucleotides in length, or
      (iii) comprises less than about 1, 2, 3, 4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.
160. The particle of any of embodiments 157-159, wherein the proteinaceous exterior comprises about 40-80 (e.g., about 60), 100-140 (e.g., about 120), or 160-200 (e.g., about 180) copies of the Anellovirus ORF1 molecules.
161. The particle of any of embodiments 157-160, wherein the particle further comprises an exogenous effector or a nucleic acid sequence encoding an exogenous effector.
162. A composition comprising a plurality of the particles of any of embodiments 157-161.
163. A nucleic acid molecule encoding a polypeptide of any of the preceding embodiments.
164. A method of delivering an exogenous effector to a cell, the method comprising contacting a cell with an anellovector or particle of any of the preceding embodiments, or a particle (e.g., an anellovector) comprising a polypeptide of any of the preceding embodiments, thereby delivering the exogenous effector to the cell.
165. A method of delivering an exogenous effector to a subject, the method comprising administering to the subject an anellovector or particle of any of the preceding embodiments, or a particle (e.g., an anellovector) comprising a polypeptide of any of the preceding embodiments, thereby delivering the exogenous effector to the subject.
166. A method of treating or preventing a disease or disorder in a subject, the method comprising administering to a subject in need thereof an anellovector or particle of any of the preceding embodiments, or a particle (e.g., an anellovector) comprising a polypeptide of any of the preceding embodiments, thereby treating or preventing the disease or disorder in the subject.
167. A method of making an anellovector, the method comprising:
   (a) providing a host cell comprising:
      (i) a plurality of Anellovirus ORF1 molecules, wherein the Anellovirus ORF1 molecule comprises a polypeptide of any of the preceding embodiments, and
      (ii) a genetic element, wherein the genetic element comprises a sequence encoding an exogenous effector; and
   (b) maintaining the host cell under conditions that allow for production of a proteinaceous exterior that comprises the plurality of Anellovirus ORF1 molecules, wherein the proteinaceous exterior encloses the genetic element,
   thereby producing one or more of the anellovectors from the host cell.
168. A method of making an anellovector, the method comprising:
   (a) providing a host cell comprising:
      (i) a plurality of Anellovirus ORF1 molecules, wherein the Anellovirus ORF1 molecule lacks part or all of a structural C-terminal domain (CTD), and
      (ii) a genetic element, wherein the genetic element comprises a sequence encoding an exogenous effector; and
   (b) maintaining the host cell under conditions that allow for production of a proteinaceous exterior that comprises the plurality of Anellovirus ORF1 molecules, wherein the proteinaceous exterior encloses the genetic element;
   thereby producing one or more of the anellovectors from the host cell.
169. A method of making an anellovector, the method comprising:
   (a) providing a host cell comprising:
      (i) a plurality of Anellovirus ORF1 molecules, wherein the Anellovirus ORF1 molecule lacks part or all of a structural arginine-rich region, and
      (ii) a genetic element, wherein the genetic element comprises a sequence encoding an exogenous effector; and
   (b) maintaining the host cell under conditions that allow for production of a proteinaceous exterior that comprises the plurality of Anellovirus ORF1 molecules, wherein the proteinaceous exterior encloses the genetic element;
   thereby producing one or more of the anellovectors from the host cell.
170. The method of any of embodiments 167-169, wherein providing the host cell comprising the plurality of Anellovirus ORF1 molecules comprises maintaining the host cell under conditions that allow for expression of the Anellovirus ORF1 molecules.
171. A method of making an anellovector, the method comprising:
   (a) providing a mixture comprising a plurality of Anellovirus ORF1 molecules, wherein the Anellovirus ORF1 molecule comprises a polypeptide of any of the preceding embodiments; optionally subjecting the mixture to denaturing conditions (e.g., providing a denaturant as part of the mixture or contacting the mixture with a denaturant), wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to non-denaturing conditions (e.g., reducing the concentration of the denaturant to a level) suitable for in vitro assembly of the Anellovirus ORF1 molecules (e.g., by dialysis); and
   (c) incubating the Anellovirus ORF1 molecules with a plurality of genetic elements, under conditions suitable for assembly of the Anellovirus ORF1 molecules into one or more anellovectors each enclosing one or more of the genetic elements.
172. A method of making an anellovector, the method comprising:
   (a) providing a mixture comprising a plurality of Anellovirus ORF1 molecules, wherein the Anellovirus ORF1 molecule lacks part or all of a structural C-terminal domain (CTD); optionally subjecting the mixture to denaturing conditions (e.g., providing a denaturant as part of the mixture or contacting the mixture with a denaturant), wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to non-denaturing conditions (e.g., reducing the concentration of the denaturant to a level) suitable for in vitro assembly of the Anellovirus ORF1 molecules (e.g., by dialysis); and
   (c) incubating the Anellovirus ORF1 molecules with a plurality of genetic elements, under conditions suitable for assembly of the Anellovirus ORF1 molecules into one or more anellovectors each enclosing one or more of the genetic elements.
173. A method of making an anellovector, the method comprising:
   (a) providing a mixture comprising a plurality of Anellovirus ORF1 molecules, wherein the Anellovirus ORF1 molecule lacks part or all of a structural arginine-rich region; optionally subjecting the mixture to denaturing conditions (e.g., providing a denaturant as part of the mixture or contacting the mixture with a denaturant), wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to non-denaturing conditions (e.g., reducing the concentration of the denaturant to a level) suitable for in vitro assembly of the Anellovirus ORF1 molecules (e.g., by dialysis); and
   (c) incubating the Anellovirus ORF1 molecules with a plurality of genetic elements, under conditions suitable for assembly of the Anellovirus ORF1 molecules into one or more anellovectors each enclosing one or more of the genetic elements.
174. A method of making an anelloVLP, the method comprising:
   (a) providing a mixture comprising a plurality of Anellovirus ORF1 molecules, wherein the Anellovirus ORF1 molecule comprises a polypeptide of any of the preceding embodiments, and wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to conditions suitable for in vitro assembly of the Anellovirus ORF1 molecules; and
   (c) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.
175. A method of making an anelloVLP, the method comprising:
   (a) providing a mixture comprising a plurality of Anellovirus ORF1 molecules, wherein the Anellovirus ORF1 molecule lacks part or all of a structural C-terminal domain (CTD), and wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to conditions suitable for in vitro assembly of the Anellovirus ORF1 molecules; and
   (c) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.
176. A method of making an anelloVLP, the method comprising:
   (a) providing a mixture comprising a plurality of Anellovirus ORF1 molecules, wherein the Anellovirus ORF1 molecule lacks part or all of a structural arginine-rich region, and wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
   (b) subjecting the mixture to conditions suitable for in vitro assembly of the Anellovirus ORF1 molecules; and
   (c) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.
177. The polypeptide, particle, nucleic acid molecule, or method of any of the preceding embodiments, wherein the polypeptide or Anellovirus ORF1 molecule comprises an exogenous surface moiety.
178. The polypeptide, particle, nucleic acid molecule, or method of embodiment 177, wherein the exogenous surface moiety is fused to the N-terminus of the Anellovirus ORF1 molecule.
179. The polypeptide, particle, nucleic acid molecule, or method of embodiment 177, wherein the exogenous surface moiety is fused to the C-terminus of the Anellovirus ORF1 molecule.
180. The polypeptide, particle, nucleic acid molecule, or method of embodiment 177, wherein the exogenous surface moiety is inserted within the amino acid sequence of the Anellovirus ORF1 molecule.
181. The polypeptide, particle, nucleic acid molecule, or method of any of embodiments 178-180, wherein the Anellovirus ORF1 molecule comprises a portion of a structural C-terminal domain (CTD) of an Anellovirus ORF1 molecule, which comprises a deletion of about 20-30, 30-40 (e.g., about 37), 40-50 (e.g., about 55), 50-60, 60-70, 70-80, 80-90, 90-100, 100-110, 110-120, 120-130 (e.g., about 129), 130-140 (e.g., about 131), 140-150 (e.g., about 148), or 150-160 (e.g., about 155) amino acids at the C-terminal end of the structural CTD, relative to a corresponding wild-type structural CTD of the Anellovirus ORF1 molecule.
182. The polypeptide, particle, nucleic acid molecule, or method of any of embodiments 178-180, wherein the Anellovirus ORF1 molecule comprises a portion of a structural CTD of an Anellovirus ORF1 molecule, wherein the portion consists of the N-terminal most 1-5, 5-10 (e.g. about 7), 10-20, 30-40, 40-50, 50-60 (e.g. about 52), 60-70 (e.g., about 69), 70-80, 80-90 (e.g., about 88), 90-100 (e.g., about 93), or 100-110 amino acids of a corresponding wild-type structural CTD of an Anellovirus ORF1 molecule, or a sequence having at least 80%, 85%, 90%, 95%, 96%, 97%, 98%, or 99% identity thereto.
183. The polypeptide, particle, nucleic acid molecule, or method of any of the preceding embodiments, wherein the polypeptide or Anellovirus ORF1 molecule comprises a detectable marker or effector (e.g., exogenous effector).
184. The polypeptide, particle, nucleic acid molecule, or method of embodiment 183, wherein the detectable marker or effector is fused to the N-terminus of the Anellovirus ORF1 molecule.
185. The polypeptide, particle, nucleic acid molecule, or method of embodiment 183, wherein the detectable marker or effector is fused to the C-terminus of the Anellovirus ORF1 molecule.
186. The polypeptide, particle, nucleic acid molecule, or method of embodiment 183, wherein the detectable marker or effector is inserted within the amino acid sequence of the Anellovirus ORF1 molecule.
187. The polypeptide, particle, nucleic acid molecule, or method of any one of embodiments 183-186, wherein the detectable marker comprises an epitope tag, e.g., a His tag or a FLAG tag.
188. The polypeptide, particle, nucleic acid molecule, or method of any one of embodiments 183-187, wherein the detectable marker comprises a fluorescent protein (e.g., GFP).
189. The polypeptide, particle, nucleic acid molecule, or method of any one of embodiments 183-188, wherein the polypeptide or Anellovirus ORF1 molecule comprises a protease recognition sequence (e.g., a 3C protease recognition sequence) between the detectable marker or effector and the remainder of the polypeptide or Anellovirus ORF1 molecule.
190. The polypeptide, particle, nucleic acid molecule, or method of any one of embodiments 183-189, wherein the effector is a therapeutic effector (e.g., a therapeutic polypeptide or therapeutic nucleic acid molecule).
191. The polypeptide, particle, nucleic acid molecule, or method of any of the preceding embodiments, wherein the polypeptide or Anellovirus ORF1 molecule comprises at its N-terminus a methionine residue corresponding to the N-terminal methionine residue of an Anellovirus structural arginine-rich region.
192. The polypeptide, particle, nucleic acid molecule, or method of any of the preceding embodiments, wherein the Anellovirus ORF1 molecule is a Ring 2 ORF1 molecule (e.g., as described herein), or an ORF1 molecule having at least 75%, 80%, 90%, 95%, 96%, 97%, 98%, 99%, or 100% sequence identity thereto.
193. The polypeptide, particle, nucleic acid molecule, or method of any of the preceding embodiments, wherein the Anellovirus ORF1 molecule is an ORF1 molecule of SEQ ID NO: 58, or an ORF1 molecule having at least 75%, 80%, 90%, 95%, 96%, 97%, 98%, 99%, or 100% sequence identity thereto.
194. The polypeptide, particle, nucleic acid molecule, or method of any of the preceding embodiments, wherein the Anellovirus ORF1 molecule is a Ring 9 ORF1 molecule (e.g., as described herein), or an ORF1 molecule having at least 75%, 80%, 90%, 95%, 96%, 97%, 98%, 99%, or 100% sequence identity thereto.
195. The polypeptide, particle, nucleic acid molecule, or method of any of the preceding embodiments, wherein the Anellovirus ORF1 molecule is an ORF1 molecule of SEQ ID NO: 1005, or an ORF1 molecule having at least 75%, 80%, 90%, 95%, 96%, 97%, 98%, 99%, or 100% sequence identity thereto.
196. The polypeptide, particle, nucleic acid molecule, or method of any of the preceding embodiments, wherein the Anellovirus ORF1 molecule is a Ring 10 ORF1 molecule (e.g., as described herein), or an ORF1 molecule having at least 75%, 80%, 90%, 95%, 96%, 97%, 98%, 99%, or 100% sequence identity thereto.
197. The polypeptide, particle, nucleic acid molecule, or method of any of the preceding embodiments, wherein the Anellovirus ORF1 molecule is an ORF1 molecule of SEQ ID NO: 1012, or an ORF1 molecule having at least 75%, 80%, 90%, 95%, 96%, 97%, 98%, 99%, or 100% sequence identity thereto.
198. The polypeptide, particle, nucleic acid molecule, or method of any of the preceding embodiments, wherein the Anellovirus ORF1 molecule is a Ring 18 ORF1 molecule (e.g., as described herein), or an ORF1 molecule having at least 75%, 80%, 90%, 95%, 96%, 97%, 98%, 99%, or 100% sequence identity thereto.
199. The polypeptide, particle, nucleic acid molecule, or method of any of the preceding embodiments, wherein the Anellovirus ORF1 molecule is an ORF1 molecule of SEQ ID NO: 1100, or an ORF1 molecule having at least 75%, 80%, 90%, 95%, 96%, 97%, 98%, 99%, or 100% sequence identity thereto.
200. The polypeptide, particle, nucleic acid molecule, or method of any of the preceding embodiments, wherein the Anellovirus ORF1 molecule is a Ring 19 ORF1 molecule (e.g., as described herein), or an ORF1 molecule having at least 75%, 80%, 90%, 95%, 96%, 97%, 98%, 99%, or 100% sequence identity thereto.
201. The polypeptide, particle, nucleic acid molecule, or method of any of the preceding embodiments, wherein the Anellovirus ORF1 molecule is an ORF1 molecule of Table B1-11a, or an ORF1 molecule having at least 75%, 80%, 90%, 95%, 96%, 97%, 98%, 99%, or 100% sequence identity thereto.
202. The polypeptide, particle, nucleic acid molecule, or method of any of the preceding embodiments, wherein the polypeptide or Anellovirus ORF1 molecule comprises an amino acid sequence as shown in any of Tables B1-1 to B4-5, or an amino acid sequence having at least 75%, 80%, 90%, 95%, 96%, 97%, 98%, 99%, or 100% sequence identity thereto.
203. The polypeptide, particle, nucleic acid molecule, or method of any of the preceding embodiments, wherein the polypeptide or Anellovirus ORF1 molecule comprises an amino acid sequence as shown in any of Tables B1-1 to B4-5, or an amino acid sequence having at least 90% sequence identity thereto.
204. The polypeptide, particle, nucleic acid molecule, or method of any of the preceding embodiments, wherein the polypeptide or Anellovirus ORF1 molecule comprises an amino acid sequence as shown in any of Tables B1-1 to B4-5, or an amino acid sequence having at least 95% sequence identity thereto.
205. The polypeptide, particle, nucleic acid molecule, or method of any of the preceding embodiments, wherein the polypeptide or Anellovirus ORF1 molecule comprises an amino acid sequence as shown in any of Tables B1-1 to B4-5, or an amino acid sequence having at least 99% sequence identity thereto.
206. The polypeptide, particle, nucleic acid molecule, or method of any of the preceding embodiments, wherein the polypeptide or Anellovirus ORF1 molecule comprises the structural arginine-rich region sequence of Ring 2, Ring 9, Ring 10, Ring 18, or Ring 19, or an amino acid sequence having at least 75%, 80%, 90%, 95%, 96%, 97%, 98%, or 99% sequence identity thereto.
207. The polypeptide, particle, nucleic acid molecule, or method of any of the preceding embodiments, wherein the polypeptide or Anellovirus ORF1 molecule comprises the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
208. The polypeptide, particle, nucleic acid molecule, or method of any of the preceding embodiments, wherein the polypeptide or Anellovirus ORF1 molecule comprises the structural hypervariable domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
209. The polypeptide, particle, nucleic acid molecule, or method of any of the preceding embodiments, wherein the polypeptide or Anellovirus ORF1 molecule comprises the structural N22 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
210. The polypeptide, particle, nucleic acid molecule, or method of any of the preceding embodiments, wherein the polypeptide or Anellovirus ORF1 molecule comprises the structural C-terminal domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
211. The polypeptide, particle, nucleic acid molecule, or method of any of the preceding embodiments, wherein the polypeptide or Anellovirus ORF1 molecule does not comprise the structural arginine-rich region sequence of Ring 2, Ring 9, Ring 10, Ring 18, or Ring 19, or an amino acid sequence having at least 75%, 80%, 90%, 95%, 96%, 97%, 98%, or 99% sequence identity thereto.
212. The polypeptide, particle, nucleic acid molecule, or method of any of the preceding embodiments, wherein the polypeptide or Anellovirus ORF1 molecule does not comprise the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
213. The polypeptide, particle, nucleic acid molecule, or method of any of the preceding embodiments, wherein the polypeptide or Anellovirus ORF1 molecule does not comprise the structural hypervariable domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
214. The polypeptide, particle, nucleic acid molecule, or method of any of the preceding embodiments, wherein the polypeptide or Anellovirus ORF1 molecule does not comprise the structural N22 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
215. The polypeptide, particle, nucleic acid molecule, or method of any of the preceding embodiments, wherein the polypeptide or Anellovirus ORF1 molecule does not comprise the structural C-terminal domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
216. The polypeptide, particle, nucleic acid molecule, or method of any of the preceding embodiments, wherein the polypeptide or Anellovirus ORF1 molecule comprises the structural arginine-rich region sequence of Ring 2, Ring 9, Ring 10, Ring 18, or Ring 19, or an amino acid sequence having at least 75%, 80%, 90%, 95%, 96%, 97%, 98%, or 99% sequence identity thereto.
217. The polypeptide, particle, nucleic acid molecule, or method of any of the preceding embodiments, wherein the polypeptide or Anellovirus ORF1 molecule comprises beta strands B—H of the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
218. The polypeptide, particle, nucleic acid molecule, or method of any of the preceding embodiments, wherein the polypeptide or Anellovirus ORF1 molecule comprises the P1-1 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
219. The polypeptide, particle, nucleic acid molecule, or method of any of the preceding embodiments, wherein the polypeptide or Anellovirus ORF1 molecule comprises the P2 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
220. The polypeptide, particle, nucleic acid molecule, or method of any of the preceding embodiments, wherein the polypeptide or Anellovirus ORF1 molecule comprises the P1-2 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
221. The polypeptide, particle, nucleic acid molecule, or method of any of the preceding embodiments, wherein the polypeptide or Anellovirus ORF1 molecule comprises beta strand I of the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
222. The polypeptide, particle, nucleic acid molecule, or method of any of the preceding embodiments, wherein the polypeptide or Anellovirus ORF1 molecule comprises the structural C-terminal domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
223. The polypeptide, particle, nucleic acid molecule, or method of any of the preceding embodiments, wherein the polypeptide or Anellovirus ORF1 molecule does not comprise the structural arginine-rich region sequence of Ring 2, Ring 9, Ring 10, Ring 18, or Ring 19, or an amino acid sequence having at least 75%, 80%, 90%, 95%, 96%, 97%, 98%, or 99% sequence identity thereto.
224. The polypeptide, particle, nucleic acid molecule, or method of any of the preceding embodiments, wherein the polypeptide or Anellovirus ORF1 molecule does not comprise beta strands B—H of the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
225. The polypeptide, particle, nucleic acid molecule, or method of any of the preceding embodiments, wherein the polypeptide or Anellovirus ORF1 molecule does not comprise the P1-1 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
226. The polypeptide, particle, nucleic acid molecule, or method of any of the preceding embodiments, wherein the polypeptide or Anellovirus ORF1 molecule does not comprise the P2 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
227. The polypeptide, particle, nucleic acid molecule, or method of any of the preceding embodiments, wherein the polypeptide or Anellovirus ORF1 molecule does not comprise the P1-2 domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
228. The polypeptide, particle, nucleic acid molecule, or method of any of the preceding embodiments, wherein the polypeptide or Anellovirus ORF1 molecule does not comprise beta strand I of the structural jelly-roll domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
229. The polypeptide, particle, nucleic acid molecule, or method of any of the preceding embodiments, wherein the polypeptide or Anellovirus ORF1 molecule does not comprise the structural C-terminal domain sequence of Ring 2, Ring 9, Ring 10, Ring 18, or Ring 19, or an amino acid sequence having at least 75%, 80%, 90%, 95%, 96%, 97%, 98%, or 99% sequence identity thereto.
230. The polypeptide, particle, nucleic acid molecule, or method of any of the preceding embodiments, wherein the polypeptide or Anellovirus ORF1 molecule comprises one or more (e.g., 1, 2, or all 3) amino acid substitutions relative to the sequence of the Ring 2 ORF1 protein, the substitutions selected from K357E, N358Q, and E359I.
231. The polypeptide, particle, nucleic acid molecule, or method of any of the preceding embodiments, wherein the polypeptide or Anellovirus ORF1 molecule comprises the amino acid sequence SPTQIEQIYT (SEQ ID NO: 2503) (e.g., in a structural HVR domain as described herein).
232. The polypeptide, particle, nucleic acid molecule, or method of any of the preceding embodiments, wherein the polypeptide or Anellovirus ORF1 molecule comprises the amino acid sequence SPTQIEQIYT (SEQ ID NO: 2503) (e.g., in a P2 domain as described herein).
233. An anellovector comprising:
(i) a proteinaceous exterior comprising an Anellovirus ORF1 protein as listed in Table A32, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.
234. An anellovector comprising:
(i) a proteinaceous exterior comprising an Anellovirus ORF1 protein as listed in Table A32, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of a structural arginine-rich region, structural jelly-roll domain, structural HVR, structural N22, structural CTD, P1 domain, or P2 domain, e.g., as described herein) or genomic region (e.g., one or more of a TATA box, cap site, transcriptional start site, 5′ UTR, open reading frame (ORF), poly(A) signal, or GC-rich region, e.g., as described herein).
235. An anellovector comprising:
(i) a proteinaceous exterior comprising a polypeptide encoded by an Anellovirus ORF1 nucleic acid sequence as listed in Table N24, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.
236. An anellovector comprising:
(i) a proteinaceous exterior comprising a polypeptide encoded by an Anellovirus ORF1 nucleic acid sequence as listed in Table N24, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of a structural arginine-rich region, structural jelly-roll domain, structural HVR, structural N22, structural CTD, P1 domain, or P2 domain, e.g., as described herein) or genomic region (e.g., one or more of a TATA box, cap site, transcriptional start site, 5′ UTR, open reading frame (ORF), poly(A) signal, or GC-rich region, e.g., as described herein).
237. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in Table N24, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an exogenous effector.
238. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises: (a) a 5′ UTR conserved domain as listed in Table N24, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an effector (e.g., an exogenous effector or an endogenous effector);
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of a structural arginine-rich region, structural jelly-roll domain, structural HVR, structural N22, structural CTD, P1 domain, or P2 domain, e.g., as described herein) or genomic region (e.g., one or more of a TATA box, cap site, transcriptional start site, 5′ UTR, open reading frame (ORF), poly(A) signal, or GC-rich region, e.g., as described herein).
239. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an exogenous effector, and wherein the genetic element has at least about 70%, 75%, 80%, 85%, 90%, 95%, 96%, 97%, 98%, 99%, or 100% sequence identity to an Anellovirus genome sequence as listed in Table N24.
240. An anellovector comprising:
(i) a proteinaceous exterior (e.g., comprising an Anellovirus ORF1 molecule, e.g., as described herein, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises a promoter element operably linked to a nucleic acid sequence (e.g., a DNA sequence) encoding an effector (e.g., an exogenous effector or an endogenous effector), and wherein the genetic element has at least about 70%, 75%, 80%, 85%, 90%, 95%, 96%, 97%, 98%, 99%, or 100% sequence identity to an Anellovirus genome sequence as listed in Table N24;
wherein the proteinaceous exterior and/or the genetic element comprises at least one difference (e.g., a mutation, chemical modification, or epigenetic alteration) relative to a wild-type Anellovirus ORF1 protein and/or wild-type Anellovirus genome, respectively (e.g., as described herein), e.g., an insertion, substitution, chemical or enzymatic modification, and/or deletion, e.g., a deletion of a domain (e.g., one or more of a structural arginine-rich region, structural jelly-roll domain, structural HVR, structural N22, or structural CTD, e.g., as described herein) or genomic region (e.g., one or more of a TATA box, cap site, transcriptional start site, 5′ UTR, open reading frame (ORF), poly(A) signal, or GC-rich region, e.g., as described herein).
241. An isolated ORF1 molecule comprising the amino acid sequence of an ORF1 as listed in Table A32, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of a structural arginine-rich region, structural jelly-roll domain, structural HVR, structural N22, structural CTD, P1 domain, or P2 domain, e.g., as described herein).
242. An isolated ORF1 molecule comprising the amino acid sequence of the structural jelly-roll domain of an ORF1 as listed in Table A32, or an amino acid sequence having at least about 70%, 75%, 80%, 85%, 90%, 95%, 96%, 97%, 98%, 99%, or 100% sequence identity thereto;
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of a structural arginine-rich region, jelly-roll domain, structural HVR, structural N22, structural CTD, P1 domain, or P2 domain, e.g., as described herein).
243. An isolated ORF2 molecule comprising the amino acid sequence of an ORF2 as listed in Table A32, or an amino acid sequence having at least about 70%, 75%, 80%, 85%, 90%, 95%, 96%, 97%, 98%, 99%, or 100% sequence identity thereto;
wherein the ORF2 molecule comprises at least one difference (e.g., a mutation, chemical modification, or epigenetic alteration) relative to a wild-type ORF2 protein (e.g., as described herein), e.g., an insertion, substitution, chemical or enzymatic modification, and/or deletion, e.g., a deletion of a domain.
244. An isolated nucleic acid molecule (e.g., a genetic element construct or a genetic element) comprising the nucleic acid sequence of a 5′ UTR conserved domain as listed in Table N24, or a nucleic acid sequence having at least 70%, 75%, 80%, 85%, 90%, 95%, 96%, 97%, 98%, or 99% sequence identity thereto.
245. An isolated nucleic acid molecule (e.g., a genetic element construct or a construct for providing an ORF1 molecule in trans, e.g., as described herein) comprising the nucleic acid sequence of an ORF1 gene as listed in Table N24, or a nucleic acid sequence having at least 70%, 75%, 80%, 85%, 90%, 95%, 96%, 97%, 98%, or 99% sequence identity thereto.
246. An isolated nucleic acid molecule (e.g., a genetic element construct or a construct for providing an ORF2 molecule in trans, e.g., as described herein) comprising the nucleic acid sequence of an ORF2 gene as listed in Table N24, or a nucleic acid sequence having at least 70%, 75%, 80%, 85%, 90%, 95%, 96%, 97%, 98%, or 99% sequence identity thereto.
247. An isolated nucleic acid molecule (e.g., a genetic element construct, a genetic element, or a construct for providing an ORF1 or ORF2 molecule in trans, e.g., as described herein) comprising an Anellovirus genome sequence as listed in Table N24, or a nucleic acid sequence having at least 50%, 60%, 70%, 75%, 80%, 85%, 90%, 95%, 96%, 97%, 98%, or 99% sequence identity thereto.
248. A genetic element comprising:
(a) a 5′ UTR conserved domain as listed in Table N24, or a nucleic acid sequence having at least 70%, 75%, 80%, 85%, 90%, 95%, 96%, 97%, 98%, 99%, or 100% sequence identity thereto, and (b) a promoter element operably linked to a nucleic acid sequence (e.g., a DNA sequence) encoding an exogenous effector.
249. A method of manufacturing an anellovector composition, the method comprising:
   (a) providing a cell, e.g., a host cell as described herein;
   (b) introducing a nucleic acid molecule encoding an ORF1 polypeptide as listed in Table A32 (or an amino acid sequence having at least about 70%, 75%, 80%, 85%, 90%, 95%, 96%, 97%, 98%, or 99% sequence identity thereto) into the cell;
   (c) introducing a genetic element construct into the cell (e.g., before, after, or simultaneously with (b)),
   (d) incubating the cell under conditions that allow the cell to produce anellovector; and
   (e) formulating the anellovectors, e.g., as a pharmaceutical composition suitable for administration to a subject,
thereby making the anellovector composition.
250. A method of manufacturing an anellovector composition, the method comprising:
   (a) providing a cell, e.g., a host cell as described herein;
   (b) introducing a nucleic acid molecule encoding an ORF1 polypeptide into the cell;
   (c) introducing a genetic element construct into the cell as listed in Table N24 (or a nucleic acid sequence having at least about 70%, 75%, 80%, 85%, 90%, 95%, 96%, 97%, 98%, or 99% sequence identity thereto) (e.g., before, after, or simultaneously with (b)),
   (d) incubating the cell under conditions that allow the cell to produce anellovector; and
   (e) formulating the anellovectors, e.g., as a pharmaceutical composition suitable for administration to a subject,
thereby making the anellovector composition.
251. A method of making an anellovector, e.g., a synthetic anellovector, comprising:
(a) providing a host cell comprising:
   (i) a nucleic acid molecule, e.g., a first nucleic acid molecule, comprising the nucleic acid sequence of a Anellovirus genome as listed in Table N24 (or a nucleic acid sequence having at least 70%, 75%, 80%, 85%, 90%, 95%, 96%, 97%, 98%, or 99% sequence identity thereto), and
   (ii) a nucleic acid molecule, e.g., a second nucleic acid molecule, encoding one or more of an amino acid sequence chosen from ORF1, ORF2, ORF2/2, ORF2/3, ORF1/1, or ORF1/2, e.g., as listed in Table A1, or an amino acid sequence having at least 70% 75%, 80%, 85%, 90%, 95%, 96%, 97%, 98%, or 99% sequence identity thereto; and
(b) culturing the host cell under conditions suitable to make the anellovector.
252. A method of delivering an effector to an eye of a subject, the method comprising administering to the eye of the subject an anellovector of any of embodiments 233-240.
253. A method of modulating a biological function in an eye of a subject, the method comprising administering the anellovector of any of embodiments 233-240 to the subject.
254. A method of treating an eye disease or disorder in a subject in need thereof, the method comprising administering to the subject an anellovector of any of embodiments 233-240.
255. An ORF1 molecule comprising an exogenous surface moiety, wherein the exogenous surface moiety is fused to, replaces, and/or is situated within an insertion point of an ORF1 domain (e.g., within an HVR or P2).
256. An ORF1 molecule comprising an exogenous surface moiety, wherein the exogenous surface moiety is fused to, replaces, and/or is situated at an insertion point between residues corresponding to positions 284-285 of Ring 10 ORF1, e.g., in an ORF1 domain (e.g., within the HVR or P2 domain).
257. An ORF1 molecule comprising an exogenous surface moiety, wherein the exogenous surface moiety is fused to, replaces, and/or is situated at an insertion point between residues corresponding to positions 328-329 of Ring 10 ORF1, e.g., in an ORF1 domain (e.g., within the HVR or P2 domain).
258. An ORF1 molecule comprising an exogenous surface moiety, wherein the exogenous surface moiety is fused to, replaces, and/or is situated at an insertion point between residues corresponding to positions 256-383 of Ring 10 ORF1, e.g., in an ORF1 domain (e.g., within the HVR or P2 domain).
259. An ORF1 molecule comprising an exogenous surface moiety, wherein the exogenous surface moiety is fused to, replaces, and/or is situated at an insertion point between residues corresponding to positions 251-383 of Ring 10 ORF1, e.g., in an ORF1 domain (e.g., within the HVR or P2 domain).
260. An ORF1 molecule comprising an exogenous surface moiety, wherein the exogenous surface moiety is fused to, replaces, and/or is situated at an insertion point between residues corresponding to positions 251-384 of Ring 10 ORF1, e.g., in an ORF1 domain (e.g., within the HVR or P2 domain).
261. An ORF1 molecule comprising an exogenous surface moiety, wherein the exogenous surface moiety is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR or P2 domain).
262. The ORF1 molecule of any of embodiments 255-261, wherein the exogenous surface moiety forms a pentamer when the ORF1 molecule is complexed with four other ORF1 molecules (e.g., four other copies of the ORF1 molecule).
263. The ORF1 molecule of any of embodiments 255-262, wherein the exogenous surface moiety forms a trimer when the ORF1 molecule is complexed with four other ORF1 molecules (e.g., four other copies of the ORF1 molecule).
264. The ORF1 molecule of any of embodiments 255-263, wherein the exogenous surface moiety forms a dimer when the ORF1 molecule is complexed with four other ORF1 molecules (e.g., four other copies of the ORF1 molecule).
265. A protein complex comprising five ORF1 molecules, wherein each of the ORF1 molecules comprises:
(i) an ORF1 domain, and
(ii) an exogenous surface moiety;
   wherein the exogenous surface moieties of the five ORF1 molecules forms a pentamer.
266. The protein complex of embodiment 265, wherein each of the exogenous surface moieties is fused to, replaces, and/or is situated within an insertion point of an ORF1 domain (e.g., within an HVR or P2) of the corresponding ORF1 molecule.
267. The protein complex of embodiment 265, wherein each of the exogenous surface moieties is fused to, replaces, and/or is situated within an insertion point between residues corresponding to positions 284-285 in an ORF1 domain (e.g., within the HVR or P2 domain) of Ring 10 ORF1.
268. The protein complex of embodiment 265, wherein each of the exogenous surface moieties is fused to, replaces, and/or is situated within an insertion point between residues corresponding to positions 328-329 in an ORF1 domain (e.g., within the HVR or P2 domain) of Ring 10 ORF1.
269. The protein complex of embodiment 265, wherein each of the exogenous surface moieties is fused to, replaces, and/or is situated within an insertion point between residues corresponding to positions 256-383 in an ORF1 domain (e.g., within the HVR or P2 domain) of Ring 10 ORF1.
270. The protein complex of embodiment 265, wherein each of the exogenous surface moieties is fused to, replaces, and/or is situated within an insertion point between residues corresponding to positions 251-383 in an ORF1 domain (e.g., within the HVR or P2 domain) of Ring 10 ORF1.
271. The protein complex of embodiment 265, wherein each of the exogenous surface moieties is fused to, replaces, and/or is situated within an insertion point between residues corresponding to positions 251-384 in an ORF1 domain (e.g., within the HVR or P2 domain) of Ring 10 ORF1.
272. The protein complex of embodiment 265, wherein each of the exogenous surface moieties is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR or P2 domain).
273. The protein complex of any of embodiments 265-272, wherein the exogenous surface moieties of the ORF1 molecules have the same amino acid sequences.
274. The protein complex of any of embodiments 265-273, wherein at least two (e.g., at least 2, 3, 4, or 5) of the exogenous surface moieties of the ORF1 molecules have different amino acid sequences.
275. A protein complex comprising three ORF1 molecules, wherein each of the ORF1 molecules comprises:
(i) an ORF1 domain, and
(ii) an exogenous surface moiety;
   wherein the exogenous surface moieties of the three ORF1 molecules forms a trimer.
276. The protein complex of embodiment 275, wherein each of the exogenous surface moieties is fused to, replaces, and/or is situated within an insertion point of an ORF1 domain (e.g., within an HVR or P2) of the corresponding ORF1 molecule.
277. The protein complex of embodiment 275, wherein each of the exogenous surface moieties is fused to, replaces, and/or is situated within an insertion point between positions 284-285 in an ORF1 domain (e.g., within the HVR or P2 domain) of the corresponding ORF1 molecule.
278. The protein complex of embodiment 275, wherein each of the exogenous surface moieties is fused to, replaces, and/or is situated within an insertion point between positions 328-329 in an ORF1 domain (e.g., within the HVR or P2 domain) of the corresponding ORF1 molecule.
279. The protein complex of embodiment 275, wherein each of the exogenous surface moieties is fused to, replaces, and/or is situated within an insertion point between positions 256-383 in an ORF1 domain (e.g., within the HVR or P2 domain) of the corresponding ORF1 molecule.
280. The protein complex of embodiment 275, wherein each of the exogenous surface moieties is fused to, replaces, and/or is situated within an insertion point between positions 251-383 in an ORF1 domain (e.g., within the HVR or P2 domain) of the corresponding ORF1 molecule.
281. The protein complex of embodiment 275, wherein each of the exogenous surface moieties is fused to, replaces, and/or is situated within an insertion point between positions 251-384 in an ORF1 domain (e.g., within the HVR or P2 domain) of the corresponding ORF1 molecule.
282. The protein complex of embodiment 275, wherein each of the exogenous surface moieties is attached to (e.g., conjugated to) the amino acid residue (e.g., a cysteine residue) at position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of the corresponding ORF1 molecule, e.g., in an ORF1 domain (e.g., within the HVR or P2 domain).
283. The protein complex of any of embodiments 275-282, wherein the exogenous surface moieties of the ORF1 molecules have the same amino acid sequences.
284. The protein complex of any of embodiments 275-283, wherein at least two (e.g., at least 2 or 3) of the exogenous surface moieties of the ORF1 molecules have different amino acid sequences.
285. A protein complex comprising two ORF1 molecules, wherein each of the ORF1 molecules comprises:
(i) an ORF1 domain, and
(ii) an exogenous surface moiety;
   wherein the exogenous surface moieties of the two ORF1 molecules forms a dimer.
286. The protein complex of embodiment 285, wherein each of the exogenous surface moieties is fused to, replaces, and/or is situated within an insertion point of an ORF1 domain (e.g., within an HVR or P2) of the corresponding ORF1 molecule.
287. The protein complex of embodiment 285, wherein each of the exogenous surface moieties is fused to, replaces, and/or is situated within an insertion point between residues corresponding to positions 284-285 in an ORF1 domain (e.g., within the HVR or P2 domain) of Ring 10 ORF1.
288. The protein complex of embodiment 285, wherein each of the exogenous surface moieties is fused to, replaces, and/or is situated within an insertion point between residues corresponding to positions 328-329 in an ORF1 domain (e.g., within the HVR or P2 domain) of Ring 10 ORF1.
289. The protein complex of embodiment 285, wherein each of the exogenous surface moieties is fused to, replaces, and/or is situated within an insertion point between residues corresponding to positions 256-383 in an ORF1 domain (e.g., within the HVR or P2 domain) of Ring 10 ORF1.
290. The protein complex of embodiment 285, wherein each of the exogenous surface moieties is fused to, replaces, and/or is situated within an insertion point between residues corresponding to positions 251-383 in an ORF1 domain (e.g., within the HVR or P2 domain) of Ring 10 ORF1.
291. The protein complex of embodiment 285, wherein each of the exogenous surface moieties is fused to, replaces, and/or is situated within an insertion point between residues corresponding to positions 251-384 in an ORF1 domain (e.g., within the HVR or P2 domain) of Ring 10 ORF1.
292. The protein complex of embodiment 285, wherein each of the exogenous surface moieties is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR or P2 domain).
293. The protein complex of any of embodiments 285-292, wherein the exogenous surface moieties of the two ORF1 molecules have the same amino acid sequences.
294. The protein complex of any of embodiments 285-293, wherein the exogenous surface moieties of the two ORF1 molecules have different amino acid sequences.
295. The polypeptide, particle, nucleic acid molecule, method, or protein complex of any of the preceding embodiments, wherein the polypeptide or ORF1 molecule comprises one or more substitutions of a cysteine residues (e.g., one or more cysteine to alanine substitutions or one or more cysteine to serine substitutions).
296. The polypeptide, particle, nucleic acid molecule, method, or protein complex of any of the preceding embodiments, wherein the polypeptide or ORF1 molecule comprises a cysteine to serine mutation at one or more (e.g., 1, 2, 3, 4, 5, 6, 7, or 8) residues corresponding to position 63, 70, 137, 269, 403, 460, 503, and/or 515 of a Ring 10 ORF1 protein (e.g., as described herein).
297. The polypeptide, particle, nucleic acid molecule, method, or protein complex of any of the preceding embodiments, wherein the polypeptide or ORF1 molecule comprises a cysteine to alanine mutation at one or more (e.g., 1, 2, 3, 4, 5, 6, or 7) residues corresponding to position 63, 137, 269, 403, 460, 503, and/or 515 of a Ring 10 ORF1 protein (e.g., as described herein).
298. The polypeptide, particle, nucleic acid molecule, method, or protein complex of any of the preceding embodiments, wherein the polypeptide or ORF1 molecule comprises a cysteine to serine mutation at the residue corresponding to position 70 of a Ring 10 ORF1 protein (e.g., as described herein).
299. The polypeptide, particle, nucleic acid molecule, method, or protein complex of any of the preceding embodiments, wherein the polypeptide or ORF1 molecule comprises a substitution of an amino acid residue (e.g., a threonine, serine, asparagine, alanine, glutamine, or lysine residue) to cysteine.
300. The polypeptide, particle, nucleic acid molecule, method, or protein complex of embodiment 299, wherein the polypeptide or ORF1 molecule comprises a threonine to cysteine substitution, e.g., at the position corresponding to position 365 of a Ring 10 ORF1 protein (e.g., as described herein).
301. The polypeptide, particle, nucleic acid molecule, method, or protein complex of embodiment 299, wherein the polypeptide or ORF1 molecule comprises a serine to cysteine substitution, e.g., at the position corresponding to position 284 of a Ring 10 ORF1 protein (e.g., as described herein).
302. The polypeptide, particle, nucleic acid molecule, method, or protein complex of embodiment 299, wherein the polypeptide or ORF1 molecule comprises an asparagine to cysteine substitution, e.g., at the position corresponding to position 290 of a Ring 10 ORF1 protein (e.g., as described herein).
303. The polypeptide, particle, nucleic acid molecule, method, or protein complex of embodiment 299, wherein the polypeptide or ORF1 molecule comprises a lysine to cysteine substitution, e.g., at the position corresponding to position 317 of a Ring 10 ORF1 protein (e.g., as described herein).
304. The polypeptide, particle, nucleic acid molecule, method, or protein complex of embodiment 299, wherein the polypeptide or ORF1 molecule comprises a lysine to cysteine substitution, e.g., at the position corresponding to position 324 of a Ring 10 ORF1 protein (e.g., as described herein).
305. The polypeptide, particle, nucleic acid molecule, method, or protein complex of embodiment 299, wherein the polypeptide or ORF1 molecule comprises an alanine to cysteine substitution, e.g., at the position corresponding to position 362 of a Ring 10 ORF1 protein (e.g., as described herein).
306. The polypeptide, particle, nucleic acid molecule, method, or protein complex of embodiment 299, wherein the polypeptide or ORF1 molecule comprises a serine to cysteine substitution, e.g., at the position corresponding to position 363 of a Ring 10 ORF1 protein (e.g., as described herein).
307. The polypeptide, particle, nucleic acid molecule, method, or protein complex of embodiment 299, wherein the polypeptide or ORF1 molecule comprises an asparagine to cysteine substitution, e.g., at the position corresponding to position 369 of a Ring 10 ORF1 protein (e.g., as described herein).
308. The polypeptide, particle, nucleic acid molecule, method, or protein complex of embodiment 299, wherein the polypeptide or ORF1 molecule comprises a lysine to cysteine substitution, e.g., at the position corresponding to position 371 of a Ring 10 ORF1 protein (e.g., as described herein).
309. The polypeptide, particle, nucleic acid molecule, method, or protein complex of embodiment 299, wherein the polypeptide or ORF1 molecule comprises a glutamine to cysteine substitution, e.g., at the position corresponding to position 287 of a Ring 10 ORF1 protein (e.g., as described herein).
310. The polypeptide, particle, nucleic acid molecule, method, or protein complex of embodiment 299, wherein the polypeptide or ORF1 molecule comprises one or more substitutions to cysteine at one or more positions corresponding to Y254, R263, N264, K265, L272, G273, T274, R276, H283, T285, N288, D291, Q308, D311, W312, T313, E314, D316, H318, N319, T321, T328, K329, T341, Q343, T354, Q358, T361, T364, Q368, D374, P376, P378, Y380, and/or 1381 of a Ring 10 ORF1 protein (e.g., as described herein).
311. A particle comprising:
(a) a proteinaceous exterior comprising an ORF1 molecule; and
(b) a genetic element comprising a heterologous nucleic acid sequence encoding an exogenous effector;
wherein the genetic element is enclosed within the proteinaceous exterior; and
wherein the particle has one or more of the following characteristics:
   (i) the genetic element (e.g., a DNA genetic element) does not comprise an Anellovirus 5′ UTR or an origin of replication;
   (ii) the sequence encoding the exogenous effector takes up at least 90%, 95%, 96%, 97%, 98%, 99% or 100% of the genetic element (e.g., a DNA genetic element);
   (iii) the heterologous nucleic acid sequence takes up at least 90%, 95%, 96%, 97%, 98%, 99% or 100% of the genetic element (e.g., a DNA genetic element);
   (iv) the particle does not comprise a detectable amount of (e.g., any) polypeptides from a host cell, or comprises less than 5, 10, 15, 20, 25, 30, 40, or 50 copies of a polypeptide from a host cell;
   (v) the particle does not comprise a detectable amount of (e.g., any) nucleic acid molecules from a host cell, or comprises less than 2, 3, 4, or 5 copies of a nucleic acid molecule from a host cell;
   (vi) the particle comprises a denaturant in a concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M;
   (vii) does not substantially replicate when introduced into a cell (e.g., a human cell); and/or
   (viii) has a symmetrical morphology.
312. The particle of embodiment 311, wherein the heterologous nucleic acid sequence is about 60-100, 100-200, 200-300, 300-400, 400-500, 500-600, 600-700, 700-800, 800-900, 900-1000, 1000-1500, or 1500-2000, 2000-3000, 3000-4000, or 4000-5000 nucleotides in length.
313. A population of the particles of embodiment 311, wherein at least 75%, 80%, 90%, 95%, 96%, 97%, 98%, 99%, or 100% of the particles in the population comprise at least 50, 55, or 60 copies of an Anellovirus ORF1 molecule.
314. A population of the particles of embodiment 311, wherein at least 90% of the particles in the population have a diameter of at least 30, 31, 32, 33, 34, or 35 nm.
315. A population of the particles of embodiment 311, wherein at least 90%, 95%, 96%, 97%, 98%, 99%, or 100% of the particles have a symmetrical morphology.
316. A population of the particles of embodiment 311, wherein the population does not comprise a detectable amount of polypeptides from a host cell, or comprises less than 1, 2, 3, 4, 5, 10, 15, 20, 25, 30, 40, or 50 copies per particle of a polypeptide from a host cell.
317. A population of the particles of embodiment 311, wherein the population does not comprise a detectable amount of nucleic acid molecules from a host cell, or comprises less than 1, 2, 3, 4, 5, 10, 15, 20, 25, 30, 40, or 50 copies per particle of a nucleic acid molecule from a host cell.
318. A population of the particles of embodiment 311, wherein the population comprises less than 10 ng of nucleic acids.
319. A population of the particles of embodiment 311, wherein the population does not comprise a detectable amount of nucleic acid molecules from a host cell, or comprises less than 1, 2, 3, 4, 5, 10, 15, 20, 25, 30, 40, or 50 copies per particle of a nucleic acid molecule having a length of 200 bp or less from a host cell.
320. A particle comprising:
a proteinaceous exterior comprising about 40-80 (e.g., about 60) copies of an ORF1 molecule; and
wherein the particle:
(i) does not comprise (e.g., does not enclose) a polynucleotide,
(ii) does not comprise (e.g., does not enclose) detectable levels of polynucleotides,
(iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.
321. The particle of embodiment 320, further comprising an exogenous effector.
322. The particle of embodiment 321, wherein the exogenous effector is enclosed within the proteinaceous exterior.
323. The particle of embodiment 320 or 321, wherein the exogenous effector is a polypeptide.
324. The particle of any of embodiments 320-323, wherein the exogenous effector is a small molecule.
325. A composition comprising a plurality of particles, the particles comprising a proteinaceous exterior comprising about 40-80 (e.g., about 60) copies of an ORF1 molecule;
wherein at least 50%, 60%, 70%, 75%, 80%, 85%, 90%, 95%, 96%, 97%, 98%, 99%, 99.5%, or 99.9% of the particles do not comprise (e.g., do not enclose):
   (i) a polynucleotide,
   (ii) a nucleic acid molecule of greater than 1000, 500, 200, or 100 nucleotides in length,
   (iii) a plurality of polynucleotides,
   (iv) a circular nucleic acid molecule,
   (v) a single-stranded nucleic acid molecule, and/or
   (vi) a genetic element (e.g., a genetic element of an anellovector), e.g., as described herein; or
wherein the composition comprises less than 10¹⁰-10¹⁴ (e.g., less than 10¹⁰-10¹¹, 10¹¹-10¹², 10¹²-10¹³, or 10¹³-10¹⁴) viral genome equivalents of nucleic acid molecules (e.g., genetic elements, e.g., of an anellovector as described herein) per kilogram of a subject to be administered the composition (e.g., as determined by qPCR or by measuring optical density).
326. The composition of embodiment 325, further comprising a denaturant (e.g., urea), e.g., in concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M; proteasomes; or ferritin.
327. The composition of embodiment 325 or 326, wherein the composition comprises 0.01-100 mg of the particles (e.g., 0.01-1, 1-10, 10-20, 20-30, 30-40, 40-50, 50-60, 60-70, 70-80, 80-90, or 90-100 mg of the particles).
328. A method of disassembling a particle, the method comprising:
(a) providing a mixture comprising a particle and a denaturant, wherein the particle comprises:
   (i) a proteinaceous exterior comprising a plurality of Anellovirus ORF1 molecules, and
   (ii) a nucleic acid molecule (e.g., a nucleic acid endogenous to a host cell or a nucleic acid exogenous to a host cell, e.g., an anellovirus genome); and
(b) incubating the mixture under conditions suitable for:
   disassembly of the proteinaceous exterior, and
   dissociation of the nucleic acid molecule from the proteinaceous exterior.
329. The method of embodiment 328, wherein the Anellovirus ORF1 molecules were made in mammalian cells.
330. The method of embodiment 328 or 329, wherein the conditions suitable for disassembly of the proteinaceous exterior comprises one or more of: a predetermined conductivity, a detergent (e.g., SDS (e.g., 0.1% SDS), Tween, or Triton), a chaotropic agent (e.g., urea), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving a predetermined temperature.
331. The method of any of embodiments 328-330, wherein the mixture comprises a population of the particles.
332. The method of any of embodiments 328-331, wherein the incubating of (b) results in at least 50%, 60% . . . 95%, or 100% of the population of particles being disassembled.
333. The method of any of embodiments 328-332, further comprising a step of (c) removing (partially or completely) the nucleic acid molecule from the mixture, e.g., by washing.
334. The method of any of embodiments 328-333, wherein the host cell is a human cell.
335. A method of making an anellovector, the method comprising:
(a) providing a mixture comprising a plurality of Anellovirus ORF1 molecules, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to conditions suitable for in vitro assembly of the Anellovirus ORF1 molecules; and
(c) incubating the Anellovirus ORF1 molecules with a plurality of genetic elements, under conditions suitable for assembly of the Anellovirus ORF1 molecules into one or more anellovectors each enclosing one or more of the genetic elements.
336. The method of embodiment 335, wherein the mixture provided in (a) is under denaturing conditions, e.g., wherein the mixture comprises a denaturant at a level sufficient to disassemble a complex (e.g., a proteinaceous exterior) comprising at least about 20, 30, 40, 50, or 60 copies, or 20-30, 30-40, 40-50, or 50-60 copies, of the Anellovirus ORF1 molecule.
337. The method of embodiment 335 or 336, wherein the conditions suitable for in vitro assembly comprise reducing the concentration of a denaturant or removing the mixture from denaturing conditions.
338. A method of making an anellovector, the method comprising:
(a) providing a mixture comprising a plurality of Anellovirus ORF1 molecules and subjecting the mixture to denaturing conditions (e.g., providing a denaturant as part of the mixture, e.g., contacting the mixture with a denaturant), wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to non-denaturing conditions (e.g., reducing the concentration of the denaturant to a level) suitable for in vitro assembly of the Anellovirus ORF1 molecules (e.g., by dialysis); and
(c) incubating the Anellovirus ORF1 molecules with a plurality of genetic elements, under conditions suitable for assembly of the Anellovirus ORF1 molecules into one or more anellovectors each enclosing one or more of the genetic elements.
339. The method of embodiment 338, wherein (b) and (c) are performed concurrently.
340. The method of embodiment 338, wherein (b) is performed prior to (c).
341. The method of any of embodiments 338-340, wherein the genetic elements are introduced into a mixture comprising the Anellovirus ORF1 molecules prior to, concurrently with, or after (b).
342. The method of any of embodiments 338-341, wherein at least 75%, 80%, 90%, 95%, 96%, 97%, 98%, 99%, or 100% of the plurality of ORF1 molecules in the mixture of (a) are comprised in capsomers (e.g., decamers or particles of 25-40 nm in diameter, e.g., 25-30, 30-32, 32-35, or 35-40 nm in diameter or about 25, 26, 27, 28, 29, 30, 31, 32, 33, 34, 35, 36, 37, 38, 39, or 40 nm).
343. The method of any of embodiments 338-342, wherein the ratio of ORF1 molecules in the mixture of (a) comprised in capsomers (e.g., decamers) compared to ORF1 molecules in the mixture of (a) comprised in particles is at least 2:1, 3:1, 4:1, 5:1, 10:1, 50:1, 100:1, 500:1, 1000:1, 5000:1, or 10,000:1.
344. The method of any of embodiments 338-343, wherein at least 75%, 80%, 90%, 95%, 96%, 97%, 98%, 99%, or 100% of the plurality of ORF1 molecules after the incubating of (c) are comprised in anellovectors (e.g., 60 mers or particles of at least 30, 31, 32, 33, 34, or 35 nm in diameter).
345. The method of any of embodiments 338-344, wherein the genetic element encodes an exogenous effector.
346. The method of any of embodiments 338-345, wherein the genetic element is an oligonucleotide.
347. The method of any of embodiments 338-346, wherein the genetic element does not encode a polypeptide or functional nucleic acid.
348. The method of any of embodiments 338-347, wherein the concentration of the denaturant after step (b) is no more than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.
349. The method of any of embodiments 338-248, wherein, after the incubating of (c), at least 75%, 80%, 90%, 95%, 96%, 97%, 98%, 99%, or 100% of the particles in the mixture comprise at least 50, 55, or 60 copies of an Anellovirus ORF1 molecule.
350. The method of any of embodiments 338-349, wherein, after the incubating of (c), at least 90% of the particles in the mixture have a diameter of at least 30, 31, 32, 33, 34, or 35 nm.
351. The method of any of embodiments 338-350, wherein, after the incubating of (c), at least 90%, 95%, 96%, 97%, 98%, 99%, or 100% of the particles in the mixture have a symmetrical morphology.
352. The method of any of embodiments 338-351, wherein the denaturant is selected from a chaotropic agent (e.g., urea), heat (e.g., temperature above about 85, 86, 87, 88, 89, 90, 91, 92, 93, 94, or 95° C.), or pH (e.g., acidic pH or basic pH).
353. A method of making an anelloVLP, the method comprising:
(a) providing a mixture comprising a plurality of Anellovirus ORF1 molecules, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to conditions suitable for in vitro assembly of the Anellovirus ORF1 molecules; and
(c) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.
354. The method of embodiment 353, wherein the mixture provided in (a) is under denaturing conditions, e.g., wherein the mixture comprises a denaturant at a level sufficient to disassemble a complex (e.g., a proteinaceous exterior) comprising at least about 20, 30, 40, 50, or 60 copies, or 20-30, 30-40, 40-50, or 50-60 copies, of the Anellovirus ORF1 molecule.
355. The method of embodiment 353 or 354, wherein the conditions suitable for in vitro assembly comprise reducing the concentration of a denaturant or removing the mixture from denaturing conditions.
356. A method of making an anelloVLP, the method comprising:
(a) providing a mixture comprising a plurality of Anellovirus ORF1 molecules and a denaturant, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) reducing the concentration of the denaturant to a level suitable for in vitro assembly of the Anellovirus ORF1 molecules; and
(c) incubating the Anellovirus ORF1 molecules with a plurality of effectors (e.g., exogenous effectors), under conditions suitable for in vitro assembly of the Anellovirus ORF1 molecules into one or more anelloVLPs each enclosing one or more of the effectors.
357. The method of embodiment 356, wherein the effectors are introduced into a mixture comprising the Anellovirus ORF1 molecules prior to, concurrently with, or after (b).
358. The method of embodiment 356 or 357, wherein at least 75%, 80%, 90%, 95%, 96%, 97%, 98%, 99%, or 100% of the plurality of ORF1 molecules in the mixture of (a) are comprised in capsomers (e.g., decamers or particles of at most 25-40 nm in diameter, e.g., 25-30, 30-32, 32-35, or 35-40 nm in diameter or about 25, 26, 27, 28, 29, 30, 31, 32, 33, 34, 35, 36, 37, 38, 39, or 40 nm).
359. The method of any of embodiments 356-358, wherein the ratio of ORF1 molecules in the mixture of (a) comprised in capsomers (e.g., decamers) compared to ORF1 molecules in the mixture of (a) comprised in particles is at least 2:1, 3:1, 4:1, 5:1, 10:1, 50:1, 100:1, 500:1, 1000:1, 5000:1, or 10,000:1.
360. The method of any of embodiments 356-359, wherein at least 75%, 80%, 90%, 95%, 96%, 97%, 98%, 99%, or 100% of the plurality of ORF1 molecules after the incubating of (c) are comprised in anelloVLPs (e.g., 60 mers or particles of at least 30, 31, 32, 33, 34, or 35 nm in diameter).
361. The method of any of embodiments 356-360, wherein the anelloVLP has one or more of the following characteristics:
(i) does not comprise (e.g., does not enclose) a polynucleotide,
(ii) does not comprise (e.g., does not enclose) detectable levels of polynucleotides,
(iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.
362. The method of any of embodiments 356-361, wherein the concentration of the denaturant after step (b) is no more than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.
363. The method of any of embodiments 356-362, wherein, after the incubating of (c), at least 75%, 80%, 90%, 95%, 96%, 97%, 98%, 99%, or 100% of the particles in the mixture comprise at least 50, 55, or 60 copies of an Anellovirus ORF1 molecule.
364. The method of any of embodiments 356-363, wherein, after the incubating of (c), at least 90% of the particles in the mixture have a diameter of at least 30, 31, 32, 33, 34, or 35 nm.
365. The method of any of embodiments 356-364, wherein, after the incubating of (c), at least 90%, 95%, 96%, 97%, 98%, 99%, or 100% of the particles in the mixture have a symmetrical morphology.
366. The method of any of embodiments 356-365, wherein the denaturant is selected from a chaotropic agent (e.g., urea), heat (e.g., temperature above about 85, 86, 87, 88, 89, 90, 91, 92, 93, 94, or 95° C.), or pH (e.g., acidic pH or basic pH).
367. A method of making an anelloVLP, the method comprising:
(a) providing a mixture comprising a particle and a denaturant, wherein the particle comprises:
   (i) a proteinaceous exterior comprising a plurality of Anellovirus ORF1 molecules, and
   (ii) a nucleic acid molecule (e.g., a host cell nucleic acid molecule); and
(b) incubating the mixture under conditions suitable for:
   disassembly of the proteinaceous exterior, and
   dissociation of the nucleic acid molecule from the proteinaceous exterior;
(c) providing a mixture comprising a plurality of Anellovirus ORF1 molecules and a denaturant, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(d) reducing the concentration of the denaturant to a level suitable for in vitro assembly of the Anellovirus ORF1 molecules; and
(e) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.
368. A polypeptide, e.g., an ORF1 molecule, comprising one or more of:
(a) a first region comprising an amino acid sequence having at least 70% (e.g., at least about 70, 80, 90, 95, 96, 97, 98, 99, or 100%) sequence identity to an arginine-rich region sequence described herein (e.g., MPYYYRRRRYNYRRPRWYGRGWIRRPFRRRFRRKRRVR (SEQ ID NO: 216) or MAWGWWKRRRRWWFRKRWTRGRLRRRWPRSARRRPRRRRVRRRRRWRRGRRKTRTYRRRR RFRRRGRK (SEQ ID NO: 186), or as listed in any one of Tables A1-A32) or a sequence of at least about 40 amino acids comprising at least 60%, 70%, or 80% basic residues (e.g., arginine, lysine, or a combination thereof),
(b) a second region comprising an amino acid sequence having at least 30% (e.g., at least about 30, 35, 40, 50, 60, 70, 80, 90, 95, 96, 97, 98, 99, or 100%) sequence identity to a jelly-roll region sequence described herein (e.g., PTYTTIPLKQWQPPYKRTCYIKGQDCLIYYSNLRLGMNSTMYEKSIVPVHWPGGGSFSVSMLTLD ALYDIHKLCRNWWTSTNQDLPLVRYKGCKITFYQSTFTDYIVRIHTELPANSNKLTYPNTHPLM MMMSKYKHIIPSRQTRRKKKPYTKIFVKPPPQFENKWYFATDLYKIPLLQIHCTACNLQNPFVKP DKLSNNVTLWSLNT (SEQ ID NO: 217), or as listed in any of any one of Tables A1-A32) or a sequence comprising at least 6 (e.g., at least 6, 7, 8, 9, 10, 11, or 12) beta strands;
(c) a third region comprising an amino acid sequence having at least 30% (e.g., at least about 30, 35, 40, 50, 60, 70, 80, 90, 95, 96, 97, 98, 99, or 100%) sequence identity to an N22 domain sequence described herein (e.g., TMALTPFNEPIFTQIQYNPDRDTGEDTQLYLLSNATGTGWDPPGIPELILEGFPLWLIYWGFADFQ KNLKKVTNIDTNYMLVAKTKFTQKPGTFYLVILNDTFVEGNSPYEKQPLPEDNIKWYPQVQYQL EAQNKLLQTGPFTPNIQGQLSDNISMFYKFYFK (SEQ ID NO: 219), or as listed in any of any one of Tables A1-A32); and
(d) a fourth region comprising an amino acid sequence having at least 30% (e.g., at least about 30, 35, 40, 50, 60, 70, 80, 90, 95, 96, 97, 98, 99, or 100%) sequence identity to an Anellovirus ORF1 C-terminal domain (CTD) sequence described herein (e.g., WGGSPPKAINVENPAHQIQYPIPRNEHETTSLQSPGEAPESILYSFDYRHGNYTTTALSRISQDWA LKDTVSKITEPDRQQLLKQALECLQISEETQEKKEKEVQQLISNLRQQQQLYRERIISLLKDQ (SEQ ID NO: 220), or as listed in any of any one of Tables A1-A32);
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).
369. The polypeptide of embodiment 368, wherein the amino acid sequences of the region of (a), (b), (c), and (d) have at least 90% sequence identity to their respective references.
370. The polypeptide of embodiment 368, wherein the polypeptide comprises:
(i) the first region and the second region;
(ii) the first region and the third region;
(iii) the first region and the fourth region;
(iv) the second region and the third region;
(v) the second region and the fourth region;
(vi) the third region and the fourth region;
(vii) the first region, the second region, and the third region;
(viii) the first region, the second region, and the fourth region;
(ix) the first region, the third region, and the fourth region; or
(x) the second region, the third region, and the fourth region.
371. The polypeptide of any of embodiments 368-370, wherein:
the first region comprises an amino acid sequence having at least 70% (e.g., at least about 70, 80, 90, 95, 96, 97, 98, 99, or 100%) sequence identity to an arginine-rich region sequence as listed in any one of Tables A1-A32;
the second region comprises an amino acid sequence having at least 70% (e.g., at least about 70, 80, 90, 95, 96, 97, 98, 99, or 100%) sequence identity to a jelly-roll region sequence as listed in any one of Tables A1-A32;
the third region comprises an amino acid sequence having at least 70% (e.g., at least about 70, 80, 90, 95, 96, 97, 98, 99, or 100%) sequence identity to an N22 domain sequence as listed in any one of Tables A1-A32; and/or
the fourth region comprises an amino acid sequence having at least 70% (e.g., at least about 70, 80, 90, 95, 96, 97, 98, 99, or 100%) sequence identity to a CTD sequence as listed in any one of Tables A1-A32.
372. The polypeptide according to embodiment 371, wherein the amino acid sequences of the first, second, third and fourth region have at least 90% sequence identity to their respective references.
373. The polypeptide of any of the preceding embodiments, wherein the polypeptide comprises, in N-terminal to C-terminal order, the first region, the second region, the third region, and the fourth region.
374. The polypeptide of any of the preceding embodiments, wherein the at least one difference comprises at least one difference in the first region relative to the arginine-rich region of a wild-type ORF1 protein.
375. The polypeptide of any of the preceding embodiments, wherein the first region comprises an arginine-rich region from the ORF1 protein of an Anellovirus other than the wild-type Anellovirus to which the polypeptide, or the portion thereof excluding the first region, has greatest sequence identity.
376. The polypeptide of any of the preceding embodiments, wherein the first region comprises an amino acid sequence having at least 70% sequence identity to the arginine-rich region from an Anellovirus other than the wild-type Anellovirus to which the polypeptide has greatest sequence identity.
377. The polypeptide of any of the preceding embodiments, wherein the second region comprises a jelly-roll region from the ORF1 protein of an Anellovirus other than the wild-type Anellovirus to which the polypeptide, or the portion thereof excluding the second region, has greatest sequence identity.
378. The polypeptide of any of the preceding embodiments, wherein the second region comprises an amino acid sequence having at least 70% sequence identity to the jelly-roll region from an Anellovirus other than the wild-type Anellovirus to which the polypeptide has greatest sequence identity.
379. The polypeptide of any of the preceding embodiments, wherein the third region comprises an N22 domain from the ORF1 protein of an Anellovirus other than the wild-type Anellovirus to which the polypeptide, or the portion thereof excluding the third region, has greatest sequence identity.
380. The polypeptide of any of the preceding embodiments, wherein the third region comprises an amino acid sequence having at least 70% sequence identity to the N22 region from an Anellovirus other than the wild-type Anellovirus to which the polypeptide has greatest sequence identity.
381. The polypeptide of any of the preceding embodiments, wherein the fourth region comprises a CTD domain from the ORF1 protein of an Anellovirus other than the wild-type Anellovirus to which the polypeptide, or the portion thereof excluding the fourth region, has greatest sequence identity.
382. The polypeptide of any of the preceding embodiments, wherein the fourth region comprises an amino acid sequence having at least 70% sequence identity to the CTD region from an Anellovirus other than the wild-type Anellovirus to which the polypeptide has greatest sequence identity.
383. The polypeptide of any of embodiments 368-382, wherein the HVR sequence is positioned between the second region and the third region.
384. The polypeptide of embodiment 383, wherein the HVR sequence comprises an amino acid sequence having at least 70% (e.g., at least about 70, 80, 90, 95, 96, 97, 98, 99, or 100%) sequence identity to the HVR from an Anellovirus other than the wild-type Anellovirus to which the ORF1 protein has greatest sequence identity.
385. The polypeptide of any of embodiment 383 or 384, wherein the HVR sequence is heterologous relative to one or more of the first region, second region, third region, and/or fourth region.
386. The polypeptide of any of embodiments 383-385, wherein the HVR sequence comprises an HVR from the ORF1 protein of an Anellovirus other than the wild-type Anellovirus to which the polypeptide, or the portion thereof excluding the HVR sequence, has greatest sequence identity.
387. The polypeptide of any of embodiments 383-386, wherein the HVR sequence comprises an amino acid sequence having at least 70% sequence identity to the HVR from an Anellovirus other than the wild-type Anellovirus to which the polypeptide has greatest sequence identity.
388. The anellovector of any of the preceding embodiments, wherein the proteinaceous exterior comprises a polypeptide of any of embodiments 368-387.
389. The particle of any of the preceding embodiments, wherein the proteinaceous exterior comprises a polypeptide of any of embodiments 368-387.
390. The anelloVLP of any of the preceding embodiments, wherein the proteinaceous exterior comprises a polypeptide of any of embodiments 368-387.
391. A method of making two or more different Anellovirus ORF molecules, the method comprising:
(i) providing an insect cell comprising a nucleic acid construct encoding two or more different Anellovirus ORF molecules (e.g., two or more of an ORF1, ORF2, ORF2/2, ORF2/3, ORF1/1, and/or ORF1/2 molecule);
(ii) incubating the insect cell under conditions suitable for expression of the two or more different Anellovirus ORF molecules.
392. The method of embodiment 391, wherein the nucleic acid construct comprises sequences encoding all of an ORF1, ORF2, ORF2/2, ORF2/3, ORF1/1, and/or ORF1/2 molecule.
393. The method of embodiment 391, further comprising incubating the insect cell under conditions suitable for secretion of the Anellovirus ORF molecule.
394. The method of embodiment 391, further comprising isolating the Anellovirus ORF molecule from the insect cell.
395. The method of embodiment 394, wherein the isolating step comprises lysing the insect cell.
396. The method of any of embodiments 391-395, wherein the Anellovirus ORF comprises an Anellovirus ORF1 molecule.
397. A method of making an Anellovirus ORF1 molecule, the method comprising:
(i) providing an insect cell comprising a nucleic acid construct encoding an Anellovirus ORF1 molecule, wherein:
   (a) the Anellovirus ORF1 molecule has a molecular weight of at least 101 kDa,
   (b) the Anellovirus ORF1 molecule is a full-length Anellovirus ORF1 protein,
   (c) a plurality of the Anellovirus ORF1 molecules, when in the presence of an Anellovirus genetic element, enclose the Anellovirus genetic element,
   (d) the Anellovirus ORF1 molecule is not a TTV ORF1 protein,
   (e) the Anellovirus ORF1 molecule is a Betatorquevirus or Gammatorquevirus ORF1 molecule; or
   (f) the Anellovirus ORF1 molecule comprises an Anellovirus ORF1 Arginine-rich region and an Anellovirus C-terminal domain;
(ii) incubating the insect cell under conditions suitable for expression of the Anellovirus ORF1 molecule.
398. The method of embodiment 397, further comprising incubating the insect cell under conditions suitable for secretion of the Anellovirus ORF1 molecule.
399. The method of embodiment 397, further isolating the Anellovirus ORF1 molecule from the insect cell.
400. The method of embodiment 399, wherein the isolating step comprises lysing the insect cell.
401. The method of any of the preceding embodiments, wherein the incubation step produces an amount of the Anellovirus ORF1 molecule detectable by Western blot, e.g., as described herein.
402. A method of making an Anellovirus ORF molecule (e.g., an ORF1, ORF2, ORF2/2, ORF2/3, ORF1/1, and/or ORF1/2 molecule), the method comprising:
(i) providing an insect cell (e.g., an Sf9 cell) comprising a nucleic acid construct encoding the Anellovirus ORF molecule;
(ii) incubating the insect cell under conditions suitable for expression of a plurality of the Anellovirus ORF molecules; and
(iii) optionally isolating, purifying, and/or enriching the plurality of Anellovirus ORF molecules from the insect cell or other components or constituents thereof;
thereby making the Anellovirus ORF molecule.
403. The method of embodiment 402, wherein the Anellovirus ORF molecule is fused to a marker (e.g., a His tag), e.g., at its N-terminal end or at its C-terminal end (e.g., as described in Table E1 and/or Example 9).
404. The method of embodiment 402 or 403, wherein the insect cell further comprises a nucleic acid construct encoding one or more additional Anellovirus ORF molecules (e.g., one or more of an ORF1, ORF2, ORF2/2, ORF2/3, ORF1/1, and/or ORF1/2 molecule), and wherein the method further comprises:
incubating the insect cell under conditions suitable for expression of a plurality of the one or more additional Anellovirus ORF molecules, e.g., prior to, concurrently with, or subsequent to step (ii); and
optionally isolating, purifying, and/or enriching the plurality of the one or more additional Anellovirus ORF molecules from the insect cell or other components or constituents thereof, e.g., prior to, concurrently with, or subsequent to step (iii).
405. The method of embodiment 404, wherein the nucleic acid construct encoding the one or more additional Anellovirus ORF molecules is the same as the nucleic acid construct of (i).
406. The method of embodiment 405, wherein the nucleic acid construct of (i) comprises sequences encoding 2, 3, 4, 5, or all 6 of an Anellovirus ORF1, ORF2, ORF2/2, ORF2/3, ORF1/1, and/or ORF1/2 molecule.
407. The method of embodiment 405, wherein the nucleic acid construct of (i) encodes an Anellovirus ORF1, ORF2, ORF2/2, ORF2/3, ORF1/1, and ORF1/2 molecule.
408. The method of embodiment 405, wherein the nucleic acid construct of (i) comprises the full open reading frame region of an Anellovirus genome.
409. The method of embodiment 404, wherein the nucleic acid construct encoding the one or more additional Anellovirus ORF molecules is different from the nucleic acid construct of (i).
410. The method of any of embodiments 404-409, wherein the Anellovirus ORF molecules are from the same Anellovirus genome.
411. The method of any of embodiments 404-409, wherein the Anellovirus ORF molecules are from a plurality of Anellovirus genomes (e.g., wherein the ORF1 molecule is from one Anellovirus genome and the ORF2 molecule is from a different Anellovirus genome).
412. The method of any of embodiments 404-411, wherein one or more of the Anellovirus ORF molecules are from an Alphatorquevirus.
413. The method of any of embodiments 404-412, wherein one or more of the Anellovirus ORF molecules are from a Betatorquevirus.
414. The method of any of embodiments 404-413, wherein one or more of the Anellovirus ORF molecules are from a Gammatorquevirus.
415. The method of any of embodiments 404-414, wherein the nucleic acid construct or constructs each comprises a promoter (e.g., a promoter controlling expression of one or more of the Anellovirus ORF molecules, e.g., a baculovirus polyhedron promoter).
416. The method of any of embodiments 404-415, further comprising incubating the insect cell under conditions suitable for secretion of the Anellovirus ORF molecules.
417. The method of any of embodiments 404-416, wherein the isolating step comprises lysing the insect cell.
418. The method of any of embodiments 404-417, wherein the incubation step produces an amount of the Anellovirus ORF molecule (e.g., ORF1 molecule) detectable by Western blot, e.g., as described herein.
419. The method of any of embodiments 404-418, wherein the incubation step produces at least 1, 2, 3, 4, 5, or 6 mg of the Anellovirus ORF1 molecule per 1 L of cell culture (e.g., Sf9 culture).
420. The method of any of the preceding embodiments, wherein the Anellovirus ORF molecules are isolated, purified, or enriched by isopycnic centrifugation.
421. The method of any of the preceding embodiments, wherein the Anellovirus ORF molecule is an Anellovirus ORF1 molecule, and wherein the method further comprises:
contacting, in vitro, the isolated, purified, or enriched Anellovirus ORF1 molecule with a genetic element under conditions suitable for enclosure of the genetic element by a proteinaceous exterior comprising the Anellovirus ORF1 molecule, e.g., as described herein.
422. An Anellovector genetic element comprising:
a) a recombinase hybrid site;
b) one or both of an Anellovirus 5′ NCR sequence and Anellovirus 3′ NCR sequence, and
c) a sequence encoding an exogenous effector.
423. An Anellovector genetic element comprising:
a) a recombinase hybrid site;
b) an Anellovirus NCR, and
c) a sequence encoding an exogenous effector.
424. The Anellovector genetic element of embodiment 422 or 423, which does not comprise a vector backbone.
425. The Anellovector genetic element of any of the preceding embodiments, which is less than 5 kb, 4 kb, or 3 kb in length.
426. The Anellovector genetic element of any of the preceding embodiments, which comprises, from upstream to downstream, the recombinase hybrid site, the Anellovirus 3′ NCR sequence, the Anellovirus 5′ NCR sequence, and the sequence encoding an exogenous effector.
427. The Anellovector genetic element of any of the preceding embodiments, wherein the recombinase hybrid site is not situated between the Anellovirus 5′ NCR sequence and the Anellovirus 3′ NCR sequence.
428. The Anellovector genetic element of any of the preceding embodiments, which comprises, from upstream to downstream, the Anellovirus 3′ NCR sequence, the recombinase hybrid site, the Anellovirus 5′ NCR sequence, and the sequence encoding an exogenous effector.
429. The Anellovector genetic element of any of the preceding embodiments, wherein the recombinase hybrid site is situated between the Anellovirus 5′ NCR sequence and the Anellovirus 3′ NCR sequence.
430. The Anellovector genetic element of any of the preceding embodiments, which comprises, from upstream to downstream, the recombinase hybrid site, the Anellovirus NCR sequence, and the sequence encoding an exogenous effector.
431. The Anellovector genetic element of any of the preceding embodiments, which comprises, from upstream to downstream, the Anellovirus NCR sequence, and the sequence encoding an exogenous effector.
432. The Anellovector genetic element of any of the preceding embodiments, wherein the recombinase hybrid site is a loxP site, an attL site, or an attR site.
433. The Anellovector genetic element of any of the preceding embodiments, wherein the recombinase hybrid site is a loxP site.
434. The Anellovector genetic element of any of the preceding embodiments, wherein the recombinase hybrid site is an attR site.
435. The Anellovector genetic element of any of the preceding embodiments, wherein the recombinase hybrid site has a nucleotide sequence of a recombinase hybrid site as listed in any of Tables V3-V5, or a sequence having at last 80%, 85%, 90%, or 95% identity thereto.
436. The Anellovector genetic element of any of the preceding embodiments, which is circular.
437. The Anellovector genetic element of any of the preceding embodiments, which is single stranded.
438. The Anellovector genetic element of any of the preceding embodiments, which is double stranded.
439. An Anellovector genetic element construct comprising the nucleic acid sequences of b) and c) of an Anellovector genetic element of any of the preceding embodiments.
440. The Anellovector genetic element construct of embodiment 439, which is double-stranded DNA.
441. The Anellovector genetic element construct of any of the preceding embodiments, which further comprises a first recombinase recognition site (e.g., as described herein) and a second recombinase recognition site (e.g., as described herein).
442. The Anellovector genetic element construct of embodiment 441, wherein the first recombinase recognition site and the second recombinase recognition site flank the nucleic acid sequences of b) and c) of the Anellovector genetic element.
443. The Anellovector genetic element construct of any of the preceding embodiments, which further comprises a vector backbone suitable for replication in a host cell (e.g., a mammalian cell or a bacterial cell).
444. The Anellovector genetic element of any of the preceding embodiments, which comprises an Anellovirus origin of replication.
445. The Anellovector genetic element of any of the preceding embodiments, which does not comprise a bacterial origin of replication.
446. A host cell comprising the Anellovector genetic element of any of the preceding embodiments.
447. The host cell of embodiment 446, wherein the host cell is a mammalian cell.
448. The host cell of embodiment 447, wherein the host cell is a bacterial cell.
449. An Anellovector particle comprising the Anellovector genetic element of any of the preceding embodiments, encapsulation in a proteinaceous exterior.
450. The Anellovector particle of embodiment 449, wherein the proteinaceous exterior comprises an ORF1 molecule.
451. A preparation comprising a plurality of the Anellovector genetic elements of any of the preceding embodiments.
452. The preparation of embodiment 451, which further comprises a first DNA construct according to any of the preceding embodiments.
453. The preparation of any of the preceding embodiments, which does not comprise a detectable amount of wild-type Anellovirus genetic elements.
454. The preparation of any of the preceding embodiments, which comprises fewer than 1 wild-type Anellovirus genetic elements per 1000 copies of the Anellovector genetic element.
455. The preparation of any of the preceding embodiments, which does not comprise a detectable amount of wild-type Anellovirus particles.
456. The preparation of any of the preceding embodiments, which comprises fewer than 1 wild-type Anellovirus particles per 1000 copies of particles comprising the Anellovector genetic element.
457. A method of treating a disease or disorder in the subject in need thereof, the method comprising:
administering the Anellovector particle or preparation of any of the preceding embodiments to the subject,
wherein the exogenous effector is expressed in the subject,
thereby treating the disease or disorder in the subject.
458. A first DNA construct (e.g., a genetic element construct) comprising, in order:
a) a first recombinase recognition site (e.g., a loxP site);
b) an Anellovector genetic element sequence comprising a sequence encoding an exogenous effector;
c) a second recombinase recognition site (e.g., a loxP site) in the same orientation as the first recombinase recognition site; and
d) a vector backbone suitable for replication in a host cell (e.g., a mammalian cell or a bacterial cell);
wherein the first and second recombinase recognition sites are recognized by the same site-specific recombinase, and
wherein the DNA construct is circular and double stranded.
459. The first DNA construct of embodiment 458, wherein the site-specific recombinase is a serine recombinase or a tyrosine recombinase.
460. The first DNA construct of any of the preceding embodiments, wherein the site-specific recombinase is a unidirectional recombinase or a bidirectional recombinase.
461. The first DNA construct of any of the preceding embodiments, wherein the first recombinase recognition site has the same nucleic acid sequence as the second recombinase recognition site.
462. The first DNA construct of any of the preceding embodiments, wherein the first recombinase recognition site has a different nucleic acid sequence as the second recombinase recognition site.
463. The first DNA construct of any of the preceding embodiments, wherein the site-specific recombinase comprises a nuclear localization signal (e.g., an SV40 nuclear localization signal).
464. The first DNA construct of any of the preceding embodiments, wherein the site-specific recombinase comprises an epitope tag (e.g., an HA tag, e.g., comprising the amino acid sequence YPYDVPDYA (SEQ ID NO: 2504)).
465. The first DNA construct of any of the preceding embodiments, wherein the first and second recombinase recognition sites are loxP sites (e.g., a loxP, lox66, or lox71 site as listed in Table V3) and the site-specific recombinase is Cre (e.g., comprising the amino acid sequence of Cre, e.g., as listed in Table V2, or an amino acid sequence having at least 75%, 80%, 85%, 90%, 95%, 96%, 97%, 98%, or 99% sequence identity thereto).
466. The first DNA construct of any of the preceding embodiments, wherein the first and second recombinase recognition sites are loxP sites (e.g., a loxP, lox66, or lox71 site as listed in Table V3), and wherein the site specific recombinase further comprises a nuclear localization signal (e.g., an SV40 nuclear localization signal, e.g., as described herein).
467. The first DNA construct of embodiment 466, wherein the site-specific recombinase is SV40-NLS_iCre (e.g., comprising the amino acid sequence of SV40-NLS_iCre as listed in Table V2, or an amino acid sequence having at least 75%, 80%, 85%, 90%, 95%, 96%, 97%, 98%, or 99% sequence identity thereto).
468. The first DNA construct of any of the preceding embodiments, wherein the first recombinase recognition site is an attP site (e.g., as listed Table V4), the second recombinase recognition site is an attB site (e.g., as listed Table V4), and the site-specific recombinase is Bxb1 (e.g., comprising the amino acid sequence of a Bxb1 as listed in Table V2, or an amino acid sequence having at least 75%, 80%, 85%, 90%, 95%, 96%, 97%, 98%, or 99% sequence identity thereto).
469. The first DNA construct of any of the preceding embodiments, wherein the first recombinase recognition site is an attP site (e.g., as listed in Table V4), and wherein the site specific recombinase further comprises a nuclear localization signal (e.g., an SV40 nuclear localization signal);
optionally wherein the site-specific recombinase further comprises an epitope tag (e.g., an HA tag, e.g., comprising the amino acid sequence YPYDVPDYA (SEQ ID NO: 2504).
470. The first DNA construct of embodiment 469, wherein the first recombinase recognition site is an attP site (e.g., as listed Table V4), the second recombinase recognition site is an attB site (e.g., as listed Table V4), and the site-specific recombinase is SV40-NLS-HA_Bxb1 (e.g., comprising the amino acid sequence of SV40-NLS-HA_Bxb1 as listed in Table V2, or an amino acid sequence having at least 75%, 80%, 85%, 90%, 95%, 96%, 97%, 98%, or 99% sequence identity thereto).
471. The first DNA construct of any of the preceding embodiments, wherein the first recombinase recognition site is an attP site (e.g., as listed Table V5), the second recombinase recognition site is an attB site (e.g., as listed Table V5), and the site-specific recombinase is PhiC31 (e.g., comprising the amino acid sequence of a PhiC31 as listed in Table V2, or an amino acid sequence having at least 75%, 80%, 85%, 90%, 95%, 96%, 97%, 98%, or 99% sequence identity thereto).
472. The first DNA construct of any of the preceding embodiments, wherein the first and second recombinase recognition sites are FRT sites (e.g., comprising a nucleic acid sequence as listed in Table 1 of Turan et al. 2011, FASEB J. 25:4088-4107; incorporated by reference herein in its entirety) and the site-specific recombinase is Flp.
473. The first DNA construct of any of the preceding embodiments, wherein the site-specific recombinase is a serine recombinase.
474. The first DNA construct of embodiment 473, wherein the site-specific recombinase is selected from PhiC31, Bxb1, R4, ParA resolvase, Tn3, Gin, and γδ.
475. The first DNA construct of any of the preceding embodiments, wherein the site-specific recombinase is a tyrosine recombinase.
476. The first DNA construct of embodiment 475, wherein the site-specific recombinase is selected from Cre, Dre, Flp, KD, B2, B3, lambda integrase, HKo22, and HP1.
477. The first DNA construct of any of the preceding embodiments, wherein the site-specific recombinase is A118 or (DFC1.
478. The first DNA construct of any of the preceding embodiments, wherein the site-specific recombinase is a ParA resolvase (e.g., comprising the amino acid sequence of ParA as listed in Table V2, or an amino acid sequence having at least 75%, 80%, 85%, 90%, 95%, 96%, 97%, 98%, or 99% sequence identity thereto).
479. The first DNA construct of any of the preceding embodiments, wherein the first recombinase recognition site has a nucleotide sequence of a recombinase recognition site as listed in any of Tables V1, V3-V5, or a sequence having at last 80%, 85%, 90%, or 95% identity thereto.
480. The first DNA construct of any of the preceding embodiments, wherein the second recombinase recognition site has a nucleotide sequence of a recombinase recognition site as listed in any of Tables V1, V3-V5, or a sequence having at last 80%, 85%, 90%, or 95% identity thereto.
481. The first DNA construct of any of the preceding embodiments, wherein the site-specific recombinase has an amino acid sequence of a recombinase as listed in any of V1 or V2, or a sequence having at last 80%, 85%, 90%, or 95% identity thereto.
482. The first DNA construct of any of the preceding embodiments, wherein the Anellovector genetic element comprises a 5′ NCR sequence and a 3′ NCR sequence.
483. The first DNA construct of embodiment 482, wherein the Anellovirus 5′ NCR has the sequence of a wild-type Anellovirus 5′ NCR or comprises at least one sequence difference relative to the wild-type Anellovirus 5′ NCR.
484. The first DNA construct of embodiment 482, wherein the Anellovirus 3′ NCR has the sequence of a wild-type Anellovirus 3′ NCR or comprises at least one sequence difference relative to the wild-type Anellovirus 3′ NCR.
485. The first DNA construct of any of the preceding embodiments, wherein the Anellovector genetic element comprises an Anellovirus NCR sequence.
486. The first DNA construct of embodiment 485, wherein the Anellovirus NCR has the sequence of a wild-type Anellovirus NCR or comprises at least one sequence difference relative to the wild-type Anellovirus NCR.
487. The first DNA construct of embodiment 482, wherein the 5′ NCR is situated between the sequence that encodes the exogenous effector and the 3′ NCR, e.g., wherein the 5′ NCR and 3′ NCR are both upstream of the sequence that encodes the exogenous effector.
488. The first DNA construct of embodiment 482, wherein the sequence that encodes the exogenous effector is situated between the 5′ NCR and the 3′ NCR.
489. The first DNA construct of any of the preceding embodiments, wherein the 3′ NCR comprises a GC-rich region.
490. The first DNA construct of any of the preceding embodiments, wherein the Anellovirus NCR comprises a GC-rich region.
491. The first DNA construct of any of the preceding embodiments, wherein the Anellovector genetic element does not encode any one or more of (e.g., does not comprise any of) an ORF1, ORF2, ORF2/2, ORF2/3, ORF1/1, or ORF1/2 molecule.
492. The first DNA construct of any of the preceding embodiments, wherein the Anellovector genetic element encodes one or more of (e.g., all of) an ORF1, ORF2, ORF2/2, ORF2/3, ORF1/1, or ORF1/2 molecule.
493. The first DNA construct of any of the preceding embodiments, wherein the Anellovector genetic element further comprises a promoter operably linked to the sequence encoding the exogenous effector.
494. The first DNA construct of any of the preceding embodiments, wherein the Anellovector genetic element further comprises a transcription terminator element, e.g., situated downstream of the sequence encoding the exogenous effector
495. The first DNA construct of any of the preceding embodiments, wherein the Anellovector genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.
496. The first DNA construct of any of the preceding embodiments, wherein the Anellovector genetic element has a length less than about 1.5, 2.0, 2.5, 3.0, 3.5, 4.0, 4.5, or 5.0 kb.
497. The first DNA construct of any of the preceding embodiments, wherein the vector backbone comprises one or both of an origin of replication and a selection marker (e.g., an antibiotic resistance marker).
498. The first DNA construct of embodiment 497, wherein the origin of replication is a mammalian origin (e.g., a viral origin of replication from a virus that infects a mammalian cell).
499. The first DNA construct of embodiment 497, wherein the origin of replication is a bacterial origin, wherein optionally the bacterial origin comprises an ColE1, pMB1, pBR322, or pUC.
500. The first DNA construct of any of the preceding embodiments, wherein the vector backbone is at least 1, 2, 3, or 4 kb in length.
501. The first DNA construct of any of the preceding embodiments, wherein the vector backbone has a length of 1-2, 2-3, 3-4 kb, or 4-5 kb in length.
502. The first DNA construct of any of the preceding embodiments, which has a size that does not permit substantial packaging in a proteinaceous exterior comprising a plurality of ORF1 molecules.
503. The first DNA construct of any of the preceding embodiments, which has a size of at least 5, 6, 7, 8, 9, or 10 kb.
504. The first DNA construct of any of the preceding embodiments, which has a size of 5-6 kb, 6-7 kb, 7-8 kb, 8-9 kb, or 9-10 kb.
505. The first DNA construct of any of the preceding embodiments, wherein the vector backbone comprises a sequence encoding the site-specific recombinase.
506. The first DNA construct of any of the preceding embodiments, wherein the vector backbone does not comprise a sequence encoding a site-specific recombinase.
507. A DNA construct (e.g., a second DNA construct) comprising:
   a) one or more of (e.g., 1, 2, or all 3 of): a sequence encoding an ORF1 molecule, a sequence encoding an ORF2 molecule, and a sequence encoding an ORF3 molecule;
   b) optionally a sequence encoding a replication protein (e.g., a large T antigen, or a functional fragment, mutant, or variant thereof); and
   c) an exogenous origin (e.g., an exogenous viral origin, e.g., an SV40, PCV, or AAV origin).
508. The DNA construct of embodiment 507, which comprises the sequence encoding the replication protein (e.g., the large T antigen).
509. The DNA construct of embodiment 507, which does not comprise the sequence encoding the replication protein (e.g., the large T antigen).
510. The DNA construct of any of the preceding embodiments, wherein the exogenous origin of c) is an SV40 origin.
511. The DNA construct of any of the preceding embodiments, wherein the exogenous origin of c) is an PCV origin.
512. The DNA construct of embodiment 511, wherein the replication protein comprises a PCV Rep and/or a PCV Rep'.
513. The DNA construct of any of the preceding embodiments, wherein the exogenous origin of c) is an AAV origin (e.g., an AAV minimal origin).
514. The DNA construct of embodiment 513, wherein the replication protein comprises a AAV Rep.
515. The DNA construct of any of the preceding embodiments, which does not comprise an Anellovirus 5′ NCR sequence, or a portion thereof comprising the sequence of 10-20, 20-30, 30-40 40-50, 50-60, 60-70, 70-80, 80-90, 90-100, 100-125, 125-150, 150-160, 160-70, or 170-200 contiguous nucleotides therefrom.
516. The DNA construct of any of the preceding embodiments, which does not comprise an Anellovirus 3′ NCR sequence, or a portion thereof comprising the sequence of 10-20, 20-30, 30-40 40-50, 50-60, 60-70, 70-80, 80-90, 90-100, 100-125, 125-150, 150-160, 160-70, or 170-200 contiguous nucleotides therefrom.
517. The DNA construct of any of the preceding embodiments, which:
(i) does not comprise an Anellovirus 5′ NCR sequence, or a portion thereof comprising the sequence of 10-20, 20-30, 30-40 40-50, 50-60, 60-70, 70-80, 80-90, 90-100, 100-125, 125-150, 150-160, 160-70, or 170-200 contiguous nucleotides therefrom; and
(ii) does not comprise an Anellovirus 3′ NCR sequence, or a portion thereof comprising the sequence of 10-20, 20-30, 30-40 40-50, 50-60, 60-70, 70-80, 80-90, 90-100, 100-125, 125-150, 150-160, 160-70, or 170-200 contiguous nucleotides therefrom.
518. The DNA construct of any of the preceding embodiments, which does not comprise an Anellovirus NCR sequence, or a portion thereof comprising the sequence of 10-20, 20-30, 30-40 40-50, 50-60, 60-70, 70-80, 80-90, 90-100, 100-125, 125-150, 150-160, 160-70, 170-200, 200-225, 225-250, 250-275, 275-300, 300-350, or 350-400 contiguous nucleotides therefrom.
519. The DNA construct of any of the preceding embodiments, which comprises less than 150, 151, 152, 153, 154, 155, 156, 157, 158, 159, 160, 161, 162, 163, 164, or 165 nucleotides of contiguous nucleic acid sequence of an Anellovirus 5′ NCR.
520. The DNA construct of any of the preceding embodiments, which comprises less than 150, 151, 152, 153, 154, 155, 156, 157, 158, 159, 160, 161, 162, 163, 164, or 165 nucleotides of contiguous nucleic acid sequence of an Anellovirus 3′ NCR.
521. The DNA construct of any of the preceding embodiments, which comprises:
(i) less than 150, 151, 152, 153, 154, 155, 156, 157, 158, 159, 160, 161, 162, 163, 164, or 165 nucleotides of contiguous nucleic acid sequence of an Anellovirus 5′ NCR; and
(ii) less than 150, 151, 152, 153, 154, 155, 156, 157, 158, 159, 160, 161, 162, 163, 164, or 165 nucleotides of contiguous nucleic acid sequence of an Anellovirus 3′ NCR.
522. The DNA construct of any of the preceding embodiments, which further comprises an IRES, e.g., wherein the IRES is situated between a) and b), e.g., wherein the IRES is immediately upstream of the sequence encoding the large T antigen.
523. The DNA construct of embodiment 521 or 522, which further comprises selectable marker, e.g., wherein the selectable marker comprises a resistance marker (e.g., a kanamycin resistance gene).
524. The DNA construct of any of the preceding embodiments, which comprises an expression cassette comprising the sequence encoding the large T antigen and the SV40 origin.
525. The DNA construct of any of the preceding embodiments, which further comprises one or more additional expression cassettes (e.g., 1, 2, 3, 4, 5, or more additional expression cassettes).
526. The DNA construct of any of the preceding embodiments, wherein the sequence encoding the ORF1 molecule, the sequence encoding the ORF2 molecule, and the sequence encoding the ORF3 molecule are part of a single expression cassette.
527. The DNA construct of any of the preceding embodiments, wherein the sequence encoding the ORF2 molecule, and the sequence encoding the ORF3 molecule are part of a single expression cassette, optionally wherein the expression cassette comprises a sequence encoding a nonfunctional ORF1 (e.g., a sequence that comprises a premature stop codon or lacks a start codon relative to a wild-type ORF1 gene).
528. The DNA construct of any of the preceding embodiments, wherein the sequence encoding the ORF1 molecule is part of a first expression cassette and the sequence encoding the ORF2 molecule and the sequence encoding the ORF3 molecule are part of a second expression cassette.
529. The DNA construct of any of the preceding embodiments, which further comprises a second sequence encoding an ORF1 molecule, e.g., wherein the second sequence encoding the ORF1 molecule is part of a separate expression cassette from the expression cassette that comprises the ORF1 molecule of a).
530. The DNA construct of any of the preceding embodiments, which further comprises an expression cassette encoding a transgene, e.g., a site-specific recombinase.
531. The DNA construct of any of the preceding embodiments, wherein the expression cassettes are in the same orientation.
532. The DNA construct of any of the preceding embodiments, wherein one or more of the expression cassettes are in opposite orientations.
533. A system comprising a first DNA construct according to any of the preceding embodiments and a second DNA construct according to any of the preceding embodiments.
534. The system of embodiment 533, which further comprises a third DNA construct encoding the site-specific recombinase.
535. The system of embodiment 534, wherein the site-specific recombinase comprises a nuclear localization signal (e.g., an SV40 nuclear localization signal).
536. The system of any of the preceding embodiments, wherein the site-specific recombinase comprises an epitope tag (e.g., an HA tag, e.g., comprising the amino acid sequence YPYDVPDYA (SEQ ID NO: 2504).
537. The system of any of the preceding embodiments, where the first DNA construct does not comprise any anellovirus sequences of at least 10, 15, 20, 25, 30, 40, 50, 60, 70, 80, 90, 100, 150, 200, 250, 300, 400, 500, 600, 700, 800, 900, or 1000 nucleotides in length that has greater than 50%, 60%, 70%, 75%, 80%, 85%, 90%, 95%, 96%, 97%, 98%, 99%, or 100% sequence identity to any anellovirus sequence of the same length comprised in the second DNA construct.
538. The system of any of the preceding embodiments, wherein the ratio of amount of the first DNA construct to the amount of the second DNA construct is about 1:1:2, 1:1:1.75, 1:1:1.5, 1:1:1.25, 1:1:1, 1:1:0.75, or 1:1:0.5.
539. The system of any of the preceding embodiments, wherein the ratio of amount of the first DNA construct to the amount of the second DNA construct is between 1:1:2 to 1:1:1.75, 1:1:1.75 to 1:1:1.5, 1:1:1.5 to 1:1:1.25, 1:1:1.25 to 1:1:1, 1:1:1 to 1:1:0.75, or 1:1:0.75 to 1:1:0.5.
540. A host cell comprising the DNA construct or system of any of the preceding embodiments.
541. A host cell comprising a particle comprising the Anellovector genetic element of any of the preceding embodiments.
542. The host cell of embodiment 540 or 541, wherein the host cell is a mammalian cell, e.g., a human cell (e.g., a HEK293 cell or a HEK293T cell), e.g., a human T cell, e.g., a human ALL T cell, e.g., a MOLT4 cell.
543. The host cell of embodiment 540 or 541, wherein the host cell is a bacterial cell, e.g., an E. coli cell.
544. The host cell of any of the preceding embodiments, which further comprises the site-specific recombinase that recognizes the first and second recombinase recognition sites.
545. The host cell of any of the preceding embodiments, which further comprises a minicircle comprising the Anellovector genetic element sequence of the DNA construct.
546. The host cell of any of the preceding embodiments, which further comprises a single stranded Anellovector genetic element comprising a sequence of the Anellovector genetic element sequence of the DNA construct.
547. The host cell of embodiment 546, which comprises a particle comprising the single stranded Anellovector genetic element encapsulated by a proteinaceous exterior.
548. The host cell of any of the preceding embodiments, which further comprises one or more polypeptide chosen from an ORF1, ORF2, ORF2/2, ORF2/3, ORF1/1, or ORF1/2 molecule.
549. A method of excising a double stranded DNA comprising a Anellovector genetic element from a vector backbone, the method comprising:
contacting the DNA construct of any of the preceding embodiments with the site-specific recombinase under conditions that allow for recombination between the first recombination site and the second recombination site, thereby excising the Anellovector genetic element from the vector backbone.
550. A method of providing a first circular double stranded DNA comprising an Anellovector genetic element, the method comprising:
contacting the DNA construct of any of the preceding embodiments with the site-specific recombinase under conditions that allow for recombination between the first recombination site and the second recombination site,
thereby providing the first circular DNA comprising the Anellovector genetic element.
551. The method of embodiment any of the preceding embodiments, wherein the Anellovector genetic element is one strand of the double stranded DNA sequence.
552. The method of any of the preceding embodiments, which produces an Anellovector genetic element comprising a recombinase hybrid site.
553. The method of embodiment 552, wherein the recombinase hybrid site has the same sequence as the first or second recombinase recognition site.
554. The method of embodiment 552, wherein the recombinase hybrid site has a different sequence from the first and second recombinase recognition sites.
555. The method of any of the preceding embodiments, which further produces a circular DNA (e.g., a second circular DNA) comprising the vector backbone and a second recombinase hybrid site.
556. The method of embodiment 555, wherein the second recombinase hybrid site has the same sequence as the first or second recombinase recognition site.
557. The method of embodiment 555, wherein the second recombinase hybrid site has a different sequence from the first and second recombinase recognition sites.
558. The method of embodiment 555, wherein the second recombinase hybrid site has a different sequence from first recombinase hybrid site.
559. The method of any of the preceding embodiments, which does not comprise contacting the DNA construct with a ligase.
560. The method of any of the preceding embodiments, which results in at least 30%, 40%, 50%, 60%, 70%, 80%, or 90% of DNA sequence comprising the Anellovector genetic element being excised from the vector backbone.
561. The method of any of the preceding embodiments, which further comprises introducing the DNA construct into a cell.
562. The method of any of the preceding embodiments, which comprises introducing a nucleic acid encoding the site-specific recombinase into the cell (e.g., before, after, or simultaneously with introducing the DNA construct into the cell).
563. The method of any of the preceding embodiments, which is performed in a host cell.
564. The method of embodiment 563, wherein the host cell is a mammalian cell.
565. The method of embodiment 563 or 564, wherein the method further comprises expressing an ORF1 molecule in the cell.
566. The method of any of the preceding embodiments, further comprising incubating the cell under conditions suitable for encapsulation of the Anellovector genetic element in a proteinaceous exterior comprising the ORF1 molecule.
567. The method of any of the preceding embodiments, wherein the host cell is a bacterial cell
568. The method of any of the preceding embodiments, which further comprises isolating the Anellovector genetic element from the cell.
569. The method of any of the preceding embodiments, which is performed ex vivo, e.g., in a purified system.
570. The method of embodiment 569, which is performed in a cell-free system.
571. An Anellovector genetic element produced by the method of any of the preceding embodiments.
572. A method of making an Anellovector particle, the method comprising:
(i) incubating a host cell comprising the DNA construct of any of the preceding embodiments, under conditions suitable for expressing the ORF1 molecule; and
(ii) incubating the host cell under conditions suitable for encapsulation, within a proteinaceous exterior comprising the ORF1 molecule, of an Anellovector genetic element in the host cell.
573. The method of any of the preceding embodiments, wherein the Anellovector genetic element comprises the Anellovector genetic element of any of the preceding embodiments.
574. The method of any of the preceding embodiments, wherein the host cell further comprises the Anellovector genetic element construct of any of the preceding embodiments.
575. The method of any of the preceding embodiments, wherein the host cell further comprises the first DNA construct of any of the preceding embodiments.
576. The method of embodiment 574 or 575,
wherein the host cell further comprises a site-specific recombinase or a nucleic acid encoding the site-specific recombinase, and
wherein the method further comprises (e.g., prior to step (ii), prior to steps (i) and (ii), or concurrently with step (i)) incubating the host cell under conditions suitable for recombination of the Anellovector genetic construct of the first DNA construct by the site-specific recombinase.
577. A method of making an Anellovector particle, the method comprising:
contacting the Anellovector genetic element of any of the preceding embodiments with a plurality of ORF1 molecules in vitro (e.g., in a cell-free system) under conditions suitable for encapsulation of the Anellovector genetic element within a proteinaceous exterior comprising the ORF1 molecules; and
thereby making an Anellovector particle.
578. The method of embodiment 577, further comprising, prior to the contacting, isolating the Anellovector genetic element from a host cell (e.g., a host cell comprising the Anellovector genetic element construct or first DNA construct of any of the preceding embodiments).
579. The method of embodiment 577, further comprising, prior to the contacting, isolating a double stranded DNA minicircle comprising the sequence of the Anellovector genetic element from a host cell (e.g., a host cell comprising the Anellovector genetic element construct or first DNA construct of any of the preceding embodiments).
580. The method of embodiment 579, further comprising, prior to the contacting:
(i) nicking one strand of the double-stranded DNA minicircle, thereby producing a nicked genetic element construct;
(ii) incubating the nicked genetic element construct in the presence of an exonuclease under conditions suitable for digestion of the nicked strand of the genetic element construct; and
(iii) isolating the Anellovector genetic element.
581. The method of any of the preceding embodiments, further comprising, prior to the contacting, isolating one or more of the ORF1 molecules from a host cell (e.g., a host cell comprising the DNA construct of any of the preceding embodiments).
582. A method of making an Anellovector genetic element, the method comprising:
(i) providing a double-stranded Anellovector genetic element construct (e.g., the Anellovector genetic element construct of any of the preceding embodiments or the first DNA construct of any of the preceding embodiments);
(ii) nicking one strand of the double-stranded Anellovector genetic element construct, thereby producing a nicked genetic element construct; and
(iii) incubating the nicked genetic element construct in the presence of an exonuclease (e.g., T7 exonuclease) under conditions suitable for digestion of the nicked strand of the genetic element construct;
thereby producing an Anellovector genetic element.
583. The method of 582, further comprising isolating the Anellovector genetic element.
584. The method of embodiment 582, wherein one strand of the double-stranded Anellovector genetic element construct comprises one or more (e.g., at least 1, 2, 3, 4, 5, 6, 7, 8, 9, or 10) nicking sites.
585. The method of embodiment 584, wherein the one or more nicking sites are Nt.BbvCI nicking sites.
586. The method of embodiment 584 or 585, wherein the nicking sites are separated by about 10-20, 20-30, 30-40, 40-50, 50-60, 60-70, 70-80, 80-90, 90-100, 100-150, 150-200, 200-250, 250-300, 300-400, 400-500, 500-600, 600-700, 700-800, 800-900, 900-1000, 1000-1500, or 1500-2000 nucleotides.
587. The method of embodiment 584 or 585, wherein the nicking sites are separated by at least 10, 20, 30, 40, 50, 60, 70, 80, 90, 100, 200, 300, 400, 500, 600, 700, 800, 900, 1000, 1500, or 2000 nucleotides.
588. The Anellovector genetic element, Anellovector genetic element construct, system, host cell, Anellovector particle, preparation, method, DNA construct of any of the preceding embodiments, wherein the exogenous effector comprises a therapeutic agent for treating a disease or disorder of the eye (e.g., macular degeneration or a retinal disease).
589. The Anellovector genetic element, Anellovector genetic element construct, system, host cell, Anellovector particle, preparation, method, DNA construct of any of the preceding embodiments, wherein the exogenous effector comprises an intracellular polypeptide or an intracellular nucleic acid.
590. The Anellovector genetic element, Anellovector genetic element construct, system, host cell, Anellovector particle, preparation, method, DNA construct of any of the preceding embodiments, wherein the exogenous effector comprises a secreted polypeptide.
591. The Anellovector genetic element, Anellovector genetic element construct, system, host cell, Anellovector particle, preparation, method, DNA construct of any of the preceding embodiments, wherein the exogenous effector comprises a polypeptide that, when mutated, causes a human disease, or a functional variant of said polypeptide.
592. The Anellovector genetic element, Anellovector genetic element construct, system, host cell, Anellovector particle, preparation, method, DNA construct of any of the preceding embodiments, which comprises an Anellovirus 5′ NCR sequence of Ring 2, Ring 19, Ring 20, Ring 1, or Ring 3 (e.g., as described herein), or a nucleic acid sequence having at least 75%, 80%, 85%, 90%, 95%, 96%, 97%, 98%, or 99% sequence identity thereto.
593. The Anellovector genetic element, Anellovector genetic element construct, system, host cell, Anellovector particle, preparation, method, DNA construct of any of the preceding embodiments, which comprises an Anellovirus 3′ NCR sequence of Ring 2, Ring 19, Ring 20, Ring 1, or Ring 3 (e.g., as described herein), or a nucleic acid sequence having at least 75%, 80%, 85%, 90%, 95%, 96%, 97%, 98%, or 99% sequence identity thereto.
594. The Anellovector genetic element, Anellovector genetic element construct, system, host cell, Anellovector particle, preparation, method, DNA construct of any of the preceding embodiments, which comprises an Anellovirus NCR sequence of Ring 2, Ring 19, Ring 20, Ring 1, or Ring 3 (e.g., as described herein), or a nucleic acid sequence having at least 75%, 80%, 85%, 90%, 95%, 96%, 97%, 98%, or 99% sequence identity thereto.
595. The system, host cell, Anellovector particle, preparation, method, DNA construct of any of the preceding embodiments, wherein the proteinaceous exterior comprises an ORF1 molecule comprising the amino acid sequence of an ORF1 protein of Ring 2, Ring 19, Ring 20, Ring 1, or Ring 3 (e.g., as described herein), or an amino acid sequence having at least 75%, 80%, 85%, 90%, 95%, 96%, 97%, 98%, or 99% sequence identity thereto.
596. The Anellovector genetic element, Anellovector genetic element construct, system, host cell, Anellovector particle, preparation, method, DNA construct of any of the preceding embodiments, wherein the Anellovector genetic element comprises a promoter, e.g., upstream of the sequence encoding the exogenous effector.
597. The Anellovector genetic element, Anellovector genetic element construct, system, host cell, Anellovector particle, preparation, method, DNA construct of any of the preceding embodiments, wherein the Anellovirus ORF1 molecule does not comprise an Anellovirus ORF1 structural arginine-rich region (e.g., as described herein) or a sequence having at least 75%, 80%, 85%, 90%, 95%, 96%, 97%, 98%, or 99% sequence identity thereto, e.g., a sequence of at least about 40 amino acids comprising at least 60%, 70%, or 80% basic residues (e.g., arginine, lysine, or a combination thereof).
598. The Anellovector genetic element, Anellovector genetic element construct, system, host cell, Anellovector particle, preparation, method, DNA construct of any of the preceding embodiments, wherein the Anellovirus ORF1 molecule does not comprise an Anellovirus ORF1 structural C-terminal domain (CTD) (e.g., as described herein) or a sequence having at least 75%, 80%, 85%, 90%, 95%, 96%, 97%, 98%, or 99% sequence identity thereto.
599. The Anellovector genetic element, Anellovector genetic element construct, system, host cell, Anellovector particle, preparation, method, DNA construct of any of the preceding embodiments, wherein the Anellovirus ORF1 molecule comprises an Anellovirus ORF1 structural jelly-roll region, e.g., an amino acid sequence having at least 30% (e.g., at least about 30, 35, 40, 50, 60, 70, 80, 90, 95, 96, 97, 98, 99, or 100%) sequence identity to a Anellovirus ORF1 structural jelly-roll region sequence described herein or a sequence comprising at least 6 beta strands.
600. The Anellovector genetic element, Anellovector genetic element construct, system, host cell, Anellovector particle, preparation, method, DNA construct of any of the preceding embodiments, wherein the Anellovirus ORF1 molecule comprises an Anellovirus ORF1 structural N22 domain, e.g., an amino acid sequence having at least 30% (e.g., at least about 30, 35, 40, 50, 60, 70, 80, 90, 95, 96, 97, 98, 99, or 100%) sequence identity to an Anellovirus ORF1 structural N22 domain sequence described herein.
601. The Anellovector genetic element, Anellovector genetic element construct, system, host cell, Anellovector particle, preparation, method, DNA construct of any of the preceding embodiments, wherein the Anellovirus ORF1 molecule comprises an Anellovirus ORF1 structural C-terminal domain (CTD), e.g., an amino acid sequence having at least 30% (e.g., at least about 30, 35, 40, 50, 60, 70, 80, 90, 95, 96, 97, 98, 99, or 100%) sequence identity to an Anellovirus ORF1 CTD sequence described herein.
602. The Anellovector genetic element, Anellovector genetic element construct, system, host cell, Anellovector particle, preparation, method, DNA construct of any of the preceding embodiments, wherein the Anellovirus ORF1 molecule comprises a portion of a structural C-terminal domain (CTD) of an Anellovirus ORF1 molecule, which comprises a deletion of about 20-30, 30-40 (e.g., about 37), 40-50 (e.g., about 55), 50-60, 60-70, 70-80, 80-90, 90-100, 100-110, 110-120, 120-130 (e.g., about 129), 130-140 (e.g., about 131), 140-150 (e.g., about 148), or 150-160 (e.g., about 155) amino acids at the C-terminal end of the structural CTD, relative to a corresponding wild-type structural CTD of the Anellovirus ORF1 molecule.
603. A DNA construct comprising:
   (a) one or both of a sequence encoding an ORF2 molecule, and a sequence encoding an ORF3 molecule; and
   (b) a sequence having at least 50%, 60%, 70%, 75%, 80%, 85%, 90%, 95%, 96%, 97%, 98%, or 99% identity to a wild-type ORF1 gene (e.g., a gene encoding a wild-type ORF1 molecule), wherein the sequence comprises a first premature stop codon relative to the wild-type ORF1 gene.
604. A DNA construct comprising:
   (a) one or both of a sequence encoding an ORF2 molecule, and a sequence encoding an ORF3 molecule; and
   (b) a sequence encoding a nonfunctional ORF1 molecule.
605. The DNA construct of embodiment 603, wherein the sequence having at least 50%, 60%, 70%, 75%, 80%, 85%, 90%, 95%, 96%, 97%, 98%, or 99% identity to a wild-type ORF1 gene further comprises a second premature stop codon.
606. The DNA construct of embodiment 604, wherein the sequence encoding the nonfunctional ORF1 molecule comprises a first premature stop codon.
607. The DNA construct of embodiment 606, wherein the sequence encoding the nonfunctional ORF1 molecule further comprises a second premature stop codon.
608. The DNA construct of embodiment 607, wherein the first premature stop codon and the second premature stop codon are in immediately adjacent codons.
609. The DNA construct of any of embodiments 605-608, wherein the first premature stop codon and/or the second premature stop codon are located within the first 30, 35, 40, 45, 50, 54, 55, 60, or 65 codons of the sequence encoding the nonfunctional ORF1 molecule or the sequence having at least 50%, 60%, 70%, 75%, 80%, 85%, 90%, 95%, 96%, 97%, 98%, or 99% identity to a wild-type ORF1 gene.
610. The DNA construct of any of embodiments 603-609, wherein the sequence encoding the ORF2 molecule and the sequence encoding the ORF1 molecule overlap.
611. The DNA construct of any of embodiments 603-610, wherein the sequence encoding the ORF3 molecule and the sequence encoding the ORF1 molecule overlap.
612. The DNA construct of any of embodiments 603-611, which does not produce a detectable amount of full-length ORF1 molecules.
613. The DNA construct of any of embodiments 603-612, which produces an amount of an ORF2 molecule that is no less than 90%, 95%, 96%, 97%, 98%, or 99% the amount of ORF2 molecule produced by an otherwise identical DNA construct that lacks the premature stop codons.
614. The DNA construct of any of embodiments 603-613, which produces an amount of an ORF3 molecule that is no less than 90%, 95%, 96%, 97%, 98%, or 99% the amount of ORF3 molecule produced by an otherwise identical DNA construct that lacks the premature stop codons.
615. The DNA construct of any of embodiments 603-614, which produces an amount of an ORF1/1 molecule that is no less than 90%, 95%, 96%, 97%, 98%, or 99% the amount of ORF1/1 molecule produced by an otherwise identical DNA construct that lacks the premature stop codons.
616. The DNA construct of any of embodiments 603-615, wherein the DNA construct comprises a sequence encoding a replication protein (e.g., a large T antigen, or a functional fragment, mutant, or variant thereof).
617. The DNA construct of any of embodiments 603-616, wherein the DNA construct comprises an exogenous origin (e.g., an exogenous viral origin, e.g., an SV40, PCV, or AAV origin).
618. The DNA construct of any of embodiments 603 and 605-617, wherein the wild-type ORF1 gene is a Ring2 ORF1 gene or a Ring19 ORF1 gene.
619. The DNA construct of any of embodiments 603-618, wherein the ORF2 molecule comprises the amino acid sequence of a Ring2 ORF2 protein or a Ring19 ORF2 protein, or an amino acid sequence having at least 75%, 80%, 85%, 90%, 95%, 96%, 97%, 98%, or 99% identity thereto.
620. The DNA construct of any of embodiments 603-619, wherein the ORF3 molecule comprises the amino acid sequence of a Ring2 ORF3 protein or a Ring19 ORF3 protein, or an amino acid sequence having at least 75%, 80%, 85%, 90%, 95%, 96%, 97%, 98%, or 99% identity thereto.
621. A system comprising:
   (i) the DNA construct of any of embodiments 603-620; and
   (ii) a DNA construct comprising a sequence encoding an ORF1 molecule.
622. A method of producing an anellovector, the method comprising:
(I) providing a cell comprising:
   (a) the system of embodiment 621;
   (b) a genetic element construct comprising:
      (i) a first recombinase recognition site (e.g., a loxP site);
      (ii) an Anellovector genetic element sequence comprising a sequence encoding an exogenous effector; and
      (iii) a second recombinase recognition site (e.g., a loxP site) in the same orientation as the first recombinase recognition site;
   (c) a construct comprising a sequence encoding a site-specific recombinase;
(II) incubating the cell under conditions suitable for production of an anellovector; thereby producing the anellovector.
623. The method of embodiment 622, wherein the conditions suitable for production of an anellovector comprise:
   (i) conditions suitable for expression of the ORF1 molecule;
   (ii) conditions suitable for expression of the ORF2 and/or ORF3 molecule;
   (iii) conditions suitable for expression of the site-specific recombinase;
   (iv) conditions suitable for excision of the Anellovector genetic element sequence from the genetic element construct; and/or
   (v) conditions suitable for encapsulation of an Anellovector genetic element within a proteinaceous exterior comprising the ORF1 molecule.
624. The method of any of embodiments 622-623, further comprising introducing the system of embodiment M1 into the cell.
625. The method of any of embodiments 622-624, further comprising introducing the genetic element construct into the cell.
626. The method of any of embodiments 622-625, further comprising introducing the construct comprising the sequence encoding the site-specific recombinase into the cell.
627. The method of any of embodiments 622-626, wherein the anellovector comprises a genetic element and a proteinaceous exterior encapsulating the genetic element.
628. The method of embodiment 627, wherein the genetic element is a DNase-protected genetic element.
629. A DNA construct comprising one or more of (e.g., 1, 2, or all 3 of): a sequence encoding an ORF1 molecule, a sequence encoding an ORF2 molecule, and a sequence encoding an ORF3 molecule; and
wherein the DNA construct does not comprise an Anellovirus non-coding region (NCR) sequence, or a portion thereof comprising the sequence of 10-20, 20-30, 30-40 40-50, 50-60, 60-70, 70-80, 80-90, 90-100, 100-125, 125-150, 150-160, 160-70, 170-200, 200-225, 225-250, 250-275, 275-300, 300-350, or 350-400 contiguous nucleotides therefrom.
630. The DNA construct of embodiment 629, wherein the first intron of the Anellovirus NCR is absent.
631. The DNA construct of any of embodiments 629-630, which comprises the first exon of the Anellovirus NCR.
632. The DNA construct of any of embodiments 629-630, wherein the DNA construct comprises the Kozak sequence and excludes the remaining portion of the first exon of the Anellovirus NCR.
633. The DNA construct of any of embodiments 629-632, wherein nucleotides 1682-1769 of SEQ ID NO: 2000 are absent.
634. The DNA construct of any of embodiments 629-633, which does not comprise any contiguous sequence of nucleotides having more than 50%, 60%, 70%, 75%, 80%, 85%, 90%, 95%, 96%, 97%, 98%, 99%, or 100% sequence identity to a first intron of the Anellovirus NCR, e.g., Intron 1 of pRTx-3525, e.g., nucleotides 1682-1769 of SEQ ID NO: 2000.
635. The DNA construct of any of embodiments 629-634, wherein nucleotides 1625-1675 of SEQ ID NO: 2000 are absent.
636. The DNA construct of any of embodiments 629-635, which does not comprise any contiguous sequence of nucleotides having more than 50%, 60%, 70%, 75%, 80%, 85%, 90%, 95%, 96%, 97%, 98%, 99%, or 100% sequence identity to a portion of a first exon of the Anellovirus NCR (e.g., Exon 1 of pRTx-3525, e.g., nucleotides 1625-1675 of the SEQ ID NO: 2000), optionally wherein the DNA construct comprises a Kozak sequence and excludes the remaining portion of the first exon.
637 The DNA construct of embodiment 636, which does not comprise any contiguous sequence of nucleotides having more than 50%, 60%, 70%, 75%, 80%, 85%, 90%, 95%, 96%, 97%, 98%, 99%, or 100% sequence identity to nucleotides 1625-1675 of SEQ ID NO: 2000.
638. The DNA construct of any of embodiments 629-637, which comprises a nucleic acid sequence having at least 50%, 60%, 70%, 75%, 80%, 85%, 90%, 95%, 96%, 97%, 98%, 99%, or 100% sequence identity to a portion of a first exon of the Anellovirus NCR (e.g., Exon 1 of pRTx-3525, e.g., nucleotides 1625-1681 of the SEQ ID NO: 2000).
639. The DNA construct of embodiment 638, which comprises a nucleic acid sequence having at least 50%, 60%, 70%, 75%, 80%, 85%, 90%, 95%, 96%, 97%, 98%, 99%, or 100% sequence identity to nucleotides 1625-1675 of SEQ ID NO: 2000.
640. The DNA construct of any of embodiments 629-639, which does not comprise:
   (i) any contiguous sequence of nucleotides having more than 50%, 60%, 70%, 75%, 80%, 85%, 90%, 95%, 96%, 97%, 98%, 99%, or 100% sequence identity to a first intron of the Anellovirus NCR, e.g., Intron 1 of pRTx-3525, e.g., nucleotides 1682-1769 of SEQ ID NO: 2000; and
   (ii) any contiguous sequence of nucleotides having more than 50%, 60%, 70%, 75%, 80%, 85%, 90%, 95%, 96%, 97%, 98%, 99%, or 100% sequence identity to nucleotides 1625-1675 of SEQ ID NO: 2000.
641 The DNA construct of any of embodiments 629-640, wherein nucleotides 1682-1769 and 1625-1675 of SEQ ID NO: 2000 are absent.
642. The DNA construct of any of embodiments 629-641, which:
   (i) does not comprise any contiguous sequence of nucleotides having more than 50%, 60%, 70%, 75%, 80%, 85%, 90%, 95%, 96%, 97%, 98%, 99%, or 100% sequence identity to a first intron of the Anellovirus NCR, e.g., Intron 1 of pRTx-3525, e.g., nucleotides 1682-1769 of SEQ ID NO: 2000; and
   (ii) comprises a contiguous sequence of nucleotides having at least 50%, 60%, 70%, 75%, 80%, 85%, 90%, 95%, 96%, 97%, 98%, 99%, or 100% sequence identity to nucleotides 1625-1675 of SEQ ID NO: 2000.
643. The DNA construct of any of embodiments 629-642, which comprises nucleotides 1625-1675 of SEQ ID NO: 2000, and which does not comprise nucleotides 1682-1769 of SEQ ID NO: 2000.
644. The DNA construct of any of embodiments 629-643, which comprises a Kozak sequence, e.g., a Kozak sequence native to the NCR (e.g., a Kozak sequence having at least 75%, 80%, 85%, 90%, 95%, 96%, 97%, 98%, 99%, or 100% sequence identity to nucleotides 1676-1681 of SEQ ID NO: 2000).
645. A DNA construct comprising:
   (a) one or more of (e.g., 1, 2, or all 3 of): a sequence encoding an ORF1 molecule, a sequence encoding an ORF2 molecule, and a sequence encoding an ORF3 molecule; and
   (b) a T cell promoter operably linked to one or more (e.g., 1, 2, or all 3 of) sequences of (a).
646. The DNA construct of embodiment 645, wherein the T cell promoter is selected from the group consisting of an MNDU3, SFFV, MSCV, Distal Lck (dLck), CD3δ, EF1a, RPBSA, and Cbh promoter.
647. The DNA construct of embodiment 645, wherein the T cell promoter is an MNDU3 promoter.
648. The DNA construct of embodiment 647, wherein the MNDU3 promoter comprises at least 75%, 80%, 85%, 90%, 95%, 96%, 97%, 98%, 99%, or 100% sequence identity to the nucleic acid sequence of SEQ ID NO: 2784.
649. The DNA construct of embodiment 645, wherein the T cell promoter is a SFFV promoter.
650. The DNA construct of embodiment 649, wherein the SFFV promoter comprises at least 75%, 80%, 85%, 90%, 95%, 96%, 97%, 98%, 99%, or 100% sequence identity to the nucleic acid sequence of SEQ ID NO: 2786.
651. The DNA construct of embodiment 645, wherein the T cell promoter is a MSCV promoter.
652. The DNA construct of embodiment 651, wherein the MSCV promoter comprises at least 75%, 80%, 85%, 90%, 95%, 96%, 97%, 98%, 99%, or 100% sequence identity to the nucleic acid sequence of SEQ ID NO: 2785.
653. The DNA construct of any of embodiments 645-652, wherein the DNA construct comprises a sequence encoding a replication protein (e.g., a large T antigen, or a functional fragment, mutant, or variant thereof).
654. The DNA construct of any of embodiments 645-653, wherein the DNA construct comprises an exogenous origin (e.g., an exogenous viral origin, e.g., an SV40, PCV, or AAV origin).
655. The DNA construct of any of embodiments 645-654, wherein the ORF1 molecule comprises the amino acid sequence of a Ring2 ORF1 protein or a Ring19 ORF1 protein, or an amino acid sequence having at least 75%, 80%, 85%, 90%, 95%, 96%, 97%, 98%, or 99% identity thereto.
656. The DNA construct of any of embodiments 645-655, wherein the ORF2 molecule comprises the amino acid sequence of a Ring2 ORF2 protein or a Ring19 ORF2 protein, or an amino acid sequence having at least 75%, 80%, 85%, 90%, 95%, 96%, 97%, 98%, or 99% identity thereto.
657. The DNA construct of any of embodiments 645-656, wherein the ORF3 molecule comprises the amino acid sequence of a Ring2 ORF3 protein or a Ring19 ORF3 protein, or an amino acid sequence having at least 75%, 80%, 85%, 90%, 95%, 96%, 97%, 98%, or 99% identity thereto.
658. A system comprising:
   (i) the DNA construct of any of embodiments 645-657; and
   (ii) a DNA construct comprising a sequence encoding an ORF1 molecule.
659. A method of producing an anellovector, the method comprising:
(I) providing a cell comprising:
   (a) the system of embodiment 658;
   (b) a genetic element construct comprising:
      (i) a first recombinase recognition site (e.g., a loxP site);
      (ii) an Anellovector genetic element sequence comprising a sequence encoding an exogenous effector; and
      (iii) a second recombinase recognition site (e.g., a loxP site) in the same orientation as the first recombinase recognition site;
   (c) a construct comprising a sequence encoding a site-specific recombinase;
(II) incubating the cell under conditions suitable for production of an anellovector;
thereby producing the anellovector.
660. The method of embodiment 659, wherein the anellovector comprises a genetic element and a proteinaceous exterior encapsulating the genetic element.
661. The method of embodiment 660, wherein the genetic element is a DNase-protected genetic element.
662. An anellovector comprising:
(i) a proteinaceous exterior comprising an ORF1 molecule, wherein the ORF1 molecule has a higher level of amino acid sequence similarity to the sequence of an ORF1 molecule encoded by a first Anellovirus genomic sequence than to an ORF1 molecule encoded by a second Anellovirus genomic sequence, and
(ii) a genetic element enclosed by the proteinaceous exterior, wherein the genetic element comprises:
a nucleic acid sequence encoding an exogenous effector; and
one or both of:
(a) an Anellovirus GC-rich region, wherein the Anellovirus GC-rich region has a higher level of sequence similarity to the Anellovirus GC-rich region of the second Anellovirus genomic sequence than to the Anellovirus GC-rich region of the first Anellovirus genomic sequence; and
(b) an Anellovirus 5′ UTR conserved domain, wherein the Anellovirus 5′ UTR conserved domain has a higher level of sequence similarity to the Anellovirus 5′ UTR conserved domain of the second Anellovirus genomic sequence than to the Anellovirus 5′ UTR conserved domain of the first Anellovirus genomic sequence.
663. An anellovector comprising:
(i) a proteinaceous exterior comprising an ORF1 molecule, having an ORF1 sequence of a first Anellovirus genomic sequence, or a polypeptide comprising an amino acid sequence having at least about 70%, 75%, 80%, 85%, 90%, 95%, 96%, 97%, 98%, or 99% sequence identity thereto; and
(ii) a genetic element enclosed by the proteinaceous exterior, wherein the genetic element comprises:
a nucleic acid sequence encoding an exogenous effector; and
one or both of:
(a) an Anellovirus GC-rich region, having the sequence of an Anellovirus GC-rich region of a second Anellovirus genomic sequence, or a nucleic acid comprising a nucleotide sequence having at least about 85%, 90%, 95%, 96%, 97%, 98%, or 99% sequence identity thereto,
wherein the Anellovirus GC-rich region has a higher level of sequence similarity to the Anellovirus GC-rich region of the second Anellovirus genomic sequence than to the Anellovirus GC-rich region of the first Anellovirus genomic sequence; and
(b) an Anellovirus 5′ UTR conserved domain, having the sequence of an Anellovirus 5′ UTR conserved domain of the second Anellovirus genomic sequence, or a nucleic acid comprising a nucleotide sequence having at least about 91%, 92%, 93%, 94%, 95%, 96%, 97%, 98%, or 99% sequence identity thereto,
wherein the Anellovirus 5′ UTR conserved domain has a higher level of sequence similarity to the Anellovirus 5′ UTR conserved domain of the second Anellovirus genomic sequence than to the Anellovirus 5′ UTR conserved domain of the first Anellovirus genomic sequence.
664. An anellovector comprising:
(i) a proteinaceous exterior comprising an ORF1 molecule derived from a first Anellovirus genomic sequence, and
(ii) a genetic element enclosed by the proteinaceous exterior, wherein the genetic element comprises:
a promoter element operably linked to a nucleic acid sequence encoding an exogenous effector; and
one or both of:
(a) an Anellovirus GC-rich region derived from a second Anellovirus genomic sequence, and
(b) an Anellovirus 5′ UTR conserved domain derived from the second Anellovirus genomic sequence.
665. The anellovector of any of the preceding embodiments, wherein the first Anellovirus genomic sequence is the genomic sequence of a Betatorquevirus.
666. The anellovector of any of the preceding embodiments, wherein the second Anellovirus genomic sequence is the genomic sequence of a Betatorquevirus.
667. The anellovector of any of the preceding embodiments, wherein the first Anellovirus genomic sequence is the genomic sequence of an Alphatorquevirus.
668. The anellovector of any of the preceding embodiments, wherein the second Anellovirus genomic sequence is the genomic sequence of an Alphatorquevirus.
669. The anellovector of any of the preceding embodiments, wherein the first Anellovirus genomic sequence is the genomic sequence of a Gammatorquevirus.
670. The anellovector of any of the preceding embodiments, wherein the second Anellovirus genomic sequence is the genomic sequence of a Gammatorquevirus.
671. The anellovector of any of embodiments 662-666, 668, or 670, wherein the first Anellovirus genomic sequence is a Ring19 Anellovirus having a genome sequence of SEQ ID NO: 1.
672. The anellovector of any of embodiments 662-666, 668, or 670, wherein the first Anellovirus genomic sequence is a Ring2 Anellovirus having a genome sequence of SEQ ID NO: 54.
673. The anellovector of any of embodiments 662-666, 668, or 670, wherein the first Anellovirus genomic sequence is a Ring9 Anellovirus having a genome sequence of SEQ ID NO: 1001.
674. The anellovector of any of embodiments 662-666, 668, or 670, wherein the first Anellovirus genomic sequence is a Ring25 Anellovirus having a genome sequence of SEQ ID NO: 210.
675. The anellovector of any of embodiments 662-666, 668, or 670, wherein the first Anellovirus genomic sequence is a Ring34 Anellovirus having a genome sequence of SEQ ID NO: 207.
676. The anellovector of any of embodiments 662-666, 668, or 670, wherein the first Anellovirus genomic sequence is a Ring161 Anellovirus having a genome sequence of SEQ ID NO: 204.
677. The anellovector of any of embodiments 662-666, 668, or 670, wherein the first Anellovirus genomic sequence is a YYBYFW Anellovirus having a genome sequence of SEQ ID NO: 200.
678. The anellovector of any of embodiments 662-666, 668, or 670, wherein the first Anellovirus genomic sequence is a Ring10 Anellovirus having a genome sequence of SEQ ID NO: 1008.
679. The anellovector of any of embodiments 662-666, 667, 669, 671, or 673-678, wherein the second Anellovirus genomic sequence is a Ring2 Anellovirus having a genome sequence of SEQ ID NO: 54.
680. The anellovector of any of embodiments 662-666, 667, 669, or 672-678, wherein the second Anellovirus genomic sequence is a Ring19 Anellovirus having a genome sequence of SEQ ID NO: 1.
681. The anellovector of any of embodiments 662-666, 667, 669, 671, 672, or 674-678, wherein the second Anellovirus genomic sequence is a Ring9 Anellovirus having a genome sequence of SEQ ID NO: 1001.
682. The anellovector of any of embodiments 662-666, 667, 669, 671-673, or 675-678, wherein the second Anellovirus genomic sequence is a Ring25 Anellovirus having a genome sequence of SEQ ID NO: 210.
683. The anellovector of any of embodiments 662-666, 667, 669, 671-674, or 676-678, wherein the second Anellovirus genomic sequence is a Ring34 Anellovirus having a genome sequence of SEQ ID NO: 207.
684. The anellovector of any of embodiments 662-666, 667, 669, 671-675, or 677-678, wherein the second Anellovirus genomic sequence is a Ring161 Anellovirus having a genome sequence of SEQ ID NO: 204.
685. The anellovector of any of embodiments 662-666, 667, 669, 671-676, or 678, wherein the second Anellovirus genomic sequence is a YYBYFW Anellovirus having a genome sequence of SEQ ID NO: 200.
686. The anellovector of any of embodiments 662-666, 667, 669, or 671-677, wherein the second Anellovirus genomic sequence is a Ring10 Anellovirus having a genome sequence of SEQ ID NO: 1008.
687. The anellovector of any of embodiments 662-666, wherein the second Anellovirus genomic sequence is a Ring19 Anellovirus having a genome sequence of SEQ ID NO: 1 and:
a) the first Anellovirus genomic sequence is a Ring2 Anellovirus having a genome sequence of SEQ ID NO: 54;
b) the first Anellovirus genomic sequence is a Ring9 Anellovirus having a genome sequence of SEQ ID NO: 1001;
c) the first Anellovirus genomic sequence is a Ring25 Anellovirus having a genome sequence of SEQ ID NO: 210;
d) the first Anellovirus genomic sequence is a Ring34 Anellovirus having a genome sequence of SEQ ID NO: 207;
e) the first Anellovirus genomic sequence is a Ring161 Anellovirus having a genome sequence of SEQ ID NO: 204;
f) the first Anellovirus genomic sequence is a YYBYFW Anellovirus having a genome sequence of SEQ ID NO: 200; or
g) the first Anellovirus genomic sequence is a Ring10 Anellovirus having a genome sequence of SEQ ID NO: 1008.
688. The anellovector of any of embodiments 662-666, 668, or 670, wherein the ORF1 molecule has a sequence according to any of Tables A1-A32, or a polypeptide comprising an amino acid sequence having at least about 70%, 75%, 80%, 85%, 90%, 95%, 96%, 97%, 98%, or 99% sequence identity thereto.
689. The anellovector of any of embodiments 662-666, 667, or 669, wherein the Anellovirus GC-rich region has a sequence of the reverse complement of the GC-rich region of any of Tables N1-N32, or functional fragment thereof, or a nucleic acid sequence having at least about 70%, 75%, 80%, 85%, 90%, 95%, 96%, 97%, 98%, or 99% sequence identity thereto.
690. The anellovector of any of embodiments 662-666, wherein:
a) the ORF1 molecule has a sequence according to SEQ ID NO: 58, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto;
b) the ORF1 molecule has a sequence according to SEQ ID NO: 1005, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto;
c) the ORF1 molecule has a sequence according to SEQ ID NO: 211, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto;
d) the ORF1 molecule has a sequence according to SEQ ID NO: 208, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto;
e) the ORF1 molecule has a sequence according to SEQ ID NO: 205, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto;
f) the ORF1 molecule has a sequence according to SEQ ID NO: 201, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto; or
g) the ORF1 molecule has a sequence according to SEQ ID NO: 1012, or a polypeptide comprising an amino acid sequence having at least about 70%, 75%, 80%, 85%, 90%, 95%, 96%, 97%, 98%, or 99% sequence identity thereto, and the Anellovirus GC-rich region has a sequence of the reverse complement of the Anellovirus GC-rich region of SEQ ID NO: 1, or a nucleic acid sequence having at least about 70%, 75%, 80%, 85%, 90%, 95%, 96%, 97%, 98%, or 99% sequence identity thereto.
691. An anellovector comprising:
(i) a proteinaceous exterior comprising an ORF1 molecule having a sequence according to SEQ ID NO: 58, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
692. An anellovector comprising:
(i) a proteinaceous exterior comprising an ORF1 molecule having a sequence according to SEQ ID NO: 1005, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
693. An anellovector comprising:
(i) a proteinaceous exterior comprising an ORF1 molecule having a sequence according to SEQ ID NO: 211, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
694. An anellovector comprising:
(i) a proteinaceous exterior comprising an ORF1 molecule having a sequence according to SEQ ID NO: 208, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
695. An anellovector comprising:
(i) a proteinaceous exterior comprising an ORF1 molecule having a sequence according to SEQ ID NO: 205, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
696. An anellovector comprising:
(i) a proteinaceous exterior comprising an ORF1 molecule having a sequence according to SEQ ID NO: 201, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
697. An anellovector comprising:
(i) a proteinaceous exterior comprising an ORF1 molecule having a sequence according to SEQ ID NO: 1012, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises:
a nucleic acid sequence encoding an exogenous effector; and
one or both of:
   (a) an Anellovirus GC-rich region, wherein the Anellovirus GC-rich region has a sequence of the reverse complement of the Anellovirus GC-rich region of SEQ ID NO: 1, or a nucleic acid sequence having at least about 90%, 95%, 96%, 97%, 98%, or 99% sequence identity thereto; and
   (b) an Anellovirus 5′ UTR conserved domain, wherein the Anellovirus 5′ UTR conserved domain has a sequence of the reverse complement of the Anellovirus 5′ UTR conserved domain of SEQ ID NO: 1, or a nucleic acid sequence having at least about 90%, 95%, 96%, 97%, 98%, or 99% sequence identity thereto.
698. The anellovector of any of the preceding embodiments, wherein the genetic element lacks an Anellovirus CG-rich region of the first Anellovirus genomic sequence.
699. The anellovector of any of the preceding embodiments, which lacks an ORF1 molecule of the second Anellovirus genomic sequence.
700. The anellovector of any of the preceding embodiments, wherein the genetic element comprises an Anellovirus 5′ UTR conserved domain, e.g., wherein the Anellovirus 5′ UTR conserved domain has a sequence of the reverse complement of the 5′ UTR Conserved Domain of any of Tables N1-N32, or a nucleic acid sequence having at least about 70%, 75%, 80%, 85%, 90%, 95%, 96%, 97%, 98%, or 99% sequence identity thereto.
701. The anellovector of any of the preceding embodiments, wherein the genetic element comprises an Anellovirus non-coding region (NCR), e.g., wherein the Anellovirus NCR has a sequence of the reverse complement of the Anellovirus NCR of any of Tables N1-N32, or a nucleic acid sequence having at least about 70%, 75%, 80%, 85%, 90%, 95%, 96%, 97%, 98%, or 99% sequence identity thereto.
702. The anellovector of any of the preceding embodiments, wherein the ORF1 molecule comprises an arginine-rich region that binds the Anellovirus GC-rich region.
703. The anellovector of any of the preceding embodiments, wherein the first Anellovirus genomic sequence is a wild-type Anellovirus genome sequence (e.g., as described herein, e.g., in any of Tables N1-N32).
704. The anellovector of any of the preceding embodiments, wherein the second Anellovirus genomic sequence is a wild-type Anellovirus genome sequence (e.g., as described herein, e.g., in any of Tables N1-N32).
705. The anellovector of any of the preceding embodiments, wherein the genetic element does not comprise an inverted terminal repeat (ITR) (e.g., an AAV ITR), or a nucleic acid sequence having at least 90%, 95%, 96%, 97%, 98%, 99%, or 100% identity thereto.
706. The anellovector of any of the preceding embodiments, wherein the genetic element does not comprise a contiguous nucleic acid sequence of at least 50, 60, 70, 80, 90, 100, 150, 200, 250, 300, 400, or 500 nucleotides in length having at least 90%, 95%, 96%, 97%, 98%, 99%, or 100% identity to a contiguous nucleic acid sequence from an AAV genome sequence.
707. The anellovector of any of the preceding embodiments, wherein the genetic element is DNA.
708. The anellovector of any of the preceding embodiments, wherein the genetic element is circular, single stranded DNA.
709. The anellovector of embodiment 707 or 708, wherein the genetic element comprises a promoter element operably linked to the nucleic acid sequence encoding the exogenous effector.
710. The anellovector of any of embodiments 662-706, wherein the genetic element is RNA, e.g., mRNA.
711. The anellovector of any of the preceding embodiments, wherein the exogenous effector comprises: an intracellular peptide, an intracellular polypeptide, a secreted polypeptide, or a protein replacement therapeutic.
712. An anellovector comprising:
(i) a proteinaceous exterior comprising an ORF1 molecule having an ORF1 sequence of SEQ ID NO: 211, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.
713. An anellovector comprising:
(i) a proteinaceous exterior comprising a polypeptide encoded by an Anellovirus ORF1 nucleic acid sequence as listed in Table N32, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.
714. An anellovector comprising:
(i) a proteinaceous exterior (e.g., comprising an Anellovirus ORF1 molecule, e.g., as described herein, e.g., of SEQ ID NO: 211, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises:
(a) one or both of:
   (a1) an Anellovirus GC-rich region, wherein the Anellovirus GC-rich region has a sequence of the reverse complement of the Anellovirus GC-rich region of SEQ ID NO: 210, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 210, or a nucleic acid sequence having at least about 90%, 95%, 96%, 97%, 98%, or 99% sequence identity thereto; and
(b) a promoter element operably linked to a nucleic acid sequence (e.g., a DNA sequence) encoding an exogenous effector.
715. An isolated nucleic acid molecule (e.g., a genetic element construct or a genetic element) comprising:
(a) one or both of:
   (a1) an Anellovirus GC-rich region, wherein the Anellovirus GC-rich region has a sequence of the reverse complement of the Anellovirus GC-rich region of SEQ ID NO: 210, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 210, or a nucleic acid sequence having at least about 90%, 95%, 96%, 97%, 98%, or 99% sequence identity thereto; and
(b) a promoter element operably linked to a nucleic acid sequence (e.g., a DNA sequence) encoding an exogenous effector.
716. An anellovector comprising:
(i) a proteinaceous exterior comprising an ORF1 molecule having an ORF1 sequence of SEQ ID NO: 208, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.
717. An anellovector comprising:
(i) a proteinaceous exterior comprising a polypeptide encoded by an Anellovirus ORF1 nucleic acid sequence as listed in Table N31, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.
718. An anellovector comprising:
(i) a proteinaceous exterior (e.g., comprising an Anellovirus ORF1 molecule, e.g., as described herein, e.g., of SEQ ID NO: 208, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises:
(a) one or both of:
   (a1) an Anellovirus GC-rich region, wherein the Anellovirus GC-rich region has a sequence of the reverse complement of the Anellovirus GC-rich region of SEQ ID NO: 207, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 207, or a nucleic acid sequence having at least about 90%, 95%, 96%, 97%, 98%, or 99% sequence identity thereto;
(b) a promoter element operably linked to a nucleic acid sequence (e.g., a DNA sequence) encoding an exogenous effector.
719. An isolated nucleic acid molecule (e.g., a genetic element construct or a genetic element) comprising:
(a) one or both of:
   (a1) an Anellovirus GC-rich region, wherein the Anellovirus GC-rich region has a sequence of the reverse complement of the Anellovirus GC-rich region of SEQ ID NO: 207, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 207, or a nucleic acid sequence having at least about 90%, 95%, 96%, 97%, 98%, or 99% sequence identity thereto; and
(b) a promoter element operably linked to a nucleic acid sequence (e.g., a DNA sequence) encoding an exogenous effector.
720. An anellovector comprising:
(i) a proteinaceous exterior comprising an ORF1 molecule having an ORF1 sequence of SEQ ID NO: 205, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence (e.g., a DNA sequence) encoding an exogenous effector.
721. An anellovector comprising:
(i) a proteinaceous exterior comprising a polypeptide encoded by an Anellovirus ORF1 nucleic acid sequence as listed in Table N30, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence, and
(ii) a genetic element enclosed by the proteinaceous exterior, wherein the genetic element comprises a promoter element operably linked to a nucleic acid sequence (e.g., a DNA sequence) encoding an exogenous effector.
722. An anellovector comprising:
(i) a proteinaceous exterior (e.g., comprising an Anellovirus ORF1 molecule, e.g., as described herein, e.g., of SEQ ID NO: 205, or a polypeptide comprising an amino acid sequence having at least about 70%, 75%, 80%, 85%, 90%, 95%, 96%, 97%, 98%, or 99% sequence identity thereto), and
(ii) a genetic element enclosed by the proteinaceous exterior, wherein the genetic element comprises:
(a) one or both of:
   (a1) an Anellovirus GC-rich region, wherein the Anellovirus GC-rich region has a sequence of the reverse complement of the Anellovirus GC-rich region of SEQ ID NO: 204, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 204, or a nucleic acid sequence having at least about 90%, 95%, 96%, 97%, 98%, or 99% sequence identity thereto;
(b) a promoter element operably linked to a nucleic acid sequence (e.g., a DNA sequence) encoding an exogenous effector.
723. An isolated nucleic acid molecule (e.g., a genetic element construct or a genetic element) comprising:
(a) one or both of:
   (a1) an Anellovirus GC-rich region, wherein the Anellovirus GC-rich region has a sequence of the reverse complement of the Anellovirus GC-rich region of SEQ ID NO: 204, or a nucleic acid sequence having at least about 90%, 95%, 96%, 97%, 98%, or 99% sequence identity thereto; and
   (a2) an Anellovirus 5′ UTR conserved domain, wherein the Anellovirus 5′ UTR conserved domain has a sequence of the reverse complement of the Anellovirus 5′ UTR conserved domain of SEQ ID NO: 204, or a nucleic acid sequence having at least about 90%, 95%, 96%, 97%, 98%, or 99% sequence identity thereto; and
(b) a promoter element operably linked to a nucleic acid sequence (e.g., a DNA sequence) encoding an exogenous effector.
724. An isolated cell comprising an anellovector of any of embodiments 662-714, 716-718, or 720-722.
725. An isolated cell comprising:
   (a) a nucleic acid molecule encoding the ORF1 molecule of any of SEQ ID NOs: 210, 207, or 204, or a nucleic acid sequence having at least about 90%, 95%, 96%, 97%, 98%, or 99% sequence identity thereto, or an Anellovirus ORF1 nucleic acid sequence as listed in any of Tables N30-N32, or a nucleic acid sequence having at least about 70%, 75%, 80%, 85%, 90%, 95%, 96%, 97%, 98%, or 99% sequence identity thereto; and
   (b) a genetic element construct or a genetic element (e.g., as described herein).
726. An isolated cell comprising:
   (a) an ORF1 molecule of any of SEQ ID NOs: 211, 208, or 205, or an amino acid sequence having at least about 90%, 95%, 96%, 97%, 98%, or 99% sequence identity thereto or a polypeptide encoded by an Anellovirus ORF1 nucleic acid sequence as listed in any of Tables N30-N32, or a polypeptide encoded by a nucleic acid sequence having at least about 70%, 75%, 80%, 85%, 90%, 95%, 96%, 97%, 98%, or 99% sequence identity to the Anellovirus ORF1 nucleic acid sequence; and
   (b) a genetic element construct (e.g., as described herein) or a genetic element.
727. A method of manufacturing an anellovector composition comprising the anellovector of any of embodiments 662-714, 716-718, or 720-722, the method comprising:
(a) providing a cell, e.g., a host cell as described herein;
(b) introducing a nucleic acid molecule encoding the ORF1 molecule;
(c) introducing into the cell a genetic element construct comprising the sequence of the genetic element (e.g., before, after, or simultaneously with (b)),
(d) incubating the cell under conditions that allow the cell to produce anellovector; and
(e) formulating the anellovector, e.g., as a pharmaceutical composition suitable for administration to a subject,
thereby making the anellovector composition.
728. A method of manufacturing an anellovector composition comprising the anellovector of any of embodiments 662-714, 716-718, or 720-722, the method comprising:
(a) providing an ORF1 molecule;
(b) contacting the ORF1 molecule with a genetic element in a cell-free system under conditions that allow for formation of the proteinaceous exterior comprising the ORF1 molecule, wherein the genetic element is enclosed by the proteinaceous exterior, and
(c) formulating the anellovector, e.g., as a pharmaceutical composition suitable for administration to a subject,
thereby making the anellovector composition.
729. A method of delivering an exogenous effector to a cell, tissue, or subject, the method comprising administering to the cell, tissue, or subject an anellovector of any of embodiments 662-714, 716-718, or 720-722.
730. The method of embodiment 729, wherein the tissue comprises an eye tissue, e.g., the retina or posterior eye cup.
731. The method of embodiment 729, wherein the tissue comprises lung, muscle, bone marrow, blood, heart, GI or skin.
732. The Anelloviridae family vector (e.g., anellovector) of any of the preceding embodiments, wherein the proteinaceous exterior (e.g., ORF1 molecule of the proteinaceous exterior) comprises the amino acid sequence YNPX²DXGX²N (SEQ ID NO: 829), wherein Xⁿ is a contiguous sequence of any n amino acids.
733. The Anelloviridae family vector of claim 732, wherein the amino acid sequence YNPX²DXGX²N (SEQ ID NO: 829) is comprised in an N22 domain of the ORF1 molecule.
734. The Anelloviridae family vector of any of the preceding embodiments, wherein the ORF1 molecule comprises one or more (e.g., 1, 2, 3, 4, or all 5) of the following Anellovirus ORF1 subdomains: an arginine-rich region, a jelly-roll region, a hypervariable region, an N22 domain, a C-terminal domain (CTD) (e.g., as described herein), e.g., of an Anellovirus ORF1 protein as listed in Table A1-A32 (or a sequence having at least about 70%, 75%, 80%, 85%, 90%, 95%, 96%, 97%, 98%, 99%, or 100% sequence identity thereto).
735. The isolated nucleic acid molecule of any of embodiments 715, 719, or 723, wherein the isolated nucleic acid molecule comprises at least one difference (e.g., a mutation, chemical modification, or epigenetic alteration) relative to a wild-type Anellovirus genome sequence (e.g., as described herein)
736. The isolated nucleic acid molecule of embodiment 735, wherein the at least one difference comprises a deletion (e.g., lacks one or more of: a 5′ UTR conserved domain, an ORF1 gene, ORF2 gene, a GC-rich region, an ORF3 gene, or a functional fragment thereof).
737. The isolated nucleic acid molecule of any of embodiments 715, 719, 723, 735 or 736, wherein the isolated nucleic acid molecule is substantially unable to be enclosed in an Anellovirus capsid (e.g., a proteinaceous exterior of an Anelloviridae family vector (e.g., anellovector) as described herein).
738. The isolated nucleic acid molecule of any of embodiments 715, 719, 723, or 735-737, wherein the isolated nucleic acid molecule encodes an effector (e.g., an exogenous effector or an endogenous effector).
739. The genetic element of any of the preceding embodiments, which further comprises a promoter.
740. The genetic element of any of the preceding embodiments, which further comprises a poly A sequence.
741. The pharmaceutical composition of any of the preceding embodiments, wherein the pharmaceutical composition has one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants;
e) the pharmaceutical composition has a predetermined level of non-infectious particles or a predetermined ratio of particles:infectious units (e.g., <300:1, <200:1, <100:1, or <50:1), or
f) the pharmaceutical composition has low immunogenicity or is substantially non-immunogenic, e.g., as described herein.
742. The pharmaceutical composition of any one of the preceding embodiments, wherein the pharmaceutical composition has a contaminant level below a predetermined reference value, e.g., is substantially free of contaminants.
743. The pharmaceutical composition of embodiment 742, wherein the contaminant is selected from the group consisting of: mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted Anelloviridae family vector (e.g., anellovector) (e.g., an Anelloviridae family vector other than the desired Anelloviridae family vector, e.g., a synthetic Anelloviridae family vector as described herein), free viral capsid protein, adventitious agents, and aggregates.
744. The pharmaceutical composition of embodiment 742, wherein the contaminant is host cell DNA and the threshold amount is about 10 ng of host cell DNA per dose of the pharmaceutical composition.
745. The pharmaceutical composition of any one of the preceding embodiments, wherein the pharmaceutical composition comprises less than 10% (e.g., less than about 10%, 5%, 4%, 3%, 2%, 1%, 0.5%, or 0.1%) contaminant by weight.
746. The genetic element, Anelloviridae family vector (e.g., anellovector), nucleic acid molecule, or method of any of the preceding embodiments, wherein the genetic element is single-stranded.
747. The genetic element, Anelloviridae family vector (e.g., anellovector), nucleic acid molecule, or method of any of the preceding embodiments, wherein the genetic element is circular.
748. The genetic element, Anelloviridae family vector (e.g., anellovector), nucleic acid molecule, or method of any of the preceding embodiments, wherein the genetic element comprises DNA.
749. The genetic element, Anelloviridae family vector (e.g., anellovector), nucleic acid molecule, or method of any of the preceding embodiments, wherein the genetic element is a negative strand DNA.
750. The genetic element, Anelloviridae family vector (e.g., anellovector), nucleic acid molecule, or method of any of the preceding embodiments, wherein the genetic element is double-stranded.
751. The genetic element, Anelloviridae family vector (e.g., anellovector), nucleic acid molecule, or method of any of the preceding embodiments, wherein the genetic element is linear.
752. The genetic element, Anelloviridae family vector (e.g., anellovector), nucleic acid molecule, or method of any of the preceding embodiments, wherein the genetic element comprises RNA.
753. The genetic element, Anelloviridae family vector (e.g., anellovector), nucleic acid molecule, or method of any of the preceding embodiments, wherein the genetic element comprises a nucleic acid sequence encoding an Anelloviridae capsid protein, e.g., an Anellovirus ORF1 molecule (e.g., an ORF1 protein as listed in Table A1-A32 or an amino acid sequence having at least 70%, 75%, 80%, 85%, 90%, 95%, 96%, 97%, 98%, or 99% sequence identity thereto).
754. The genetic element, Anelloviridae family vector (e.g., anellovector), nucleic acid molecule, or method of any of the preceding embodiments, wherein the genetic element does not comprise a nucleic acid sequence encoding an Anelloviridae capsid protein, e.g., an Anellovirus ORF1 molecule (e.g., an ORF1 protein as listed in Table A1-A32 or an amino acid sequence having at least 70%, 75%, 80%, 85%, 90%, 95%, 96%, 97%, 98%, or 99% sequence identity thereto).
755. The genetic element, Anelloviridae family vector (e.g., anellovector), nucleic acid molecule, or method of any of the preceding embodiments, wherein the genetic element comprises a nucleic acid sequence encoding an Anellovirus ORF2 molecule (e.g., an ORF2 protein as listed in Table A1-A32, or an amino acid sequence having at least 70%, 75%, 80%, 85%, 90%, 95%, 96%, 97%, 98%, or 99% sequence identity thereto).
756. The genetic element, Anelloviridae family vector (e.g., anellovector), nucleic acid molecule, or method of any of the preceding embodiments, wherein the genetic element does not comprise a nucleic acid sequence encoding an Anellovirus ORF2 molecule (e.g., an ORF2 protein as listed in Table A1-A32 or an amino acid sequence having at least 70%, 75%, 80%, 85%, 90%, 95%, 96%, 97%, 98%, or 99% sequence identity thereto).
757. The genetic element, Anelloviridae family vector (e.g., anellovector), nucleic acid molecule, or method of any of the preceding embodiments, wherein the genetic element comprises at least 20, 25, 30, 31, 32, 33, 34, 35, 36, 37, 38, 39, or 40 consecutive nucleotides having a GC content of at least 70%, 75%, 80%, 85%, 90%, 95%, or 99%.
758. The Anelloviridae family vector (e.g., anellovector), nucleic acid molecule, or method of any of the preceding embodiments, wherein the proteinaceous exterior comprises the amino acid sequence YNPX²DXGX²N (SEQ ID NO: 829), wherein Xⁿ is a contiguous sequence of any n amino acids.
759. The Anelloviridae family vector (e.g., anellovector), nucleic acid molecule, or method of embodiment 758, wherein the amino acid sequence YNPX²DXGX²N (SEQ ID NO: 829) is comprised in an N22 domain.
760. The Anelloviridae family vector (e.g., anellovector), nucleic acid molecule, or method of any of the preceding embodiments, wherein the ORF1 molecule comprises an arginine-rich region (e.g., having at least 70%, 80%, 85%, 90%, 95%, 96%, 97%, 98%, 99%, or 100% identity to an arginine-rich region sequence of an ORF1 protein listed in Table A1-A32).
761. The Anelloviridae family vector (e.g., anellovector), nucleic acid molecule, or method of any of the preceding embodiments, wherein the proteinaceous exterior comprises an amino acid sequence of at least 15, 20, 25, 26, 27, 28, 29, 30, 31, 32, 33, 34, 35, 36, 37, 38, 39, 40, 45, or 50 consecutive nucleotides comprising at least 40% (e.g., at least 40%, 41%, 42%, 43%, 44%, 45%, 46%, 47%, 48%, 49%, 50%, 55%, 60%, 65%, 66%, 67%, 68%, 69%, 70%, 75%, 80%, 85%, 90%, or 95%) arginine residues.
762. The Anelloviridae family vector (e.g., anellovector), nucleic acid molecule, or method of embodiment 760 or 761, wherein the arginine-rich region is located at the N-terminal or C-terminal end of the ORF1 molecule.
763. The genetic element, Anelloviridae family vector (e.g., anellovector), nucleic acid molecule, or method of any of the preceding embodiments, wherein the genetic element comprises one or more of: a TATA box, an initiator element, a cap site, a transcriptional start site, an ORF1/1-encoding sequence, an ORF1/2-encoding sequence, an ORF2/2-encoding sequence, an ORF2/3-encoding sequence, an ORF2/3 t-encoding sequence, a three open-reading frame region, a poly(A) signal, and/or a GC-rich region from an Anellovirus described herein, or a sequence having at least 70%, 75%, 80%, 85%, 90%, 95%, 96%, 97%, 98%, 99%, or 100% sequence identity thereto.
764. The genetic element, Anelloviridae family vector (e.g., anellovector), nucleic acid molecule, or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to a 5′ UTR conserved domain sequence as described herein.
765. The genetic element, Anelloviridae family vector (e.g., anellovector), nucleic acid molecule, or method of any of the preceding embodiments, wherein the genetic element comprises at least 75% (e.g., at least 75, 76, 77, 78, 79, 80, 85, 90, 91, 92, 93, 94, 95, 96, 97, 98, 99, or 100%) sequence identity to a GC-rich region sequence as described herein.
766. The genetic element, Anelloviridae family vector (e.g., anellovector), nucleic acid molecule, or method of any of the preceding embodiments, wherein the effector encodes a therapeutic agent, e.g., a therapeutic peptide or polypeptide or a therapeutic nucleic acid.
767. The genetic element, Anelloviridae family vector (e.g., anellovector), nucleic acid molecule, or method of any of the preceding embodiments, wherein the effector is an exogenous effector.
768. The Anelloviridae family vector (e.g., anellovector), nucleic acid molecule, or method of any of the preceding embodiments, wherein the effector is an endogenous effector (e.g., wherein the anellovector overexpresses the endogenous effector in a target cell).
769. The genetic element, Anelloviridae family vector (e.g., anellovector), nucleic acid molecule, or method of any of the preceding embodiments, wherein the effector modulates expression or activity of a gene or protein, e.g., increases or decreases expression or activity of the gene or protein.
770. The Anelloviridae family vector (e.g., anellovector), nucleic acid molecule, or method of any of the preceding embodiments, wherein the Anelloviridae family vector is capable of replicating autonomously.
771. The Anelloviridae family vector (e.g., anellovector), nucleic acid molecule, or method of any of the preceding embodiments, wherein the Anelloviridae family vector is replication-deficient (e.g., incapable of replicating autonomously).
772. The genetic element, Anelloviridae family vector (e.g., anellovector), nucleic acid molecule, or method of any of the preceding embodiments, wherein the genetic element integrates into the genome of a eukaryotic cell at a frequency of less than about 0.001%, 0.005%, 0.01%, 0.05%, 0.1%, 0.5%, 1%, 1.5%, or 2% of the genetic element that enters the cell.
773. The Anelloviridae family vector (e.g., anellovector), nucleic acid molecule, or method of any of the preceding embodiments, wherein the Anelloviridae family vector is substantially non-pathogenic, e.g., does not induce a detectable deleterious symptom in a subject (e.g., elevated cell death or toxicity, e.g., relative to a subject not exposed to the anellovector).
774. The Anelloviridae family vector (e.g., anellovector), nucleic acid molecule, or method of any of the preceding embodiments, wherein the Anelloviridae family vector is substantially non-immunogenic, e.g., does not induce a detectable and/or unwanted immune response.
775. The Anelloviridae family vector (e.g., anellovector), nucleic acid molecule, or method of any of the preceding embodiments, wherein a population of at least 1000 of the Anelloviridae family vectors is capable of delivering at least about 100 copies (e.g., at least 1, 2, 3, 4, 5, 10, 20, 30, 40, 50, 100, 200, 300, 400, 500, 600, 700, 800, 900, or 1000 copies) of the genetic element into one or more eukaryotic cells (e.g., mammalian cells, e.g., human cells).
776. The Anelloviridae family vector (e.g., anellovector), nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering the genetic element into at least 10%, 20%, 30%, 40%, 50%, 60%, 70%, 80%, 90%, 95%, 99%, or more of a population of eukaryotic cells (e.g., mammalian cells, e.g., human cells).
777. The Anelloviridae family vector (e.g., anellovector), nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering at least 1, 2, 3, 4, 5, 6, 7, 8, 9, 10, 20, 50, 100, 200, 500, 1000, 2000, 5000, 8,000, 1×10⁴, 1×10⁵, 1×10⁶, 1×10⁷ or greater copies of the genetic element per cell to a population of eukaryotic cells (e.g., mammalian cells, e.g., human cells).
778. The Anelloviridae family vector (e.g., anellovector), nucleic acid molecule, or method of any of the preceding embodiments, wherein a population of the Anelloviridae family vectors (e.g., at least 1, 2, 3, 4, 5, 10, 20, 30, 40, 50, 100, 200, 300, 400, 500, 600, 700, 800, 900, or 1000 genome equivalents of the genetic element per cell) is capable of delivering 1-3, 1-4, 1-5, 1-6, 1-7, 1-8, 1-9, 1-10, 5-10, 10-20, 20-50, 50-100, 100-1000, 1000-10⁴, 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of the genetic element per cell to a population of eukaryotic cells (e.g., mammalian cells, e.g., human cells).
779. The Anelloviridae family vector (e.g., anellovector), nucleic acid molecule, or method of any of the preceding embodiments, wherein the target cells into which the genetic element is delivered each receive at least 10, 50, 100, 500, 1000, 10,000, 50,000, 100,000, or more copies of the genetic element.
780. The Anelloviridae family vector (e.g., anellovector), nucleic acid molecule, or method of any of the preceding embodiments, wherein the Anelloviridae family vector is resistant to degradation by a detergent (e.g., a mild detergent, e.g., a biliary salt, e.g., sodium deoxycholate) relative to a viral particle comprising an external lipid bilayer, e.g., a retrovirus.
781. The Anelloviridae family vector (e.g., anellovector), nucleic acid molecule, or method of any of the preceding embodiments, wherein the genetic element enclosed by the proteinaceous exterior is resistant to degradation by a nuclease enzyme (e.g., a DNase).
782. The Anelloviridae family vector (e.g., anellovector), nucleic acid molecule, or method of any of the preceding embodiments, wherein the Anelloviridae family vector is capable of infecting mammalian cells, e.g., human cells, e.g., in vitro, in vivo, or ex vivo.
783. The Anelloviridae family vector (e.g., anellovector), nucleic acid molecule, or method of any of the preceding embodiments, wherein the Anelloviridae family vector selectively delivers the effector to, or is present at higher levels in (e.g., preferentially accumulates in), a desired cell type, tissue, or organ.
784. The Anelloviridae family vector (e.g., anellovector), nucleic acid molecule, or method of any of the preceding embodiments, wherein the proteinaceous exterior is provided in cis relative to the genetic element.
785. The Anelloviridae family vector (e.g., anellovector), nucleic acid molecule, or method of any of the preceding embodiments, wherein the proteinaceous exterior is provided in trans relative to the genetic element.
786. The method of any of embodiments 727-731, wherein the Anelloviridae family vector is substantially free of wild-type Anellovirus genomes.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings. The patent or application file contains at least one drawing executed in color. Copies of this patent or patent application publication with color drawing(s) will be provided by the Office upon request and payment of the necessary fee.

FIG. 1 is a series of diagrams showing production of exemplary malaria peptide conjugation constructs comprising the C-terminal region of a CS protein.

FIG. 2 is a series of diagrams showing the structure of an exemplary malaria peptide conjugation construct on the surface of an anellovector capsid.

FIG. 3 is a diagram showing separation of in vitro circularized DNA into positive and negative sense circularized single-stranded DNA (ssDNA) after denaturation.

FIGS. 4A and 4B are a series of diagrams showing successful expression of Anellovirus ORF1 molecules in cells. (A) Anellovirus strains for which ORF1 expression was detected from Sf9 cells included Ring2, Ring3, Ring4, Ring5, Ring6, Ring9, and Ring10 (e.g., as described herein). (B) Virus-like particles (VLPs) were observed by electron microscopy after production of Anellovirus ORF1 proteins for Ring2 and Ring10, as well as for chicken anemia virus (CAV) VP1.

FIGS. 5A-5C are a series of diagrams showing successful expression and purification of Ring2 ORF1 and ORF2 proteins from Sf9 cells. (A) Exemplary workflow for purification of ORF1 and ORF2 proteins from Sf9 cells. (B) Western blot using anti-ORF1 antibody, showing detection of Ring2 ORF1 proteins produced in Sf9 cells as described. (C) Electron micrograph showing formation of virus-like particles (VLPs) from Ring2 ORF1 proteins produced in Sf9 cells as described.

FIGS. 6A-6C are a series of diagrams showing disassembly of Ring2 VLPs using urea as a chaotropic denaturant. (A) Ring2 ORF1 protein was produced and purified from Sf9 cells as described, and VLPs were detected by electron microscopy. (B) After treatment with urea, VLPs are no longer observed, but instead the solution contains small ORF1 capsomers. (C) Expanded view of the boxed area shown in FIG. 6B, showing detail of the capsomers.

FIGS. 7A-7B are a series of electron micrographs showing exemplary symmetrical (A) and asymmetrical (B) particle morphologies.

FIG. 8 is a schematic showing an exemplary workflow for production of anellovectors (e.g., replication-competent or replication-deficient anellovectors as described herein).

FIG. 9 is a diagram showing an alignment of 36-nucleotide GC-rich regions from nine Anellovirus genome sequences, and a consensus sequence based thereon (SEQ ID NOS 818-827, respectively, in order of appearance).

FIG. 10 is a series of diagrams showing ORF1 structures from Anellovirus strains LY2 and CBD203. Putative domains are labeled: arginine-rich region (arg-rich), core region comprising a structural jelly-roll domain, hypervariable region (HVR), structural N22 region, and structural C-terminal domain (CTD), as indicated.

FIG. 11A is a schematic presentation of full-length Ring 10 ORF1 (also referred to herein as Ly1) labeled and colored by domains. The structural arginine-rich motif (ARM) is shown in purple, the structural jelly roll (JR) domain is shown in red, the spike P1 domain is shown in blue, the spike P2 domain is shown in green, and the C-terminal domain is shown in cyan. Residue numbers beginning each domain and the structural C-terminal domain (also referred to as the C-terminus) are indicated above.

FIG. 11B is the sequence of full-length Ring 10 ORF1 colored as in FIG. 14A with residue numbers indicated above. In bold are residues included in the Ring 10 delARM construct including the initial residue, K46, which is labeled. A dashed line above the sequence indicates residues not observed in the density. Secondary structure elements are indicated above with β-strands as arrows and α-helices as zig-zag lines. The JR β-strands are labeled B—I per convention while additional secondary structures are numbered by their domain. Three peptides used to generate polyclonal antibodies are underlined. Figure discloses SEQ ID NO: 2758.

FIG. 11C shows western blot analysis of Ring 10 delARM after expression (Expression) and after purification and storage (Purification). A molecular weight marker is labeled to the left of the gels, while arrows on the right indicate the band of Ring 10 delARM before (Ring 10 delARM) and after proteolysis (Ring 10 delARM Fragment). Polyclonal antibodies used to probe the western blots are indicated below and colored by the peptides used to generate them.

FIG. 11D is an overlay of the 3D reconstruction of Ring 10 delARM VLP electron density and 60-mer VLP molecular structure colored as in FIG. 14A. The spike P1 and P2 domains are labeled.

FIG. 11E depicts one ORF1 protomer, shown in its electron density with domains labeled and colored as in FIG. 14A.

FIG. 11F depicts the electron density of Ring 10 delARM VLP colored by its local resolution. The bar (left) indicates the resolution (unit in angstrom) scale by color. The particle (right) is oriented as in FIG. 14D.

FIG. 12A depicts 60 Ring 10 structural jelly roll (JR) domains with one uniquely colored in red. Sixty Ring 10 structural jelly roll (JR) domains form the core of anellovirus particles.

FIG. 12B depicts two JR domains, shown in red, with the observed C-terminal domain backbone colored in cyan. The JR domains are arbitrarily labeled JR¹ and JR² with the first (K48) and last (V562) observed residues for each protomer labeled with the corresponding number for clarity.

FIG. 12C depicts a single JR domain oriented to show the R-sheet on the interior of the particle core. Sidechains of basic residues in position to contact with the viral genome are shown and labeled.

FIG. 12D depicts the structural arginine-rich region, JR, and structural C-terminal domains of Ring 10 aligned with corresponding ORF1 sequences from different anellovirus genera (indicated in parentheses). Residues of Ring 10 are colored as in FIG. 14A-14D. Basic residues of Ring 10 positioned to potentially contact the viral genome are indicated with asterisks. Figure discloses SEQ ID NOS 2759-2762, respectively, in order of appearance.

FIG. 13A depicts the anellovirus particle structure as shown as a surface rendering. The particle is shown in gray with 5 spikes forming a crown structure, numbered for clarity and colored as in FIG. 11A-11D. The spike domains extend from the core on the 5-fold axis.

FIG. 13B depicts the exterior of the crown structure as shown from the side. Five spike domains are colored as in FIG. 16A. The hydrophobic and hydrophilic conserved residues are colored in light blue and magenta, respectively.

FIG. 13C depicts the same spike domain from FIG. 16B rotated to view residues on the interior of the crown structure.

FIG. 13D depicts the spike domain of Ring 10 (colored as in FIG. 1A-1D) aligned with ORF1 sequences representative of different anellovirus genera (indicated in parentheses). Magenta and black asterisks indicate the surface-exposed residues of the P1 domain and P2 domain, respectively. Below the alignment are >30% consensus residues, or Ø or γ indicating the residues are >70% hydrophobic or >60% hydrophilic, respectively. Figure discloses SEQ ID NOS 2763-2766, respectively, in order of appearance.

FIG. 14A is a schematic representation of full-length Ring 10 (top), which shows highly heterogeneous particles by negative-stained electron microscopy (bottom). Scale bar=100 nm.

FIG. 14B is a schematic representation of Ring 10 delARM (arginine-rich motif; top), which demonstrates a structural homology virus-like particle (VLP) as shown by negative-stained electron microscopy (bottom). Scale bar=100 nm.

FIG. 14C is a schematic representation of Ring 10 delARM delCTD (top), wherein further truncation of the structural C-terminal domain (Δ552-672) preserves a structured VLP as shown by negative-stained electron microscopy (bottom). Scale bar=100 nm.

FIG. 15 depicts a data processing procedure of the Ring 10 delARM cryogenic electron microscopy (cryo-EM) reconstruction. In short, crYOLO picked 58,391 particles from 11,083 micrographs. Several rounds of 2D classification resulted in 11,185 particles. After Relion de novo initial model reconstruction, Relion 3D refinement was implemented to obtain the orientation parameters. All particles with parameters were fed in a 3D classification. The class with the most abundant particle population resulted in 3.98 Å resolution.

FIG. 16A is a representative negative-stained micrograph of Ring 10 delARM. The micrograph was imaged at NanoImaging Service.

FIG. 16B is a representative cryo-EM micrograph of Ring 10 delARM. The micrograph was imaged at NanoImaging Service.

FIGS. 17A and 17B depict circular dichroism (CD) results of the TTMV-Ring 10 C-terminal peptide (CSEEEEESNLFERLLRQRTKQLQLKRRIIQTLKDLQKLE (SEQ ID NO: 2505)). FIG. 17A is a table showing averages of secondary structure fractions estimated by different packages of CDPro. α-helix dominates the secondary structure assignment from the CD spectrum. FIG. 17B shows an experimental spectrum of the C-terminal peptide (shown in red) overlaid with the calculated and averaged reference set spectra (shown in blue) from three different packages (SELCON3, CDSSTR, and CONTINLL).

FIG. 18 depicts sequence alignment of 15 known anelloviruses within different genera indicated in parentheses. The conserved amino acids are shown in the first blue row underneath the sequence. The top blue row underneath the sequence alignment indicates the homology sequence if the conservation is larger than 30%. The bottom blue row shows either hydrophobic (0, within 70% similarity) or 60% positive charged (γ, within 60% similarity) of amino acids, respectively. The alignment was done by Geneious-implemented Clustal Omega. Figure discloses SEQ ID NOS 1012, 58, 2746-2748, 1020, 2749, 2750, 2752, 2751, 2753, 2757, 2755, 2754, and 2756, respectively, in order of appearance.

FIG. 19A-19D depict the spikes of Ring 10 or alpha-fold predicted JA20 and MN779270.1. FIG. 19A depicts the sphere representation of Ring 10 spike (identical to FIG. 16C). FIGS. 19B and 19C are the spike representations for alpha-fold predicted JA20 and MN779270.1, respectively. P1 and P2 domains are demonstrated in blue and green. The light blue and magenta are the conserved hydrophobic and basic residues. FIG. 19D shows the sequence alignment between Ring 10, JA20, and MN779210.1. Figure discloses SEQ ID NOS 2767-2770 respectively, in order of appearance.

FIG. 20 is a series of graphs showing that a polyclonal antibody (i.e., AB3725) that recognizes the surface-exposed Ring 10 HVR helix consisting of amino acid residues 352-361 of Ring 10 ORF1 specifically binds to Ring 10 anelloVLPs, but polyclonal antibodies generated against the HVR of different strains (shown as Strain 1 and Strain 2) did not bind to the Ring10 ORF1.

FIGS. 21A-21B show that AB3725 recognized Ring2 ORF1 mutants into which point mutations K357E, N358Q and E359I from Ring10 ORF1 were introduced. FIG. 21B discloses SEQ ID NOS 2778-2783, respectively, in order of appearance.

FIGS. 22A-22B are a series of diagrams showing a Coomassie stain and a Western blot for Ring2 virus-like particles (VLPs).

FIG. 23 is an electron micrograph showing Ring2 VLPs obtained after Capto400 purification.

FIGS. 24A-24B are a series of diagrams showing a Coomassie stain and a Western blot for Ring19 VLPs.

FIG. 25 is an electron micrograph showing Ring19 VLPs obtained after Capto400 purification.

FIG. 26 is a diagram showing an exemplary workflow for conjugating NHS ester moieties to surface lysines of anelloVLPs using click chemistry.

FIGS. 27A-27B are a series of diagrams showing a Coomassie stain and a Western blot for Ring2 VLPs conjugated with NHS Ester 647.

FIGS. 28A-28B are a series of diagrams showing Western blots for Ring2 anelloVLPs conjugated with NHS Ester biotin, labeled using a streptavidin CV 800 antibody or a Ring19 HVR3 primary antibody and a goat anti-rabbit secondary antibody.

FIG. 29 is a diagram showing an exemplary two-step process for conjugating a surface effector moiety to the surface of an anelloVLP.

FIG. 30 is a diagram showing Coomassie staining, Western blot, and UV labeling for SARS-CoV-2 receptor binding domains (RBD) attached to DBCO, CalFluor 488, Azide, or Alexa488, as shown.

FIG. 31 is a diagram showing Coomassie staining and Western blot for pRTx-2652 (a Ring 2 ORF1 variant) produced using SE-FPLC.

FIG. 32 is a diagram showing Coomassie staining and Western blot showing conjugation of RBD attached to an azide moiety to pRTx-2652 ORF1 polypeptide attached to DBCO, to produce RBD-pRTx-2652 conjugate anelloVLPs.

FIG. 33 is a schematic of an exemplary Cre-loxp based vector system. In this exemplary system, the genetic element sequence comprises, in 5′ to 3′ order, a 3′ NCR comprising a GC-rich region, a 5′ NCR, and a transgene sequence. The genetic element sequence is flanked by lox71 and lox66 sites. Introduction of a Cre recombinase results in excision and circularization of the loxP-flanked sequence to form a double-stranded minicircle comprising the genetic element sequence (shown in the figure as “Vector”). After conversion of the minicircle to a single-stranded circular DNA, Anelloviral proteins, provided in trans, then form a proteinaceous exterior comprising ORF1 molecules around the single-stranded circular DNA, thereby producing a packaged Anellovector.

FIG. 34 depicts the results of a post-iodixanol DNase-protection assay for batch 1 Anellovector Ring19-fCMV-eGFP material.

FIG. 35 depicts the results of a pre- and post-concentration DNase-protection assay for batch 1 Anellovector Ring19-fCMV-eGFP material, showing absence of Ring19 WT viral genomes.

FIG. 36 depicts coomassie (left panel) and silver stain (right panel) on batch 1 Anellovector Ring19-fCMV-eGFP material.

FIG. 37 depicts four plots of qPCR assays for Ring19-eGFP genetic material in the posterior eye cup (PEC) (top of first page) and the retina (bottom of first page), and WT Ring19 genetic material in the PEC (top of second page) and retina (bottom of second page), showing the presence of eGFP DNA in the retina 21 days after viral transduction.

FIG. 38 depicts the results of a post-iodixanol DNase-protection assay for batch 2 Anellovector Ring19-fCMV-eGFP material.

FIG. 39 depicts the results of a pre- and post-concentration DNase-protection assay for batch 2 Anellovector Ring19-fCMV-eGFP material, showing absence of Ring19 WT viral genomes.

FIG. 40 depicts coomassie staining on batch 2 Anellovector Ring19-fCMV-eGFP material.

FIG. 41 depicts qPCR data in plots showing the presence of Ring19-eGFP DNA in the PEC 21 (top panel) and 49 (bottom panel) days after viral transduction.

FIG. 42 depicts qPCR data in plots showing Ring19-eGFP DNA in the retina 21 (top panel) and 49 (bottom panel) days after viral transduction, showing persistence of viral infection.

FIG. 43 depicts qPCR data for WT Ring19 DNA in the PEC 21 (top panel) and 49 (bottom panel) days after viral transduction, showing the lack of WT Ring19 DNA.

FIG. 44 depicts qPCR data for WT Ring19 DNA in the retina 21 (top panel) and 49 (bottom panel) days after viral transduction, showing the lack of WT Ring19 DNA.

FIG. 45 depicts plots showing RNA copies of eGFP detected by RT-qPCR in the PEC (top panel) and the retina (bottom panel), showing successful eGFP transduction by Ring19-eGFP in experiment 1.

FIG. 46 depicts plots showing RNA copies of eGFP detected by RT-ddPCR in the PEC (top panel) and the retina (bottom panel), showing successful transduction by Ring19-eGFP in experiment 1.

FIG. 47 depicts results from experiment 2 showing eGFP mRNA expression in the PEC by RT-qPCR (top panel first page) and RT-ddPCR (bottom panel first page) and eGFP mRNA expression in the retina by RT-qPCR (top panel second page) and RT-ddPCR (bottom panel second page) 21 days after viral transduction. These data indicate successful infection of eye tissue by Ring19-eGFP.

FIG. 48 depicts RT-qPCR (top panel) and RT-ddPCR (bottom panel) data showing eGFP mRNA expression in the PEC 49 days after viral transduction, showing infection by Ring19-eGFP lasts at least 49 days after transduction.

FIG. 49 depicts RT-qPCR (top panel) and RT-ddPCR (bottom panel) data showing eGFP mRNA expression in the retina 49 days after viral transduction.

FIG. 50 depicts fluorescent imaging of flatmount preparations of mouse PEC. The top row shows GFP expression (A), red blood cell autofluorescence in the Texas red channel (B), and a merge (C) for PBS treated negative control cells. The second row shows GFP expression (D), red blood cell autofluorescence in the Texas red channel (E), and a merge (F) for Ring19-eGFP infected cells. The third row shows GFP expression (G), red blood cell autofluorescence in the Texas red channel (H), and a merge (I) for dose matched AAV2-eGFP infected cells. The bottom row shows GFP expression (J), red blood cell autofluorescence in the Texas red channel (K), and a merge (L) for high dose AAV2-eGFP infected cells. Arrows indicate GFP-expressing cells.

FIG. 51 is a transmission electron microscopy (TEM) image of Ring19-eGFP virus showing successful virus assembly.

FIG. 52 depicts a retinal pigmented epithelial (RPE) cell culture at day 5 showing cell growth and confluence.

FIG. 53 depicts an RPE cell culture and cells spun down at day 28 showing visible melanin granule formation in the cells.

FIG. 54 depicts an RPE cell culture showing nuclei and ZO-1 staining around the cell membrane, showing tight junction formation in cultured cells.

FIG. 55 depicts a schematic of an ELISA assay against VEGF (left panel) and a bar graph that shows VEGF protein expression in RPE cell culture (right panel).

FIG. 56 shows human RPE cells transduced with Ring19-eGFP with phase imaging of cells (top panel), a single GFP positive cell in the middle of the field of view (middle panel), and merged phase and GFP images (bottompanel).

FIG. 57 shows human RPE cells transduced with AAV2-eGFP with phase imaging of cells (top panel), several GFP positive cells (middle panel), and merged phase and GFP images (bottom panel).

FIG. 58 depicts fluorescence microscopy images of RPE cells transduced by Ring19-eGFP showing GFP expression (top left panel), immunostaining for GFP (top right panel), Hoechst DNA staining (bottom left panel), and a merged image of all three channels (bottom right panel) at 20× magnification. GFP expression overlaps with staining for GFP in a cell in the bottom left of the image and a cell in the top left of the image, indicating successful transduction of eGFP.

FIG. 59 depicts fluorescence microscopy for Hoechst, a DNA stain (top left panel), GFP expression (top right panel), GFP immunostaining (bottom left panel), and all three channels merged (bottom right panel) in Ring19eGFP infected RPE cells at 40× magnification.

FIG. 60 is fluorescence microscopy images of RPE cells transduced by AAV2-eGFP showing GFP expression (top left panel), immunostaining for GFP (top right panel), Hoechst DNA staining (bottom left panel), and a merged image of all three channels (bottom right panel) at 20× magnification. GFP expression overlaps with staining for GFP in several cells, indicating successful transduction of eGFP.

FIG. 61 depicts fluorescence microscopy for Hoechst, a DNA stain (top left panel), GFP expression (top right panel), GFP immunostaining (bottom left panel), and all three channels merged (bottom right panel) in dose matched AAV2-eGFP infected RPE cells at 40× magnification.

FIG. 62 is fluorescence microscopy images of RPE cells not treated with virus showing GFP expression (top left), immunostaining for GFP (top right), Hoechst DNA staining (bottom left), and a merged image of all three channels (bottom right). There is no cell with GFP expression overlapping with staining for GFP, as expected for this negative control.

FIGS. 63A-63D depict four plots of qPCR assays for eGFP DNA in the posterior eye cup (PEC) (A) and the retina (B), and WT Ring19 DNA in the PEC (C) and retina (D), 21 days after viral transduction with R19-eGFP (LD), R19-eGFP (HD), AAV2-eGFP (LD), and AAV2-eGFP (HD), and a PBS negative control. Each dot represents one eye. N=3/group.

FIGS. 64A-64D depict plots showing eGFP mRNA expression in the PEC by RT-qPCR (A) and RT-ddPCR (B) and eGFP mRNA expression in the retina by RT-qPCR (C) and RT-ddPCR (D) 21 days after viral transduction with R19-eGFP (LD), R19-eGFP (HD), AAV2-eGFP (LD), and AAV2-eGFP (HD), and a PBS negative control. The two highest eGFP-expressing samples as determined by RT-qPCR were rerun with RT-ddPCR to confirm eGFP expression. N=5 for RT-qPCR and n=5 for RT-ddPCR.

FIG. 65 depicts fluorescent imaging of flatmount preparations of mouse PEC. The top row shows GFP expression (A), red blood cell autofluorescence in the Texas red channel (B), and a merge (C) for PBS treated negative control cells. The second row shows GFP expression (D), red blood cell autofluorescence in the Texas red channel (E), and a merge (F) for low dose (LD) Ring19-eGFP infected cells. The third row shows GFP expression (G), red blood cell autofluorescence in the Texas red channel (H), and a merge (I) for high dose (HD) Ring19-eGFP infected cells. The fourth row shows GFP expression (J), red blood cell autofluorescence in the Texas red channel (K), and a merge (L) for low dose (LD) AAV2-eGFP infected cells. The bottom row shows GFP expression (M), red blood cell autofluorescence in the Texas red channel (N), and a merge (O) for high dose (HD) AAV2-eGFP infected cells. Arrows indicate GFP-expressing cells. N=3/group.

FIG. 66 is an illustration of an exemplary recombinase based system for Anelloviridae family vector genetic element production. A minimum of two plasmids are used in the system. A first plasmid contains a promoter that drives expression of a site-specific recombinase. A second plasmid contains a transgene cassette comprising non-coding regions of an Anelloviridae family viral genome, promoter transgene, and terminator elements. The transgene cassette is flanked by recombinase recognition sites. The recombinase expressed from the first plasmid then recognizes the recognition sequences at either end of the cassette and initiates a recombination event, resulting in the production of a circular genetic element construct comprising the nucleic acid sequence of the genetic element of the desired Anelloviridae family vector.

FIG. 67 is an illustration of an exemplary Cre-based three-plasmid system that can be used to produce packaged Anelloviridae family vector particles. A first plasmid contains recombinase recognition sites (e.g. loxP sites), flanking an expression cassette with a non-coding region (or a portion thereof) of an Anelloviridae family virus, promoter, transgene, and terminator element. A second plasmid is an expression plasmid that provides the recombinase (e.g. Cre). The two plasmids together can result in production of a circular genetic element construct containing the sequence of the genetic element of the desired Anelloviridae family vector through expression of a Cre recombinase that initiates a recombination event in the first plasmid. A third plasmid is a self-replicating rescue (SRR) plasmid containing genes encoding Anelloviridae family viral proteins that drive, e.g., replication and packaging of the circular genomic elements, e.g. ORF1, ORF2, and/or ORF3. In some embodiments, the SRR plasmid also contains an SV40 origin of replication and optionally further expresses an SV40 large T antigen (eg. from an IRES sequence), allowing the SRR plasmid to be self-replicating and express the Anelloviridae family viral proteins at high levels. In some instances, a cell containing all three-plasmids produces packaged Anelloviridae family vector particles containing the desired vector sequence.

FIG. 68 depicts qPCR data in a bar plot comparing the amount of Ring19-eGFP genomes to wild type (WT) Ring19 genomes, showing an increase in Ring19-eGFP genome copies in the exemplary three-plasmid system compared to transfection of only a vector plasmid.

FIG. 69 is a bar graph showing the fold change in Ring19-eGFP genomes when transfecting different ratios of the three plasmids (as indicated) compared to an initial plasmid ratio of 50 ug:50 ug:50 ug.

FIG. 70 is an illustration of an exemplary Bxb1-based three-plasmid system that can be used to produce packaged Anelloviridae family vector particles. A first plasmid contains recombinase recognition sites, e.g. attP and attB, flanking an expression cassette with a non-coding region (or a portion thereof) of an Anelloviridae family virus, promoter, transgene, and terminator element. A second plasmid is an expression plasmid that provides the recombinase, e.g. Bxb1. The two plasmids together can result in production of a circular genetic element construct containing the nucleic acid sequence of the genetic element of a desired Anelloviridae family vector through expression of a Bxb1 recombinase that initiates a recombination even in the first plasmid. A third plasmid is a self-replicating rescue (SRR) plasmid encoding Anelloviridae family viral proteins that drive, e.g., replication and packaging of the circular genomic elements, e.g., ORF1, ORF2, and ORF3. In some embodiments, the SRR plasmid also contains an SV40 origin of replication and optionally further expresses an SV40 large T antigen (eg. from an IRES sequence), allowing the SRR plasmid to be self-replicating and express the Anelloviridae family viral proteins at high levels. A cell containing all three plasmids can, in some instances, produce packaged Anelloviridae family vector particles containing the desired vector sequence.

FIG. 71 depicts fluorescence microscopy images of MOLT-4 cells transfected with a plasmid with a CMV promoter-eGFP transgene expression cassette flanked by loxP sites (pFlox) (top left), a plasmid with a UbC promoter-eGFP transgene expression cassette flanked by attP and attB sites (pBox) (top center), the same pFlox plasmid in combination with a Ring19 SRR plasmid (top right), the same pBox plasmid with a Ring19 SRR (bottom left), mutated pBox2 plasmid with a Ring19 SRR (bottom center), and mutated pBox3 plasmid with a Ring19 SRR (bottom right).

FIG. 72 is a bar graph showing log-scale qPCR data for Ring19-eGFP and WT Ring19 genome copies produced from cells transfected with the pFlox vector with CMV as the eGFP transgene promoter or the pBox vectors (pBoxl, pBox2, and pBox3) with UbC as the eGFP transgene promoter, alone or together with a Ring19 self-replicating rescue plasmid (SRR) and a Cre or Bxb1-expressing plasmid.

FIGS. 73A-73B is a comparison of different promoters driving the eGFP payload. FIG. B16A illustrates the eGFP::WPRE::bGH-pA transgene remaining constant while the promoters (CMV, sCAG, CBA, and CBh) were swapped in each of the pBox and pFlox-based plasmids. FIG. B16B is a bar graph depicting the results of qPCR for Ring19-eGFP genetic material produced in vector genomes (vgs) per liter using the different promoters. As a negative control, MOLT-4 cells were transfected with a CMV-eGFP vector alone. For test samples, MOLT-4 cells were transfected with a CMV-eGFP vector and a Ring19 SRR, an sCAG-eGFP vector and a Ring19 SRR, a CBA-eGFP vector and a Ring19 SRR, or a CBh-eGFP vector and a Ring19 SRR. For pFlox-based plasmids, a plasmid encoding Cre was also transfected (pRTx-2848) and for pBox-based plasmids, a plasmid encoding Bxb1 was also transfected (pRTx-3542).

FIG. 74 is an exemplary schematic showing the addition of a separate origin of replication to the vector transgene cassette of an exemplary vector construct (top). In some embodiments, the origin of replication (depicted as a blue box) can be derived from AAV, PCV, SV40, or other viral origins of replication. Also shown is an exemplary self-replicating rescue (SRR) plasmid including promoter driven Anelloviridae family viral ORF coding sequences, SV40 Large T antigen, and SV40 origin of replication (middle), which can be used to provide viral proteins as described herein. The SRR plasmid can, in some instances, be introduced alongside a third plasmid comprising an expression cassette encoding replication initiator proteins for the selected origin of replication, e.g., to drive replication of the transgene plasmid. Together with expression of a recombinase, this system can be used to produce packaged Anelloviridae family vector particles containing the desired transgene cassette (right).

FIG. 75 is a schematic showing an exemplary two plasmid system for Anelloviridae family vector particle production. In this version, a first plasmid contains recombinase recognition sites, e.g. loxP sites, flanking an expression cassette comprising a non-coding region of an Anelloviridae family virus (or a portion thereof) followed by a promoter, transgene, and terminator element. This first plasmid also contains an expression cassette encoding the recombinase itself, e.g. Cre. Transfection of this plasmid can result in expression of the recombinase from the plasmid, which can then recombine the plasmid (or copies thereof) to produce a circular genetic element construct comprising the sequence of the genetic element of the desired Anelloviridae family vector. A second plasmid is a self-replicating rescue (SRR) plasmid encoding Anelloviridae family viral proteins that drive, e.g., replication and packaging of the circular genomic elements, e.g. ORF1, ORF2, and ORF3. In some embodiments, the SRR plasmid also contains an SV40 origin of replication and optionally further expresses an SV40 large T antigen (eg. from an IRES sequence), allowing the SRR plasmid to be self-replicating and express the Anelloviridae family viral proteins at high levels. A cell containing the first and second plasmids can thus produces packaged Anelloviridae family vector particles.

FIG. 76 is a schematic showing an exemplary two-plasmid system for production of Anelloviridae family vector particles using Bxb1 recombinase. In this version, a first plasmid contains recombinase recognition sites, e.g. attP and attB, flanking an expression cassette comprising a non-coding region of an Anelloviridae family virus (or a portion thereof), followed by a promoter, transgene, and terminator element. This first plasmid also contains an expression cassette encoding the corresponding recombinase, e.g., Bxb1. Transfection of this plasmid can result in expression of the recombinase from the plasmid, which can then recombine the plasmid (or copies thereof) to produce a circular genetic element comprising the sequence of the genetic element of the desired Anelloviridae family vector. A second plasmid is a self-replicating rescue (SRR) plasmid encoding Anelloviridae family viral proteins that drive, e.g., replication and packaging of the circular genomic elements, e.g. ORF1, ORF2, and ORF3. In some embodiments, the SRR plasmid also contains an SV40 origin of replication and optionally further expresses an SV40 large T antigen (eg. from an IRES sequence), allowing the SRR plasmid to be self-replicating and express the Anelloviridae family viral proteins at high levels. A cell containing both plasmids can thus produce packaged Anelloviridae family vector particles.

FIG. 77 is an illustration depicting the production of an Anelloviridae family vector particle in a mammalian cell (e.g., a MOLT-4 cell) comprising a minicircle containing the Anelloviridae family vector genetic element sequence, as well as an SRR plasmid encoding Anelloviridae family viral proteins, as described herein. Anelloviridae family vector particles containing genetic elements are made within the cell and can be isolated therefrom.

FIG. 78 is an illustration showing an exemplary process of nicking circular double stranded DNA (cdsDNA) at one or more sites (eg. five optimally spaced sites) and then using gel separation or exonuclease digest to produce circular single stranded DNA. The cdsDNA is nicked using an enzyme, e.g. Nt.BbvCI. The nicked DNA is then run on a gel to separate the smaller nicked fragments from the larger intact circular single stranded DNA. Separately, the nicked DNA can also be treated with an exonuclease, e.g. T7, to digest the nicked strand and produce circular single stranded DNA.

FIG. 79 is an illustration depicting an exemplary process for production of Anelloviridae family vector genetic element constructs (in the form of double-stranded DNA minicircles) in bacterial cells. Briefly, bacteria are transformed with a plasmid containing an Anelloviridae family vector genetic element sequence flanked by recombinase recognition sites, e.g., attP and attB sites as shown. A recombinase expressed by the bacterial strain (e.g., PhiC31) recombines the plasmid into a minicircle containing the Anelloviridae family vector genetic element sequence. The minicircle can be isolated from the bacterial cell and transfected into cells (e.g., MOLT-4 cells, 293 cells) with an SRR plasmid for production of Anelloviridae family vector particles or used in production of circular single stranded DNA (e.g., as described herein) for later encapsulation into Anelloviridae family vector particles.

FIG. 80 depicts a series of electrophoresis gels showing the isolation of minicircles from bacterial cells at various time scales after induction.

FIG. 81 is an illustration of an exemplary self-replicating rescue (SRR) plasmid comprising three expression cassettes. In the first cassette, a promoter drives expression of ORF1, ORF2, and ORF3 proteins, as well as SV40 Large T antigen via an internal ribosome entry site (IRES). The first cassette also includes an SV40 origin of replication. This cassette ensures that the SRR is self-replicating and provides the viral packaging proteins. A second cassette includes a promoter driving expression of a recombinase. A third cassette includes a promoter driving expression of anelloviral ORF1 separately from the first cassette.

FIG. 82 is an illustration depicting an exemplary self-replicating rescue (SRR) plasmid comprising four expression cassettes. In the first cassette, a promoter drives expression of ORF1, ORF2, and ORF3 proteins. In a second cassette, a promoter drives expression SV40 Large T antigen; this cassette also includes an SV40 origin of replication. A third cassette includes a promoter driving expression of a recombinase. A fourth cassette includes a promoter driving expression of anelloviral ORF1 separately from the first cassette.

FIG. 83 is an illustration depicting various arrangements of expression cassettes that may be included in a self-replicating rescue (SRR) plasmid as described herein. In some instances, 1, 2, 3, 4, or more cassettes may be included on the SRR plasmid in any order, provided, the SRRs contain at least one cassette expressing an SV40 Large T antigen and SV40 origin of replication, either through eg IRES or as a standalone cassette expressed from a promoter. In an example, a full ORF cassette can be included. In some instances, a mutant ORF cassette, for example with a stop codon in ORF1, can be included, with a separate expression cassette expressing ORF1. In some instances, a single ORF or recombinase cassette, for example, encoding an ORF1 protein or a recombinase, can be included. The direction/orientation of cassettes may also be varied.

FIG. 84 shows a gel of a 1 kb plus DNA ladder (lane 1), 400 ng of untreated DNA (lane 2), 400 ng of nicked DNA (lane 3), 1 μg of nicked and exonuclease-treated, unpurified DNA (lane 4), and 1 μg of nicked and exonuclease-treated, purified DNA (lane 5). The arrow indicates the expected banding position for the cssDNA.

FIG. 85 shows a gel of a 1 kb plus DNA ladder (lane 1), untreated DNA (lane 2), nicked DNA (lane 3), nicked and exonuclease-treated, unpurified DNA (lane 4), and nicked and exonuclease-treated, purified DNA (lane 5), all loaded with 500 ng of DNA. The arrow indicates the expected banding position for the cssDNA.

FIG. 86 shows the DNAse-protected viral genomes per liter (vgs/L) produced from MOLT-4 cells transfected with the genetic element construct encoding eGFP alone (“vector alone”; condition 1); the genetic element construct encoding eGFP, iCre-encoding plasmid, and the Ring19 SRR with ORF1 expression knocked out by a premature stop codo in its coding sequence (“vector+R19ORF1KO”; condition 2); the genetic element construct encoding eGFP, iCre-encoding plasmid, and Ring19 SRR (“vector+R19 SRR”; condition 3); the genetic element construct encoding eGFP, iCre-encoding plasmid, and Ring19 SRR which has the intron in the Ring19 NCR deleted but retains the remainder of the NCR and the native kozak sequence (“vector+R19Δintron1 SRR”; condition 4); and the genetic element construct encoding eGFP, iCre-encoding plasmid, and Ring19 SRR which contains the native kozak sequence but does not contain any other portion of the Ring19 NCR (“vector+R19Δexon1Δintron1 SRR”; condition 5). DNAse-protected viral genomes were detected with either eGFP or wild type Ring19—directed primers and probes.

FIG. 87A is a Western blot probed with an anti-Ring19 ORF1 antibody. Lane 1 is a protein ladder; lane 2 is from cells transfected with pRTx-3525 (“Ring19 full SRR”); lane 3 is from cells transfected with pRTx-3775 (“Ring19 ORF1 KO”); lane 4 is from cells transfected with pRTx-4534 (“Ring19 ORF1/1”); and lane 5 is an untransfected control.

FIG. 87B is a Western blot probed with an anti-Ring19 ORF2 antibody. Lane 1 is a protein ladder; lane 2 is from cells transfected with pRTx-3525 (“Ring19 full SRR”); lane 3 is from cells transfected with pRTx-3775 (“Ring19 ORF1 KO”); lane 4 is from cells transfected with pRTx-4534 (“Ring19 ORF1/1”); and lane 5 is an untransfected control.

FIG. 87C is a Western blot probed with an anti-Ring2 ORF1 antibody. Lane 1 is a protein ladder; lane 2 is from cells transfected with pRTx-3853 (“Ring2 full SRR”); lane 3 is from cells transfected with pRTx-3776 (“Ring2 ORF1 KO”); lane 4 is from cells transfected with pRTx-4463 (“Ring2 ORF1/1”); and lane 5 is untransfected control.

FIG. 87D is a Western blot probed with an anti-Ring2 ORF2 antibody. Lane 1 is a protein ladder; lane 2 is from cells transfected with pRTx-3853 (“Ring2 full SRR”); lane 3 is from cells transfected with pRTx-3776 (“Ring2 ORF1 KO”); lane 4 is from cells transfected with pRTx-4463 (“Ring2 ORF1/1”); and lane 5 is untransfected control.

FIG. 88 is a plot depicting the number of viral genomes detected by qPCR per liter of transfected cell culture. Cells were transfected with three plasmids: a self-replicating rescue (SRR) plasmid expressing ORF1 from Ring19, Ring2, Ring9, Ring25, Ring161, YYBYFW, or Ring34, a Cre expression plasmid, and a Ring19 genetic element construct. One control group was transfected with the Ring19 genetic element construct alone. Anellovectors with Ring19 genetic elements were produced at a higher level when co-transfected with SRRs from any of the tested Anellovectors than when the Ring19 genetic element construct is transfected alone.

FIG. 89 is a plot depicting the number of viral genomes detected by qPCR per liter of transfected cell culture. Cells were transfected with the Ring19 genetic element construct alone, the Ring19 genetic element construct, a Cre expression plasmid, and a Ring10 SRR, or the Ring19 genetic element construct, a Cre expression plasmid, and a Ring19 SRR. Anellovectors with Ring19 genetic elements were produced at a higher level when co-transfected with Ring10 or Ring19 SRR than when transfected alone.

FIG. 90 is a plot depicting the number of DNase-protected eGFP copies per fraction of cell lysate after an iodixanol linear gradient for two Anellovectors, Ring19_fCMV_eGFP (an Anellovector with Ring19 ORF1 packaging the Ring19-eGFP genetic element) and Ring2_fCMV_eGFP (an Anellovector with Ring2 ORF1 packaging the Ring19-eGFP genetic element).

FIG. 91 is a plot depicting the number of DNase-protected eGFP copies before and after concentration of viral material for two Anellovectors, Ring19_fCMV_eGFP (an Anellovector with Ring19 ORF1 packaging the Ring19-eGFP genetic element) and Ring2_fCMV_eGFP (an Anellovector with Ring2 ORF1 packaging the Ring19-eGFP genetic element).

FIG. 92 depicts a gel showing a Coomassie Stain on Ring19_fCMV_eGFP (an Anellovector with Ring19 ORF1 packaging the Ring19-eGFP genetic element) Material.

FIG. 93 depicts a gel showing a Coomassie Stain on Ring2_fCMV_eGFP (an Anellovector with Ring2 ORF1 packaging the Ring19-eGFP genetic element) Material.

FIGS. 94A-94F depict plots showing the quantification of eGFP in the posterior eye cup (FIG. 94A) and the retina (FIG. 94B), wild-type (WT) Ring2 (R2) genetic material in the posterior eye cup (FIG. 94C) and the retina (FIG. 94D), for wild-type (WT) Ring19 (R19) genetic material in the posterior eye cup (FIG. 94E) and the retina (FIG. 94F) by qPCR. Each dot represents one eye. WT Ring2 and WT Ring19 were not detected in either the posterior eye cup or the retina. n=3 eyes per group.

FIGS. 95A-95D are plots showing quantification of eGFP mRNA in the posterior eye cup by RT-qPCR (FIG. 95A) and RT-ddPCR (FIG. 95B), and quantification of eGFP mRNA in the retina by RT-qPCR (FIG. 95C) and RT-ddPCR (FIG. 95D). Each dot represents one eye. n=5 eyes per group.

FIGS. 96A-96L are representative fluorescence microscopy images of posterior eye cup tissue flatmounts. FIGS. 96A-96C show the eGFP channel, Texas red channel, and overlap of the two channels for the PBS control group, respectively. FIGS. 96D-96F show the eGFP channel, Texas red channel, and overlap of the two channels for the R2-eGFP group (Ring2 ORF1 packaging the Ring19-eGFP genetic element), respectively. FIGS. 96G-96I show the eGFP channel, Texas red channel, and overlap of the two channels for the R19-eGFP group (Ring19 ORF1 packaging the Ring19-eGFP genetic element), respectively. FIGS. 96J-96L show the eGFP channel, Texas red channel, and overlap of the two channels for the dose-matched AAV2-eGFP group, respectively. White arrows show eGFP positive cells. n=3 per group.

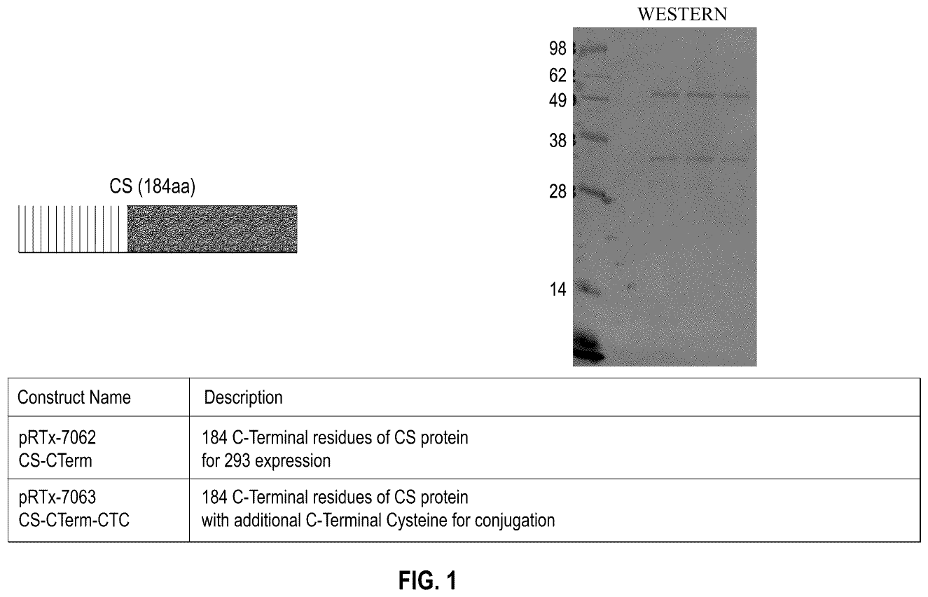

### DETAILED DESCRIPTION OF CERTAIN EMBODIMENTS

### Definitions

The present invention will be described with respect to particular embodiments and with reference to certain figures, but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “Anelloviridae family vector” refers to a vehicle derived from or similar to a virus of the Anelloviridae family (e.g., an Alphatorquevirus, Betatorquevirus, Gammatorquevirus, or chicken anemia virus), wherein the vehicle comprises a genetic element enclosed in a proteinaceous exterior (e.g., the genetic element is substantially protected from digestion with DNAse I by a proteinaceous exterior). In some embodiments, an Anelloviridae family vector comprises a genetic element derived from or highly similar to (e.g., at least 85%, 90%, 95%, 96%, 97%, 98%, 99%, or 100% identical to) that of an Alphatorquevirus, Betatorquevirus, Gammatorquevirus, or chicken anemia virus (CAV). In some embodiments, an Anelloviridae family vector comprises a proteinaceous exterior comprising a protein derived from or similar to (e.g., at least 50%, 60%, 70%, 75%, 80%, 85%, 90%, 95%, 96%, 97%, 98%, 99%, or 100% identical to) a capsid protein of an Alphatorquevirus, Betatorquevirus, Gammatorquevirus, or chicken anemia virus (e.g., an Alphatorquevirus ORF1, Betatorquevirus ORF1, Gammatorquevirus ORF1, or CAV VP1). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior or protected from digestion with DNAse I, e.g., prior to entry into a host cell. In some embodiments, the Anelloviridae family vector is purified, e.g., it is separated from its original source and/or substantially free (>50%, >60%, >70%, >80%, >90%) of other components. In some embodiments, the Anelloviridae family vector is capable of introducing the genetic element into a target cell (e.g., via infection). In some embodiments, the Anelloviridae family vector is an infective synthetic viral particle.

As used herein, the term “anellovector” refers to a vehicle comprising a genetic element, e.g., an episome, e.g., circular DNA, enclosed in a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). As used herein with respect to components or elements of an Anelloviridae family virus, the term “wild-type” generally refers to a naturally occurring version of the component or element (e.g., a genome sequence, coding sequence, non-coding element, polypeptide, or NCR sequence found in a naturally occurring Anelloviridae family virus, e.g., as described herein). In some embodiments, the proteinaceous exterior comprises an ORF1 molecule (e.g., an Anellovirus ORF1 protein), e.g., as described herein. In some embodiments, the proteinaceous exterior comprises a plurality of ORF1 molecules (e.g., an Anellovirus ORF1 protein), e.g., at least about 40, 45, 50, 55, 60, 65, or 70 ORF1 molecules. In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the anellovector is purified, e.g., it is separated from its original source and/or substantially free (>50%, >60%, >70%, >80%, >90%) of other components.

An anellovector may, in some embodiments, comprise a nucleic acid vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the nucleic acid vector is a viral vector or a naked nucleic acid. In some embodiments, the nucleic acid vector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3. In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. In embodiments, an anellovector comprises a genetic element described herein, e.g., comprises a genetic element comprising a promoter, a sequence encoding a therapeutic effector, and a capsid binding sequence.

The term “Anelloviridae family vector genetic element sequence,” as used herein, refers to a sequence of nucleotides (e.g., within a nucleic acid molecule, which in some instances may comprise additional sequences) having the nucleic acid sequence of the genetic element of an Anelloviridae family vector. Likewise, as used herein, “Anellovector genetic element sequence” refers to a sequence of nucleotides (e.g., within a nucleic acid molecule, which in some instances may comprise additional sequences) having the nucleic acid sequence of the genetic element of an Anellovector. Generally, an Anelloviridae family vector (e.g., Anellovector) genetic element sequence can, if excised from any surrounding nucleic acid sequence and converted to a single-stranded circular DNA form, be encapsulated within a proteinaceous exterior comprising an ORF1 molecule to form an Anelloviridae family vector (e.g., Anellovector) as described herein. In some embodiments, an Anelloviridae family vector (e.g., Anellovector) genetic element sequence is present in a single-stranded DNA, such as a genetic element of an Anelloviridae family vector (e.g., Anellovector) as described herein. In some embodiments, an Anelloviridae family vector (e.g., Anellovector) genetic element sequence is present in a double-stranded DNA, such as a plasmid or minicircle, e.g., as described herein. In some embodiments, an Anelloviridae family vector (e.g., Anellovector) genetic element sequence is present as a positive strand sequence. In some embodiments, an Anelloviridae family vector (e.g., Anellovector) genetic element sequence is present as a negative strand sequence.

As used herein, the term “anelloVLP” refers to a vehicle (e.g., a virus-like particle) comprising a proteinaceous exterior and an effector (e.g., an exogenous effector). In some instances, an anelloVLP does not comprise a substantial amount of a nucleic acid. In some embodiments, the proteinaceous exterior comprises an ORF1 molecule (e.g., an Anellovirus ORF1 protein), e.g., as described herein. In some embodiments, the proteinaceous exterior comprises a plurality of ORF1 molecules (e.g., an Anellovirus ORF1 protein), e.g., at least about 40, 45, 50, 55, 60, 65, or 70 ORF1 molecules. In some embodiments, the effector is enclosed in the proteinaceous exterior. In some embodiments, the effector is on the surface of the proteinaceous exterior (e.g., comprised in a surface moiety as described herein). In some embodiments, the anelloVLP does not comprise a polynucleotide of greater than 50, 100, 150, 200, 300, 400, 500, 600, 700, 800, 900, 1000, 1100, 1200, 1300, 1400, 1500, 1600, 1700, 1800, 1900, or 2000 nucleotides in length. In some embodiments, the anelloVLP does not comprise a polynucleotide comprising an Anellovirus 5′ UTR or Anellovirus origin of replication. In some embodiments, the anelloVLP does not comprise a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein).

As used herein, the term “Anellovirus non-coding region (NCR)” refers to a sequence of an untranslated region of an Anellovirus genome sequence that extends from just upstream of the ORF2 start codon to the untranslated region of the Anellovirus genome sequence just downstream of the ORF3 stop codon in a circular genome. The Anellovirus NCR may comprise the “Anellovirus 5′ NCR sequence” and “Anellovirus 3′ NCR sequence.” In some embodiments, the Anellovirus NCR sequence is contiguous. In some embodiments, the Anellovirus NCR sequence is non-contiguous. In some embodiments, the portions of a non-contiguous Anellovirus NCR sequence are separated by a heterologous insertion (e.g., comprising a recombinase hybrid site, e.g., as described herein). In some embodiments, the Anellovirus NCR sequence comprises a sequence found in a wild-type Anellovirus, and in other embodiments, the Anellovirus NCR comprises one or more mutations relative to the closest Anellovirus sequence. In some embodiments, the Anellovirus NCR is comprised by a nucleic acid molecule that does not comprise Anellovirus ORF2 or ORF3 coding sequences. In some instances, the Anellovirus NCR refers to the sense strand, the antisense strand, or a double-stranded DNA comprising both the sense strand and antisense strands. In some instances, a listing of an Anellovirus NCR sequence herein can refer to the sequence listed and/or its reverse complement.

As used herein, the term “Anellovirus 5′ NCR” refers to a sequence of an untranslated region of an Anellovirus genome sequence that extends from just upstream of the ORF2 start codon through the 5′ UTR conserved domain to the Anellovirus 3′ NCR sequence, and sequences with homology thereto. In some embodiments, an Anellovirus 5′ NCR sequence comprises origin of replication activity. In some embodiments, the Anellovirus 5′ NCR sequence is contiguous. In some embodiments, the Anellovirus 5′ NCR sequence is non-contiguous. In some embodiments, the portions of a non-contiguous Anellovirus 5′ NCR sequence are separated by a heterologous insertion (e.g., comprising a recombinase hybrid site, e.g., as described herein). In some embodiments, the Anellovirus 5′ NCR sequence comprises a sequence found in a wild-type Anellovirus, and in other embodiments, the Anellovirus 5′ NCR comprises one or more mutations relative to the closest Anellovirus sequence. In some instances, the Anellovirus 5′ NCR refers to the sense strand, the antisense strand, or a double-stranded DNA comprising both the sense strand and antisense strands. In some instances, a listing of an Anellovirus 5′ NCR sequence herein can refer to the sequence listed and/or its reverse complement. In a circular genetic element, the Anellovirus 5′ NCR and Anellovirus 3′ NCR may be directly adjacent to each other (e.g., to form an Anellovirus NCR). Exemplary dividing points between the Anellovirus 5′ NCR and the Anellovirus 3′ NCR are shown, e.g., as described herein.

As used herein, the term “Anellovirus 3′ NCR” refer to a sequence of an untranslated region of an Anellovirus genome sequence that extends from just downtream of the ORF3 stop codon through the GC-rich region to the Anellovirus 5′ NCR sequence, and sequences with homology thereto. In some embodiments, the Anellovirus 3′ NCR sequence is contiguous. In some embodiments, the Anellovirus 3′ NCR sequence is non-contiguous. In some embodiments, the portions of a non-contiguous Anellovirus 3′ NCR sequence are separated by a heterologous insertion (e.g., comprising a recombinase hybrid site, e.g., as described herein). In some embodiments, the Anellovirus 3′ NCR sequence comprises a sequence found in a wild-type Anellovirus, and in other embodiments, the Anellovirus 3′ NCR comprises one or more mutations relative to the closest Anellovirus sequence. In some instances, the Anellovirus 3′ NCR refers to the sense strand, the antisense strand, or a double-stranded DNA comprising both the sense strand and antisense strands. In some instances, a listing of an Anellovirus 3′ NCR sequence herein can refer to the sequence listed and/or its reverse complement.

As used herein, the term “Anellovirus GC-rich region” refers to a wild-type or engineered sequence that has an activity and a structural feature of a GC-rich region of a wild-type Anellovirus, or a functional fragment thereof. In some embodiments, the functional fragment has a length of at least 20, 30, 40, 50, 60, 70, 80, 90, or 100 nucleotides. Typically, the negative strand comprising the Anellovirus GC-rich region is packaged into a particle (e.g., an Anelloviridae family vector) as described herein. In some embodiments, the Anellovirus GC-rich region is a wild-type Anellovirus GC-rich region. In some embodiments, the Anellovirus GC-rich region is an engineered Anellovirus GC-rich region having a nucleic acid sequence with at least one difference relative to the closest wild-type Anellovirus GC-rich region sequence.

As used herein, the term “Anellovirus 5′ UTR conserved domain” refers to a wild-type or engineered sequence that has an activity and a structural feature of an Anellovirus 5′ UTR conserved domain of a wild-type Anellovirus, or a functional fragment thereof. In some embodiments, the functional fragment has a length of at least 15, 20, 30, 40, 50, 60, or 70 nucleotides. Typically, the negative strand comprising the Anellovirus 5′ UTR conserved domain is packaged into a particle (e.g., an Anelloviridae family vector) as described herein. In some embodiments, the Anellovirus 5′ UTR conserved domain is a wild-type Anellovirus 5′ UTR conserved domain. In some embodiments, the Anellovirus 5′ UTR conserved domain is an engineered Anellovirus 5′ UTR conserved domain having a nucleic acid sequence with at least one difference relative to the closest wild-type Anellovirus 5′ UTR conserved domain sequence.

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

The term “deletion,” as used herein with respect to an amino acid sequence or a nucleic acid sequence, refers to a portion of a sequence that is absent relative to a reference sequence. In some embodiments, a deletion is actively removed from the sequence (e.g., by cleavage and/or by an enzyme). In some embodiments, the sequence is produced de novo without the deletion sequence (e.g., a nucleic acid molecule synthesized de novo without the deletion sequence, a nucleic acid molecule produced using a template sequence in which the deletion sequence has already been removed, or a polypeptide translated from a nucleic acid sequence that does not encode the deletion sequence).

The term “disassembly,” as used herein with respect to a particle, such as a virus-like particle (VLP), or a proteinaceous exterior, refers to disassociating one or more components of the particle (e.g., a capsid protein, e.g., an ORF1 molecule as described herein) from the remainder of the particle. In some instances, disassembly of a particle (e.g., a VLP) comprises separating enough of the ORF1 molecules from each other that they no longer form a proteinaceous exterior. In some instances, a ORF1 molecules separated from each other via disassembly of a particle form capsomers (e.g., decameric capsomers), e.g., as described herein. In some embodiments, disassembly reduces the particle to individual monomers. In some embodiments, after disassembly, multimers, e.g., decamers, monomers, and/or pentamers remain. In some instances, disassembly comprises denaturation of protein complexes of the particle (e.g., breaking noncovalent bounds between ORF1 molecules in the proteinaceous exterior). In some instances, disassembly is driven by a denaturant as described herein

The term “in vitro assembly,” as used herein with respect to an Anelloviridae family vector (e.g., anellovector) or an anelloVLP, refers to the formation of a proteinaceous exterior comprising an ORF1 molecule, wherein the formation does not take place inside of a cell (e.g., takes place in a cell-free system such as a cell-free suspension, a lysate, or a supernatant). In some instances, in vitro assembly of an Anelloviridae family vector (e.g., anellovector) comprises enclosure, outside of a cell, of a genetic element (e.g., as described herein) within the proteinaceous exterior. In some instances, in vitro assembly of an anelloVLP comprises association, outside of a cell, of an effector (e.g., an exogenous effector, e.g., as described herein) with the proteinaceous exterior (e.g., enclosed within the proteinaceous exterior). In vitro assembly of a proteinaceous exterior may occur, in some instances, under conditions suitable for multimerization of a plurality of ORF1 molecules (e.g., nondenaturing conditions), e.g., to form a multimer of more than 10, 11, 12, 13, 14, 15, 16, 17, 18, 19, or 20 ORF1 molecules. In some instances, in vitro assembly results in the formation of a proteinaceous exterior comprising at least about 20, 30, 40, 50, or 60 ORF1 molecules, or about 20-30, 30-40, 40-50, 50-60, or 60-70 ORF1 molecules). In some instances, the proteinaceous exterior is formed from ORF1 molecules that were produced in a cell and then purified therefrom. In some instances, the in vitro assembly takes place in a solution free of cells or constituents thereof. In other instances, the in vitro assembly takes place in a solution comprising cell debris (e.g., from lysed cells). In some instances, the in vitro assembly takes place in a solution substantially free of cellular nucleic acid molecules (e.g., genomic DNA, mitochondrial DNA, mRNA, and/or noncoding RNA from a cell).

As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “first” or “second” Anellovirus genomic sequence, as used herein, each refer to a distinct nucleic acid sequence of a genetic element of a virus of the Anelloviridae family or of an Anelloviridae family vector. In some embodiments, the first or second Anellovirus genomic sequence is a wild-type Anellovirus genomic sequence. In some embodiments, the first or second Anellovirus genomic sequence is a synthetic Anellovirus genomic sequence. The first and second Anellovirus genomic sequences comprise at least one nucleotide difference (e.g., substitution, deletion, or insertion) relative to each other.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid sequence, generally in an anellovector. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, the term “ORF1 molecule” refers to a polypeptide having an activity and/or a structural feature of an Anellovirus ORF1 protein (e.g., an Anellovirus ORF1 protein as described herein, e.g., as listed in Tables A1-A32), or a functional fragment thereof. An ORF1 molecule may, in some instances, comprise one or more of (e.g., 1, 2, 3 or 4 of): a first region comprising at least 60% basic residues (e.g., at least 60% arginine residues), a second region compising at least about six beta strands (e.g., at least 4, 5, 6, 7, 8, 9, 10, 11, or 12 beta strands), a third region comprising a structure or an activity of an Anellovirus structural N22 domain (e.g., as described herein, e.g., an structural N22 domain from an Anellovirus ORF1 protein as described herein), and/or a fourth region comprising a structure or an activity of an Anellovirus structural C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid (e.g., as listed in any of Tables N1-N32). An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein), e.g., an ORF1 protein having the amino acid sequence as listed in Tables A1-A32, or as encoded by the ORF1 gene as listed in any of Tables N1-N32.

As used herein, the term “wild-type ORF1 gene” refers to a nucleic acid sequence that encodes a wild-type ORF1 protein (e.g., as described herein, e.g., a wild-type Ring2 ORF1 protein or a wild-type Ring19 ORF1 protein). In some embodiments, a wild-type ORF1 gene comprises one or more non-coding nucleic acid sequences (e.g., a 5′ UTR or a 3′ UTR). In some embodiments, a wild-type ORF1 gene is a codon-optimized sequence encoding a wild-type ORF1 protein.

The term “ORF1 domain,” as used herein with respect to an ORF1 molecule, refers to the portion of the ORF1 molecule having the structure or function of an Anellovirus ORF1 protein. The ORF1 domain is generally capable of forming a multimer with other copies of the ORF1 domain (e.g., in other ORF1 molecules), or with other ORF1 molecules, e.g., to form a proteinaceous exterior (e.g., of an anellovector or anelloVLP as described herein). In some instances, the ORF1 molecule may comprise one or more additional domains other than the ORF1 domain (for example, a domain comprising or attached to a surface effector, e.g., as described herein). In some instances, the amino acid sequence of an ORF1 domain comprises an insertion (e.g., an insertion encoding a surface moiety or a domain capable of binding to a surface moiety), e.g., between the N-terminal end and C-terminal end of the ORF1 domain. In certain instances, the insertion does not substantially disrupt the structure and/or function of the ORF1 domain, e.g., such that the ORF1 domain remains capable of forming a multimer with other ORF1 domains or ORF1 molecules. The position within the ORF1 domain sequence into which the insertion is made is referred to herein as the “insertion point.” An insertion can be made into an ORF1 domain by any genetic or polypeptide engineering method known in the art. In some embodiments, an ORF1 molecule consists of an ORF1 domain. In other embodiments, an ORF1 molecule comprises an ORF1 domain and a heterologous domain (e.g., a surface moiety as described herein). In some embodiments, an ORF1 domain is connected to a surface moiety by a polypeptide linker region.

As used herein, the term “ORF2 molecule” refers to a polypeptide having an activity and/or a structural feature of an Anellovirus ORF2 protein (e.g., an Anellovirus ORF2 protein as described herein, e.g., as listed in Tables A1-A32), or a functional fragment thereof. An “Anellovirus ORF2 protein,” as used herein, refers to an ORF2 protein encoded by an Anellovirus genome (e.g., a wild-type Anellovirus genome, e.g., as described herein), e.g., an ORF2 protein having the amino acid sequence as listed in Tables A1-A32, or as encoded by the ORF2 gene as listed in any of Tables N1-N32.

As used herein, the term “VP1 molecule” refers to a polypeptide having an activity and/or a structural feature of a CAV VP1 protein (e.g., a CAV VP1 protein as described herein, or a functional fragment thereof. A VP1 molecule may, in some instances, comprise a polypeptide encoded by a CAV VP1 nucleic acid. A VP1 molecule may, in some instances, further comprise a heterologous sequence, e.g., from a CAV VP1 protein, e.g., as described herein. In some embodiments, a VP1 molecule is encoded by a CAV genome (e.g., a wild-type CAV genome, e.g., as described herein). In some embodiments, a VP1 molecule is a polypeptide encoded by a CAV VP1 nucleic acid (e.g., a VP1 gene, e.g., as described herein). In some embodiments, a VP1 molecule is a splice variant or comprises a post-translational modification.

As used herein, the term “VP2 molecule” refers to a polypeptide having an activity and/or a structural feature of a CAV VP2 protein (e.g., a CAV VP2 protein as described herein, or a functional fragment thereof. In some embodiments, a VP2 molecule is encoded by a CAV genome (e.g., a wild-type CAV genome, e.g., as described herein). In some embodiments, a VP2 molecule is a polypeptide encoded by a CAV VP2 nucleic acid (e.g., a VP2 gene, e.g., as described herein). In some embodiments, a VP2 molecule is a splice variant or comprises a post-translational modification.

As used herein, the term “Apoptin molecule” and “VP3 molecule” are used interchangeably and refer to a polypeptide having an activity and/or a structural feature of a CAV Apoptin protein (e.g., a CAV Apoptin protein as described herein, or a functional fragment thereof. In some embodiments, an Apoptin molecule is encoded by a CAV genome (e.g., a wild-type CAV genome, e.g., as described herein). In some embodiments, an Apoptin molecule is a polypeptide encoded by a CAV Apoptin nucleic acid (e.g., an Apoptin gene). In some embodiments, an Apoptin molecule is a splice variant or comprises a post-translational modification.

As used herein, the term “CAV capsid polypeptide” refers to a polypeptide present in the capsid of a wild-type CAV, or a polypeptide having an activity and/or a structural feature of said polypeptide. In some embodiments, the CAV capsid polypeptide is a VP1 molecule.

As used herein, the term “VP1 nucleic acid” refers to a nucleic acid that encodes a VP1 molecule, or the reverse complement thereof. The nucleic acid may be single stranded or double stranded. In some embodiments, the VP1 nucleic acid comprises a CAV VP1 gene, e.g., as described herein. A “VP1 gene” generally refers to a nucleic acid sequence encoding a wild-type VP1 molecule, or the reverse complement thereof. In some embodiments, a VP1 gene comprises a sense strand. In some embodiments, a VP1 gene comprises an antisense strand. In some embodiments, a VP1 gene is double-stranded.

As used herein, the term “VP2 nucleic acid” refers to a nucleic acid that encodes a VP2 molecule, or the reverse complement thereof. The nucleic acid may be single stranded or double stranded. In some embodiments, the VP2 nucleic acid comprises a CAV VP2 gene, e.g., as described herein. A “VP2 gene” generally refers to a nucleic acid sequence encoding a wild-type VP2 molecule, or the reverse complement thereof. In some embodiments, a VP2 gene comprises a sense strand. In some embodiments, a VP2 gene comprises an antisense strand. In some embodiments, a VP2 gene is double-stranded.

As used herein, the term “Apoptin nucleic acid” and “VP3 nucleic acid” are used interchangeably, and refer to a nucleic acid that encodes a Apoptin molecule, or the reverse complement thereof. The nucleic acid may be single stranded or double stranded. In some embodiments, the Apoptin nucleic acid comprises a CAV Apoptin gene, e.g., as described herein. An “Apoptin gene” or “VP3 gene” generally refers to a nucleic acid sequence encoding a wild-type Apoptin molecule, or the reverse complement thereof. In some embodiments, an Apoptin gene comprises a sense strand. In some embodiments, an Apoptin gene comprises an antisense strand. In some embodiments, an Apoptin gene is double-stranded.

As used herein, the term “CAV genome sequence” refers to a nucleic acid sequence comprising a full-length genome sequence from a wild-type CAV, e.g., as described herein, or a sequence having at least 70%, 75%, 80%, 85%, 90%, 95%, 96%, 97%, 98%, 99%, or 100% sequence identity thereto. In some embodiments, a CAV genome comprises a CAV genome sequence as described herein (e.g., a wild-type CAV genome sequence, e.g., as listed in any of Tables N3-N4).

As used herein, the term “CAV NCR” refers to a sequence of an untranslated region of an Anellovirus genome sequence that extends from just upstream of the VP2 start codon to the untranslated region of the Anellovirus genome sequence just downstream of the VP1 stop codon in a circular genome (e.g., comprising the sequence of a 5′ NCR and/or a 3′ NCR) from a CAV (e.g., a wild-type CAV, e.g., as described herein, e.g., as listed in Table N3-N4), or a sequence having at least 70%, 75%, 80%, 85%, 90%, 95%, 96%, 97%, 98%, 99%, or 100% identity thereto. In some embodiments, the CAV NCR sequence is contiguous. In some embodiments, the CAV NCR sequence is non-contiguous. In some embodiments, the portions of a non-contiguous CAV NCR sequence are separated by a heterologous insertion (e.g., comprising a recombinase hybrid site, e.g., as described herein). In some embodiments, the CAV NCR sequence comprises a sequence found in a wild-type CAV, and in other embodiments, the CAV NCR comprises one or more mutations relative to the closest CAV sequence. In some embodiments, the CAV NCR is comprised by a nucleic acid molecule that does not comprise CAV VP1 or VP2 coding sequences.

As used herein, the term “particle” refers to a vehicle having a diameter of less than 100 nm (e.g., about 20-25, 25-30, 30-35, or 35-40 nm) comprising a proteinaceous exterior. In some instances, the particle comprises a plurality of ORF1 molecules. The proteinaceous exterior of the particle generally forms an enclosure capable of limiting or preventing movement of certain molecules between the inside and outside of the proteinaceous exterior. In some embodiments, gaps or discontinuities (e.g., that render the proteinaceous exterior permeable to water, ions, peptides, or small molecules) may be present in the proteinaceous exterior. In certain embodiments, the gaps or discontinuities are of a sufficiently small size (e.g., diameter) that the proteinaceous exterior limits or prevents one or more large macromolecules (e.g., peptides, polypeptides, polynucleotides, lipids, or polysaccharides) from passing through the proteinaceous exterior.

As used herein, the term “proteinaceous exterior” refers to an exterior component that is predominantly (e.g., >50%, >60%, >70%, >80%, >90%) protein.

As used herein, the term “recombinase hybrid site” refers to a DNA site having a sequence that, when in double stranded form, is capable of being produced by a site-specific recombinase that recombines two recombinase recognition sites. No particular process of making is implied: a recombinase hybrid site can be produced by a site specific recombinase or another method, such as DNA replication of an existing sequence. In some embodiments, a single stranded DNA that comprises a recombinase hybrid site was produced by a method wherein a site-specific recombinase generated a double stranded DNA comprising a recombinase hybrid site, followed by conversion of the double stranded DNA to a single stranded DNA. In some embodiments (e.g., with Cre recombinase) the recombinase hybrid site has the same sequence as one of the corresponding recombinase recognition sites. In some embodiments (e.g., with Bxb1 recombinase), the recombinase hybrid site has a different site from either of the two corresponding recombinase recognition sites. In some embodiments, the recombinase hybrid site is a loxP site, an attL site, or an attR site.

As used herein, the term “recombinase recognition site” refers to a DNA site having a sequence that is capable of being recognized by a site-specific recombinase and recombined with a second recombinase recognition site, thereby producing a recombinase hybrid site. In some embodiments, the two recombinase recognition sites recognized by the recombinase have the same sequence, and in other emobdiments, they have different seuqences. In some embodiments, the recombinase recognition site is a loxP site, an attB site, or an attP site.

As used herein, the term “regulatory nucleic acid” refers to a nucleic acid sequence that modifies expression, e.g., transcription and/or translation, of a DNA sequence that encodes an expression product. In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

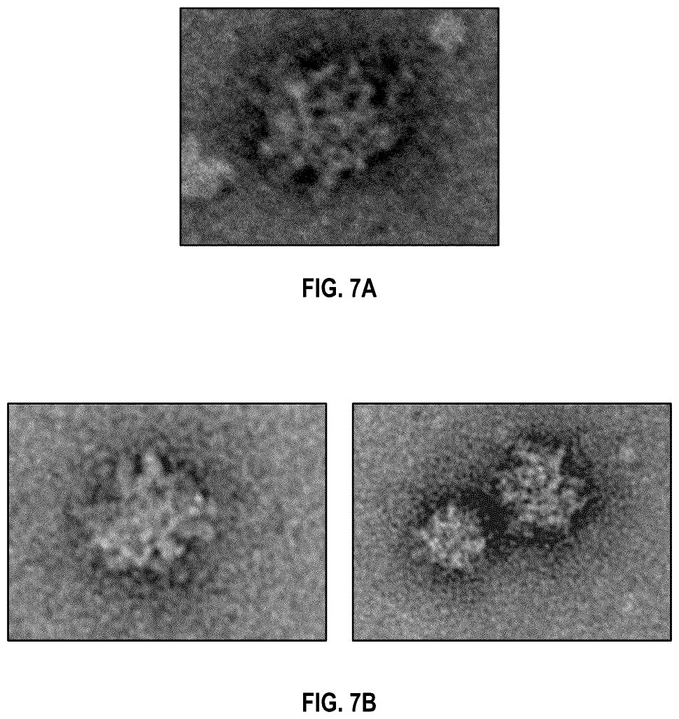

When viewed by electron microscopy, anellovector or anelloVLP particles typically adopt one of two conformations: a symmetrical morphology (e.g., as exemplified in FIG. 7A) and an asymmetrical, or less symmetrical, morphology (e.g., as exemplified in FIG. 7B). Accordingly, the term “symmetrical morphology,” as used herein with respect to anellovector or anelloVLP particle morphology, refers to a particle having a shape that is predominantly symmetrical. The particle having symmetrical morphology may, in some instances, be approximately round. The particle having symmetrical morphology may, in some instances, not be perfectly circular or spherical (e.g., may be ovoid). In some instances, the particle having symmetrical morphology may include one or more deviations from a circular or spherical shape (e.g., one or more protrusions or indentations from its surface).

As used herein, the term “structural arginine-rich region” refers to a domain of an Anellovirus ORF1 molecule having a structural arginine-rich region sequence as listed in any of Tables B1-1 to B1-12, or a corresponding sequence in another ORF1 molecule.

As used herein, the term “structural jelly-roll region” refers to a domain of an Anellovirus ORF1 molecule having a structural jelly-roll region sequence as listed in any of Tables B1-1 to B1-12, or a corresponding sequence in another ORF1 molecule.

As used herein, the term “structural N22 domain” refers to a domain of an Anellovirus ORF1 molecule having a structural N22 domain sequence as listed in any of Tables B1-1 to B1-12, or a corresponding sequence in another ORF1 molecule.

As used herein, the term “structural C-terminal domain region” refers to a domain of an Anellovirus ORF1 molecule having a structural C-terminal domain sequence as listed in any of Tables B1-1 to B1-12, or a corresponding sequence in another ORF1 molecule.

As used herein, the term “jelly-roll B—H strands subdomain” refers to a domain of an Anellovirus ORF1 molecule having a jelly-roll B—H strands subdomain sequence as listed in any of Tables B1-1 to B1-12, or a corresponding sequence in another ORF1 molecule.

As used herein, the term “P1 domain” generally refers to a noncontiguous domain comprising a P1-1 subdomain and a P1-2 subdomain, e.g., of an Anellovirus ORF1 molecule.

As used herein, the term “P1-1 subdomain” refers to a domain of an Anellovirus ORF1 molecule having a P1-1 domain sequence as listed in any of Tables B1-1 to B1-12, or a corresponding sequence in another ORF1 molecule.

As used herein, the term “P2 domain” refers to a domain of an Anellovirus ORF1 molecule having a P2 domain sequence as listed in any of Tables B1-1 to B1-12, or a corresponding sequence in another ORF1 molecule.

As used herein, the term “P1-2 subdomain” refers to a domain of an Anellovirus ORF1 molecule having a P1-2 subdomain sequence as listed in any of Tables B1-1 to B1-12, or a corresponding sequence in another ORF1 molecule.

As used herein, the term “jelly-roll I strand subdomain” refers to a domain of an Anellovirus ORF1 molecule having a jelly-roll I strand subdomain sequence as listed in any of Tables B1-1 to B1-12, or a corresponding sequence in another ORF1 molecule.

As used herein, the term “mutant ORF1,” as applied to a particular domain or region of an ORF1 molecule, refers to a non-naturally occurring ORF1 domain or region comprising at least one sequence difference (e.g., addition, deletion, or substitution) relative to the closest naturally-occurring ORF1 domain or region sequence. For instance, a “mutant ORF1 structural jelly-roll region” comprises at least one sequence difference (e.g., addition, deletion, or substitution) relative to the closest naturally-occurring Anellovirus ORF1 structural jelly-roll region.

As used herein, a “substantially non-pathogenic” organism, particle, or component, refers to an organism, particle (e.g., a virus or an anellovector, e.g., as described herein), or component thereof that does not cause or induce a detectable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a detectable immune response. In some embodiments, the substantially non-immunogenic anellovector does not produce a detectable immune response against a protein comprising an amino acid sequence or encoded by a nucleic acid sequence shown in any of Tables N1-N32. In some embodiments, an immune response (e.g., an undesired or untargeted immune response) is detected by assaying antibody presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

As used herein, the term “surface moiety” refers to a moiety for which at least a portion is exposed on the exterior surface of a particle (e.g., exposed to the solution surrounding the particle). The surface moiety is generally attached, directly or indirectly, to a component of the proteinaceous exterior of the particle (e.g., an ORF1 molecule). In some instances, the surface moiety is covalently attached to the component of the proteinaceous exterior of the particle (e.g., the ORF1 molecule). In some instances, the surface moiety is noncovalently attached to the component of the proteinaceous exterior of the particle (e.g., the ORF1 molecule). In some instances, the surface moiety is bound to a binding moiety that is in turn attached (e.g., covalently or noncovalently) to the component of the proteinaceous exterior of the particle (e.g., the ORF1 molecule). In some instances, the surface moiety is comprised in an ORF1 molecule (e.g., is a heterologous domain of an ORF1 molecule). In some instances, a surface moiety is exogenous relative to an Anellovirus (e.g., the Anellovirus from which the ORF1 molecule was derived and/or an Anellovirus for which the ORF1 protein has at least 50%, 60%, 70%, 75%, 80%, 85%, 90%, 95%, 96%, 97%, 98%, 99%, or 100% sequence identity to the ORF1 molecule). In some instances, a surface moiety is exogenous relative a target cell (e.g., a mammalian cell, e.g., a human cell) to be infected by the particle.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

As used herein, the term “virome” refers to viruses in a particular environment, e.g., a part of a body, e.g., in an organism, e.g. in a cell, e.g. in a tissue.

This invention relates generally to Anelloviridae family vectors (e.g., anellovectors), e.g., synthetic Anelloviridae family vectors (e.g., anellovectors), and uses thereof. The present disclosure provides Anelloviridae family vectors (e.g., anellovectors), compositions comprising Anelloviridae family vectors (e.g., anellovectors), and methods of making or using Anelloviridae family vectors (e.g., anellovectors). Anelloviridae family vectors (e.g., anellovectors) are generally useful as delivery vehicles, e.g., for delivering a therapeutic agent to a eukaryotic cell. Generally, an Anelloviridae family vector (e.g., anellovector) will include a genetic element comprising a nucleic acid sequence (e.g., encoding an effector, e.g., an exogenous effector or an endogenous effector) enclosed within a proteinaceous exterior. An Anelloviridae family vector (e.g., anellovector) may include one or more deletions of sequences (e.g., regions or domains as described herein) relative to an Anellovirus sequence (e.g., as described herein). Anelloviridae family vectors (e.g., anellovectors) can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

TABLE OF CONTENTS

```
I. Anelloviridae family vectors (e.g., anellovectors) and AnelloVLPs
  A. Anelloviridae family vectors (e.g., Anellovectors)B. ORF1 molecules
    i. Anellovirus ORF1 molecule domain structures
    ii. Structural arginine-rich region deletions and truncations
    iii. Structural C-terminal domain deletions and truncations
    iv. Chimeric ORF1 molecules
      (a) N-terminal insertions (e.g., structural arginine-rich region swaps)
      (b) P1/P2 domain swaps
      (c) Other domain mutants
  C. ORF2 molecules
  D. Genetic elements
  E. Protein binding sequences
  F. 5′ UTR Regions
  G. GC-rich regions
  H. Effectors
  I. Proteinaceous exterior
  J. Surface moieties
    i. Click chemistry
      (a) Mutations of surface lysines
      (b) Mutations of surface cysteines
    ii. Polypeptides
    iii. Genetic grafting of ORF1 to a surface moiety
    iv. X-fold symmetry
    v. Nucleic acid molecules
    vi. Small molecules
    vii. Vaccines/Antigens
    viii. Ligands
II. Compositions and Methods for Making Anellovectors and AnelloVLPs
  A. Components and Assembly of Anellovectors and AnelloVLPs
    i. ORF1 molecules, e.g., for assembly of anellovectors
    ii. ORF2 molecules, e.g., for assembly of anellovectors
    iii. Production of protein components
      (a) Baculovirus expression systems
      (b) Insect cell systems
      (c) Mammalian cell systems
  B. Recombinase-based production of genetic elements and Anelloviridae family vectors
      (a) Three-plasmid systems
      (b) Two-plasmid systems
      (c) Self-replicating rescue (SRR) constructs (e.g., SRR plasmids)
      (d) Bacterial minicircle systems
      (e) Conversion of minicircles to cssDNA genetic elements
      (f) Exemplary site-specific recombinases and recombinase recognition sites
  C. Genetic Element Constructs, e.g., for assembly of Anellovectors
    i. Plasmids
    ii. Constructs comprising recombinase recognition sequences
    iii. Small circular nucleic acid constructs
    iv. Cis/trans constructs
    v. Expression cassettes
    vi. Design and production of a genetic element construct
  D. Effectors
  E. Host Cells
    i. Introduction of genetic elements into host cells
    ii. Methods for providing protein(s) in cis or trans
    iii. Exemplary cell types
  F. Culture Conditions
  G. Harvest
  H. In vitro assembly methods for anellovectors
  I. In vitro assembly for anelloVLPs
  J. Enrichment and Purification
III. Vectors
IV. Compositions
V. Host cells
VI. Methods of use
  A. Methods of Treatment (e.g., for diseases or disorders of the eye)
VII. Methods of production
VIII. Administration/ Delivery
  A. Ocular delivery
  B. Redosing
```

### I. Anelloviridae Family Vectors (e.g., Anellovectors) and AnelloVLPs

In some aspects, the invention described herein comprises compositions and methods of using and making an Anelloviridae family vector (e.g., an anellovector), Anelloviridae family VLPs (e.g., anelloVLPs), and preparations and therapeutic compositions relating to same.

### Anelloviridae Family Vectors (e.g., Anellovectors)

Provided herein are particles for drug delivery comprising one or more polypeptides or nucleic acid elements from an Anelloviridae family virus (e.g., an Anellovirus or CAV, e.g., as described herein). It is noted that descriptions herein referring to anellovectors are likewise applicable to other Anelloviridae family vectors, such as vectors based on CAV. Similarly, descriptions herein referring to Anelloviruses (or components thereof, such as nucleic acid sequences or polypeptides) are applicable to other Anelloviridae family viruses, such as CAV (or components thereof). In some embodiments, the anellovector has a sequence, structure, and/or function that is based on an Anellovirus (e.g., an Anellovirus as described herein, e.g., an Anellovirus comprising a nucleic acid or polypeptide comprising a sequence as shown in any one of Tables A1-A32 or N1-N32), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as listed in any one of Tables N1-N32); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In some embodiments, the promoter element is selected from an RNA polymerase II-dependent promoter, an RNA polymerase III-dependent promoter, a PGK promoter, a CMV promoter, an EF-1α promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In some embodiments, the promoter element comprises a TATA box. In some embodiments, the promoter element is endogenous to a wild-type Anellovirus, e.g., as described herein.

In some embodiments, the genetic element comprises one or more of the following characteristics: single-stranded, circular, negative strand, and/or DNA. In some embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The anellovectors, compositions comprising anellovectors, methods using such anellovectors, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce anellovectors which can then be used to deliver an effector to cells (e.g., animal cells, e.g., human cells or non-human animal cells such as pig or mouse cells). In embodiments, the effector can silence expression of a factor such as an interferon. The examples further describe how anellovectors can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In some embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, introduction of the anellovector, or genetic element comprised therein, decreases level of interferon produced by the cell. In some embodiments, introduction of the anellovector, or genetic element comprised therein, into a cell modulates (e.g., increases or decreases) a function of the cell. In some embodiments, introduction of the anellovector, or genetic element comprised therein, into a cell modulates (e.g., increases or decreases) the viability of the cell. In some embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus) (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anelloviridae family virus (e.g., Anellovirus) (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus) (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anelloviridae family virus (e.g., Anellovirus) (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anelloviridae family virus (e.g., Anellovirus) (e.g., as described herein).

### AnelloVLPs

In some embodiments, the anelloVLP has a sequence, structure, and/or function that is based on an Anellovirus (e.g., an Anellovirus as described herein, e.g., an Anellovirus comprising a nucleic acid or polypeptide comprising a sequence as shown in any one of Tables A1-A32), or fragments or portions thereof, or other substantially non-pathogenic virus, e.g., a symbiotic virus, commensal virus, native virus. In some embodiments, an Anellovirus-based anelloVLP comprises at least one element exogenous to that Anellovirus, e.g., an exogenous effector or a nucleic acid sequence encoding an exogenous effector. In some embodiments, the anelloVLP comprises a surface moiety comprising the exogenous effector. In some embodiments, an Anellovirus-based anelloVLP comprises at least one element heterologous to another element from that Anellovirus, e.g., an effector-encoding nucleic acid sequence that is heterologous to another linked nucleic acid sequence, such as a promoter element. An anelloVLP may be a delivery vehicle (e.g., a substantially non-pathogenic delivery vehicle) for a payload into a host, e.g., a human. In some embodiments, the anelloVLP is not capable of replicating in a eukaryotic cell, e.g., a mammalian cell, e.g., a human cell. In some embodiments, the anelloVLP is substantially non-pathogenic and/or substantially non-integrating in the mammalian (e.g., human) cell. In some embodiments, the anelloVLP is substantially non-immunogenic in a mammal, e.g., a human.

In an aspect, the invention includes an anelloVLP comprising a proteinaceous exterior and an effector (e.g., an exogenous effector); wherein the anelloVLP is capable of delivering the exogenous effector into a eukaryotic cell. In some embodiments, the exogenous effector is enclosed within the proteinaceous exterior. In some embodiments, the exogenous effector is comprised in a surface moiety on the surface of the anelloVLP (e.g., as described herein). In some embodiments, the proteinaceous exterior comprises one or more ORF1 molecules (e.g., an Anellovirus ORF1 protein, e.g., as described herein, or a polypeptide having at least 50%, 60%, 70%, 75%, 80%, 85%, 90%, 95%, 96%, 97%, 98%, or 99% sequence identity thereto).

In some embodiments, the anelloVLP includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anelloVLP modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

The anelloVLPs, compositions comprising anelloVLPs, methods using such anelloVLPs, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce anelloVLPs which can then be used to deliver an effector to cells (e.g., animal cells, e.g., human cells or non-human animal cells such as pig or mouse cells). In embodiments, the effector can silence expression of a factor such as an interferon. The examples further describe how anelloVLPs can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anelloVLP is introduced into a cell (e.g., a human cell). In some embodiments, the exogenous effector is delivered to the cell. In some embodiments, delivery of the exogenous effector to a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, delivery of the exogenous effector to a cell modulates (e.g., increases or decreases) a function of the cell. In some embodiments, delivery of the exogenous effector to a cell modulates (e.g., increases or decreases) the viability of the cell. In some embodiments, delivery of the exogenous effector to a cell decreases viability of a cell (e.g., a cancer cell).

In some embodiments, an anelloVLP (e.g., a synthetic anelloVLP) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anelloVLP based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anelloVLP based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

### Anelloviruses

In some embodiments, an anellovector or anelloVLP, e.g., as described herein, comprises sequences or expression products derived from an Anellovirus. In some embodiments, an anellovector or anelloVLP includes one or more sequences or expression products that are exogenous relative to the Anellovirus. In some embodiments, an anellovector or anelloVLP includes one or more sequences or expression products that are endogenous relative to the Anellovirus. In some embodiments, an anellovector or anelloVLP includes one or more sequences or expression products that are heterologous relative to one or more other sequences or expression products in the anellovector. Anelloviruses generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001) 33:121-125). There is a suggestion that these genogroups can recombine within infected humans (Rey et al, Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234).

In some embodiments, an anellovector or anelloVLP as described herein comprises one or more polypeptides (e.g., ORF1 molecules) comprising an amino acid sequence having at least about 50%, 60%, 70%, 75%, 80%, 85%, 90%, 95%, 96%, 97%, 98%, 99%, or 100% sequence identity to an Anellovirus sequence, e.g., as described herein, or a fragment thereof. In embodiments, the polypeptide comprises an amino acid sequence encoded by a nucleic acid sequence selected from a sequence as shown in any one of Tables N1-N32, or a sequence having at least 70%, 75%, 80%, 85%, 90%, 95%, 96%, 97%, 98%, 99%, or 100% sequence identity thereto. In embodiments, the polypeptide comprises a sequence as shown in any one of Tables A1-A32, or a sequence having at least 70%, 75%, 80%, 85%, 90%, 95%, 96%, 97%, 98%, 99%, or 100% sequence identity thereto.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an Anelloviridae family vector (e.g., anellovector) as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the Anelloviridae family vector (e.g., anellovector) comprises a nucleic acid sequence selected from a sequence as shown in any of Tables N1-N32, or a sequence having at least 70%, 75%, 80%, 85%, 90%, 95%, 96%, 97%, 98%, 99%, or 100% sequence identity thereto. In embodiments, the Anelloviridae family vector (e.g., anellovector) comprises a polypeptide comprising a sequence as shown in Table A1-A32, or a sequence having at least 70%, 75%, 80%, 85%, 90%, 95%, 96%, 97%, 98%, 99%, or 100% sequence identity thereto.

In some embodiments, an Anelloviridae family vector (e.g., anellovector) as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviridae family viruses (e.g., Anellovirus) described herein (e.g., an Anelloviridae family virus (e.g., Anellovirus) sequence as annotated, or as encoded by a sequence listed, in any of Tables N1-N32. In some embodiments, the nucleic acid molecule comprises a sequence encoding a capsid protein, e.g., an ORF1, ORF1/1, ORF1/2, ORF2, ORF2/2, ORF2/3, or ORF2t/3 sequence of any of the Anelloviruses described herein (e.g., an Anelloviridae family virus (e.g., Anellovirus) sequence as annotated, or as encoded by a sequence listed, in any of Tables N1-N32). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 or ORF2 protein (e.g., an ORF1 or ORF2 amino acid sequence as shown in Table A1-A32, or an ORF1 or ORF2 amino acid sequence encoded by a nucleic acid sequence as shown in any of Tables N1-N32). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 protein (e.g., an ORF1 amino acid sequence as shown in Table A1-A32, or an ORF1 amino acid sequence encoded by a nucleic acid sequence as shown in any of Tables N1-N32).

### Nucleic Acid Sequences

In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF1 nucleotide sequence of any of Tables N1-N32. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF2 nucleotide sequence of any of Tables N1-N32. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) ORF3 nucleotide sequence of any of Tables N1-N32. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) GC-rich region nucleotide sequence of any of Tables N1-N32. In some embodiments, the nucleic acid molecule comprises a nucleic acid sequence having at least about 70%, 75%, 80%, 85%, 90%, 95%, 96%, 97%, 98%, 99%, or 100% sequence identity to the Anelloviridae family virus (e.g., Anellovirus) 5′ UTR conserved domain nucleotide sequence of any of Tables N1-N32.

It is understood that Tables N1-N32 herein provide the positive strand sequence corresponding to a particular Anellovirus. However, as described herein, a genetic element is typically a negative strand (e.g., comprising the reverse complement of a nucleic acid sequence as listed in any of Tables N1-N32, or a portion thereof). Consequently, a 5′ UTR conserved domain of a genetic element as described herein may comprise the reverse complement of a sequence annotated as a 5′ UTR conserved domain (e.g., in any of Tables N1-N32). Consequently, a GC-rich region of a genetic element as described herein may comprise the reverse complement of a sequence annotated as a GC-rich region (e.g., in any of Tables N1-N32).

In some embodiments, the first Anellovirus genomic sequence is the genomic sequence of an Alphatorquevirus. In some embodiments, the second Anellovirus genomic sequence is the genomic sequence of an Alphatorquevirus. In some embodiments, the first Anellovirus genomic sequence is the genomic sequence of a Betatorquevirus. In some embodiments, the second Anellovirus genomic sequence is the genomic sequence of a Betatorquevirus. In some embodiments, the first Anellovirus genomic sequence is the genomic sequence of a Gammatorquevirus. In some embodiments, the second Anellovirus genomic sequence is the genomic sequence of a Gammatorquevirus. In some embodiments, the first Anellovirus genomic sequence is a Ring1, Ring3.1, Ring4, Ring5.2, Ring 6.0, Ring7, or Ring20 Anellovirus. In some embodiments, the second Anellovirus genomic sequence is a Ring1, Ring3.1, Ring4, Ring5.2, Ring 6.0, Ring7, or Ring20 Anellovirus. The Ring1 Anellovirus genomic sequence is disclosed as SEQ ID NO: 16 of International Application PCT/US2021/037076, and the corresponding amino acid sequences are disclosed in Table A2 of the same International Application, and the sequences are herein incorporated by reference in their entireties. The Ring3.1 Anellovirus genomic sequence is disclosed as SEQ ID NO: 878 of International Application PCT/US2022/015499, and the corresponding amino acid sequences are disclosed in Table B4 of the same International Application, and the sequences are herein incorporated by reference in their entireties. The Ring4 Anellovirus genomic sequence is disclosed as SEQ ID NO: 886 of International Application PCT/US2021/037076, and the corresponding amino acid sequences are disclosed in Table C2 of the same International Application, and the sequences are herein incorporated by reference in their entireties. The Ring5.2 Anellovirus genomic sequence is disclosed as SEQ ID NO: 894 of International Application PCT/US2022/015499, and the corresponding amino acid sequences are disclosed in Table D2 of the same International Application, and the sequences are herein incorporated by reference in their entireties. The Ring6.0 Anellovirus genomic sequence is disclosed as SEQ ID NO: 903 of International Application PCT/US2019/065995, and the corresponding amino acid sequences are disclosed in Table C4 of the same Internation Application, and the sequences are herein incorporated by reference in their entireties. The Ring7 Anellovirus genomic sequence is disclosed as SEQ ID NO: 911 of International Application PCT/US2019/065995, and the corresponding amino acid sequences are disclosed in Table C5 of the same International Application, and the sequences are herein incorporated by reference in their entireties. The Ring20 Anellovirus genomic sequence is disclosed as SEQ ID NO: 1014 of International Application PCT/US2022/015499, and the corresponding amino acid sequences are disclosed in Table F6 of the same International Application, and the sequences are herein incorporated by reference in their entireties.

### Amino Acid Sequences Encoded by Nucleic Acid Sequences

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Tables A1-A32. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Tables A1-A32. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Tables A1-A32. In some embodiments, the nucleic acid is a genetic element construct or a construct for providing the polypeptide (e.g., an ORF1 molecule and/or an ORF2 molecule) in trans.

### Proteins Comprising Amino Acid Sequences

In some embodiments, the Anelloviridae family vector described herein comprises an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described herein). In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Table A1-A32. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Table A1-A32. In embodiments, the Anelloviridae family vector described herein comprises a protein having an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Table A1 or A3. In some embodiments, an ORF1 molecule (e.g., comprised in the Anelloviridae family vector) comprises a polypeptide encoded by the Anelloviridae family virus (e.g., Anellovirus) ORF1 nucleic acid sequence of any of Tables N1-N32. In some embodiments, the ORF1 molecule (e.g., comprised in the Anelloviridae family vector) comprises an Anelloviridae family virus (e.g., Anellovirus) ORF1 protein of Table A1-A32 or a splice variant or post-translationally processed (e.g., proteolytically processed) variant thereof. In some embodiments, an ORF2 molecule (e.g., comprised in the Anelloviridae family vector) comprises a polypeptide encoded by the Anelloviridae family virus (e.g., Anellovirus) ORF2 nucleic acid sequence of any of Tables N1-N32. In some embodiments, the ORF2 molecule (e.g., comprised in the Anelloviridae family vector) comprises an Anelloviridae family virus (e.g., Anellovirus) ORF2 protein of Table A1-A32 or a splice variant or post-translationally processed (e.g., proteolytically processed) variant thereof. In some embodiments, an ORF3 molecule (e.g., comprised in the Anelloviridae family vector) comprises a polypeptide encoded by the Anelloviridae family virus (e.g., Anellovirus) ORF3 nucleic acid sequence of any of Tables N1-N32. In some embodiments, the ORF3 molecule (e.g., comprised in the Anelloviridae family vector) comprises an Anelloviridae family virus (e.g., Anellovirus) ORF3 protein of Table A1 or Table A3 or a splice variant or post-translationally processed (e.g., proteolytically processed) variant thereof.

### Polypeptides Comprising Amino Acid Sequences

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF1 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF1 amino acid sequence of Table A1-A32.

In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anelloviridae family virus (e.g., Anellovirus) ORF1 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anelloviridae family virus (e.g., Anellovirus) ORF1 nucleic acid as listed in Table N1-N32.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF2 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF2 amino acid sequence of Table A1-A32.

In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anelloviridae family virus (e.g., Anellovirus) ORF2 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anelloviridae family virus (e.g., Anellovirus) ORF2 nucleic acid as listed in Table N1-N32.

In some embodiments, the polypeptide described herein comprises an amino acid sequence having at least about 70%, 75%, 80%, 85%, 90%, 95%, 96%, 97%, 98%, 99%, or 100% sequence identity to an Anelloviridae family virus (e.g., Anellovirus) ORF3 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anelloviridae family virus (e.g., Anellovirus) ORF3 amino acid sequence of Table A1-A32.

In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anelloviridae family virus (e.g., Anellovirus) ORF3 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anelloviridae family virus (e.g., Anellovirus) ORF3 nucleic acid as listed in Table N1-N32.

In some embodiments, the polypeptide comprises an amino acid sequence (e.g., an ORF1, ORF1/1, ORF1/2, ORF2, ORF2/2, ORF2/3, or ORF2t/3 sequence) as shown in Table A1-A32, or a sequence having at least 70%, 75%, 80%, 85%, 90%, 95%, 96%, 97%, 98%, 99%, or 100% sequence identity thereto.

TABLE N1
Novel Anellovirus nucleic acid sequence (Alphatorquevirus)

| Name | TTV-RTx1 |
|---|---|
| Genus/Clade | Alphatorquevirus, Clade 6 |
| Accession Number | SRR2167793 |
| Full Sequence: 3648 bp | |
| (SEQ ID NO: 830) | |
| Annotations: | |
| Putative Domain | Base range |
| TATA Box | 77 - 81 |
| Initiator Element | 95-110 |
| Transcriptional Start Site | 105 |
| 5′ UTR Conserved Domain | 165 - 235 |
| ORF2 | 335 - 703 |
| ORF2/2 | 335 - 699 ; 2326 - 2759 |
| ORF2/3 | 335 - 699 ; 2552 - 2957 |
| ORF2t/3 | 335-465 ; 2552 - 2957 |
| ORF1 | 574 - 2775 |
| ORF1/1 | 574 - 699 ; 2326 - 2775 |
| ORF1/2 | 574 - 699 ; 2552 - 2759 |
| Three open-reading frame region | 2535 - 2746 |
| Poly(A) Signal | 2953 - 2958 |
| GC-rich region** | 3620 - 3648 |

TABLE A1
Novel Anellovirus amino acid sequences (Alphatorquevirus, Clade 6)
TTV-RTx1 (Alphatorquevirus Clade 6)

| | |
|---|---|
| ORF2 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEE (SEQ ID NO: 831) |
| ORF2/2 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEEQSKTLVKTKVPDPIDSLETYKLRTRQPWDLN |
| | MCSTRGTGDGASLASELSDACSKNLSTMMSILKNQKDLEYFLQQKQSPETK |
| | SSKKARFQRKKSRYSPQKRSRKRRYSDSSSDSSSDSCASGSSSNSSNNNSSRR |
| | KRACT (SEQ ID NO: 832) |
| ORF2/3 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEENRVPKPRARRKLAFRGRKVATLHRRDPERG |
| | DTATVQATAQATAAPRAAAQTPPTTTPQDASGPAPKPPFIFPAINKVYLFPD |
| | RAPKPKPTSGDWETEYAACSAFDRPARTNLSSPPYYPGVPTPWQVKFSLKF |
| | Q (SEQ ID NO: 833) |
| ORF2t/3 | MSWSRPVHNATGIERNWWESCLRSHASSCGCGNFVNHINVLANRNRVPKP |
| | RARRKLAFRGRKVATLHRRDPERGDTATVQATAQATAAPRAAAQTPPTTTP |
| | QDASGPAPKPPFIFPAINKVYLFPDRAPKPKPTSGDWETEYAACSAFDRPART |
| | NLSSPPYYPGVPTPWQVKFSLKFQ (SEQ ID NO: 834) |
| ORF1 | MAWGYWWRRRFWRPRRRWRTWRRRRRLRPRRSRRTFRRTRRRVRRRRW |
| | GRRARRRGWRRRTYIRARRRRRRKRLVLTQWHPAVRRKCKITGYMPIVYC |
| | GHGRASFNYAWHSDDCIKQPLPFGGSLSTVSFNLKVLFDENQRGLNKWSYP |
| | NDQLDLARYKGCRLTFYRKKNTDYIAQYDISEPYQLDKYSCANYHPSKMM |
| | FAKNKILIPSYDTKPRGRQRVRVRIGPPKLFTDKWYSQSDLCKVNLVSLAVS |
| | AASFLHPFGSPQTANFCATFQVLQPFYYQAIGISSTKHSEVIDILYKKNTYWQ |
| | SNITSWFLTNVKNPKNMSTKMFEDINVKSNKDSNYDWFPFTPYTTENYSKIQ |
| | NAAQEYWKYLTSDHPQATNSNEGLVQPWTNATIKQYEYHLGMFSPIFIGPT |
| | RAKTKFKTAYFDCTYNPLLDKGMGNRIWYQYATKADTQISKTGCYCMLED |
| | IPIYAAFYGYVDFIEMEIGKGQDIKENGLICCICRYTDPPMYNEQHPDMGFVF |
| | YNTNFGNGKWIDGRGDIPTYWMQRWRPVVLFQTDVIRDLVETGPFSYKDD |
| | LANTSLTMKYEFYFTWGGNQAYHQTIKNPCKDEGTGPHRQPRDVQVTDPT |
| | TVGPEYVFHAWDWRRGFLSERALRRMFEKPLNYDEYSKKPKRPRIFPPTET |
| | ESRNQELEESSLSEEEKSLLSTEEIQKEEIQRQFKRQLKRQLRLGQQLKLLQQ |
| | QLLKTQAGLHLNPLSYFPQ (SEQ ID NO: 835) |
| ORF1/1 | MAWGYWWRRRFWRPRRRWRTWRRRRRLRPRRSRRTFRRTRRRTIKNPCK |
| | DEGTGPHRQPRDVQVTDPTTVGPEYVFHAWDWRRGFLSERALRRMFEKPL |
| | NYDEYSKKPKRPRIFPPTETESRNQELEESSLSEEEKSLLSTEEIQKEEIQRQFK |
| | RQLKRQLRLGQQLKLLQQQLLKTQAGLHLNPLSYFPQ (SEQ ID NO: 836) |
| ORF1/2 | MAWGYWWRRRFWRPRRRWRTWRRRRRLRPRRSRRTFRRTRRRKQSPETK |
| | SSKKARFQRKKSRYSPQKRSRKRRYSDSSSDSSSDSCASGSSSNSSNNNSSRR |
| | KRACT (SEQ ID NO: 837) |

TABLE N2
Novel Anellovirus nucleic acid sequence (Alphatorquevirus)

| Name | TTV-RTx2 |
|---|---|
| Genus/Clade | Alphatorquevirus, Clade 6 |
| Accession Number | SRR3479021 |
| Full Sequence: 3704 bp | |
| (SEQ ID NO: 838) | |
| Annotations: | |
| Putative Domain | Base range |
| TATA Box | 87 - 91 |
| Initiator Element | 105-120 |
| Transcriptional Start Site | 115 |
| 5′ UTR Conserved Domain | 175 - 245 |
| ORF2 | 342 - 728 |
| ORF2/2 | 342 - 724 ; 2414 - 2849 |
| ORF2/3 | 342 - 724 ; 2643 - 3057 |
| ORF1 | 599 - 2887 |
| ORF1/1 | 599 - 724 ; 2414 - 2887 |
| ORF1/2 | 599 - 724 ; 2643 - 2849 |
| Three open-reading frame region | 2626 - 2846 |
| Poly(A) Signal | 3052 - 3058 |

TABLE A2
Novel Anellovirus amino acid sequences (Alphatorquevirus, Clade 6)
TTV-RTx2 (Alphatorquevirus Clade 6)

| | |
|---|---|
| ORF2 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDE (SEQ ID NO: 839) |
| ORF2/2 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDERSETHAPRTDIPVPVDSLETYKWLTR |
| | SKWAPGSCSTPGTGAEATLAQQLSKELESNRSIMKLIRTAQRDLESFLPQKE |
| | TSSPEVEKKTHFQRKKVPISRSKRGRNRKPRRYSSTSSDTSESSENSESDSEPC |
| | SKASKRRRRVST (SEQ ID NO: 840) |
| ORF2/3 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDERRPARPKSRRRLIFRGRKSPYLVRRG |
| | AGTESPGGTAAPPPTPQKAARTPKATPSPVPKPPKDAGGSPRKSIIIQPACNQ |
| | VLMFPEMGPKPAPTAQDWQCEYETCKHWDRPPRKFLTDPPFYPWAPTTYN |
| | VSFKLNFK (SEQ ID NO: 841) |
| ORF1 | MAYWGWWRRRRWPRRRWRRYRYRRRLPRRRPRRAVRRPRRRRVRRRRG |
| | RGWYRGRRYSRRRYRRRYVRRKRKTLVWRQWQPQNIRKCRIRGIIPILICGH |
| | GRGARNYALHSDDITPQNTPFGGGLSTTSWSLKVLYDQHTRGLNRWSASNE |
| | SLDLARYNGCSFTFYRDKKTDFIVTYDTSAPYKLDKYSSPSYHPGSMMLMT |
| | KHKILIPSFDTKPKGPAKIRVRIKPPKMFLDKWYTQDDLCSVNLVSLAVSAA |
| | SFTHPFCPPLTDTPCVTLQVLKDFYYTTIGYSSNADKVESVFTNTLYKHCCY |
| | YQSFLTTQFIAKITRTPDGQPVATFSPPTSFPGTTVTKSSIESFNQWVTSTGTS |
| | GWLTNANQHFHFCNYKPDATKLKWLRQYYFDWETYKLADVKPDGLTPSV |
| | NWYEYRIGLFSPIFLSPFRSSSLDFPRAYQDVNYNPLVDKGVGNIIWFQYNTK |
| | PDTQLSVPSCKCVIEDKPLWAAFYGYSDFVQQEIGDYTDAEAVGFVCVICPY |
| | TKPPLKNPDNPMQGFIFYDSLFGNGKWIDGTGHVPLYWQSRWRPEMLFQE |
| | NTMRDITLSGPFSYKDDYKNCVLTCKYKFNFRFGGNLLHEQTIRNPCPTDGH |
| | PSTGRQPRDVQVVDPIKVGPRFVFHSWDWRRGYLSPAALKRIGEQPLDYEA |
| | YSYRPKRPRIFPPTEGDQLARSREEDSFSEEESPHISFEEGQEPKAQAVQQHLL |
| | RHLRKQRELRKRLRALFQSLQKTQAGLHVNPLLFNQPAIRF (SEQ ID NO: |
| | 842) |
| ORF1/1 | MAYWGWWRRRRWPRRRWRRYRYRRRLPRRRPRRAVRRPRRRRTIRNPCP |
| | TDGHPSTGRQPRDVQVVDPIKVGPRFVFHSWDWRRGYLSPAALKRIGEQPL |
| | DYEAYSYRPKRPRIFPPTEGDQLARSREEDSFSEEESPHISFEEGQEPKAQAV |
| | QQHLLRHLRKQRELRKRLRALFQSLQKTQAGLHVNPLLFNQPAIRF (SEQ ID |
| | NO: 843) |
| ORF1/2 | MAYWGWWRRRRWPRRRWRRYRYRRRLPRRRPRRAVRRPRRRRKETSSPE |
| | VEKKTHFQRKKVPISRSKRGRNRKPRRYSSTSSDTSESSENSESDSEPCSKAS |
| | KRRRRVST (SEQ ID NO: 844) |

TABLE N3
Novel Anellovirus nucleic acid sequence (Alphatorquevirus)

| Name | TTV-RTx3 |
|---|---|
| Genus/Clade | Alphatorquevirus, Clade 4 |
| Accession Number | SRR3479781 |
| Full Sequence: 3653 bp | |
| (SEQ ID NO: 845) | |
| Annotations: | |
| Putative Domain | Base range |
| TATA Box | 50 - 55 |
| Initiator Element | 68-83 |
| Transcriptional Start Site | 78 |
| 5′ UTR Conserved Domain | 138 - 208 |
| ORF2 | 305 - 691 |
| ORF2/2 | 305 - 687 ; 2422 - 2878 |
| ORF2/3 | 305 - 687 ; 2564 - 3317 |
| ORF2t/3 | 305 - 360 ; 2564 - 3317 |
| ORF1 | 556 - 2904 |
| ORF1/1 | 556 - 687 ; 2422 - 2904 |
| ORF1/2 | 556 - 687 ; 2564 - 2878 |
| Three open-reading frame region | 2626 - 2846 |
| Poly(A) Signal | 3316 - 3319 |

TABLE A3
Novel Anellovirus amino acid sequences (Alphatorquevirus, Clade 4)
TTV-RTx3 (Alphatorquevirus Clade 4)

| | |
|---|---|
| ORF2 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQ (SEQ ID NO: 846) |
| ORF2/2 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQSSETRAKTPKYPDPVDSLAKYKSLT |
| | RNSLRRRGSSTRSTRDEECLLQELSNVCSSNQYLASMLLHHSGSRSSFPPQSS |
| | SEREKMQKAAQVHHPRDRDSGRKRPTRRKRSPRRGRRRRRGSSSSESSESSE |
| | SSTSNSSMSQYNSPKPKRTST (SEQ ID NO: 847) |
| ORF2/3 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQSYQTSAQATNTWRVCSYTTQGPAP |
| | LSLLRVPARGRRCRKRLRFTTQETATLAGRGQPDANGVLGGAGGDDEGAP |
| | RAKAQRAASPQPPTPACRSTTRQNPSEPPHKPPIILPALNKVYLFPPDKPTPIQ |
| | XXXXXXNTEFEACQAFDRPPRKYLSDTPTYPWLPVPNPEIKVSFKLGFKSYK |
| | AVGVSLVGVYLLRSLSTPSVSEECDPSPWCNALGGRALRLRLRAAPRTPAR |
| | ADALARVRPLRARGGREFC (SEQ ID NO: 848) |
| ORF2t/3 | MSSTWRVPRGDVSARELCWSYQTSAQATNTWRVCSYTTQGPAPLSLLRVP |
| | ARGRRCRKRLRFTTQETATLAGRGQPDANGVLGGAGGDDEGAPRAKAQR |
| | AASPQPPTPACRSTTRQNPSEPPHKPPIILPALNKVYLFPPDKPTPIQXXXXXX |
| | NTEFEACQAFDRPPRKYLSDTPTYPWLPVPNPEIKVSFKLGFKSYKAVGVSL |
| | VGVYLLRSLSTPSVSEECDPSPWCNALGGRALRLRLRAAPRTPARADALAR |
| | VRPLRARGGREFC (SEQ ID NO: 849) |
| ORF1 | MAWWGWRRRWWRPRRRWRWRRPRRRRRVPARRPRRAVRRYRTRTVRR |
| | RRRGWRRGYRRRYRLRRYRRRGRRRKKIVLTQWNPQTVRKCFIRGLMPVL |
| | WAGMGTGGHNYAVRSDDFVVDRGFGGSFATETFSLRVLFDQYQRGFNRW |
| | SHTNEDLDLARYTGCKWTFYRHQDTDFIVYFTNNPPMKTNQHTAPLTTPG |
| | MLMRSKYKILVPSFKTRPKGRKTVSVRVRPPKLFQDKWYTQQDLCPVPLVQ |
| | LNVTAADFTHPFGSPLTDTPCIRFQVLGNLYNKCLNIDLPQFDEDGEILTSTP |
| | YNRENKEDLKKLYKTLFVDEHAGNYWQTFLTNTMVKSHIDANQAKTYDQ |
| | EKTAAEQGKDPFPTNPPKDQFTTWNKKLVDPRDSNFLFATYHPKNIKKAIKT |
| | MRDNNFALTTGKNDIYGDYTAAYTRNTHMLDYYLGFYSPIFLSSGRSNTEF |
| | WTAYRDIVYNPLLDKGTGNMIWFQYHTKTDNIYKKPECHWEILDMPLWAL |
| | CNGYVEYLESQIKYGDILVEGKVLIRCPYTKPALVDPNNSLAGYVVENTTFG |
| | QGKWIDGKGYIPLHERSKWYVMLRYQTDVLHDIVTCGPWQYRDDNKNSQ |
| | LIAKYRFKFYWGGNMVHSQVIRNPCKDTQVSGPRRQPREVQVVDPQLITPP |
| | WVLHSFDQRRGMFTAGAIKRLLKQPIPGEYAPTPLRVPLLFPSSEFQREGED |
| | AESGSGSPPKRPRLWQEEANQTQTESSEGPAETTRELLERKLREQRVLNLQL |
| | QHVAVQLAKTQANLHINPLLYSQP (SEQ ID NO: 850) |
| ORF1/1 | MAWWGWRRRWWRPRRRWRWRRPRRRRRVPARRPRRAVRRYRTRTVIRN |
| | PCKDTQVSGPRRQPREVQVVDPQLITPPWVLHSFDQRRGMFTAGAIKRLLK |
| | QPIPGEYAPTPLRVPLLFPSSEFQREGEDAESGSGSPPKRPRLWQEEANQTQT |
| | ESSEGPAETTRELLERKLREQRVLNLQLQHVAVQLAKTQANLHINPLLYSQP |
| | (SEQ ID NO: 852) |
| ORF1/2 | MAWWGWRRRWWRPRRRWRWRRPRRRRRVPARRPRRAVRRYRTRTELSN |
| | VCSSNQYLASMLLHHSGSRSSFPPQSSSEREKMQKAAQVHHPRDRDSGRKR |
| | PTRRKRSPRRGRRRRRGSSSSESSESSESSTSNSSMSQYNSPKPKRTST (SEQ |
| | ID NO: 853) |

TABLE N4
Novel Anellovirus nucleic acid sequence (Alphatorquevirus)

| Name | TTV-RTx4 |
|---|---|
| Genus/Clade | Alphatorquevirus, Clade 4 |
| Accession Number | SRR3481579 |
| Full Sequence: 3742 bp | |
| (SEQ ID NO: 854) | |
| Annotations: | |
| Putative Domain | Base range |
| TATA Box | 86 - 90 |
| Initiator Element | 104 - 119 |
| Transcriptional Start Site | 114 |
| 5′ UTR Conserved Domain | 174 - 244 |
| ORF2 | 353 - 715 |
| ORF2/2 | 353 - 711 ; 2362 - 2863 |
| ORF2/3 | 353 - 711 ; 2555 - 3065 |
| ORF2t/3 | 353 - 432; 2555 - 3065 |
| ORF1 | 589 - 2889 |
| ORF1/1 | 589 - 711 ; 2362 - 2889 |
| ORF1/2 | 589 - 711 ; 2555 - 2863 |
| Three open-reading frame region | 2555 - 2863 |
| Poly(A) Signal | 3062 - 3066 |
| GC-rich region, or a portion thereof* | 3720 - 3742 |

TABLE A4
Novel Anellovirus amino acid sequences (Alphatorquevirus, Clade 4)
TTV-RTx4 (Alphatorquevirus Clade 4)

| ORF2 | MWTPPRNDQQYLNWQWYSSILSSHAAMCGCPDVVAHFNHLASVLRAPQN |
|---|---|
| | PPPPGPQRNLPLRRLPALPAAPEAPGDRAPWPMAGGAGGEDGGAGGDADH |
| | GGAAGGPEDADLLDAVAAAET (SEQ ID NO: 855) |
| ORF2/2 | MWTPPRNDQQYLNWQWYSSILSSHAAMCGCPDVVAHFNHLASVLRAPQN |
| | PPPPGPQRNLPLRRLPALPAAPEAPGDRAPWPMAGGAGGEDGGAGGDADH |
| | GGAAGGPEDADLLDAVAAAETLLEIPAKTPTPRSIESLEAYKSLTRNTTHRN |
| | SHSIRGTSDVASLARKLLRECNNNQQLLTFFQQAARDPGGTPRCTTPAKKGS |
| | KKKAYFSPQSSSSDESPRGKTRSRRKAGRKAQRKRRRPSPSSSSSSCSNSESW |
| | ESNSYSCSTKSKKSNKIKISTLPCYQGGGI (SEQ ID NO: 856) |
| ORF2/3 | MWTPPRNDQQYLNWQWYSSILSSHAAMCGCPDVVAHFNHLASVLRAPQN |
| | PPPPGPQRNLPLRRLPALPAAPEAPGDRAPWPMAGGAGGEDGGAGGDADH |
| | GGAAGGPEDADLLDAVAAAETPQETQEGHRGVPLQPRRGAKRKLTFPPSQ |
| | APQTSPPVGRLAAGGKRVAKLRGRDADRLPAAQAAAAATANPGSQTHTPV |
| | QPSPKNPTKSRYQPYLVTKGGGSSILISNSTINMFGDPKPYNPSSNDWKEEYE |
| | ACRIWDRPPRGNLRDTPFYPWAPKENQYRVNFKLGFQ (SEQ ID NO: 857) |
| ORF2t/3 | MWTPPRNDQQYLNWQWYSSILSSHAAMPQETQEGHRGVPLQPRRGAKRK |
| | LTFPPSQAPQTSPPVGRLAAGGKRVAKLRGRDADRLPAAQAAAAATANPGS |
| | QTHTPVQPSPKNPTKSRYQPYLVTKGGGSSILISNSTINMFGDPKPYNPSSND |
| | WKEEYEACRIWDRPPRGNLRDTPFYPWAPKENQYRVNFKLGFQ (SEQ ID |
| | NO: 858) |
| ORF1 | MAYGWWRRRRRRWRRWRRRPWRRRWRTRRRRPVRRRGRRRNVRRRRR |
| | GGRWRRRYRRWKRKGRRRKKAKIIIRQWQPNYRRRCNIVGYIPVLICGENT |
| | VSRNYATHSDDTNYPGPFGGGMTTDKFTLRILYDEYKRFMNYWTASNEDL |
| | DLCRYLGVNLYFFRHPEVDFIIKINTMPPFLDTELTAPSIHPGMLALDKRARW |
| | IPSLKSRPGKKHYIKIRVGAPKMFTDKWYPQTDLCDMVLLTVYATAADMQ |
| | YPFGSPLTDSVVVNFQVLQSMYDETISILPDQKEKRITLLTSIAFYNTTQTIAQ |
| | LKPFIDAGNMTSTTTATTWGSYINTTKFNTAATTTYTYPGSTTTTVTMLTCN |
| | DSWYRGTVYNDQIKNLPKEAAQLYLKATKTLLGNTFTNDDHTLEYHGGLY |
| | SSIWLSPGRSYFETPGAYTDIKYNPFTDRGEGNMLWIDWLSKKNMNYDKLQ |
| | SKCLISDLPLWAAAYGYLEFCAKSTGDQNIHMNARLLIRSPFTDPQLLVHTN |
| | PTKGFVPYSLNFGNGKMPGGSSNVPIRMRAKWYPTLFHQQEVLEALAQSGP |
| | FAYHSDIKKVSLGMKYRFKWIWGGNPVRQQVVRNPCKDSHSSVNRVPRSL |
| | QIVDPKYNSPELTFHTWDFRRGLFGQKAIERMQQQPTTTDIFSAGRKRPRRD |
| | TEVYHSSQEGEQKESLLFPPVKLLRRVPPWEDSQQEESGSQSSEEETQTVSQ |
| | QLKQQLQQQRILGVKLILLFNQVQKIQQNQDINPTLLPRGGDLASLFQIAP |
| | (SEQ ID NO: 859) |
| ORF1/1 | MAYGWWRRRRRRWRRWRRRPWRRRWRTRRRRPVRRRGRRRNVVRNPC |
| | KDSHSSVNRVPRSLQIVDPKYNSPELTFHTWDFRRGLFGQKAIERMQQQPTT |
| | TDIFSAGRKRPRRDTEVYHSSQEGEQKESLLFPPVKLLRRVPPWEDSQQEES |
| | GSQSSEEETQTVSQQLKQQLQQQRILGVKLILLFNQVQKIQQNQDINPTLLPR |
| | GGDLASLFQIAP (SEQ ID NO: 860) |
| ORF1/2 | MAYGWWRRRRRRWRRWRRRPWRRRWRTRRRRPVRRRGRRRNAARDPG |
| | GTPRCTTPAKKGSKKKAYFSPQSSSSDESPRGKTRSRRKAGRKAQRKRRRPS |
| | PSSSSSSCSNSESWESNSYSCSTKSKKSNKIKISTLPCYQGGGI (SEQ ID NO: |
| | 861) |

TABLE N5
Novel Anellovirus nucleic acid sequence (Alphatorquevirus)

| Name | TTV-RTx5b |
|---|---|
| Genus/Clade | Alphatorquevirus, Clade 5 |
| Accession Number | SRR3481639 |
| Full Sequence: 3553 bp | |
| (SEQ ID NO: 862) | |
| Annotations: | |
| Putative Domain | Base range |
| TATA Box | 12 - 17 |
| Initiator Element | 30 - 45 |
| Transcriptional Start Site | 40 |
| 5′ UTR Conserved Domain | 100 - 171 |
| ORF2 | 272 - 637 |
| ORF2/2 | 272 - 633 ; 2326 - 2767 |
| ORF2/3 | 272 - 633 ; 2525 - 2984 |
| ORF2t/3 | 272 - 633 ; 2525 - 2984 |
| ORF1 | 511 - 2793 |
| ORF1/1 | 511 - 711 ; 2326 - 2793 |
| ORF1/2 | 511 - 711 ; 2525 - 2767 |
| Three open-reading frame region | 2525 - 2767 |
| Poly(A) Signal | 2981 - 2985 |
| Unknown sequence | 3125-3176 |
| *Note: Modifications made to maintain | |
| reading frames: | |
| “C” inserted into ORF2 | 430 |
| “N” inserted into ORF1 | 1842 |

TABLE A5
Novel Anellovirus amino acid sequences (Alphatorquevirus, Clade 5)
TTV-RTx5b (Alphatorquevirus Clade 5)

| | |
|---|---|
| ORF2 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEE (SEQ ID NO: 863) |
| ORF2/2 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEELLKTPAQTAGYDLHPVESVAQYKSLARSQW |
| | GQSTYSTGGTGDGGSLIKKLSKECLKNQLMMESIQQAQRSLDGFPHSTTKSK |
| | KAPQVQRRQGRSPRKKKPLKKPSKKSKRRRYSSTSSSSTESSDGSESNSNSSC |
| | CSSPRRRATCT (SEQ ID NO: 864) |
| ORF2/3 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEEPKGPSMVSPTRQPRARRRLRFRGDKVAVLAR |
| | RSRSRSPPRSPRGVATAAPPPAVPRAATDRKATPTRHAAAHQDAEQPAHKP |
| | PCSWPCINKVYMFPPDKPMPIHGYHGWETEYQACKAFNRPPRNYLSDKPIY |
| | PWLPRPEPEIIVSFRFGFK (SEQ ID NO: 865) |
| ORF2t/3 | MSWRPPVHNPNGIERNLWEAFFRMHASACGCGDLVGHLTVLAGRPKGPSM |
| | VSPTRQPRARRRLRFRGDKVAVLARRSRSRSPPRSPRGVATAAPPPAVPRAA |
| | TDRKATPTRHAAAHQDAEQPAHKPPCSWPCINKVYMFPPDKPMPIHGYHG |
| | WETEYQACKAFNRPPRNYLSDKPIYPWLPRPEPEIIVSFRFGFK (SEQ ID NO: |
| | 866) |
| ORF1 | MAWWGRWRRWRWRPRRWRRRRRRRVPRRRAQRPVRRRRARRVRRRRW |
| | GRRRWRRGYRRRLRLRRRRRRKRKIVLTQWNPAKVRRCTIKGVLPMILCG |
| | AGRSGFNYGLHSDDYTVQKPLGQNPHGGGMSTVTFSLQVLYDQYQRFMN |
| | KWSYSNDQLDLARYFGCTFWFYRHPEVDFVAQFDNVPPMKMDENTAPNT |
| | HPSFLLQNKHKVKIPSFKTKPFGKKRVRVTVGPPKLFEDKWYSQHDLCKVP |
| | LVSWRLTAADFRFPFCSPQTDNPCYTFQVLHEEYYPVIGTSALENGSNYNSS |
| | AITALEKFLYEKCTHYQTFATDTRLNPQRPVSSTNANKTYTPSGSQETIVWG |
| | QSDFNLFKKHTDSNYGYCTYCPTNDLATKIKKYRDKRFDWLTNMPVTNTC |
| | HINATFARGKIKEWEYHLGWFSNIFIGNLRHNLAFRAAYIDITXTDKGEGNII |
| | WFQYLTKPTTEYIEAQAKCSITNIPLYAAFYGYEDYLQRTLGPYQDVETLGII |
| | CVKCPYTDPPLVHKSTDKKNWGYVFYDVHFGNGKTPEGLGQVHPYWMQR |
| | WRPYVQFQKDTMNKIARTGPFSYRDETPSITLTAGYKFHFNWGGDSIFPQIIK |
| | NPCPDSGVRPSSSRERRSVQVVSPLTMGPEYIFHRWDWRRGFFNQKALKRM |
| | LEKSINDGEYPTGPKVPRWFPPLDNQEQEGASGSEETRSQSSQEEAAQEALQ |
| | EVQEASLQQHLLQQYREQRRIGKQLQLVMLQLTKTQSNLHINPRVLGHA |
| | (SEQ ID NO: 867) |
| ORF1/1 | MAWWGRWRRWRWRPRRWRRRRRRRVPRRRAQRPVRRRRARRIIKNPCPD |
| | SGVRPSSSRERRSVQVVSPLTMGPEYIFHRWDWRRGFFNQKALKRMLEKSI |
| | NDGEYPTGPKVPRWFPPLDNQEQEGASGSEETRSQSSQEEAAQEALQEVQE |
| | ASLQQHLLQQYREQRRIGKQLQLVMLQLTKTQSNLHINPRVLGHA (SEQ ID |
| | NO: 868) |
| ORF1/2 | MAWWGRWRRWRWRPRRWRRRRRRRVPRRRAQRPVRRRRARRAQRSLDG |
| | FPHSTTKSKKAPQVQRRQGRSPRKKKPLKKPSKKSKRRRYSSTSSSSTESSD |
| | GSESNSNSSCCSSPRRRATCT (SEQ ID NO: 869) |

TABLE N6
Novel Anellovirus nucleic acid sequence (Alphatorquevirus)

| | |
|---|---|
| Name | TTV-RTx6 |
| Genus/Clade | Alphatorquevirus, Clade 5 |
| Accession Number | SRR3438066 |
| Full Sequence: 3896 bp | |

(SEQ ID NO: 870)
Annotations:

| Putative Domain | Base range |
|---|---|
| TATA Box | 206-210 |
| Initiator Element | 224-239 |
| Transcriptional Start Site | 234 |
| 5′ UTR Conserved Domain | 294-364 |
| ORF2 | 465-830 |
| ORF2/2 | 465-826; 2534-2975 |
| ORF2/3 | 465-826; 2721-3192 |
| ORF2t/3 | 465-595; 2721-3192 |
| ORF1 | 704-3001 |
| ORF1/1 | 704-826; 2534-3001 |
| ORF1/2 | 704-826; 2721-2975 |
| Three open-reading frame region | 2721-2975 |
| Poly(A) Signal | 3189-3193 |
| Unknown sequence | 3198-3655 |
| GC-rich region, or a portion thereof** | 3844-3895 |

TABLE A6
Novel Anellovirus amino acid sequences (Alphatorquevirus, Clade 5)
TTV-RTx6 (Alphatorquevirus Clade 5)

| | |
|---|---|
| ORF2 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEE (SEQ ID NO: 871) |
| ORF2/2 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEELLKTPAQTPGFDLQPVETVAQYKSLARSQW |
| | DPSLYSTHGTGDGGSLIKKLSKECLKNQLMMENIQQAQRCLDGFPHSTTKS |
| | KKASQIQRRQPRSPRKKKPLKKPSKKSKRRRYSSTSSSSTESSEESESNSNSSC |
| | SNSPRRRATCT (SEQ ID NO: 872) |
| ORF2/3 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEEISNRPKGASMVSPTRQPRARRRLRYRDDNLA |
| | VLARRSRSRSPPRSPRGVATAAPPPAVPRAAKNRKATPTRHAPTHQDAEQP |
| | AHKSPCPWPCINKVYMFPPEKPMPIHGYHGWETEYQACKAFDRPPRNYLSD |
| | KPIYPWLPRSQPEFKVSFKLGCQ (SEQ ID NO: 873) |
| ORF2t/3 | MSWRPPVHNPNGIQRNLWEAFFRMHAAACGCGDLVGHITVLAGRISNRPK |
| | GASMVSPTRQPRARRRLRYRDDNLAVLARRSRSRSPPRSPRGVATAAPPPA |
| | VPRAAKNRKATPTRHAPTHQDAEQPAHKSPCPWPCINKVYMFPPEKPMPIH |
| | GYHGWETEYQACKAFDRPPRNYLSDKPIYPWLPRSQPEFKVSFKLGCQ |
| | (SEQ ID NO: 874) |
| ORF1 | MAWWGRWRRWRWRPRRWRRRRRRRVPRRRAQRPVRRRRARRVRRRRW |
| | GRRRWRRGYRRRLRLRRRRRRKKIRLTQWNPAKVRRCTIKGVLPMILCGA |
| | GRSGFNYGLHSDDYTVQKPLGQNPHGGGMSTVTFSLQVLFDQYQRFMNR |
| | WSYSNDQLDLARYFGCTFYFYRHPEIDFVAQYDNVPPMKMDENTAPNTHP |
| | SFLLQNKRKIKIPSFKTKPFGRKRVKVTVGPPKLFEDKWYSQHDLCKVPLVS |
| | WRLTACDFRFPFCSPLTDNPCYTFQVLHENYYPVIGTSSLENGTNYNNTAIT |
| | TLETWLYGKCTHYQTFATDTRLNPQRPVSSSNANETYTPSGSKESIIWGQSD |
| | WANFKKNTDSNYGYCSYCPSNGTNGTVDKIKKYRDQRFRWLTEMPVPNTC |
| | HIHATFARGTIKYWEYHLGWYSNIFIGNLRHNLAFRPAYIDITYNPITDKGEG |
| | NIIWFQYLTKPTTEYIETQAKCTITNIPLYAAFYGYEDYLQRTLGPYQDVETL |
| | GIICVKCPYTDPPLVHKDKSKTNWGYVFYDAHFGNGKTPEGLGQVHPYWM |
| | QRWRPYVQFQKDTMHKISRTGPFSYRDDTPSITLTAEYKFRFNWGGDSIFPQ |
| | IIKNPCPDTGVRPSTGRDRRSVQVVSPLTMGPQFIFHSWDWRRGFFNQKTLK |
| | RMLEKPVNDGEYPTGPKVPRWFPPLDNQEQEGVSDTETTTSQSSQEEAAQE |
| | ALQEVQEASLQQHLLQQYREQRRIGKQLQLVMLQLTKTQSNLHINPRVLGH |
| | A (SEQ ID NO: 875) |
| ORF1/1 | MAWWGRWRRWRWRPRRWRRRRRRRVPRRRAQRPVRRRRARRIIKNPCPD |
| | TGVRPSTGRDRRSVQVVSPLTMGPQFIFHSWDWRRGFFNQKTLKRMLEKPV |
| | NDGEYPTGPKVPRWFPPLDNQEQEGVSDTETTTSQSSQEEAAQEALQEVQE |
| | ASLQQHLLQQYREQRRIGKQLQLVMLQLTKTQSNLHINPRVLGHA (SEQ ID |
| | NO: 876) |
| ORF1/2 | MAWWGRWRRWRWRPRRWRRRRRRRVPRRRAQRPVRRRRARRNIQQAQR |
| | CLDGFPHSTTKSKKASQIQRRQPRSPRKKKPLKKPSKKSKRRRYSSTSSSSTE |
| | SSEESESNSNSSCSNSPRRRATCT (SEQ ID NO: 877) |

TABLE N7
Exemplary Anellovirus nucleic acid
sequence (Alphatorquevirus, Clade 1)

| | |
|---|---|
| Name | TTV-CT30F |
| Genus/Clade | Alphatorquevirus, Clade 1 |
| Accession Number | AB064597.1 |
| Full Sequence: 3570 bp | |

(SEQ ID NO: 1)
Annotations:

| Putative Domain | Base range |
|---|---|
| TATA Box | 84-90 |
| Cap Site | 107-114 |
| Transcriptional Start Site | 114 |
| 5′ UTR Conserved Domain | 177-247 |
| ORF2 | 299-691 |
| ORF2/2 | 299-687; 2137-2659 |
| ORF2/3 | 299-687; 2339-2831 |
| ORF2t/3 | 299-348; 2339-2831 |
| ORF1 | 571-2613 |
| ORF1/1 | 571-687; 2137-2613 |
| ORF1/2 | 571-687; 2339-2659 |
| Three open-reading frame region | 2325-2610 |
| Poly(A) Signal | 2813-2818 |
| GC-rich region | 3415-3570 |

TABLE A7
Exemplary Anellovirus amino acid sequences (Alphatorquevirus, Clade 1)
TTV-CT30F (Alphatorquevirus Clade 1)

| ORF2 | MPWRPPVHSVQGREDQWFASFFHGHASFCGCGDAVGHLNSIAPRFPRAGPP |
|---|---|
| | RPPPGLEQPNPPQQGPAGPGGPPAILALPAPPAEPDDPQPRRGGGDGGAAAG |
| | AAGDRGDRDYDEEELDELFRAAAEDDL (SEQ ID NO: 2) |
| ORF2/2 | MPWRPPVHSVQGREDQWFASFFHGHASFCGCGDAVGHLNSIAPRFPRAGPP |
| | RPPPGLEQPNPPQQGPAGPGGPPAILALPAPPAEPDDPQPRRGGGDGGAAAG |
| | AAGDRGDRDYDEEELDELFRAAAEDDFQSTTPASREPTRFPTPISTLASYKC |
| | RTRNCSDRGQCSTSGTSDVGSLAKEVLKECQNTHRMMNLLRQVSHQSETSS |
| | TRPSEEKTQSKKNAILSSKHSRKKRPQKKKNQHPKKKPRKRSYSTSSSSRDA |
| | TSESSDEGSSSSLQTSSDSARESTGTPSSHRAPTLHTRPTFSQYW (SEQ ID NO: |
| | 3) |
| ORF2/3 | MPWRPPVHSVQGREDQWFASFFHGHASFCGCGDAVGHLNSIAPRFPRAGPP |
| | RPPPGLEQPNPPQQGPAGPGGPPAILALPAPPAEPDDPQPRRGGGDGGAAAG |
| | AAGDRGDRDYDEEELDELFRAAAEDDLSPIKAKQARLGLQRRKPRAKRML |
| | FSPQSTRGRRDPRRRRTSTPRKSPERGATPPAPAPETPPASPQTRAQARLYRH |
| | PPTPPGSPLEPRAHIEPPPYIPDLLFPNTGKKKKFSPFDWETEAQLAGIFKRPM |
| | RFYPSDTPHYPWLPPKRDIPKICNINFKIKLQE (SEQ ID NO: 4) |
| ORF2t/3 | MPWRPPVHSVQGREDQWSPIKAKQARLGLQRRKPRAKRMLFSPQSTRGRR |
| | DPRRRRTSTPRKSPERGATPPAPAPETPPASPQTRAQARLYRHPPTPPGSPLEP |
| | RAHIEPPPYIPDLLFPNTGKKKKFSPFDWETEAQLAGIFKRPMRFYPSDTPHY |
| | PWLPPKRDIPKICNINFKIKLQE (SEQ ID NO: 5) |
| ORF1 | TAWWWGRWRRRWRRRRPWRPRLRRRRARRAFPRRRRRRFVSRRWRRPY |
| | RRRRRRGRRRRRRRRRHKPTLVLRQWQPDVIRHCKITGRMPLIICGKGSTQF |
| | NYITHADDITPRGASYGGNFTNMTFSLEAIYEQFLYHRNRWSASNHDLELCR |
| | YKGTTLKLYRHPDVDYIVTYSRTGPFEISHMTYLSTHPLLMLLNKHHIVVPS |
| | LKTKPRGRKAIKVRIRPPKLMNNKWYFTRDFCNIGLFQLWATGLELRNPWL |
| | RMSTLSPCIGFNVLKNSIYTNLSNLPQHREDRLNIINNTLHPHDITGPNNKKW |
| | QYTYTKLMAPIYYSANRASTYDLLREYGLYSPYYLNPTRINLDWMTPYTHV |
| | RYNPLVDKGFGNRIYIQWCSEADVSYNRTKSKCLLQDMPLFFMCYGYIDW |
| | AIKNTGVSSLARDARICIRCPYTEPQLVGSTEDIGFVPITETFMRGDMPVLAP |
| | YIPLSWFCKWYPNIAHQKEVLEAIISCSPFMPRDQGMNGWDITIGYKMDFL |
| | WGGSPLPSQPIDDPCQQGTHPIPDPDKHPRLLQVSNPKLLGPRTVFHKWDIR |
| | RGQFSKRSIKRVSEYSSDDESLAPGLPSKRNKLDSAFRGENPEQKECYSLLK |
| | ALEEEETPEEEEPAPQEKAQKEELLHQLQLQRRHQRVLRRGLKLVFTDILRL |
| | RQGVHWNPELT (SEQ ID NO: 6) |
| ORF1/1 | TAWWWGRWRRRWRRRRPWRPRLRRRRARRAFPRRRRRRFPIDDPCQQGT |
| | HPIPDPDKHPRLLQVSNPKLLGPRTVFHKWDIRRGQFSKRSIKRVSEYSSDDE |
| | SLAPGLPSKRNKLDSAFRGENPEQKECYSLLKALEEEETPEEEEPAPQEKAQ |
| | KEELLHQLQLQRRHQRVLRRGLKLVFTDILRLRQGVHWNPELT (SEQ ID |
| | NO: 7) |
| ORF1/2 | TAWWWGRWRRRWRRRRPWRPRLRRRRARRAFPRRRRRRFVSHQSETSST |
| | RPSEEKTQSKKNAILSSKHSRKKRPQKKKNQHPKKKPRKRSYSTSSSSRDAT |
| | SESSDEGSSSSLQTSSDSARESTGTPSSHRAPTLHTRPTFSQYW (SEQ ID NO: |
| | 8) |

TABLE N8
Exemplary Anellovirus nucleic acid
sequence (Alphatorquevirus, Clade 2)

| | |
|---|---|
| Name | TTV-P13-1 |
| Genus/Clade | Alphatorquevirus, Clade 2 |
| Accession Number | KT163896.1 |
| Full Sequence: 3451 bp | |

(SEQ ID NO: 9)
Annotations:

| Putative Domain | Base range |
|---|---|
| TATA Box | 112-119 |
| Initiator Element | 128-148 |
| Transcriptional Start Site | 148 |
| 5′ UTR Conserved Domain | 204-273 |
| ORF2 | 412-912 |
| ORF2/2 | 412-908; 2490-3039 |
| ORF2/3 | 412-908; 2725-3208 |
| ORF1 | 729-2972 |
| ORF1/1 | 729-908; 2490-2972 |
| ORF1/2 | 729-908; 2725-3039 |
| Three open-reading frame region | 2699-2969 |
| Poly(A) Signal | 3220-3225 |
| GC-rich region | 3302-3541 |

TABLE A8
Exemplary Anellovirus amino acid sequences (Alphatorquevirus, Clade 2)
TTV-P13-1 (Alphatorquevirus Clade 2)

| | |
|---|---|
| ORF2 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADG (SEQ ID NO: 10) |
| ORF2/2 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADGPWKTPVRKAPSSFPEPVASLAYYKSATRPSRPPRQPG |
| | TRGTGDDPSLQRRVLKECANNNHMMNCLIRALKSQNCPFPQGPPSPVPPSPP |
| | PGGKQNRSPRQTSARRRPKQKPTKRKRRSRRRESSSSSCGSSNSCKSDSWES |
| | CSSNSSDSDRGRRSTRASYNSWAPEPVPAFPGAGPSKSAYF (SEQ ID NO: 11) |
| ORF2/3 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADGARRPRCRRRLLLVGNKTGHLARRQRDGDRSRSPPRG |
| | RDGAGGGSPAPAAVGAATPAKATAGSRVPATPPTQTGGGDPPGPRIIPGPQN |
| | PYLLFPEQAPPKVPIFDPFGQKTELELCGCFDRPPRNNPYDHPFYPWLPKEPP |
| | SYYQGYKVSFKLGFHPDKHV (SEQ ID NO: 12) |
| ORF1 | MAYWWGRRRRWRRWRRRRRPLRRRRRWRRRRRWPRRRRWRRRRRRARP |
| | ARRYRRRRGRRRVRRRRRPQKLVLTQWNPQTVRKCVIRGFLPLFFCGQGAY |
| | HRNFTDHYDDVFPKGPSGGGHGSMVFNLSFLYQEFKKHHNKWSRSNLDFD |
| | LVRYKGTVIKLYRHQDFDYIVWISRTPPFQESLLTVMTHQPSVMLQAKKCII |
| | VKSYRTHPGGKPYVTAKVRPPRLLTDKWYFQSDFCNVPLFSLQFALAELRF |
| | PICSPQTDTNCINFLVLDDIYYKFLDNKPKQSSDPNDENRIKFWHGLWSTMR |
| | YLNTTYINTLFPGTDSLVAAKDTDNSVNKYPSTATKQPYKDSQYMQNIWNT |
| | SKIHALYTWVAETNYKRLQAYYTQTYGGYQRQFFTGKQYWDYRVGMFSP |
| | AFLSPSRLNPQNPGAYTEVSYNPWTDEGTGNVVCLQYLTKETSDYKPGGGS |
| | KFCIEGVPLWAALVGYVDMCKKEGKDPGIRLNCLLLVKCPYTKPQLYDKK |
| | NPEKLFVPYSYNFGHGKMPGGDKYIPIEFKDRWYPCLLHQEEWIEDIVRSGP |
| | FVPKDMPSSVTCMMRYSSLFNWGGNIIQEQAVEDPCKKGTFVVPGTSGIARI |
| | LQVSNPAKQTPTTTWHSWDWRRSLFTETGLKRMREQQPYDELSYTGPKKP |
| | KLSLPAGPAVPGAAVASSWWETKQVTSPDVSETETEAEAHQEEETEPEEGV |
| | QLQQLWEQQLLQKRQLGVVFQQLLRLRQGAEIHPGLV (SEQ ID NO: 13) |
| ORF1/1 | MAYWWGRRRRWRRWRRRRRPLRRRRRWRRRRRWPRRRRWRRRRRRARP |
| | ARRYRRRRGRRRAVEDPCKKGTFVVPGTSGIARILQVSNPAKQTPTTTWHS |
| | WDWRRSLFTETGLKRMREQQPYDELSYTGPKKPKLSLPAGPAVPGAAVASS |
| | WWETKQVTSPDVSETETEAEAHQEEETEPEEGVQLQQLWEQQLLQKRQLG |
| | VVFQQLLRLRQGAEIHPGLV (SEQ ID NO: 14) |
| ORF1/2 | MAYWWGRRRRWRRWRRRRRPLRRRRRWRRRRRWPRRRRWRRRRRRARP |
| | ARRYRRRRGRRRGPPSPVPPSPPPGGKQNRSPRQTSARRRPKQKPTKRKRRS |
| | RRRESSSSSCGSSNSCKSDSWESCSSNSSDSDRGRRSTRASYNSWAPEPVPAF |
| | PGAGPSKSAYF (SEQ ID NO: 15) |

TABLE N9
Exemplary Anellovirus nucleic acid
sequence (Alphatorquevirus, Clade 3)

| | |
|---|---|
| Name | Ring1 |
| Genus/Clade | Alphatorquevirus, Clade 3 |
| Accession Number | AJ620231.1 |
| Full Sequence: 3753 bp | |

(SEQ ID NO: 16)
Annotations:

| Putative Domain | Base range |
|---|---|
| TATA Box | 83-88 |
| Cap Site | 104-111 |
| Transcriptional Start Site | 111 |
| 5′ UTR Conserved Domain | 170-240 |
| ORF2 | 336-719 |
| ORF2/2 | 336-715; 2363-2789 |
| ORF2/3 | 336-715; 2565-3015 |
| ORF2t/3 | 336-388; 2565-3015 |
| ORF1 | 599-2830 |
| ORF1/1 | 599-715; 2363-2830 |
| ORF1/2 | 599-715; 2565-2789 |
| Three open-reading frame region | 2551-2786 |
| Poly(A) Signal | 3011-3016 |
| GC-rich region | 3632-3753 |

TABLE A9
Exemplary Anellovirus amino acid sequences (Alphatorquevirus, Clade 3)
Ring1 (Alphatorquevirus Clade 3)

| | |
|---|---|
| ORF2 | MSFWKPPVHNVTGIQRMWYESFHRGHASFCGCGNPILHITALAETYGHPTG |
| | PRPSGPPGVDPNPHIRRARPAPAAPEPSQVDSRPALTWHGDGGSDGGAGGS |
| | GSGGPVADFADDGLDQLVAALDDEE (SEQ ID NO: 17) |
| ORF2/2 | MSFWKPPVHNVTGIQRMWYESFHRGHASFCGCGNPILHITALAETYGHPTG |
| | PRPSGPPGVDPNPHIRRARPAPAAPEPSQVDSRPALTWHGDGGSDGGAGGS |
| | GSGGPVADFADDGLDQLVAALDDEELLKTPASSPPMKYPVPVTSLEEYKSS |
| | TRGSWDRTTRSGHGTCADTHLAEQVLRECQNNKKLLTLYSQAQKSLGSTS |
| | QNKKPKKKAHIHSKENRDRGRPRKKARQKPSRKRAKRSPSNSSCSSSTKSSS |
| | SSDRESKSSSSSS (SEQ ID NO: 18) |
| ORF2/3 | MSFWKPPVHNVTGIQRMWYESFHRGHASFCGCGNPILHITALAETYGHPTG |
| | PRPSGPPGVDPNPHIRRARPAPAAPEPSQVDSRPALTWHGDGGSDGGAGGS |
| | GSGGPVADFADDGLDQLVAALDDEEPKKASGRHPKTRNPRRKLTFTPKRIE |
| | TVGDRGRKRDRSPLAREPRGPLPTAVAAAVPRAAQAQTGNQSPLRAAHKD |
| | PTRGPCKPMPTVGPRQWLFPERKPAPAPSSGDWAMEFLAAKIFDRPVRSNL |
| | KDTPYYPYVKNQYNVYFDLKFE (SEQ ID NO: 19) |
| ORF2t/3 | MSFWKPPVHNVTGIQRMWPKKASGRHPKTRNPRRKLTFTPKRIETVGDRGR |
| | KRDRSPLAREPRGPLPTAVAAAVPRAAQAQTGNQSPLRAAHKDPTRGPCKP |
| | MPTVGPRQWLFPERKPAPAPSSGDWAMEFLAAKIFDRPVRSNLKDTPYYPY |
| | VKNQYNVYFDLKFE (SEQ ID NO: 20) |
| ORF1 | MAWGWWKRRRRWWFRKRWTRGRLRRRWPRSARRRPRRRRVRRRRRWR |
| | RGRRKTRTYRRRRRFRRRGRKAKLIIKLWQPAVIKRCRIKGYIPLIISGNGTF |
| | ATNFTSHINDRIMKGPFGGGHSTMRFSLYILFEEHLRHMNFWTRSNDNLELT |
| | RYLGASVKIYRHPDQDFIVIYNRRTPLGGNIYTAPSLHPGNAILAKHKILVPS |
| | LQTRPKGRKAIRLRIAPPTLFTDKWYFQKDIADLTLFNIMAVEADLRFPFCSP |
| | QTDNTCISFQVLSSVYNNYLSINTFNNDNSDSKLKEFLNKAFPTTGTKGTSLN |
| | ALNTFRTEGCISHPQLKKPNPQINKPLESQYFAPLDALWGDPIYYNDLNENK |
| | SLNDIIEKILIKNMITYHAKLREFPNSYQGNKAFCHLTGIYSPPYLNQGRISPEI |
| | FGLYTEIIYNPYTDKGTGNKVWMDPLTKENNIYKEGQSKCLLTDMPLWTLL |
| | FGYTDWCKKDTNNWDLPLNYRLVLICPYTFPKLYNEKVKDYGYIPYSYKFG |
| | AGQMPDGSNYIPFQFRAKWYPTVLHQQQVMEDISRSGPFAPKVEKPSTQLV |
| | MKYCFNFNWGGNPIIEQIVKDPSFQPTYEIPGTGNIPRRIQVIDPRVLGPHYSF |
| | RSWDMRRHTFSRASIKRVSEQQETSDLVFSGPKKPRVDIPKQETQEESSHSL |
| | QRESRPWETEEESETEALSQESQEVPFQQQLQQQYQEQLKLRQGIKVLFEQL |
| | IRTQQGVHVNPCLR (SEQ ID NO: 21) |
| ORF1/1 | MAWGWWKRRRRWWFRKRWTRGRLRRRWPRSARRRPRRRRIVKDPSFQPT |
| | YEIPGTGNIPRRIQVIDPRVLGPHYSFRSWDMRRHTFSRASIKRVSEQQETSD |
| | LVFSGPKKPRVDIPKQETQEESSHSLQRESRPWETEEESETEALSQESQEVPF |
| | QQQLQQQYQEQLKLRQGIKVLFEQLIRTQQGVHVNPCLR (SEQ ID NO: 22) |
| ORF1/2 | MAWGWWKRRRRWWFRKRWTRGRLRRRWPRSARRRPRRRRAQKSLGSTS |
| | QNKKPKKKAHIHSKENRDRGRPRKKARQKPSRKRAKRSPSNSSCSSSTKSSS |
| | SSDRESKSSSSSS (SEQ ID NO: 23) |

TABLE N10
Exemplary Anellovirus nucleic acid
sequence (Alphatorquevirus, Clade 4)

| | |
|---|---|
| Name | TTV-HD20a |
| Genus/Clade | Alphatorquevirus, Clade 4 |
| Accession Number | FR751492.1 |
| Full Sequence: 3878 bp | |

(SEQ ID NO: 24)
Annotations:

| Putative Domain | Base range |
|---|---|
| TATA Box | 82-87 |
| Initiator Element | 95-115 |
| Transcriptional Start Site | 115 |
| 5′ UTR Conserved Domain | 170-238 |
| ORF2 | 335-721 |
| ORF2/2 | 335-717; 2446-2902 |
| ORF2/3 | 335-717; 2675-3109 |
| ORF1 | 586-2928 |
| ORF1/1 | 586-717; 2446-2928 |
| ORF1/2 | 586-717; 2675-2902 |
| Three open-reading frame region | 2640-2899 |
| Poly(A) Signal | 3106-3114 |
| GC-rich region | 3768-3878 |

TABLE A10
Exemplary Anellovirus amino acid sequences (Alphatorquevirus, Clade 4)
TTV-HD20a (Alphatorquevirus Clade 4)

| | |
|---|---|
| ORF2 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQ (SEQ ID NO: 25) |
| ORF2/2 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQSSETRAKTTKYPVPVDSLGIYKSLTR |
| | NASRRRGSSTASTSEEASLLKQLSGACSRNHYLASMLLAPSGHPSSFYPQNT |
| | SEKTALQKAPQVHRPKDPVSGQKRVRRRRSPRRRTRRRRRGSSSSESSESSE |
| | HSSSNSSTSRSNSPRPRRIST (SEQ ID NO: 26) |
| ORF2/3 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQIPARRRRCRKRLRFTGQKTPYLVRRE |
| | SDGDDLLGGEPGGDDEGAPPAKAPRAASTPVPTPALRGPTRQDPGESPRKPP |
| | VIFPAMNKVFLFPPEGPKPILGKEAWQDEYETCRVWNRPARTHHTDTPFYP |
| | WAPHKFHVSFKLGFQ (SEQ ID NO: 27) |
| ORF1 | MAWWGWRRRWWRPKRRWRWRRARRRRRVPARRPRRAFRRYRTRTVRR |
| | RRRGRRRGYRRRYRLRRYARRRFRRKKIVLTQWNPQTTRKCIIRGMMPVL |
| | WAGMGTGGRNYAVRSDDYVVNKGFGGSFATETFSLKVLYDQFQRGFNRW |
| | SHTNEDLDLARYRGCRWTFYRHKDTDFIVYFTNNPPMKTNQFSAPLTTPGM |
| | LMRSKYKVLIPSFQTRPKGRKTVTVKIRPPKLFQDKWYTQQDLCSVPLVQL |
| | NVTAADFTHPFGSPLTETPCVEFQVLGDLYNTCLNIDLPQFSELGEITSAYSK |
| | PNSNNLKELYKELFTKATSGHYWQTFITNSMVRAHIDADKAKEAQRASTTP |
| | SYNNDPFPTIPVKSEFAQWKKKFTDTRDSPFLFATYHPEAIKDTIMKMRENN |
| | FKLETGPNDKYGDYTAQYQGNTHMLDYYLGFYSPIFLSDGRSNVEFFTAYR |
| | DIVYNPFLDKAQGNMVWFQYHTKTDNKFKKPECHWEIKDMPLWALLNGY |
| | VDYLETQIQYGDLSKEGKVLIRCPYTKPALVDPRDDTAGYVVYNRNFGRGK |
| | WIDGGGYIPLHERTKWYVMLRYQTDVFHDIVTCGPWQYRDDNKNSQLVA |
| | KYRFSFIWGGNTVHSQVIRNPCKDNQVSGPRRQPRDIQVVDPQRITPPWVLH |
| | SFDQRRGLFTETALRRLLQEPLPGEYAVSTLRTPLLFLPSEYQREDGAAESAS |
| | GSPAKRPRIWSEESQTETISSEENPAETTRELLQRKLREQRALQFQLQHFAVQ |
| | LAKTQANLHVNPLLSFPQ (SEQ ID NO: 28) |
| ORF1/1 | MAWWGWRRRWWRPKRRWRWRRARRRRRVPARRPRRAFRRYRTRTVIRN |
| | PCKDNQVSGPRRQPRDIQVVDPQRITPPWVLHSFDQRRGLFTETALRRLLQE |
| | PLPGEYAVSTLRTPLLFLPSEYQREDGAAESASGSPAKRPRIWSEESQTETISS |
| | EENPAETTRELLQRKLREQRALQFQLQHFAVQLAKTQANLHVNPLLSFPQ |
| | (SEQ ID NO: 29) |
| ORF1/2 | MAWWGWRRRWWRPKRRWRWRRARRRRRVPARRPRRAFRRYRTRTNTSE |
| | KTALQKAPQVHRPKDPVSGQKRVRRRRSPRRRTRRRRRGSSSSESSESSEHS |
| | SSNSSTSRSNSPRPRRIST (SEQ ID NO: 30) |

TABLE N11
Exemplary Anellovirus nucleic acid
sequence (Alphatorquevirus, Clade 5)

| | |
|---|---|
| Name | TTV-16 (TUS01) |
| Genus/Clade | Alphatorquevirus, Clade 5 |
| Accession Number | AB017613.1 |
| Full Sequence: 3818 bp | |

(SEQ ID NO: 31)
Annotations:

| Putative Domain | Base range |
|---|---|
| TATA Box | 82-86 |
| Initiator Element | 100-115 |
| Transcriptional Start Site | 115 |
| 5′ UTR Conserved Domain | 170-240 |
| ORF2 | 331-726 |
| ORF2/2 | 331-722; 2412-2847 |
| ORF2/3 | 331-722; 2638-3058 |
| ORF2t/3 | 331-380; 2638-3058 |
| ORF1 | 588-2873 |
| ORF1/1 | 588-722; 2412-2873 |
| ORF1/2 | 588-722; 2638-2847 |
| Three open-reading frame region | 2699-2969 |
| Poly(A) Signal | 3220-3225 |
| GC-rich region | 3302-3541 |

TABLE A11
Exemplary Anellovirus amino acid sequences (Alphatorquevirus, Clade 5)
TTV-16-TUS01 (Alphatorquevirus Clade 5)

| | |
|---|---|
| ORF2 | MSFTTPTINAGIREQQWFESTLRSHHSFCGCGDPVLHFTNLATRFNYLPATSS |
| | PLDPPGPAPRGRPALRRLPALPSAPATPSRELAWPTGSEGGAGGRGAGGEGG |
| | AAVEGDYREEELDELFAALEEDANQG (SEQ ID NO: 32) |
| ORF2/2 | MSFTTPTINAGIREQQWFESTLRSHHSFCGCGDPVLHFTNLATRFNYLPATSS |
| | PLDPPGPAPRGRPALRRLPALPSAPATPSRELAWPTGSEGGAGGRGAGGEGG |
| | AAVEGDYREEELDELFAALEEDANQGSLKTPVETPESFPVPVDSLEKYKSLA |
| | RFPWDRPTSSTTSTPDAGSLVKKLLKECNNNKNLMNLLHSNLRDPNFLQQP |
| | QKSSSSKKTRLQGKENRHYSKKRKKSKSSKRRQYSSSSSETSRSSSQSSSSSN |
| | SSCSNSSKPNPICI (SEQ ID NO: 33) |
| ORF2/3 | MSFTTPTINAGIREQQWFESTLRSHHSFCGCGDPVLHFTNLATRENYLPATSS |
| | PLDPPGPAPRGRPALRRLPALPSAPATPSRELAWPTGSEGGAGGRGAGGEGG |
| | AAVEGDYREEELDELFAALEEDANQGSRRNPPARRRLDFRGRKIATTARRE |
| | RSRSPPNADSTAPAPAKHPGAARNQAAAPIPLAPTPQNPIQFAFKPTIFKPFIK |
| | YDMFGDPLPHPPTAEEWETEYQCCKAFNRPPRTNLKDTPFYPWVPKPKPQF |
| | RVSFKLGFQ (SEQ ID NO: 34) |
| ORF2t/3 | MSFTTPTINAGIREQQCSRRNPPARRRLDFRGRKIATTARRERSRSPPNADST |
| | APAPAKHPGAARNQAAAPIPLAPTPQNPIQFAFKPTIFKPFIKYDMFGDPLPH |
| | PPTAEEWETEYQCCKAFNRPPRTNLKDTPFYPWVPKPKPQFRVSFKLGFQ |
| | (SEQ ID NO: 35) |
| ORF1 | MAYWFRRWGWRPRRRWRRWRRRRRRLPRRRTRRAVRGLGRRRKPRVRR |
| | RRRTRRRTYRRGWRRRRYIRRGRRKKKLILTQWNPAIVKRCNIKGGLPIIICG |
| | EPRAAFNYGYHMEDYTPQPFPFGGGMSTVTFSLKALYDQYLKHQNRWTFS |
| | NDQLDLARYRGCKLRFYRSPVCDFIVHYNLIPPLKMNQFTSPNTHPGLLMLS |
| | KHKIIIPSFQTRPGGRRFVKIRLNPPKLFEDKWYTQQDLCKVPLVSITATAAD |
| | LRYPFCSPQTNNPCTTFQVLRKNYNTVIGTSVKDQESTQDFENWLYKTDSH |
| | YQTFATEAQLGRIPAFNPDGTKNTKQQSWQDNWSKKNSPWTGNSGTYPQT |
| | TSEMYKIPYDSNFGFPTYRAQKDYILERRQCNFNYEVNNPVSKKVWPQPST |
| | TTPTVDYYEYHCGWFSNIFIGPNRYNLQFQTAYVDTTYNPLMDKGKGNKIW |
| | FQYLSKKGTDYNEKQCYCTLEDMPLWAICFGYTDYVETQLGPNVDHETAG |
| | LIIMICPYTQPPMYDKNRPNWGYVVYDTNFGNGKMPSGSGQVPVYWQCR |
| | WRPMLWFQQQVLNDISKTGPYAYRDEYKNVQLTLYYNFIFNWGGDMYYP |
| | QVVKNPCGDSGIVPGSGRFTREVQVVSPLSMGPAYIFHYFDSRRGFFSEKAL |
| | KRMQQQQEFDESFTFKPKRPKLSTAAAEILQLEEDSTSGEGKSPLQQEEKEV |
| | EVLQTPTVQLQLQRNIQEQLAIKQQLQFLLLQLLKTQSNLHLNPQFLSPS |
| | (SEQ ID NO: 36) |
| ORF1/1 | MAYWFRRWGWRPRRRWRRWRRRRRRLPRRRTRRAVRGLGRRRKPRVVK |
| | NPCGDSGIVPGSGRFTREVQVVSPLSMGPAYIFHYFDSRRGFFSEKALKRMQ |
| | QQQEFDESFTFKPKRPKLSTAAAEILQLEEDSTSGEGKSPLQQEEKEVEVLQT |
| | PTVQLQLQRNIQEQLAIKQQLQFLLLQLLKTQSNLHLNPQFLSPS (SEQ ID |
| | NO: 37) |
| ORF1/2 | MAYWFRRWGWRPRRRWRRWRRRRRRLPRRRTRRAVRGLGRRRKPRQPQ |
| | KSSSSKKTRLQGKENRHYSKKRKKSKSSKRRQYSSSSSETSRSSSQSSSSSNS |
| | SCSNSSKPNPICI (SEQ ID NO: 38) |

TABLE N12
Exemplary Anellovirus nucleic acid
sequence (Alphatorquevirus, Clade 6)

| | |
|---|---|
| Name | TTV-TJN02 |
| Genus/Clade | Alphatorquevirus, Clade 6 |
| Accession Number | AB028669.1 |
| Full Sequence: 3794 bp | |

(SEQ ID NO: 39)
Annotations:

| Putative Domain | Base range |
|---|---|
| TATA Box | 89-90 |
| Cap Site | 107-114 |
| Transcriptional Start Site | 114 |
| 5′ UTR Conserved Domain | 174-244 |
| ORF2 | 357-731 |
| ORF2/2 | 357-727; 2381-2813 |
| ORF2/3 | 357-727; 2619-3021 |
| ORF2t/3 | 357-406; 2619-3021 |
| ORF1 | 599-2839 |
| ORF1/1 | 599-727; 2381-2839 |
| ORF1/2 | 599-727; 2619-2813 |
| Three open-reading frame region | 2596-2810 |
| Poly(A) Signal | 3017-3022 |
| GC-rich region | 3691-3794 |

TABLE A12
Exemplary Anellovirus amino acid sequences (Alphatorquevirus, Clade 6)
TTV-TJN02 (Alphatorquevirus Clade 6)

| | |
|---|---|
| ORF2 | MSWRPPVHDAPGIERNWYEACFRAHAGACGCGNFIMHLNLLAGRYGFTPG |
| | SAPPGGPPPGTPQIRRARPSPAAPEQPAALPWHGDGGDGGAAGPPDAGGDA |
| | VAGAPYGEQELADLLDAIEDDEQ (SEQ ID NO: 40) |
| ORF2/2 | MSWRPPVHDAPGIERNWYEACFRAHAGACGCGNFIMHLNLLAGRYGFTPG |
| | SAPPGGPPPGTPQIRRARPSPAAPEQPAALPWHGDGGDGGAAGPPDAGGDA |
| | VAGAPYGEQELADLLDAIEDDEQRSKTRARRTDNPPTPVDTLEEYKWRTRN |
| | KWDPAGCSTPLTGEGAILARKLSNACKKNLLTMTNILHNQKDLESFLQQNQ |
| | QRESSESPKKARIQRKKGRKPLPKSRRRRRQYSSSSDDSESNSSSSSSSNSSPE |
| | KCSKRKRVST (SEQ ID NO: 41) |
| ORF2/3 | MSWRPPVHDAPGIERNWYEACFRAHAGACGCGNFIMHLNLLAGRYGFTPG |
| | SAPPGGPPPGTPQIRRARPSPAAPEQPAALPWHGDGGDGGAAGPPDAGGDA |
| | VAGAPYGEQELADLLDAIEDDEHRGRVPRARKRLVFRGRKVASLCRRADA |
| | GGDSTPPQATTQRATAAPAAAPIPHPRNVQNASGSPPKPYVIKPAINQVYLFP |
| | ERAPKPPPSSQDWQQEYEACAAWDRPPRYNLSSPPFYPSCPSKFCVKFSLGF |
| | K (SEQ ID NO: 42) |
| ORF2t/3 | MSWRPPVHDAPGIERNCRGRVPRARKRLVFRGRKVASLCRRADAGGDSTPP |
| | QATTQRATAAPAAAPIPHPRNVQNASGSPPKPYVIKPAINQVYLFPERAPKPP |
| | PSSQDWQQEYEACAAWDRPPRYNLSSPPFYPSCPSKFCVKFSLGFK (SEQ ID |
| | NO: 43) |
| ORF1 | MAWGWWRWRRRWPARRWRRRRRRRPVRRTRARRPARRYRRRRTVRTRR |
| | RRWGRRRYRRGWRRRTYVRKGRHRKKKKRLILRQWQPATRRRCTITGYLP |
| | IVFCGHTRGNKNYALHSDDYTPQGQPFGGALSTTSFSLKVLFDQHQRGLNK |
| | WSFPNDQLDLARYRGCKFIFYRTKQTDWVGQYDISEPYKLDKYSCPNYHPG |
| | NMIKAKHKFLIPSYDTNPRGRQKIIVKIPPPDLFVDKWYTQEDLCSVNLVSLA |
| | VSAASFLHPFGSPQTDNPCYTFQVLKEFYYQAIGFSASTQAMTSVLDTLYTQ |
| | NSYWESNLTQFYVLNAKKGSDTTQPLTSNMPTREEFMAKKNTNYNWYTY |
| | KAASVKNKLHQMRQTYFEELTSKGPQTTKSEEGYSQHWTTPSTNAYEYHL |
| | GMFSAIFLAPDRPVPRFPCAYQDVTYNPLMDKGVGNHIWFQYNTKADTQLI |
| | VTGGSCKAHIQDIPLWAAFYGYSDFIESELGPFVDAETVGLVCVICPYTKPP |
| | MYNKTNPAMGYVFYDRNFGDGKWTDGRGKIEPYWQVRWRPEMLFQETV |
| | MADLVQTGPFSYKDELKNSTLVCKYKFYFTWGGNMMFQQTIKNPCKTDGQ |
| | PTDSSRHPRGIQVADPEQMGPRWVFHSFDWRRGYLSEKALKRLQEKPLDYD |
| | EYFTQPKRPRIFPPTESAEGEFREPEKGSYSEEERSQASAEEQTQEATVLLLK |
| | RRLREQQQLQQQLQFLTREMFKTQAGLHLNPMLLNQR (SEQ ID NO: 44) |
| ORF1/1 | MAWGWWRWRRRWPARRWRRRRRRRPVRRTRARRPARRYRRRRTTIKNP |
| | CKTDGQPTDSSRHPRGIQVADPEQMGPRWVFHSFDWRRGYLSEKALKRLQ |
| | EKPLDYDEYFTQPKRPRIFPPTESAEGEFREPEKGSYSEEERSQASAEEQTQE |
| | ATVLLLKRRLREQQQLQQQLQFLTREMFKTQAGLHLNPMLLNQR (SEQ ID |
| | NO: 45) |
| ORF1/2 | MAWGWWRWRRRWPARRWRRRRRRRPVRRTRARRPARRYRRRRTQRESS |
| | ESPKKARIQRKKGRKPLPKSRRRRRQYSSSSDDSESNSSSSSSSNSSPEKCSKR |
| | KRVST (SEQ ID NO: 46) |

TABLE N13
Exemplary Anellovirus nucleic acid
sequence (Alphatorquevirus, Clade 7)

| | |
|---|---|
| Name | TTV-HD16d |
| Genus/Clade | Alphatorquevirus, Clade 7 |
| Accession Number | FR751479.1 |
| Full Sequence: 3866 bp | |

(SEQ ID NO: 47)
Annotations:

| Putative Domain | Base range |
|---|---|
| TATA Box | 82-86 |
| Initiator Element | 94-115 |
| Transcriptional Start Site | 115 |
| 5′ UTR Conserved Domain | 170-240 |
| ORF2 | 357-728 |
| ORF2/2 | 357-724; 2411-2870 |
| ORF2/3 | 357-724; 2646-3081 |
| ORF1 | 599-2896 |
| ORF1/1 | 599-724; 2411-2896 |
| ORF1/2 | 599-724; 2646-2870 |
| Three open-reading frame region | 2629-2867 |
| Poly(A) Signal | 3076-3086 |
| GC-rich region | 3759-3866 |

TABLE A13
Exemplary Anellovirus amino acid sequences (Alphatorquevirus, Clade 7)
TTV-HD16d (Alphatorquevirus Clade 7)

| | |
|---|---|
| ORF2 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVE (SEQ ID NO: 48) |
| ORF2/2 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVESSRTPAKTVDWNPPTPVDSVATYKLLTH |
| | TPWAPNSRSTTGTTDMAFLAKTLSTECLNNQKMMQTILTHTKGLDIFHPQT |
| | KPPKSKKKTSVSSKQHRRTQKRAIKKSSKKRKYSDSSQSSTSNSTCSSQSGSE |
| | SESNSDTYSNRCSKLRPIST (SEQ ID NO: 49) |
| ORF2/3 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVEPSRPRARKRLQFPQNSTVELRRERSRSPP |
| | RNASTPIPARAAQATPPAARRAAANRRATPIPTPTDVQNSGQSPPKPIYIYPA |
| | VKQVFMFDPPGPKAISGAKAWEDEFLTAKVWNRPVRKYYSDTPYYPWAPK |
| | PQYSVSFKLGWK (SEQ ID NO: 50) |
| ORF1 | MAWSWWWQRWRRRRWKPRRRRWRRLRWRRPRRAVRRRRRGRRVRRRR |
| | WARRRGRRRRYATRRKRRYRGRRFKKKLVLTQWHPNTMRRCLIKGIVPLV |
| | ICGHTRWNYNYALHSKDYTEEGRYPHGGALSTTTWSLKVLYDEHLKHHDF |
| | WGYPNNQLDLARYKGAKFTFYRHKKTDFIIFFNRKPPFKLNKYSCASYHPG |
| | MLMQQRHKILLPSYETKPKGRPKITVRIKPPTLLEDKWYTQQDLCDVNLLQ |
| | LVVTAADFRHPLCSPQTNTPTTTFQVLKDIYYDTMSISEPTDSYTSVNNKSTT |
| | QTFTNYSNTLENILYTRASYWNSFHATEYLNPNIIYKNGEKLFKEHEDLITW |
| | MTQTNNTGFLTKNNTAFGNNSYRPNADKIKKARKTYWNALIGTNDLATNI |
| | GQARAERFEYHLGWYSPIFLSRHRSNMNFARAYQDVTYNPNCDRGVNNRV |
| | WVQPLTKPTTEFDEKRCKCVVQHLPLWAALYCYQDFVEEELGSSSEILNSC |
| | LLVLQCPYTFPPMYDKKLPDKGFVFYDSLFGDGKMSDGRGQVDIFWQQRW |
| | YPRLATQMQVMHDITMTGPFSYRDELVSTQLTAKYTFDFMWGGNMISTQII |
| | KNPCKDSGLEPAYPGRQRRDLQIVDPYSMGPQFSFHNWDYRHGLFGQDAID |
| | RVSKQPKDDADYPNPYKRPRYFPPTDQAAQEQEKDFSFLKTAPSNSEESDQE |
| | VLQETQVLRFQPEQHKQLHLQLAERQRIGEQLRYLLQQMFKTQANLHLNPY |
| | TFTQL (SEQ ID NO: 51) |
| ORF1/1 | MAWSWWWQRWRRRRWKPRRRRWRRLRWRRPRRAVRRRRRGRRIIKNPC |
| | KDSGLEPAYPGRQRRDLQIVDPYSMGPQFSFHNWDYRHGLFGQDAIDRVSK |
| | QPKDDADYPNPYKRPRYFPPTDQAAQEQEKDFSFLKTAPSNSEESDQEVLQE |
| | TQVLRFQPEQHKQLHLQLAERQRIGEQLRYLLQQMFKTQANLHLNPYTFTQ |
| | L (SEQ ID NO: 52) |
| ORF1/2 | MAWSWWWQRWRRRRWKPRRRRWRRLRWRRPRRAVRRRRRGRRTKPPK |
| | SKKKTSVSSKQHRRTQKRAIKKSSKKRKYSDSSQSSTSNSTCSSQSGSESESN |
| | SDTYSNRCSKLRPIST (SEQ ID NO: 53) |

TABLE N14
Exemplary Anellovirus nucleic acid sequence (Betatorquevirus)

| | |
|---|---|
| Name | Ring2 |
| Genus/Clade | Betatorquevirus |
| Accession Number | JX134045.1 |
| Full Sequence: 2979 bp | |

(SEQ ID NO: 54)
Annotations:

| Putative Domain | Base range |
|---|---|
| TATA Box | 237-243 |
| Cap Site | 260-267 |
| Transcriptional Start Site | 267 |
| 5′ UTR Conserved Domain | 323-393 |
| ORF2 | 424-723 |
| ORF2/2 | 424-719; 2274-2589 |
| ORF2/3 | 424-719; 2449-2812 |
| ORF1 | 612-2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE N14.1
Exemplary Anellovirus nucleic acid sequence (Betatorquevirus)

| | |
|---|---|
| Name | Ring2 |
| Genus/Clade | Betatorquevirus |
| Full Sequence: 2979 bp | |

(SEQ ID NO: 2506)
Annotations:

| Putative Domain | Base range |
|---|---|
| 5′ non-coding region (NCR) | 1-425 |
| TATA Box | 239-245 |
| Cap Site | 262-269 |
| 5′ UTR Conserved Domain | 326-395 |
| ORF3 | 426-2814 |
| ORF2/2 | 426-2591 |
| ORF2 | 426-725 |
| ORF1 | 614-2614 |
| ORF1/1 | 614-2614 |
| ORF1/2 | 614-2591 |
| 3′ non-coding region (NCR) | 2815-2979 |
| Poly(A) Signal | 2810-2815 |
| GC-rich region | 2870-2979 |

TABLE A14
Exemplary Anellovirus amino acid sequences (Betatorquevirus)
Ring2 (Betatorquevirus)

| | |
|---|---|
| ORF2 | MSDCFKPTCYNNKTKQTHWINNLHLTHDLICFCPTPTRHLLLALAEQQETIE |
| | VSKQEKEKITRCLITTEEDGTTTDVLDGMDEVGLDALFAEDFEEKEG (SEQ |
| | ID NO: 55) |
| ORF2/2 | MSDCFKPTCYNNKTKQTHWINNLHLTHDLICFCPTPTRHLLLALAEQQETIE |
| | VSKQEKEKITRCLITTEEDGTTTDVLDGMDEVGLDALFAEDFEEKEGFNIPY |
| | PVTSMKQLRYRVQGKPQNPSYTPSTIDTGTTQQQLCHELAKTGHLKTLFLK |
| | LQSQIDSNCSNKPSNACKSRKKRRRKKKKKYSSSSATSDSSSSCTESE (SEQ |
| | ID NO: 56) |
| ORF2/3 | MSDCFKPTCYNNKTKQTHWINNLHLTHDLICFCPTPTRHLLLALAEQQETIE |
| | VSKQEKEKITRCLITTEEDGTTTDVLDGMDEVGLDALFAEDFEEKEGARSTA |
| | TAQTSPRMPANLGRNAGEKRKRSTAAHQQPQTAAAAVQRANNIIIKGPITFN |
| | CVKKVKLFDDKPKNRRFTPEEFETELQIAKWLKRPPRSFVNDPPFYPWLPPE |
| | PVVNFKLNFTE (SEQ ID NO: 57) |
| ORF1 | MPYYYRRRRYNYRRPRWYGRGWIRRPFRRRFRRKRRVRPTYTTIPLKQWQ |
| | PPYKRTCYIKGQDCLIYYSNLRLGMNSTMYEKSIVPVHWPGGGSFSVSMLT |
| | LDALYDIHKLCRNWWTSTNQDLPLVRYKGCKITFYQSTFTDYIVRIHTELPA |
| | NSNKLTYPNTHPLMMMMSKYKHIIPSRQTRRKKKPYTKIFVKPPPQFENKW |
| | YFATDLYKIPLLQIHCTACNLQNPFVKPDKLSNNVTLWSLNTISIQNRNMSV |
| | DQGQSWPFKILGTQSFYFYFYTGANLPGDTTQIPVADLLPLTNPRINRPGQSL |
| | NEAKITDHITFTEYKNKFTNYWGNPFNKHIQEHLDMILYSLKSPEAIKNEWT |
| | TENMKWNQLNNAGTMALTPFNEPIFTQIQYNPDRDTGEDTQLYLLSNATGT |
| | GWDPPGIPELILEGFPLWLIYWGFADFQKNLKKVTNIDTNYMLVAKTKFTQ |
| | KPGTFYLVILNDTFVEGNSPYEKQPLPEDNIKWYPQVQYQLEAQNKLLQTG |
| | PFTPNIQGQLSDNISMFYKFYFKWGGSPPKAINVENPAHQIQYPIPRNEHETT |
| | SLQSPGEAPESILYSFDYRHGNYTTTALSRISQDWALKDTVSKITEPDRQQLL |
| | KQALECLQISEETQEKKEKEVQQLISNLRQQQQLYRERIISLLKDQ (SEQ ID |
| | NO: 58) |
| ORF1/1 | MPYYYRRRRYNYRRPRWYGRGWIRRPFRRRFRRKRRIQYPIPRNEHETTSL |
| | QSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID |
| | NO: 59) |
| ORF1/2 | MPYYYRRRRYNYRRPRWYGRGWIRRPFRRRFRRKRRSQIDSNCSNKPSNAC |
| | KSRKKRRRKKKKKYSSSSATSDSSSSCTESE (SEQ ID NO: 60) |

TABLE N15
Exemplary Anellovirus nucleic acid sequence (Gammatorquevirus)

| | |
|---|---|
| Name | TTMDV-MD1-073 |
| Genus/Clade | Gammatorquevirus |
| Accession Number | AB290918.1 |
| Full Sequence: 3242 bp | |

(SEQ ID NO: 61)
Annotations:

| Putative Domain | Base range |
|---|---|
| TATA Box | 21-25 |
| Cap Site | 42-49 |
| Transcriptional Start Site | 49 |
| 5′ UTR Conserved Domain | 117-187 |
| ORF2 | 283-588 |
| ORF2/2 | 283-584; 1977-2388 |
| ORF2/3 | 283-584; 2197-2614 |
| ORF1 | 432-2453 |
| ORF1/1 | 432-584; 1977-2453 |
| ORF1/2 | 432-584; 2197-2388 |
| Three open-reading frame region | 2186-2385 |
| Poly(A) Signal | 2676-2681 |
| GC-rich region | 3054-3172 |

TABLE A15
Exemplary Anellovirus amino acid sequences (Gammatorquevirus)
TTMDV-MD1-073 (Gammatorquevirus)

| | |
|---|---|
| ORF2 | MWMSGIADSHDSWCDCDTPFAHLLASIFPPGHTDRTRTIQEILTRDFRKTCL |
| | SGGADATNSGMAETIEEKREDFQKEEKEDFTEEQNIEDLLAAVADAEGR |
| | (SEQ ID NO: 62) |
| ORF2/2 | MWMSGIADSHDSWCDCDTPFAHLLASIFPPGHTDRTRTIQEILTRDFRKTCL |
| | SGGADATNSGMAETIEEKREDFQKEEKEDFTEEQNIEDLLAAVADAEGRYQ |
| | TNQLKTQETKTNMMCPIQSKKHYKLLTQQKTLLPRCSMTGTTDGVALHQQ |
| | LLKECNKTSQLIHLSNLIQTQNQHPRKKDYYQSSTTHKRKRKRSTNVSSLSA |
| | KKVHARSRKRRKTSSSSSSSSSSSSRNSSTTS (SEQ ID NO: 63) |
| ORF2/3 | MWMSGIADSHDSWCDCDTPFAHLLASIFPPGHTDRTRTIQEILTRDFRKTCL |
| | SGGADATNSGMAETIEEKREDFQKEEKEDFTEEQNIEDLLAAVADAEGRTST |
| | QEKKTTTSPPRPTKENGKDQPMSPLSLRRKYMPGAGNGGKHPQAHPAAAA |
| | AAAETQAQPLSTNQGLKSETKIITTTNGGTRITLTRFKPGFEQETEKELAQAF |
| | NRPPRLFKEDKPFYPWLPRFTPLVNFHLNFKG (SEQ ID NO: 64) |
| ORF1 | MPFWWGRRNKFWYGRNYRRKKRRFPKRRKRRFYRRTKYRRPARRRRRRR |
| | RKVRRKKKTLIVRQWQPDSIVLCKIKGYDSIIWGAEGTQFQCSTHEMYEYTR |
| | QKYPGGGGFGVQLYSLEYLYDQWKLRNNIWTKTNQLKDLCRYLKCVMTF |
| | YRHQHIDFVIVYERQPPFEIDKLTYMKYHPYMLLQRKHKIILPSQTTNPRGKL |
| | KKKKTIKPPKQMLSKWFFQQQFAKYDLLLIAAAACSLRYPRIGCCNENRMIT |
| | LYCLNTKFYQDTEWGTTKQAPHYFKPYATINKSMIFVSNYGGKKTEYNIGQ |
| | WIETDIPGEGNLARYYRSISKEGGYFSPKILQAYQTKVKSVDYKPLPIVLGRY |
| | NPAIDDGKGNKIYLQTIMNGHWGLPQKTPDYIIEEVPLWLGFWGYYNYLKQ |
| | TRTEAIFPLHMFVVQSKYIQTQQTETPNNFWAFIDNSFIQGKNPWDSVITYSE |
| | QKLWFPTVAWQLKTINAICESGPYVPKLDNQTYSTWELATHYSFHFKWGGP |
| | QISDQPVEDPGNKNKYDVPDTIKEALQIVNPAKNIAATMFHDWDYRRGCITS |
| | TAIKRMQQNLPTDSSLESDSDSEPAPKKKRLLPVLHDPQKKTEKINQCLLSL |
| | CEESTCQEQETEENILKLIQQQQQQQQKLKHNLLVLIKDLKVKQRLLQLQTG |
| | VLE (SEQ ID NO: 65) |
| ORF1/1 | MPFWWGRRNKFWYGRNYRRKKRRFPKRRKRRFYRRTKYRRPARRRRRRR |
| | RKISDQPVEDPGNKNKYDVPDTIKEALQIVNPAKNIAATMFHDWDYRRGCI |
| | TSTAIKRMQQNLPTDSSLESDSDSEPAPKKKRLLPVLHDPQKKTEKINQCLLS |
| | LCEESTCQEQETEENILKLIQQQQQQQQKLKHNLLVLIKDLKVKQRLLQLQT |
| | GVLE (SEQ ID NO: 66) |
| ORF1/2 | MPFWWGRRNKFWYGRNYRRKKRRFPKRRKRRFYRRTKYRRPARRRRRRR |
| | RKISDQPVEDPGNKNKYDVPDTIKEALQIVNPAKNIAATMFHDWDYRRGCI |
| | TSTAIKRMQQNLPTDSSLESDSDSEPAPKKKRLLPVLHDPQKKTEKINQCLLS |
| | LCEESTCQEQETEENILKLIQQQQQQQQKLKHNLLVLIKDLKVKQRLLQLQT |
| | GVLE (SEQ ID NO: 67) |

TABLE N16
Exemplary Anellovirus nucleic acid sequence (Gammatorquevirus)

| | |
|---|---|
| Name | Ring3.1 |
| Genus/Clade | Gammatorquevirus |
| Accession Number | |
| Full Sequence: 3264 bp | |

(SEQ ID NO: 878)
Annotations:

| Putative Domain | Base range |
|---|---|
| TATA Box | 87-93 |
| Cap Site | 110-117 |
| Transcriptional Start Site | 117 |
| 5′ UTR Conserved Domain | 185-255 |
| ORF2 | 285-671 |
| ORF2/2 | 285-667; 2063-2498 |
| ORF2/3 | 285-667; 2295-2697 |
| TAIP | 385-585 |
| ORF1 | 512-2545 |
| ORF1/1 | 512-667; 2063-2545 |
| ORF1/2 | 512-667; 2295-2498 |
| Three open-reading frame region | 2295-2495 |
| Poly(A) Signal | 2729-2734 |
| GC-rich region | 3141-3264 |

TABLE A16
Exemplary Anellovirus amino acid sequences (Gammatorquevirus)
Ring 3.1 (Gammatorquevirus)

| | |
|---|---|
| ORF2 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERR (SEQ ID NO: 879) |
| ORF2/2 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERSGVVHKSQTQLLKTHAAETNILSPIQCNKQYKLKTLKSCTQQPS |
| | SMTGTLEGASLHKQLLKECQKTSKLIHLSNLMAQNHPKKRKDAPKKSQHK |
| | TKSKKRSKNVSSHSAKSLHAKKKQRTSSSSSSSSSSSSTSSEKTSSNSSLT |
| | (SEQ ID NO: 880) |
| ORF2/3 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERRITQKKEKMHQRNPNTKPKARRDPRMSPLTLRRAYMPRRNRG |
| | PPALHPAAAAAAVQAQKKPLQTPHSPEKRTENKSTTNGTFRVIPFKPGFEQE |
| | TEKELAIAFCRPPRKYKNDPPFYPWLPWTPLVHFNLNYKG (SEQ ID NO: |
| | 881) |
| TAIP | MDMTTFVTATVLLLTCLLIFFLLVIKTGILPLIKYLLEILQKHAILVETKEQAV |
| | VGSPLPLPPLQQI (SEQ ID NO: 882) |
| ORF1 | MPFWWRRRNKRWWGRRFRYRRYNKYKTRRRRRIPRRRNRRFTKTRRRRK |
| | RKKVRRKLKKITIKQWQPDSVKKCKIKGYSTLVMGAQGKQYNCYTNQASD |
| | YVQPKAPQGGGFGCEVFNLKWLYQEYTAHRNIWTKTNEYTDLCRYTGAQII |
| | LYRHPDVDFIVSWDNQPPFLLNKYTYPELQPQNLLLARRKRIILSQKSNPKG |
| | KLRIKLRIPPPKQMITKWFFQRDFCDVNLFKLCASAASFRYPGISHGAQSTIFS |
| | AYALNTDFYQCSDWCQTNTETGYLNIKTQQMPLWFHYREGGKEKWYKYT |
| | NKEHRPYTNTYLKSISYNDGLFSPKAMFAFEVKAGGEGTTEPPQGAQLIANL |
| | PLIALRYNPHEDTGHGNEIYLTSTFKGTYDKPKVTDALYFNNVPLWMGFYG |
| | YWDFILQETKNKGVFDQHMFVVKCPALRPISQVTKQVYYPLVDMDFCSGR |
| | LPFDEYLSKDIKSHWYPTAERQTVTINNFVTAGPYMPKFEPTDKDSTWQLN |
| | YHYKFFFKWGGPQVTDPTVEDPCSRNKYPVPDTMQQTIQIKNPEKLHPATL |
| | FHDWDLRRGFITQAAIKRMSENLQIDSSFESDGTESPKKKKRCTKEIPTQNQ |
| | KQEEIQECLLSLCEEPTCQEETEDLQLFIQQQQQQQYKLRKNLFKLLTHLKK |
| | GQRISQLQTGLLE (SEQ ID NO: 883) |
| ORF1/1 | MPFWWRRRNKRWWGRRFRYRRYNKYKTRRRRRIPRRRNRRFTKTRRRRK |
| | RKKWGGPQVTDPTVEDPCSRNKYPVPDTMQQTIQIKNPEKLHPATLFHDWD |
| | LRRGFITQAAIKRMSENLQIDSSFESDGTESPKKKKRCTKEIPTQNQKQEEIQE |
| | CLLSLCEEPTCQEETEDLQLFIQQQQQQQYKLRKNLFKLLTHLKKGQRISQL |
| | QTGLLE (SEQ ID NO: 884) |
| ORF1/2 | MPFWWRRRNKRWWGRRFRYRRYNKYKTRRRRRIPRRRNRRFTKTRRRRK |
| | RKKNHPKKRKDAPKKSQHKTKSKKRSKNVSSHSAKSLHAKKKQRTSSSSSS |
| | SSSSSSTSSEKTSSNSSLT (SEQ ID NO: 885) |

TABLE N17
Exemplary Anellovirus nucleic acid sequence (Gammatorquevirus)

| | |
|---|---|
| Name | Ring4 |
| Genus/Clade | Gammatorquevirus |
| Accession Number | |
| Full Sequence: 3176 bp | |

(SEQ ID NO: 886)
Annotations:

| Putative Domain | Base range |
|---|---|
| TATA Box | 87-93 |
| Cap Site | 110-117 |
| Transcriptional Start Site | 117 |
| 5′ UTR Conserved Domain | 185-254 |
| ORF2 | 286-660 |
| ORF2/2 | 286-656; 1998-2442 |
| ORF2/3 | 286-656; 2209-2641 |
| TAIP | 385-484 |
| ORF1 | 501-2489 |
| ORF1/1 | 501-656; 1998-2489 |
| ORF1/2 | 501-656; 2209-2442 |
| Three open-reading frame region | 2209-2439 |
| Poly(A) Signal | 2672-2678 |
| GC-rich region | 3076-3176 |

TABLE A17
Exemplary Anellovirus amino acid sequences (Gammatorquevirus)
Ring 4 (Gammatorquevirus)

| | |
|---|---|
| ORF2 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFER (SEQ ID NO: 887) |
| ORF2/2 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFESGVDHNSMNQKLLTLANKSSMMSPILSTKQYKL |
| | KIQKDKTPDLSSMIGTTDEALLKKDLLKECQLTSQLIQISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 888) |
| ORF2/3 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFERSASNFRGRHSQKEKENWTPTHSPTTKRRGDTV |
| | MSPLSLQKRYLPRNRDTRRPPAAHQAAAGAAAPPQEKHPPAHPQTKRESTN |
| | ASASHRHVTLTRFKPGFEEQTERELAIIFHRPPRTYKEDLPFYPWLPPAPLVQ |
| | FNLNFKG (SEQ ID NO: 889) |
| TAIP | MRRRRTKYGCLKLLTLMIISAVAGIHLLTFLLPYFLLATKIVILLLTKFF (SEQ |
| | ID NO: 890) |
| ORF1 | MPFWWRRRRKFWTNNRFNYTKRRRYRKRWPRRRRRRRPYRRPVRRRRRK |
| | LRKVKRKKKSLIVRQWQPDSIRTCKIIGQSAIVVGAEGKQMYCYTVNKLINV |
| | PPKTPYGGGFGVDQYTLKYLYEEYRFAQNIWTQSNVLKDLCRYINVKLIFY |
| | RDNKTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNENPQ |
| | VFFYYLNHGYYTITNWGAQSSTAYRPNSKVTDTTYYRYKNDRKNINIKSHE |
| | YEKSISYENGYFQSSFLQTQCIYTSERGEACIAEKPLGIAIYNPVKDNGDGNM |
| | IYLVSTLANTWDQPPKDSAILIQGVPIWLGLFGYLDYCRQIKADKTWLDSHV |
| | LVIQSPAIFTYPNPGAGKWYCPLSQSFINGNGPFNQPPTLLQKAKWFPQIQYQ |
| | QEIINSFVESGPFVPKYANQTESNWELKYKYVFTFKWGGPQFHEPEIADPSK |
| | QEQYDVPDTFYQTIQIEDPEGQDPRSLIHDWDYRRGFIKERSLKRMSTYFST |
| | HTDQQATSEEDIPKKKKRIGPQLTVPQQKEEETLSCLLSLCKKDTFQETETQE |
| | DLQQLIKQQQEQQLLLKRNILQLIHKLKENQQMLQLHTGMLP (SEQ ID NO: |
| | 891) |
| ORF1/1 | MPFWWRRRRKFWTNNRFNYTKRRRYRKRWPRRRRRRRPYRRPVRRRRRK |
| | LRKWGGPQFHEPEIADPSKQEQYDVPDTFYQTIQIEDPEGQDPRSLIHDWDY |
| | RRGFIKERSLKRMSTYFSTHTDQQATSEEDIPKKKKRIGPQLTVPQQKEEETL |
| | SCLLSLCKKDTFQETETQEDLQQLIKQQQEQQLLLKRNILQLIHKLKENQQM |
| | LQLHTGMLP (SEQ ID NO: 892) |
| ORF1/2 | MPFWWRRRRKFWTNNRFNYTKRRRYRKRWPRRRRRRRPYRRPVRRRRRK |
| | LRKISKQLQRKTFPKRKRELDPNSQSHNKKKRRHCHVSSLSAKKIPSKKQRH |
| | KKTSSSSSSSSRSSSSSSRETSSSSSTN (SEQ ID NO: 893) |

TABLE N18
Exemplary Anellovirus nucleic acid sequence
(Alphatorquevirus) - Clade 1

| | |
|---|---|
| Name | Ring5.2 |
| Genus/Clade | Alphaatorquevirus Clade 1 |
| Accession Number | |
| Full Sequence: 3696 bp | |

(SEQ ID NO: 894)
Annotations:

| Putative Domain | Base range |
|---|---|
| TATA Box | 85-91 |
| Cap Site | 108-115 |
| Transcriptional Start Site | 115 |
| 5′ UTR Conserved Domain | 178-248 |
| ORF2 | 300-692 |
| ORF2/2 | 300-688; 2282-2804 |
| ORF2/3 | 300-688; 2484-2976 |
| ORF2t/3 | 300-349: 2484-2976 |
| TAIP | 322-471 |
| ORF1 | 572-2758 |
| ORF1/1 | 572-688; 2282-2758 |
| ORF1/2 | 572-688; 2484-2804 |
| Three open-reading frame region | 2484-2755 |
| Poly(A) Signal | 3018-3023 |
| GC-rich region | 3555-3696 |

TABLE A18
Exemplary Anellovirus amino acid sequences (Alphatorquevirus) Clade 1
Ring 5.2 (Alphaatorquevirus) Clade 1

| | |
|---|---|
| ORF2 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDL (SEQ ID NO: 895) |
| ORF2/2 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDFQSTTPASREPTRFPTPINTLASYKS |
| | RTRNYSDRGQCSTSGTSDVGSLAKEVLRECQNTQAMMNLLRQVSHQSETSS |
| | TRRSEEKIESKKNAILSSKRSRKKRPQKKKNQHPKKKPRKRSYSTSSSSRDAT |
| | SESSDEGSSSSLQTSSDSARESTGTRSSHSAPTLHTRPAFSQYW (SEQ ID NO: |
| | 896) |
| ORF2/3 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDLSPIKAKQARLGVPRRKSRAKRMLF |
| | SPQSARGRRDPRRRRTSTPRKSPERGATPPAPAPETPPASPQTRAQARLYRHP |
| | PTPPGSPLEPGAHIAPPPYIPDLLFPNTGKKKKFSPFDWETEAQIAGWMRRPM |
| | RFYPSDTPHYPWLPPERDIPKICNINFKIKLQ (SEQ ID NO: 897) |
| ORF2t/3 | MPWRPPVHSVQGREDQWSPIKAKQARLGVPRRKSRAKRMLFSPQSARGRR |
| | DPRRRRTSTPRKSPERGATPPAPAPETPPASPQTRAQARLYRHPPTPPGSPLEP |
| | GAHIAPPPYIPDLLFPNTGKKKKFSPFDWETEAQIAGWMRRPMRFYPSDTPH |
| | YPWLPPERDIPKICNINFKIKLQE (SEQ ID NO: 898) |
| TAIP | IVSRGERISGSQAFFTATIRFAAAVTLLAILIALLIAFLAPVHQGPLRG (SEQ ID |
| | NO: 899) |
| ORF1 | TAWWWGRWRRRWRRRRPYTTRLRRRRARRAFPRRRRRRFVSRRWRRPYR |
| | RRRRRGRRRRRRRRRHKPTLILRQWQPDCIRHCKITGWMPLIICGKGSTQFN |
| | YITHADDITPRGASYGGNFTNMTFSLEAIYEQFLYHRNRWSASNHDLELCRY |
| | KGTTLKLYRHPEVDYIVTYSRTGPFEISHMTYLSTHPMLMLLNKHHIVVPSL |
| | KTKPRGRKAIKVRIRPPKLMNNKWYFTRDFCNIGLFQLWATGLELRNPWLR |
| | MSTLSPCIGFNVLKNSIYTNLSNLPQYKNERLNIINNILHPQEITGTNNKKWQ |
| | YTYTKLMAPIYYSANRASTYDWENYSKETNYNNTYVKFTQKRQEKLTKIR |
| | KEWQMLYPQQPTALPDSYDLLQEYGLYSPYYLNPTRINLDWMTPYTHVRY |
| | NPLVDKGFGNRIYIQWCSEADVSYNRTKSKCLLQDMPLFFMCYGYIDWAIK |
| | NTGVSSLVKDARICIRCPYTEPQLVGSTEDIGFVPISETFMRGDMPVLAPYIPL |
| | SWFCKWYPNIAHQKEVLESIISCSPFMPRDQDMNGWDITIGYKMDFLWGGS |
| | PLPSQPIDDPCQQGTHPIPDPDKHPRLLQVSNPKLLGPRTVFHKWDIRRGQFS |
| | KRSIKRVSEYSSDDESLAPGLPSKRNKLDSAFRGENREQKECYSLLKALEEE |
| | ETPEEEEPAPQEKAQKEELLHQLQLQRRHQRVLRRGLKLVFTDILRLRQGVH |
| | WNPELT (SEQ ID NO: 900) |
| ORF1/1 | TAWWWGRWRRRWRRRRPYTTRLRRRRARRAFPRRRRRRFPIDDPCQQGT |
| | HPIPDPDKHPRLLQVSNPKLLGPRTVFHKWDIRRGQFSKRSIKRVSEYSSDDE |
| | SLAPGLPSKRNKLDSAFRGENREQKECYSLLKALEEEETPEEEEPAPQEKAQ |
| | KEELLHQLQLQRRHQRVLRRGLKLVFTDILRLRQGVHWNPELT (SEQ ID |
| | NO: 901) |
| ORF1/2 | TAWWWGRWRRRWRRRRPYTTRLRRRRARRAFPRRRRRRFVSHQSETSSTR |
| | RSEEKIESKKNAILSSK |
| | RSRKKRPQKKKNQHPKKKPRKRSYSTSSSSRDATSESSDEGSSSSLQTSSDSA |
| | RESTGTRSSHSAPTLHTRPAFSQYW (SEQ ID NO: 902) |

TABLE N19
Exemplary Anellovirus nucleic acid sequence
(Alphatorquevirus) - Clade 3

| | |
|---|---|
| Name | Ring 6.0 |
| Genus/Clade | Alphatorquevirus - Clade 3 |
| Accession Number | |
| Full Sequence: 3828 bp | |

(SEQ ID NO: 903)
Annotations:

| Putative Domain | Base range |
|---|---|
| TATA Box | 85-92 |
| Cap Site | 109-116 |
| Transcriptional Start Site | 116 |
| 5′ UTR Conserved Domain | 176-246 |
| ORF2 | 351-710 |
| ORF2/2 | 351-706; 2360-2825 |
| ORF2/3 | 351-706; 2556-3060 |
| TAIP | 373-528 |
| ORF1 | 581-2884 |
| ORF1/1 | 581-706; 2360-2884 |
| ORF1/2 | 581-706; 2556-2825 |
| Three open-reading frame region | 2556-2821 |
| Poly(A) Signal | 3055-3061 |
| GC-rich region | 3720-3828 |

TABLE A19
Exemplary Anellovirus amino acid sequences (Alphatorquevirus)-Clade 3
Ring 6.0 (Alphatorquevirus)

| | |
|---|---|
| ORF2 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQ (SEQ ID NO: 904) |
| ORF2/2 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQLLETPARPPKVPRAIECLDQYKSLTRGTTRONS |
| | PYTRGTSDMGSLAEKLLRECKNNQYLMTLFQQGSSAVAEIQKHSNAAKKS |
| | NKKKTYFSQSSSSSESPRGRPRKRAKARKKTRKNRRPSPSNSETSCTSSGSW |
| | ESNSDRSSTKCRGSNKINT (SEQ ID NO: 905) |
| ORF2/3 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQVQAQSPRYRSTPMQPRRATKRKLTFPSPAAQA |
| | SPPVGDLAREPKRGRKLAKTGDPLPATQRPAAQAAAHGRATPIAPLPNAEG |
| | PTKSTHKPYVIAKGSGINFYFSQCNIDMFGDPKPYKPSSNDWKEEYEAAKY |
| | WDRPPRRDLRSTPFYPWAPTPKPYNVNFALNYK (SEQ ID NO: 906) |
| TAIP | MRSIFNVTGSTVAFTPTLLCAAVLILLVISIISLLCSAVRKTRTLLRHPGL (SEQ |
| | ID NO: 907) |
| ORF1 | MAYGWWRRRRRRPWWRRRWRRWRRRRRPRRRRPRRRYRRRRTVRRRGR |
| | GRWTRAHRRWRRKGKRSRKKKIIIRQWQPNYTRRCNIVGYMPLLICGENTV |
| | ATNYATHSDDSYYPGPFGGGMTTDKFTLRILYDEYKRFMNYWTSSNEDLD |
| | LCRYLGCTLYVFRHPEVDFIIIINTSPPFLDTEITGPSIHPGMMALNKRSRWIPS |
| | IKNRPGRKHYIKIKVGAPRMFTDKWYPQTDLCDMTLLTIFASAADMQYPFG |
| | SPLTDTIVVSFQVLQSMYNDCLSVLPDNFAETSGKGTQLHENIIQHLPYYNTT |
| | QTQAQFKRFIENMNATNGDNIWASYINTTKFSSANTPKNDTGIGGPYTTYSD |
| | SWYKGTVYNDKIKTIPIKASKLYYEQTKNLIGITFTGSTHRLHYCGGLYSSV |
| | WLSAGRSYFETKGPYTDITYNPFSDRGEGNMLWIDWLTKNDSVYSKTSSKC |
| | LIENLPLWASVYGYKEYCSKVTGDTNIEHNCRCVIRSPYTVPQLLDHNNPFR |
| | GYVPYSFNFGNGKMPGGSSLVPIRMRAKWYPTLFHQKEVLEAIAQAGPFAY |
| | HSDIKKVSLGIKYRFKWVWGGNPVSQQVVRNPCKTTQGSSGNRVPRSIQVV |
| | DPRYNTPELTIHAWDFRHGFFGRKAIKRMQEQPIPHDTFSAGFKRSRRDTEA |
| | LQCSQEEQQKENLLFPVQQLKRVPPWETSQESQSEEENSQKQETLSQQLRD |
| | QLHKQRLMGEQLRSLLYQMQRVQQNQHINPMLLPKGLALTSISHNVI (SEQ |
| | ID NO: 908) |
| ORF1/1 | MAYGWWRRRRRRPWWRRRWRRWRRRRRPRRRRPRRRYRRRRTVVRNPC |
| | KTTQGSSGNRVPRSIQVVDPRYNTPELTIHAWDFRHGFFGRKAIKRMQEQPI |
| | PHDTFSAGFKRSRRDTEALQCSQEEQQKENLLFPVQQLKRVPPWETSQESQS |
| | EEENSQKQETLSQQLRDQLHKQRLMGEQLRSLLYQMQRVQQNQHINPMLL |
| | PKGLALTSISHNVI (SEQ ID NO: 909) |
| ORF1/2 | MAYGWWRRRRRRPWWRRRWRRWRRRRRPRRRRPRRRYRRRRTGSSAVA |
| | EIQKHSNAAKKSNKKKTYFSQSSSSSESPRGRPRKRAKARKKTRKNRRPSPS |
| | NSETSCTSSGSWESNSDRSSTKCRGSNKINT (SEQ ID NO: 910) |

TABLE N20
Exemplary Anellovirus nucleic acid sequence
(Alphatorquevirus) - Clade 7

| | |
|---|---|
| Name | Ring7 |
| Genus/Clade | Alphatorquevirus - Clade 7 |
| Accession Number | |
| Full Sequence: 3815 bp | |

(SEQ ID NO: 911)
Annotations:

| Putative Domain | Base range |
|---|---|
| TATA Box | 82-87 |
| Cap Site | 103-110 |
| Transcriptional Start Site | 110 |
| 5′ UTR Conserved Domain | 170-240 |
| ORF2 | 351-740 |
| ORF2/2 | 351-737; 2378-2843 |
| ORF2/3 | 351-737; 2526-3057 |
| TAIP | 379-543 |
| ORF1 | 614-2911 |
| ORF1/1 | 614-737; 2378-2911 |
| ORF1/2 | 614-737; 2526-2843 |
| Three open-reading frame region | 2526-2840 |
| Poly(A) Signal | 3056-3062 |
| GC-rich region | 3716-3815 |

TABLE A20
Exemplary Anellovirus amino acid sequences (Alphatorquevirus)-Clade 7
Ring7.0 (Alphatorquevirus)

| | |
|---|---|
| ORF2 | MSIWRPPLGNVSYRERNWLQAVETSHSSFCGCGDFILHLTNLAARFALQGPP |
| | PEGGPPRPRPPLLRALPAPEVRRETRTENRGASGEPWPGDGGGRDDGAAAG |
| | GPADGGDAYDAGDLDDLFAAVEEEQQ (SEQ ID NO: 912) |
| ORF2/2 | MSIWRPPLGNVSYRERNWLQAVETSHSSFCGCGDFILHLTNLAARFALQGPP |
| | PEGGPPRPRPPLLRALPAPEVRRETRTENRGASGEPWPGDGGGRDDGAAAG |
| | GPADGGDAYDAGDLDDLFAAVEEEQQLSRTRVTESSPSLPIPIDNVATYKLL |
| | THQPWARSTPSTHGTGDGGFLVQMLSRECHKNREMLSALQTLSRDPDIFPR |
| | QTEKTTDKKKTSLYRKKDGAHPQKKPRTRRAPRKARRSYSSSSSSGSSQSTS |
| | RSSSDSESNSDTSSKKSSKRKRVST (SEQ ID NO: 913) |
| ORF2/3 | MSIWRPPLGNVSYRERNWLQAVETSHSSFCGCGDFILHLTNLAARFALQGPP |
| | PEGGPPRPRPPLLRALPAPEVRRETRTENRGASGEPWPGDGGGRDDGAAAG |
| | GPADGGDAYDAGDLDDLFAAVEEEQQCYPESVTKTGRCSPLYKPFQETQIS |
| | SPDRQRRLPTRRRLRFTGKKTAHIHRRSPGRGEPPGKRAAPTAAAAAAAALS |
| | PPRGAAATRSPTPIHPPRSPQNASGSPPKPPIIRPATNKVYLFEPSKGLLPIVGK |
| | EAWEDEYCTCKYWDRPPRTNHLDIPTYPWMPTNFKVSFKLGFKP (SEQ ID |
| | NO: 914) |
| TAIP | MSPTGREIGFRPSKHPTVLFVAVVILFFILLIWLHALLSRGPRQRVVHLGRGR |
| | RS (SEQ ID NO: 915) |
| ORF1 | MAWRWWWQRRWRRRRWPRRRWRRLRRRRPRRPVRRRRRRTTVRRRRW |
| | RGRRGRRTYTRRAVRRRRRPRKRLVLTQWSPQTVRNCSIRGIVPMVICGHT |
| | KAGRNYAIHSEDFTTQIQPFGGSFSTTTWSLKVLWDEHQKFQNRWSYPNTQ |
| | LDLARYRGVTFWFYRDQKTDYIVQWSRNPPFKLNKYSSAMYHPGMMMQA |
| | KRKLVVPSFQTRPKGKKRYRVTIKPPNMFADKWYTQEDLCPVPLVQIVVSA |
| | ASLLHPFCPPQTNNPCITFQVLKDIYDECIGVNETMKDKYKKLQTTLYTTCT |
| | YYQTTQVLAQLSPAFQPAMKPTTTQSAATATTLGNYVPELKYNNGSFHTGQ |
| | NAVFGMCSYKPTDSIMTKANGWFWQNLMVDNNLHSSYGKATLECMEYHT |
| | GIYSSIFLSPQRSLEFPAAYQDVTYNPNCDRAVGNVVWFQYSTKMDTNFDE |
| | TKCKCVLKNIPLWAAFNGYSDFIMQELSISTEIHNFGIVCFQCPYTFPPCENK |
| | NKPLKGYVFYDTTFGNGKMPDGSGHVPIYWQQRWWIRLAFQVQVMHDFV |
| | LTGPFSYKDDLANTTLTARYKFKFKWGGNIIPEQIIKNPCHREQSLASYPDRQ |
| | RRDLQVVDPSTMGPIYTFHTWDWRRGLFGADAIQRVSQKPGDALRFTNPFK |
| | RPRYLPPTDREDYRQEEDFALQEKRRRTSTEEAQDEESPPESAPLLQQQQQQ |
| | RQLSVHLAEQQRLGVQLRYILQEVLKTQAGLHLNPLLLGPPQTRSISLSPPKA |
| | YSP (SEQ ID NO: 916) |
| ORF1/1 | MAWRWWWQRRWRRRRWPRRRWRRLRRRRPRRPVRRRRRRTTIIKNPCHR |
| | EQSLASYPDRQRRDLQVVDPSTMGPIYTFHTWDWRRGLFGADAIQRVSQKP |
| | GDALRFTNPFKRPRYLPPTDREDYRQEEDFALQEKRRRTSTEEAQDEESPPE |
| | SAPLLQQQQQQRQLSVHLAEQQRLGVQLRYILQEVLKTQAGLHLNPLLLGP |
| | PQTRSISLSPPKAYSP (SEQ ID NO: 917) |
| ORF1/2 | MAWRWWWQRRWRRRRWPRRRWRRLRRRRPRRPVRRRRRRTTMLSRECH |
| | KNREMLSALQTLSRDPDIFPRQTEKTTDKKKTSLYRKKDGAHPQKKPRTRR |
| | APRKARRSYSSSSSSGSSQSTSRSSSDSESNSDTSSKKSSKRKRVST (SEQ ID |
| | NO: 918) |

Ring9 is an Anellovirus that was isolated from whole blood. In some embodiments, a method described herein comprises delivering an Anellovector (e.g., an Anellovector having sequence similarity to Ring9) to blood cells (e.g., to the blood cells of a subject).

TABLE N21
Exemplary Anellovirus nucleic acid sequence (Betatorquevirus)

| | |
|---|---|
| Name | Ring9 |
| Genus/Clade | Betatorquevirus |
| Accession Number | MH649263.1 |
| Full Sequence: 2845 bp | |

(SEQ ID NO: 1001)
Annotations:

| Putative Domain | Base range |
|---|---|
| TATA Box | 142-148 |
| Initiation Element | 162-177 |
| Transcriptional Start Site | 172 |
| 5′ UTR Conserved Domain | 226-296 |
| ORF2 | 328-651 |
| ORF2/2 | 328-647; 2121-2457 |
| ORF2/3 | 328-647; 2296-2680 |
| ORF1 | 510-2477 |
| ORF1/1 | 510-647; 2121-2477 |
| ORF1/2 | 510-647; 2296-2457 |
| Three open-reading frame region | 2296-2454 |
| GC-rich region | 2734-2845 |

TABLE A21
Exemplary Anellovirus amino acid sequences (Betatorquevirus)
Ring9 (Betatorquevirus)

| | |
|---|---|
| ORF2 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEEN |
| | CG (SEQ ID NO: 1002) |
| ORF2/2 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEEN |
| | CGPIGLFPITSMKQYKSRIQTHAHKQNSKNGTGDVILLQKKLSKELDNTRNL |
| | MKLCKSLQVPNTTHQYTDKHHRGRTQKRTRKRKKTKHKRSRSSSTSSESIN |
| | SISSSSSSST (SEQ ID NO: 1003) |
| ORF2/3 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEEN |
| | CGFQTQPTSTQTNITVDGLRNGLGRGKRPNTRDPDPAQQAQKASTASQAAA |
| | QAVPETPKYRIVASNIKVELFPTKKPFKNRRFTPSERETERQCAKAFCRPERH |
| | FFYDPPFYPYCVPEPIVNFALGYKI (SEQ ID NO: 1004) |
| ORF1 | MPPYWRQKYYRRRYRPFSWRTRRIIQRRKRWRYRKPRKTYWRRKLRVRKR |
| | FYKRKLKKIVLKQFQPKIIRRCTIFGTICLFQGSPERANNNYIQTIYSYVPDKE |
| | PGGGGWTLITESLSSLWEDWEHLKNVWTQSNAGLPLVRYGGVTLYFYQSA |
| | YTDYIAQVENCYPMTDTKYTHADSAPNRMLLKKHVIRVPSRETRKKRKPYK |
| | RVRVGPPSQMONKWYFORDICEIPLIMIAATAVDFRYPFCASDCASNNLTLT |
| | CLNPLLFQNQDFDHPSDTQGYFPKPGVYLYSTQRSNKPSSSDCIYLGNTKDN |
| | QEGKSASSLMTLKTQKITDWGNPFWHYYIDGSKKIFSYFKPPSQLDSSDFEH |
| | MTELAEPMFIQVRYNPERDTGQGNLIYVTENFRGQHWDPPSSDNLKLDGFP |
| | LYDMCWGFIDWIEKVHETENLLTNYCFCIRSSAFNEKKTVFIPVDHSFLTGFS |
| | PYETPVKSSDQAHWHPQIRFQTKSINDICLTGPGCARSPYGNYMQAKMSYK |
| | FHVKWGGCPKTYEKPYDPCSQPNWTIPHNLNETIQIQNPNTCPQTELQEWD |
| | WRRDIVTKKAIERIRQHTEPHETLQISTGSKHNPPVHRQTSPWTDSETDSEEE |
| | KDQTQEIQIQLNKLRKHQQHLKQQLKQYLKPQNIE (SEQ ID NO: 1005) |
| ORF1/1 | MPPYWRQKYYRRRYRPFSWRTRRIIQRRKRWRYRKPRKTYWRRKLRPNW |
| | TIPHNLNETIQIQNPNTCPQTELQEWDWRRDIVTKKAIERIRQHTEPHETLQIS |
| | TGSKHNPPVHRQTSPWTDSETDSEEEKDQTQEIQIQLNKLRKHQQHLKQQL |
| | KQYLKPQNIE (SEQ ID NO: 1006) |
| ORF1/2 | MPPYWRQKYYRRRYRPFSWRTRRIIQRRKRWRYRKPRKTYWRRKLRVPNT |
| | THQYTDKHHRGRTQKRTRKRKKTKHKRSRSSSTSSESINSISSSSSSST (SEQ |
| | ID NO: 1007) |

Ring10 is an Anellovirus that was isolated from lung tissue. In some embodiments, a method described herein comprises delivering an Anellovector (e.g., an Anellovector having sequence similarity to Ring10) to lung tissue (e.g., to the lung of a subject).

TABLE N22
Exemplary Anellovirus nucleic acid sequence (Betatorquevirus)

| | |
|---|---|
| Name | Ring10 |
| Genus/Clade | Betatorquevirus |
| Accession Number | JX134044.1 |
| Full Sequence: 2912 bp | |

(SEQ ID NO: 1008)
Annotations:

| Putative Domain | Base range |
|---|---|
| TATA Box | 152-158 |
| Initiation Element | 172-187 |
| Transcriptional Start Site | 182 |
| 5′ UTR Conserved Domain | 239-309 |
| ORF2 | 343-633 |
| ORF2/2 | 343-629; 2196-2505 |
| ORF2/3 | 343-629; 2371-2734 |
| ORF1 | 522-2540 |
| ORF1/1 | 522-629; 2196-2540 |
| ORF1/2 | 522-629; 2371-2505 |
| Three open-reading frame region | 2276-2502 |
| GC-rich region | 2803-2912 |

TABLE A22
Exemplary Anellovirus amino acid sequences (Betatorquevirus)
Ring10 (Betatorquevirus)

| | |
|---|---|
| ORF2 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAG (SEQ ID |
| | NO: 1009) |
| ORF2/2 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAGQHMLFPI |
| | TSMKQLRYRIQPPDQSTSCTPLTKGGDNLQKKLQNACLKTGKLKKLLYCLQ |
| | NTDSRSQHKHKPHKRTRPRKKKKRATSSSDSSDSEPSSSSSSAE (SEQ ID NO: |
| | 1010) |
| ORF2/3 | MFKSPTYLTTKGKNNALINCFVGDHDLLCSCNNPAYHCLQILATTLAPQLK |
| | QEEKQQIIQCLGGTDAVATTRGDEEIGLEDLEKLFTEDTEEDAAGIQIRGANT |
| | NTSPTRGPVLGRRRREQPLRATPPTANQAAPAQAQNNTNIERPTKIRITNSKN |
| | TVYLFPPEQKNRRLTPWEIQEDKEIANLFGRPHRYFLKDIPFYWDIPPEPKVN |
| | FDLNFQ (SEQ ID NO: 1011) |
| ORF1 | MPWWYRRRSYNPWRRRNWFRRPRKTIYRRYRRRRRWVRRKPFYKRKIKR |
| | LNIVEWQPKSIRKCRIKGMLCLFQTTEDRLSYNFDMYEESIIPEKLPGGGGFSI |
| | KNISLYALYQEHIHAHNIFTHTNTDRPLARYTGCSLKFYQSKDIDYVVTYSTS |
| | LPLRSSMGMYNSMQPSIHLMQQNKLIVPSKQTQKRRKPYIKKHISPPTQMKS |
| | QWYFQHNIANIPLLMIRTTALTLDNYYIGSRQLSTNVTIHTLNTTYIQNRDW |
| | GDRNKTYYCQTLGTQRYFLYGTHSTAQNINDIKLQELIPLINTQDYVQGFD |
| | WTEKDKHNITTYKEFLTKGAGNPFHAEWITAQNPVIHTANSPTQIEQIYTAS |
| | TTTFQNKKLTDLPTPGYIFITPTVSLRYNPYKDLAERNKCYFVRSKINAHGW |
| | DPEQHQELINSDLPQWLLLFGYPDYIKRTQNFALVDTNYILVDHCPYTNPEK |
| | TPFIPLSTSFIEGRSPYSPSDTHEPDEEDQNRWYPCYQYQQESINSICLSGPGTP |
| | KIPKGITAEAKVKYSFNFKWGGDLPPMSTITNPTDQPTYVVPNNFNETTSLQ |
| | NPTTRPEHFLYSFDERRGQLTEKATKRLLKDWETKETSLLSTEYRFAEPTQT |
| | QAPQEDPSSEEEEESNLFERLLRQRTKQLQLKRRIIQTLKDLQKLE (SEQ ID |
| | NO: 1012) |
| ORF1/1 | MPWWYRRRSYNPWRRRNWFRRPRKTIYRRYRRRRRWPTYVVPNNFNETT |
| | SLQNPTTRPEHFLYSFDERRGQLTEKATKRLLKDWETKETSLLSTEYRFAEP |
| | TQTQAPQEDPSSEEEEESNLFERLLRQRTKQLQLKRRIIQTLKDLQKLE (SEQ |
| | ID NO: 2706) |
| ORF1/2 | MPWWYRRRSYNPWRRRNWFRRPRKTIYRRYRRRRRWNTDSRSQHKHKPH |
| | KRTRPRKKKKRATSSSDSSDSEPSSSSSSAE (SEQ ID NO: 1013) |

TABLE N23
Exemplary Anellovirus nucleic acid
sequence (Alphatorquevirus, Clade 4)

| | |
|---|---|
| Name | Ring20 |
| Genus/Clade | Alphatorquevirus Clade 4 |
| Accession Number | AF122914.3 |
| Full Sequence: 3853 bp | |

(SEQ ID NO: 1014)
Annotations:

| Putative Domain | Base range |
|---|---|
| TATA Box | 86-90 |
| Initiation Element | 104-119 |
| Transcriptional Start Site | 114 |
| 5′ UTR Conserved Domain | 174-244 |
| ORF2 | 354-716 |
| ORF2/2 | 354-712; 2372-2873 |
| ORF2/3 | 354-712; 2565-3075 |
| ORF2t/3 | 354-400; 2565-3075 |
| TAIP | 373-690 |
| ORF1 | 590-2899 |
| ORF1/1 | 590-712; 2372-2899 |
| ORF1/2 | 590-712; 2565-2873 |
| Three open-reading frame region | 2551-2870 |
| Poly(A)-Signal | 3071-3076 |
| GC-rich region | 3733-3853 |

TABLE A23
Exemplary Anellovirus amino acid sequences (Alphatorquevirus)
Ring20 (Alphatorquevirus Clade 4)

| ORF2 | MWTPPRNDQQYLNWQWYTSVLSSHSAMCGCSDAIAHLNHLANLLRAPQN |
|---|---|
| | PPPPDNPRPLPVRALPAPPAAHEAAGDRAPWPMGGGGDAGGAGAGGDADH |
| | GGAAGGPADADLLDAVAAAET (SEQ ID NO: 1015) |
| ORF2/2 | MWTPPRNDQQYLNWQWYTSVLSSHSAMCGCSDAIAHLNHLANLLRAPQN |
| | PPPPDNPRPLPVRALPAPPAAHEAAGDRAPWPMGGGGDAGGAGAGGDADH |
| | GGAAGGPADADLLDAVAAAETLLEIPARNPTPRAIESLEAYKSLTRDTTHRN |
| | LPSMPGTSDVASLARKLFKECNNNQLLLNFFQQAARDPEGTQKCISPTKKRS |
| | KKKARFSPQSSSSEESPRGRTRNRSKAGRKAQRKRRRPSPSSSNSSCSSSESW |
| | ESNSDSCSTKSKKSNKIKISTLPCYQGGGI (SEQ ID NO: 1016) |
| ORF2/3 | MWTPPRNDQQYLNWQWYTSVLSSHSAMCGCSDAIAHLNHLANLLRAPQN |
| | PPPPDNPRPLPVRALPAPPAAHEAAGDRAPWPMGGGGDAGGAGAGGDADH |
| | GGAAGGPADADLLDAVAAAETPQETQKGHRSVSVRPRKGAKRKLAFPPSQ |
| | APPKSPPVGGLGTGAKRVAKLRGRDGDPLPAAQTAAAAAASLGSQTQTPV |
| | QPSPKNPTKSRYQPYLVTKGGGSSILLSGCTINMFPDPKPYCPSSNDWKEEY |
| | EACKYWDRPPRHNLRDPPFYPWAPKNNPCNVSFKLGFK (SEQ ID NO: 1017) |
| ORF2t/3 | MWTPPRNDQQYLNWQWPQETQKGHRSVSVRPRKGAKRKLAFPPSQAPPKS |
| | PPVGGLGTGAKRVAKLRGRDGDPLPAAQTAAAAAASLGSQTQTPVQPSPK |
| | NPTKSRYQPYLVTKGGGSSILLSGCTINMFPDPKPYCPSSNDWKEEYEACKY |
| | WDRPPRHNLRDPPFYPWAPKNNPCNVSFKLGFK (SEQ ID NO: 1018) |
| TAIP | MINNTLTGNGTLLYLAPTLLCAGVPTLSLILIILLICFVPRKIRPRLIIQDPYPCE |
| | HCLLPRLPTRQPVIEHHGLWVVEETPEALAQVETPTMEAPLEDPQTQTC |
| | (SEQ ID NO: 1019) |
| ORF1 | MAYGWWRRRRRRWRRWRRRPWRRRWRTRRRRPARRRGRRRNVRRRRR |
| | GRWRRRYRRWKRKGRRRRKAKIIIRQWQPNYRRRCNIVGYLPILICGGNTV |
| | SRNYATHSDDTNYPGPFGGGMTTDKFSLRILYDEYKRFMNYWTASNEDLD |
| | LCRYLGCTFYFFRHPEVDFIIKINTMPPFLDTTITAPSIHPGLMALDKRARWIP |
| | SLKNRPGKKHYIKIRVGAPKMFTDKWYPQTDLCDMTLLTIYATAADMQYP |
| | FGSPLTDTVVVNSQVLQSMYDETISILPDEKTKRNSLLTSIRSYIPFYNTTQTI |
| | AQLKPFVDAGGHTTGSTTTTWGQLLNTTKFTTTTTTTYTYPGTTNTAVTFIT |
| | ANDTWYRGTAYKDNIKDVPQKAAQLYFQTTQKLLGNTFHGSDETLEYHAG |
| | LYSSIWLSPGRSYFETPGAYTDIKYNPFTDRGEGNMLWIDWLSKKNMKYDK |
| | VQSKCLVADLPLWAAAYGYVEFCSKSTGDTNIHMNARLLIRSPFTDPQLIVH |
| | TDPTKGFVPYSLNFGNGKMPGGSSNVPIRMRAKWYPTLSHQQEVLEALAQS |
| | GPFAYHSDIKKVSLGIKYRFKWIWGGNPVRQQVVRNPCKEPHSSGNRVPRSI |
| | QIVDPRYNSPELTIHAWDFRRGFFGPKAIQRMQQQPTATEFFSAGRKRPRRD |
| | TEVYQSDQEKEQKESSLFPPVKLLRRVPPWEDSEQEQSGSQSSEEETATLSQ |
| | QLKQQLQQQRVLGVKLRLLFNQVQKIQQNQDINPTLLPRGGDLVSFFQAVP |
| | (SEQ ID NO: 1020) |
| ORF1/1 | MAYGWWRRRRRRWRRWRRRPWRRRWRTRRRRPARRRGRRRNVVRNPC |
| | KEPHSSGNRVPRSIQIVDPRYNSPELTIHAWDFRRGFFGPKAIQRMQQQPTAT |
| | EFFSAGRKRPRRDTEVYQSDQEKEQKESSLFPPVKLLRRVPPWEDSEQEQSG |
| | SQSSEEETATLSQQLKQQLQQQRVLGVKLRLLFNQVQKIQQNQDINPTLLPR |
| | GGDLVSFFQAVP (SEQ ID NO: 1021) |
| ORF1/2 | MAYGWWRRRRRRWRRWRRRPWRRRWRTRRRRPARRRGRRRNAARDPEG |
| | TQKCISPTKKRSKKKARFSPQSSSSEESPRGRTRNRSKAGRKAQRKRRRPSPS |
| | SSNSSCSSSESWESNSDSCSTKSKKSNKIKISTLPCYQGGGI (SEQ ID NO: |
| | 1022) |

TABLE N24
Exemplary Anellovirus nucleic acid sequence (Alphatorquevirus)

| | |
|---|---|
| Name | Ring18 |
| Genus/Clade | Alphatorquevirus |
| Accession Number | |
| Full Sequence: 3733 bp | |

(SEQ ID NO: 2507)
Annotations:

| Putative Domain | Base range |
|---|---|
| TATA Box | 67-71 |
| Cap Site | 88-95 |
| Transcriptional Start Site | 95 |
| 5′ UTR Conserved Domain | 155-225 |
| ORF2 | 325-687 |
| TAIP | 347-508 |
| ORF2/2 | 325-683, 2295-2790 |
| ORF2/3 | 325-683, 2488-2962 |
| ORF1 | 561-2771 |
| ORF1/1 | 561-683, 2295-2771 |
| ORF1/2 | 561-683, 2488-2790 |
| Three open-reading frame region | 2447-2771 |
| Poly(A) Signal | 2958-2963 |
| GC-rich region | 3627-3718 |

TABLE A24
Exemplary Anellovirus amino acid sequences for Ring 18 (Alphatorquevirus)

| | |
|---|---|
| ORF1 | MAWGFWGRRRRWRRWRPRRRRWRPRRRRRRRPARRFRARRRVRRRGGR |
| | WRRRYRKWRRGRRRRTHRKKIVIKQWQPNFIRRCYIIGYLPLIFCGENTTA |
| | QNYATHSDDMISKGPYGGGMTTTKFTLRILYDEFTRFMNFWTVSNEDLDL |
| | CRYVGCKLIFFKHPTVDFIVQINTQPPFLDTHLTAASIHPGIMMLSKRRILIPS |
| | LKTRPSRKHRVVVRVGAPRLFQDKWYPQSDLCDTVLLSIFATACDLQYPF |
| | GSPLTDNPCVNFQILGPQYKKHLSISSTMDTTNKQHYDSNLFNQTQLYNTF |
| | QTIAQLKETGQTANISPSWSAVONNMALSNTGENATQSKDTWYKGNTYN |
| | NHITTLAQKTRERFKGATKAALQNYPTIMSTDLYEYHSGIYSSIFLSAGRSY |
| | FETTGAYSDIIYNPFTDKGTGNIIWIDYLTKEDTIFVKNKSKCEIMDMPLWA |
| | ACTGYTEFCAKYTGDSAIIYNARVLIRCPYTEPMLIDHSDPNKGFVPYSFNF |
| | GNGKMPGGSSNVPIRMRAKWYVNIFHQKEVLETIVQSGPFGYKGDIKSAV |
| | LAMKYRFHWKWGGNPISKQVVRNPCSNSSSSAAHRGPRSVQAVDPKYNT |
| | PEVTWHSWDIRRGLFGKAGIKRMQQESDALYIPPGPFKRPRRDTNAQDPEE |
| | QNESSGFRVQQRLPWVHSSQETQSSQEETEAQGSVQDQLLLQLREQRVLR |
| | LQLQQLATQVLKVQAGHGIHPLLSSQA (SEQ ID NO: 1100) |
| ORF1/1 | MAWGFWGRRRRWRRWRSRRRRWRPRRRRRRRPARRFRARRRVVRNPCS |
| | NSSSSAAHRGPRSVQAVDPKYNTPEVTWHSWDIRRGLFGKAGIKRMQQES |
| | DALYIPPGPFKRPRRDTNAQDPEEQNESSGFRVQQRLPWVHSSQETQSSQE |
| | ETEAQGSVQDQLLLQLREQRVLRLQLQQLATQVLKVQAGHGIHPLLSSQA |
| | (SEQ ID NO: 2508) |
| ORF1/2 | MAWGFWGRRRRWRRWRPRRRRWRPRRRRRRRPARRFRARRRDHSRDLA |
| | ETRTPKTQKSKTKAQVSESSSDSRGSTPAKRRKAPKKKRRRRGRYKTNYSS |
| | SSESSEYSDSSSSNSQPKSSKSKQGTAYTPYYLPKRKQSLYV (SEQ ID NO: |
| | 2509) |
| ORF2 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEE (SEQ ID NO: 2510) |
| ORF2/2 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEESSGIPAPTPAPPRPIEDLAAYKQLTRNTIPQ |
| | RSRGTRGTSDEDSLAKQVLKECNKNQMLFTFLQDHSRDLAETRTPKTQKS |
| | KTKAQVSESSSDSRGSTPAKRRKAPKKKRRRRGRYKTNYSSSSESSEYSDS |
| | SSSNSQPKSSKSKQGTAYTPYYLPKRKQSLYV (SEQ ID NO: 2511) |
| ORF2/3 | MWQPPAHNVPGIERNWYESCFRSHAAVCGCGDFVGHLSYLATTLGRPPRP |
| | GPPGGPRTPQIRNLPALPAPQGEPGDRAPWRGASGADAAGGDGGDHGAD |
| | GGDPADVGDDALLAAFELVEETIQETSQRHERPRPRRAKRKLRFQSPAATP |
| | VGPLQPRDAKLPRRNGGAGVGTRPTTPPAPRAASTPTPAPATRNPSPQSPSR |
| | ARHTPPIIFPSVNKVFMFEPHGPKPIQGYNDWLEEYTACKFWDRPPRKLHT |
| | DLPFYPWAPKPQDQVRVSFKLNFQ (SEQ ID NO: 2512) |

Ring19 is an Anellovirus that was isolated from RPE cells. In some embodiments, a method described herein comprises delivering an Anellovector (e.g., an Anellovector having sequence similarity to Ring19) to eye tissue (e.g., to the eye of a subject), for example, to retinal tissue and/or RPE cells.

TABLE N25
Novel Anellovirus nucleic acid sequence (Betatorquevirus)

| | |
|---|---|
| Name | RING 19 |
| Genus/Clade | Betatorquevirus |
| Accession | N/A |
| Full Sequence: 2876 bp | |

(SEQ ID NO: 1023)
Annotations:

| Putative Domain | Base range |
|---|---|
| ORF1 | 283-2250 |
| ORF2 | 59-391 |
| ORF3 | 2277-2462 |
| GC-rich region, or a portion thereof | 2515-2615 |
| 5′ UTR Conserved Domain, or a portion thereof | 1-71 |

TABLE A25
Novel Anellovirus amino acid sequence (Betatorquevirus)
RING 19 (Betatorquevirus)

| | |
|---|---|
| ORF1 | MPWYPRRRYPRRRYRWLRRWRARRPFRPRYRRRYWVRNYSRKRKLFKITT |
| | KEWQPKVIRKTHVKGTYPLFLCTKHRINNNMIQYLDSIAPEHYYGGGGFS |
| | IMQFSLQALYEEFIKAKNWWTNTNCFLPLVRYMGCSFKFYKTEFYDYIVL |
| | IERCYPLACTDEMYLSTQPSIMMLTRKCIFVPCKQNSKGKKPYKKVRVRP |
| | PSQMTTGWHFSQDLANMPLVVLKTSVCSFDRYYTDSTAKSTTIGFKTLNT |
| | QTFRYHDWQEPPTTGYKPQNLLWFYGAENGSPVDPNNTIVSNLIYLGGTG |
| | PYEKGTPIKTNISNYFSEPKLWGNIFHDDYTSGTSPVFVTNKSPSEIKTA |
| | WNTIKDLTVKASGVFTLRTIPLWLPCRYNPFADKATNNKIWLVSIHSDHT |
| | EWKPIDNPLLQRTDLPLWLLVWGWQDWQKKNQQTSQPDINYLTVISSPYI |
| | SCYPKLDYYVLLDEGFWEGHSTYIESITDSDKKHWYPKNRFQIETLNLIA |
| | NTGPGTVKLRENQAAEGHMVYRFNFKLGGCPAPMEKICDPSKQSKYPIPN |
| | NQQQTTSLQSPENPIQTYLYDFDERRGLLTERATKRIKQDHTSEKTVLPF |
| | TGAATDLPILQTTSQEESSSEEEEEQQAEKKLLQLRRKQHRLRERILQLL |
| | DIQNT (SEQ ID NO: 1024) |
| ORF2 | MAERARNLNIIILQMQIQPPIRTFKQTISDWKNLIVHVHDNICNCNKPLE |
| | HTIDTCITNPDELRLNKSTKQQLQKCLGTPEEDTQEDVIDGFADGELDAL |
| | FAQDTEEDTG (SEQ ID NO: 1025) |
| ORF3 | MFGDTHVPNRRMTPEEFEQELIVAGVFRRPPCYYIKDRPTYPYVPKPTDE |
| | KCMVNEDLNFP (SEQ ID NO: 1026) |

TABLE N26
Novel Anellovirus nucleic acid sequence (Betatorquevirus)

| | |
|---|---|
| Name | RING 19 alternate |
| Genus/Clade | Betatorquevirus |
| Accession | N/A |
| Full Sequence: 2876 bp | |

(SEQ ID NO: 1027)
Annotations:

| Putative Domain | Base range |
|---|---|
| ORF1 | 283-2250 |
| ORF2 | 101-391 |
| ORF3 | 2277-2462 |
| GC-rich region, or a portion thereof | 2515-2615 |
| 5′ UTR Conserved Domain, or a portion thereof | 1-71 |

TABLE A26
Novel Anellovirus amino acid sequence (Betatorquevirus)
RING 19 (Betatorquevirus)

| | |
|---|---|
| ORF1 | MPWYPRRRYPRRRYRWLRRWRARRPFRPRYRRRYWVRNYSRKRKLFKITT |
| | KEWQPKVIRKTHVKGTYPLFLCTKHRINNNMIQYLDSIAPEHYYGGGGES |
| | IMQFSLQALYEEFIKAKNWWTNTNCFLPLVRYMGCSFKFYKTEFYDYIVL |
| | IERCYPLACTDEMYLSTQPSIMMLTRKCIFVPCKQNSKGKKPYKKVRVRP |
| | PSQMTTGWHFSQDLANMPLVVLKTSVCSFDRYYTDSTAKSTTIGFKTLNT |
| | QTFRYHDWQEPPTTGYKPQNLLWFYGAENGSPVDPNNTIVSNLIYLGGTG |
| | PYEKGTPIKTNISNYFSEPKLWGNIFHDDYTSGTSPVFVINKSPSEIKTA |
| | WNTIKDLTVKASGVFTLRTIPLWLPCRYNPFADKATNNKIWLVSIHSDHT |
| | EWKPIDNPLLQRTDLPLWLLVWGWQDWQKKNQQTSQPDINYLTVISSPYI |
| | SCYPKLDYYVLLDEGFWEGHSTYIESITDSDKKHWYPKNRFQIETLNLIA |
| | NTGPGTVKLRENQAAEGHMVYRFNFKLGGCPAPMEKICDPSKQSKYPIPN |
| | NQQQTTSLQSPENPIQTYLYDFDERRGLLTERATKRIKQDHTSEKTVLPF |
| | TGAATDLPILQTTSQEESSSEEEEEQQAEKKLLQLRRKQHRLRERILQLL |
| | DIQNT (SEQ ID NO: 1028) |
| ORF2 | MQIQPPIRTFKQTISDWKNLIVHVHDNICNCNKPLEHTIDTCITNPDELR |
| | LNKSTKQQLQKCLGTPEEDTQEDVIDGFADGELDALFAQDTEEDTG |
| | (SEQ ID NO: 1029) |
| ORF3 | MFGDTHVPNRRMTPEEFEQELIVAGVFRRPPCYYIKDRPTYPYVPKPTDE |
| | KCMVNFDLNFP (SEQ ID NO: 1030) |

TABLE N26.1
Novel Anellovirus nucleic acid sequence (Betatorquevirus)

| | |
|---|---|
| Name | RING 19 |
| Genus/Clade | Betatorquevirus |
| Accession | N/A |
| Full Sequence: 2876 bp | |

(SEQ ID NO: 2513)
Annotations:

| Putative Domain | Base range |
|---|---|
| 5′ non-coding region (NCR) | 1-337 |
| 5′ UTR conserved domain | 238-308 |
| ORF2 | 338-624 |
| ORF1 | 520-2487 |
| ORF3 | 2324-2699 |
| 3′ non-coding region (NCR) | 2700-2876 |
| GC-rich region | 2763-2874 |

TABLE N27
Exemplary chicken anemia virus (CAV) nucleic acid sequence

| | |
|---|---|
| Name | CAV isolate Cuxhaven 1 |
| Genus/Clade | Gyrovirus |
| Accession Number | M55918 |
| Full Sequence: 2313 bp | |

(SEQ ID NO: 2771)
Annotations:

| Putative Domain | Base range |
|---|---|
| 5′ UTR | 1-374 |
| Repeat Region | 138-254 |
| CAAT Signal | 255-260 |
| TATA Box | 317-322 |
| VP2 | 374-1024 |
| VP3 (Apoptin) | 480-845 |
| VP1 | 847-2196 |
| 3′ UTR | 2197-2313 |
| GC-Rich Region | 2200-2266 |
| PolyA Signal Sequence | 2281-2286 |

TABLE N28
Alternate exemplary chicken anemia
virus (CAV) nucleic acid sequence

| | |
|---|---|
| Name | CAV isolate Cuxhaven 1 |
| Genus/Clade | Gyrovirus |
| Accession Number | M55918 |
| Full Sequence: 2319 bp | |

(SEQ ID NO: 100)
Annotations:

| Putative Domain | Base range |
|---|---|
| 5′ UTR | 1-379 |
| VP2 | 380-1030 |
| VP3 (Apoptin) | 485-851 |
| VP1 | 853-2202 |
| 3′ UTR | 2203-2319 |

TABLE A28
Exemplary CAV amino acid sequences
CAV

| | |
|---|---|
| VP1 | MARRARRPRGRFYSFRRGRWHHLKRLRRRYKFRHRRRQRYRRRAFRKAFHNPRP |
| | GTYSVRLPNPQSTMTIRFQGVIFLTEGLILPKNSTAGGYADHMYGARVAKISVN |
| | LKEFLLASMNLTYVSKIGGPIAGELIADGSKSQAADNWPNCWLPLDNNVPSATP |
| | SAWWRWALMMMQPTDSCRFFNHPKQMTLQDMGRMFGGWHLFRHIETRFQLLATK |
| | NEGSFSPVASLLSQGEYLTRRDDVKYSSDHQNRWQKGGQPMTGGIAYATGKMRP |
| | DEQQYPAMPPDPPIITATTAQGTQVRCMNSTQAWWSWDTYMSFATLTALGAQWS |
| | FPPGQRSVSRRSFNHHKARGAGDPKGQRWHTLVPLGTETITDSYMSAPASELDT |
| | NFFTLYVAQGTNKSQQYKFGTATYALKEPVMKSDAWAVVRVQSVWQLGNRQRPY |
| | PWDVNWANSTMYWGTQP (SEQ ID NO: 2514) |
| VP2 | MHGNGGQPAAGGSESALSREGQPGPSGAAQGQVISNERSPRRYSTRTINGVQAT |
| | NKFTAVGNPSLQRDPDWYRWNYNHSIAVWLRECSRSHAKICNCGQFRKHWFQEC |
| | AGLEDRSTQASLEEAILRPLRVQGKRAKRKLDYHYSQPTPNRKKAYKTVRWQDE |
| | LADREADFTPSEEDGGTTSSDFDEDINFDIGGDSGIVDELLGRPFTTPAPVRIV |
| | * (SEQ ID NO: 2515) |
| VP3 | MNALQEDTPPGPSTVFRPPTSSRPLETPHCREIRIGIAGITITLSLCGCANARA |
| (Apoptin) | PTLRSATADNSESTGFKNVPDLRTDQPKPPSKKRSCDPSEYRVSELKESLITTT |
| | PSRPRTAKRRIRL* (SEQ ID NO: 2516) |

YYBYFW is an Anellovirus that was isolated from whole blood. In some embodiments, a method described herein comprises delivering an Anellovector (e.g., an Anellovector having sequence similarity to YYBYFW) to blood cells (e.g., to the blood cells of a subject).

TABLE N29
Novel Anellovirus nucleic acid sequence (Betatorquevirus)

| | |
|---|---|
| Name | YYBYFW |
| Genus/Clade | Betatorquevirus |
| Source tissue | Whole blood |
| Accession | N/A |
| Full Sequence: 2876 bp | |

(SEQ ID NO: 200)
Annotations:

| Putative Domain | Base range |
|---|---|
| GC-rich region | 77-194 |
| TATA box | 346-352 |
| Initiation element | 366-381 |
| 5′ UTR Conserved Domain, or a portion thereof | 433-503 |
| ORF2 | 536-817 |
| ORF1 | 688-2616 |
| ORF3 | 2445-2825 |

TABLE A29
Novel Anellovirus amino acid sequence
(Betatorquevirus)
YYBYFW (Betatorquevirus)

| | |
|---|---|
| ORF1 | MPPYYWRQRRPTWRQRNYRRFRRPRRWRIRSLVRRR |
| | WRRRRRVRNKRLFFRRKKLTRLTLKEWQPKKIKKCSIK |
| | GILPAFICGKSRIANNYILFSESIVPVGEPGGGAWSILQIN |
| | LRVLYDQFIKFKNWWTASNQGLPLTRYLGMKVTFYKT |
| | QFVDYIVIPQLCPPFSVTRDNYLNIHPVRMLMNRHKIIIP |
| | QRQKGKKNYIKKWFRPPDLMQNKWYFQQDIVNIPLIVL |
| | HISAASLDQYSIPENQISTNITLWSLNTDFFRNNQFTGYE |
| | DSGYIPKTFGTQNYYLYTEGNGVSRPTKFSQMIPLKNTT |
| | TYTNGKKAATIENWKENSYWGNPFTQNHASPDVPIYY |
| | NNTTPSENNYSTPQTFTPLHELYFQCRYNPEKDTGIGNK |
| | VFFKSNKLKQGEIDTIPDKDEIVITDFPLWLIFYSWISWI |
| | QKSKPIQHIMNDYYFIVITPFIEPKRKSYLFLDTYFVQPIT |
| | NRLTETEKQHWYPQTGYQTDVIFSFAQTGPATPKIDRS |
| | QSIQAQFKYNVKLKWGGCPAPMELITDPSLQDKFPIPNT |
| | IFKTLEIQNPETNKRTLLYDFDERHDGLTDRAAKRIKTE |
| | QIFETSFTGQLPTDPNIRIQTQETTSSSEEESEVSLKEELK |
| | LLKRKNKQLRNKLLRLTSY (SEQ ID NO: 201) |
| ORF2 | LAERARNLMYTFFTDMTHYLKTPQYNGRAMELQLMN |
| | TIIGNHDLCCGCMTPVLHIKEIINTFSYTPCPRTTGDKED |
| | QHGDKETTDALEGLEDGELDRLFAADGEDDEG (SEQ |
| | ID NO: 202) |
| ORF3 | MFGDKATKKLTPIKGKSWEAELQLAKMFKRPLRTYEE |
| | DPPYYPWVVPEPVVNFHLNFKE (SEQ ID NO: 203) |

Ring161 is an Anellovirus that was isolated from retinal tissue and/or RPE cells. In some embodiments, a method described herein comprises delivering an Anellovector (e.g., an Anellovector having sequence similarity to Ring161) to eye tissue (e.g., to the eye of a subject), for example, to retinal tissue and/or RPE cells.

TABLE N30
Novel Anellovirus nucleic acid sequence (Betatorquevirus)

| | |
|---|---|
| Name | Ring161 |
| Genus/Clade | Betatorquevirus |
| Source tissue | Retina (retinal pigment epithelium) |
| Accession | N/A |
| Full Sequence: 2967 bp | |

(SEQ ID NO: 204)
Annotations:

| Putative Domain | Base range |
|---|---|
| 5′ UTR Conserved Domain, or a portion thereof | 1-71 |
| ORF2/3 | 97-393; 2090-2488 |
| ORF2/2 | 97-393; 1963-2247 |
| ORF2 | 97-396 |
| ORF1 | 285-2267 |
| ORF3 | 2069-2488 |
| GC-rich region | 2601-2715 |
| TATA box | 2882-2886 |

TABLE A30
Novel Anellovirus amino acid sequence
(Betatorquevirus)
Ring161 (Betatorquevirus)

| | |
|---|---|
| ORF1 | MPYGWRPWRRRRRRRFWRRRARAPFRRRRYWRYRYR |
| | VRKLRTKPKKLTVKQWQPTKMRRLTVTGKYPLFEGTS |
| | ERIGNNNTQSIDTIAPYLFPSGGLFSITVFTLKGLYELHL |
| | KARNWWSISNCNLPLIKYRGCKITLYRNTDVDFVTVYT |
| | RCGSLTATEELYRSCQPSLLLLNKHKKVLTCKKNNNKR |
| | KISKTIKIPPPALMQNKWYFQKDFANYPLVMLLSAALS |
| | LDRYYTSANSISSTIGFASINTTIFQYSDFKSPNITQPYKP |
| | NDQYWLGTWTNQTVTDFTKVQVQHLILLANTNDYQP |
| | GQPLGPNKTSYKDYFKSKIKWGNPFYAPYFEPEPQKIF |
| | MITSLQKIEEAQPNAYINSLGCTLLPPEKTLLTICRYNPQ |
| | NDRSRHNALYASPVTTAKTKWGATTKPELNTEGLPLW |
| | LLSFGFHDWLLKSEKVQRLDLDYVYVILSEYITPKMEH |
| | YVPVDPNFTQGRSPFEITDHMRPPDVGYWFPKTSYQHT |
| | VFSEIINTGPGTVKLPPRISVEASMKYKFYFTLGGCPPPM |
| | DNVCDPQAQPDFPEPGNIISPTLLQDPEYPIEYYLQAFDE |
| | RRGLLTKKAAKRIKKDYETEQDIFSSTGQTAMDVQLKT |
| | PETTSTEDSSEEEKEPQTLQFQLQRHKRKQRKLRHRILQ |
| | LLKLAKDI (SEQ ID NO: 205) |
| ORF2 | MSDYYKKLTIGPRHAQLNWTNAIVTLHDLQCQCDEPL |
| | CHTIETIVNQEKSLKFSKGLSDKIKSCLTAGDHGAADVV |
| | EDFGDDELERLFAEDGTGDTDTG (SEQ ID NO: 206) |

Ring34 is an Anellovirus that was isolated from muscle tissue. In some embodiments, a method described herein comprises delivering an Anellovector (e.g., an Anellovector having sequence similarity to Ring34) to muscle tissue (e.g., to the muscle of a subject).

TABLE N31
Novel Anellovirus nucleic acid sequence (Betatorquevirus)

| | |
|---|---|
| Name | Ring34 |
| Genus/Clade | Betatorquevirus |
| Source tissue | Muscle |
| Accession | N/A |
| Full Sequence: 2984 bp | |

(SEQ ID NO: 207)
Annotations:

| Putative Domain | Base range |
|---|---|
| 5′ UTR Conserved Domain, or a portion thereof | 1-72 |
| ORF2 | 106-393 |
| ORF1 | 276-2273 |
| ORF3 | 2303-2491 |
| GC-rich region | 2668-2768 |
| Initiation element | 2930-2937 |

TABLE A31
Novel Anellovirus amino acid sequence
(Betatorquevirus)
Ring34 (Betatorquevirus)

| | |
|---|---|
| ORF1 | MPYWYRRPTYYRRRRFWRRKFRGPFRRKLWRRRRRT |
| | YRVRKTKKKLKYLHLKEWQPTRIRKLKITGIMPLYLTT |
| | HDRVSNNMEIYIDSIAPTHVPSGGGFSIQQYTLNGFYEL |
| | FTKGRCWWTQSTKDFPLIRYTGCKIDLYRSQNSDYIFN |
| | YINCYPMRANMETYQSCQPTILQLTKNHKIVRCKKDNN |
| | LKKPYKRIKIHPPAQFQNKWLFQKDMASIPLLMTRCCA |
| | TSLDRWFLASNAQSTTIGFTSLNPETFLLHNFQRPPTAG |
| | YIPRPGVYMFALTNGAHDWSQEKIGDIIYLGNSNHLQP |
| | GITMRELTQSTDTFDTVWTKYFSSSDYWGNIFLAKYLK |
| | QEVRLVVSNLSPSQLKQHYSSFDTAIGKTNFTLRTIPNLI |
| | ECRYNPLQDRGDQNEVFITSIVSDATPIQLPTDPSLKTEN |
| | LPLWLAFMGFIDFQKRQGTAQKVDVNYMVVFHTKYM |
| | SPKNLSWVIPIDNDFLNQHSEYHPDDEVTDYDRENWHP |
| | KTIFQYKTCNEIVACGPGIIKLPQNVSAEAHAKYTFYFK |
| | VGGCAADTKDIKNPEDQPKFPTPINFIQSNSLQNPETPIE |
| | NYLYSFDWRRDVLTPKAAERIAEYCLTEKPFSELTGINI |
| | FETETSPETTSTENQTTQEKEKEALQLLIQQHQLQQRDF |
| | KRRILQLLTQLT (SEQ ID NO: 208) |
| ORF2 | MSRYVPVKNSIKNRNLDWINCIVQVHNLQCSCETPLEH |
| | TVEEILHQEPNLDYKKPTKCPTGTADQLTTEEDVFGEGS |
| | LEALFAENFGEEEDAPTG (SEQ ID NO: 209) |

Ring25 is an Anellovirus that was isolated from retinal tissue and/or RPE cells. In some embodiments, a method described herein comprises delivering an Anellovector (e.g., an Anellovector having sequence similarity to Ring25) to eye tissue (e.g., to the eye of a subject), for example, to retinal tissue and/or RPE cells.

TABLE N32
Novel Anellovirus nucleic acid sequence (Betatorquevirus)

| | |
|---|---|
| Name | Ring25 |
| Genus/Clade | Betatorquevirus |
| Source tissue | Retina |
| Accession | N/A |
| Full Sequence: 2942 bp | |

(SEQ ID NO: 210)
Annotations:

| Putative Domain | Base range |
|---|---|
| 5′ UTR Conserved Domain, or a portion thereof | 1-71 |
| ORF2/3 | 109-407; 2095-2470 |
| ORF2/2 | 109-407; 1974-2250 |
| ORF2 | 109-411 |
| ORF1/1 | 282-407; 1974-2264 |
| ORF1 | 282-2264 |
| ORF1/2 | 282-407; 2095-2250 |
| ORF3 | 2078-2470 |
| GC-rich region | 2570-2703 |
| TATA box | 2856-2862 |
| Initiation element | 2876-2891 |

TABLE A32
Novel Anellovirus amino acid sequence
(Betatorquevirus)
Ring25 (Betatorquevirus)

| | |
|---|---|
| ORF1 | MPYYRRHRWQYNPYTRWRRRYWRRRPRKTFRRRYFP |
| | RRRRQRVRRKFKYLKLKEWQPKTIKKCCIKGMHPIF |
| | ITSAGSISRNYRMYEHSFTPERWPTGGGFSVTKYNL |
| | DGLYEQHSLNRNWWTTSNQTLPLVRYLGCKLKFYQS |
| | WSADYVVNWYNNFPMTATQLLYESCQPSLMMMNKNK |
| | LIVPSKLTQHRKRPYVTLRLPPPAQMSNRWFFARDL |
| | AKVGLAMLTVSCCSLDNYYIGSNQQSNNISFISLNT |
| | YFWQKHNFQQPSIRGYSPYTSGTIEKHIWFTPETTE |
| | VDKLKPTNFILLGTTKENQDGQHISQSNKNTYFTNH |
| | KFWGNPFKHDYFTGASKLYVSTKSWTEITAKMAQID |
| | TDIGAGILTEPSNPLTNICRYAPDRDTGEGNKLYLK |
| | STIRDESGWEPPTDEDLIAEGFPLWILAFGFLDWHK |
| | KLGTATNLDRSWVAVIQSNFISPQLPYYVPLDQYFY |
| | EGTSPYERTDEPHINDEDNKNWFPCTKYQQETLNNL |
| | ASCGPGIYRFGDRKTVEGKLKYQFYFKFGGCAPKMD |
| | TIRDPTKQEVYPIPNCQHSTYSLQNPSMPPESLLWQ |
| | FDVYKDILTSKATKRIGKDFSSKKTVFTDQSAMQPT |
| | ASTAAESDSSEETPDSEEEKTTLLFKYKQQRRKRKW |
| | LEHQLQQLVYQP (SEQ ID NO: 211) |
| ORF2 | MTTKSPSRRELNQLINVCCGAHDLICDCDEPTKHII |
| | LHLFSHGEPYFVTTEQKNNITKCLITDDTAGNTTHT |
| | PAGDDAIGDGDLEKLFAEDTFQEEDAKG (SEQ ID |
| | NO: 212) |

In some embodiments, an anellovector or anelloVLP as described herein is a chimeric anellovector or anelloVLP. In some embodiments, a chimeric anellovector or anelloVLP further comprises one or more elements, polypeptides, or nucleic acids from a virus other than an Anellovirus.

In some embodiments, the chimeric anellovector or anelloVLP comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein).

In some embodiments, the anellovector or anelloVLP comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector or anelloVLP comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In some embodiments, the anellovector or anelloVLP comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In some embodiments, the chimeric ORF1 molecule comprises an ORF1 structural jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 structural arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 structural hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 structural N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 structural C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto.

In some embodiments, the anellovector or anelloVLP comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In some embodiments, the anellovector or anelloVLP comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In some embodiments, the anellovector or anelloVLP comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In some embodiments, the anellovector or anelloVLP comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In some embodiments, the anellovector or anelloVLP comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In some embodiments, the anellovector or anelloVLP comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

In some embodiments, an anellovector comprises a nucleic acid comprising a sequence listed in PCT Application No. PCT/US2018/037379, incorporated herein by reference in its entirety. In some embodiments, an anellovector or anelloVLP comprises a polypeptide comprising a sequence listed in PCT Application No. PCT/US2018/037379, incorporated herein by reference in its entirety.

In some embodiments, an anellovector comprises an Anellovirus genome, e.g., as identified according to the method described in Example 9 of PCT Publication No. WO 2020/123816, incorporated by reference herein in its entirety.

In some embodiments, an anellovector comprises a genetic element comprising a consensus Anellovirus motif, e.g., as shown in Table 19. In some embodiments, an anellovector comprises a genetic element comprising a consensus Anellovirus ORF1 motif, e.g., as shown in Table 19. In some embodiments, an anellovector comprises a genetic element comprising a consensus Anellovirus ORF1/1 motif, e.g., as shown in Table 19. In some embodiments, an anellovector comprises a genetic element comprising a consensus Anellovirus ORF1/2 motif, e.g., as shown in Table 19. In some embodiments, an anellovector comprises a genetic element comprising a consensus Anellovirus ORF2/2 motif, e.g., as shown in Table 19. In some embodiments, an anellovector comprises a genetic element comprising a consensus Anellovirus ORF2/3 motif, e.g., as shown in Table 19. In some embodiments, an anellovector comprises a genetic element comprising a consensus Anellovirus ORF2t/3 motif, e.g., as shown in Table 19. In some embodiments, X, as shown in Table 19, indicates any amino acid. In some embodiments, Z, as shown in Table 19, indicates glutamic acid or glutamine. In some embodiments, B, as shown in Table 19, indicates aspartic acid or asparagine. In some embodiments, J, as shown in Table 19, indicates leucine or isoleucine.

TABLE 19
Consensus motifs in open reading frames
(ORFs) of Anelloviruses

| Con- | | | | |
|---|---|---|---|---|
| sensus | Open | | | SEQ |
| Thresh- | Reading | | | ID |
| old | Frame | Position | Motif | NO: |
| 50 | ORF1 | 79 | LIJRQWQPXXIRRCXIXG | 68 |
| | | | YXPLIXC | |
| 50 | ORF1 | 111 | NYXXHXD | 69 |
| 50 | ORF1 | 135 | FSLXXLYDZ | 70 |
| 50 | ORF1 | 149 | NXWTXSNXDLDLCRYXGC | 71 |
| 50 | ORF1 | 194 | TXPSXHPGXMXLXKHK | 72 |
| 50 | ORF1 | 212 | IPSLXTRPXG | 73 |
| 50 | ORF1 | 228 | RIXPPXLFXDKWYFQXDL | 74 |
| 50 | ORF1 | 250 | LLXIXATA | 75 |
| 50 | ORF1 | 260 | LXXPFXSPXTD | 76 |
| 50 | ORF1 | 448 | YNPXXDKGXGNXIW | 77 |
| 50 | ORF1 | 519 | CPYTZPXL | 78 |
| 50 | ORF1 | 542 | XFGXGXMP | 79 |
| 50 | ORF1 | 569 | HQXEVXEX | 80 |
| 50 | ORF1 | 600 | KYXFXFXWGGNP | 81 |
| 50 | ORF1 | 653 | HSWDXRRG | 82 |
| 50 | ORF1 | 666 | AIKRXQQ | 83 |
| 50 | ORF1 | 750 | XQZQXXLR | 84 |
| 50 | ORF1/1 | 73 | PRXJQXXDP | 85 |
| 50 | ORF1/1 | 91 | HSWDXRRG | 86 |
| 50 | ORF1/1 | 105 | AIKRXQQ | 87 |
| 50 | ORF1/1 | 187 | QZQXXLR | 88 |
| 50 | ORF1/2 | 97 | KXKRRRR | 89 |
| 50 | ORF2/2 | 158 | PIXSLXXYKXXTR | 90 |
| 50 | ORF2/2 | 189 | LAXQLLKECXKN | 91 |
| 50 | ORF2/3 | 39 | HLNXLA | 92 |
| 50 | ORF2/3 | 272 | DRPPR | 93 |
| 50 | ORF2/3 | 281 | DXPFYPWXP | 94 |
| 50 | ORF2/3 | 300 | VXFKLXF | 95 |
| 50 | ORF2t/3 | 4 | WXPPVHBVXGIERXW | 96 |
| 50 | ORF2t/3 | 37 | AKRKLX | 97 |
| 50 | ORF2t/3 | 140 | PSSXDWXXEY | 98 |
| 50 | ORF2t/3 | 156 | DRPPR | 99 |
| 50 | ORF2t/3 | 167 | PFYPW | 2772 |
| 50 | ORF2t/3 | 183 | NVXFKLXF | 101 |
| 50 | ORF1 | 84 | JXXXXWQPXXXXXCXIXG | 102 |
| | | | XXXJWQP | |
| 50 | ORF1 | 149 | NXWXXXNXXXXLXRY | 103 |
| 50 | ORF1 | 448 | YNPXXDXG | 104 |

### ORF1 Molecules

In some embodiments, the anellovector or anelloVLP comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein, e.g., as listed in Table A1-A32), or a functional fragment thereof. In some embodiments, the ORF1 molecule comprises a truncation relative to an Anellovirus ORF1 protein (e.g., an Anellovirus ORF1 protein as described herein, e.g., as listed in Table A1-A32). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 70%, 75%, 80%, 85%, 90%, 95%, 96%, 97%, 98%, 99%, or 100% sequence identity to an Anellovirus ORF1 protein sequence as shown in Table A1-A32. In some embodiments, an ORF1 molecule comprises an amino acid sequence having at least 75%, 80%, 85%, 90%, 95%, 96%, 97%, 98%, or 99% sequence identity to an Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell. In some embodiments, an ORF1 molecule is encoded by an ORF1 nucleic acid. In some embodiments, the ORF1 nucleic acid comprises an antisense strand, which can be directly transcribed to produce mRNA encoding the ORF1 molecule. In some embodiments, the ORF1 nucleic acid comprises a sense strand.

In some embodiments, an ORF1 molecule as described herein comprises an amino acid sequence (e.g., an ORF1 sequence, or an arginine-rich region, jelly-roll domain, HVR, N22, or C-terminal domain sequence) as listed in any of Tables A2, A4, A6, A8, A10, A12, C1-C5, 2, 4, 6, 8, 10, 12, 14, 16, 18, 20-37, or D1-D10 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may encapsidate a nucleic acid molecule (e.g., a genetic element as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer (e.g., comprising a plurality of distinct ORF1 molecules). It is also contemplated that an ORF1 molecule may have replicase activity.

### Anellovirus ORF1 Molecule Domain Structures

Previously, Anellovirus ORF1 molecules have been described as having the following domains, from N-terminus to C-terminus: arginine rich region, jelly-roll region, hypervariable region (HVR), N22 domain, C-terminal domain (CTD). This disclosure, for instance in Examples 17-25 herein, describes the structural analysis that leads to a refined domain structure for Anellovirus ORF1. In particular, domains P1 and P2 have been identified, overlapping with the regions previously referred to as the HVR and N22 domain. In addition, this work has refined the boundaries of the previous domain structures. These refined domain structures are referred to herein as the structural arginine-rich region, the structural jelly-roll region, the structural HVR, the structural N22 domain, and the structural CTD.

### Structural ORF1 Domain-Based Nomenclature for Anellovirus ORF1 Molecules

In an aspect, the present disclosure provides an ORF1 molecule comprising one or more (e.g., 1, 2, 3, 4, or 5) of domains or domain fragments of an Anellovirus ORF1 protein, wherein the boundaries of the domains are defined based on the refined domain structures obtained via the structural analysis described herein, e.g., in Example 17-25. An ORF1 molecule may include, for example, one or more (e.g., 1, 2, 3, 4, or 5) of (e.g., in an N-terminal to C-terminal direction): a structural arginine-rich region, a structural jelly-roll region, a structural hypervariable region (HVR), a structural N22 domain, and a structural C-terminal domain (e.g., as described herein). In some embodiments, the ORF1 molecule comprises a structural arginine-rich region (e.g., as described herein), or an amino acid sequence having at least 75%, 80%, 85%, 90%, 95%, 96%, 97%, 98%, or 99% sequence identity thereto. In some embodiments, the ORF1 molecule comprises a structural jelly-roll region (e.g., as described herein), or an amino acid sequence having at least 75%, 80%, 85%, 90%, 95%, 96%, 97%, 98%, or 99% sequence identity thereto. In some embodiments, the ORF1 molecule comprises a structural hypervariable region (e.g., as described herein), or an amino acid sequence having at least 75%, 80%, 85%, 90%, 95%, 96%, 97%, 98%, or 99% sequence identity thereto. In some embodiments, the ORF1 molecule comprises a structural N22 domain (e.g., as described herein), or an amino acid sequence having at least 75%, 80%, 85%, 90%, 95%, 96%, 97%, 98%, or 99% sequence identity thereto. In some embodiments, the ORF1 molecule comprises a structural C-terminal domain (e.g., as described herein), or an amino acid sequence having at least 75%, 80%, 85%, 90%, 95%, 96%, 97%, 98%, or 99% sequence identity thereto.

### P1/P2 Domain-Based Nomenclature for Anellovirus ORF1 Moelcules

In an aspect, the present disclosure provides an ORF1 molecule comprising one or more (e.g., 1, 2, 3, 4, 5, 6, or 7) domains or domain fragments of an Anellovirus ORF1 protein, wherein the boundaries of the domains are defined based on the structural analysis identifying the P1 and P2 domains of Anellovirus ORF1 proteins (e.g., as described herein, e.g., in Examples 17-25). An ORF1 molecule may include, for example, one or more (e.g., 1, 2, 3, 4, 5, 6, or 7) of (e.g., in an N-terminal to C-terminal direction): a structural arginine-rich region, a jelly-roll B—H strands subdomain, a first P1 domain fragment (e.g., a P1-1 subdomain), a P2 domain, a second P1 domain fragment (e.g., a P1-2 subdomain), a jelly-roll I-strand subdomain, and a structural C-terminal domain (e.g., as described herein). In some embodiments, the ORF1 molecule comprises a structural arginine-rich region (e.g., as described herein), or an amino acid sequence having at least 75%, 80%, 85%, 90%, 95%, 96%, 97%, 98%, or 99% sequence identity thereto. In some embodiments, the ORF1 molecule comprises a jelly-roll B—H strands subdomain (e.g., as described herein), or an amino acid sequence having at least 75%, 80%, 85%, 90%, 95%, 96%, 97%, 98%, or 99% sequence identity thereto. In some embodiments, the ORF1 molecule comprises a P1-1 region (e.g., as described herein), or an amino acid sequence having at least 75%, 80%, 85%, 90%, 95%, 96%, 97%, 98%, or 99% sequence identity thereto. In some embodiments, the ORF1 molecule comprises a P2 domain (e.g., as described herein), or an amino acid sequence having at least 75%, 80%, 85%, 90%, 95%, 96%, 97%, 98%, or 99% sequence identity thereto. In some embodiments, the ORF1 molecule comprises a P1-2 region (e.g., as described herein), or an amino acid sequence having at least 75%, 80%, 85%, 90%, 95%, 96%, 97%, 98%, or 99% sequence identity thereto. In some embodiments, the ORF1 molecule comprises a jelly-roll I strand subdomain (e.g., as described herein), or an amino acid sequence having at least 75%, 80%, 85%, 90%, 95%, 96%, 97%, 98%, or 99% sequence identity thereto. In some embodiments, the ORF1 molecule comprises a structural C-terminal domain (e.g., as described herein), or an amino acid sequence having at least 75%, 80%, 85%, 90%, 95%, 96%, 97%, 98%, or 99% sequence identity thereto.

### Structural Arginine-Rich Region

An ORF1 molecule may, in some embodiments, also include a region comprising the structure or activity of an Anellovirus structural arginine-rich region (e.g., as described herein, e.g., a structural arginine-rich region from an Anellovirus ORF1 protein as described herein). In some embodiments, the region comprises the amino acid sequence of a structural arginine-rich region as described herein, or a sequence having at least 70%, 75% 80%, 85%, 90% 95%, 96%, 97%, 98% and 99% amino acid sequence identity thereto.

A structural arginine rich region generally has at least 70% (e.g., at least about 70, 80, 90, 95, 96, 97, 98, 99, or 100%) sequence identity to a structural arginine-rich region sequence described herein or a sequence of at least about 40 amino acids comprising at least 60%, 70%, or 80% basic residues (e.g., arginine, lysine, or a combination thereof). In some embodiments, an ORF1 molecule as described herein does not comprise a full-length structural arginine-rich region (e.g., as described herein). In some embodiments, an ORF1 molecule as described herein comprises a portion of a structural arginine-rich region, e.g., a C-terminal portion of a structural arginine-rich region (e.g., as described herein). In some embodiments, an ORF1 molecule as described herein does not any substantial portion (e.g., a portion consisting of at least 5, 10, 15, 20, 25, 30, 35, 40, 45, or 50 contiguous amino acids) of a structural arginine-rich region (e.g., as described herein). In some embodiments, the structural arginine-rich region of an ORF1 molecule is replaced by a heterologous sequence, e.g., a sequence from another virus (e.g., a different Anellovirus or a virus other than an Anellovirus, e.g., as described herein), e.g., as described herein.

In some embodiments, the first region of an ORF1 molecule as described herein comprises a structural arginine-rich region. In other embodiments, an ORF1 molecule as described herein does not comprise a structural arginine-rich region or only comprises a portion of a structural arginine-rich region.

### Structural Jelly Roll Region

An ORF1 molecule may, in some embodiments, also include a region comprising the structure or activity of an Anellovirus structural jelly-roll domain or region (e.g., as described herein, e.g., a structural jelly-roll domain or region from an Anellovirus ORF1 protein as described herein). In some embodiments, the region comprises the amino acid sequence of a structural jelly-roll region as described herein, or a sequence having at least 70%, 75% 80%, 85%, 90% 95%, 96%, 97%, 98% and 99% amino acid sequence identity thereto.

In some embodiments, a structural jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the structural jelly-roll domain are part of one or more β-sheets;
(ii) the secondary structure of the structural jelly-roll domain comprises at least four (e.g., at least 4, 5, 6, 7, 8, 9, 10, 11, or 12) β-strands; and/or
(iii) the tertiary structure of the structural jelly-roll domain comprises at least two (e.g., at least 2, 3, or 4) β-sheets; and/or
(iv) the structural jelly-roll domain comprises a ratio of β-sheets to α-helices of at least 2:1, 3:1, 4:1, 5:1, 6:1, 7:1, 8:1, 9:1, or 10:1.

In certain embodiments, a structural jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the structural jelly-roll domain comprises a first β-sheet in antiparallel orientation to a second β-sheet. In certain embodiments, the first β-sheet comprises about four (e.g., 3, 4, 5, or 6) β-strands. In certain embodiments, the second β-sheet comprises about four (e.g., 3, 4, 5, or 6) 0-strands. In embodiments, the first and second β-sheet comprise, in total, about eight (e.g., 6, 7, 8, 9, 10, 11, or 12) β-strands.

In certain embodiments, a structural jelly-roll domain is a component of a capsid protein (e.g., an ORF1 molecule as described herein). In certain embodiments, a structural jelly-roll domain has self-assembly activity. In some embodiments, a polypeptide comprising a structural jelly-roll domain binds to another copy of the polypeptide comprising the structural jelly-roll domain. In some embodiments, a structural jelly-roll domain of a first polypeptide binds to a structural jelly-roll domain of a second copy of the polypeptide.

In some embodiments, an ORF1 molecule as described herein does not comprise a full-length structural jelly-roll region (e.g., as described herein). In some embodiments, an ORF1 molecule as described herein comprises a portion of a structural jelly-roll region, e.g., an N-terminal portion of a structural jelly-roll region (e.g., as described herein). In some embodiments, an ORF1 molecule as described herein does not comprise any substantial portion (e.g., a portion consisting of at least 5, 10, 15, 20, 25, 30, 35, 40, 45, or 50 contiguous amino acids) of a structural jelly-roll region (e.g., as described herein). In some embodiments, the structural jelly-roll region of an ORF1 molecule is replaced by a heterologous sequence, e.g., a sequence from another virus (e.g., a different Anellovirus or a virus other than an Anellovirus, e.g., as described herein), e.g., as described herein.

In some embodiments, the second region of an ORF1 molecule as described herein comprises a structural jelly-roll region.

### Jelly-Roll B—H Strands Subdomain

Structural analysis described herein revealed that the beta strands of the jelly-roll region are in noncontiguous portions of the ORF1 molecule. In particular, a first jelly-roll subdomain comprises beta strands B—H and a second jelly-roll subdomain comprises beta strand I. An ORF1 molecule may thus, in some embodiments, also include a region comprising the structure or activity of the region of an Anellovirus ORF1 protein comprising beta strands B—H of the jelly-roll domain or region (e.g., as described herein). In some embodiments, the region comprises the amino acid sequence of a first portion of a jelly-roll region (e.g., a jelly-roll (B—H) sequence as described herein), or a sequence having at least 70%, 75% 80%, 85%, 90% 95%, 96%, 97%, 98% and 99% amino acid sequence identity thereto.

In some embodiments, the first portion of the jelly-roll domain or region may comprise one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) (3-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the first portion of the jelly-roll domain comprises a first R-sheet in antiparallel orientation to a second β-sheet. In certain embodiments, the first R-sheet comprises about four (e.g., 3, 4, 5, or 6) β-strands. In certain embodiments, the second R-sheet comprises about four (e.g., 3, 4, 5, or 6) β-strands. In embodiments, the first and second R-sheet comprise, in total, about eight (e.g., 6, 7, 8, 9, 10, 11, or 12) β-strands.

In certain embodiments, the jelly-roll B—H strands subdomain is a component of a capsid protein (e.g., an ORF1 molecule as described herein). In certain embodiments, a jelly-roll B—H strands subdomain has self-assembly activity. In some embodiments, a polypeptide comprising a jelly-roll B—H strands subdomain binds to another copy of the polypeptide comprising the jelly-roll B—H strands subdomain. In some embodiments, the jelly-roll B—H strands subdomain of a first polypeptide binds to a jelly-roll B—H strands subdomain of a second copy of the polypeptide.

In some embodiments, an ORF1 molecule as described herein does not comprise a full-length jelly-roll B—H strands subdomain (e.g., as described herein). In some embodiments, an ORF1 molecule as described herein comprises a portion of a jelly-roll B—H strands subdomain, e.g., an N-terminal portion of a jelly-roll B—H strands subdomain (e.g., as described herein). In some embodiments, an ORF1 molecule as described herein does not comprise any substantial portion (e.g., a portion consisting of at least 5, 10, 15, 20, 25, 30, 35, 40, 45, or 50 contiguous amino acids) of a jelly-roll B—H strands subdomain (e.g., as described herein). In some embodiments, the jelly-roll B—H strands subdomain of an ORF1 molecule is replaced by a heterologous sequence, e.g., a sequence from another virus (e.g., a different Anellovirus or a virus other than an Anellovirus, e.g., as described herein), e.g., as described herein.

In some embodiments, the second region of an ORF1 molecule as described herein comprises a jelly-roll B—H strands subdomain.

### Structural N22 Domain (Structural N22 Domain-Based Nomenclature)

An ORF1 molecule may, in some embodiments, also include a region comprising the structure or activity of an Anellovirus structural N22 domain (e.g., as described herein, e.g., an structural N22 domain from an Anellovirus ORF1 protein as described herein). In some embodiments, the region comprises the amino acid sequence of a structural N22 domain as described herein, or a sequence having at least 70%, 75% 80%, 85%, 90% 95%, 96%, 97%, 98% and 99% amino acid sequence identity thereto.

In some embodiments, the third region of an ORF1 molecule as described herein comprises a structural N22 domain.

### Structural Hypervariable Region (HVR)

An ORF1 molecule may, in some embodiments, further comprise a hypervariable region (HVR), e.g., an HVR from an Anellovirus ORF1 protein, e.g., as described herein. In some embodiments, the region comprises the amino acid sequence of a structural HVR as described herein, or a sequence having at least 70%, 75% 80%, 85%, 90% 95%, 96%, 97%, 98% and 99% amino acid sequence identity thereto. In some embodiments, the HVR is positioned between the second region and the third region. In some embodiments, the HVR comprises comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

### P1-1 Subdomain

In some embodiments, an ORF1 molecule comprises a P1 domain. A P1 domain is generally noncontiguous and comprises a P1-1 subdomain and a P1-2 subdomain, which may be separated by a P2 domain. An ORF1 molecule may, in some embodiments, include a region comprising the structure or activity of a first portion of an Anellovirus ORF1 P1 domain, e.g., an Anellovirus ORF1 P1-1 domain (e.g., as described herein, e.g., a P1-1 domain from an Anellovirus ORF1 protein as described herein). In some embodiments, the region comprises the amino acid sequence of a P1-1 domain as described herein, or a sequence having at least 70%, 75% 80%, 85%, 90% 95%, 96%, 97%, 98% and 99% amino acid sequence identity thereto.

In some embodiments, the third region of an ORF1 molecule as described herein comprises a P1-1 domain.

### P2 Domain

An ORF1 molecule may, in some embodiments, also include a region comprising the structure or activity of an Anellovirus ORF1 P2 domain (e.g., as described herein, e.g., a P2 domain from an Anellovirus ORF1 protein as described herein). In some embodiments, the region comprises the amino acid sequence of a P2 domain as described herein, or a sequence having at least 70%, 75% 80%, 85%, 90% 95%, 96%, 97%, 98% and 99% amino acid sequence identity thereto.

In some embodiments, the fourth region of an ORF1 molecule as described herein comprises a P2 domain.

### P1-2 Subdomain

An ORF1 molecule may, in some embodiments, also include a region comprising the structure or activity of a second portion of an Anellovirus ORF1 P1 domain, e.g., an Anellovirus ORF1 P1-2 domain (e.g., as described herein, e.g., a P1-2 domain from an Anellovirus ORF1 protein as described herein). In some embodiments, the region comprises the amino acid sequence of a P1-2 domain as described herein, or a sequence having at least 70%, 75% 80%, 85%, 90% 95%, 96%, 97%, 98% and 99% amino acid sequence identity thereto.

In some embodiments, the fifth region of an ORF1 molecule as described herein comprises a P1-2 domain.

### Jelly-Roll I-Strand Subdomain

An ORF1 molecule may, in some embodiments, also include a region comprising the structure or activity of a second portion of an Anellovirus ORF1 jelly-roll domain, e.g., an Anellovirus ORF1 jelly-roll strand I subdomain (e.g., as described herein, e.g., a jelly-roll strand I subdomain from an Anellovirus ORF1 protein as described herein). In some embodiments, the region comprises the amino acid sequence of a jelly-roll strand I subdomain as described herein, or a sequence having at least 70%, 75% 80%, 85%, 90% 95%, 96%, 97%, 98% and 99% amino acid sequence identity thereto.

In some embodiments, the sixth region of an ORF1 molecule as described herein comprises a jelly-roll strand I subdomain.

### Structural C-Terminal Domain

An ORF1 molecule may also include a region comprising the structure or activity of an Anellovirus structural C-terminal domain (CTD) (e.g., as described herein, e.g., a structural CTD from an Anellovirus ORF1 protein as described herein). In some embodiments, the region comprises the amino acid sequence of a structural C-terminal domain as described herein, or a sequence having at least 70%, 75% 80%, 85%, 90% 95%, 96%, 97%, 98% and 99% amino acid sequence identity thereto. In some embodiments, an ORF1 molecule as described herein does not comprise a full-length structural C-terminal domain (e.g., as described herein). In some embodiments, an ORF1 molecule as described herein comprises a portion of a structural C-terminal domain, e.g., an N-terminal portion of a structural C-terminal domain (e.g., as described herein). In some embodiments, an ORF1 molecule as described herein does not comprise any substantial portion (e.g., a portion consisting of at least 5, 10, 15, 20, 25, 30, 35, 40, 45, or 50 contiguous amino acids) of a structural C-terminal domain (e.g., as described herein). In some embodiments, the structural C-terminal domain of an ORF1 molecule is replaced by a heterologous sequence, e.g., a sequence from another virus (e.g., a different Anellovirus or a virus other than an Anellovirus, e.g., as described herein), e.g., as described herein.

In some embodiments, the fourth region of an ORF1 molecule as described herein comprises a structural C-terminal domain. In other embodiments, the seventh region of an ORF1 molecule as described herein comprises a structural C-terminal domain.

### ORF1 Molecule Structures

In some embodiments, the ORF1 molecule comprises, in N-terminal to C-terminal order, a first region (e.g., comprising a structural arginine-rich region), second region (e.g., comprising a structural jelly-roll region), third region (e.g., comprising a structural N22 domain), and fourth region (e.g., comprising a structural C-terminal domain). In certain embodiments, the ORF1 molecule comprises a structural HVR (e.g., between the third and fourth regions).

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 structural arginine-rich region (e.g., a structural arginine-rich region from an Anellovirus ORF1 protein, e.g., as described herein). In some embodiments, the first region comprises a nuclear localization sigal.

In some embodiments, the second region comprises a structural jelly-roll domain, e.g., the structure or activity of a viral ORF1 structural jelly-roll domain (e.g., a structural jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, at least a portion of the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, the ORF1 molecule comprises, in N-terminal to C-terminal order, a first region (e.g., comprising a structural arginine-rich region), second region (e.g., comprising a jelly-roll B—H strands subdomain), third region (e.g., comprising a first portion of a P1 domain), fourth region (e.g., comprising a P2 domain), fifth region (e.g., comprising a second portion of a P1 domain), sixth region (e.g., comprising a jelly-roll strand I subdomain), and seventh region (e.g., comprising a structural C-terminal domain).

In some embodiments, the first region can bind to a nucleic acid molecule (e.g., DNA). In some embodiments, the basic residues are selected from arginine, histidine, or lysine, or a combination thereof. In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 structural arginine-rich region (e.g., a structural arginine-rich region from an Anellovirus ORF1 protein, e.g., as described herein). In some embodiments, the first region comprises a nuclear localization sigal.

In some embodiments, at least a portion of the seventh region is exposed on the surface of a proteinaceous exterior (e.g., a proteinaceous exterior comprising a multimer of ORF1 molecules, e.g., as described herein).

In some embodiments, one or more of the domains or regions of an ORF1 molecule may be replaced by a heterologous amino acid sequence (e.g., the corresponding region from a heterologous ORF1 molecule). In some embodiments, the heterologous amino acid sequence has a desired functionality, e.g., as described herein.

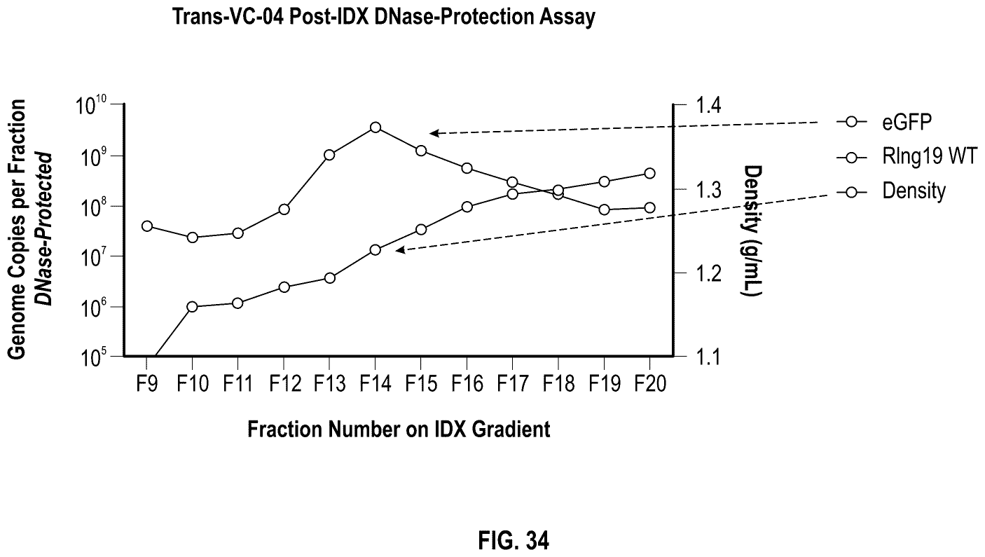

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34 of PCT Publication No. WO2020/123816). In some embodiments, the conserved motifs may show 60, 70, 80, 85, 90, 95, or 100% sequence identity to an ORF1 protein of one or more wild-type Anellovirus clades (e.g., Betatorquevirus). In some embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids. In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule comprises at least one difference (e.g., a mutation, chemical modification, or epigenetic alteration) relative to a wild-type ORF1 protein, e.g., as described herein (e.g., as shown in Table A1-A32).

### Conserved ORF1 Motif in Structural N22 Domain

In some embodiments, a polypeptide (e.g., an ORF1 molecule) described herein comprises the amino acid sequence YNPX²DXGX²N (SEQ ID NO: 829), wherein Xⁿ is a contiguous sequence of any n amino acids. For example, X² indicates a contiguous sequence of any two amino acids. In some embodiments, the YNPX²DXGX²N (SEQ ID NO: 829) is comprised within the structural N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein Xⁿ is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an structural N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

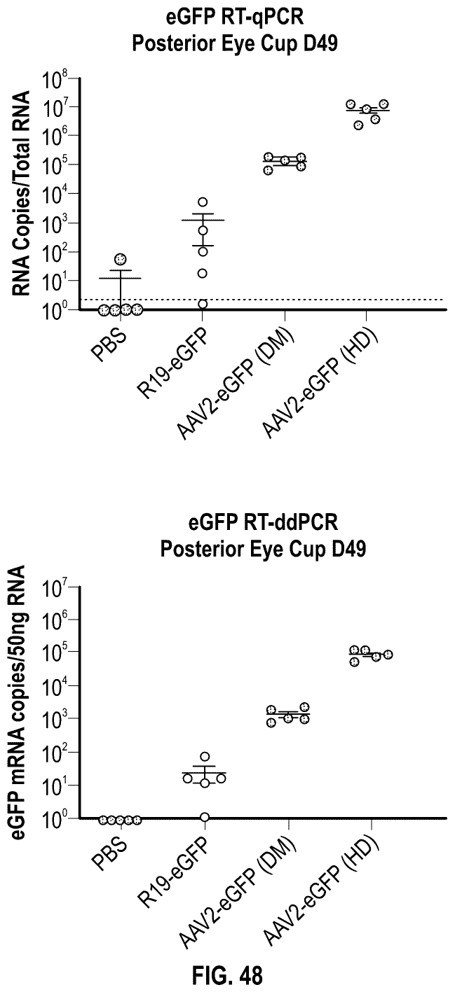

Exemplary YNPX²DXGX²N (SEQ ID NO: 829) motif-flanking secondary structures are described in Example 47 and FIG. 48 of PCT Publication No. WO 2020/123816, incorporated by reference herein in its entirety. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT Publication No. WO 2020/123816. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT Publication No. WO 2020/123816, incorporated by reference herein in its entirety, flanking a YNPX²DXGX²N (SEQ ID NO: 829) motif (e.g., as described herein).

### Conserved Secondary Structural Motif in ORF1 Structural Jelly-Roll Domain

In some embodiments, a polypeptide (e.g., an ORF1 molecule) described herein comprises one or more secondary structural elements comprised by an Anellovirus ORF1 protein (e.g., as described herein). In some emboiments, an ORF1 molecule comprises one or more secondary structural elements comprised by the structural jelly-roll domain of an Anellovius ORF1 protein (e.g., as described herein). Generally, an ORF1 structural jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the structural jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2, 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

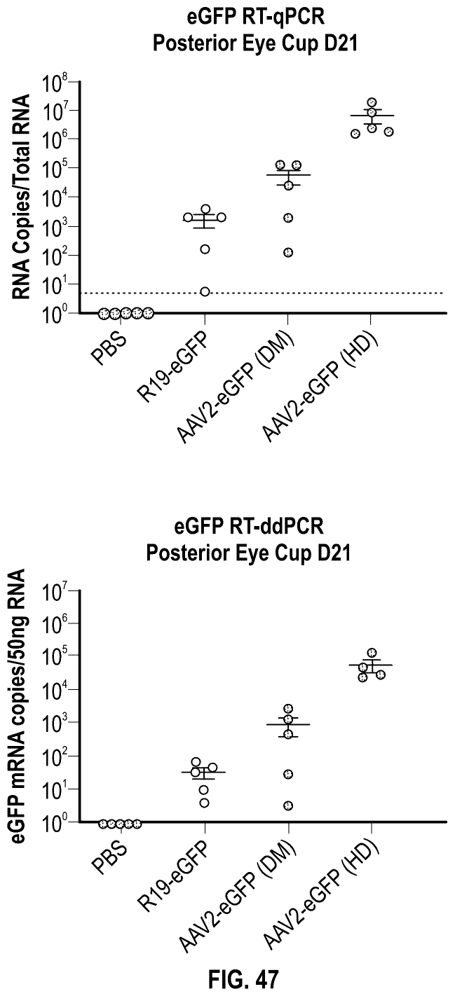

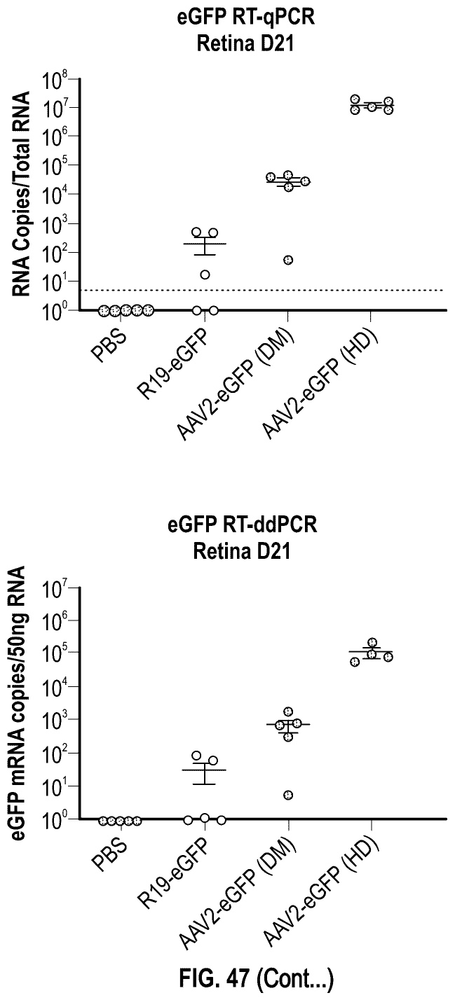

Exemplary structural jelly-roll domain secondary structures are described in Example 47 and FIG. 47 of PCT Publication No. WO 2020/123816, incorporated by reference herein in its entirety. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the structural jelly-roll domain secondary structures shown in FIG. 47 of PCT Publication No. WO 2020/123816, incorporated by reference herein in its entirety.

### Exemplary ORF1 Sequences

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described herein). In some embodiments, an Anelloviridae family vector (e.g., anellovector) described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein. In some embodiments, an anellovector described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein.

In some embodiments, the one or more Anellovirus ORF1 subsequences comprises one or more of a structural arginine (Arg)-rich domain, a structural jelly-roll domain, a hypervariable region (HVR), an structural N22 domain, or a structural C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 molecule comprises one or more of an Arg-rich domain, a structural jelly-roll domain, an structural N22 domain, and a CTD from one Anelloviridae family virus (e.g., Anellovirus), and an HVR from another. In some embodiments, the ORF1 molecule comprises one or more of a structural jelly-roll domain, an HVR, an structural N22 domain, and a CTD from one Anelloviridae family virus (e.g., Anellovirus), and an Arg-rich domain from another. In some embodiments, the ORF1 molecule comprises one or more of an Arg-rich domain, an HVR, an structural N22 domain, and a CTD from one Anellovirus, and a structural jelly-roll domain from another. In some embodiments, the ORF1 molecule comprises one or more of an Arg-rich domain, a structural jelly-roll domain, an HVR, and a CTD from one Anellovirus, and an structural N22 domain from another. In some embodiments, the ORF1 molecule comprises one or more of an Arg-rich domain, a structural jelly-roll domain, an HVR, and an structural N22 domain from one Anellovirus, and a CTD from another.

In some embodiments, the one or more Anellovirus ORF1 subsequences comprises one or more of a structural arginine (Arg)-rich domain, a portion of a jelly-roll domain comprising beta strands B—H (e.g., a jelly-roll B—H strands subdomain as described herein), a first portion of a P1 domain (e.g., a P1-1 sequence), a P2 domain, a second portion of a P1 domain (e.g., a P1-2 sequence), a portion of a jelly-roll domain comprising beta strand I (e.g., a jelly-roll I strand subdomain as described herein), or a structural C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 molecule comprises one or more of a structural arginine-rich domain, a portion of a jelly-roll domain comprising beta strands B—H (e.g., a jelly-roll B—H strands subdomain as described herein), a first portion of a P1 domain (e.g., a P1-1 sequence), a P2 domain, a second portion of a P1 domain (e.g., a P1-2 sequence), a portion of a jelly-roll domain comprising beta strand I (e.g., a jelly-roll I strand subdomain as described herein), and a structural C-terminal domain (CTD) (e.g., as listed herein) from another (e.g., as described herein).

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 domain subsequences, e.g., as described in any of Tables B1-1 to B1-12). In some embodiments, an anellosome described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables B1-1 to B1-12. In some embodiments, an anellosome described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables B1-1 to B1-12.

TABLE B1-1
Ring 2 ORF1 amino acid subsequences
(Betatorquevirus) with structural
HVR and structural N22 domain

| | |
|---|---|
| Structural | MPYYYRRRRYNYRRPRWYGRGWIRRPFRRRFRRK |
| Arginine- | RRVR (SEQ ID NO: 2517) |
| Rich | |
| Region | |
| (1-38) | |
| Structural | PTYTTIPLKQWQPPYKRTCYIKGQDCLIYYSNLR |
| jelly-roll | LGMNSTMYEKSIVPVHWPGGGSFSVSMLTLDALY |
| region | DIHKLCRNWWTSTNQDLPLVRYKGCKITFYQSTF |
| (39-224) | TDYIVRIHTELPANSNKLTYPNTHPLMMMMSKYK |
| | HIIPSRQTRRKKKPYTKIFVKPPPQFENKWYFAT |
| | DLYKIPLLQIHCTACN (SEQ ID NO: 2518) |
| Structural | LQNPFVKPDKLSNNVTLWSLNTISIQNRNMSVDQ |
| hypervariable | GQSWPFKILGTQSFYFYFYTGANLPGDTTQIPVA |
| region | DLLPLTNPRINRPGQSLNEAKITDHITFTEYKNK |
| (225-374) | FTNYWGNPFNKHIQEHLDMILYSLKSPEAIKNEW |
| | TTENMKWNQLNNAG (SEQ ID NO: 2519) |
| Structural | TMALTPFNEPIFTQIQYNPDRDTGEDTQLYLLSN |
| N22 domain | ATGTGWDPPGIPELILEGFPLWLIYWGFADFQKN |
| (375-541) | LKKVTNIDTNYMLVAKTKFTQKPGTFYLVILNDT |
| | FVEGNSPYEKQPLPEDNIKWYPQVQYQLEAQNKL |
| | LQTGPFTPNIQGQLSDNISMFYKFYFKWGGS |
| | (SEQ ID NO: 2520) |
| Structural | PPKAINVENPAHQIQYPIPRNEHETTSLQSPGEA |
| C-terminal | PESILYSFDYRHGNYTTTALSRISQDWALKDTVS |
| domain | KITEPDRQQLLKQALECLQISEETQEKKEKEVQQ |
| (542-666) | LISNLRQQQQLYRERIISLLKDQ (SEQ ID |
| | NO: 2521) |

TABLE B1-2
Ring 2 ORF1 amino acid subsequence
(Betatorquevirus) with P1 and P2 domains

| Structural | MPYYYRRRRYNYRRPRWYGRGWIRRPFRRRFRRKRRV |
|---|---|
| Arg-Rich | R (SEQ ID NO: 216) |
| Region | |
| (1-38) | |
| Jelly-roll | PTYTTIPLKQWQPPYKRTCYIKGQDCLIYYSNLRLGM |
| B-H | NSTMYEKSIVPVHWPGGGSFSVSMLTLDALYDIHKLC |
| strands | RNWWTSTNQDLPLVRYKGCKITFYQSTFTDYIVRIHT |
| subdomain | ELPANSNKLTYPNTHPLMMMMSKYKHIIPSRQTRRKK |
| (39-224) | KPYTKIFVKPPPQFENKWYFATDLYKIPLLQIHCTAC |
| | N (SEQ ID NO: 2518) |
| P1-1 | LQNPFVKPDKLSNNVTLWSL (SEQ ID NO: |
| (225-244) | 2707) |
| P2 | NTISIQNRNMSVDQGQSWPFKILGTQSFYFYFYTGAN |
| (245-385) | LPGDTTQIPVADLLPLTNPRINRPGQSLNEAKITDHI |
| | TFTEYKNKFTNYWGNPFNKHIQEHLDMILYSLKSPEA |
| | IKNEWTTENMKWNQLNNAGTMALTPFNEPI (SEQ |
| | ID NO: 2708) |
| P1-2 | FTQIQYNPDRDTGEDTQLYLLSNATGTGWDPPGIPEL |
| (386-524) | ILEGFPLWLIYWGFADFQKNLKKVTNIDTNYMLVAKT |
| | KFTQKPGTFYLVILNDTFVEGNSPYEKQPLPEDNIKW |
| | YPQVQYQLEAQNKLLQTGPFTPNIQGQL (SEQ ID |
| | NO: 2709) |
| Jelly-roll | SDNISMFYKFYFKWGGS (SEQ ID NO: 2710) |
| I strand | |
| subdomain | |
| (525-541) | |
| Structural | PPKAINVENPAHQIQYPIPRNEHETTSLQSPGEAPES |
| C-terminal | ILYSFDYRHGNYTTTALSRISQDWALKDTVSKITEPD |
| domain | RQQLLKQALECLQISEETQEKKEKEVQQLISNLRQQQ |
| (542-666) | QLYRERIISLLKDQ (SEQ ID NO: 2521) |

TABLE B1-3
Ring 9 ORF1 amino acid subsequence
(Betatorquevirus) with P1 and P2 domains

| | |
|---|---|
| Structural | MPPYWRQKYYRRRYRPFSWRTRRIIQRRKRWRYRK |
| Arg-Rich | PRKTYWRRKLRVRKRFYKR (SEQ ID NO: |
| Region | 2711) |
| (1-54) | |
| Jelly-Roll | KLKKIVLKQFQPKIIRRCTIFGTICLFQGSPERAN |
| B-H | NNYIQTIYSYVPDKEPGGGGWTLITESLSSLWEDW |
| strands | EHLKNVWTQSNAGLPLVRYGGVTLYFYQSAYTDYI |
| subdomain | AQVENCYPMTDTKYTHADSAPNRMLLKKHVIRVPS |
| (55-239) | RETRKKRKPYKRVRVGPPSQMQNKWYFORDICEIP |
| | LIMIAATAVD (SEQ ID NO: 2712) |
| P1-1 | FRYPFCASDCASNNLTLTCL (SEQ ID NO: |
| (240-259) | 2713) |
| P2 | NPLLFQNQDFDHPSDTQGYFPKPGVYLYSTQRSNK |
| (260-369) | PSSSDCIYLGNTKDNQEGKSASSLMTLKTQKITDW |
| | GNPFWHYYIDGSKKIFSYFKPPSQLDSSDFEHMTE |
| | LAEPM (SEQ ID NO: 2714) |
| P1-2 | FIQVRYNPERDTGQGNLIYVTENFRGQHWDPPSSD |
| (370-507) | NLKLDGFPLYDMCWGFIDWIEKVHETENLLTNYCF |
| | CIRSSAFNEKKTVFIPVDHSFLTGFSPYETPVKSS |
| | DQAHWHPQIRFQTKSINDICLTGPGCARSPYGN |
| | (SEQ ID NO: 2715) |
| Jelly-Roll | YMQAKMSYKFHVKWG (SEQ ID NO: 2716) |
| I strand | |
| subdomain | |
| (508-522) | |
| Structural | GCPKTYEKPYDPCSQPNWTIPHNLNETIQIQNPNT |
| C-terminal | CPQTELQEWDWRRDIVTKKAIERIRQHTEPHETLQ |
| domain | ISTGSKHNPPVHRQTSPWTDSETDSEEEKDQTQEI |
| (523-622) | QIQLNKLRKHQQHLKQQLKQYLKPQNIE (SEQ |
| | ID NO: 2717) |

TABLE B1-4
Ring10 ORF1 amino acid subsequence
(Betatorquevirus) with structural
HVR and structural N22 domain

| Structural | MPWWYRRRSYNPWRRRNWFRRPRKTIYRRYRRRRRWV |
|---|---|
| Arg-Rich | RRKPFYKR (SEQ ID NO: 2718) |
| Region | |
| (1-45) | |
| Structural | KIKRLNIVEWQPKSIRKCRIKGMLCLFQTTEDRLSYN |
| jelly-roll | FDMYEESIIPEKLPGGGGFSIKNISLYALYQEHIHAH |
| Domain | NIFTHTNTDRPLARYTGCSLKFYQSKDIDYVVTYSTS |
| (46-229) | LPLRSSMGMYNSMQPSIHLMQQNKLIVPSKQTQKRRK |
| | PYIKKHISPPTQMKSQWYFQHNIANIPLLMIRTTAL |
| | (SEQ ID NO: 2719) |
| Structural | TLDNYYIGSRQLSTNVTIHTLNTTYIQNRDWGDRNKT |
| hyper- | YYCQTLGTQRYFLYGTHSTAQNINDIKLQELIPLTNT |
| variable | QDYVQGFDWTEKDKHNITTYKEFLTKGAGNPFHAEWI |
| domain | TAQNPVIHTANSPTQIEQIYTASTTTFQNKKLTDLPT |
| (230-385) | PGYIFITP (SEQ ID NO: 2720) |
| Structural | TVSLRYNPYKDLAERNKCYFVRSKINAHGWDPEQHQE |
| N22 domain | LINSDLPQWLLLFGYPDYIKRTQNFALVDTNYILVDH |
| (386-543) | CPYTNPEKTPFIPLSTSFIEGRSPYSPSDTHEPDEED |
| | QNRWYPCYQYQQESINSICLSGPGTPKIPKGITAEAK |
| | VKYSFNFKWG (SEQ ID NO: 2721) |
| Structural | GDLPPMSTITNPTDQPTYVVPNNFNETTSLQNPTTRP |
| C-terminal | EHFLYSFDERRGQLTEKATKRLLKDWETKETSLLSTE |
| domain | YRFAEPTQTQAPQEDPSSEEEEESNLFERLLRQRTKQ |
| (544-672) | LQLKRRIIQTLKDLQKLE (SEQ ID NO: 2722) |

TABLE B1-5
Ring10 ORF1 amino acid subsequence
(Betatorquevirus) with P1 and P2 domains

| Structural | MPWWYRRRSYNPWRRRNWFRRPRKTIYRRYRRRRRWVR |
|---|---|
| Arg-Rich | RKPFYKR (SEQ ID NO: 2718) |
| Region | |
| (1-45) | |
| Jelly-roll | KIKRLNIVEWQPKSIRKCRIKGMLCLFQTTEDRLSYNF |
| B-H | DMYEESIIPEKLPGGGGFSIKNISLYALYQEHIHAHNI |
| strands | FTHTNTDRPLARYTGCSLKFYQSKDIDYVVTYSTSLPL |
| subdomain | RSSMGMYNSMQPSIHLMQQNKLIVPSKQTQKRRKPYIK |
| (46-229) | KHISPPTQMKSQWYFQHNIANIPLLMIRTTAL (SEQ |
| | ID NO: 2719) |
| P1-1 | TLDNYYIGSRQLSTNVTIHTL (SEQ ID NO: |
| (230-250aa) | 2723) |
| P2 | NTTYIQNRDWGDRNKTYYCQTLGTQRYFLYGTHSTAQN |
| (251-385) | INDIKLQELIPLTNTQDYVQGFDWTEKDKHNITTYKEF |
| | LTKGAGNPFHAEWITAQNPVIHTANSPTQIEQIYTAST |
| | TTFQNKKLTDLPTPGYIFITP (SEQ ID NO: |
| | 2724) |
| P1-2 | TVSLRYNPYKDLAERNKCYFVRSKINAHGWDPEQHQEL |
| (386-528aa) | INSDLPQWLLLFGYPDYIKRTQNFALVDTNYILVDHCP |
| | YTNPEKTPFIPLSTSFIEGRSPYSPSDTHEPDEEDQNR |
| | WYPCYQYQQESINSICLSGPGTPKIPKGI (SEQ ID |
| | NO: 2725) |
| Jelly-roll | TAEAKVKYSFNFKWG (SEQ ID NO: 2726) |
| I-strand | |
| subdomain | |
| (529-543) | |
| Structural | GDLPPMSTITNPTDQPTYVVPNNFNETTSLQNPTTRPE |
| C-terminal | HFLYSFDERRGQLTEKATKRLLKDWETKETSLLSTEYR |
| domain | FAEPTQTQAPQEDPSSEEEEESNLFERLLRQRTKQLQL |
| (544-672) | KRRIIQTLKDLQKLE (SEQ ID NO: 2722) |

TABLE B1-9
Ring18 ORF1 amino acid subsequence
(Alphatorquevirus) with P1 and P2
domains

| | |
|---|---|
| Structural | MAWGFWGRRRRWRRWRPRRRRWRPRRRRRRRPARRFR |
| Arg-Rich | ARRRVRRRGGRWRRRYRKWRRGRRRRTHRK (SEQ |
| Region | ID NO: 2727) |
| (1-67) | |
| Jelly-roll | KIVIKQWQPNFIRRCYIIGYLPLIFCGENTTAQNYAT |
| B-H | HSDDMISKGPYGGGMTTTKFTLRILYDEFTRFMNFWT |
| strands | VSNEDLDLCRYVGCKLIFFKHPTVDFIVQINTQPPFL |
| subdomain | DTHLTAASIHPGIMMLSKRRILIPSLKTRPSRKHRVV |
| (68-248) | VRVGAPRLFQDKWYPQSDLCDTVLLSIFATACD |
| | (SEQ ID NO: 2728) |
| P1-1 | LQYPFGSPLTDNPCVNFQILGPQYKKHLSIS (SEQ |
| (249-279) | ID NO: 2729) |
| P2 | STMDTTNKQHYDSNLFNQTQLYNTFQTIAQLKETGQT |
| (280-401) | ANISPSWSAVQNNMALSNTGENATQSKDTWYKGNTYN |
| | NHITTLAQKTRERFKGATKAALQNYPTIMSTDLYEYH |
| | SGIYSSIFLSA (SEQ ID NO: 2730) |
| P1-2 | GRSYFETTGAYSDIIYNPFTDKGTGNIIWIDYLTKED |
| (402-555) | TIFVKNKSKCEIMDMPLWAACTGYTEFCAKYTGDSAI |
| | IYNARVLIRCPYTEPMLIDHSDPNKGFVPYSFNFGNG |
| | KMPGGSSNVPIRMRAKWYVNIFHQKEVLETIVQSGPF |
| | GYKGDI (SEQ ID NO: 2731) |
| Jelly-roll | KSAVLAMKYRFHWKWGGNP (SEQ ID NO: 2732) |
| I-strand | |
| subdomain | |
| (556-574) | |
| Structural | ISKQVVRNPCSNSSSSAAHRGPRSVQAVDPKYNTPEV |
| C-terminal | TWHSWDIRRGLFGKAGIKRMQQESDALYIPPGPFKRP |
| domain | RRDTNAQDPEEQNESSGFRVQQRLPWVHSSQETQSSQ |
| (575-736) | EETEAQGSVQDQLLLQLREQRVLRLQLQQLATQVLKV |
| | QAGHGIHPLLSSQA (SEQ ID NO: 2733) |

TABLE B1-11a
Ring19 ORF1 subsequences with structural
HVR and structural N22 domain

| Structural | MPWYPRRRYPRRRYRWLRRWRARRPFRPRYRRRYWVR |
|---|---|
| Arg-Rich | NYSR (SEQ ID NO: 2734) |
| Region | |
| (1-41) | |
| Structural | KRKLFKITTKEWQPKVIRKTHVKGTYPLFLCTKHRIN |
| jelly-roll | NNMIQYLDSIAPEHYYGGGGFSIMQFSLQALYEEFIK |
| region | AKNWWTNTNCFLPLVRYMGCSFKFYKTEFYDYIVLIE |
| (42-228) | RCYPLACTDEMYLSTQPSIMMLTRKCIFVPCKQNSKG |
| | KKPYKKVRVRPPSQMTTGWHFSQDLANMPLVVLKTSV |
| | CS (SEQ ID NO: 2735) |
| Structural | FDRYYTDSTAKSTTIGFKTLNTQTFRYHDWQEPPTTG |
| hyper- | YKPQNLLWFYGAENGSPVDPNNTIVSNLIYLGGTGPY |
| variable | EKGTPIKTNISNYFSEPKLWGNIFHDDYTSGTSPVFV |
| domain | TNKSPSEIKTAWNTIKDLTVKASGVFTLRTIPL |
| (229-372) | (SEQ ID NO: 2736) |
| Structural | WLPCRYNPFADKATNNKIWLVSIHSDHTEWKPIDNPL |
| N22 domain | LQRTDLPLWLLVWGWQDWQKKNQQTSQPDINYLTVIS |
| (373-528) | SPYISCYPKLDYYVLLDEGFWEGHSTYIESITDSDKK |
| | HWYPKNRFQIETLNLIANTGPGTVKLRENQAAEGHMV |
| | YRFNFKLG (SEQ ID NO: 2737) |
| Structural | GCPAPMEKICDPSKQSKYPIPNNQQQTTSLQSPENPI |
| C-terminal | QTYLYDFDERRGLLTERATKRIKQDHTSEKTVLPFTG |
| domain | AATDLPILQTTSQEESSSEEEEEQQAEKKLLQLRRKQ |
| (529-655) | HRLRERILQLLDIQNT (SEQ ID NO: 2738) |

TABLE B1-12
Ring19 ORF1 subsequences with
P1 and P2 domains

| Structural | MPWYPRRRYPRRRYRWLRRWRARRPFRPRYRRRYWV |
|---|---|
| Arg-Rich | RNYSR (SEQ ID NO: 2734) |
| Region | |
| (1-41) | |
| Jelly-roll | KRKLFKITTKEWQPKVIRKTHVKGTYPLFLCTKHRI |
| B-H | NNNMIQYLDSIAPEHYYGGGGFSIMQFSLQALYEEF |
| strands | IKAKNWWTNTNCFLPLVRYMGCSFKFYKTEFYDYIV |
| subdomain | LIERCYPLACTDEMYLSTQPSIMMLTRKCIFVPCKQ |
| (42-228) | NSKGKKPYKKVRVRPPSQMTTGWHFSQDLANMPLVV |
| | LKTSVCS (SEQ ID NO: 2735) |
| P1-1 | FDRYYTDSTAKSTTIGFKT (SEQ ID NO: |
| (229-247aa) | 2739) |
| P2 | LNTQTFRYHDWQEPPTTGYKPQNLLWFYGAENGSPV |
| (248-372) | DPNNTIVSNLIYLGGTGPYEKGTPIKTNISNYFSEP |
| | KLWGNIFHDDYTSGTSPVFVTNKSPSEIKTAWNTIK |
| | DLTVKASGVFTLRTIPL (SEQ ID NO: 2740) |
| P1-2 | WLPCRYNPFADKATNNKIWLVSIHSDHTEWKPIDNP |
| (373-514aa) | LLQRTDLPLWLLVWGWQDWQKKNQQTSQPDINYLTV |
| | ISSPYISCYPKLDYYVLLDEGFWEGHSTYIESITDS |
| | DKKHWYPKNRFQIETLNLIANTGPGTVKLRENQA |
| | (SEQ ID NO: 2741) |
| Jelly-roll | AEGHMVYRFNFKLG (SEQ ID NO: 2742) |
| I-strand | |
| subdomain | |
| (515-528) | |
| Structural | GCPAPMEKICDPSKQSKYPIPNNQQQTTSLQSPENP |
| C-terminal | IQTYLYDFDERRGLLTERATKRIKQDHTSEKTVLPF |
| domain | TGAATDLPILQTTSQEESSSEEEEEQQAEKKLLQLR |
| (529-655) | RKQHRLRERILQLLDIQNT (SEQ ID NO: |
| | 2738) |

### Exemplary ORF1 Variant Construct Sequence Tables

Provided below are exemplary Anellovirus ORF1 mutant and variant sequences. These types of mutants and variants are described in further detail in the sections below.

TABLE B1-3a
Exemplary Ring2 variant constructs suitable for
expression in 293 cells

| #ID | Sequence |
|---|---|
| 1. Ring2 | MLKQWQPPYKRTCYIKGQDCLIYYSNLRLGMNSTMYEKSIVPVHWPGG |
| delARM | GSFSVSMLTLDALYDIHKLCRNWWTSTNQDLPLVRYKGCKITFYQSTF |
| (Δ2-45) | TDYIVRIHTELPANSNKLTYPNTHPLMMMMSKYKHIIPSRQTRRKKKP |
| (7046) | YTKIFVKPPPQFENKWYFATDLYKIPLLQIHCTACNLQNPFVKPDKLS |
| | NNVTLWSLNTISIQNRNMSVDQGQSWPFKILGTQSFYFYFYTGANLPG |
| | DTTQIPVADLLPLTNPRINRPGQSLNEAKITDHITFTEYKNKFTNYWG |
| | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFKWGGSPPKAINVENPAHQIQYPIPRNEHETTSLQSP |
| | GEAPESILYSFDYRHGNYTTTALSRISQDWALKDTVSKITEPDRQQLL |
| | KQALECLQISEETQEKKEKEVQQLISNLRQQQQLYRERIISLLKDQ |
| | (SEQ ID NO: 2522) |
| 2. Ring2 | MPYYYRRRRYNYRRPRWYGRGWIRRPFRRRFRRKRRVRPTYTTIPLKQ |
| delCterm | WQPPYKRTCYIKGQDCLIYYSNLRLGMNSTMYEKSIVPVHWPGGGSFS |
| (Δ611-666) | VSMLTLDALYDIHKLCRNWWTSTNQDLPLVRYKGCKITFYQSTFTDYI |
| (7047) | VRIHTELPANSNKLTYPNTHPLMMMMSKYKHIIPSRQTRRKKKPYTKI |
| | FVKPPPQFENKWYFATDLYKIPLLQIHCTACNLQNPFVKPDKLSNNVT |
| | LWSLNTISIQNRNMSVDQGQSWPFKILGTQSFYFYFYTGANLPGDTTQ |
| | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFTPNIQGQLSDNI |
| | SMFYKFYFKWGGSPPKAINVENPAHQIQYPIPRNEHETTSLOSPGEAP |
| | ESILYSFDYRHGNYTTTALSRISQDWALKDTVSK (SEQ ID NO: |
| | 2523) |
| 3. Ring2 | MPYYYRRRRYNYRRPRWYGRGWIRRPFRRRFRRKRRVRPTYTTIPLKQ |
| delCterm | WQPPYKRTCYIKGQDCLIYYSNLRLGMNSTMYEKSIVPVHWPGGGSFS |
| (Δ538-666) | VSMLTLDALYDIHKLCRNWWTSTNQDLPLVRYKGCKITFYQSTFTDYI |
| (7048) | VRIHTELPANSNKLTYPNTHPLMMMMSKYKHIIPSRQTRRKKKPYTKI |
| | FVKPPPQFENKWYFATDLYKIPLLQIHCTACNLQNPFVKPDKLSNNVT |
| | LWSLNTISIQNRNMSVDQGQSWPFKILGTQSFYFYFYTGANLPGDTTQ |
| | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFTPNIQGQLSDNI |
| | SMFYKFYFK (SEQ ID NO: 2524) |
| 4. Ring2 | MLKQWQPPYKRTCYIKGQDCLIYYSNLRLGMNSTMYEKSIVPVHWPGG |
| delARM | GSFSVSMLTLDALYDIHKLCRNWWTSTNQDLPLVRYKGCKITFYQSTF |
| delCterm | TDYIVRIHTELPANSNKLTYPNTHPLMMMMSKYKHIIPSRQTRRKKKP |
| (Δ2-45 + | YTKIFVKPPPQFENKWYFATDLYKIPLLQIHCTACNLQNPFVKPDKLS |
| Δ611-666) | NNVTLWSLNTISIQNRNMSVDQGQSWPFKILGTQSFYFYFYTGANLPG |
| (7049) | DTTQIPVADLLPLTNPRINRPGQSLNEAKITDHITFTEYKNKFTNYWG |
| | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFKWGGSPPKAINVENPAHQIQYPIPRNEHETTSLOSP |
| | GEAPESILYSFDYRHGNYTTTALSRISQDWALKDTVSK (SEQ ID |
| | NO: 2525) |
| 5. Ring2- | MLKQWQPPYKRTCYIKGQDCLIYYSNLRLGMNSTMYEKSIVPVHWPGG |
| ORF1 | GSFSVSMLTLDALYDIHKLCRNWWTSTNQDLPLVRYKGCKITFYQSTF |
| delARM | TDYIVRIHTELPANSNKLTYPNTHPLMMMMSKYKHIIPSRQTRRKKKP |
| delCterm | YTKIFVKPPPQFENKWYFATDLYKIPLLQIHCTACNLQNPFVKPDKLS |
| (Δ2-45 + | NNVTLWSLNTISIQNRNMSVDQGQSWPFKILGTQSFYFYFYTGANLPG |
| Δ538-666) | DTTQIPVADLLPLTNPRINRPGQSLNEAKITDHITFTEYKNKFTNYWG |
| (7050) | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFK (SEQ ID NO: 2526) |

TABLE B1-3b
Exemplary Ring2 Baculovirus constructs

| #ID | Sequence |
|---|---|
| 6. Ring2 | MLKQWQPPYKRTCYIKGQDCLIYYSNLRLGMNSTMYEKSIVPVHWPGG |
| delARM | GSFSVSMLTLDALYDIHKLCRNWWTSTNQDLPLVRYKGCKITFYQSTF |
| (Δ2-45) | TDYIVRIHTELPANSNKLTYPNTHPLMMMMSKYKHIIPSRQTRRKKKP |
| and Orf2 | YTKIFVKPPPQFENKWYFATDLYKIPLLQIHCTACNLQNPFVKPDKLS |
| (5130) | NNVTLWSLNTISIQNRNMSVDQGQSWPFKILGTQSFYFYFYTGANLPG |
| | DTTQIPVADLLPLTNPRINRPGQSLNEAKITDHITFTEYKNKFTNYWG |
| | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFKWGGSPPKAINVENPAHQIQYPIPRNEHETTSLQSP |
| | GEAPESILYSFDYRHGNYTTTALSRISQDWALKDTVSKITEPDRQQLL |
| | KQALECLQISEETQEKKEKEVQQLISNLRQQQQLYRERIISLLKDQ |
| | (SEQ ID NO: 2527) |
| 7. Ring2 | MLKQWQPPYKRTCYIKGQDCLIYYSNLRLGMNSTMYEKSIVPVHWPGG |
| delARM | GSFSVSMLTLDALYDIHKLCRNWWTSTNQDLPLVRYKGCKITFYQSTF |
| delCterm | TDYIVRIHTELPANSNKLTYPNTHPLMMMMSKYKHIIPSRQTRRKKKP |
| (Δ2-45 + | YTKIFVKPPPQFENKWYFATDLYKIPLLQIHCTACNLQNPFVKPDKLS |
| Δ545-666) | NNVTLWSLNTISIQNRNMSVDQGQSWPFKILGTQSFYFYFYTGANLPG |
| Orf2 | DTTQIPVADLLPLTNPRINRPGQSLNEAKITDHITFTEYKNKFTNYWG |
| (5131) | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFKWGGSPPK (SEQ ID NO: 2528) |
| 8. Ring2 | MLKQWQPPYKRTCYIKGQDCLIYYSNLRLGMNSTMYEKSIVPVHWPGG |
| delARM | GSFSVSMLTLDALYDIHKLCRNWWTSTNQDLPLVRYKGCKITFYQSTF |
| delCterm | TDYIVRIHTELPANSNKLTYPNTHPLMMMMSKYKHIIPSRQTRRKKKP |
| (Δ2-45 + | YTKIFVKPPPQFENKWYFATDLYKIPLLQIHCTACNLQNPFVKPDKLS |
| Δ538-666) | NNVTLWSLNTISIQNRNMSVDQGQSWPFKILGTQSFYFYFYTGANLPG |
| ORF2 | DTTQIPVADLLPLTNPRINRPGQSLNEAKITDHITFTEYKNKFTNYWG |
| (5132) | NPFNKHIQEHLDMILYSLKSPEAIKNEWTTENMKWNQLNNAGTMALTP |
| | FNEPIFTQIQYNPDRDTGEDTQLYLLSNATGTGWDPPGIPELILEGFP |
| | LWLIYWGFADFQKNLKKVTNIDTNYMLVAKTKFTQKPGTFYLVILNDT |
| | FVEGNSPYEKQPLPEDNIKWYPQVQYQLEAQNKLLQTGPFTPNIQGQL |
| | SDNISMFYKFYFK (SEQ ID NO: 2529) |
| 9. Ring2 | MPYYYRRRRYNYRRPRWYGRGWIRRPFRRRFRRKRRVRPTYTTIPLKQ |
| delARM | WQPPYKRTCYIKGQDCLIYYSNLRLGMNSTMYEKSIVPVHWPGGGSFS |
| delCterm | VSMLTLDALYDIHKLCRNWWTSTNQDLPLVRYKGCKITFYQSTFTDYI |
| (Δ2-45 + | VRIHTELPANSNKLTYPNTHPLMMMMSKYKHIIPSRQTRRKKKPYTKI |
| Δ518-666) | FVKPPPQFENKWYFATDLYKIPLLQIHCTACNLQNPFVKPDKLSNNVT |
| ORF2 | LWSLNTISIQNRNMSVDQGQSWPFKILGTQSFYFYFYTGANLPGDTTQ |
| (5136) | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFT (SEQ ID |
| | NO: 2530) |
| 10. Ring2 | MPYYYRRRRYNYRRPRWYGRGWIRRPFRRRFRRKRRVRPTYTTIPLKQ |
| ORF1 | WQPPYKRTCYIKGQDCLIYYSNLRLGMNSTMYEKSIVPVHWPGGGSFS |
| delCterm | VSMLTLDALYDIHKLCRNWWTSTNQDLPLVRYKGCKITFYQSTFTDYI |
| (Δ538-666) | VRIHTELPANSNKLTYPNTHPLMMMMSKYKHIIPSRQTRRKKKPYTKI |
| ORF2 | FVKPPPQFENKWYFATDLYKIPLLQIHCTACNLQNPFVKPDKLSNNVT |
| (5137) | LWSLNTISIQNRNMSVDQGQSWPFKILGTQSFYFYFYTGANLPGDTTQ |
| | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFTPNIQGQLSDNI |
| | SMFYKFYFK (SEQ ID NO: 2531) |
| 11. Ring2 | MPYYYRRRRYNYRRPRWYGRGWIRRPFRRRFRRKRRVRPTYTTIPLKQ |
| ORF1 | WQPPYKRTCYIKGQDCLIYYSNLRLGMNSTMYEKSIVPVHWPGGGSFS |
| delCterm | VSMLTLDALYDIHKLCRNWWTSTNQDLPLVRYKGCKITFYQSTFTDYI |
| (Δ594-666) | VRIHTELPANSNKLTYPNTHPLMMMMSKYKHIIPSRQTRRKKKPYTKI |
| ORF2 | FVKPPPQFENKWYFATDLYKIPLLQIHCTACNLQNPFVKPDKLSNNVT |
| (5138) | LWSLNTISIQNRNMSVDQGQSWPFKILGTQSFYFYFYTGANLPGDTTQ |
| | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFTPNIQGQLSDNI |
| | SMFYKFYFKWGGSPPKAINVENPAHQIQYPIPRNEHETTSLOSPGEAP |
| | ESILYSFDYRHGNYTTT (SEQ ID NO: 2532) |
| 12. Ring 2 | MPYYYRRRRYNYRRPRWYGRGWIRRPFRRRFRRKRRVRPTYTTIPLKQ |
| ORF1 | WQPPYKRTCYIKGQDCLIYYSNLRLGMNSTMYEKSIVPVHWPGGGSFS |
| delCterm | VSMLTLDALYDIHKLCRNWWTSTNQDLPLVRYKGCKITFYQSTFTDYI |
| (Δ630-666) | VRIHTELPANSNKLTYPNTHPLMMMMSKYKHIIPSRQTRRKKKPYTKI |
| ORF2 | FVKPPPQFENKWYFATDLYKIPLLQIHCTACNLQNPFVKPDKLSNNVT |
| (5139) | LWSLNTISIQNRNMSVDQGQSWPFKILGTQSFYFYFYTGANLPGDTTQ |
| | IPVADLLPLTNPRINRPGQSLNEAKITDHITFTEYKNKFTNYWGNPFN |
| | KHIQEHLDMILYSLKSPEAIKNEWTTENMKWNQLNNAGTMALTPFNEP |
| | IFTQIQYNPDRDTGEDTQLYLLSNATGTGWDPPGIPELILEGFPLWLI |
| | YWGFADFQKNLKKVTNIDTNYMLVAKTKFTQKPGTFYLVILNDTFVEG |
| | NSPYEKQPLPEDNIKWYPQVQYQLEAQNKLLQTGPFTPNIQGQLSDNI |
| | SMFYKFYFKWGGSPPKAINVENPAHQIQYPIPRNEHETTSLOSPGEAP |
| | ESILYSFDYRHGNYTTTALSRISQDWALKDTVSKITEPDRQQLLKQAL |
| | ECLQI (SEQ ID NO: 2533) |

TABLE B1-6
293 constructs for Ring10 delARM/C-terminal deletions

| #ID | sequence |
|---|---|
| 13. Ring10 | MIVEWQPKSIRKCRIKGMLCLFQTTEDRLSYNFDMYEESIIPEKLPGGGG |
| delARM | FSIKNISLYALYQEHIHAHNIFTHTNTDRPLARYTGCSLKFYQSKDIDYV |
| (Δ2-51) | VTYSTSLPLRSSMGMYNSMQPSIHLMQQNKLIVPSKQTQKRRKPYIKKHI |
| (7051) | SPPTQMKSQWYFQHNIANIPLLMIRTTALTLDNYYIGSRQLSTNVTIHTL |
| | NTTYIQNRDWGDRNKTYYCQTLGTQRYFLYGTHSTAQNINDIKLQELIPL |
| | TNTQDYVQGFDWTEKDKHNITTYKEFLTKGAGNPFHAEWITAQNPVIHTA |
| | NSPTQIEQIYTASTTTFQNKKLTDLPTPGYIFITPTVSLRYNPYKDLAER |
| | NKCYFVRSKINAHGWDPEQHQELINSDLPQWLLLFGYPDYIKRTQNFALV |
| | DTNYILVDHCPYTNPEKTPFIPLSTSFIEGRSPYSPSDTHEPDEEDQNRW |
| | YPCYQYQQESINSICLSGPGTPKIPKGITAEAKVKYSFNFKWGGDLPPMS |
| | TITNPTDQPTYVVPNNFNETTSLONPTTRPEHFLYSFDERRGQLTEKATK |
| | RLLKDWETKETSLLSTEYRFAEPTQTQAPQEDPSSEEEEESNLFERLLRQ |
| | RTKQLQLKRRIIQTLKDLQKLE (SEQ ID NO: 2534) |
| 14. Ring10- | MPWWYRRRSYNPWRRRNWFRRPRKTIYRRYRRRRRWVRRKPFYKRKIKRL |
| ORF1 | NIVEWQPKSIRKCRIKGMLCLFQTTEDRLSYNFDMYEESIIPEKLPGGGG |
| delCterm- | FSIKNISLYALYQEHIHAHNIFTHTNTDRPLARYTGCSLKFYQSKDIDYV |
| Helix | VTYSTSLPLRSSMGMYNSMQPSIHLMQQNKLIVPSKQTQKRRKPYIKKHI |
| (Δ610-672) | SPPTQMKSQWYFQHNIANIPLLMIRTTALTLDNYYIGSRQLSTNVTIHTL |
| (7052) | NTTYIQNRDWGDRNKTYYCQTLGTQRYFLYGTHSTAQNINDIKLQELIPL |
| | TNTQDYVQGFDWTEKDKHNITTYKEFLTKGAGNPFHAEWITAQNPVIHTA |
| | NSPTQIEQIYTASTTTFQNKKLTDLPTPGYIFITPTVSLRYNPYKDLAER |
| | NKCYFVRSKINAHGWDPEQHQELINSDLPQWLLLFGYPDYIKRTQNFALV |
| | DTNYILVDHCPYTNPEKTPFIPLSTSFIEGRSPYSPSDTHEPDEEDQNRW |
| | YPCYQYQQESINSICLSGPGTPKIPKGITAEAKVKYSFNFKWGGDLPPMS |
| | TITNPTDQPTYVVPNNFNETTSLQNPTTRPEHFLYSFDERRGQLTEKATK |
| | RLLKDWETK (SEQ ID NO: 2535) |
| 15. Ring10- | MPWWYRRRSYNPWRRRNWFRRPRKTIYRRYRRRRRWVRRKPFYKRKIKRL |
| ORF1 | NIVEWQPKSIRKCRIKGMLCLFQTTEDRLSYNFDMYEESIIPEKLPGGGG |
| delCterm- | FSIKNISLYALYQEHIHAHNIFTHTNTDRPLARYTGCSLKFYQSKDIDYV |
| Helix | VTYSTSLPLRSSMGMYNSMQPSIHLMQQNKLIVPSKQTQKRRKPYIKKHI |
| (Δ542-672) | SPPTQMKSQWYFQHNIANIPLLMIRTTALTLDNYYIGSRQLSTNVTIHTL |
| (7053) | NTTYIQNRDWGDRNKTYYCQTLGTQRYFLYGTHSTAQNINDIKLQELIPL |
| | TNTQDYVQGFDWTEKDKHNITTYKEFLTKGAGNPFHAEWITAQNPVIHTA |
| | NSPTQIEQIYTASTTTFQNKKLTDLPTPGYIFITPTVSLRYNPYKDLAER |
| | NKCYFVRSKINAHGWDPEQHQELINSDLPQWLLLFGYPDYIKRTQNFALV |
| | DTNYILVDHCPYTNPEKTPFIPLSTSFIEGRSPYSPSDTHEPDEEDQNRW |
| | YPCYQYQQESINSICLSGPGTPKIPKGITAEAKVKYSFNFK (SEQ ID |
| | NO: 2536) |
| 16. Ring10- | MIVEWQPKSIRKCRIKGMLCLFQTTEDRLSYNFDMYEESIIPEKLPGGGG |
| ORF1 | FSIKNISLYALYQEHIHAHNIFTHTNTDRPLARYTGCSLKFYQSKDIDYV |
| delARM | VTYSTSLPLRSSMGMYNSMQPSIHLMQQNKLIVPSKQTQKRRKPYIKKHI |
| delCterm- | SPPTQMKSQWYFQHNIANIPLLMIRTTALTLDNYYIGSRQLSTNVTIHTL |
| Helix | NTTYIQNRDWGDRNKTYYCQTLGTQRYFLYGTHSTAQNINDIKLQELIPL |
| (Δ2-51 + | TNTQDYVQGFDWTEKDKHNITTYKEFLTKGAGNPFHAEWITAQNPVIHTA |
| 610-672) | NSPTQIEQIYTASTTTFQNKKLTDLPTPGYIFITPTVSLRYNPYKDLAER |
| (7054) | NKCYFVRSKINAHGWDPEQHQELINSDLPQWLLLFGYPDYIKRTQNFALV |
| | DTNYILVDHCPYTNPEKTPFIPLSTSFIEGRSPYSPSDTHEPDEEDQNRW |
| | YPCYQYQQESINSICLSGPGTPKIPKGITAEAKVKYSFNFKWGGDLPPMS |
| | TITNPTDQPTYVVPNNFNETTSLQNPTTRPEHFLYSFDERRGQLTEKATK |
| | RLLKDWETK (SEQ ID NO: 2537) |
| 17. Ring10- | MIVEWQPKSIRKCRIKGMLCLFQTTEDRLSYNFDMYEESIIPEKLPGGGG |
| ORF1 | FSIKNISLYALYQEHIHAHNIFTHTNTDRPLARYTGCSLKFYQSKDIDYV |
| delARM | VTYSTSLPLRSSMGMYNSMQPSIHLMQQNKLIVPSKQTQKRRKPYIKKHI |
| delCterm | SPPTQMKSQWYFQHNIANIPLLMIRTTALTLDNYYIGSRQLSTNVTIHTL |
| (Δ2-51 + | NTTYIQNRDWGDRNKTYYCQTLGTQRYFLYGTHSTAQNINDIKLQELIPL |
| 542-672) | TNTQDYVQGFDWTEKDKHNITTYKEFLTKGAGNPFHAEWITAQNPVIHTA |
| (7055) | NSPTQIEQIYTASTTTFQNKKLTDLPTPGYIFITPTVSLRYNPYKDLAER |
| | NKCYFVRSKINAHGWDPEQHQELINSDLPQWLLLFGYPDYIKRTQNFALV |
| | DTNYILVDHCPYTNPEKTPFIPLSTSFIEGRSPYSPSDTHEPDEEDQNRW |
| | YPCYQYQQESINSICLSGPGTPKIPKGITAEAKVKYSFNFK (SEQ ID |
| | NO: 2538) |
| 18. Ring10 | MKIKRLNIVEWQPKSIRKCRIKGMLCLFQTTEDRLSYNFDMYEESIIPEK |
| ORF1 | LPGGGGFSIKNISLYALYQEHIHAHNIFTHTNTDRPLARYTGCSLKFYQS |
| delARM | KDIDYVVTYSTSLPLRSSMGMYNSMQPSIHLMQQNKLIVPSKQTQKRRKP |
| (Δ2-45) | YIKKHISPPTQMKSQWYFQHNIANIPLLMIRTTALTLDNYYIGSRQLSTN |
| (7092) | VTIHTLNTTYIQNRDWGDRNKTYYCQTLGTQRYFLYGTHSTAQNINDIKL |
| | QELIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPY |
| | KDLAERNKCYFVRSKINAHGWDPEQHQELINSDLPQWLLLFGYPDYIKRT |
| | QNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWGG |
| | DLPPMSTITNPTDQPTYVVPNNFNETTSLQNPTTRPEHFLYSFDERRGQL |
| | TEKATKRLLKDWETKETSLLSTEYRFAEPTQTQAPQEDPSSEEEEESNLF |
| | ERLLRQRTKQLQLKRRIIQTLKDLQKLE (SEQ ID NO: 2539) |
| 19. Ring 10 | MKIKRLNIVEWQPKSIRKCRIKGMLCLFQTTEDRLSYNFDMYEESIIPEK |
| delARM | LPGGGGFSIKNISLYALYQEHIHAHNIFTHTNTDRPLARYTGCSLKFYQS |
| delCterm- | KDIDYVVTYSTSLPLRSSMGMYNSMQPSIHLMQQNKLIVPSKQTQKRRKP |
| 568 | YIKKHISPPTQMKSQWYFQHNIANIPLLMIRTTALTLDNYYIGSRQLSTN |
| (Δ2-45 + | VTIHTLNTTYIQNRDWGDRNKTYYCQTLGTQRYFLYGTHSTAQNINDIKL |
| Δ569-672) | QELIPLTNTQDYVQGFDWTEKDKHNITTYKEFLTKGAGNPFHAEWITAQN |
| (7093) | PVIHTANSPTQIEQIYTASTTTFQNKKLTDLPTPGYIFITPTVSLRYNPY |
| | KDLAERNKCYFVRSKINAHGWDPEQHQELINSDLPQWLLLFGYPDYIKRT |
| | QNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWGG |
| | DLPPMSTITNPTDQPTYVVPNNEN (SEQ ID NO: 2540) |
| 20. Ring 10 | MKIKRLNIVEWQPKSIRKCRIKGMLCLFQTTEDRLSYNFDMYEESIIPEK |
| delARM | LPGGGGFSIKNISLYALYQEHIHAHNIFTHTNTDRPLARYTGCSLKFYQS |
| delCterm | KDIDYVVTYSTSLPLRSSMGMYNSMQPSIHLMQQNKLIVPSKQTQKRRKP |
| (Δ2-45 + | YIKKHISPPTQMKSQWYFQHNIANIPLLMIRTTALTLDNYYIGSRQLSTN |
| Δ566-672) | VTIHTLNTTYIQNRDWGDRNKTYYCQTLGTQRYFLYGTHSTAQNINDIKL |
| (7094) | QELIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPY |
| | KDLAERNKCYFVRSKINAHGWDPEQHQELINSDLPQWLLLFGYPDYIKRT |
| | QNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWGG |
| | DLPPMSTITNPTDQPTYVVPN (SEQ ID NO: 2541) |
| 21. Ring 10 | MKIKRLNIVEWQPKSIRKCRIKGMLCLFQTTEDRLSYNFDMYEESIIPEK |
| delARM | LPGGGGFSIKNISLYALYQEHIHAHNIFTHTNTDRPLARYTGCSLKFYQS |
| delCterm | KDIDYVVTYSTSLPLRSSMGMYNSMQPSIHLMQQNKLIVPSKQTQKRRKP |
| (Δ2-45 + | YIKKHISPPTQMKSQWYFQHNIANIPLLMIRTTALTLDNYYIGSRQLSTN |
| Δ560-672) | VTIHTLNTTYIQNRDWGDRNKTYYCQTLGTQRYFLYGTHSTAQNINDIKL |
| (7095) | QELIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPY |
| | KDLAERNKCYFVRSKINAHGWDPEQHQELINSDLPQWLLLFGYPDYIKRT |
| | QNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWGG |
| | DLPPMSTITNPTDQP (SEQ ID NO: 2542) |
| 22. Ring 10 | MKIKRLNIVEWQPKSIRKCRIKGMLCLFQTTEDRLSYNFDMYEESIIPEK |
| delARM | LPGGGGFSIKNISLYALYQEHIHAHNIFTHTNTDRPLARYTGCSLKFYQS |
| delCterm | KDIDYVVTYSTSLPLRSSMGMYNSMQPSIHLMQQNKLIVPSKQTQKRRKP |
| (Δ2-45 + | YIKKHISPPTQMKSQWYFQHNIANIPLLMIRTTALTLDNYYIGSRQLSTN |
| Δ557-672) | VTIHTLNTTYIQNRDWGDRNKTYYCQTLGTQRYFLYGTHSTAQNINDIKL |
| (7096) | QELIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPY |
| | KDLAERNKCYFVRSKINAHGWDPEQHQELINSDLPQWLLLFGYPDYIKRT |
| | QNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWGG |
| | DLPPMSTITNPT (SEQ ID NO: 2543) |

TABLE B1-7
Ring10 Baculovirus constructs

| #ID | Sequence |
|---|---|
| 23. Ring10 | MKIKRLNIVEWQPKSIRKCRIKGMLCLFQTTEDRLSYNFDMYEESIIPEK |
| delARM | LPGGGGFSIKNISLYALYQEHIHAHNIFTHTNTDRPLARYTGCSLKFYQS |
| (Δ2-45) | KDIDYVVTYSTSLPLRSSMGMYNSMQPSIHLMQQNKLIVPSKQTQKRR |
| (5125) | KPYIKKHISPPTQMKSQWYFQHNIANIPLLMIRTTALTLDNYYIGSRQLS |
| | TNVTIHTLNTTYIQNRDWGDRNKTYYCQTLGTQRYFLYGTHSTAQNIN |
| | DIKLQELIPLTNTQDYVQGFDWTEKDKHNITTYKEFLTKGAGNPFHAE |
| | WITAQNPVIHTANSPTQIEQIYTASTTTFQNKKLTDLPTPGYIFITPTVSLR |
| | YNPYKDLAERNKCYFVRSKINAHGWDPEQHQELINSDLPQWLLLFGYP |
| | DYIKRTQNFALVDTNYILVDHCPYTNPEKTPFIPLSTSFIEGRSPYSPSDT |
| | HEPDEEDQNRWYPCYQYQQESINSICLSGPGTPKIPKGITAEAKVKYSFN |
| | FKWGGDLPPMSTITNPTDQPTYVVPNNFNETTSLQNPTTRPEHFLYSFD |
| | ERRGQLTEKATKRLLKDWETKETSLLSTEYRFAEPTQTQAPQEDPSSEE |
| | EEESNLFERLLRQRTKQLQLKRRIIQTLKDLQKLE (SEQ ID NO: 2544) |
| 24. Ring10 | MIVEWQPKSIRKCRIKGMLCLFQTTEDRLSYNFDMYEESIIPEKLPGGGG |
| delARM | FSIKNISLYALYQEHIHAHNIFTHTNTDRPLARYTGCSLKFYQSKDIDYV |
| (Δ2-51) | VTYSTSLPLRSSMGMYNSMQPSIHLMQQNKLIVPSKQTQKRRKPYIKK |
| (5149) | HISPPTQMKSQWYFQHNIANIPLLMIRTTALTLDNYYIGSRQLSTNVTIH |
| | TLNTTYIQNRDWGDRNKTYYCQTLGTQRYFLYGTHSTAQNINDIKLQE |
| | LIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPYK |
| | DLAERNKCYFVRSKINAHGWDPEQHQELINSDLPQWLLLFGYPDYIKR |
| | TQNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWG |
| | GDLPPMSTITNPTDQPTYVVPNNFNETTSLQNPTTRPEHFLYSFDERRGQ |
| | LTEKATKRLLKDWETKETSLLSTEYRFAEPTQTQAPQEDPSSEEEEESNL |
| | FERLLRQRTKQLQLKRRIIQTLKDLQKLE (SEQ ID NO: 2545) |
| 25. Ring10 | MIVEWQPKSIRKCRIKGMLCLFQTTEDRLSYNFDMYEESIIPEKLPGGGG |
| delARM | FSIKNISLYALYQEHIHAHNIFTHTNTDRPLARYTGCSLKFYQSKDIDYV |
| delCterm | VTYSTSLPLRSSMGMYNSMQPSIHLMQQNKLIVPSKQTQKRRKPYIKK |
| (Δ2- | HISPPTQMKSQWYFQHNIANIPLLMIRTTALTLDNYYIGSRQLSTNVTIH |
| 51 + Δ542- | TLNTTYIQNRDWGDRNKTYYCQTLGTQRYFLYGTHSTAQNINDIKLQE |
| 672)(5150) | LIPLTNTQDYVQGFDWTEKDKHNITTYKEFLTKGAGNPFHAEWITAQN |
| | PVIHTANSPTQIEQIYTASTTTFQNKKLTDLPTPGYIFITPTVSLRYNPYK |
| | DLAERNKCYFVRSKINAHGWDPEQHQELINSDLPQWLLLFGYPDYIKR |
| | TQNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFK |
| | (SEQ ID NO: 2546) |
| 26. Ring10 | MIVEWQPKSIRKCRIKGMLCLFQTTEDRLSYNFDMYEESIIPEKLPGGGG |
| delARM | FSIKNISLYALYQEHIHAHNIFTHTNTDRPLARYTGCSLKFYQSKDIDYV |
| delCterm | VTYSTSLPLRSSMGMYNSMQPSIHLMQQNKLIVPSKQTQKRRKPYIKK |
| (Δ2- | HISPPTQMKSQWYFQHNIANIPLLMIRTTALTLDNYYIGSRQLSTNVTIH |
| 51 + Δ552- | TLNTTYIQNRDWGDRNKTYYCQTLGTQRYFLYGTHSTAQNINDIKLQE |
| 672) | LIPLTNTQDYVQGFDWTEKDKHNITTYKEFLTKGAGNPFHAEWITAQN |
| (5151) | PVIHTANSPTQIEQIYTASTTTFQNKKLTDLPTPGYIFITPTVSLRYNPYK |
| | DLAERNKCYFVRSKINAHGWDPEQHQELINSDLPQWLLLFGYPDYIKR |
| | TQNFALVDTNYILVDHCPYTNPEKTPFIPLSTSFIEGRSPYSPSDTHEPDE |
| | EDQNRWYPCYQYQQESINSICLSGPGTPKIPKGITAEAKVKYSFNFKWG |
| | GDLPPMST (SEQ ID NO: 2547) |

TABLE B1-10
Ring18 delARM/C-terminal deletion constructs

| #ID | Sequence |
|---|---|
| 27. Ring18 | MAWGFWGRRRRWRRWRPRRRRWRPRRRRRRRPARRFRARRRVRRR |
| delCterm | GGRWRRRYRKWRRGRRRRTHRKKIVIKQWQPNFIRRCYIIGYLPLIFCG |
| (Δ651-736) | ENTTAQNYATHSDDMISKGPYGGGMTTTKFTLRILYDEFTRFMNFWTV |
| (5152) | SNEDLDLCRYVGCKLIFFKHPTVDFIVQINTQPPFLDTHLTAASIHPGIM |
| | MLSKRRILIPSLKTRPSRKHRVVVRVGAPRLFQDKWYPQSDLCDTVLLS |
| | IFATACDLQYPFGSPLTDNPCVNFQILGPQYKKHLSISSTMDTTNKQHY |
| | DSNLFNQTQLYNTFQTIAQLKETGQTANISPSWSAVQNNMALSNTGEN |
| | ATQSKDTWYKGNTYNNHITTLAQKTRERFKGATKAALQNYPTIMSTDL |
| | YEYHSGIYSSIFLSAGRSYFETTGAYSDIIYNPFTDKGTGNIIWIDYLTKE |
| | DTIFVKNKSKCEIMDMPLWAACTGYTEFCAKYTGDSAIIYNARVLIRCP |
| | YTEPMLIDHSDPNKGFVPYSFNFGNGKMPGGSSNVPIRMRAKWYVNIF |
| | HQKEVLETIVQSGPFGYKGDIKSAVLAMKYRFHWKWGGNPISKQVVR |
| | NPCSNSSSSAAHRGPRSVQAVDPKYNTPEVTWHSWDIRRGLFGKAGIK |
| | RMQQESDALYIPPGPFKRPRR (SEQ ID NO: 2548) |
| 28. Ring18 | MAWGFWGRRRRWRRWRPRRRRWRPRRRRRRRPARRFRARRRVRRR |
| delCterm | GGRWRRRYRKWRRGRRRRTHRKKIVIKQWQPNFIRRCYIIGYLPLIFCG |
| (Δ582-736) | ENTTAQNYATHSDDMISKGPYGGGMTTTKFTLRILYDEFTRFMNFWTV |
| (5153) | SNEDLDLCRYVGCKLIFFKHPTVDFIVQINTQPPFLDTHLTAASIHPGIM |
| | MLSKRRILIPSLKTRPSRKHRVVVRVGAPRLFQDKWYPQSDLCDTVLLS |
| | IFATACDLQYPFGSPLTDNPCVNFQILGPQYKKHLSISSTMDTTNKQHY |
| | DSNLFNQTQLYNTFQTIAQLKETGQTANISPSWSAVQNNMALSNTGEN |
| | ATQSKDTWYKGNTYNNHITTLAQKTRERFKGATKAALQNYPTIMSTDL |
| | YEYHSGIYSSIFLSAGRSYFETTGAYSDIIYNPFTDKGTGNIIWIDYLTKE |
| | DTIFVKNKSKCEIMDMPLWAACTGYTEFCAKYTGDSAIIYNARVLIRCP |
| | YTEPMLIDHSDPNKGFVPYSFNFGNGKMPGGSSNVPIRMRAKWYVNIF |
| | HQKEVLETIVQSGPFGYKGDIKSAVLAMKYRFHWKWGGNPISKQVVR |
| | (SEQ ID NO: 2549) |
| 29. Ring18 | MVIKQWQPNFIRRCYIIGYLPLIFCGENTTAQNYATHSDDMISKGPYGG |
| delARM | GMTTTKFTLRILYDEFTRFMNFWTVSNEDLDLCRYVGCKLIFFKHPTVD |
| delCterm | FIVQINTQPPFLDTHLTAASIHPGIMMLSKRRILIPSLKTRPSRKHRVVVR |
| (Δ 2-69 + | VGAPRLFQDKWYPQSDLCDTVLLSIFATACDLQYPFGSPLTDNPCVNFQ |
| Δ651-736) | ILGPQYKKHLSISSTMDTTNKQHYDSNLFNQTQLYNTFQTIAQLKETGQ |
| (5154) | TANISPSWSAVQNNMALSNTGENATQSKDTWYKGNTYNNHITTLAQK |
| | TRERFKGATKAALQNYPTIMSTDLYEYHSGIYSSIFLSAGRSYFETTGAY |
| | SDIIYNPFTDKGTGNIIWIDYLTKEDTIFVKNKSKCEIMDMPLWAACTGY |
| | TEFCAKYTGDSAIIYNARVLIRCPYTEPMLIDHSDPNKGFVPYSFNFGNG |
| | KMPGGSSNVPIRMRAKWYVNIFHQKEVLETIVQSGPFGYKGDIKSAVL |
| | AMKYRFHWKWGGNPISKQVVRNPCSNSSSSAAHRGPRSVQAVDPKYN |
| | TPEVTWHSWDIRRGLFGKAGIKRMQQESDALYIPPGPFKRPRR (SEQ ID |
| | NO: 2550) |
| 30. Ring18 | MVIKQWQPNFIRRCYIIGYLPLIFCGENTTAQNYATHSDDMISKGPYGG |
| delARM | GMTTTKFTLRILYDEFTRFMNFWTVSNEDLDLCRYVGCKLIFFKHPTVD |
| delCterm | FIVQINTQPPFLDTHLTAASIHPGIMMLSKRRILIPSLKTRPSRKHRVVVR |
| (Δ 2-69 + | VGAPRLFQDKWYPQSDLCDTVLLSIFATACDLQYPFGSPLTDNPCVNFQ |
| Δ582-736) | ILGPQYKKHLSISSTMDTTNKQHYDSNLFNQTQLYNTFQTIAQLKETGQ |
| (5155) | TANISPSWSAVQNNMALSNTGENATQSKDTWYKGNTYNNHITTLAQK |
| | TRERFKGATKAALQNYPTIMSTDLYEYHSGIYSSIFLSAGRSYFETTGAY |
| | SDIIYNPFTDKGTGNIIWIDYLTKEDTIFVKNKSKCEIMDMPLWAACTGY |
| | TEFCAKYTGDSAIIYNARVLIRCPYTEPMLIDHSDPNKGFVPYSFNFGNG |
| | KMPGGSSNVPIRMRAKWYVNIFHQKEVLETIVQSGPFGYKGDIKSAVL |
| | AMKYRFHWKWGGNPISKQVVR (SEQ ID NO: 2551) |

TABLE B1-13b
Ring19 constructs suitable for expression in 293 cells

| #ID | Sequence |
|---|---|
| 31. Ring19 | MPWYPRRRYPRRRYRWLRRWRARRPFRPRYRRRYWVRNYSRKRKLF |
| delCterm | KITTKEWQPKVIRKTHVKGTYPLFLCTKHRINNNMIQYLDSIAPEHYYG |
| (Δ600-655) | GGGFSIMQFSLQALYEEFIKAKNWWTNTNCFLPLVRYMGCSFKFYKTE |
| (7120) | FYDYIVLIERCYPLACTDEMYLSTQPSIMMLTRKCIFVPCKQNSKGKKP |
| | YKKVRVRPPSQMTTGWHFSQDLANMPLVVLKTSVCSFDRYYTDSTAK |
| | STTIGFKTLNTQTFRYHDWQEPPTTGYKPQNLLWFYGAENGSPVDPNN |
| | TIVSNLIYLGGTGPYEKGTPIKTNISNYFSEPKLWGNIFHDDYTSGTSPVF |
| | VTNKSPSEIKTAWNTIKDLTVKASGVFTLRTIPLWLPCRYNPFADKATN |
| | NKIWLVSIHSDHTEWKPIDNPLLQRTDLPLWLLVWGWQDWQKKNQQT |
| | SQPDINYLTVISSPYISCYPKLDYYVLLDEGFWEGHSTYIESITDSDKKH |
| | WYPKNRFQIETLNLIANTGPGTVKLRENQAAEGHMVYRFNFKLGGCPA |
| | PMEKICDPSKQSKYPIPNNQQQTTSLQSPENPIQTYLYDFDERRGLLTER |
| | ATKRIKQDHTSEKTVLP (SEQ ID NO: 2552) |
| 32. Ring19 | MPWYPRRRYPRRRYRWLRRWRARRPFRPRYRRRYWVRNYSRKRKLF |
| delCterm | KITTKEWQPKVIRKTHVKGTYPLFLCTKHRINNNMIQYLDSIAPEHYYG |
| (Δ583-655) | GGGFSIMQFSLQALYEEFIKAKNWWTNTNCFLPLVRYMGCSFKFYKTE |
| (7121) | FYDYIVLIERCYPLACTDEMYLSTQPSIMMLTRKCIFVPCKQNSKGKKP |
| | YKKVRVRPPSQMTTGWHFSQDLANMPLVVLKTSVCSFDRYYTDSTAK |
| | STTIGFKTLNTQTFRYHDWQEPPTTGYKPQNLLWFYGAENGSPVDPNN |
| | TIVSNLIYLGGTGPYEKGTPIKTNISNYFSEPKLWGNIFHDDYTSGTSPVF |
| | VTNKSPSEIKTAWNTIKDLTVKASGVFTLRTIPLWLPCRYNPFADKATN |
| | NKIWLVSIHSDHTEWKPIDNPLLQRTDLPLWLLVWGWQDWQKKNQQT |
| | SQPDINYLTVISSPYISCYPKLDYYVLLDEGFWEGHSTYIESITDSDKKH |
| | WYPKNRFQIETLNLIANTGPGTVKLRENQAAEGHMVYRFNFKLGGCPA |
| | PMEKICDPSKQSKYPIPNNQQQTTSLQSPENPIQTYLYDFDERRGLLTER |
| | (SEQ ID NO: 2553) |

TABLE B1-14
Ring19 delARM/C-terminal deletion: Baculovirus constructs

| #ID | Sequence |
|---|---|
| 33. Ring19 | MTKEWQPKVIRKTHVKGTYPLFLCTKHRINNNMIQYLDSIAPEHYYGG |
| delARM | GGFSIMQFSLQALYEEFIKAKNWWTNTNCFLPLVRYMGCSFKFYKTEF |
| (Δ2-49) | YDYIVLIERCYPLACTDEMYLSTQPSIMMLTRKCIFVPCKQNSKGKKPY |
| (5146) | KKVRVRPPSQMTTGWHFSQDLANMPLVVLKTSVCSFDRYYTDSTAKS |
| | TTIGFKTLNTQTFRYHDWQEPPTTGYKPQNLLWFYGAENGSPVDPNNTI |
| | VSNLIYLGGTGPYEKGTPIKTNISNYFSEPKLWGNIFHDDYTSGTSPVFV |
| | TNKSPSEIKTAWNTIKDLTVKASGVFTLRTIPLWLPCRYNPFADKATNN |
| | KIWLVSIHSDHTEWKPIDNPLLQRTDLPLWLLVWGWQDWQKKNQQTS |
| | QPDINYLTVISSPYISCYPKLDYYVLLDEGFWEGHSTYIESITDSDKKHW |
| | YPKNRFQIETLNLIANTGPGTVKLRENQAAEGHMVYRFNFKLGGCPAP |
| | MEKICDPSKQSKYPIPNNQQQTTSLQSPENPIQTYLYDFDERRGLLTERA |
| | TKRIKQDHTSEKTVLPFTGAATDLPILQTTSQEESSSEEEEEQQAEKKLL |
| | QLRRKQHRLRERILQLLDIQNT (SEQ ID NO: 2554) |
| 34. Ring19 | MTKEWQPKVIRKTHVKGTYPLFLCTKHRINNNMIQYLDSIAPEHYYGG |
| delARM | GGFSIMQFSLQALYEEFIKAKNWWTNTNCFLPLVRYMGCSFKFYKTEF |
| dCterm | YDYIVLIERCYPLACTDEMYLSTQPSIMMLTRKCIFVPCKQNSKGKKPY |
| (Δ2-49 + | KKVRVRPPSQMTTGWHFSQDLANMPLVVLKTSVCSFDRYYTDSTAKS |
| Δ537-655) | TTIGFKTLNTQTFRYHDWQEPPTTGYKPQNLLWFYGAENGSPVDPNNTI |
| (5147) | VSNLIYLGGTGPYEKGTPIKTNISNYFSEPKLWGNIFHDDYTSGTSPVFV |
| | TNKSPSEIKTAWNTIKDLTVKASGVFTLRTIPLWLPCRYNPFADKATNN |
| | KIWLVSIHSDHTEWKPIDNPLLQRTDLPLWLLVWGWQDWQKKNQQTS |
| | QPDINYLTVISSPYISCYPKLDYYVLLDEGFWEGHSTYIESITDSDKKHW |
| | YPKNRFQIETLNLIANTGPGTVKLRENQAAEGHMVYRFNFKLGGCPAP |
| | MEK (SEQ ID NO: 2555) |
| 35. Ring19 | MTKEWQPKVIRKTHVKGTYPLFLCTKHRINNNMIQYLDSIAPEHYYGG |
| delARM | GGFSIMQFSLQALYEEFIKAKNWWTNTNCFLPLVRYMGCSFKFYKTEF |
| delCterm | YDYIVLIERCYPLACTDEMYLSTQPSIMMLTRKCIFVPCKQNSKGKKPY |
| (Δ2-49 + | KKVRVRPPSQMTTGWHFSQDLANMPLVVLKTSVCSFDRYYTDSTAKS |
| Δ527-655) | TTIGFKTLNTQTFRYHDWQEPPTTGYKPQNLLWFYGAENGSPVDPNNTI |
| (5148) | VSNLIYLGGTGPYEKGTPIKTNISNYFSEPKLWGNIFHDDYTSGTSPVFV |
| | TNKSPSEIKTAWNTIKDLTVKASGVFTLRTIPLWLPCRYNPFADKATNN |
| | KIWLVSIHSDHTEWKPIDNPLLQRTDLPLWLLVWGWQDWQKKNQQTS |
| | QPDINYLTVISSPYISCYPKLDYYVLLDEGFWEGHSTYIESITDSDKKHW |
| | YPKNRFQIETLNLIANTGPGTVKLRENQAAEGHMVYRFNFK (SEQ ID |
| | NO: 2556) |

TABLE B2-1
Ring2 cysteine deletion

| | |
|---|---|
| 1. Ring2-ORF1 | MLKQWQPPYKRTSYIKGQDSLIYYSNLRLGMNSTMYEKSIVPV |
| delARM (Δ2-45) + | HWPGGGSFSVSMLTLDALYDIHKLSRNWWTSTNQDLPLVRYK |
| All Cysteine to | GSKITFYQSTFTDYIVRIHTELPANSNKLTYPNTHPLMMMMSK |
| Serine (C57S, C64S, | YKHIIPSRQTRRKKKPYTKIFVKPPPQFENKWYFATDLYKIPLL |
| C112S, C131S, | QIHSTASNLQNPFVKPDKLSNNVTLWSLNTISIQNRNMSVDQG |
| C220S, C223S, | QSWPFKILGTQSFYFYFYTGANLPGDTTQIPVADLLPLTNPRIN |
| C626S) | RPGQSLNEAKITDHITFTEYKNKFTNYWGNPFNKHIQEHLDMIL |
| (SB-7078/pRTx- | YSLKSPEAIKNEWTTENMKWNQLNNAGTMALTPFNEPIFTQIQ |
| 2683) | YNPDRDTGEDTQLYLLSNATGTGWDPPGIPELILEGFPLWLIY |
| | WGFADFQKNLKKVTNIDTNYMLVAKTKFTQKPGTFYLVILND |
| | TFVEGNSPYEKQPLPEDNIKWYPQVQYQLEAQNKLLQTGPFTP |
| | NIQGQLSDNISMFYKFYFKWGGSPPKAINVENPAHQIQYPIPRN |
| | EHETTSLQSPGEAPESILYSFDYRHGNYTTTALSRISQDWALKD |
| | TVSKITEPDRQQLLKQALESLQISEETQEKKEKEVQQLISNLRQ |
| | QQQLYRERIISLLKDQ (SEQ ID NO: 2557) |

TABLE B2-2
Ring10 cysteine deletion with surface moiety grafting

| #ID | Sequence |
|---|---|
| 2. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to | LKFYQSKDIDYVVTYSTSLPLRSSMGMYNSMQPSIHLMQQNK |
| Serine (C63S, C70S, | LIVPSKQTQKRRKPYIKKHISPPTQMKSQWYFQHNIANIPLLMI |
| C137S, C269S, | RTTALTLDNYYIGSRQLSTNVTIHTLNTTYIQNRDWGDRNKTY |
| C403S, C460S, | YSQTLGTQRYFLYGTHSTAQNINDIKLQELIPLINTQDYVQGF |
| C503S, C515S) | DWTEKDKHNITTYKEFLTKGAGNPFHAEWITAQNPVIHTANSP |
| (SB-7079/pRTx- | TQIEQIYTASTTTFQNKKLTDLPTPGYIFITPTVSLRYNPYKDLA |
| 2684) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITAE |
| | AKVKYSFNFKWGGDLPPMSTITNPTDQPTYVVPNNFNETTSLQ |
| | NPTTRPEHFLYSFDERRGQLTEKATKRLLKDWETKETSLLSTE |
| | YRFAEPTQTQAPQEDPSSEEEEESNLFERLLRQRTKQLQLKRRII |
| | QTLKDLQKLE (SEQ ID NO: 2558) |
| 3. Ring10-ORF1 | MIVEWQPKSIRKARIKGMLSLFQTTEDRLSYNFDMYEESIIPEK |
| delARM (Δ2-51) + | LPGGGGFSIKNISLYALYQEHIHAHNIFTHTNTDRPLARYTGAS |
| Buried Cysteines to | LKFYQSKDIDYVVTYSTSLPLRSSMGMYNSMQPSIHLMQQNK |
| Alanines (C63A, | LIVPSKQTQKRRKPYIKKHISPPTQMKSQWYFQHNIANIPLLMI |
| C137A, C269A, | RTTALTLDNYYIGSRQLSTNVTIHTLNTTYIQNRDWGDRNKTY |
| C403A, C460A, | YAQTLGTQRYFLYGTHSTAQNINDIKLQELIPLTNTQDYVQGF |
| C503A, C515A) + | DWTEKDKHNITTYKEFLTKGAGNPFHAEWITAQNPVIHTANSP |
| Exposed Cysteines to | TQIEQIYTASTTTFQNKKLTDLPTPGYIFITPTVSLRYNPYKDLA |
| Serine (C70S) | ERNKAYFVRSKINAHGWDPEQHQELINSDLPQWLLLFGYPDYI |
| (SB-7082/pRTx- | KRTQNFALVDTNYILVDHAPYTNPEKTPFIPLSTSFIEGRSPYSP |
| 2687) | SDTHEPDEEDQNRWYPAYQYQQESINSIALSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2559) |

TABLE B2-3
Ring10 delARM grafting with free cysteine on P2 domain

| #ID | Sequence |
|---|---|
| 4. Ring10-ORF1 | MIVEWQPKSIRKARIKGMLSLFQTTEDRLSYNFDMYEESIIPEK |
| delARM (Δ2-51) + | LPGGGGFSIKNISLYALYQEHIHAHNIFTHTNTDRPLARYTGAS |
| Buried Cysteines to | LKFYQSKDIDYVVTYSTSLPLRSSMGMYNSMQPSIHLMQQNK |
| Alanines (C63A, | LIVPSKQTQKRRKPYIKKHISPPTQMKSQWYFQHNIANIPLLMI |
| C137A, C269A, | RTTALTLDNYYIGSRQLSTNVTIHTLNTTYIQNRDWGDRNKTY |
| C403A, C460A, | YAQTLGTQRYFLYGTHSTAQNINDIKLQELIPLTNTQDYVQGF |
| C503A, C515A) + | DWTEKDKHNITTYKEFLTKGAGNPFHAEWITAQNPVIHTANSP |
| Exposed Cysteines to | TQIEQIYTASTCTFQNKKLTDLPTPGYIFITPTVSLRYNPYKDLA |
| Serine (C70S) + | ERNKAYFVRSKINAHGWDPEQHQELINSDLPQWLLLFGYPDYI |
| P2 Cysteine for | KRTQNFALVDTNYILVDHAPYTNPEKTPFIPLSTSFIEGRSPYSP |
| conjugation (T365C) | SDTHEPDEEDQNRWYPAYQYQQESINSIALSGPGTPKIPKGITA |
| (SB-7085/pRTx- | EAKVKYSFNFKWGGDLPPMSTITNPTDQPTYVVPNNFNETTSL |
| 2690) | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2560) |
| 5. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTASTCTFQNKKLTDLPTPGYIFITPTVSLRYNPYKDLA |
| conjugation (T365C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2561) |
| 6. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHCTAQNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTASTTTFQNKKLTDLPTPGYIFITPTVSLRYNPYKDLA |
| conjugation (S284C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2562) |
| 6. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNICDIKLQELIPLTNTQDYVQGF |
| C515S) + | DWTEKDKHNITTYKEFLTKGAGNPFHAEWITAQNPVIHTANSP |
| P2 Cysteine for | TQIEQIYTASTTTFQNKKLTDLPTPGYIFITPTVSLRYNPYKDLA |
| conjugation (N290C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2563) |
| 8. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLINTQDYVQGF |
| C515S) + | DWTEKDCHNITTYKEFLTKGAGNPFHAEWITAQNPVIHTANSP |
| P2 Cysteine for | TQIEQIYTASTTTFQNKKLTDLPTPGYIFITPTVSLRYNPYKDLA |
| conjugation (K317C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2564) |
| 9. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLINTQDYVQGF |
| C515S) + | DWTEKDKHNITTYCEFLTKGAGNPFHAEWITAQNPVIHTANSP |
| P2 Cysteine for | TQIEQIYTASTTTFQNKKLTDLPTPGYIFITPTVSLRYNPYKDLA |
| conjugation (K324C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2565) |
| 10. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTCSTTTFQNKKLTDLPTPGYIFITPTVSLRYNPYKDLA |
| conjugation (A362C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2566) |
| 11. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTACTTTFQNKKLTDLPTPGYIFITPTVSLRYNPYKDLA |
| conjugation (S363C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2567) |
| 12. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLINTQDYVQGF |
| C515S) + | DWTEKDKHNITTYKEFLTKGAGNPFHAEWITAQNPVIHTANSP |
| P2 Cysteine for | TQIEQIYTASTTTFQCKKLTDLPTPGYIFITPTVSLRYNPYKDLA |
| conjugation (N369C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2568) |
| 13. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTAQNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTASTTTFQNKCLTDLPTPGYIFITPTVSLRYNPYKDLA |
| conjugation (K371C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2569) |
| 6. Ring10-ORF1 | MIVEWQPKSIRKSRIKGMLSLFQTTEDRLSYNFDMYEESIIPEK |
| delARM (Δ2-51) + | LPGGGGFSIKNISLYALYQEHIHAHNIFTHTNTDRPLARYTGSS |
| All Cysteine to Serine | LKFYQSKDIDYVVTYSTSLPLRSSMGMYNSMQPSIHLMQQNK |
| (C63S, C70S, C137S, | LIVPSKQTQKRRKPYIKKHISPPTQMKSQWYFQHNIANIPLLMI |
| C269S, C403S, | RTTALTLDNYYIGSRQLSTNVTIHTLNTTYIQNRDWGDRNKTY |
| C460S, C503S, | YSQTLGTQRYFLYGTHSTACNINDIKLQELIPLTNTQDYVQGF |
| C515S) + | DWTEKDKHNITTYKEFLTKGAGNPFHAEWITAQNPVIHTANSP |
| P2 Cysteine for | TQIEQIYTASTTTFQNKKLTDLPTPGYIFITPTVSLRYNPYKDLA |
| conjugation (Q287C) | ERNKSYFVRSKINAHGWDPEQHQELINSDLPQWLLLFGYPDYI |
| | KRTQNFALVDTNYILVDHSPYTNPEKTPFIPLSTSFIEGRSPYSP |
| | SDTHEPDEEDQNRWYPSYQYQQESINSISLSGPGTPKIPKGITA |
| | EAKVKYSFNFKWGGDLPPMSTITNPTDQPTYVVPNNFNETTSL |
| | QNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE (SEQ ID NO: 2570) |
| Other P2 residues that | MPWWYRRRSYNPWRRRNWFRRPRKTIYRRYRRRRRWVRRK |
| can be replaced by | PFYKRKIKRLNIVEWQPKSIRKCRIKGMLCLFQTTEDRLSYNFD |
| Cysteines | MYEESIIPEKLPGGGGFSIKNISLYALYQEHIHAHNIFTHTNTDR |
| (Bold/Underlined and | PLARYTGCSLKFYQSKDIDYVVTYSTSLPLRSSMGMYNSMQPS |
| in Red) | IHLMQQNKLIVPSKQTQKRRKPYIKKHISPPTQMKSQWYFQHN |
| Y254, R263, N264, | IANIPLLMIRTTALTLDNYYIGSRQLSTNVTIHTLNTTYIQNRD |
| K265, L272, G273, | WGDRNKTYYCQTLGTQRYFLYGTHSTAQNINDIKLQELIPLT |
| T274, R276, H283, | NTQDYVQGFDWTEKDKHNITTYKEFLTKGAGNPFHAEWITA |
| T285, N288, D291, | QNPVIHTANSPTQIEQIYTASTTTFQNKKLTDLPTPGYIFITPTV |
| Q308, D311, W312, | SLRYNPYKDLAERNKCYFVRSKINAHGWDPEQHQELINSDLP |
| T313, E314, D316, | QWLLLFGYPDYIKRTQNFALVDTNYILVDHCPYTNPEKTPFIPL |
| H318, N319, T321, | STSFIEGRSPYSPSDTHEPDEEDQNRWYPCYQYQQESINSICLS |
| T328, K329, T341, | GPGTPKIPKGITAEAKVKYSFNFKWGGDLPPMSTITNPTDQPT |
| Q343, T354, Q358, | YVVPNNFNETTSLQNPTTRPEHFLYSFDERRGQLTEKATKRLL |
| T361, T364, Q368, | KDWETKETSLLSTEYRFAEPTQTQAPQEDPSSEEEEESNLFERL |
| D374, P376, P378, | LRQRTKQLQLKRRIIQTLKDLQKLE (SEQ ID NQ: 2571) |
| Y380, I381 | |

TABLE B2-4
Ring10 ORF1 molecules with tags (e.g., epitope tags)

| # ID | Sequence |
|---|---|
| 6xHis-flag tag | MGSSHHHHHHGSDYKDDDDKSGSLEVLFQGPSGATMP |
| (“HHHHHH” disclosed as | WWYRRRSYNPWRRRNWFRRPRKTIYRRYRRRRRWVRRKP |
| SEQ ID NO: 2743)on | FYKRKIKRLNIVEWQPKSIRKCRIKGMLCLFQTTEDRLSYNF |
| Ring10 N-terminus | DMYEESIIPEKLPGGGGFSIKNISLYALYQEHIHAHNIFTHTN |
| | TDRPLARYTGCSLKFYQSKDIDYVVTYSTSLPLRSSMGMYN |
| | SMQPSIHLMQQNKLIVPSKQTQKRRKPYIKKHISPPTQMKSQ |
| | WYFQHNIANIPLLMIRTTALTLDNYYIGSRQLSTNVTIHTLN |
| | TTYIQNRDWGDRNKTYYCQTLGTQRYFLYGTHSTAQNINDI |
| | KLQELIPLTNTQDYVQGFDWTEKDKHNITTYKEFLTKGAGN |
| | PFHAEWITAQNPVIHTANSPTQIEQIYTASTTTFQNKKLTDLP |
| | TPGYIFITPTVSLRYNPYKDLAERNKCYFVRSKINAHGWDPE |
| | QHQELINSDLPQWLLLFGYPDYIKRTQNFALVDTNYILVDH |
| | CPYTNPEKTPFIPLSTSFIEGRSPYSPSDTHEPDEEDQNRWYP |
| | CYQYQQESINSICLSGPGTPKIPKGITAEAKVKYSFNFKWGG |
| | DLPPMSTITNPTDQPTYVVPNNFNETTSLQNPTTRPEHFLYS |
| | FDERRGQLTEKATKRLLKDWETKETSLLSTEYRFAEPTQTQ |
| | APQEDPSSEEEEESNLFERLLRQRTKQLQLKRRIIQTLKDLQ |
| | KLE (SEQ ID NO: 2572) |
| 6His-flag tag | MPWWYRRRSYNPWRRRNWFRRPRKTIYRRYRRRRRWVRR |
| (“HHHHHH” disclosed as | KPFYKRKIKRLNIVEWQPKSIRKCRIKGMLCLFQTTEDRLSY |
| SEQ ID NO: 2743) on | NFDMYEESIIPEKLPGGGGFSIKNISLYALYQEHIHAHNIFTH |
| Ring10 C-terminus | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVVPNNFNETTSLQNPTTRPE |
| | HFLYSFDERRGQLTEKATKRLLKDWETKETSLLSTEYRFAE |
| | PTQTQAPQEDPSSEEEEESNLFERLLRQRTKQLQLKRRIIQTL |
| | KDLQKLEGSSHHHHHHGSDYKDDDDKSGSLEVLFQGPS |
| | GAT (SEQ ID NO: 2573) |
| 6His-flag tag | MPWWYRRRSYNPWRRRNWFRRPRKTIYRRYRRRRRWVRR |
| (“HHHHHH” disclosed as | KPFYKRKIKRLNIVEWQPKSIRKCRIKGMLCLFQTTEDRLSY |
| SEQ ID NO: 2743) on | NFDMYEESIIPEKLPGGGGFSIKNISLYALYQEHIHAHNIFTH |
| Ring10 AC-terminus | TNTDRPLARYTGCSLKFYQSKDIDYVVTYSTSLPLRSSMGM |
| (Δ563-672) | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVGSSHHHHHHGSDYKDDD |
| | DKSGSLEVLFQGPSGAT (SEQ ID NO: 2574) |
| SNAP tag attachment on | MPWWYRRRSYNPWRRRNWFRRPRKTIYRRYRRRRRWVRR |
| Ring10 C-terminus | KPFYKRKIKRLNIVEWQPKSIRKCRIKGMLCLFQTTEDRLSY |
| | NFDMYEESIIPEKLPGGGGFSIKNISLYALYQEHIHAHNIFTH |
| | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVVPNNFNETTSLQNPTTRPE |
| | HFLYSFDERRGQLTEKATKRLLKDWETKETSLLSTEYRFAE |
| | PTQTQAPQEDPSSEEEEESNLFERLLRQRTKQLQLKRRIIQTL |
| | KDLQKLEMDKDCEMKRTTLDSPLGKLELSGCEQGLHRII |
| | FLGKGTSAADAVEVPAPAAVLGGPEPLMQATAWLNAYF |
| | HQPEAIEEFPVPALHHPVFQQESFTRQVLWKLLKVVKF |
| | GEVISYSHLAALAGNPAATAAVKTALSGNPVPILIPCHRV |
| | VQGDLDVGGYEGGLAVKEWLLAHEGHRLGKPGLG |
| | (SEQ ID NO: 2575) |
| SNAP tag attach on | MPWWYRRRSYNPWRRRNWFRRPRKTIYRRYRRRRRWVRR |
| Ring 10 (Δ563-672) C- | KPFYKRKIKRLNIVEWQPKSIRKCRIKGMLCLFQTTEDRLSY |
| terminus | NFDMYEESIIPEKLPGGGGFSIKNISLYALYQEHIHAHNIFTH |
| | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVMDKDCEMKRTTLDSPLG |
| | KLELSGCEQGLHRIIFLGKGTSAADAVEVPAPAAVLGGP |
| | EPLMQATAWLNAYFHQPEAIEEFPVPALHHPVFQQESFT |
| | RQVLWKLLKVVKFGEVISYSHLAALAGNPAATAAVKTA |
| | LSGNPVPILIPCHRVVQGDLDVGGYEGGLAVKEWLLAH |
| | EGHRLGKPGLG (SEQ ID NO: 2576) |
| Halo-His tag attach on | MPWWYRRRSYNPWRRRNWFRRPRKTIYRRYRRRRRWVRR |
| Ring 10 (Δ563-672) C- | KPFYKRKIKRLNIVEWQPKSIRKCRIKGMLCLFQTTEDRLSY |
| terminus | NFDMYEESIIPEKLPGGGGFSIKNISLYALYQEHIHAHNIFTH |
| | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVMAEIGTGFPFDPHYVEVL |
| | GERMHYVDVGPRDGTPVLFLHGNPTSSYVWRNIIPHVAP |
| | THRCIAPDLIGMGKSDKPDLGYFFDDHVRFMDAFIEALG |
| | LEEVVLVIHDWGSALGFHWAKRNPERVKGIAFMEFIRPI |
| | PTWDEWPEFARETFQAFRTTDVGRKLIIDQNVFIEGTLP |
| | MGVVRPLTEVEMDHYREPFLNPVDREPLWRFPNELPIA |
| | GEPANIVALVEEYMDWLHQSPVPKLLFWGTPGVLIPPAE |
| | AARLAKSLPNCKAVDIGPGLNLLQEDNPDLIGSEIARWL |
| | STLEIS (SEQ ID NO: 2577) |
| CLIP tag attach on Ring | MPWWYRRRSYNPWRRRNWFRRPRKTIYRRYRRRRRWVRR |
| 10 (Δ563-672) C- | KPFYKRKIKRLNIVEWQPKSIRKCRIKGMLCLFQTTEDRLSY |
| terminus | NEDMYEESIIPEKLPGGGGFSIKNISLYALYQEHIHAHNIFTH |
| | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVMDKDCEMKRTTLDSPLG |
| | KLELSGCEQGLHRIIFLGKGTSAADAVEVPAPAAVLGGP |
| | EPLIQATAWLNAYFHQPEAIEEFPVPALHHPVFQQESFT |
| | RQVLWKLLKVVKFGEVISESHLAALVGNPAATAAVNTA |
| | LDGNPVPILIPCHRVVQGDSDVGPYLGGLAVKEWLLAH |
| | EGHRLGKPGLG (SEQ ID NO: 2578) |
| Ring10 C-terminus | MPWWYRRRSYNPWRRRNWFRRPRKTIYRRYRRRRRWVRR |
| (Δ563-672) + AVI-tag | KPFYKRKIKRLNIVEWQPKSIRKCRIKGMLCLFQTTEDRLSY |
| insertion between 284- | NFDMYEESIIPEKLPGGGGFSIKNISLYALYQEHIHAHNIFTH |
| 285 | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSGLND |
| | IFEAQKIEWHETAQNINDIKLQELIPLTNTQDYVQGFDWTE |
| | KDKHNITTYKEFLTKGAGNPFHAEWITAQNPVIHTANSPTQI |
| | EQIYTASTTTFQNKKLTDLPTPGYIFITPTVSLRYNPYKDLAE |
| | RNKCYFVRSKINAHGWDPEQHQELINSDLPQWLLLFGYPD |
| | YIKRTQNFALVDTNYILVDHCPYTNPEKTPFIPLSTSFIEGRSP |
| | YSPSDTHEPDEEDQNRWYPCYQYQQESINSICLSGPGTPKIP |
| | KGITAEAKVKYSFNFKWGGDLPPMSTITNPTDQPTYV (SEQ |
| | ID NO: 2579) |
| Ring10 delARM (Δ2- | MKRLNIVEWQPKSIRKCRIKGMLCLFQTTEDRLSYNFDMYE |
| 47) C-terminus (Δ563- | ESIIPEKLPGGGGFSIKNISLYALYQEHIHAHNIFTHTNTDRPL |
| 672) + AVI-tag insertion | ARYTGCSLKFYQSKDIDYVVTYSTSLPLRSSMGMYNSMQPS |
| between 284-285 | IHLMQQNKLIVPSKQTQKRRKPYIKKHISPPTQMKSQWYFQ |
| | HNIANIPLLMIRTTALTLDNYYIGSRQLSTNVTIHTLNTTYIQ |
| | NRDWGDRNKTYYCQTLGTQRYFLYGTHSGLNDIFEAQKIE |
| | WHETAQNINDIKLQELIPLTNTQDYVQGFDWTEKDKHNITT |
| | YKEFLTKGAGNPFHAEWITAQNPVIHTANSPTQIEQIYTAST |
| | TTFQNKKLTDLPTPGYIFITPTVSLRYNPYKDLAERNKCYFV |
| | RSKINAHGWDPEQHQELINSDLPQWLLLFGYPDYIKRTQNF |
| | ALVDTNYILVDHCPYTNPEKTPFIPLSTSFIEGRSPYSPSDTHE |
| | PDEEDQNRWYPCYQYQQESINSICLSGPGTPKIPKGITAEAK |
| | VKYSFNFKWGGDLPPMSTITNPTDQPTYV (SEQ ID NO: |
| | 2580) |
| Ring10 C-terminus | MPWWYRRRSYNPWRRRNWFRRPRKTIYRRYRRRRRWVRR |
| (Δ563-672) + AVI-tag | KPFYKRKIKRLNIVEWQPKSIRKCRIKGMLCLFQTTEDRLSY |
| insertion between 328- | NFDMYEESIIPEKLPGGGGFSIKNISLYALYQEHIHAHNIFTH |
| 329 | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLGL |
| | NDIFEAQKIEWHETKGAGNPFHAEWITAQNPVIHTANSPT |
| | QIEQIYTASTTTFQNKKLTDLPTPGYIFITPTVSLRYNPYKDL |
| | AERNKCYFVRSKINAHGWDPEQHQELINSDLPQWLLLFGYP |
| | DYIKRTQNFALVDTNYILVDHCPYTNPEKTPFIPLSTSFIEGR |
| | SPYSPSDTHEPDEEDQNRWYPCYQYQQESINSICLSGPGTPK |
| | IPKGITAEAKVKYSFNFKWGGDLPPMSTITNPTDQPTYV |
| | (SEQ ID NO: 2581) |
| Ring10 delARM (Δ2- | MKRLNIVEWQPKSIRKCRIKGMLCLFQTTEDRLSYNFDMYE |
| 47) C-terminus (Δ563- | ESIIPEKLPGGGGFSIKNISLYALYQEHIHAHNIFTHTNTDRPL |
| 672) + AVI-tag insertion | ARYTGCSLKFYQSKDIDYVVTYSTSLPLRSSMGMYNSMQPS |
| between 328-329 | IHLMQQNKLIVPSKQTQKRRKPYIKKHISPPTQMKSQWYFQ |
| | HNIANIPLLMIRTTALTLDNYYIGSRQLSTNVTIHTLNTTYIQ |
| | NRDWGDRNKTYYCQTLGTQRYFLYGTHSTAQNINDIKLQE |
| | LIPLTNTQDYVQGFDWTEKDKHNITTYKEFLGLNDIFEAQK |
| | IEWHETKGAGNPFHAEWITAQNPVIHTANSPTQIEQIYTAST |
| | TTFQNKKLTDLPTPGYIFITPTVSLRYNPYKDLAERNKCYFV |
| | RSKINAHGWDPEQHQELINSDLPQWLLLFGYPDYIKRTQNF |
| | ALVDTNYILVDHCPYTNPEKTPFIPLSTSFIEGRSPYSPSDTHE |
| | PDEEDQNRWYPCYQYQQESINSICLSGPGTPKIPKGITAEAK |
| | VKYSFNFKWGGDLPPMSTITNPTDQPTYV (SEQ ID NO: |
| | 2582) |
| Ring10 delARM (Δ2- | MKRLNIVEWQPKSIRKCRIKGMLCLFQTTEDRLSYNFDMYE |
| 47) + P2 (Δ256-383) | ESIIPEKLPGGGGFSIKNISLYALYQEHIHAHNIFTHTNTDRPL |
| replaced by | ARYTGCSLKFYQSKDIDYVVTYSTSLPLRSSMGMYNSMQPS |
| miRFP670nano | IHLMQQNKLIVPSKQTQKRRKPYIKKHISPPTQMKSQWYFQ |
| | HNIANIPLLMIRTTALTLDNYYIGSRQLSTNVTIHTLNTTYIA |
| | NLDKMLNTTVTEVRQFLQVDRVCVFQFEEDYSGVVVVE |
| | AVDDRWISILKTQVRDRYFMETRGEEYSHGRYQAIADIY |
| | TANLTECYRDLLTQFQVRAILAVPILQGKKLWGLLVAH |
| | QLAAPRQWQTWEIDFLKQQAVVVGIAIQQSTPTVSLRYN |
| | PYKDLAERNKCYFVRSKINAHGWDPEQHQELINSDLPQWL |
| | LLFGYPDYIKRTQNFALVDTNYILVDHCPYTNPEKTPFIPLST |
| | SFIEGRSPYSPSDTHEPDEEDQNRWYPCYQYQQESINSICLSG |
| | PGTPKIPKGITAEAKVKYSFNFKWGGDLPPMSTITNPTDQPT |
| | YV (SEQ ID NO: 2583) |
| Ring10 delARM (Δ2- | MKRLNIVEWQPKSIRKCRIKGMLCLFQTTEDRLSYNFDMY |
| 47) + ΔC-terminus | EESIIPEKLPGGGGFSIKNISLYALYQEHIHAHNIFTHTNTDR |
| (Δ563-672) + P2 (Δ256- | PLARYTGCSLKFYQSKDIDYVVTYSTSLPLRSSMGMYNSM |
| 383) replaced by | QPSIHLMQQNKLIVPSKQTQKRRKPYIKKHISPPTQMKSQW |
| SpyCatcher03 Protein + | YFQHNIANIPLLMIRTTALTLDNYYIGSRQLSTNVTIHTLNT |
| GGGS linkers | TYIGGGGSVTTLSGLSGEQGPSGDMTTEEDSATHIKFSK |
| | RDEDGRELAGATMELRDSSGKTISTWISDGHVKDFYLY |
| | PGKYTFVETAAPDGYEVATPIEFTVNEDGQVTVDGEAT |
| | EGDAHTGGGGSTPTVSLRYNPYKDLAERNKCYFVRSKIN |
| | AHGWDPEQHQELINSDLPQWLLLFGYPDYIKRTQNFALVD |
| | TNYILVDHCPYTNPEKTPFIPLSTSFIEGRSPYSPSDTHEPDE |
| | EDQNRWYPCYQYQQESINSICLSGPGTPKIPKGITAEAKVK |
| | YSFNFKWGGDLPPMSTITNPTDQPTYV (SEQ ID NO: 2584) |
| GFP linker with the | MPWWYRRRSYNPWRRRNWFRRPRKTIYRRYRRRRRWVRR |
| Ring10 C-terminus | KPFYKRKIKRLNIVEWQPKSIRKCRIKGMLCLFQTTEDRLSY |
| (Δ563-672) | NFDMYEESIIPEKLPGGGGFSIKNISLYALYQEHIHAHNIFTH |
| | TNTDRPLARYTGCSLKFYQSKDIDYVVTYSTSLPLRSSMGM |
| | YNSMQPSIHLMQQNKLIVPSKQTQKRRKPYIKKHISPPTQM |
| | KSQWYFQHNIANIPLLMIRTTALTLDNYYIGSRQLSTNVTIH |
| | TLNTTYIQNRDWGDRNKTYYCQTLGTQRYFLYGTHSTAQN |
| | INDIKLQELIPLTNTQDYVQGFDWTEKDKHNITTYKEFLTKG |
| | AGNPFHAEWITAQNPVIHTANSPTQIEQIYTASTTTFQNKKL |
| | TDLPTPGYIFITPTVSLRYNPYKDLAERNKCYFVRSKINAHG |
| | WDPEQHQELINSDLPQWLLLFGYPDYIKRTQNFALVDTNYI |
| | LVDHCPYTNPEKTPFIPLSTSFIEGRSPYSPSDTHEPDEEDQN |
| | RWYPCYQYQQESINSICLSGPGTPKIPKGITAEAKVKYSFNF |
| | KWGGDLPPMSTITNPTDQPTYVSKGEELFTGVVPILVELD |
| | GDVNGHKFSVSGEGEGDATYGKLTLKFICTTGKLPVPW |
| | PTLVTTLTYGVQCESRYPDHMKQHDFFKSAMPEGYVQE |
| | RTIFFKDDGNYKTRAEVKFEGDTLVNRIELKGIDFKEDG |
| | NILGHKLEYNYNSHNVYIMADKQKNGIKVNFKIRHNIED |
| | GSVQLADHYQQNTPIGDGPVLLPDNHYLSTQSALSKDPN |
| | EKRDHMVLLEFVTAAGITLGMDELYK (SEQ ID NO: 2585) |
| Ring10 delARM (Δ2- | MIVEWQPKSIRKCRIKGMLCLFQTTEDRLSYNFDMYEESIIP |
| 51) + P2 (Δ251-383) | EKLPGGGGFSIKNISLYALYQEHIHAHNIFTHTNTDRPLARY |
| replaced by C-terminus | TGCSLKFYQSKDIDYVVTYSTSLPLRSSMGMYNSMQPSIHL |
| of Malaria CS protein | MQQNKLIVPSKQTQKRRKPYIKKHISPPTQMKSQWYFQHNI |
| | ANIPLLMIRTTALTLDNYYIGSRQLSTNVTIHTLANPNANPN |
| | ANPNANPNANPNANPNANPNANPNANPNANPNANPNANP |
| | NANPNANPNANPNKNNQGNGQGHNMPNDPNRNVDENA |
| | NANSAVKNNNNEEPSDKHIKEYLNKIQNSLSTEWSPCSV |
| | TCGNGIQVRIKPGSANKPKDELDYANDIEKKICKMEKCS |
| | SVFNVVNSSIGLIMVLSFLFLNTPTVSLRYNPYKDLAERNK |
| | CYFVRSKINAHGWDPEQHQELINSDLPQWLLLFGYPDYIKR |
| | TQNFALVDTNYILVDHCPYTNPEKTPFIPLSTSFIEGRSPYSPS |
| | DTHEPDEEDQNRWYPCYQYQQESINSICLSGPGTPKIPKGIT |
| | AEAKVKYSFNFKWGGDLPPMSTITNPTDQPTYVVPNNFNET |
| | TSLQNPTTRPEHFLYSFDERRGQLTEKATKRLLKDWETKET |
| | SLLSTEYRFAEPTQTQAPQEDPSSEEEEESNLFERLLRQRTK |
| | QLQLKRRIIQTLKDLQKLE (SEQ ID NO: 2586) |
| Ring10 delARM (Δ2- | MIVEWQPKSIRKCRIKGMLCLFQTTEDRLSYNFDMYEESIIP |
| 51) + AC-terminus | EKLPGGGGFSIKNISLYALYQEHIHAHNIFTHTNTDRPLARY |
| (Δ563-672) replaced by | TGCSLKFYQSKDIDYVVTYSTSLPLRSSMGMYNSMQPSIHL |
| C-Terminus of Malaria | MQQNKLIVPSKQTQKRRKPYIKKHISPPTQMKSQWYFQHNI |
| CS protein | ANIPLLMIRTTALTLDNYYIGSRQLSTNVTIHTLNTTYIQNRD |
| | WGDRNKTYYCQTLGTQRYFLYGTHSTAQNINDIKLQELIPL |
| | TNTQDYVQGFDWTEKDKHNITTYKEFLTKGAGNPFHAEWI |
| | TAQNPVIHTANSPTQIEQIYTASTTTFQNKKLTDLPTPGYIFIT |
| | PTVSLRYNPYKDLAERNKCYFVRSKINAHGWDPEQHQELIN |
| | SDLPQWLLLFGYPDYIKRTQNFALVDTNYILVDHCPYTNPE |
| | KTPFIPLSTSFIEGRSPYSPSDTHEPDEEDQNRWYPCYQYQQ |
| | ESINSICLSGPGTPKIPKGITAEAKVKYSFNFKWGGDLPPMST |
| | ITNPTDQPTYVNPNANPNANPNANPNANPNANPNANPNAN |
| | PNANPNANPNANPNANPNANPNANPNANPNKNNQGNGQ |
| | GHNMPNDPNRNVDENANANSAVKNNNNEEPSDKHIKEY |
| | LNKIQNSLSTEWSPCSVTCGNGIQVRIKPGSANKPKDELD |
| | YANDIEKKICKMEKCSSVFNVVNSSIGLIMVLSFLFLN |
| | (SEQ ID NO: 2587) |
| Ring10 delARM (Δ2- | MKIKRLNIVEWQPKSIRKCRIKGMLCLFQTTEDRLSYNFDM |
| 45) with Structured | YEESIIPEKLPGGGGFSIKNISLYALYQEHIHAHNIFTHTNTDR |
| Malaria Graft replacing | PLARYTGCSLKFYQSKDIDYVVTYSTSLPLRSSMGMYNSMQ |
| HVR (Δ251-384) | PSIHLMQQNKLIVPSKQTQKRRKPYIKKHISPPTQMKSQWYF |
| | QHNIANIPLLMIRTTALTLDNYYIGSRQLSTNVTIHTLEPSDK |
| | HIKEYLNKIQNSLSTEWSPCSVTCGNGIQVRIKPGSANKP |
| | KDELDYANDIEKKICKMEKCSSPTVSLRYNPYKDLAERNK |
| | CYFVRSKINAHGWDPEQHQELINSDLPQWLLLFGYPDYIKR |
| | TQNFALVDTNYILVDHCPYTNPEKTPFIPLSTSFIEGRSPYSPS |
| | DTHEPDEEDQNRWYPCYQYQQESINSICLSGPGTPKIPKGIT |
| | AEAKVKYSFNFKWGGDLPPMSTITNPTDQPTYVVPNNFNET |
| | TSLQNPTTRPEHFLYSFDERRGQLTEKATKRLLKDWETKET |
| | SLLSTEYRFAEPTQTQAPQEDPSSEEEEESNLFERLLRQRTK |
| | QLQLKRRIIQTLKDLQKLE (SEQ ID NO: 2588) |
| Ring10 delARM (Δ2- | MKIKRLNIVEWQPKSIRKCRIKGMLCLFQTTEDRLSYNFDM |
| 45) with NANPx6 | YEESIIPEKLPGGGGFSIKNISLYALYQEHIHAHNIFTHTNTDR |
| Malaria Graft replacing | PLARYTGCSLKFYQSKDIDYVVTYSTSLPLRSSMGMYNSMQ |
| HVR (Δ251-384) | PSIHLMQQNKLIVPSKQTQKRRKPYIKKHISPPTQMKSQWYF |
| | QHNIANIPLLMIRTTALTLDNYYIGSRQLSTNVTIHTLNANP |
| | NANPNANPNANPNANPNANPPTVSLRYNPYKDLAERNKC |
| | YFVRSKINAHGWDPEQHQELINSDLPQWLLLFGYPDYIKRT |
| | QNFALVDTNYILVDHCPYTNPEKTPFIPLSTSFIEGRSPYSPS |
| | DTHEPDEEDQNRWYPCYQYQQESINSICLSGPGTPKIPKGIT |
| | AEAKVKYSFNFKWGGDLPPMSTITNPTDQPTYVVPNNFNET |
| | TSLQNPTTRPEHFLYSFDERRGQLTEKATKRLLKDWETKET |
| | SLLSTEYRFAEPTQTQAPQEDPSSEEEEESNLFERLLRQRTK |
| | QLQLKRRIIQTLKDLQKLE (SEQ ID NO: 2589) |

TABLE B2-5
Ring2 ORF1 molecules with different
tags on the surface

| | |
|---|---|
| Ring2 delARM (Δ2-45) + | MLKQWQPPYKRTCYIKGQDCLIYYSNL |
| P2 (Δ249-383) | RLGMNSTMYEKSIVPVHWPGGGSFSVS |
| replaced by | MLTLDALYDIHKLCRNWWTSTNQDLPL |
| miRFP670nano + | VRYKGCKITFYQSTFTDYIVRIHTELP |
| delCTerm (611-666) | ANSNKLTYPNTHPLMMMMSKYKHIIPS |
| | RQTRRKKKPYTKIFVKPPPQFENKWYF |
| | ATDLYKIPLLQIHCTACNLQNPFVKPD |
| | KLSNNVTLWSLNTISIQNRNMSVDQGQ |
| | SWPFKILGTQSFYFYFYTGANLPGDTT |
| | QIPVADLLPLTNPRINRPGQSLNEAKI |
| | TDHITFTEYKNKFTNYWGNPFNKHIQE |
| | HLDMILYSLKSPEAIKNEWTTENMKWN |
| | QLNNAGTMALTPFNEPIFTQIQYNPDR |
| | DTGEDTQLYLLSNATGTGWDPPGIPEL |
| | ILEGFPLWLIYWGFADFQKNLKKVTNI |
| | DTNYMLVAKTKFTQKPGTFYLVILNDT |
| | FVEGNSPYEKQPLPEDNIKWYPQVQYQ |
| | LEAQNKLLQTGPFTPNIQGQLSDNISM |
| | FYKFYFKWGGSPPKAINVENPAHQIQY |
| | PIPRNEHETTSLQSPGEAPESILYSFD |
| | YRHGNYTTTALSRISQDWALKDTVSK |
| | (SEQ ID NO: 2590) |

TABLE B2-6
Ring19 ORF1 molecules with different tag attachments

| # ID | Sequence |
|---|---|
| Ring19 delARM (Δ2-46) + | MKITTKEWQPKVIRKTHVKGTYPLFLCTKHRINNNMIQYLDSI |
| P2 (Δ254-370) | APEHYYGGGGFSIMQFSLQALYEEFIKAKNWWTNTNCFLPLV |
| replaced by | RYMGCSFKFYKTEFYDYIVLIERCYPLACTDEMYLSTQPSIMM |
| miRFP670nano | LTRKCIFVPCKQNSKGKKPYKKVRVRPPSQMTTGWHFSQDL |
| | ANMPLVVLKTSVCSFDRYYTDSTAKSTTIGFKTLNTQTFANL |
| | DKMLNTTVTEVRQFLQVDRVCVFQFEEDYSGVVVVEAVD |
| | DRWISILKTQVRDRYFMETRGEEYSHGRYQAIADIYTANLT |
| | ECYRDLLTQFQVRAILAVPILQGKKLWGLLVAHQLAAPRQ |
| | WQTWEIDFLKQQAVVVGIAIQQSPLWLPCRYNPFADKATN |
| | NKIWLVSIHSDHTEWKPIDNPLLQRTDLPLWLLVWGWQDWQ |
| | KKNQQTSQPDINYLTVISSPYISCYPKLDYYVLLDEGFWEGHS |
| | TYIESITDSDKKHWYPKNRFQIETLNLIANTGPGTVKLRENQA |
| | AEGHMVYRFNFKLGGCPAPMEKICDPSKQSKYPIPNNQQQTT |
| | SLQSPENPIQTYLYDFDERRGLLTERATKRIKQDHTSEK |
| | (SEQ ID NO: 2591) |
| Ring_19_Cterm | MPWYPRRRYPRRRYRWLRRWRARRPFRPRYRRRYWVRNYS |
| 599_SL_His | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSALEVLFEGPGSHHHHHHHHH (SEQ ID NO: 2592) |
| Ring_19_Cterm | MPWYPRRRYPRRRYRWLRRWRARRPFRPRYRRRYWVRNYS |
| 599_LL_His | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSAGSSGSAGSSGSALEVLFEGPGSHHHHHHH |
| | HH (SEQ ID NO: 2593) |
| Ring_19_Cterm | MPWYPRRRYPRRRYRWLRRWRARRPFRPRYRRRYWVRNYS |
| 599_SL_GFPHis | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSALEVLFEGPGSMVSKGEELFTGVVPILVEL |
| | DGDVNGHKFSVSGEGEGDATYGKLTLKFICTTGKLPVPW |
| | PTLVTTLTYGVQCESRYPDHMKQHDFFKSAMPEGYVQER |
| | TIFFKDDGNYKTRAEVKFEGDTLVNRIELKGIDFKEDGNIL |
| | GHKLEYNYNSHNVYIMADKQKNGIKVNFKIRHNIEDGSVQ |
| | LADHYQQNTPIGDGPVLLPDNHYLSTQSALSKDPNEKRDH |
| | MVLLEFVTAAGITLGMDELYKHHHHHHHHH (SEQ ID NO: 2594) |
| Ring_19_Cterm | MPWYPRRRYPRRRYRWLRRWRARRPFRPRYRRRYWVRNYS |
| 599_LL_GFPHis | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSAGSSGSAGSSGSALEVLFEGPGSMVSKGEE |
| | LFTGVVPILVELDGDVNGHKFSVSGEGEGDATYGKLTLKF |
| | ICTTGKLPVPWPTLVTTLTYGVQCESRYPDHMKQHDFFKS |
| | AMPEGYVQERTIFFKDDGNYKTRAEVKFEGDTLVNRIELK |
| | GIDFKEDGNILGHKLEYNYNSHNVYIMADKQKNGIKVNFK |
| | IRHNIEDGSVQLADHYQQNTPIGDGPVLLPDNHYLSTQSAL |
| | SKDPNEKRDHMVLLEFVTAAGITLGMDELYKHHHHHHH |
| | HH (SEQ ID NO: 2595) |
| Ring_19_Cterm | MPWYPRRRYPRRRYRWLRRWRARRPFRPRYRRRYWVRNYS |
| 599_SL_HA | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSALEVLFEGPGSYPYDVPDYA (SEQ ID NO: 2596) |
| Ring_19_Cterm | MPWYPRRRYPRRRYRWLRRWRARRPFRPRYRRRYWVRNYS |
| 599_LL_HA | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSAGSSGSAGSSGSALEVLFEGPGSYPYDVPDYA |
| | (SEQ ID NO: 2597) |
| Ring_19_Cterm | MPWYPRRRYPRRRYRWLRRWRARRPFRPRYRRRYWVRNYS |
| 599_SL_FLAG | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSALEVLFEGPGSDYKDDDDK (SEQ ID NO: 2598) |
| Ring_19_Cterm | MPWYPRRRYPRRRYRWLRRWRARRPFRPRYRRRYWVRNYS |
| 599_LL_FLAG | RKRKLFKITTKEWQPKVIRKTHVKGTYPLFLCTKHRINNNMIQ |
| Δ600-655 | YLDSIAPEHYYGGGGFSIMQFSLQALYEEFIKAKNWWTNTNC |
| | FLPLVRYMGCSFKFYKTEFYDYIVLIERCYPLACTDEMYLSTQ |
| | PSIMMLTRKCIFVPCKQNSKGKKPYKKVRVRPPSQMTTGWHF |
| | SQDLANMPLVVLKTSVCSFDRYYTDSTAKSTTIGFKTLNTQTF |
| | RYHDWQEPPTTGYKPQNLLWFYGAENGSPVDPNNTIVSNLIY |
| | LGGTGPYEKGTPIKTNISNYFSEPKLWGNIFHDDYTSGTSPVF |
| | VTNKSPSEIKTAWNTIKDLTVKASGVFTLRTIPLWLPCRYNPF |
| | ADKATNNKIWLVSIHSDHTEWKPIDNPLLQRTDLPLWLLVWG |
| | WQDWQKKNQQTSQPDINYLTVISSPYISCYPKLDYYVLLDEG |
| | FWEGHSTYIESITDSDKKHWYPKNRFQIETLNLIANTGPGTVK |
| | LRENQAAEGHMVYRFNFKLGGCPAPMEKICDPSKQSKYPIPN |
| | NQQQTTSLQSPENPIQTYLYDFDERRGLLTERATKRIKQDHTS |
| | EKTVLPGSSGSAGSSGSAGSSGSALEVLFEGPGSDYKDDDDK |
| | (SEQ ID NO: 2599) |

TABLE B4-1
Replacement of Ring 10 structural
arginine-rich region with BFDV ARM

| | |
|---|---|
| 1. Ring10 | MWGTSNCACAKFQIRRRYARPYRRRHIRRYRRRRRH |
| ARM | FRRRRFTTNRKIKRLNIVEWQPKSIRKCRIKGMLCL |
| replaced with | FQTTEDRLSYNFDMYEESIIPEKLPGGGGFSIKNIS |
| BFDV (1-44) | LYALYQEHIHAHNIFTHTNTDRPLARYTGCSLKFYQ |
| | SKDIDYVVTYSTSLPLRSSMGMYNSMQPSIHLMQQN |
| | KLIVPSKQTQKRRKPYIKKHISPPTQMKSQWYFQHN |
| | IANIPLLMIRTTALTLDNYYIGSRQLSTNVTIHTLN |
| | TTYIQNRDWGDRNKTYYCQTLGTQRYFLYGTHSTAQ |
| | NINDIKLQELIPLTNTQDYVQGFDWTEKDKHNITTY |
| | KEFLTKGAGNPFHAEWITAQNPVIHTANSPTQIEQI |
| | YTASTTTFQNKKLTDLPTPGYIFITPTVSLRYNPYK |
| | DLAERNKCYFVRSKINAHGWDPEQHQELINSDLPQW |
| | LLLFGYPDYIKRTQNFALVDTNYILVDHCPYTNPEK |
| | TPFIPLSTSFIEGRSPYSPSDTHEPDEEDQNRWYPC |
| | YQYQQESINSICLSGPGTPKIPKGITAEAKVKYSFN |
| | FKWGGDLPPMSTITNPTDQPTYVVPNNFNETTSLQN |
| | PTTRPEHFLYSFDERRGQLTEKATKRLLKDWETKET |
| | SLLSTEYRFAEPTQTQAPQEDPSSEEEEESNLFERL |
| | LRQRTKQLQLKRRIIQTLKDLQKLE |
| | (SEQ ID NO: 2600) |

TABLE B4-2
Interchange between different Anellovirus
sequences, e.g., between Ring 2 and 9

| # ID | Sequence |
|---|---|
| (1426) Ring 2 | MGSSHHHHHHGSDYKDDDDKSGSLEVLFQGPSGATMPYYYR |
| Nterminal | RR |
| HISFLAG- | RYNYRRPRWYGRGWIRRPFRRRFRRKRRVRPTYTTIPLKQW |
| 3CProtease- | QPPYKRTCYIKGQDCLIYYSNLRLGMNSTMYEKSIVPVHWP |
| ORF1 | GGGSFSVSMLTLDALYDIHKLCRNWWTSTNQDLPLVRYKGC |
| | KITFYQSTFTDYIVRIHTELPANSNKLTYPNTHPLMMMMSK |
| | YKHIIPSRQTRRKKKPYTKIFVKPPPQFENKWYFATDLYKI |
| | PLLQIHCTACNLQNPFVKPDKLSNNVTLWSLNTISIQNRNM |
| | SVDQGQSWPFKILGTQSFYFYFYTGANLPGDTTQIPVADLL |
| | PLTNPRINRPGQSLNEAKITDHITFTEYKNKFTNYWGNPFN |
| | KHIQEHLDMILYSLKSPEAIKNEWTTENMKWNQLNNAGTMA |
| | LTPFNEPIFTQIQYNPDRDTGEDTQLYLLSNATGTGWDPPG |
| | IPELILEGFPLWLIYWGFADFQKNLKKVTNIDTNYMLVAKT |
| | KFTQKPGTFYLVILNDTFVEGNSPYEKQPLPEDNIKWYPQV |
| | QYQLEAQNKLLQTGPFTPNIQGQLSDNISMFYKFYFKWGGS |
| | PPKAINVENPAHQIQYPIPRNEHETTSLQSPGEAPESILYS |
| | FDYRHGNYTTTALSRISQDWALKDTVSKITEPDRQQLLKQA |
| | LECLQISEETQEKKEKEVQQLISNLRQQQQLYRERIISLLK |
| | DQ (SEQ ID NO: 2601) |
| (1433) Ring 9 | MGSSHHHHHHGSDYKDDDDKSGSLEVLFQGPSGATMPPYWR |
| Nterminal | QK |
| HISFLAG- | YYRRRYRPFSWRTRRIIQRRKRWRYRKPRKTYWRRKLRVRK |
| 3CProtease- | RFYKRKLKKIVLKQFQPKIIRRCTIFGTICLFQGSPERANN |
| ORF1 | NYIQTIYSYVPDKEPGGGGWTLITESLSSLWEDWEHLKNVW |
| | TQSNAGLPLVRYGGVTLYFYQSAYTDYIAQVENCYPMTDTK |
| | YTHADSAPNRMLLKKHVIRVPSRETRKKRKPYKRVRVGPPS |
| | QMQNKWYFQRDICEIPLIMIAATAVDFRYPFCASDCASNNL |
| | TLTCLNPLLFQNQDFDHPSDTQGYFPKPGVYLYSTQRSNKP |
| | SSSDCIYLGNTKDNQEGKSASSLMTLKTQKITDWGNPFWHY |
| | YIDGSKKIFSYFKPPSQLDSSDFEHMTELAEPMFIQVRYNP |
| | ERDTGQGNLIYVTENFRGQHWDPPSSDNLKLDGFPLYDMCW |
| | GFIDWIEKVHETENLLTNYCFCIRSSAFNEKKTVFIPVDHS |
| | FLTGFSPYETPVKSSDQAHWHPQIRFQTKSINDICLTGPGC |
| | ARSPYGNYMQAKMSYKFHVKWGGCPKTYEKPYDPCSQPNWT |
| | IPHNLNETIQIQNPNTCPQTELQEWDWRRDIVTKKAIERIR |
| | QHTEPHETLQISTGSKHNPPVHRQTSPWTDSETDSEEEKDQ |
| | TQEIQIQLNKLRKHQQHLKQQLKQYLKPQNIE (SEQ ID NO: 2602) |
| (1707) Ring 2 | MPPYWRQKYYRRRYRPFSWRTRRIIQRRKRWRYRKPRKTY |
| ORF1 | WRRKLRVRK |
| with ARG arm | RRVRPTYTTIPLKQWQPPYKRTCYIKGQDCLIYYSNLRLGM |
| of Ring 9 (Ring | NSTMYEKSIVPVHWPGGGSFSVSMLTLDALYDIHKLCRNWWTST |
| 291) | NQDLPLVRYKGCKITFYQSTFTDYIVRIHTELPANSNK |
| | LTYPNTHPLMMMMSKYKHIIPSRQTRRKKKPYTKIFVKPPP |
| | QFENKWYFATDLYKIPLLQIHCTACNLQNPFVKPDKLSNNV |
| | TLWSLNTISIQNRNMSVDQGQSWPFKILGTQSFYFYFYTGA |
| | NLPGDTTQIPVADLLPLTNPRINRPGQSLNEAKITDHITFT |
| | EYKNKFTNYWGNPFNKHIQEHLDMILYSLKSPEAIKNEWTT |
| | ENMKWNQLNNAGTMALTPFNEPIFTQIQYNPDRDTGEDTQL |
| | YLLSNATGTGWDPPGIPELILEGFPLWLIYWGFADFQKNLK |
| | KVTNIDTNYMLVAKTKFTQKPGTFYLVILNDTFVEGNSPYE |
| | KQPLPEDNIKWYPQVQYQLEAQNKLLQTGPFTPNIQGQLSD |
| | NISMFYKFYFKWGGSPPKAINVENPAHQIQYPIPRNEHETT |
| | SLQSPGEAPESILYSFDYRHGNYTTTALSRISQDWALKDTV |
| | SKITEPDRQQLLKQALECLQISEETQEKKEKEVQQLISNLR |
| | QQQQLYRERIISLLKDQ (SEQ ID NO: 2603) |
| (1708) Ring 2 | MPPYWRQK |
| ORF1 | YYRRRYRPFSWRTRRIIQRRKRWRYRKPRKTYWRRKLRVRK |
| with ARG arm | RFYKRKLKKIVLKQFQPKIIRRCTIFGTICLFQGSNLRLGM |
| and Beta strands | NSTMYEKSIVPVHWPGGGSFSVSMLTLDALYDIHKLCRNWW |
| 1 + 2 722 | TSTNQDLPLVRYKGCKITFYQSTFTDYIVRIHTELPANSNK |
| epitope of Ring | LTYPNTHPLMMMMSKYKHIIPSRQTRRKKKPYTKIFVKPPP |
| 9 | QFENKWYFATDLYKIPLLQIHCTACNLQNPFVKPDKLSNNV |
| | TLWSLNTISIQNRNMSVDQGQSWPFKILGTQSFYFYFYTGA |
| | NLPGDTTQIPVADLLPLTNPRINRPGQSLNEAKITDHITFT |
| | EYKNKFTNYWGNPFNKHIQEHLDMILYSLKSPEAIKNEWTT |
| | ENMKWNQLNNAGTMALTPFNEPIFTQIQYNPDRDTGEDTQL |
| | YLLSNATGTGWDPPGIPELILEGFPLWLIYWGFADFQKNLK |
| | KVTNIDTNYMLVAKTKFTQKPGTFYLVILNDTFVEGNSPYE |
| | KQPLPEDNIKWYPQVQYQLEAQNKLLQTGPFTPNIQGQLSD |
| | NISMFYKFYFKWGGSPPKAINVENPAHQIQYPIPRNEHETT |
| | SLQSPGEAPESILYSFDYRHGNYTTTALSRISQDWALKDTV |
| | SKITEPDRQQLLKQALECLQISEETQEKKEKEVQQLISNLR |
| | QQQQLYRERIISLLKDQ (SEQ ID NO: 2604) |
| 3. (1721) Ring 9 | MPPYWRQKYYRRRYRPFSWRTRRIIQRRHRWRYRKPRHTYWRR |
| with LYS/HIS | HLRVRHRFYHRHLHHIVLKQFQPKIIRRCTIFGTICLFQGSPERAN |
| mutations in ARG | NNYIQTIYSYVPDKEPGGGGWTLITESLSSLWEDWEHLKNVWTQ |
| arm and first beta | SNAGLPLVRYGGVTLYFYQSAYTDYIAQVENCYPMTDTKYTHA |
| strand | DSAPNRMLLKKHVIRVPSRETRKKRKPYKRVRVGPPSQMQNKW |
| | YFQRDICEIPLIMIAATAVDFRYPFCASDCASNNLTLTCLNPLLFQ |
| | NQDFDHPSDTQGYFPKPGVYLYSTQRSNKPSSSDCIYLGNTKDNQ |
| | EGKSASSLMTLKTQKITDWGNPFWHYYIDGSKKIFSYFKPPSQLD |
| | SSDFEHMTELAEPMFIQVRYNPERDTGQGNLIYVTENFRGQHWD |
| | PPSSDNLKLDGFPLYDMCWGFIDWIEKVHETENLLTNYCFCIRSS |
| | AFNEKKTVFIPVDHSFLTGFSPYETPVKSSDQAHWHPQIRFQTKSI |
| | NDICLTGPGCARSPYGNYMQAKMSYKFHVKWGGCPKTYEKPYD |
| | PCSQPNWTIPHNLNETIQIQNPNTCPQTELQEWDWRRDIVTKKAI |
| | ERIRQHTEPHETLQISTGSKHNPPVHRQTSPWTDSETDSEEEKDQT |
| | QEIQIQLNKLRKHQQHLKQQLKQYLKPQNIE (SEQ ID NO: 2605) |
| 4. (1722) Ring 9 | MWGTSNCACAKFQIRRRYARPYRRRHIRRYRRRRRHFRRRR |
| with ARG arm of | FTTNRRKRFYKRKLKKIVLKQFQPKIIRRCTIFGTICLFQGSPERA |
| BFDV | NNNYIQTIYSYVPDKEPGGGGWTLITESLSSLWEDWEHLKNVWT |
| | QSNAGLPLVRYGGVTLYFYQSAYTDYIAQVENCYPMTDTKYTH |
| | ADSAPNRMLLKKHVIRVPSRETRKKRKPYKRVRVGPPSQMQNK |
| | WYFQRDICEIPLIMIAATAVDFRYPFCASDCASNNLTLTCLNPLLF |
| | QNQDFDHPSDTQGYFPKPGVYLYSTQRSNKPSSSDCIYLGNTKDN |
| | QEGKSASSLMTLKTQKITDWGNPFWHYYIDGSKKIFSYFKPPSQL |
| | DSSDFEHMTELAEPMFIQVRYNPERDTGQGNLIYVTENFRGQHW |
| | DPPSSDNLKLDGFPLYDMCWGFIDWIEKVHETENLLTNYCFCIRS |
| | SAFNEKKTVFIPVDHSFLTGFSPYETPVKSSDQAHWHPQIRFQTKS |
| | INDICLTGPGCARSPYGNYMQAKMSYKFHVKWGGCPKTYEKPY |
| | DPCSQPNWTIPHNLNETIQIQNPNTCPQTELQEWDWRRDIVTKKA |
| | IERIRQHTEPHETLQISTGSKHNPPVHRQTSPWTDSETDSEEEKDQ |
| | TQEIQIQLNKLRKHQQHLKQQLKQYLKPQNIE (SEQ ID NO: 2606) |
| 5. (1723) Ring 9 | MPPYWRQKYYRRRYRPFSWRTRRIIQRRKRWRYRKPRKTYWRR |
| with beta strands F | KLRVRKRFYKRKLKKIVLKQFQPKIIRRCTIFGTICLFQGSPERAN |
| and G of BFDV | NNYIQTIYSYVPDKEPGGGGWTLITESLSSLWEDWEHLKNVWTQ |
| capsid protein | SNAGLPLVRYGGVTLYFYQSAYTDYIAQVENCYPMTDTKYTHA |
| | DSAPNRMLLKKHAKKWFSRETRKKRKPGFKRLLGPPSQMQNK |
| | WYFQRDICEIPLIMIAATAVDFRYPFCASDCASNNLTLTCLNPLLF |
| | QNQDFDHPSDTQGYFPKPGVYLYSTQRSNKPSSSDCIYLGNTKDN |
| | QEGKSASSLMTLKTQKITDWGNPFWHYYIDGSKKIFSYFKPPSQL |
| | DSSDFEHMTELAEPMFIQVRYNPERDTGQGNLIYVTENFRGQHW |
| | DPPSSDNLKLDGFPLYDMCWGFIDWIEKVHETENLLTNYCFCIRS |
| | SAFNEKKTVFIPVDHSFLTGFSPYETPVKSSDQAHWHPQIRFQTKS |
| | INDICLTGPGCARSPYGNYMQAKMSYKFHVKWGGCPKTYEKPY |
| | DPCSQPNWTIPHNLNETIQIQNPNTCPQTELQEWDWRRDIVTKKA |
| | IERIRQHTEPHETLQISTGSKHNPPVHRQTSPWTDSETDSEEEKDQ |
| | TQEIQIQLNKLRKHQQHLKQQLKQYLKPQNIE (SEQ ID NO: 2607) |
| 6. (1724) Ring 2 | MPYYYRRRRYNYRRPRWYGRGWIRRPFRRRFRRKRRVRPTYTTI |
| with beta C of | PLKQWQPPYKRRCTIFGTICLFQGSNLRLGMNSTMYEKSIVPVH |
| Ring 9 | WPGGGSFSVSMLTLDALYDIHKLCRNWWTSTNQDLPLVRYKGC |
| | KITFYQSTFTDYIVRIHTELPANSNKLTYPNTHPLMMMMSKYKHII |
| | PSRQTRRKKKPYTKIFVKPPPQFENKWYFATDLYKIPLLQIHCTAC |
| | NLQNPFVKPDKLSNNVTLWSLNTISIQNRNMSVDQGQSWPFKILG |
| | TQSFYFYFYTGANLPGDTTQIPVADLLPLTNPRINRPGQSLNEAKI |
| | TDHITFTEYKNKFTNYWGNPFNKHIQEHLDMILYSLKSPEAIKNE |
| | WTTENMKWNQLNNAGTMALTPFNEPIFTQIQYNPDRDTGEDTQL |
| | YLLSNATGTGWDPPGIPELILEGFPLWLIYWGFADFQKNLKKVTN |
| | IDTNYMLVAKTKFTQKPGTFYLVILNDTFVEGNSPYEKQPLPEDN |
| | IKWYPQVQYQLEAQNKLLQTGPFTPNIQGQLSDNISMFYKFYFK |
| | WGGSPPKAINVENPAHQIQYPIPRNEHETTSLQSPGEAPESILYSFD |
| | YRHGNYTTTALSRISQDWALKDTVSKITEPDRQQLLKQALECLQI |
| | SEETQEKKEKEVQQLISNLRQQQQLYRERIISLLKDQ |
| | (SEQ ID NO: 2608) |
| 7. | MPYYYRRRRYNYRRPRWYGRGWIRRPFRRRFRRKRRVRPTYTTI |
| (1725) Ring 2 with | PLKQWQPPYKRTCYIKGQDCLIYYSPERANNNYIQTIYSYVPDK |
| linker 1 of Ring 9 | EPGGGGWTLITESLSSLWEDWEHLKNVWTQSNAGLPLVRYK |
| | GCKITFYQSTFTDYIVRIHTELPANSNKLTYPNTHPLMMMMSKYK |
| | HIIPSRQTRRKKKPYTKIFVKPPPQFENKWYFATDLYKIPLLQIHCT |
| | ACNLQNPFVKPDKLSNNVTLWSLNTISIQNRNMSVDQGQSWPFK |
| | ILGTQSFYFYFYTGANLPGDTTQIPVADLLPLTNPRINRPGQSLNE |
| | AKITDHITFTEYKNKFTNYWGNPFNKHIQEHLDMILYSLKSPEAIK |
| | NEWTTENMKWNQLNNAGTMALTPFNEPIFTQIQYNPDRDTGEDT |
| | QLYLLSNATGTGWDPPGIPELILEGFPLWLIYWGFADFQKNLKKV |
| | TNIDTNYMLVAKTKFTQKPGTFYLVILNDTFVEGNSPYEKQPLPE |
| | DNIKWYPQVQYQLEAQNKLLQTGPFTPNIQGQLSDNISMFYKFYF |
| | KWGGSPPKAINVENPAHQIQYPIPRNEHETTSLQSPGEAPESILYSF |
| | DYRHGNYTTTALSRISQDWALKDTVSKITEPDRQQLLKQALECL |
| | QISEETQEKKEKEVQQLISNLRQQQQLYRERIISLLKDQ |
| | (SEQ ID NO: 2609) |
| 8. (1726) Ring 2 | MPYYYRRRRYNYRRPRWYGRGWIRRPFRRRFRRKRRVRPTYTTI |
| with beta strand D | PLKQWQPPYKRTCYIKGQDCLIYYSNLRLGMNSTMYEKSIVPVH |
| of Ring 9 | WPGGGSFSVSMLTLDALYDIHKLCRNWWTSTNQDLPLVRYGG |
| | VTLYFYQSTFTDYIVRIHTELPANSNKLTYPNTHPLMMMMSKYK |
| | HIIPSRQTRRKKKPYTKIFVKPPPQFENKWYFATDLYKIPLLQIHCT |
| | ACNLQNPFVKPDKLSNNVTLWSLNTISIQNRNMSVDQGQSWPFK |
| | ILGTQSFYFYFYTGANLPGDTTQIPVADLLPLTNPRINRPGQSLNE |
| | AKITDHITFTEYKNKFTNYWGNPFNKHIQEHLDMILYSLKSPEAIK |
| | NEWTTENMKWNQLNNAGTMALTPFNEPIFTQIQYNPDRDTGEDT |
| | QLYLLSNATGTGWDPPGIPELILEGFPLWLIYWGFADFQKNLKKV |
| | TNIDTNYMLVAKTKFTQKPGTFYLVILNDTFVEGNSPYEKQPLPE |
| | DNIKWYPQVQYQLEAQNKLLQTGPFTPNIQGQLSDNISMFYKFYF |
| | KWGGSPPKAINVENPAHQIQYPIPRNEHETTSLQSPGEAPESILYSF |
| | DYRHGNYTTTALSRISQDWALKDTVSKITEPDRQQLLKQALECL |
| | QISEETQEKKEKEVQQLISNLRQQQQLYRERIISLLKDQ |
| | (SEQ ID NO: 2610) |
| 9. (1727) Ring 2 | MPYYYRRRRYNYRRPRWYGRGWIRRPFRRRFRRKRRVRPTYTTI |
| with linker 2 of | PLKQWQPPYKRTCYIKGQDCLIYYSNLRLGMNSTMYEKSIVPVH |
| Ring 9 | WPGGGSFSVSMLTLDALYDIHKLCRNWWTSTNQDLPLVRYKGC |
| | KITFYQSTFTDYIVRIHNCYPMTDTKYTHADSAPNRMLLKKHK |
| | HIIPSRQTRRKKKPYTKIFVKPPPQFENKWYFATDLYKIPLLQIHCT |
| | ACNLQNPFVKPDKLSNNVTLWSLNTISIQNRNMSVDQGQSWPFK |
| | ILGTQSFYFYFYTGANLPGDTTQIPVADLLPLTNPRINRPGQSLNE |
| | AKITDHITFTEYKNKFTNYWGNPFNKHIQEHLDMILYSLKSPEAIK |
| | NEWTTENMKWNQLNNAGTMALTPFNEPIFTQIQYNPDRDTGEDT |
| | QLYLLSNATGTGWDPPGIPELILEGFPLWLIYWGFADFQKNLKKV |
| | TNIDTNYMLVAKTKFTQKPGTFYLVILNDTFVEGNSPYEKQPLPE |
| | DNIKWYPQVQYQLEAQNKLLQTGPFTPNIQGQLSDNISMFYKFYF |
| | KWGGSPPKAINVENPAHQIQYPIPRNEHETTSLQSPGEAPESILYSF |
| | DYRHGNYTTTALSRISQDWALKDTVSKITEPDRQQLLKQALECL |
| | QISEETQEKKEKEVQQLISNLRQQQQLYRERIISLLKDQ |
| | (SEQ ID NO: 2611) |
| 10. (1728) Ring 2 | MPYYYRRRRYNYRRPRWYGRGWIRRPFRRRFRRKRRVRPTYTTI |
| with beta strand G | PLKQWQPPYKRTCYIKGQDCLIYYSNLRLGMNSTMYEKSIVPVH |
| DNA binding of | WPGGGSFSVSMLTLDALYDIHKLCRNWWTSTNQDLPLVRYKGC |
| Ring 9 | KITFYQSTFTDYIVRIHTELPANSNKLTYPNTHPLMMMMSKYKHII |
| | PSRQTRRKKKPYKRVRVKPPPQFENKWYFATDLYKIPLLQIHCT |
| | ACNLQNPFVKPDKLSNNVTLWSLNTISIQNRNMSVDQGQSWPFK |
| | ILGTQSFYFYFYTGANLPGDTTQIPVADLLPLTNPRINRPGQSLNE |
| | AKITDHITFTEYKNKFTNYWGNPFNKHIQEHLDMILYSLKSPEAIK |
| | NEWTTENMKWNQLNNAGTMALTPFNEPIFTQIQYNPDRDTGEDT |
| | QLYLLSNATGTGWDPPGIPELILEGFPLWLIYWGFADFQKNLKKV |
| | TNIDTNYMLVAKTKFTQKPGTFYLVILNDTFVEGNSPYEKQPLPE |
| | DNIKWYPQVQYQLEAQNKLLQTGPFTPNIQGQLSDNISMFYKFYF |
| | KWGGSPPKAINVENPAHQIQYPIPRNEHETTSLQSPGEAPESILYSF |
| | DYRHGNYTTTALSRISQDWALKDTVSKITEPDRQQLLKQALECL |
| | QISEETQEKKEKEVQQLISNLRQQQQLYRERIISLLKDQ |
| | (SEQ ID NO: 2773) |
| 11. (1729) Ring 2 | MPYYYRRRRYNYRRPRWYGRGWIRRPFRRRFRRKRRVRPTYTTI |
| with beta strand F | PLKQWQPPYKRTCYIKGQDCLIYYSNLRLGMNSTMYEKSIVPVH |
| interprotomer | WPGGGSFSVSMLTLDALYDIHKLCRNWWTSTNQDLPLVRYKGC |
| contact of Ring 9 | KITFYQSTFTDYIVRIHTELPANSNKLTYPNTHPLMMMMSKHVIR |
| | VPSRQTRRKKKPYTKIFVKPPPQFENKWYFATDLYKIPLLQIHCT |
| | ACNLQNPFVKPDKLSNNVTLWSLNTISIQNRNMSVDQGQSWPFK |
| | ILGTQSFYFYFYTGANLPGDTTQIPVADLLPLTNPRINRPGQSLNE |
| | AKITDHITFTEYKNKFTNYWGNPFNKHIQEHLDMILYSLKSPEAIK |
| | NEWTTENMKWNQLNNAGTMALTPFNEPIFTQIQYNPDRDTGEDT |
| | QLYLLSNATGTGWDPPGIPELILEGFPLWLIYWGFADFQKNLKKV |
| | TNIDTNYMLVAKTKFTQKPGTFYLVILNDTFVEGNSPYEKQPLPE |
| | DNIKWYPQVQYQLEAQNKLLQTGPFTPNIQGQLSDNISMFYKFYF |
| | KWGGSPPKAINVENPAHQIQYPIPRNEHETTSLQSPGEAPESILYSF |
| | DYRHGNYTTTALSRISQDWALKDTVSKITEPDRQQLLKQALECL |
| | QISEETQEKKEKEVQQLISNLRQQQQLYRERIISLLKDQ |
| | (SEQ ID NO: 2612) |
| 12. (1730) Ring 2 | MPYYYRRRRYNYRRPRWYGRGWIRRPFRRRFRRKRRVRPTYTTI |
| ORF1 with strand | PLKQWQPPYKRTCYIKGQDCLIYYSNLRLGMNSTMYEKSIVPVH |
| H and I with C- | WPGGGSFSVSMLTLDALYDIHKLCRNWWTSTNQDLPLVRYKGC |
| terminal fragment | KITFYQSTFTDYIVRIHTELPANSNKLTYPNTHPLMMMMSKYKHII |
| of Ring 9 | PSRQTRRKKKPYTKIFVKPPPQFENKWYFATDLYKIPLIMIAATA |
| | VDFRYPFCASDCASNNLTLTCLNPLLFQNQDFDHPSDTQGYFP |
| | KPGVYLYSTQRSNKPSSSDCIYLGNTKDNQEGKSASSLMTLKT |
| | QKITDWGNPFWHYYIDGSKKIFSYFKPPSQLDSSDFEHMTELA |
| | EPMFIQVRYNPERDTGQGNLIYVTENFRGQHWDPPSSDNLKL |
| | DGFPLYDMCWGFIDWIEKVHETENLLTNYCFCIRSSAFNEKK |
| | TVFIPVDHSFLTGFSPYETPVKSSDQAHWHPQIRFQTKSINDIC |
| | LTGPGCARSPYGNYMQAKMSYKFHVKWGGCPKTYEKPYDP |
| | CSQPNWTIPHNLNETIQIQNPNTCPQTELQEWDWRRDIVTKK |
| | AIERIRQHTEPHETLQISTGSKHNPPVHRQTSPWTDSETDSEE |
| | EKDQTQEIQIQLNKLRKHQQHLKQQLKQYLKPQNIE |
| | (SEQ ID NO: 2613) |

TABLE B4-3
Replacement of hepatitis E virus domains with Anellovirus P2 domain. The engineering
of virus capsid protein could expand tropism and alleviate severe immune response.
Hepatitis E virus (HEV) is an non-enveloped RNA virus which contains
three structural domains: S (shell), M (middle) and P (protruding)
domains. Here we propose to swap HEV domains with Anellovirus P2 domain to
pursue another novel VLP for encapsulation of mRNA payload.

| | |
|---|---|
| HEV P domain (462-608) | MAVAPAPDTAPVPDVDSRGAILRRQYNLSTSPLTSTIATGTNL |
| replaced with | VLYAAPLSSLLPLQDGTNTHIMATEASNYAQYRVVRATIRYR |
| Anellovirus P2 (250- | PLVPSAVGGYAISISFWPQTTTTPTSVDMNSITSTDVRILVQPGI |
| 387) | ASELVIPSERLHYRNQGWRSVETSGVAEEEATSGLVMLCIHGS |
| | PVNSYTNTPYTGALGLLDFALELEFRNLTPGNTNTRVSRYSSS |
| | ARHKLRRGPDGTAELTTTAATRFMKDLHFTGTNDVGEVGRG |
| | IALTLFNLADTLLGGLPTELISSAGGQLFYSRPVVSANGEPTVK |
| | LYTSVENAQQDKGIAIPHDIDLGESRVVIQDYDNQHEQDRPTP |
| | SPAPSRPLNTTYIQNRDWGDRNKTYYCQTLGTQRYFLYGT |
| | HSTAQNINDIKLQELIPLTNTQDYVQGFDWTEKDKHNITT |
| | YKEFLTKGAGNPFHAEWITAQNPVIHTANSPTQIEQIYTAS |
| | TTTFQNKKLTDLPTPGYIFITPTVHHHHH (SEQ ID NO: 2614) |

Identification of ORF1 protein sequences

In some embodiments, an ORF1 protein sequence, or a nucleic acid sequence encoding an ORF1 protein, can be identified from the genome of an Anelloviridae family virus, e.g., an Anellovirus (e.g., a putative Anelloviridae family virus genome identified, for example, by nucleic acid sequencing techniques, e.g., deep sequencing techniques). In some embodiments, an ORF1 protein sequence is identified by one or more (e.g., 1, 2, or all 3) of the following selection criteria:

(i) Length Selection: Protein sequences (e.g., putative ORF1 sequences passing the criteria described in (ii) or (iii) below) may be size-selected for those greater than about 600 amino acid residues to identify putative ORF1 proteins. In some embodiments, an ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.

(ii) Presence of ORF1 motif Protein sequences (e.g., putative ORF1 sequences passing the criteria described in (i) above or (iii) below) may be filtered to identify those that contain the conserved ORF1 motif in the structural N22 domain described above. In some embodiments, a putative Anellovirus ORF1 sequence comprises the sequence YNPXXDXGXXN (SEQ ID NO: 2615). In some embodiments, a putative Anellovirus ORF1 sequence comprises the sequence Y[NCS]PXXDX[GASKR]XX[NTSVAK].

(iii) Presence of arginine-rich region: Protein sequences (e.g., putative ORF1 sequences passing the criteria described in (i) and/or (ii) above) may be filtered for those that include a structural arginine-rich region (e.g., as described herein). In some embodiments, a putative ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the structural arginine-rich region is positioned at least about 30, 40, 50, 60, 70, or 80 amino acids downstream of the start codon of the putative ORF1 protein. In some embodiments, the structural arginine-rich region is positioned at least about 50 amino acids downstream of the start codon of the putative ORF1 protein.

In some embodiments, an ORF1 protein is identified in an Anellovirus genome sequence as described in Example 36 of PCT Publication No. WO2020/123816 (incorporated herein by reference in its entirety).

### Structural Arginine-Rich Region Deletions and Truncations

In some embodiments, an ORF1 molecule as described herein comprises a deletion or truncation of a structural arginine-rich region. In some embodiments, the entire structural arginine-rich region is deleted. In embodiments, the ORF1 molecule does not comprise an Anellovirus ORF1 structural arginine-rich region, or an amino acid sequence having at least 70%, 75%, 80%, 8%, 90%, 95%, 96%, 97%, 98%, or 99% sequence identity thereto. In embodiments, the ORF1 molecule does not comprise the amino acid sequence of the full-length structural arginine-rich region of an Ring2, Ring9, Ring10, Ring 18, or Ring19 Anellovirus ORF1 protein (e.g., as described herein). In some embodiments, the ORF1 molecule as described herein does not comprise a sequence of at least 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous amino acids consisting of at least 40%, 41%, 42%, 43%, 44%, 45%, 46%, 47%, 48%, 49%, 50%, 51%, 52%, 53%, 54%, 55%, 56%, 57%, 58%, 59%, or 60% basic residues (e.g., arginine and/or lysine residues).

In some embodiments, a portion of the structural arginine-rich region (e.g., a N-terminal portion of the structural arginine-rich region) is deleted. In some embodiments, the ORF1 molecule comprises a portion of a structural arginine-rich region of an Anellovirus ORF1 molecule, which comprises a deletion of about 1-5, 5-10, 10-15, 15-20, 20-25, 25-30, 30-35, or 35-40 amino acids, e.g., at the N-terminal end, of the structural arginine-rich region, relative to a corresponding wild-type structural arginine-rich region of the Anellovirus ORF1 molecule, or an amino acid sequence having at least 70%, 75%, 80%, 8%, 90%, 95%, 96%, 97%, 98%, or 99% sequence identity thereto. In some embodiments, the ORF1 molecule does not comprise the 1, 2, 3, 4, 5, 6, 7, 8, 9 or 10 most N-terminal amino acid residues of the structural jelly-roll region of the Anellovirus ORF1 molecule other than the N-terminal methionine residue. It is understood that the ORF1 molecule generally comprises at its N-terminus a methionine residue, e.g., a methionine residues corresponding to the N-terminal methionine residue of an Anellovirus structural arginine-rich region

In embodiments, the ORF1 molecule comprises a portion of a structural arginine-rich region (e.g., an C-terminal portion of the structural arginine-rich region) consisting of between 1-5, 5-10, 10-15, 15-20, 20-25, 25-30, 30-35, or 35-40 contiguous amino acids of a structural arginine-rich region sequence as described herein, or an amino acid sequence having at least 70%, 75%, 80%, 8%, 90%, 95%, 96%, 97%, 98%, or 99% sequence identity thereto. In embodiments, the ORF1 molecule comprises a portion of a structural arginine-rich region of an Anellovirus ORF1 molecule, wherein the portion consists of the N-terminal most 30-40, 40-50, 50-60, 60-70 (e.g., about 69), 70-80, 80-90, 90-100 (e.g., about 93), or 100-110 amino acids of a corresponding wild-type structural arginine-rich region of an Anellovirus ORF1 molecule, or a sequence having at least 80%, 85%, 90%, 95%, 96%, 97%, 98%, or 99% identity thereto.

In some embodiments, an ORF1 molecule having a deletion or truncation of the structural arginine-rich region further comprises a deletion or truncation of at least a portion of a structural jelly-roll domain as described herein. In some embodiments, an ORF1 molecule having a deletion or truncation of the structural arginine-rich region further comprises a deletion or truncation of at least a portion of a jelly-roll B—H strands subdomain as described herein.

In some embodiments, an ORF1 molecule having a deletion or truncation of the structural arginine-rich region further comprises a deletion or truncation of at least a portion of a structural C-terminal domain (e.g., as described herein).

In some embodiments, an ORF1 molecule comprises an amino acid sequence comprising substitutions of at least 50%, 60%, 70%, 80%, or 90% of basic amino acids (e.g., arginines and/or lysines) relative to the structural arginine-rich region of a wild-type Anellovirus ORF1 molecule.

### Structural C-Terminal Domain Deletions and Truncations

In some embodiments, an ORF1 molecule as described herein comprises a deletion or truncation of a structural C-terminal domain (CTD). In some embodiments, the entire structural CTD is deleted. In embodiments, the ORF1 molecule does not comprise an Anellovirus ORF1 structural CTD, or an amino acid sequence having at least 70%, 75%, 80%, 85%, 90%, 95%, 96%, 97%, 98%, or 99% sequence identity thereto. In embodiments, the ORF1 molecule does not comprise the amino acid sequence of the full-length structural C-terminal domain of an Ring2, Ring9, Ring10, Ring 18, or Ring19 Anellovirus ORF1 protein (e.g., as described herein).

In some embodiments, a portion of the structural CTD (e.g., a C-terminal portion of the structural CTD) is deleted. In some embodiments, the ORF1 molecule comprises a portion of a structural C-terminal domain (CTD) of an Anellovirus ORF1 molecule, which comprises a deletion of about 20-30, 30-40 (e.g., about 37), 40-50 (e.g., about 55), 50-60, 60-70, 70-80, 80-90, 90-100, 100-110, 110-120, 120-130 (e.g., about 129), 130-140 (e.g., about 131), 140-150 (e.g., about 148), or 150-160 (e.g., about 155) amino acids at the C-terminal end of the structural CTD, relative to a corresponding wild-type structural CTD of the Anellovirus ORF1 molecule, or an amino acid sequence having at least 70%, 75%, 80%, 8%, 90%, 95%, 96%, 97%, 98%, or 99% sequence identity thereto.

In embodiments, the ORF1 molecule comprises a portion of a structural CTD (e.g., an N-terminal portion of the structural CTD) consisting of between 1-5, 5-10, 10-15, 15-20, 20-25, 25-30, 30-40, 40-50, 50-60, 60-70, 70-80, 80-90, 90-100, 100-110, 110-120, 120-130, 130-140, or 140-150 contiguous amino acids of a structural CTD sequence as described herein, or an amino acid sequence having at least 70%, 75%, 80%, 8%, 90%, 95%, 96%, 97%, 98%, or 99% sequence identity thereto. In embodiments, the ORF1 molecule comprises a portion of a structural CTD of an Anellovirus ORF1 molecule, wherein the portion consists of the N-terminal most 60-70 (e.g., about 69), 70-80, 80-90, 90-100 (e.g., about 93), or 100-110 amino acids of a corresponding wild-type structural CTD of an Anellovirus ORF1 molecule, or a sequence having at least 80%, 85%, 90%, 95%, 96%, 97%, 98%, or 99% identity thereto.

In some embodiments, an ORF1 molecule having a deletion or truncation of the structural CTD further comprises a deletion or truncation of at least a portion of a structural N22 domain as described herein. In some embodiments, an ORF1 molecule having a deletion or truncation of the structural CTD further comprises a deletion or truncation of at least a portion of a jelly-roll I strand subdomain and/or a P1-2 domain as described herein.

In some embodiments, an ORF1 molecule having a deletion or truncation of the structural CTD further comprises a deletion or truncation of at least a portion of a structural arginine-rich region (e.g., as described herein).

### Chimeric ORF1 Molecules

In some embodiments, an ORF1 molecule described herein comprises a heterologous amino acid sequence (e.g., an amino acid sequence from a protein other than an Anellovirus protein). In some embodiments, the ORF1 molecule comprises one or more deletions or truncations relative to a corresponding Anellovirus ORF1 protein (e.g., a deleteion or truncation of a structural arginine-rich region and/or a deleteion or truncation of a structural CTD, e.g., as described herein). In some embodiments, the heterologous amino acid sequence is inserted at or within 1, 2, 3, 4, 5, 6, 7, 8, 9, 10, 15, 20, 25, 30, 35, 40, 45, or 50 amino acid residues of the position of one of the deletions or truncations relative to the corresponding Anellovirus ORF1 protein. For example, the heterologous amino acid sequence can, in some instances, be inserted at the N-terminus of the ORF1 molecule (e.g., an ORF1 molecule comprising a deletion or truncation of the structural arginine-rich region, e.g., as described herein). In another example, the heterologous amino acid sequence can, in some instances, be inserted at the C-terminus of the ORF1 molecule (e.g., an ORF1 molecule comprising a deletion or truncation of the structural CTD, e.g., as described herein).

In some embodiments, the heterologous amino acid sequence is attached to the remainder of the ORF1 molecule by a linker. In certain embodiments, the linker comprises one or more (e.g., at least 1, 2, 3, 4, or 5) copies of the amino acid sequence GGGGS (SEQ ID NO: 2744). In certain embodiments, the linker comprises the amino acid sequence TYTTIP (SEQ ID NO: 2745).

### N-Terminal Insertions (e.g., Structural Arginine-Rich Region Swaps)

In some embodiments, an ORF1 molecule as described herein comprises a heterologous amino acid sequence at or within 1, 2, 3, 4, 5, 6, 7, 8, 9, 10, 15, 20, 25, 30, 35, 40, or 50 amino acids of its N-terminus. In some embodiments, the structural arginine-rich region of the ORF1 molecule, or a portion thereof (e.g., consisting of at least about 5, 10, 15, 20, 25, 30, 35, or 40 contiguous amino acids, e.g., of the C-terminal portion of the structural arginine-rich region) is replaced by the heterologous amino acid sequence. In some embodiments, the heterologous amino acid sequence is from another virus (e.g., a non-Anellovirus). In certain embodiments, the heterologous amino acid sequence comprises an arginine-rich motif or arginine-rich region from the other virus, or an amino acid sequence having at least 75%, 80%, 85%, 90%, 95%, 96%, 97%, 98%, or 99% identity thereto.

In some embodiments, an N-terminal domain or portion thereof of a heterologous protein (e.g., a capsid protein from a virus other than an Anellovirus, e.g., as described herein), or a sequence having at least 80%, 85%, 90%, 95%, 96%, 97%, 98%, or 99% identity thereto, is inserted into the sequence of the ORF1 molecule (e.g., at the N-terminus and/or at the position of a deletion or truncation of the structural arginine-rich region). In some embodiments, an arginine-rich region or arginine-rich motif, or a portion thereof, of a heterologous protein, or a sequence having at least 80%, 85%, 90%, 95%, 96%, 97%, 98%, or 99% identity thereto, is inserted into the sequence of the ORF1 molecule (e.g., at the N-terminus and/or at the position of a deletion or truncation of the structural arginine-rich region). In some embodiments, the heterologous protein is a capsid protein (e.g., an ORF1 protein) from a different Anellovirus. In certain embodiments the ORF1 molecule comprises a structural arginine-rich region, or a portion thereof, from a different Anellovirus (e.g., replacing one or more of the corresponding residues of the structural arginine-rich region of the ORF1 molecule itself).

In other embodiments, the heterologous protein is a capsid protein from a virus other than an Anellovirus, e.g., as described herein. In certain embodiments, the virus is a beak and feather disease virus (BFDV). In certain embodiments, the ORF1 molecule comprises (e.g., at the N-terminus) the amino acid sequence MWGTSNCACAKFQIRRRYARPYRRRHIRRYRRRRRHFRRRRFTTNR (SEQ ID NO: 2617), or an amino acid sequence having at least 75%, 80%, 85%, 90%, 95%, 96%, 97%, 98%, or 99% identity thereto.

### P1/P2 Domain Swaps

An ORF1 molecule as described herein may, in some instances, comprise a P1 domain, or a functional variant thereof, from the capsid protein of a different virus (e.g., a different Anellovirus or a non-Anellovirus), or an amino acid sequence having at least 75%, 80%, 85%, 90%, 95%, 96%, 97%, 98%, or 99% identity thereto. In certain embodiments, the P1 domain is noncontiguous (e.g., comprising a P1-1 domain and a P1-2 domain, e.g., as described herein). In certain embodiments, the P1 domain or functional fragment thereof from the other virus replaces one or more of the corresponding residues of the P1 domain of the ORF1 molecule. In some instances, an ORF1 molecule as described herein may comprise a P2 domain, or a functional fragment thereof, from the capsid protein of a different virus (e.g., a different Anellovirus or a non-Anellovirus), or an amino acid sequence having at least 75%, 80%, 85%, 90%, 95%, 96%, 97%, 98%, or 99% identity thereto. In certain embodiments, the P2 domain or functional fragment thereof from the other virus replaces one or more of the corresponding residues of the P2 domain of the ORF1 molecule.

In some embodiments, an ORF1 molecule comprises a P1 domain, or a functional fragment thereof, from a hepatitive E virus (HEV) capsid protein, or an amino acid sequence having at least 75%, 80%, 85%, 90%, 95%, 96%, 97%, 98%, or 99% identity thereto. In some embodiments, an ORF1 molecule comprises a P2 domain, or a functional fragment thereof, from an HEV capsid protein, or an amino acid sequence having at least 75%, 80%, 85%, 90%, 95%, 96%, 97%, 98%, or 99% identity thereto.

### Other Domain Mutants

An ORF1 molecule as described herein may comprise one more additional mutations relative to a wild-type ORF1 protein sequence.

In some embodiments, a structural jelly-roll region or portion thereof of a heterologous protein (e.g., a capsid protein from a virus other than an Anellovirus, e.g., as described herein), or a sequence having at least 80%, 85%, 90%, 95%, 96%, 97%, 98%, or 99% identity thereto, is inserted into the sequence of the ORF1 molecule (e.g., at the position of a deletion or truncation of the structural jelly-roll region of the ORF1 molecule). In some embodiments, the structural jelly-roll region of an ORF1 molecule comprises one or more mutations (e.g., at least 2, 3, 4, 5, 6, 7, 8, 9, or 10 mutations) in a beta strand relative to the amino acid sequence of a wild-type Anellovirus ORF1 structural jelly-roll region.

In some embodiments, an C-terminal domain or portion thereof of a heterologous protein (e.g., a capsid protein from a virus other than an Anellovirus, e.g., as described herein), or a sequence having at least 80%, 85%, 90%, 95%, 96%, 97%, 98%, or 99% identity thereto, is inserted into the sequence of the ORF1 molecule (e.g., at the C-terminus and/or at the position of a deletion or truncation of the structural CTD).

### ORF2 Molecules

In some embodiments, the anellovector or anelloVLP comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in Table A1-A32), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in Table A1-A32. In some embodiments, an ORF2 molecule is encoded by an ORF2 nucleic acid. In some embodiments, the ORF2 nucleic acid comprises an antisense strand, which can be directly transcribed to produce mRNA encoding the ORF2 molecule. In some embodiments, the ORF2 nucleic acid comprises a sense strand.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule comprises at least one difference (e.g., a mutation, chemical modification, or epigenetic alteration) relative to a wild-type ORF2 protein, e.g., as described herein (e.g., as shown in Table A1-A32).

### Conserved ORF2 Motif

In some embodiments, a polypeptide (e.g., an ORF2 molecule) described herein comprises the amino acid sequence [W/F]X⁷HX³CX¹ CX⁵H (SEQ ID NO: 949), wherein Xⁿ is a contiguous sequence of any n amino acids. In embodiments, X⁷ indicates a contiguous sequence of any seven amino acids. In some embodiments, X³ indicates a contiguous sequence of any three amino acids. In some embodiments, X¹ indicates any single amino acid. In some embodiments, X⁵ indicates a contiguous sequence of any five amino acids. In some embodiments, the [W/F] can be either tryptophan or phenylalanine. In some embodiments, the [W/F]X⁷HX³CX¹CX⁵H (SEQ ID NO: 949) is comprised within the structural N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein Xⁿ is a contiguous sequence of any n amino acids.

### Genetic Elements

In some embodiments, the Anelloviridae family vector (e.g., anellovector) comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500, 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein.

In some embodiments, the genetic element has at least about 70%, 75%, 80%, 8%, 90%, 95%, 96%, 97%, 98%, 99%, or 100% sequence identity to an Anellovirus nucleic acid sequence, e.g., as described herein (e.g., as described in any of Tables N1-N32), or a fragment thereof, or encodes an amino acid sequence having at least about 70%, 75%, 80%, 8%, 90%, 95%, 96%, 97%, 98%, 99%, or 100% sequence identity to an Anellovirus amino acid sequence (e.g., as described in any of Tables A1-A32), or a fragment thereof. In some embodiments, the genetic element comprises a sequence encoding an effector (e.g., an endogenous effector or an exogenous effector, e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater). In some embodiments, the genetic element has a length of about 2.5-4.6, 2.8-4.0, 3.0-3.8, or 3.2-3.7 kb. In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, an Anellovirus amino acid sequence, e.g., as listed in any of Tables A1-A32.

In some embodiments, the genetic element was produced from a double-stranded circular DNA (e.g., produced by in vitro circularization). In some embodiments, the genetic element was produced by rolling circle replication from the double-stranded circular DNA. In some embodiments, the rolling circle replication occurs in a cell (e.g., a host cell, e.g., a mammalian cell, e.g., a human cell, e.g., a HEK293T cell, an A549 cell, or a Jurkat cell). In some embodiments, the genetic element can be amplified exponentially by rolling circle replication in the cell. In some embodiments, the genetic element can be amplified linearly by rolling circle replication in the cell. In some embodiments, the double-stranded circular DNA or genetic element is capable of yielding at least 2, 4, 8, 16, 32, 64, 128, 256, 518, 1024 or more times the original quantity by rolling circle replication in the cell. In some embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In some embodiments, the double-stranded circular DNA and/or the genetic element does not comprise one or more mammalian plasmid elements (e.g., a mammalian origin of replication or a selectable marker, e.g., a mammalian resistance gene). In some embodiments, the double-stranded circular DNA and/or the genetic element does not comprise a mammalian plasmid backbone. In some embodiments, the double-stranded circular DNA and/or the genetic element does not comprise one or more insect plasmid elements (e.g., an insect origin of replication or a selectable marker, e.g., a insect resistance gene). In some embodiments, the double-stranded circular DNA and/or the genetic element does not comprise an insect plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

In some embodiments, a genetic element as described herein comprises a sequence (e.g., a TATA box, cap site, transcriptional start site, 5′ UTR, open reading frame (ORF), poly(A) signal, or GC-rich region sequence) as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, a genetic element comprises a sequence encoding an effector (e.g., an exogenous effector). In some embodiments, the effector-encoding sequence is inserted into an Anellovirus genome sequence (e.g., as described herein). In some embodiments, the effector-encoding sequence replaces a contiguous sequence (e.g., of at least 5, 10, 15, 20, 25, 30, 40, 50, 60, 70, 80, 90, 100, 200, 300, 400, 500, 600, 700, 800, 900, 1000, or more nucleotides) from the Anellovirus genome sequence. In some embodiments, the effector-encoding sequence replaces a TATA box, cap site, transcriptional start site, 5′ UTR, open reading frame (ORF), poly(A) signal, or GC-rich region sequence, or a portion thereof (e.g., a portion consisting of at least 5, 10, 15, 20, 25, 30, 40, 50, 60, 70, 80, 90, 100, 200, 300, 400, 500, 600, 700, 800, 900, 1000, or more nucleotides), e.g., as described herein, or a sequence having at least 70% 80%, 85%, 90% 95%, 96%, 97%, 98% and 99% nucleotide sequence identity thereto.

In some embodiments, the sequence of a first nucleic acid element comprised in a genetic element (e.g., a TATA box, cap site, transcriptional start site, 5′ UTR, open reading frame (ORF), poly(A) signal, or GC-rich region) overlaps with the sequence of a second nucleic acid element (e.g., a TATA box, cap site, transcriptional start site, 5′ UTR, open reading frame (ORF), poly(A) signal, or GC-rich region), e.g., by at least 5, 10, 15, 20, 25, 30, 40, 50, 60, 70, 80, 90, 100, 150, 200, 250, 300, 400, or 500 nucleotides. In some embodiments, the sequence of a first nucleic acid element comprised in a genetic element (e.g., a TATA box, cap site, transcriptional start site, 5′ UTR, open reading frame (ORF), poly(A) signal, or GC-rich region) does not overlap with the sequence of a second nucleic acid element (e.g., a TATA box, cap site, transcriptional start site, 5′ UTR, open reading frame (ORF), poly(A) signal, or GC-rich region).

### Protein Binding Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77 (24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds a structural arginine-rich region of the substantially non-pathogenic protein. In some embodiments, the genetic element comprises a protein binding sequence as described in Example 8 of PCT Publication No. WO 2020/123816, incorporated by reference herein in its entirety. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence (e.g., as shown in any of Tables N1-N32).

In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of any of Tables N1-N32.

### 5′ UTR Regions

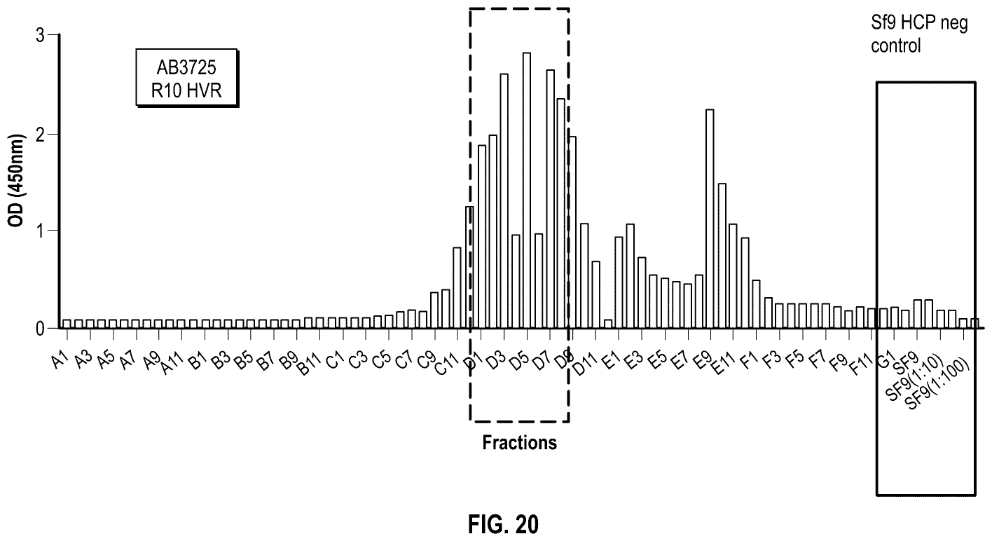

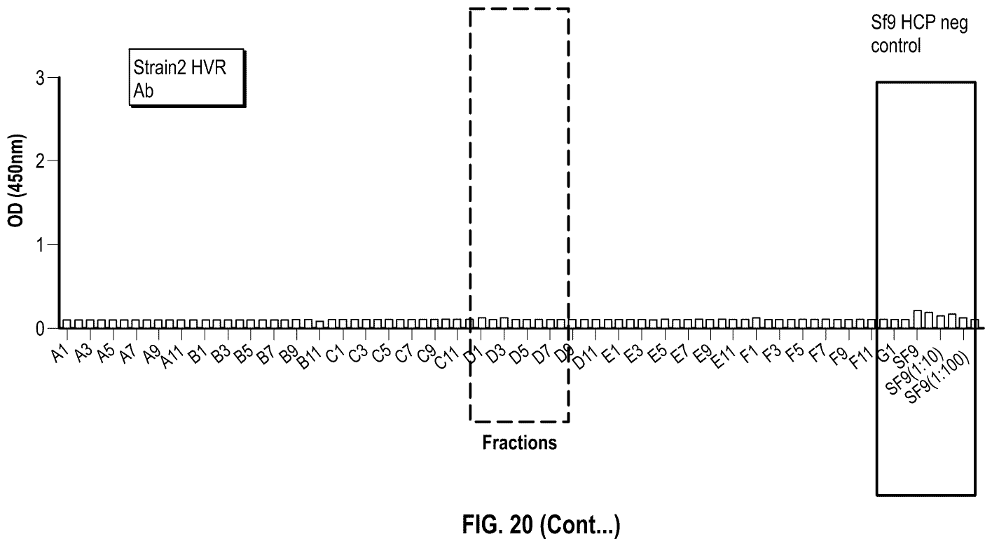

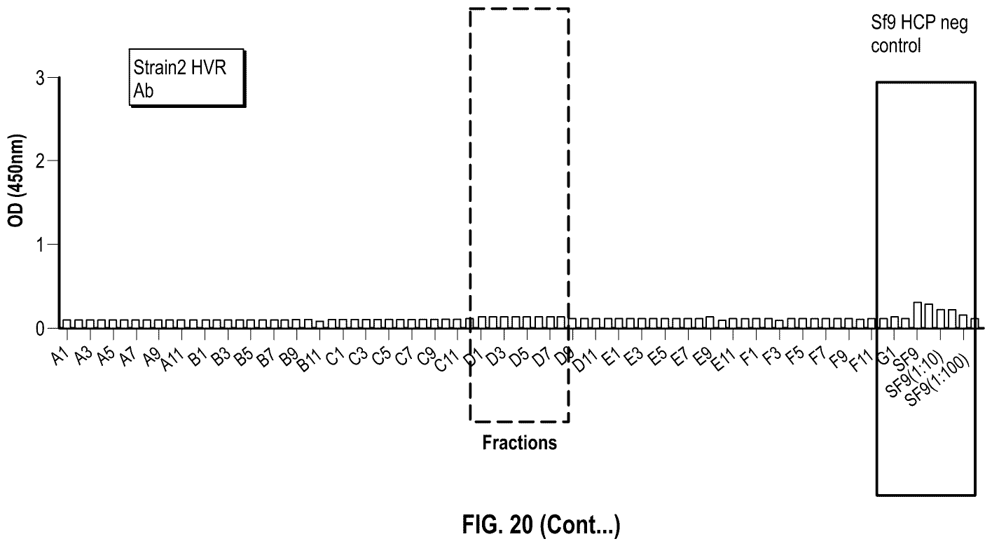

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 38 and/or FIG. 20 of PCT Publication No. WO 2020/123816. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and X₅ are each independently any nucleotide, e.g., wherein X₁=G or T, X₂=C or A, X₃=G or A, X₄=T or C, and X₅=A, C, or T). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJNO2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

In some embodiments, the genetic element comprises a nucleic acid sequence having at least about 70%, 75%, 80%, 85%, 90%, 95%, 96%, 97%, 98%, 99%, or 100% sequence identity to the reverse complement of sequence annotated as the GC-rich domain nucleotide sequence of any of Tables N1-N32.

### 5′ UTR Conserved Domains

A genetic element may include an Anellovirus 5′ UTR conserved domain. Typically, the negative strand comprising the Anellovirus 5′ UTR conserved domain is packaged into a particle (e.g., an Anelloviridae family vector as described herein. In some embodiments, the Anellovirus 5′ UTR conserved domain is a wild-type Anellovirus 5′ UTR conserved domain. In some embodiments, the Anellovirus 5′ UTR conserved domain is an engineered Anellovirus 5′ UTR conserved domain having a nucleic acid sequence with at least one difference relative to the closest wild-type Anellovirus 5′ UTR conserved domain sequence. In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any of Tables N1-N32 or Table 38.

TABLE 38
Exemplary 5′ UTR sequences from Anelloviruses. Note this table provides the
positive strand sequence. It is understood that embodiments described herein
referring to sequences in this table can refer to the positive or negative strand
sequence, e.g., the reverse complement of a sequence described in the table.

| Source | Sequence | SEQ ID NO: |
|---|---|---|
| Consensus | CGGGTGCCGX₁AGGTGAGTTTACACACCGX₂ | 105 |
| | AGTCAAGGGGCAATTCGGGCTCX₃GGACTGG | |
| | CCGGGCX₄X₅TGGG | |
| | X₁ = G or T | |
| | X₂ = C or A | |
| | X₃ = G or A | |
| | X₄ = T or C | |
| | X₅ = A, C, or T | |
| Exemplary TTV | CGGGTGCCGGAGGTGAGTTTACACACCGCA | 106 |
| Sequence | GTCAAGGGGCAATTCGGGCTCGGGACTGGC | |
| | CGGGCTWTGGG | |
| TTV-CT30F | CGGGTGCCGTAGGTGAGTTTACACACCGCA | 107 |
| | GTCAAGGGGCAATTCGGGCTCGGGACTGGC | |
| | CGGGCTATGGG | |
| TTV-HD23a | CGGGTGCCGGAGGTGAGTTTACACACCGCA | 108 |
| | GTCAAGGGGCAATTCGGGCTCGGGACTGGC | |
| | CGGGCCCTGGG | |
| TTV-JA20 | CGGGTGCCGGAGGTGAGTTTACACACCGCA | 109 |
| | GTCAAGGGGCAATTCGGGCTCGGGACTGGC | |
| | CGGGCTTTGGG | |
| TTV-TJN02 | CGGGTGCCGGAGGTGAGTTTACACACCGCA | 110 |
| | GTCAAGGGGCAATTCGGGCTCGGGACTGGC | |
| | CGGGCTATGGG | |
| TTV-tth8 | CGGGTGCCGGAGGTGAGTTTACACACCGAA | 111 |
| | GTCAAGGGGCAATTCGGGCTCAGGACTGGC | |
| | CGGGCTTTGGG | |
| Alphatorquevirus | CGGGTGCCGGAGGTGAGTTTACACACCGCA | 112 |
| Consensus 5′ UTR | GTCAAGGGGCAATTCGGGCTCGGGACTGGC | |
| | CGGGC | |
| | X₁X₂TGGG; wherein X₁ comprises T or C, and | |
| | wherein X₂ comprises A, C, or T. | |
| Alphatorquevirus | CGGGTGCCGTAGGTGAGTTTACACACCGCA | 113 |
| Clade 1 5′ UTR (e.g., | GTCAAGGGGCAATTCGGGCTCGGGACTGGC | |
| TTV-CT30F) | CGGGCTATGGG | |
| Alphatorquevirus | CGGGTGCCGGAGGTGAGTTTACACACCGCA | 114 |
| Clade 2 5′ UTR (e.g., | GTCAAGGGGCAATTCGGGCTCGGGACTGGC | |
| TTV-P13-1) | CGGGCCCGGG | |
| Alphatorquevirus | CGGGTGCCGGAGGTGAGTTTACACACCGAA | 115 |
| Clade 3 5′ UTR (e.g., | GTCAAGGGGCAATTCGGGCTCAGGACTGGC | |
| TTV-tth8) | CGGGCTTTGGG | |
| Alphatorquevirus | CGGGTGCCGGAGGTGAGTTTACACACCGCA | 116 |
| Clade 4 5′ UTR (e.g., | GTCAAGGGGCAATTCGGGCTCGGGAGGCCG | |
| TTV-HD20a) | GGCCATGGG | |
| Alphatorquevirus | CGGGTGCCGGAGGTGAGTTTACACACCGCA | 117 |
| Clade 5 5′ UTR (e.g., | GTCAAGGGGCAATTCGGGCTCGGGACTGGC | |
| TTV-16) | CGGGCCCCGGG | |
| Alphatorquevirus | CGGGTGCCGGAGGTGAGTTTACACACCGCA | 118 |
| Clade 6 5′ UTR (e.g., | GTCAAGGGGCAATTCGGGCTCGGGACTGGC | |
| TTV-TJN02) | CGGGCTATGGG | |
| Alphatorquevirus | CGGGTGCCGAAGGTGAGTTTACACACCGCA | 119 |
| Clade 7 5′ UTR (e.g., | GTCAAGGGGCAATTCGGGCTCGGGACTGGC | |
| TTV-HD16d) | CGGGCTATGGG | |

### Identification of 5′ UTR Sequences

In some embodiments, an Anelloviridae family virus (e.g., Anellovirus) 5′ UTR sequence can be identified within the genome of an Anelloviridae family virus (e.g., Anellovirus) (e.g., a putative Anelloviridae family virus genome identified, for example, by nucleic acid sequencing techniques, e.g., deep sequencing techniques). In some embodiments, an Anelloviridae family virus (e.g., Anellovirus) 5′ UTR sequence is identified by one or both of the following steps:

(i) Identification of circularization junction point: In some embodiments, a 5′ UTR will be positioned near a circularization junction point of a full-length, circularized Anelloviridae family virus (e.g., Anellovirus) genome. A circularization junction point can be identified, for example, by identifying overlapping regions of the sequence. In some embodiments, an overlapping region of the sequence can be trimmed from the sequence to produce a full-length Anelloviridae family virus (e.g., Anellovirus) genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anelloviridae family virus (e.g., Anellovirus) genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anelloviridae family virus (e.g., Anellovirus), e.g., as described herein).

(ii) Identification of 5′ UTR sequence: Once a putative Anelloviridae family virus (e.g., Anellovirus) genome sequence has been obtained, the sequence (or portions thereof, e.g., having a length between about 40-50, 50-60, 60-70, 70-80, 80-90, or 90-100 nucleotides) can be compared to one or more Anelloviridae family virus (e.g., Anellovirus) 5′ UTR sequences (e.g., as described herein) to identify sequences having substantial homology thereto. In some embodiments, a putative Anelloviridae family virus (e.g., Anellovirus) 5′ UTR region has at least 50%, 60%, 70%, 75%, 80%, 85%, 90%, 95%, 96%, 97%, 98%, 99%, or 100% sequence identity to an Anelloviridae family virus (e.g., Anellovirus) 5′ UTR sequence as described herein.

### GC-Rich Regions

A genetic element may include an Anellovirus GC-rich region. Typically, the negative strand comprising the Anellovirus GC-rich region is packaged into a particle (e.g., an Anelloviridae family vector as described herein. In some embodiments, the Anellovirus GC-rich region is a wild-type Anellovirus GC-rich region. In some embodiments, the Anellovirus GC-rich region is an engineered Anellovirus GC-rich region having a nucleic acid sequence with at least one difference relative to the closest wild-type Anellovirus GC-rich region sequence. In some embodiments, the Anellovirus GC-rich region comprises a contiguous sequence of at least 20, 25, 30, 31, 32, 33, 34, 35, 36, 37, 38, 39, or 40 consecutive nucleotides having a GC content of at least 70%, 75%, 80%, 85%, 90%, 95%, or 99%. In some embodiments, the genetic element (e.g., protein-binding sequence of the genetic element) comprises a nucleic acid sequence having at least about 70%, 75%, 80%, 85%, 90%, 95%, 96%, 97%, 98%, 99%, or 100% sequence identity to a GC-rich sequence shown inany of Tables N1-N32 or Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In some embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJNO2, or TTV-HD16d, e.g., as listed in Table 39. In some embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 105, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJNO2, or TTV-HD16d, e.g., as listed in Table 39.

In some embodiments, the 36-nucleotide GC-rich sequence is selected from:

(i)
(SEQ ID NO: 160)
CGCGCTGCGCGCGCCGCCCAGTAGGGGGAGCCATGC,
(ii)
(SEQ ID NO: 164)
GCGCTX₁CGCGCGCGCGCCGGGGGGCTGCGCCCCCCC,

wherein X₁ is selected from T, G, or A;

(iii)
(SEQ ID NO: 165)
GCGCTTCGCGCGCCGCCCACTAGGGGGCGTTGCGCG;
(iv)
(SEQ ID NO: 166)
GCGCTGCGCGCGCCGCCCAGTAGGGGGCGCAATGCG;
(v)
(SEQ ID NO: 167)
GCGCTGCGCGCGCGGCCCCCGGGGGAGGCATTGCCT;
(vi)
(SEQ ID NO: 168)
GCGCTGCGCGCGCGCGCCGGGGGGGCGCCAGCGCCC;
(vii)
(SEQ ID NO: 169)
GCGCTTCGCGCGCGCGCCGGGGGGCTCCGCCCCCCC;
(viii)
(SEQ ID NO: 170)
GCGCTTCGCGCGCGCGCCGGGGGGCTGCGCCCCCCC;
(ix)
(SEQ ID NO: 171)
GCGCTACGCGCGCGCGCCGGGGGGCTGCGCCCCCCC;
or
(x)
(SEQ ID NO: 172)
GCGCTACGCGCGCGCGCCGGGGGGCTCTGCCCCCCC.

In some embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In some embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃₀, and X₃₃ are each independently any nucleotide and wherein X₂, X₃, X₈, X₉, X₁₀, X₁₁, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJNO2 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In some embodiments, the genetic element (e.g., protein-binding sequence ofthe genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

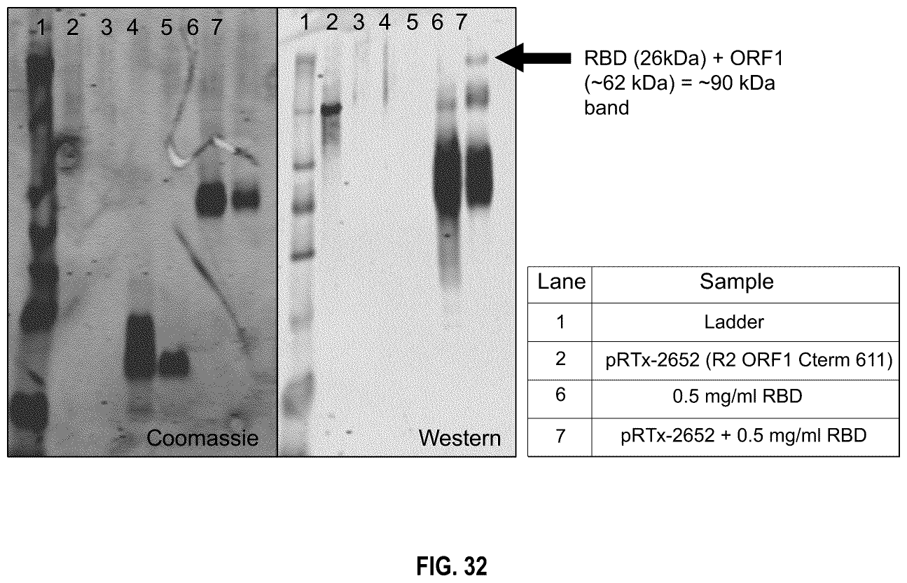

TABLE 39
Exemplary GC-rich sequences from Anelloviruses. Note that this table
provides the positive strand sequence. It is understood that embodiments
described herein referring to sequences in this table can refer to the
positive or negative strand sequence, e.g., the reverse complement of a
sequence described in the table.

| | | | SEQ ID |
|---|---|---|---|
| Source | Sequence | | NO: |
| Consensus | CGGCGGX₁GGX₂GX₃X₄X₅CGCGCTX₆CGC | | 120 |
| | GCGCX7X₈X₉X₁₀CX₁₁X₁₂X₁₃X₁₄GGGGX₁₅X₁₆ | | |
| | X₁₇X₁₈X₁₉X₂₀X₂₁GCX₂₂X₂₃X₂₄X₂₅CCCCCCC | | |
| | X₂₆CGCGCATX₂₇X₂₈GCX₂₉CGGGX₃₀CCCC | | |
| | CCCCCX₃₁X₃₂X₃₃GGGGGGCTCCGX₃₄CCC | | |
| | CCCGGCCCCCC | | |
| | X₁ = G or C | | |
| | X₂ = G, C, or absent | | |
| | X₃ = C or absent | | |
| | X₄ = G or C | | |
| | X₅ = G or C | | |
| | X₆ = T, G, or A | | |
| | X₇ = G or C | | |
| | X₈ = G or absent | | |
| | X₉ = C or absent | | |
| | X₁₀ = C or absent | | |
| | X₁₁ = G, A, or absent | | |
| | X₁₂ = G or C | | |
| | X₁₃ = C or T | | |
| | X₁₄ = G or A | | |
| | X₁₅ = G or A | | |
| | X₁₆ = A, G, T, or absent | | |
| | X₁₇ = G, C, or absent | | |
| | X₁₈ = G, C, or absent | | |
| | X₁₉ = C, A, or absent | | |
| | X₂₀ = C or A | | |
| | X₂₁ = T or A | | |
| | X₂₂ = G or C | | |
| | X₂₃ = G, T, or absent | | |
| | X₂₄ = C or absent | | |
| | X₂₅ = G, C, or absent | | |
| | X₂₆ = G or C | | |
| | X₂₇ = G or absent | | |
| | X₂₈ = C or absent | | |
| | X₂₉ = G or A | | |
| | X₃₀ = G or T | | |
| | X₃₁ = C, T, or absent | | |
| | X₃₂ = G, C, A, or absent | | |
| | X₃₃ = G or C | | |
| | X₃₄ = C or absent | | |
| Exemplary TTV | Full sequence | GCCGCCGCGGCGGCGGSGGNGNSGCGC | 121 |
| Sequence | | GCTDCGCGCGCSNNNCRCCRGGGGGNN | |
| | | NNCWGCSNCNCCCCCCCCCGCGCATGC | |
| | | GCGGGKCCCCCCCCCNNCGGGGGGCTC | |
| | | CGCCCCCCGGCCCCCCCCCGTGCTAAA | |
| | | CCCACCGCGCATGCGCGACCACGCCCC | |
| | | CGCCGCC | |
| | Fragment 1 | GCCGCCGCGGCGGCGGSGGNGNSGCGC | 122 |
| | | GCTDCGCGCGCSNNNCRCCRGGGGGNN | |
| | | NNCWGCSNCNCCCCCCCCCGCGCAT | |
| | Fragment 2 | GCGCGGGKCCCCCCCCCNNCGGGGGGC | 123 |
| | | TCCG | |
| | Fragment 3 | CCCCCCGGCCCCCCCCCGTGCTAAACCC | 124 |
| | | ACCGCGCATGCGCGACCACGCCCCCGC | |
| | | CGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCC | |
| | | CCCGTGCTAAACCCACCGCGCATGCGC | |
| | | GACCACGCCCCCGCCGCC | |
| | Fragment 1 | GCGGCGG | |
| | Fragment 2 | GGGGGCG | |
| | Fragment 3 | GCCGCG | |
| | Fragment 4 | TTCGCGCGCCGCCCACCAGGGGGTG | 129 |
| | Fragment 5 | CTGCG | |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCC | 133 |
| | | CCCGTGCTAAACCCACCGCGCATGCGC | |
| | | GACCACGCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGC | |
| | | TCCG CCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGCGCCAGCG | 137 |
| | Fragment 4 | CCCCCCCCCCCGCGCAT | 138 |
| | Fragment 5 | GCACGGGTCCCCCCCCCCACGGGGGGC | 139 |
| | | TCCG | |
| | Fragment 6 | CCCCCCGGCCCCCCCCC | 140 |
| TTV-JA20 | Full sequence | CCGTCGGCGGGGGGGCCGCGCGCTGCG | 141 |
| | | CGCGCGGCCC- | |
| | | CCGGGGGAGGCACAGCCTCCCCCCCCC | |
| | | GCGCGCATGCGCGCGGGTCCCCCCCCC | |
| | | TCCGGGGGGCTCCGCCCCCCGGCCCCC | |
| | | CCC | |
| | Fragment 1 | CCGTCGGCGGGGGGGCCGCGCGCTGCG | 142 |
| | | CGCGCGGCCC | |
| | Fragment 2 | CCGGGGGAGGCACAGCCTCCCCCCCCC | 143 |
| | | GCGCGCATGCGCGCGGGTCCCCCCCCC | |
| | | TCCGGGGGGCTCCGCCCCCCGGCCCCC | |
| | | CCC | |
| TTV-TJN02 | Full sequence | CGGCGGCGGCG- | 144 |
| | | CGCGCGCTACGCGCGCG--- | |
| | | CGCCGGGGGG----CTGCCGC- | |
| | | CCCCCCCCCGCGCAT | |
| | | GCGCGGGGCCCCCCCCC- | |
| | | GCGGGGGGCTCCG | |
| | | CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTA | |
| | | GGGGGAGCCATGCG--- | |
| | | CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTA | 155 |
| | | GGGGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGG | 160 |
| | | GAGCCATGC | |
| | Fragment 8 | CCGCCATCTTAAGTAGTTGAGGCGGAC | 161 |
| | | GGTGGCGTGAGTTCAAAGGTCACCATC | |
| | | AGCCACACCTACTCAAAATGGTGG | |
| | Fragment 9 | CTTAAGTAGTTGAGGCGGACGGTGGCG | 162 |
| | | TGAGTTCAAAGGTCACCATCAGCCACA | |
| | | CCTACTCAAAATGGTGGACAATTTCTTC | |
| | | CGGGTCAAAGGTTACAGCCGCCATGTT | |
| | | AAAACACGTGACGTATGACGTCACGGC | |
| | | CGCCATTTTGTGACACAAGATGGCCGA | |
| | | CTTCCTTCC | |
| Additional GC- | 36-nucleotide | CGCGCTGCGCGCGCCGCCCAGTAGGGG | 163 |
| rich Sequences (as | consensus GC- | GAGCCATGC | |
| shown in FIG. 32 | rich region | | |
| of PCT Publication | sequence 1 | | |
| No. WO | 36-nucleotide | GCGCTX₁CGCGCGCGCGCCGGGGGGCT | 164 |
| 2020/123816) | region | GCGCCCCCCC, wherein X₁ is selected | |
| | consensus | from T, G, or A | |
| | sequence 2 | | |
| | TTV Clade 1 | GCGCTTCGCGCGCCGCCCACTAGGGGG | 165 |
| | 36-nucleotide | CGTTGCGCG | |
| | region | | |
| | TTV Clade 3 | GCGCTGCGCGCGCCGCCCAGTAGGGGG | 166 |
| | 36-nucleotide | CGCAATGCG | |
| | region | | |
| | TTV Clade 3 | GCGCTGCGCGCGCGGCCCCCGGGGGAG | 167 |
| | isolate GH1 | GCATTGCCT | |
| | 36-nucleotide | | |
| | region | | |
| | TTV Clade 3 | GCGCTGCGCGCGCGCGCCGGGGGGGCG | 168 |
| | sle 1932 36- | CCAGCGCCC | |
| | nucleotide | | |
| | region | | |
| | TTV Clade 4 | GCGCTTCGCGCGCGCGCCGGGGGGCTC | 169 |
| | ctdc002 36- | CGCCCCCCC | |
| | nucleotide | | |
| | region | | |
| | TTV Clade 5 | GCGCTTCGCGCGCGCGCCGGGGGGCTG | 170 |
| | 36-nucleotide | CGCCCCCCC | |
| | region | | |
| | TTV Clade 6 | GCGCTACGCGCGCGCGCCGGGGGGCTG | 171 |
| | 36-nucleotide | CGCCCCCCC | |
| | region | | |
| | TTV Clade 7 | GCGCTACGCGCGCGCGCCGGGGGGCTC | 172 |
| | 36-nucleotide | TGCCCCCCC | |
| | region | | |
| Additional | TTV-CT30F | GCGGCGGGGGGGCGGCCGCGTTCGCGC | 801 |
| Alphatorquevirus | | GCCGCCCACCAGGGGGTGCTGCGCGCC | |
| GC-rich region | | CCCCCCCGCGCATGCGCGGGGCCCCCC | |
| sequences | | CCCGGGGGGGCTCCGCCCCCCCGGCCC | |
| | | CCCCCCGTGCTAAACCCACCGCGCATG | |
| | | CGCGACCACGCCCCCGCCGCC | |
| | TTV-P13-1 | CCGAGCGTTAGCGAGGAGTGCGACCCT | 802 |
| | | ACCCCCTGGGCCCACTTCTTCGGAGCCG | |
| | | CGCGCTACGCCTTCGGCTGCGCGCGGC | |
| | | ACCTCAGACCCCCGCTCGTGCTGACAC | |
| | | GCTTGCGCGTGTCAGACCACTTCGGGCT | |
| | | CGCGGGGGTCGGG | |
| | TTV-tth8 | GCCGCCGCGGCGGCGGGGGGCGGCGCG | 803 |
| | | CTGCGCGCGCCGCCCAGTAGGGGGAGC | |
| | | CATGCGCCCCCCCCCGCGCATGCGCGG | |
| | | GGCCCCCCCCCGCGGGGGGCTCCGCCC | |
| | | CCCGGCCCCCCCCG | |
| | TTV-HD20a | CGGCCCAGCGGCGGCGCGCGCGCTTCG | 804 |
| | | CGCGCGCGCCGGGGGGCTCCGCCCCCC | |
| | | CCCGCGCATGCGCGGGGCCCCCCCCCG | |
| | | CGGGGGGCTCCGCCCCCCGGTCCCCCC | |
| | | CCG | |
| | TTV-16 | CGGCCGTGCGGCGGCGCGCGCGCTTCG | 805 |
| | | CGCGCGCGCCGGGGGCTGCCGCCCCCC | |
| | | CCCGCGCATGCGCGCGGGGCCCCCCCC | |
| | | CGCGGGGGGCTCCGCCCCCCGGCCCCC | |
| | | CCCCCCG | |
| | TTV-TJN02 | CGGCGGCGGCGCGCGCGCTACGCGCGC | 806 |
| | | GCGCCGGGGGGCTGCCGCCCCCCCCCC | |
| | | GCGCATGCGCGGGGCCCCCCCCCGCGG | |
| | | GGGGCTCCGCCCCCCGGCCCCCC | |
| | TTV-HD16d | GGCGGCGGCGCGCGCGCTACGCGCGCG | 807 |
| | | CGCCGGGGAGCTCTGCCCCCCCCCGCG | |
| | | CATGCGCGCGGGTCCCCCCCCCGCGGG | |
| | | GGGCTCCGCCCCCCGGTCCCCCCCCCG | |

In some embodiments, the genetic element comprises a nucleic acid sequence having at least about 70%, 75%, 80%, 85%, 90%, 95%, 96%, 97%, 98%, 99%, or 100% sequence identity to the Anellovirus GC-rich nucleotide sequence of any one of Tables N1-N32.

### Effectors

In some embodiments, the genetic element may include one or more sequences that encode a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described in Example 10, 12, or 22 of PCT Publication No. WO 2020/123816, incorporated by reference herein in its entirety. In some embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In some embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF as described herein, e.g., an ORF1, ORF1/1, ORF1/2, ORF2, ORF2/2, ORF2/3, and/or ORF2t/3 as shown in any one of Tables A1-A32 or N1-N32).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload.

### Regulatory Nucleic Acid

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In some embodiments, the regulatory nucleic acid encodes an miRNA.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector or anelloVLP may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic, e.g., a wild-type protein or a functional fragment or variant thereof. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

In some embodiments, an effector as described herein comprises a cytosolic polypeptide or cytosolic peptide, e.g., a wild-type protein or a functional fragment or variant thereof.

In some embodiments, the effector comprises a regulatory intracellular polypeptide, e.g., a wild-type protein or a functional fragment or variant thereof. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

In some embodiments, an effector as described herein comprises a secreted polypeptide effector, e.g., a wild-type protein or a functional fragment or variant thereof. Exemplary secreted therapeutics include cytokines and cytokine receptors.

Exemplary cytokines and cytokine receptors are described, e.g., in Akdis et al., “Interleukins (from IL-1 to IL-38), interferons, transforming growth factor f, and TNF-α: Receptors, functions, and roles in diseases” October 2016 Volume 138, Issue 4, Pages 984-1010.

Additional exemplary secreted therapeutics include polypeptide hormones and receptors, e.g., a wild-type protein or a functional fragment or variant thereof.

Additional exemplary secreted therapeutics include growth factors, e.g., a wild-type protein or a functional fragment or variant thereof. Exemplary growth factors and growth factor receptors are described, e.g., in Bafico et al., “Classification of Growth Factors and Their Receptors” Holland-Frei Cancer Medicine. 6th edition.

Additional exemplary secreted therapeutics include clotting-associated factors, e.g., a wild-type protein or a functional fragment or variant thereof.

In some embodiments, an effector described herein comprises a protein replacement therapeutic, e.g., a wild-type protein or a functional fragment or variant thereof. Exemplary protein replacement therapeutics are described herein.

In some embodiments, an effector described herein comprises an enzymatic effector, e.g., a wild-type protein or a functional fragment or variant thereof.

In some embodiments, an effector described herein comprises a non-enzymatic effector, e.g., a wild-type protein or a functional fragment or variant thereof.

In some embodiments, an effector described herein comprises a protein that, when mutated, causes a lysosomal storage disorder, e.g., a wild-type protein or a functional fragment or variant thereof. In some embodiments, an effector described herein comprises a transporter protein, e.g., a wild-type protein or a functional fragment or variant thereof.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polyeptpide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

In some embodiments, an effector as described herein comprises a transformation factor, e.g., a wild-type protein or a fragment or variant thereof. In embodiments, the transformation factor is a protein factor that transforms fibroblasts into differentiated cells. In some embodiments, an effector as described herein comprises a protein that stimulates cellular regeneration, e.g., a wild-type protein or a fragment or variant thereof.

In some embodiments, an effector as described herein modulates STING/cGAS signaling, e.g., a wild-type protein or a fragment or variant thereof. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7 (283): 283ra52. Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition, anellovector, or anelloVLP described herein includes a polypeptide linked to a ligand that is capable of targeting a specific location, tissue, or cell.

### Gene Editing Components

The genetic element of the anellovector may include one or more genes that encode a component of a gene editing system. Alternatively, an anellovector or anelloVLP as described herein may comprise a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7 (2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et a1, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al, Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the anellovector, e.g., synthetic anellovector, may include sequences that encode one or more replication proteins. In some embodiments, the anellovector may replicate by a rolling-circle replication method, e.g., synthesis of the leading strand and the lagging strand is uncoupled. In such embodiments, the anellovector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

### Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

### Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more.

In some embodiments, the genetic element comprises a nucleotide sequence with at least about 75% nucleotide sequence identity, at least about 80%, 85%, 90% 95%, 96%, 97%, 98%, 99% or 100% nucleotide sequence identity to any one of the nucleotide sequences described herein, e.g., as listed in any one of Tables N1-N32. Since the genetic code is degenerate, a homologous nucleotide sequence can include any number of silent base changes, i.e., nucleotide substitutions that nonetheless encode the same amino acid.

### Gene Editing Component

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7 (2013):397-405; CRISPR methods of gene editing are described, e.g., in Guan et al., Application of CRISPR-Cas system in gene therapy: Pre-clinical progress in animal model. DNA Repair 2016 October; 46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas9 generates only a single-strand break; a catalytically inactive Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence. Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

As used herein, a “biologically active portion of an effector domain” is a portion that maintains the function (e.g. completely, partially, or minimally) of an effector domain (e.g., a “minimal” or “core” domain). In some embodiments, the anellovector includes a gene encoding a fusion of a dCas9 with all or a portion of one or more effector domains to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Proteinaceous Exterior

In some embodiments, the anellovector, e.g., synthetic anellovector, comprises a proteinaceous exterior that encloses the genetic element. In some embodiments, the anelloVLP, e.g., synthetic anelloVLP, comprises a proteinaceous exterior and an effector (e.g., an exogenous effector). The proteinaceous exterior can comprise a substantially non-pathogenic exterior protein that fails to elicit an unwanted immune response in a mammal. The proteinaceous exterior of the anellovectors or anelloVLPs typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, a structural arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 sequence or a capsid protein sequence as listed in any one of Tables A1-A32. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to any one of the nucleotide sequences described herein, e.g., an Anellovirus capsid sequence or a capsid protein sequence as listed in any one of Tables A1-A32. In some embodiments, the protein comprises a capsid protein or a functional fragment of a capsid protein that is encoded by a capsid nucleotide sequence or a sequence having at least about 60%, 65%, 70%, 75%, 80%, 85%, 90% 95%, 96%, 97%, 98%, 99%, or 100% nucleotide sequence identity to any one of the nucleotide sequences described herein, e.g., an Anellovirus capsid sequence or a capsid protein sequence as listed in any one of Tables N1-N32.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to any one of the amino acid sequences described herein, e.g., an Anellovirus capsid sequence or a capsid protein sequence in any one of Tables A1-A32. In some embodiments, the anellovector comprises a nucleotide sequence encoding a capsid protein or a functional fragment of a capsid protein or a sequence having at least about 60%, 65%, 70%, 75%, 80%, 85%, 90% 95%, 96%, 97%, 98%, 99%, or 100% sequence identity to any one of the amino acid sequences described herein, e.g., an Anellovirus capsid sequence or a capsid protein sequence in any one of Tables A1-A32.

In some embodiments, the anellovector comprises a nucleotide sequence encoding an amino acid sequence having about position 1 to about position 150 (e.g., or any subset of amino acids within each range, e.g., about position 20 to about position 35, about position 25 to about position 30, about position 26 to about 30), about position 150 to about position 390 (e.g., or any subset of amino acids within each range, e.g., about position 200 to about position 380, about position 205 to about position 375, about position 205 to about 371), about 390 to about position 525, about position 525 to about position 850 (e.g., or any subset of amino acids within each range, e.g., about position 530 to about position 840, about position 545 to about position 830, about position 550 to about 820), about 850 to about position 950 (e.g., or any subset of amino acids within each range, e.g., about position 860 to about position 940, about position 870 to about position 930, about position 880 to about 923) of the amino acid sequences described herein, an Anellovirus amino acid sequence, e.g., as listed in any one of Tables A1-A32, or a functional fragment thereof. In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to about position 1 to about position 150 (e.g., or any subset of amino acids within each range as described herein), about position 150 to about position 390, about position 390 to about position 525, about position 525 to about position 850, about position 850 to about position 950 of the amino acid sequences described herein, an Anellovirus amino acid sequence, e.g., as listed in any one of Tables A1-A32.

In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences or ranges of amino acids described herein, an Anellovirus amino acid sequence, e.g., as listed in any one of Tables A1-A32. In some embodiments, the ranges of amino acids with less sequence identity may provide one or more of the properties described herein and differences in cell/tissue/species specificity (e.g. tropism).

In some embodiments, the anellovector or anelloVLP lacks lipids in the proteinaceous exterior. In some embodiments, the anellovector or anelloVLP lacks a lipid bilayer, e.g., a viral envelope. In some embodiments, the interior of the anellovector or anelloVLP is entirely covered (e.g., 100% coverage) by a proteinaceous exterior. In some embodiments, the interior of the anellovector or anelloVLP is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, e.g., so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: one or more glycosylated proteins, a hydrophilic DNA-binding region, a structural arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### Surface Moieties

An anellovector or anelloVLP as described herein may, in some instances, include one or more moieties attached to its surface (e.g., a surface moiety that can act as an effector and/or a targeting agent). In some instances, an anellovector or anelloVLP comprises more than one distinct surface moiety (e.g., a first surface moiety having an effector function as described herein and a second surface moiety that targets the anellovector or anelloVLP to a cell or tissue of interest). In some instances, the surface moiety is covalently attached to the surface of the anellovector or anelloVLP. For example, the surface moiety may be covalently attached to the proteinaceous exterior or a component thereof (e.g., covalently attached to an ORF1 molecule of the proteinaceous exterior). In certain embodiments, the surface moiety is fused to an ORF1 molecule. In some instances, the surface moiety is noncovalently attached to the surface of the anellovector or anelloVLP. For example, the surface moiety may be noncovalently bound to the proteinaceous exterior or a component thereof (e.g., noncovalently bound to an ORF1 molecule of the proteinaceous exterior). In certain embodiments, the surface moiety comprises a region that specifically binds to a cognate moiety on or attached to the ORF1 molecule. In an embodiment, the ORF1 molecule comprises a binding moiety (e.g., an antibody molecule) that specifically recognizes an epitope on the region on the surface moiety. In an embodiment, the surface moiety comprises a binding moiety (e.g., an antibody molecule) that specifically recognizes an epitope on the ORF1 molecule. In an embodiment, the surface moiety comprises a streptavidin moiety that binds to a biotin moiety on the surface of the anellovector or anelloVLP (e.g., a biotin moiety attached to an ORF1 molecule of the proteinaceous exterior of the anellovector or anelloVLP). In an embodiment, the surface moiety comprises a biotin moiety that binds to a streptavidin moiety on the surface of the anellovector or anelloVLP (e.g., a streptavidin moiety attached to an ORF1 molecule of the proteinaceous exterior of the anellovector or anelloVLP).

In embodiments, all copies of an ORF1 molecule in the proteinaceous exterior of an anellovector or anelloVLP are attached to copies of the surface moiety. In embodiments, some copies of the ORF1 molecule in the proteinaceous exterior of an anellovector or anelloVLP are attached to copies of the surface moiety and some copies of the ORF1 molecule in the proteinaceous exterior of an anellovector or anelloVLP are not attached to copies of the surface moiety. In embodiments, some copies of the ORF1 molecule in the proteinaceous exterior of an anellovector or anelloVLP are attached to copies of a first surface moiety and some copies of the ORF1 molecule in the proteinaceous exterior of an anellovector or anelloVLP are attached to copies of a second surface moiety. In embodiments, some copies of the ORF1 molecule in the proteinaceous exterior of an anellovector or anelloVLP are attached to copies of a third surface moiety.

In some embodiments, all copies of an ORF1 molecule in the proteinaceous exterior of an anellovector or anelloVLP are attached at the same position (e.g., a lysine residue) of the ORF1 molecule to a copy of a surface moiety. In some embodiments, a first copy of an ORF1 molecule in the proteinaceous exterior of an anellovector or anelloVLP is attached at a first position (e.g., a first lysine residue) of the ORF1 molecule to a first copy of a surface moiety, and a second copy of an ORF1 molecule in the proteinaceous exterior of an anellovector or anelloVLP is attached at a second position (e.g., a second lysine residue) of the ORF1 molecule to a second of a surface moiety. In some embodiments, the proteinaceous exterior further comprises one or more copies of an ORF1 molecule having a surface moiety attached to a one or more additional positions (e.g., one or more additional lysine residues). In certain embodiments, the first lysine residue, the second lysine residue, and/or the one or more additional lysine residues are positioned on the surface of the proteinaceous exterior. In some embodiments, the surface moiety is attached to the ORF1 molecule via click chemistry or genetic grafting, e.g., as described herein.

In some instances, the surface moiety comprises an effector function (e.g., as described herein). For example, the surface moiety may modulate a biological activity, e.g., of a target cell or organ. In some instances, the surface moiety induces modulation of the biological activity via binding to a cognate moiety on a target cell. For example, the surface moiety may comprise a ligand that binds to a receptor on the surface of the target cell, e.g., wherein binding of the surface moiety to the receptor initiates a downstream signaling cascade of interest. In some instances, the effector activity comprises increasing or decreasing enzymatic activity, gene expression, cell signaling, and/or cellular or organ function within a target cell or organ. Effector activities may also include binding regulatory proteins to modulate activity of the regulator, such as transcription or translation. Effector activities also may include activator or inhibitor functions.

In some instances, the surface moiety can target the anellovector or anelloVLP to a target cell. For example, the surface moiety may specifically bind to a cognate moiety on the surface of the target cell. The cognate moiety on the surface of the target cell may be, for example, a molecule specifically expressed or preferentially expressed by the target cell. The cognate moiety may be, for example, a polypeptide, lipid, sugar, or small molecule. In certain embodiments, the cognate moiety is a transmembrane protein (e.g., comprising an extracellular domain that binds to the surface moiety of the anellovector or the anelloVLP). In certain embodiments, the cognate moiety is tethered to the surface of the cell (e.g., via a GPI anchor). In some instances, the surface moiety provides a tropism (e.g., to a target tissue or target cell type) for the anellovector or anelloVLP.

In some instances, the surface moiety comprises an effector function and a targeting function, e.g., as described herein. In some instances, the surface moiety comprises a domain having an effector function as described herein. In some instances, the surface moiety comprises a domain having a targeting function as described herein.

In some instances, the surface moiety binds specifically to one cognate moiety. In some embodiments, the surface moiety binds specifically to more than one cognate moiety. In some embodiments, the surface moiety comprises a plurality of binding regions, for example, each of which specifically binds to a different cognate moiety. For example, the surface moiety may be bispecific or trispecific. In some embodiments, the surface moiety comprises a plurality of binding regions, for example, each of which binds to the same cognate moiety or copies thereof (e.g., at different epitopes of the cognate moiety or the same epitope of the cognate moiety). In certain embodiments, the surface moiety having multiple binding regions that specifically bind to the same cognate moiety results in greater avidity for the target moiety.

### Click Chemistry

In an aspect, the disclosure provides an ORF1 molecule comprising: (i) the amino acid sequence of an Anellovirus ORF1 protein, or an amino acid sequence having at least 75%, 80%, 85%, 90%, 95%, 96%, 97%, 98%, or 99% sequence identity thereto; and (ii) a click handle (e.g., an NHS click handle or a maleimide click handle, e.g., as described herein). In certain embodiments, the click handle is covalently attached to the ORF1 molecule. In certain embodiments, the click handle is noncovalently attached to the ORF1 molecule. In certain embodiments, the click handle is used to attach the ORF1 molecule to a surface moiety, e.g., via a click reaction, e.g., as described herein.

A “click handle,” as that term is used herein, refers to a chemical moiety that is capable of reacting with a second click handle in a click reaction. In some embodiments, a click handle comprises an NHS moiety and/or a maleimide moiety. In certain embodiments, a click handle comprises a DBCO moiety. In certain embodiments, a click handle comprises an azide moiety. In some embodiments, a click handle is attached to a polypeptide (e.g., an ORF1 molecule). In other embodiments, a click handle comprises a reactive group capable of forming a covalent bond with a polypeptide (e.g., an ORF1 molecule). A “click reaction”, as that term is used herein, refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic byproducts that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In embodiments, a click reaction has a second order forward rate constant of 10-200 M-1s-1, 1-20 M-1s-1, or at least 1, 2, 3, 5, 10, 20, 50, 60, 100, 200, 500, 1E3, 2E3, 5E3, 1E4, 2E4, 5E4, 1E5, 2E5, 5E5, or 1E6 M-1s-1, e.g., at 20° C. in PBS. In some embodiments, a click reaction has at least 75%, 80%, 85%, 90%, 95%, 96%, 97%, 98%, or 99% yield, e.g., for a reaction time of 1 hour at 20° C. in PBS.

In some embodiments, a surface moiety is attached to a polypeptide by conjugation of a lysine residue on the surface of the polypeptide to an NHS click handle on the surface moiety (e.g., as described in Example 2 or 3 herein). In some embodiments, a surface moiety is attached to a polypeptide by conjugation of a lysine residue on the surface of the surface moiety to an NHS click handle on the polypeptide (e.g., as described in Example 2 or 3 herein).

In some embodiments, a surface moiety is attached to a polypeptide by conjugation of a cysteine residue on the surface of the polypeptide to a maleimide click handle on the surface moiety (e.g., as described in Example 4 herein). In some embodiments, a surface moiety is attached to a polypeptide by conjugation of a cysteine residue on the surface of the surface moiety to a maleimide click handle on the polypeptide (e.g., as described in Example 4 herein).

In an aspect, the disclosure provides an ORF1 molecule comprising a surface moiety, wherein the surface moiety was attached to the ORF1 molecule via a click reaction.

In an aspect, the disclosure provides a particle (e.g., an anellovector or anelloVLP) comprising: (i) a proteinaceous exterior comprising an ORF1 molecule; and (ii) a click handle (e.g., an NHS click handle and/or a maleimide click handle, e.g., as described herein). In certain embodiments, the click handle is covalently attached to the ORF1 molecule. In some embodiments, the particle is an anellovector comprising a genetic element enclosed in the proteinaceous exterior. In some embodiments, the particle is an anelloVLP comprising an effector (e.g., an exogenous effector), e.g., enclosed in the proteinaceous exterior.

### Mutations of Surface Lysines

In an aspect, the disclosure provides an ORF1 molecule comprising the amino acid sequence of an Anellovirus ORF1 protein (or an amino acid sequence having at least 75%, 80%, 85%, 90%, 95%, 96%, 97%, 98%, or 99% sequence identity thereto), wherein at least 30%, 40%, 50%, 60%, 70%, 75%, 80%, 85%, 90%, 95%, 96%, 97%, 98%, 99%, or 100% of the lysine residues in the amino acid sequence of the Anellovirus ORF1 protein has been mutated (e.g., substituted with another amino acid, e.g., threonine, alanine, serine, asparagine, or glutamine).

In some embodiments, all but one lysine residue of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all lysine residues of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all but one of the lysine residues of the Anellovirus ORF1 protein are mutated (e.g., substituted with another amino acid, e.g., serine or alanine). In certain embodiments, the one lysine residue not mutated is exposed on the surface of a proteinaceous exterior comprising the Anellovirus ORF1 protein. In some embodiments, all lysine residues of the Anellovirus ORF1 protein are mutated (e.g., substituted with another amino acid, e.g., serine or alanine). In some embodiments, the ORF1 molecule further comprises a lysine residue not found in the amino acid sequence of the Anellovirus ORF1 protein (e.g., a lysine residue inserted or substituted into the Anellovirus ORF1 protein sequence, or a lysine residue attached to the N-terminal or C-terminal end of the Anellovirus ORF1 protein sequence). Such ORF1 molecules may be useful, for example, for controlling covalent attachment of a surface moiety to a lysine residue in the proteinaceous exterior.

### Mutations of Surface Cysteines

In an aspect, the disclosure provides an ORF1 molecule comprising the amino acid sequence of an Anellovirus ORF1 protein (or an amino acid sequence having at least 75%, 80%, 85%, 90%, 95%, 96%, 97%, 98%, or 99% sequence identity thereto), wherein at least 30%, 40%, 50%, 60%, 70%, 75%, 80%, 85%, 90%, 95%, 96%, 97%, 98%, 99%, or 100% of the cysteine residues in the amino acid sequence of the Anellovirus ORF1 protein has been mutated (e.g., substituted with another amino acid, e.g., threonine, alanine, serine, asparagine, or glutamine).

In some embodiments, all but one cysteine residue of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all cysteine residues of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all but one of the cysteine residues of the Anellovirus ORF1 protein are mutated (e.g., substituted with another amino acid, e.g., serine or alanine). In certain embodiments, the one cysteine residue not mutated is exposed on the surface of a proteinaceous exterior comprising the Anellovirus ORF1 protein. In some embodiments, all cysteine residues of the Anellovirus ORF1 protein are mutated (e.g., substituted with another amino acid, e.g., serine or alanine). In some embodiments, the ORF1 molecule further comprises a cysteine residue not found in the amino acid sequence of the Anellovirus ORF1 protein (e.g., a cysteine residue inserted or substituted into the Anellovirus ORF1 protein sequence, or a cysteine residue attached to the N-terminal or C-terminal end of the Anellovirus ORF1 protein sequence). Such ORF1 molecules may be useful, for example, for controlling covalent attachment of a surface moiety to a cysteine residue in the proteinaceous exterior.

### Polypeptides

The surface moiety can, in some instances, comprise a polypeptide. In some embodiments, the polypeptide is a about 5-10, 10-15, 15-20, 20-25, 25-30, 30-40, 40-50, 50-100, 100-150, 150-200, 200-250, 250-300, 300-400, 400-500, 500-600, 600-700, 700-800, 800-900, 900-1000, 1000-1500, 1500-2000, 2000-2500, 2500-3000, 3000-4000, or 4000-5000 amino acids in length). In some embodiments, the surface moiety is a polypeptide fused to a protein of the anellovector or anelloVLP (e.g., an ORF1 molecule of the anellovector or anelloVLP). In certain embodiments, the peptide is linear or branched.

In some embodiments, the surface moiety comprises an antibody molecule (e.g., an antibody or an antigen-binding fragment thereof). In certain embodiments, the surface moiety comprises an Fv, Fab, Fab′, Fab′-SH, F(ab′)₂, diabody, linear antibody, single-chain antibody molecule (e.g. scFv), or a multispecific antibody formed from antibody fragments. In certain embodiments, the surface moiety is a multispecific antibody molecule (e.g., a bispecific antibody molecule or a trispecific antibody molecule). In some embodiments, the surface moiety is selected from a hormone, cytokine, enzyme, transcription factor, receptor, ligand, transporter, secreted protein, carrier protein, structural protein, or a functional fragment thereof (e.g., as described herein). In some embodiments, the surface moiety comprises a polypeptide effector (e.g., as described herein). In certain embodiments, the surface moiety comprises a therapeutic effector (e.g., as described herein). In embodiments, the surface moiety comprises a regulatory intracellular polypeptide (e.g., as described herein). In embodiments, the surface moiety comprises a secreted polypeptide effector (e.g., as described herein). In embodiments, the surface moiety comprises a viral polypeptide or peptide. In embodiments, the surface moiety comprises a SARS-CoV-2 polypeptide or peptide (e.g., a receptor binding domain, e.g., of a spike protein, e.g., as described herein), or a polypeptide having at least 75%, 80%, 85%, 90%, 95%, 96%, 97%, 98%, or 99% sequence identity thereto. In embodiments, the surface moiety comprises a polypeptide having at least 75%, 80%, 85%, 90%, 95%, 96%, 97%, 98%, 99%, or 100% amino acid sequence identity to amino acids 319-541 from SARS-CoV-2 coronavirus spike protein (e.g., according to ThermoFisher Catalog #RP-87704). In some embodiments, the surface moiety comprises a binding moiety (e.g., a biotin moiety). In some embodiments, the surface moiety comprises a fluorophore (e.g., an Alexa Fluor moiety, e.g., Alexa Fluor 647).

In some embodiments, a polypeptide surface moiety is displayed on the surface of the anellovector or anelloVLP. In some embodiments, the surface moiety is covalently attached to the surface (e.g., proteinaceous exterior) of the anellovector or anelloVLP. In certain embodiments, the surface moiety is a polypeptide fused to an ORF1 molecule. In embodiments, the surface moiety is a heterologous domain of an ORF1 molecule. In embodiments, the surface moiety replaces a region (e.g., a subdomain as described herein, e.g., an HVR) of an ORF1 protein. In embodiments, all copies of the ORF1 molecule in the proteinaceous exterior of an anellovector or anelloVLP are fused to copies of the surface moiety. In embodiments, some copies of the ORF1 molecule in the proteinaceous exterior of an anellovector or anelloVLP are fused to copies of the surface moiety and some copies of the ORF1 molecule in the proteinaceous exterior of an anellovector or anelloVLP are not fused to copies of the surface moiety. In embodiments, some copies of the ORF1 molecule in the proteinaceous exterior of an anellovector or anelloVLP are fused to copies of a first surface moiety and some copies of the ORF1 molecule in the proteinaceous exterior of an anellovector or anelloVLP are fused to copies of a second surface moiety.

In some embodiments, the surface moiety is noncovalently attached to the surface (e.g., proteinaceous exterior) of the anellovector or anelloVLP. For example, the surface moiety may comprise a binding domain that binds to a region on the surface (e.g., proteinaceous exterior) of the anellovector or anelloVLP. In an embodiment, the surface moiety comprises an antibody molecule that specifically binds to an ORF1 molecule of the proteinaceous exterior of the anellovector or anelloVLP.

### Genetic Grafting of ORF1 to a Surface Moiety

In some embodiments, an ORF1 molecule comprises the amino acid sequence of a surface moiety, e.g., as described herein. An ORF1 molecule may be fused (e.g., at the N-terminus or C-terminus) to the surface moiety. In some embodiments, a surface moiety is grafted into the sequence of the ORF1 molecule. For example, a surface moiety may be inserted within or between domains of the ORF1 molecule. In some instances, a surface moiety replaces at least a portion (e.g., at least 5-10, 10-20, 20-30, 30-40, 40-50, 50-60, 60-70, 70-80, 80-90, 90-100, 100-150, 150-200, or 200-300 contiguous amino acid residues) of a domain of the ORF1 molecule. In some embodiments, the surface moiety replaces the entirety of at least one domain of the ORF1 molecule.

In some embodiments, an ORF1 molecule grafted to a surface moiety comprises a deletion or truncation of a structural arginine-rich region. In certain embodiments, the surface moiety replaces the structural arginine-rich region, or a portion thereof (e.g., a portion consisting of 5-10, 10-15, 15-20, 20-30, 30-35, or 35-40 amino acids thereof). In certain embodiments, the surface moiety is grafted at or within 1-5, 5-10, 10-15, 15-20, 20-25, 25-30, 30-35, or 35-40 amino acid residues of a structural jelly-roll region or a jelly-roll B—H strands subdomain as described herein.

In some embodiments, an ORF1 molecule grafted to a surface moiety comprises a deletion or truncation of a structural hypervariable region (HVR) (e.g., as described herein). In certain embodiments, the surface moiety replaces the structural HVR, or a portion thereof (e.g., a portion consisting of 5-10, 10-15, 15-20, 20-30, 30-40, 40-50, 50-60, 60-70, 70-80, 80-90, 90-100, 100-110, 110-120, 120-130, 130-140, or 140-150 amino acids thereof). In some embodiments, an ORF1 molecule grafted to a surface moiety comprises a deletion or truncation of one or more of a P1-1 domain, P2 domain, and/or P1-2 domain (e.g., as described herein). In certain embodiments, the surface moiety replaces the P1-1 domain, P2 domain, and/or P1-2 domain, or a portion thereof (e.g., a portion consisting of 5-10, 10-15, 15-20, 20-30, 30-40, 40-50, 50-60, 60-70, 70-80, 80-90, 90-100, 100-110, 110-120, 120-130, 130-140, or 140-150 amino acids thereof). In certain embodiments, the surface moiety is grafted at or within 1-5, 5-10, 10-15, 15-20, 20-25, 25-30, 30-35, 35-40, 40-50, 50-60, 60-70, 70-80, 80-90, 90-100, 100-125, 125-150, 150-175, or 175-200 amino acid residues of the C-terminal end of a structural jelly-roll region or jelly-roll B—H strands subdomain as described herein. In certain embodiments, the surface moiety is grafted at or within 1-5, 5-10, 10-15, 15-20, 20-25, 25-30, 30-35, 35-40, 40-50, 50-60, 60-70, 70-80, 80-90, 90-100, 100-125, 125-150, 150-175, or 175-200 amino acid residues of the N-terminal end of a structural N22 domain, jelly-roll I strand subdomain, or structural CTD as described herein.

In some embodiments, an ORF1 molecule grafted to a surface moiety comprises a deletion or truncation of a structural C-terminal domain (CTD). In certain embodiments, the surface moiety replaces the structural CTD, or a portion thereof (e.g., a portion consisting of 5-10, 10-15, 15-20, 20-30, 30-40, 40-50, 50-60, 60-70, 70-80, 80-90, 90-100, 100-110, 110-120, or 120-130 amino acids thereof). In certain embodiments, the surface moiety is grafted at or within 1-5, 5-10, 10-15, 15-20, 20-25, 25-30, 30-35, 35-40, 40-50, 50-60, 60-70, 70-80, 80-90, 90-100, 100-125, 125-150, 150-175, or 175-200 amino acid residues of a structural N22 domain or a jelly-roll I strand subdomain as described herein.

### Exemplary ORF1 Molecules Attached to Surface Moieties

In some embodiments, a surface moiety and ORF1 fusion protein comprises an amino acid sequence as listed in Table E1 below, or an amino acid sequence having at least 75%, 80%, 85%, 90%, 95%, 96%, 97%, 98%, or 99% sequence identity thereto. In some embodiments, a surface moiety comprises an amino acid sequence as listed in Table E1 below, or an amino acid sequence having at least 75%, 80%, 85%, 90%, 95%, 96%, 97%, 98%, or 99% sequence identity thereto. In some embodiments, a surface moiety comprises a CS protein, or a functional fragment thereof (e.g., a fragment comprising the 184 C-terminal residues of a CS protein). In some embodiments, a surface moiety comprises a hepatitis B virus surface antigen. In some embodiments, a surface moiety comprises one or more (e.g., 1, 2, 3, 4, 5, or 6) NANP peptides (e.g., NANP-2 peptides). In some embodiments, a surface moiety comprises a cysteine residue (e.g., a C-terminal cysteine residue). In some embodiments, a surface moiety comprises an NHS click handle (e.g., as described herein).

In an aspect, the disclosure provides a nucleic acid molecule encoding a fusion protein comprising a surface moiety and an ORF1 molecule (e.g., as described herein).

In an aspect, the disclosure provides a polypeptide comprising a CCN5 protein and a C-terminal cysteine residue. In some embodiments, the polypeptide comprises the amino acid sequence of CCN5 CTerMCys as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising an aflibercept protein and a C-terminal cysteine residue. In some embodiments, the polypeptide comprises the amino acid sequence of aflibercept as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising ranibizumab and a C-terminal cysteine residue. In some embodiments, the polypeptide comprises the amino acid sequence of ranibizumab HC malE_CTermCys as listed in Table E1 below, or an amino acid sequence having at least 75%, 80%, 85%, 90%, 95%, 96%, 97%, 98%, or 99% sequence identity thereto. In some embodiments, the polypeptide comprises the amino acid sequence of scFab RaniHC-link50-LC malE_CTermCys as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising bevacizumab and a C-terminal cysteine residue. In some embodiments, the polypeptide comprises the amino acid sequence of bevacizumab HC malE_CTermCys as listed in Table E1 below, or an amino acid sequence having at least 75%, 80%, 85%, 90%, 95%, 96%, 97%, 98%, or 99% sequence identity thereto.

### X-Fold Symmetry

In some embodiments, an ORF1 molecule as described herein comprises a surface moiety situated at a position such that, when the ORF1 molecule is complexed with one or more additional copies of the ORF1 molecules (each attached to another surface moiety, e.g., copies of the same surface moiety or one or more different surface moieties), the surface moieties form a multimer. In certain embodiments, a complex of two such ORF1 molecules attached to surface moieties results in the formation of a dimer of the surface moieties. In certain embodiments, a complex of three such ORF1 molecules attached to surface moieties results in the formation of a trimer of the surface moieties. In certain embodiments, a complex of five such ORF1 molecules attached to surface moieties results in the formation of a pentamer of the surface moieties.

To form a multimer of surface moieties, the surface moieties are generally attached to the ORF1 molecule at a surface-exposed portion of the ORF1 molecule. In some embodiments, the surface-exposed portion of the ORF1 molecule is part of a structural hypervariable region (HVR) of the ORF1 molecule, e.g., as described herein. In some embodiments, the surface-exposed portion of the ORF1 molecule is part of a P1 domain (e.g., a P1-1 subdomain or a P1-2 subdomain) of the ORF1 molecule, e.g., as described herein. In some embodiments, the surface-exposed portion of the ORF1 molecule is part of a P2 domain of the ORF1 molecule, e.g., as described herein. In certain embodiments, the surface moiety is fused to, replaces, or is attached at or between residues of the ORF1 molecule corresponding to positions 284-285 of a Ring10 ORF1 protein. In certain embodiments, the surface moiety is fused to, replaces, or is attached at or between residues of the ORF1 molecule corresponding to positions 328-329 of a Ring10 ORF1 protein. In certain embodiments, the surface moiety is fused to, replaces, or is attached at or between residues of the ORF1 molecule corresponding to positions 256-383 of a Ring10 ORF1 protein. In certain embodiments, the surface moiety is fused to, replaces, or is attached at or between residues of the ORF1 molecule corresponding to positions 251-383 of a Ring10 ORF1 protein. In certain embodiments, the surface moiety is fused to, replaces, or is attached at or between residues of the ORF1 molecule corresponding to positions 251-384 of a Ring10 ORF1 protein. In certain embodiments, the surface moiety is attached at the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR or P2 domain).

In an aspect, the disclosure provides an ORF1 molecule comprising the amino acid sequence of an ORF1 protein comprising at least one mutation (e.g., deletion, substitution, addition, insertion, or frameshift) in a surface-exposed region relative to a wild-type ORF1 protein sequence (e.g., as described herein. In some embodiments, the surface-exposed region comprises the region corresponding amino acids 251-386 of the amino acid sequence of Ring 10 ORF1 (e.g., as described herein).

In an aspect, the disclosure provides an ORF1 molecule comprising the amino acid sequence of an ORF1 protein comprising at least one mutation (e.g., deletion, substitution, addition, insertion, or frameshift) in the HVR relative to a wild-type ORF1 protein sequence (e.g., as described herein).

In some embodiments, the glutamine residue corresponding to Q287 of Ring 10 (e.g., as described herein) has been mutated (e.g., substituted). In some embodiments, the glutamine residue corresponding to Q287 of Ring 10 has been mutated to a cysteine residue. In certain embodiments, the ORF1 molecule comprises the amino acid sequence of the Ring 10 ORF1 protein, or an amino acid sequence having at least 75%, 80%, 85%, 90%, 95%, 96%, 97%, 98%, or 99% sequence identity thereto, and a Q287C mutation.

In some embodiments, the threonine residue corresponding to T365 of Ring 10 (e.g., as described herein) has been mutated (e.g., substituted). In some embodiments, the threonine residue corresponding to T365 of Ring 10 has been mutated to a cysteine residue. In certain embodiments, the ORF1 molecule comprises the amino acid sequence of the Ring 10 ORF1 protein, or an amino acid sequence having at least 75%, 80%, 85%, 90%, 95%, 96%, 97%, 98%, or 99% sequence identity thereto, and a T365C mutation.

In an aspect, the disclosure provides a polypeptide comprising an amino acid sequence as listed in Table E1 below, or an amino acid sequence having at least 75%, 80%, 85%, 90%, 95%, 96%, 97%, 98%, or 99% sequence identity thereto. In some embodiments, the polypeptide further comprises the amino acid sequence of an ORF1 protein (e.g., as described herein), or an amino acid sequence having at least 75%, 80%, 85%, 90%, 95%, 96%, 97%, 98%, or 99% sequence identity thereto.

In an aspect, the disclosure provides a nucleic acid molecule encoding a polypeptide comprising an amino acid sequence as listed in Table E1 below, or an amino acid sequence having at least 75%, 80%, 85%, 90%, 95%, 96%, 97%, 98%, or 99% sequence identity thereto. In some embodiments, the polypeptide encoded by the nucleic acid molecule further comprises the amino acid sequence of an ORF1 protein (e.g., as described herein), or an amino acid sequence having at least 75%, 80%, 85%, 90%, 95%, 96%, 97%, 98%, or 99% sequence identity thereto. In some embodiments, an anellovector as described herein comprises a proteinaceous exterior comprising an ORF1 molecule as described above. In some embodiments, an anellovector as described herein comprises a proteinaceous exterior comprising an ORF1 molecule encoded by a nucleic acid molecule as described above. In some embodiments, an anelloVLP as described herein comprises a proteinaceous exterior comprising an ORF1 molecule as described above. In some embodiments, an anelloVLP as described herein comprises a proteinaceous exterior comprising an ORF1 molecule encoded by a nucleic acid molecule as described above.

### Nucleic Acid Molecules

The surface moiety may, in some instances, comprise a nucleic acid molecule. In some embodiments, the surface moiety comprises DNA (e.g., single-stranded DNA or double-stranded DNA). In some embodiments, the surface moiety comprises RNA (e.g., single-stranded RNA or double-stranded RNA). In some embodiments, the surface moiety comprises DNA and RNA (e.g., a strand of DNA hybridized to a strand of RNA). In some embodiments, the surface moiety comprises an oligonucleotide (e.g., an oligonucleotide having a length of about 5-10, 10-15, 15-20, 20-25, 25-30, 30-35, 35-40, 40-45, 45-50, 50-60, 60-70, 70-80, 80-90, or 90-100 nucleotides). In some embodiments, the surface moiety comprises a functional nucleic acid molecule (e.g., a functional RNA). In certain embodiments, the surface moiety comprises an mRNA, siRNA, miRNA, or tRNA.

### Small Molecules

The surface moiety may, in some instances, comprise a small molecule. In some embodiments, the small molecule has a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole. In some embodiments, the small molecule is a salt, ester, or other pharmaceutically acceptable form of such compounds. Small molecules may include, but are not limited to, a neurotransmitter, a hormone, a drug, a toxin, a viral or microbial particle, a synthetic molecule, and an agonist or an antagonist. In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumor suppressors). One or a combination of molecules from the categories and examples described herein or from Orme-Johnson 2007, Methods Cell Biol. 2007; 80:813-26 (incorporated by reference in its entirety) can be used. In one embodiment, the small molecule comprises an antibiotic, anti-inflammatory drug, angiogenic or vasoactive agent, growth factor or chemotherapeutic agent.

Examples of small molecules that can be used as surface moieties as described herein include, without limitation, those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

### Vaccines/Antigens

In some instances, a surface moiety comprises an antigen (e.g., an antigen recognized by the immune system of a subject to be delivered the anellovector or anelloVLP). In some embodiments, a surface moiety comprises a vaccine. In some embodiments, a surface moiety comprises an epitope from a bacterium, virus, fungus, or parasite. In some embodiments, a surface moiety comprises a vaccine for a pathogen (e.g., the surface moiety comprises an antigen of the pathogen). In certain embodiments, the surface moiety comprises one or more vaccine antigens as listed in Table U1 below. In embodiments, the surface moiety comprises one vaccine antigen listed in Table U1. In embodiments, the surface moiety comprises a plurality of distinct vaccine antigens listed in Table U1 (e.g., a plurality of distinct vaccine antigens listed in a single row of Table U1).

TABLE U1
Exemplary vaccine antigens that can be included in a surface
moiety of an anellovector or anello VLP as described herein.

Ascomycota, e.g., Eurotiomycetes, e.g., Eurotiales, e.g., Trichocomaceae, e.g., Aspergillus species antigen (e.g.,
Aspergillus versicolor AVS)
Monodnaviria, e.g., Shotokuvirae, e.g., Cossaviricota, e.g., Papovaviricetes, e.g., Sepolyvirales, e.g., Polyomaviridae,
e.g., Betapolyomavirus, e.g., BK Virus antigen
Proteobacteria, e.g., Betaproteobacteria, e.g., Burkholderiales, e.g., Alcaligenaceae, e.g., Bordetella, e.g., Bordetella
pertussis antigen (e.g., pertussis toxin, filamentous haemagglutinin, pertactin)
Spirochaetes, e.g., Spirochaetales, e.g., Spriochaetaceae, e.g., Borrelia, e.g., Borrelia burgdorferi antigen
Proteobacteria, e.g., Betaproteobacteria, e.g., Burkholderiales, e.g., Burkholderiaceae, e.g., Burkholderia, e.g.,
Burkholderia species antigen (e.g., hydratase/aldolase PhnE)
Riboviria, e.g., Orthornavirae, e.g., Kitrinoviricota, e.g. Alsuviricetes, e.g., Martellivirales, e.g., Togaviridae, e.g.,
Alphavirus, e.g., Chikungunya virus (CHIKV) antigens
Chlamydiae, e.g., Chlamydiales, e.g., Chlamydiaceae, e.g., Chlamydia, e.g., Chlamydia trachomatis antigen
Proteobacteria, e.g., Gammaproteobacteria, e.g., Vibrionales, e.g., Vibrionaceae, e.g., Vibrio, e.g., Vibrio cholerae, e.g.,
Cholera antigen (e.g., cholera toxin B subunit)
“Firmicutes”, e.g., Clostridia, e.g., Clostridiales, e.g., Peptostreptococcaceae, e.g., Clostridioides, e.g., Clostridium, e.g.,
Clostridium difficile antigen
Alveolata, e.g., Apicomplexa, e.g., Conoidasida, e.g., Eucoccidiorida, e.g., Eimeriorina, e.g., Cryoptosporidiidae, e.g.,
Cryptosporidium antigen
Duplodnaviria, e.g., Heunggongvirae, e.g., Peploviricota, e.g., Herviviricetes, e.g., Herpesvirales, e.g., Herpesviridae,
e.g., Betaherpesvirinae, e.g., Cytomegalovirus (CMV) antigen
“Actinobacteria”, e.g., Acintobacteria, e.g., Mycobacteriales, e.g., Corynebacteriaceae, e.g., Corynebacterium,
Diphtheria antigen (e.g., diphtheria toxoid)
Amoebozoa, e.g., Entamoebidae, e.g., Entamoeba, e.g., Entamoeba histolytica antigen
Riboviria, e.g., Orthornavirae, e.g., Pisuviricota, e.g., Pisoniviricetes, e.g., Picornavirales, e.g., Picornaviridae, e.g.,
Enterovirus, e.g., Enterovirus 71 antigen (e.g., VP1)
Duplodnaviria, e.g., Heunggongvirae, e.g., Peploviricota, e.g., Herviviricetes, e.g., Herpesviridae, e.g.,
Lymphocryptovirus, e.g., Epstein-Barr virus antigen
Proteobacteria, e.g., Gammaproteobacteria, e.g., Enterobacterlaes, e.g., Enterobacteriaceae, e.g., Escherichia, e.g.,
Escherichia coli antigen (e.g., bifunctional penicillin-binding protein)
Riboviria, e.g., Orthornavirae, e.g., Kitrinoviricota, e.g., Flasuviricetes, e.g., Amarillovirales, Flaviviridae genus
(Dengue Virus) antigen
Proteobacteria, e.g., Epsilonproteobacteria, e.g., Campylobacterales, e.g., Helicobacteraceae, e.g., Helicobacter, e.g.,
Helicobacter pylori antigen
Hepatitis antigen (e.g., hepatitis A (HAV), e.g., B (HBV), e.g., C (HCV), e.g., D (HDV) e.g., G (HGV) virus antigen)
Duplodnaviria, e.g., Heunggongvirae, e.g., Peploviricota, e.g., Herviviricetes, e.g., Herpesviridae, e.g.,
Alphaherpesvirinae, e.g., Simplexvirus, e.g., Herpes simplex antigen
Varidnaviria, e.g., Bamfordvirae, e.g., Preplasmiviricota, e.g., Tectiliviricetes, e.g., Rowavirales, e.g., Adenoviridae,
e.g., Aviadenovirus, e.g., Ichtadenovirus, e..g, Testadenovirus, e.g., Atadenovirus, e.g., Mastadenovirus, e.g.,
Siadenovirus Human adenovirus antigen (e.g., type 34 hexon protein)
Riboviria, e.g., Pararnavirae, e.g., Artverviricota, e.g., Revtraviricetes, e.g., Ortervirales, e.g., Retroviridae, e.g.,
Orthoretrovirinae, e.g., Lentivirus, e.g., Human immunodeficiency virus (HIV) antigen (e.g., GP-120, GP-160)
Riboviria, e.g., Orthornavirae, e.g., Negarnaviricota, e.g., Monjiviricetes, e.g., Mononegavirales, e.g., Pneumoviridae,
e.g., Metapneumovirus, e.g., Human metapneumovirus antigen
Monodnaviria, e.g., Shotokuvirae, e.g., Cossaviricota, e.g., Papovaviricetes, e.g., Zurhausenvirales, e.g.,
Papillomaviridae, e.g, Alphapapillomavirus, e.g., Betapapillomavirus, e.g., Chipapillomavirus, e.g., Deltapapillovirus,
e.g., Dyochipapillomavirus, e.g., Dyodeltapapillomavirus, e.g., Dyoepsilonpapillomavirus, e.g., Dyoetapapillomavirus,
e.g., Dyoiotapapillomavirus, e.g., Dyokappapapillomavirus, e.g., Dyolambdapapillomavirus, e.g.,
Dyomupapillomavirus, e.g., Dyonupapillomavirus, e.g., Dyoomegapapillomavirus, e.g., Dyoomikronpapillomavirus,
e.g., Dyophipapillomavirus, e.g., Dyopipapillomavirus, e.g., Dyopsipapillomavirus, e.g., Dyorhopapillomavirus, e.g.,
Dyosigmapapillomavirus, e.g., Dyotaupapillomavirus, e.g., Dyothetapapillomavirus, e.g., Dyoupsilonpapillomavirus,
e.g., Dyoxipapillomavirus, e.g., Dyozetapapillomavirus e.g., Gammapapillomavirus, e.g., Iotapapillomavirus, e.g.,
Kappapapillomavirus, e.g., Lambdapapillomavirus, e.g., Mupapillomavirus, e.g., Nupapillomavirus, e.g.,
Omegapapillomavirus, e.., Omikronpapillomavirus, e.g., Phipapillomavirus, e.g., Pipapillomavirus, e.g.,
Psipapillomavirus, e.g., Rhopapillomavirus, e.g., Sigmapapillomavirus, e.g., Taupapillomavirus, e.g.,
Thetapapillomavirus, e.g., Treisdeltapapillomavirus, e.g., Treisepsiolonpapillomavirus, e.g., Treisetapapillomavirus,
e.g., Treisiotapapillomavirus, e.g., Trieskappapapillomavirus, e.g., Treisthetapapillomavirus, e.g.,
Treiszetapapillomavirus, e.g., Upsilonpapillomavirus, e.g., Xipapillomavirus, e.g., Zetapapillomavirus, e.g.,
Alefpapillomavirus, e.g., Human papilloma virus (HPV) antigen (e.g., L1 major capsid protein)
Riboviria, e.g., Orthornavirae, e.g., Negarnaviricota, e.g., Insthoviricetes, e.g., Articulavirales, e.g., Orthomyxoviridae,
e.g., Alphainfluenzavirus, e.g., Betainfluenzavirus, e.g., Gammainfluenzavirus, e.g., Deltainfluenzavirus, e.g., Influenza
virus antigen (e.g., HA)
Monodnaviria, e.g., Shotokuvirae, e.g., Cossaviricota, e.g., Papovaviricetes, e.g., Sepolyvirales, e.g., Polyomaviridae,
e.g., Betapolyomavirus, e.g., JC Virus/JC polyomavirus antigen
Proteobacteria, e.g., gammaproteobacteria, e.g., Enterobacterales, e.g., Klebsiella, e.g., Klebsiella pneumoniae antigen
Riboviria, e.g., Orthornavirae, e.g., Negarnaviricota, e.g., Ellioviricetes, e.g., Bunyavirales, e.g., Arenaviridae, e.g.,
Mammarenavirus, e.g., Lassa virus antigen
Euglenozoa, e.g., Kinetoplastea, e.g., Trypanosomatida, e.g., Leishmania promastigotes antigen
Spriochaetes, e.g., Spirochaetales, e.g., Leptospiraceae, e.g., Leptospira, e.g., Leptospira interrogans antigen
Proteobacteria, e.g., Betaproteobacteria, e.g., Neisseriales, e.g., Neisseriaceae, e.g., Neisseria, e.g., Meningococcal
antigen (e.g., capsular polysaccharides of serogroup A, B, C, Y and W-135)
Actinobacteria, e.g., Corynebacteriales, e.g., Mycobacteriaceae, e.g., Mycobacterium, e.g., Mycobacterium tuberculosis
antigen
Proteobacteria, e.g., etaproteobacteria, e.g., Neisseriales, e.g., Neisseriaceae, e.g., Neisseria, e.g., N.
meningitidis antigen (e.g., type Y polysaccharide [MenY], type W polysaccharide [MenW])
Proteobacteria, e.g., etaproteobacteria, e.g., Neisseriales, e.g., Neisseriaceae, e.g., Neisseria, e.g., Neisseriea gonorrhea
antigen
Apicomplexa, e.g., Aconoidasida, e.g., Haemospororida, e.g., Plasmodiidae, e.g., Plasmodium, e.g., Plasmodium
species antigen (e.g., HRPII, pLDH, pAldo)
“Firmicutes”, e.g., Bacilli, e.g., Lactobacillusoccaceae, e.g., Streptococcus, e.g., Pneumococcal antigen
(e.g., Streptococcus pneumoniae type 3 capsular polysaccharide)
Proteobacteria, e.g., Gammaproteobacteria, e.g., Pseudomonadales, e.g., Pseudomonadaceae, e.g., Pseudomonas, e.g.,
Pseudomonas species antigen (e.g., ferredoxin reductase component)
Riboviria, e.g., Orthornavirae, e.g., Negarnaviricota, e.g., Monjiviricetes, e.g., Mononegavirales, e.g., Rhabdoviridae,
e.g., Lyssavirus, e.g., Rabies antigen
Riboviria, e.g., Orthornavirae, e.g., Negarnaviricota, e.g., Monjiviricetes, e.g., Mononegavirales, e.g., Pneumoviridae,
e.g., Orthopneumovirus, e.g., Respiratory syncytial virus (RSV) antigen
Riboviria, e.g., Orthornavirae, e.g., Negarnaviricota, e.g., Ellioviricetes, e.g., Bunyavirales, e.g., Phenuviridae, e.g.,
Phlebovirus, e.g., Rift Valley fever Virus antigen
Riboviria, e.g., Orthornavirae, e.g., Duplornaviricota, e.g., Resentoviricetes, e.g., Reovirales, e.g., Reoviridae, e.g.,
Sedoreovirinae, e.g., Rotavirus, e.g., Rotavirus antigen (e.g., VP8, H1N1 M2, H7N9 F)
Proteobacteria, e.g., Gammaproteobacteria, e.g., Enterobacterales, e.g., Enterobacteriaceae, e.., Salmonella, e.g.,
Salmonella typhi antigen (e.g., Vi polysaccharide)
Platyhelminthes, e.g., Diplostomida, e.g., Schistosomatidae, e.g., Schistosomatinae, e.g., Schistosoma, e.g.,
Schistosoma species antigen
Proteobacteria, e.g., Gammaproteobacteria, e.g., Enterobacterales, e.g., Enterobacteriaceae, e.g., Shigell, e.g., Shigella
antigen (e.g., shigella polysaccharides)
“Firmicutes”, e.g., Bacilli, e.g., Bacillales, e.g., Staphylococcaceae, e.g., Staphylococcus, e.g., Staphylococcal antigen
(e.g., staphylococcal enterotoxin B)
“Firmicutes”, e.g., Bacilli, e.g., Lactobacillales, e.g., Streptococcaceae, e.g., Streptococcus, e.g., Streptococcus
agalactiae antigen
“Firmicutes”, e.g., Bacilli, e.g., Lactobacillales, e.g., Streptococcaceae, e.g., Streptococcus, e.g., Streptococcus
pyogenes antigen
“Firmicutes”, e.g., Clostridia, e.g., Clostridiales, e.g., Clostridiaceae, e.g., Clostridium, e.g., Tetanus antigen (e.g.,
tetanus toxoid)
Riboviria, e.g., Orthornavirae, e.g., Kitrinoviricota, e.g., Flasuviricetes, e.g., Amarillovirales, e.g., Flaviviridae, e.g.,
Flavivirus, e.g., Tick-borne encephalitis (TBE) antigen
Apicomplexa, e.g., Conoidasida, e.g., Eucoccidiorida, e.g., Sarcocystidae, e.g., Toxoplasmatinae, e.g., Toxoplasma,
e.g., Toxoplasma gondii antigen
Spirochaetes, e.g., Spirochaetales, e.g., Spirochaetaceae, e.g., Treponema, e.g., Treponema pallidum antigen
“Actinobacteria”, e.g., Actinobacteria, e.g., Mycobacteriales, e.g., Mycobacteriaceae, e.g., Mycobacterium, e.g.,
Tuberculosis antigen
Riboviria, e.g., Orthornavirae, e.g., Negarnaviricota, e.g., Monjiviricetes, e.g., Mononegavirales, e.g., Rhabdoviridae,
e.g., Vesiculovirus, e.g., Vesicular stomatitis virus antigen (e.g., vesicular stomatitis virus glycoprotein)
Proteobacteria, e.g., Gammaproteobacteria, e.g., Vibrionales, e.g., Vibrionaceae, e.g., Vibrio, e.g., Vibrio cholerae
antigen
Riboviria, e.g., Orthornavirae, e.g., Kitrinoviricota, e.g., Flasuviricetes, e.g., Amarillovirales, e.g., Flaviviridae, e.g.,
Flavivirus, e.g., West Nile virus (WNV) antigen
Riboviria, e.g., Orthornavirae, e.g., Kitrinoviricota, e.g., Flasuviricetes, e.g., Amarillovirales, e.g., Flaviviridae, e.g.,
Flavivirus, e.g., Zika virus antigen
Apicomplexa, e.g., Aconoidasida, e.g., Haemospororida, e.g., Plasmodiidae, e.g., Plasmodium, e.g., Plasmodium vivax,
Plasmodium falciparum, Plasmodium malariae, Plasmodium ovale, or Plasmodium knowlesi.
Riboviria, e.g., Orthornavirae, e.g., Pisuviricota, e.g., Pisoniviricetes, e.g., Nidovirales, e.g., Coronaviridae, e.g.,
Betacoronavirus, e.g., Sebacovirus, e.g., severe acute respiratory syndrome-related coronavirus, e.g., SARS-CoV-2,
e.g., SARS-CoV-2 alpha variant, SARS-CoV-2 beta variant, SARS-CoV-2 gamma variant, SARS-CoV-2 delta variant,
or SARS-CoV-2 omicron variant

In some embodiments, a vaccine comprising an anellovector or anelloVLP as described herein is administered with an adjuvant. In certain embodiments, the adjuvant is an inorganic adjuvant (e.g., potassium alum, aluminium hydroxide, aluminium phosphate, or calcium phosphate hydroxide). In certain embodiments, the adjuvant is an oil-based adjuvant (e.g., paraffin oil). In certain embodiments, the adjuvant is a saponin. In certain embodiments, the adjuvant is a cytokine. In certain embodiments, the adjuvant is a squalene. In certain embodiments, the adjuvant is Freund's complete adjuvant.

In some embodiments, a vaccine as described herein is administered in a dose comprising about 10¹⁰ to 10¹⁴ viral genome equivalents of an anellovector as described herein. In some embodiments, a vaccine as described herein is administered as a dose comprising about 10¹⁰ to 10¹⁴ particles (e.g., anellovectors or anelloVLPs) as described herein.

### Ligands

In some instances, a surface moiety as described herein comprises a ligand (e.g., a ligand that binds specifically to a receptor on a target cell). In some embodiments, the ligand is a growth factor. In certain embodiments, the ligand binds to a growth factor receptor on the surface of the target cell. In some embodiments, the ligand is a cytokine. In some embodiments, the ligand is a hormone.

### II. Compositions and Methods for Making Anellovectors and AnelloVLPs

The present disclosure provides, in some aspects, anellovectors, anelloVLPs, and methods thereof for delivering effectors. In some embodiments, the anellovectors, anelloVLPs, or components thereof can be made as described below. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell. In some embodiments, the anellovectors, anelloVLPs, or components thereof can be made using a tandem construct, e.g., as described in PCT Publication No. WO 2021252955, which is incorporated herein by reference in its entirety. The present disclosure provides, in some aspects, compositions (e.g., bacmids, donor vectors, baculovirus particles, and cells comprising same) and methods that can be used for producing anellovectors or anelloVLPs, e.g., as described herein. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule). In some embodiments, the anellovectors, anelloVLPs, or components thereof can be made using a bacmid/insect cell system, e.g., as described as described in PCT Publication No. WO 2021/252943, which is incorporated herein by reference in its entirety. Methods of producing an anellovector using a host cell are described, for example, below in the section entitled “Host Cells.” Method of producing an anellovector in a cell-free system are described, for example, below in the section entitled “In vitro assembly methods”.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an Anelloviridae family vector (e.g., anellovector).

### Components and Assembly of Anellovectors and AnelloVLPs

The compositions and methods herein can be used to produce anellovectors and anelloVLPs. As described herein, an anellovector generally comprises a genetic element (e.g., a single-stranded, circular DNA molecule, e.g., comprising a 5′ UTR region as described herein) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As described herein, an anelloVLP generally comprises a proteinaceous exterior (e.g., comprising a polypeptide encoded by an Anellovirus ORF1 nucleic acid, e.g., as described herein) and an exogenous effector. As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described herein or as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a structural jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, an anellovector is assembled by enclosing a genetic element (e.g., as described herein) within a proteinaceous exterior in vitro (e.g., as described herein) (e.g., wherein the enclosing occurs external to a host cell, e.g., in the absence of a host cell). In some embodiments, the genetic element is enclosed within the proteinaceous exterior in a host cell (e.g., as described herein). In some embodiments, a surface moiety is attached to the proteinaceous exterior (e.g., as described herein). In some embodiments, the proteinaceous exterior comprises an ORF1 molecule comprising a surface moiety (e.g., as described herein). In certain embodiments, the ORF1 molecule comprises a surface moiety fused to an ORF1 domain.

In some embodiments, an anelloVLP is assembled by enclosing an exogenous effector within a proteinaceous exterior (e.g., as described herein) in vitro (e.g., wherein the enclosing occurs external to a host cell, e.g., in the absence of a host cell). In some embodiments, an anelloVLP is assembled by contacting a plurality of ORF1 molecules (e.g., as described herein) with an effector in vitro (e.g., wherein the contacting occurs external to a host cell, e.g., in the absence of a host cell). In some embodiments, an anelloVLP is assembled by attaching an exogenous effector to the exterior surface of a proteinaceous exterior (e.g., as described herein) in vitro. In some embodiments, a surface moiety is attached to the proteinaceous exterior (e.g., as described herein). In some embodiments, the proteinaceous exterior comprises an ORF1 molecule comprising a surface moiety (e.g., as described herein). In certain embodiments, the ORF1 molecule comprises a surface moiety fused to an ORF1 domain.

In some embodiments, a host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, a host cell produces a genetic element from a nucleic acid construct comprising the sequence of the genetic element. In some embodiments, the nucleic acid construct is selected from a plasmid, in vitro circularized nucleic acid molecule, viral nucleic acid molecule, minicircle, bacmid, or artificial chromosome. In some embodiments, the genetic element is excised from the nucleic acid construct and, optionally, converted from a double-stranded form to a single-stranded form (e.g., by denaturation). In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein. In other embodiments, the nucleic acid construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

In some embodiments, the host cell comprises a genetic element construct (e.g., a bacmid, plasmid, or minicircle). In some embodiments, the host cell comprises a bacmid comprising one or more sequences encoding Anellovirus ORF molecules (e.g., ORF1, ORF2, ORF2/2, ORF2/3, ORF1/1, and/or ORF1/2 ORF molecules), or functional fragments thereof. In some embodiments, proteinaceous exterior proteins are expressed from the bacmid. In embodiments, the proteinaceous exterior proteins expressed from the bacmid enclose a genetic element, thereby forming an anellovector. In some embodiments, the bacmid comprises a backbone suitable for replication of the nucleic acid construct in insect cells (e.g., Sf9 cells), e.g., a baculovirus backbone region. In some embodiments, the bacmid comprises a backbone region suitable for replication of the genetic element construct in bacterial cells (e.g., E. coli cells, e.g., DH 10Bac cells). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells). In some embodiments, the bacmid is introduced into the host cell via a baculovirus particle. In embodiments, the bacmid is produced by a producer cell, e.g., an insect cell (e.g., an Sf9 cell) or a bacterial cell (e.g., an E. coli cell, e.g., a DH 10Bac cell). In embodiments, the producer cell comprises a bacmid and/or a donor vector, e.g., as described herein. In embodiments, the producer cell further comprises sufficient cellular machinery for replication of the bacmid and/or donor vector.

### ORF1 Molecules, e.g., for Assembly of Anellovectors

An anellovector or anelloVLP as described herein generally comprises a proteinaceous exterior comprising a polypeptide encoded by an Anellovirus ORF1 nucleic acid (e.g., an Anellovirus ORF1 molecule or a splice variant or functional fragment thereof, e.g., as described herein). An ORF1 molecule may, in some embodiments, comprise one or more of: a first region comprising a structural arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising structural jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 structural arginine-rich region, structural jelly-roll region, structural N22 domain, hypervariable region, and/or structural C-terminal domain. In some embodiments, the proteinaceous exterior comprises an Anellovirus ORF1 structural jelly-roll region (e.g., as described herein). In some embodiments, the proteinaceous exterior comprises an Anellovirus ORF1 structural arginine-rich region (e.g., as described herein). In some embodiments, the proteinaceous exterior comprises an Anellovirus ORF1 structural N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 structural C-terminal domain (e.g., as described herein).

In some embodiments, the anellovector comprises an ORF1 molecule and/or a nucleic acid encoding an ORF1 molecule. In some embodiments, the anelloVLP comprises an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to a Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein, e.g., produced using a composition or construct as described herein) and/or an effector (e.g., an exogenous effector). In some embodiments, a plurality of ORF1 molecules may form a multimer, e.g., to produce a proteinaceous exterior. In some embodiments, the multimer may be a homomultimer. In other embodiments, the multimer may be a heteromultimer.

In some embodiments, a first plurality of anellovectors or anelloVLPs comprising an ORF1 molecule as described herein is administered to a subject. In some embodiments, a second plurality of anellovectors or anelloVLPs comprising an ORF1 molecule described herein, is subsequently administered to the subject following administration of the first plurality. In some embodiments the second plurality of anellovectors or anelloVLPs comprises an ORF1 molecule having the same amino acid sequence as the ORF1 molecule comprised by the anellovectors or anelloVLPs of the first plurality. In some embodiments the second plurality of anellovectors or anelloVLPs comprises an ORF1 molecule having at least 70%, 75%, 80%, 85%, 90%, 95%, 96%, 97%, 98%, 99%, or 100% amino acid sequence identity to the ORF1 molecule comprised by the anellovectors or anelloVLPs of the first plurality.

### ORF2 Molecules, e.g., for Assembly of Anellovectors

Producing an anellovector or anelloVLP using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector or anelloVLP does not comprise an ORF2 molecule, or a splice variant or functional fragment thereof, and/or a nucleic acid encoding an ORF2 molecule, or a splice variant or functional fragment thereof. In some embodiments, producing the anellovector or anelloVLP comprises expression of an ORF2 molecule, or a splice variant or functional fragment thereof, but the ORF2 molecule is not incorporated into the anellovector or anelloVLP. In some embodiments, producing the anellovector or anelloVLP comprises expression of an ORF2 molecule, or a splice variant or functional fragment thereof, but the ORF2 molecule is expressed from a nucleic acid other than Anelloviridae family vector.

### Production of Protein Components

Protein components of an anellovector or anelloVLP, e.g., ORF1, can be produced in a variety of ways, e.g., as described herein. In some embodiments, one or more protein components of an anellovector or anelloVLP, including, e.g., the proteinaceous exterior, are produced in a host cell (e.g., the same host cell that packages the genetic elements into the proteinaceous exteriors, thereby producing the anellovectors). In some embodiments, one or more protein components of an anellovector or anelloVLP, including, e.g., the proteinaceous exterior, are produced in a cell that does not comprise a genetic element and/or a genetic element construct (e.g., as described herein). In some embodiments, one or more protein components of an anellovector or anelloVLP are produced and then secreted from a host cell. In some embodiments, one or more protein components of an anellovector or anelloVLP are produced and then isolated from a host cell (e.g., by lysing the host cell).

### Baculovirus Expression Systems

A viral expression system, e.g., a baculovirus expression system, may be used to express proteins (e.g., for production of anellovectors or anelloVLPs), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF molecule, e.g., ORF1, ORF2, ORF2/2, ORF2/3, ORF1/1, or ORF1/2, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an att site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculovirual vector comprises one or more non-baculoviral promoters, e.g., a mammalian promoter or an Anellovirus promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoter suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include ie1, ie2, ie0, etI, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH10BAC® competent cells, containing the bacmid DNA with a mini-attTn7 target site. In some embodiments, the baculovirus expression vector, e.g., pFASTBAC® vector, may have a promoter, e.g., a dual promoter (e.g., polyhedrin promoter, p10 promoter). Commercially available pFASTBAC® donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector. In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria, e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

Exemplary baculoviral vectors, as well as compositions and methods relating thereto (e.g., for producing proteins from Anelloviridae family viruses) are described in PCT Publication No. WO/2021/252943, which is incorporated herein by reference in its entirety.

### Insect Cell Systems

The proteins described herein may be expressed in host cells (e.g., insect cells) infected or transfected with recombinant baculovirus or bacmid DNA, e.g., as described above. In some embodiments, the host or host cell is an insect cell (e.g., an Sf9 cell, Sf21 cell, or Hi5 cell). In some embodiments, the insect cell is derived from Bombyx mori, Mamestra brassicae, Spodopterafrugiperda, Trichoplusia ni, or Drosophila melanogaster. In some embodiments, the insect cell is selected from Sf9 and Sf21 cells derived from Spodopterafrugiperda and Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodopterafrugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolayer at a temperature between 25° to 30° C. with aeration. Additional information on insect cells is discussed, for example, in U.S. patent application Ser. Nos. 14/564,512 and 14/775,154, each of which is hereby incorporated by reference.

Exemplary insect cells, as well as compositions and uses relating to same (e.g., for producing proteins from Anelloviridae family viruses), are described in PCT Publication No. WO/2021/252943, which is incorporated herein by reference in its entirety.

### Mammalian Cell Systems

In some embodiments, the proteins described herein may be expressed in vitro in animal cell lines infected or transfected with a vector encoding the protein, e.g., as described herein. Animal cell lines envisaged in the context of the present disclosure include porcine cell lines, e.g., immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines suitable for expressing the proteins described herein include, but are not limited to cell lines of human or primate origin, such as human or primate kidney carcinoma cell lines.

### Recombinase-Based Production of Genetic Elements and Anelloviridae Family Vectors

A genetic element for an Anelloviridae family vector (e.g., an anellovector) may be produced via site-specific recombination of a genetic element construct to produce a circular nucleic acid molecule comprising the genetic element sequence. In some embodiments, the circular nucleic acid molecule is a double-stranded DNA minicircle. In some embodiments, the circular nucleic acid molecule is in turn converted to a circular single-stranded DNA molecule, which can in turn serve as the genetic element of an Anelloviridae family vector (e.g., an anellovector) as described herein. Generally, the genetic element construct comprises a set of recombinase recognition sequences flanking the sequence of a genetic element of an Anelloviridae family vector. The recombinase recognition sites may be recognized by a site-specific recombinase. The remainder of the genetic element construct may, in some instances, comprise a vector backbone comprising elements for replication of the construct in a cell, such as a mammalian cell or a bacterial cell.

Contacting the genetic element construct with the site-specific recombinase (e.g., in a host cell) may result in excision of the genetic element sequence from the remainder of the construct and the formation of two circular nucleic acid molecules, one comprising the genetic element sequence and the other comprising the remainder of the vector backbone. In some embodiments, the circular nucleic acid molecule (e.g., a minicircle) comprising the genetic element sequence is converted to cssDNA in the host cell (e.g., a mammalian host cell), and is then encapsulated in a proteinaceous exterior comprising ORF1 molecules or VP1 molecules (e.g., as described herein) to produce an Anelloviridae family vector. In other embodiments, the circular nucleic acid molecule (e.g., a minicircle) comprising the genetic element sequence is isolated from the host cell (e.g., a bacterial host cell). The isolated minicircles are then converted to cssDNA, and can then be encapsulated (e.g., in an in vitro setting, such as a cell-free solution) within a proteinaceous exterior comprising ORF1 molecules or VP1 molecules (e.g., as described herein) to produce an Anelloviridae family vector.

### Three-Plasmid Systems

In some embodiments, a site specific recombinase-based system for producing genetic elements comprises three plasmids: (1) a first plasmid (e.g., a vector plasmid) comprising the sequence of the genetic element, flanked by a pair of recombinase recognition sites; (2) an expression plasmid comprising a cassette encoding a site-specific recombinase (e.g., as described herein) capable of recognizing the recombinase recognition sites; and (3) a plasmid (e.g., a self-replicating rescue (SRR) plasmid) providing one or more Anelloviridae family viral proteins (e.g., Anellovirus ORF1, ORF3, and/or ORF3 molecules; or CAV VP1, VP2, and/or VP3 molecules), including a capsid protein (e.g., an ORF1 molecule or VP1 molecule, e.g., as described herein). A host cell may comprise all three plasmids. Expression of the site-specific recombinase from the expression plasmid enables recombination of the first plasmid to produce minicircles comprising the genetic element sequence (which may, in some instances, be converted to cssDNA in the cell). Capsid proteins (e.g., ORF1 molecules or VP1 molecules) can be expressed from the SRR plasmid, and can then form proteinaceous exteriors (e.g., as described herein) encapsulating the genetic elements (e.g., after conversion to cssDNA), thereby producing Anelloviridae family vector particles. In some embodiments, expression of an ORF2 or ORF3 molecule, or a VP2 or VP3 molecule, from the SRR plasmid contributes to replication and/or packaging. In some embodiments, the ORF1 molecule encoded by one of the plasmids comprises a deletion relative to a wild-type Anellovirus ORF1 protein (e.g., a deletion of all or part of an Anellovirus ORF1 structural arginine-rich region and/or structural C-terminal domain (CTD), e.g., as described herein).

In some embodiments, the portion of the first plasmid flanked by recombination recognition sites comprises, in order: (i) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a nucleic acid sequence encoding a transgene of interest; and (iii) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto.

In some embodiments, the portion of the first plasmid flanked by recombination recognition sites comprises, in order: (i) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; and (iii) a nucleic acid sequence encoding a transgene of interest.

Non-limiting examples of plasmids suitable for use in a three-plasmid recombinase system as described herein are provided in Tables Z1-Z10 below. Tables Z1-Z5 provide exemplary first plasmid and expression plasmid sequences for use in a three-plasmid system utilizing Cre recombinase to recombine a vector plasmid comprising loxP sites flanking the genetic element sequence. In some embodiments, the SRR plasmid comprises the sequence of an exemplary plasmid as described in the SRR constructs section below. Tables Z6-Z10 provide exemplary first plasmid and expression plasmid sequences for use in a three-plasmid system utilizing Bxb1 recombinase to recombine a vector plasmid comprising attB and attP sites flanking the genetic element sequence.

In some embodiments, a vector plasmid (e.g., suitable for use in a three-plasmid system as described herein) comprises the nucleic acid sequence of a vector plasmid as listed in any of Tables Z1 or Z2 (or an element or subsequence thereof as described in the respective table), or a nucleic acid sequence having at least 75%, 80%, 85%, 90%, 95%, 96%, 97%, 98%, or 99% identity thereto. In some embodiments, a recombined vector plasmid (e.g., a minicircle as described herein) comprises the nucleic acid sequence of a vector plasmid as listed in any of Tables Z3 or Z4 (or an element or subsequence thereof as described in the respective table), or a nucleic acid sequence having at least 75%, 80%, 85%, 90%, 95%, 96%, 97%, 98%, or 99% identity thereto. In some embodiments, a recombinase expression plasmid (e.g., suitable for use in a three-plasmid system as described herein) comprises the nucleic acid sequence of a vector plasmid as listed in Table Z5 (or an element or subsequence thereof as described in the table), or a nucleic acid sequence having at least 75%, 80%, 85%, 90%, 95%, 96%, 97%, 98%, or 99% identity thereto.

In some embodiments, a vector plasmid (e.g., suitable for use in a three-plasmid system as described herein) comprises the nucleic acid sequence of a vector plasmid as listed in any of Tables Z6 or Z7 (or an element or subsequence thereof as described in the respective table), or a nucleic acid sequence having at least 75%, 80%, 85%, 90%, 95%, 96%, 97%, 98%, or 99% identity thereto. In some embodiments, a recombined vector plasmid (e.g., a minicircle as described herein) comprises the nucleic acid sequence of a vector plasmid as listed in any of Tables Z8 or Z9 (or an element or subsequence thereof as described in the respective table), or a nucleic acid sequence having at least 75%, 80%, 85%, 90%, 95%, 96%, 97%, 98%, or 99% identity thereto. In some embodiments, a recombinase expression plasmid (e.g., suitable for use in a three-plasmid system as described herein) comprises the nucleic acid sequence of a vector plasmid as listed in Table Z10 (or an element or subsequence thereof as described in the table), or a nucleic acid sequence having at least 75%, 80%, 85%, 90%, 95%, 96%, 97%, 98%, or 99% identity thereto.

In any of the embodiments, the coding sequence of the transgene (e.g., eGFP) in the vector plasmids may be replaced with any transgene, e.g. a transgene encoding an exogenous effector, e.g., a therapeutic effector (e.g., as described herein).

In some embodiments, the SRR plasmid comprises the sequence of an exemplary plasmid as described in the SRR constructs section below.

TABLE Z1
Exemplary floxed Ring19 vector plasmid
for three-plasmid systems (pRTx-2847)

| | |
|---|---|
| Name | pRTx-2847 |
| Type | Plasmid |
| Description | pLox- |
| | Ring19ΔORF::fCMV_EGFP_WPRE_bGH- |
| | pA-Rand100. |
| Length | 5452 bp |

(SEQ ID NO: 2618)

| | |
|---|---|
| Annotations: | |
| Region/Element | Base range |
| Lox71 (loxP site) | 432-465 |
| GC-rich region | 529-640 |
| Initiator element | 813-828 |
| 5′ UTR conserved | 880-950 |
| domain | |
| Intron 1 | 892-979 |
| Full CMV promoter | 1004-1573 |
| Inert 5′ UTR | 1585-1619 |
| eGFP coding sequence | 1620-2339 |
| WPRE | 2343-2934 |
| bGHpA terminator | 2939-3166 |
| sequence | |
| 100 bp random stuffer | 3167-3266 |
| sequence | |
| Lox66 (loxP site) | 3271-3304 |
| LacI repressor binding | 3384-3406 |
| site (complement) | |
| Lac operon operator | 3386-3402 |
| (lacO) | |
| Lac operon promoter | 3410-3440 |
| (complement) | |
| C-tag | 3418-3429 |
| E coli catabolite activator | 3455-3476 |
| protein binding site | |
| (complement) | |
| Origin of replication | 3705-4378 |
| (pUC origin) | |
| (complement) | |
| Kanamycin resistance | 4530-5339 |
| (KanR) CDS | |
| (complement) | |

TABLE Z2
Exemplary floxed Ring19 vector plasmid after recombination
event (pRTx-2847, post-recombination)

| | |
|---|---|
| Name | pRTx-2847_post_recombine |
| Type | Circular DNA |
| Description | pLox- |
| | Ring19ΔORF::fCMV_EGFP_WPRE_bGH- |
| | pA-Rand100 after recombination by Cre |
| | recombinase |
| Length | 2839 bp |

(SEQ ID NO: 2619)

| | |
|---|---|
| Annotations: | |
| Region/Element | Base range |
| Full CMV promoter | 1-570 |
| Inert 5′ UTR | 582-616 |
| eGFP coding sequence | 617-1336 |
| WPRE | 1340-1931 |
| bHGpA terminator | 1936-2163 |
| sequence | |
| 100 bp random stuffer | 2164-2263 |
| sequence | |
| loxP site | 2268-2301 |
| GC-rich region | 2365-2476 |
| Initiator element | 2649-2664 |
| 5′ UTR conserved | 2716-2786 |
| domain | |
| Intron 1 | 2728-2815 |

TABLE Z3
Exemplary floxed Ring2 vector plasmid
for three-plasmid systems (pRTx-2846)

| | |
|---|---|
| Name | pRTx-2846 |
| Type | Plasmid |
| Description | pLox- |
| | Ring2ΔORF::fCMV_EGFP_WPRE_bGH- |
| | pA-Rand200 |
| Length | 5980 bp |

(SEQ ID NO: 2620)

| | |
|---|---|
| Annotations: | |
| Region/Element | Base range |
| Kanamycin resistance | 1031-1840 |
| (KanR) CDS (complement) | |
| T7 RNA polymerase | 2345-2362 |
| promoter | |
| Lox71 (loxP site) | 2412-2445 |
| VL46-002-LY2 gene | 2446-2610 |
| GC-rich region | 2501-2612 |
| VL46-002-LY2 gene | 2615-3035 |
| Promoter | 2634-2877 |
| Initiator element | 2869-2884 |
| 5′ UTR conserved domain | 2935-3005 |
| Origin of replication | 2935-3005 |
| Intron 1 | 2947-3035 |
| Full CMV promoter | 3060-3629 |
| Inert 5′UTR | 3641-3675 |
| eGFP CDS | 3676-4395 |
| WPRE | 4399-4990 |
| bGHpA terminator | 4995-5222 |
| sequence | |
| 200 bp random stuffer | 5223-5422 |
| sequence | |
| Lox66 (loxP site) | 5427-5460 |
| LacUV5 promoter | 5895-5925 |
| (complement) | |
| C-tag | 5903-5914 |
| CAP binding site | 5940-5961 |
| CAP binding site | 5940-5961 |
| (complement) | |

TABLE Z4
Exemplary floxed Ring2 vector plasmid after recombination
event (pRTx-2846, post-recombination)

| | |
|---|---|
| Name | pRTx-2846_post_recombine |
| Type | Recombinant Plasmid |
| Description | pLox- |
| | Ring2ΔORF::fCMV_EGFP_WPRE_bGH- |
| | pA-Rand200 after recombination by Cre |
| | recombinase |
| Length | 3015 bp |

(SEQ ID NO: 2621)

| | |
|---|---|
| Annotations: | |
| Region/Element | Base range |
| Full CMV promoter | 1-570 |
| Inert 5′UTR | 582-616 |
| eGFP CDS | 617-1336 |
| WPRE | 1340-1931 |
| bGHpA terminator | 1936-2163 |
| sequence | |
| 200 bp random stuffer | 2164-2363 |
| sequence | |
| loxP site | 2368-2401 |
| VL46-002-LY2 gene | 2402-2566 |
| GC-rich region | 2457-2568 |
| VL46-002-LY2 gene | 2571-2991 |
| Promoter | 2590-2833 |
| Initiator element | 2825-2840 |
| 5′UTR conserved domain | 2891-2961 |
| Origin of replication | 2891-2961 |
| Intron 1 | 2903-2991 |

TABLE Z5
Exemplary Cre recombinase expression plasmid (pRTx-2848)

| | |
|---|---|
| Name | pRTx-2848 |
| Type | Plasmid |
| Description | CMV_iCre_pcDNA3.1(+). |
| Length | 6473 bp |

(SEQ ID NO: 2622)

| | |
|---|---|
| Annotations: | |
| Region/Element | Base range |
| CMV enhancer | 235-614 |
| CMV promoter | 615-818 |
| T7 RNA polymerase promoter | 863-880 |
| iCre coding sequence | 964-2019 |
| bGHpA terminator sequence | 2070-2293 |
| Origin of replication | 2962-3097 |
| SV40 polyA sequence | 4146-4267 |
| Lac operon operator (lacO) | 4340-4356 |
| Lac operon promoter (complement) | 4364-4394 |
| C-tag | 4372-4383 |
| Catabolite activator protein binding site | 4409-4430 |
| Origin of replication (complement) | 4718-5306 |
| Ampicillin resistance gene promoter | 6338-6442 |
| (complement) | |

TABLE Z6
Exemplary Ring19 vector plasmid for Bxb1
three-plasmid systems (pRTx-3757)

| | |
|---|---|
| Name | pRTx-3757 |
| Type | Plasmid |
| Description | Ring19 genetic element construct |
| | with Bxb1 recognition sites |
| Length | 5500 bp |

(SEQ ID NO: 2623)

| | |
|---|---|
| Annotations: | |
| Region/Element | Base range |
| Origin of replication (complement) | 1-589 |
| Kanamycin resistance gene CDS | 767-1576 |
| (complement) | |
| attB site | 2118-2163 |
| GC-rich region | 2231-2342 |
| Initiator element | 2515-2530 |
| 5′UTR conserved domain | 2582-2652 |
| Origin of replication | 2582-2652 |
| Intron 1 | 2594-2681 |
| CMV enhancer | 2695-3074 |
| Full CMV promoter | 2710-3279 |
| CMV promoter | 3075-3278 |
| Kozak sequence | 3320-3329 |
| eGFP CDS | 3326-4045 |
| WPRE | 4049-4640 |
| Factor Xa recognition and cleavage | 4520-4531 |
| site (complement) | |
| bGHpA terminator sequence | 4645-4872 |
| 100 bp random stuffer sequence | 4873-4972 |
| attP site | 4981-5038 |
| Lac operon operator | 5123-5139 |
| LacI repressor binding site | 5123-5139 |
| Lac operon promoter (complement) | 5147-5177 |
| CAP binding site | 5192-5213 |

TABLE Z7
Exemplary Ring19 vector plasmid after Bxb1 recombination
event (pRTx-3757, post-recombination)

| | |
|---|---|
| Name | pRTx-3757_post_recombine |
| Type | Recombinant Plasmid |
| Description | Ring19 genetic element construct |
| | after recombination by Bxb1 |
| Length | 2867 bp |

(SEQ ID NO: 2624)

| | |
|---|---|
| Annotations: | |
| Region/Element | Base range |
| GC-rich region | 64-175 |
| Initiator element | 348-363 |
| 5′UTR conserved domain | 415-485 |
| Origin of replication | 415-485 |
| Intron 1 | 427-514 |
| CMV enhancer | 528-907 |
| Full CMV promoter | 543-1112 |
| CMV promoter | 908-1111 |
| Kozak sequence | 1153-1162 |
| eGFP CDS | 1159-1878 |
| WPRE | 1882-2473 |
| Factror Xa recognition and cleavage | 2353-2364 |
| site (complement) | |
| bGHpA terminator sequence | 2478-2705 |
| 100 bp random stuffer sequence | 2706-2805 |
| attR site | 2814-2867 |

TABLE Z8
Exemplary Ring2 vector plasmid for Bxb1
three-plasmid systems (pRTx-3852)

| | |
|---|---|
| Name | pRTx-3852 |
| Type | Plasmid |
| Description | Ring2 genetic element construct |
| | with Bxb1 recognition sites |
| Length | 5576 bp |

(SEQ ID NO: 2625)

| | |
|---|---|
| Annotations: | |
| Region/Element | Base range |
| attB site | 431-480 |
| VL46-002-LY2 gene | 481-645 |
| GC-rich region | 536-647 |
| Promoter | 669-912 |
| Initiator element | 904-919 |
| Origin of replication | 970-1040 |
| 5′UTR conserved domain | 970-1040 |
| Intron 1 | 982-1070 |
| CMV enhancer | 1084-1463 |
| Full CMV promoter | 1099-1668 |
| CMV promoter | 1464-1667 |
| Kozak sequence | 1709-1718 |
| eGFP CDS | 1715-2434 |
| WPRE | 2438-3029 |
| Factor Xa recognition and cleavage site | 2909-2920 |
| (complement) | |
| bGHpA terminator sequence | 3034-3261 |
| 100 bp random stuffer sequence | 3262-3361 |
| attP site | 3370-3427 |
| Lac operon operator | 3512-3528 |
| LacI repressor binding site | 3512-3528 |
| Lac operon promoter (complement) | 3536-3566 |
| CAP binding site | 3581-3602 |
| ColE1/pMB1/pBR322/pUC origin of | 3890-4478 |
| replication (complement) | |
| Kanamycin resistance gene CDS | 4656-5465 |
| (complement) | |

TABLE Z9
Exemplary Ring2 vector plasmid after Bxb1 recombination
event (pRTx-3852, post-recombination)

| | |
|---|---|
| Name | pRTx-3852_post_recombine |
| Type | Recombinant Plasmid |
| Description | Ring2 genetic element construct |
| | after recombination by Bxb1 |
| Length | 2943 bp |

(SEQ ID NO: 2626)

| | |
|---|---|
| Annotations: | |
| Region/Element | Base range |
| VL46-002-LY2 gene | 1-165 |
| GC-rich region | 56-167 |
| Promoter | 189-432 |
| Initiator element | 424-439 |
| Origin of replication | 490-560 |
| 5′UTR conserved domain | 490-560 |
| Intron 1 | 502-590 |
| CMV enhancer | 604-983 |
| Full CMV promoter | 619-1188 |
| CMV promoter | 984-1187 |
| Kozak sequence | 1229-1238 |
| eGFP CDS | 1235-1954 |
| WPRE | 1958-2549 |
| Factor Xa recognition and cleavage | 2429-2440 |
| site (complement) | |
| bGHpA terminator sequence | 2554-2781 |
| 100 bp random stuffer sequence | 2782-2881 |
| attR site | 2890-2943 |

TABLE Z10
Exemplary CMV-Bxb1 recombinase
expression plasmid (pRTx-3542)

| | |
|---|---|
| Name | pRTx-3542 |
| Type | Plasmid |
| Description | Bxb1 integrase expression |
| | plasmid |
| Length | 7003 bp |

(SEQ ID NO: 2627)

| | |
|---|---|
| Annotations: | |
| Region/Element | Base range |
| CMV enhancer | 235-614 |
| CMV promoter | 615-818 |
| T7 RNA polymerase promoter | 863-881 |
| Bxb1 CDS | 934-2490 |
| SV40 nuclear localization signal CDS | 937-957 |
| HA epitope tag CDS | 958-984 |
| bGHpA terminator sequence | 2600-2824 |
| F1 origin of replication | 2870-3298 |
| SV40 promoter | 3312-3641 |
| SV40 origin of replication | 3492-3627 |
| Neomycin/Kanamycin resistance gene CDS | 3708-4502 |
| SV40 pA signal | 4676-4797 |
| LacI repressor binding site | 4870-4886 |
| Lac operon promoter (complement) | 4894-4924 |
| CAP binding site | 4939-4960 |
| ColE1/pMB1/pBR322/pUC origin of | 5248-5836 |
| replication (complement) | |
| Ampicillin resistance (AmpR) gene CDS | 6007-6867 |
| (complement) | |
| AmpR promoter (complement) | 6868-6972 |

### Exemplary NLS_HA_Bxb1 Integrase Amino Acid Sequence (e.g., Encoded by Nucleotides 934-2490 of Table Z10):

(SEQ ID NO: 2628)
MPKKKRKVYPYDVPDYAGSRALVVIRLSRVTDATTSPERQLESCQQLCA
QRGWDVVGVAEDLDVSGAVDPFDRKRRPNLARWLAFEEQPFDVIVAYRV
DRLTRSIRHLQQLVHWAEDHKKLVVSATEAHFDTTTPFAAVVIALMGTV
AQMELEAIKERNRSAAHFNIRAGKYRGSLPPWGYLPTRVDGEWRLVPDP
VQRERILEVYHRVVDNHEPLHLVAHDLNRRGVLSPKDYFAQLQGREPQG
REWSATALKRSMISEAMLGYATLNGKTVRDDDGAPLVRAEPILTREQLE
ALRAELVKTSRAKPAVSTPSLLLRVLFCAVCGEPAYKFAGGGRKHPRYR
CRSMGFPKHCGNGTVAMAEWDAFCEEQVLDLLGDAERLEKVWVAGSDSA
VELAEVNAELVDLTSLIGSPAYRAGSPQREALDARIAALAARQEELEGL
EARPSGWEWRETGQRFGDWWREQDTAAKNTWLRSMNVRLTFDVRGGLTR
TIDFGDLQEYEQHLRLGSVVERLHTGMS

### Exemplary SV40 Nuclear Localization Signal Amino Acid Sequence (e.g., Encoded by Nucleotides 937-957 of Table Z10): PKKKRKV (SEQ ID NO: 2629)

(SEQ ID NO: 2629)
PKKKRKV

### Exemplary HA Epitope Tag Amino Acid Sequence (e.g., Encoded by Nucleotides 958-984 of Table Z10):

(SEQ ID NO: 2504)
YPYDVPDYA

### Two-Plasmid Systems

In some embodiments, a site specific recombinase-based system for producing genetic elements comprises two plasmids: (1) a first plasmid (e.g., a vector plasmid) comprising the sequence of the genetic element, flanked by a pair of recombinase recognition sites; and (2) a second plasmid (e.g., a self-replicating rescue (SRR) plasmid) providing one or more Anelloviridae family viral proteins (e.g., Anellovirus ORF1, ORF3, and/or ORF3 molecules; or CAV VP1, VP2, and/or VP3 molecules), including a capsid protein (e.g., an ORF1 molecule or VP1 molecule, e.g., as described herein). In certain embodiments, the first plasmid further comprises a cassette encoding a site-specific recombinase (e.g., as described herein) capable of recognizing the recombinase recognition sites. In certain embodiments, the second plasmid comprises a cassette encoding a site-specific recombinase (e.g., as described herein) capable of recognizing the recombinase recognition sites. In certain embodiments, the first plasmid and the second plasmid each comprise a cassette encoding a site-specific recombinase (e.g., as described herein) capable of recognizing the recombinase recognition sites. In some embodiments, the ORF1 molecule encoded by one of the plasmids comprises a deletion relative to a wild-type Anellovirus ORF1 protein (e.g., a deletion of all or part of an Anellovirus ORF1 structural arginine-rich region and/or structural C-terminal domain (CTD), e.g., as described herein).

In some embodiments, the portion of the first plasmid flanked by recombination recognition sites comprises, in order: (i) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a nucleic acid sequence encoding a transgene of interest; and (iii) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto.

In some embodiments, the portion of the first plasmid flanked by recombination recognition sites comprises, in order: (i) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; and (iii) a nucleic acid sequence encoding a transgene of interest.

A host cell may comprise the first plasmid and/or the second plasmid. Expression of the site-specific recombinase (e.g., from the cassette in the first plasmid or the cassette in the second plasmid) enables recombination of the first plasmid to produce minicircles comprising the genetic element sequence (which may, in some instances, be converted to cssDNA in the cell). Capsid proteins (e.g., ORF1 molecules or VP1 molecules) can be expressed from the second plasmid, and can then form proteinaceous exteriors (e.g., as described herein) encapsulating the genetic elements (e.g., after conversion to cssDNA), thereby producing Anelloviridae family vector particles. In some embodiments, expression of an ORF2 or ORF3 molecule, or a VP2 or VP3 molecule, from the second plasmid contributes to replication and/or packaging.

Non-limiting examples of first plasmids suitable for use in a two-plasmid recombinase system as described herein are provided in Tables Z11-Z18 below. Tables Z11-Z14 provide exemplary first plasmid (e.g., vector plasmid) sequences for use in a two-plasmid system utilizing Cre recombinase to recombine a genetic element sequence flanked by loxP recombinase recognition sites. Tables Z15-Z18 provide exemplary first plasmid (e.g., vector plasmid) sequences for use in a two-plasmid system utilizing Bxb1 recombinase to recombine attB and attP sites flanking the genetic element sequence.

In some embodiments, a vector plasmid (e.g., suitable for use in a two-plasmid system as described herein) comprises the nucleic acid sequence of a vector plasmid as listed in any of Tables Z11 or Z13 (or an element or subsequence thereof as described in the respective table), or a nucleic acid sequence having at least 75%, 80%, 85%, 90%, 95%, 96%, 97%, 98%, or 99% identity thereto. In some embodiments, a recombined vector plasmid (e.g., a minicircle as described herein) comprises the nucleic acid sequence of a vector plasmid as listed in any of Tables Z12 or Z14 (or an element or subsequence thereof as described in the respective table), or a nucleic acid sequence having at least 75%, 80%, 85%, 90%, 95%, 96%, 97%, 98%, or 99% identity thereto.

In some embodiments, a vector plasmid (e.g., suitable for use in a two-plasmid system as described herein) comprises the nucleic acid sequence of a vector plasmid as listed in any of Tables Z15 or Z17 (or an element or subsequence thereof as described in the respective table), or a nucleic acid sequence having at least 75%, 80%, 85%, 90%, 95%, 96%, 97%, 98%, or 99% identity thereto. In some embodiments, a recombined vector plasmid (e.g., a minicircle as described herein) comprises the nucleic acid sequence of a vector plasmid as listed in any of Tables Z16 or Z18 (or an element or subsequence thereof as described in the respective table), or a nucleic acid sequence having at least 75%, 80%, 85%, 90%, 95%, 96%, 97%, 98%, or 99% identity thereto.

In any of the embodiments, the coding sequence of the transgene (e.g., eGFP, lacZ) in the vector plasmids may be replaced with any transgene, e.g., a transgene encoding an exogenous effector, e.g., a therapeutic effector (e.g., as described herein).

In some embodiments, the second plasmid comprises the sequence of an exemplary SRR plasmid as described in the SRR constructs section below.

TABLE Z11
Exemplary Ring19 vector plasmid encoding
Cre recombinase (pRTx-3975)

| | |
|---|---|
| Name | pRTx-3975 |
| Type | Plasmid |
| Description | Ring19iCREM |
| Length | 6246 bp |

(SEQ ID NO: 2630)

| | |
|---|---|
| Annotations: | |
| Region/Element | Base range |
| pUC origin of replication (complement) | 1-674 |
| Kanamycin resistance (KanR) CDS (complement) | 826-1635 |
| Lox71 (loxP site) | 2180-2213 |
| GC rich region | 2277-2388 |
| Initiator element | 2561-2576 |
| Origin of replication | 2628-2698 |
| 5′UTR conserved domain | 2628-2698 |
| Intron 1 | 2640-2727 |
| LacZa CDS (complement) | 2866-3189 |
| LacZa multiple cloning site | 3116-3172 |
| Lac operon operator | 3209-3225 |
| Lac operon promoter (complement) | 3227-3266 |
| C tag | 3241-3252 |
| CAP binding site (complement) | 3278-3299 |
| CAP binding site | 3278-3299 |
| lox66 (loxP site) | 3416-3449 |
| CMV enhancer | 3506-3885 |
| CMV promoter | 3886-4089 |
| Insert sequence | 4085-5408 |
| T7 RNA polymerase promoter | 4134-4152 |
| Chimeric human beta-globin and immunoglobulin | 4448-4580 |
| heavy chain intron | |
| bGHpA terminator sequence | 5479-5703 |
| F1 origin of replication | 5749-5876 |
| LacI repressor binding site (complement) | 5926-5948 |
| Lac operon operator | 5928-5944 |
| Lac operon promoter (complement) | 5952-5982 |
| C tag | 5960-5971 |
| CAP binding site (complement) | 5997-6018 |
| CAP binding site | 5997-6018 |

TABLE Z12
Exemplary Ring19 vector plasmid encoding Cre recombinase
after recombination event (pRTx-3975, post-recombination)

| | |
|---|---|
| Name | pRTx-3975_post_recombine |
| Type | Circular DNA |
| Description | Ring19iCREM post recombination |
| Length | 1236 bp |

(SEQ ID NO: 2631)

| | |
|---|---|
| Annotations: | |
| Region/Element | Base range |
| GC rich region | 1-112 |
| Initiator element | 285-300 |
| Origin of replication | 352-422 |
| 5′UTR conserved domain | 352-422 |
| Intron 1 | 364-451 |
| LacZa CDS (complement) | 590-913 |
| LacZa multiple cloning site | 840-896 |
| Lac operon operator | 933-949 |
| Lac operon promoter (complement) | 951-990 |
| C tag | 965-976 |
| CAP binding site (complement) | 1002-1023 |
| Cap binding site | 1002-1023 |
| loxP site | 1140-1173 |

TABLE Z13
Exemplary Ring2 vector plasmid encoding
Cre recombinase (pRTx-3974)

| | |
|---|---|
| Name | pRTx-3974 |
| Type | Plasmid |
| Description | Ring2iCREM |
| Length | 6674 bp |

(SEQ ID NO: 2632)

| | |
|---|---|
| Annotations: | |
| Region/Element | Base range |
| Origin of replication (complement) | 272-860 |
| Kanamycin Resistance (KanR) CDS | 1031-1840 |
| (complement) | |
| T7 RNA polymerase promoter | 2345-2363 |
| Lox71 (loxP site) | 2412-2445 |
| Ring2 NCR | 2446-3035 |
| VL46-002-LY2 gene | 2446-2610 |
| GC-rich region | 2501-2612 |
| HP3 | 2532-2552 |
| VL46-002-LY2 gene | 2615-3035 |
| Promoter | 2634-2877 |
| Initiator element | 2869-2884 |
| Origin of replication | 2935-3005 |
| 5′UTR conserved domain | 2935-3005 |
| Intron 1 | 2947-3035 |
| LacZa CDS (complement) | 3174-3497 |
| LacZa multiple cloning site | 3424-3480 |
| Lac operon operator | 3517-3533 |
| Lac operon promoter (complement) | 3535-3574 |
| Lac operon promoter (complement) | 3541-3571 |
| C tag | 3549-3560 |
| CAP binding site (complement) | 3586-3607 |
| CAP binding site | 3586-3607 |
| Lox66 (loxP site) | 3724-3757 |
| CMV enhancer | 4177-4556 |
| CMV promoter | 4557-4760 |
| Insert sequence | 4756-6079 |
| T7 RNA polymerase promoter | 4805-4823 |
| Chimeric human beta-globin and | 5119-5251 |
| immunoglobulin heavy chain intron | |
| bGHpA terminator sequence | 6150-6374 |
| Origin of replication | 6420-6547 |
| LacUV5 promoter (complement) | 6589-6619 |
| C tag | 6597-6608 |
| CAP binding site (complement) | 6634-6655 |
| CAP binding site | 6634-6655 |

TABLE Z14
Exemplary Ring2 vector plasmid encoding Cre recombinase
after recombination event (pRTx-3974, post-recombination)

| | |
|---|---|
| Name | pRTx-3974_post_recombine |
| Type | Circular DNA |
| Description | Ring2iCREM post recombination |
| Length | 1312 bp |

(SEQ ID NO: 2633)

| | |
|---|---|
| Annotations: | |
| Region/Element | Base range |
| Ring2 NCR | 1-590 |
| VL46-002-LY2 gene | 1-165 |
| GC rich region | 56-167 |
| HP3 | 87-107 |
| VL46-002-LY2 gene | 170-590 |
| Promoter | 189-432 |
| Initiator element | 424-439 |
| Origin of replication | 490-560 |
| 5′UTR conserved domain | 490-560 |
| Intron 1 | 502-590 |
| LacZa CDS (complement) | 729-1052 |
| LacZa multiple cloning site | 979-1035 |
| Lac operon operator | 1072-1088 |
| LacI repressor binding site | 1072-1088 |
| Lac operon promoter (complement) | 1090-1129 |
| Lac operon promoter (complement) | 1096-1126 |
| C tag | 1104-1115 |
| CAP binding site (complement) | 1141-1162 |
| CAP binding site | 1141-1162 |
| LoxP site | 1279-1312 |

TABLE Z15
Exemplary Ring19 vector plasmid encoding
Bxb1 recombinase (pRTx-3900)

| | |
|---|---|
| Name | pRTx-3900 |
| Type | Plasmid |
| Description | Ring19 genetic element construct |
| | with Bxb1 recognition sites and |
| | Bxb1 expression cassette |
| Length | 8345 bp |

(SEQ ID NO: 2634)
Annotations:

| Region/Element | Base range |
|---|---|
| ColE1/pMB1/pBR322/pUC origin of | 1-589 |
| replication (complement) | |
| Kanamycin resistance gene CDS | 767-1576 |
| (complement) | |
| attB site | 2118-2163 |
| GC-rich region | 2231-2342 |
| Initiator element | 2515-2530 |
| 5′UTR conserved domain | 2582-2652 |
| Intron 1 | 2594-2681 |
| Full CMV promoter | 2710-3279 |
| eGFP CDS | 3326-4045 |
| WPRE | 4049-4640 |
| bGHpA terminator sequence | 4645-4872 |
| 100 bp random stuffer sequence | 4873-4972 |
| attP site | 4981-5038 |
| CMV enhancer | 5098-5477 |
| CMV promoter | 5478-5681 |
| T7 RNA polymerase promoter | 5726-5744 |
| Insert sequence | 5774-7481 |
| Chimeric human beta-globin and | 6047-6179 |
| immunoglobulin heavy chain intron | |
| bGHpA terminator sequence | 7575-7799 |
| Chimeric human beta-globin and | 7781-7799 |
| immunoglobulin heavy chain intron | |
| EAM051 (complement) | |
| Chimeric human beta-globin and | 7798-7818 |
| immunoglobulin heavy chain intron | |
| S361 (complement) | |
| F1 origin of replication | 7845-7916 |
| Chimeric human beta-globin and | 7957-7979 |
| immunoglobulin heavy chain intron | |
| S120 (complement) | |
| LacI repressor binding site | 7968-7984 |
| Lac operon operator | 7968-7984 |
| Lac operon promoter (complement) | 7992-8022 |
| CAP binding site | 8037-8058 |

### Exemplary Kanamycin Resistance Amino Acid Sequence (e.g., Encoded by Nucleotides Complement (7501-8310) of Table Z15):

(SEQ ID NO: 2635)
MSHIQRETSRPRLNSNMDADLYGYKWARDNVGQSGATIYRLYGKPDAPE
LFLKHGKGSVANDVTDEMVRLNWLTEFMPLPTIKHFIRTPDDAWLLTTA
IPGKTAFQVLEEYPDSGENIVDALAVFLRRLHSIPVCNCPFNSDRVFRL
AQAQSRMNNGLVDASDFDDERNGWPVEQVWKEMHKLLPFSPDSVVTHGD
FSLDNLIFDEGKLIGCIDVGRVGIADRYQDLAILWNCLGEFSPSLQKRL
FQKYGIDNPDMNKLQFHLMLDEFF

TABLE Z16
Exemplary Ring19 vector plasmid encoding Bxb1 recombinase
after recombination event (pRTx-3900, post-recombination)

| | |
|---|---|
| Name | pRTx-3900_post_recombine |
| Type | Recombinant Plasmid |
| Description | Ring19 genetic element construct |
| | after recombination by Bxb1 |
| Length | 2867 bp |

(SEQ ID NO: 2636)
Annotations:

| Region/Element | Base range |
|---|---|
| GC-rich region | 64-175 |
| Initiator element | 348-363 |
| 5′UTR conserved domain | 415-485 |
| Origin of replication | 415-485 |
| Intron 1 | 427-514 |
| CMV enhancer | 528-907 |
| Full CMV promoter | 543-1112 |
| CMV promoter | 908-1111 |
| Kozak sequence | 1153-1162 |
| eGFP CDS | 1159-1878 |
| WPRE | 1882-2473 |
| Factror Xa recognition and cleavage | 2353-2364 |
| site (complement) | |
| bGHpA terminator sequence | 2478-2705 |
| 100 bp random stuffer sequence | 2706-2805 |
| attR site | 2814-2867 |

TABLE Z17
Exemplary Ring2 vector plasmid encoding
Bxb1 recombinase (pRTx-3901)

| | |
|---|---|
| Name | pRTx-3901 |
| Type | Plasmid |
| Description | Ring2 genetic element construct |
| | with Bxb1 recognition sites and |
| | Bxb1 expression cassette |
| Length | 8421 bp |

(SEQ ID NO: 2637)
Annotations:

| Region/Element | Base range |
|---|---|
| attB site | 431-480 |
| VL46-002-LY2 gene | 481-645 |
| GC-rich region | 536-647 |
| Promoter | 669-912 |
| Full CMV promoter | 1099-1668 |
| Kozak sequence | 1709-1718 |
| eGFP CDS | 1715-2434 |
| WPRE | 2438-3029 |
| bGHpA terminator sequence | 3034-3261 |
| 100 bp random stuffer sequence | 3262-3361 |
| attP site | 3370-3427 |
| CMV enhancer | 3487-3866 |
| CMV promoter | 3867-4070 |
| T7 RNA polymerase promoter | 4115-4133 |
| Insert sequence | 4163-5870 |
| Chimeric human beta-globin and | 4436-4568 |
| immunoglobulin heavy chain intron | |
| bGHpA terminator sequence | 5964-6188 |
| F1 origin of replication | 6234-6305 |
| LacI repressor binding site | 6357-6373 |
| Lac operon operator | 6357-6373 |
| Lac operon promoter (complement) | 6381-6411 |
| CAP binding site | 6426-6447 |
| ColE1/pMB1/pBR322/pUC origin of | 6735-7323 |
| replication (complement) | |
| Kanamycin resistance gene CDS | 7501-8310 |
| (complement) | |

TABLE Z18
Exemplary Ring2 vector plasmid encoding Bxb1 recombinase
after recombination event (pRTx-3901, post-recombination)

| | |
|---|---|
| Name | pRTx-3901_post_recombine |
| Type | Recombinant Plasmid |
| Description | Ring2 genetic element construct |
| | after recombination by Bxb1 |
| Length | 2943 bp |

(SEQ ID NO: 2638)
Annotations:

| Region/Element | Base range |
|---|---|
| VL46-002-LY2 gene | 1-165 |
| GC-rich region | 56-167 |
| Promoter | 189-432 |
| Initiator element | 424-439 |
| Origin of replication | 490-560 |
| 5′UTR conserved domain | 490-560 |
| Intron 1 | 502-590 |
| CMV enhancer | 604-983 |
| Full CMV promoter | 619-1188 |
| CMV promoter | 984-1187 |
| Kozak sequence | 1229-1238 |
| eGFP CDS | 1235-1954 |
| WPRE | 1958-2549 |
| Factor Xa recognition and cleavage | 2429-2440 |
| site (complement) | |
| bGHpA terminator sequence | 2554-2781 |
| 100 bp random stuffer sequence | 2782-2881 |
| attR site | 2890-2943 |

### Self-Replicating Rescue (SRR) Constructs (e.g., SRR Plasmids)

The site-specific recombinase-based systems for producing circular double-stranded genetic elements (e.g., minicircles) can include, in some embodiments, a rescue construct providing one or more Anelloviridae family viral proteins, or functional fragments or variants thereof (e.g., one or more of Anellovirus ORF1, ORF2, and/or ORF3 molecules; or one or more of CAV VP1, VP2, and/or VP3 molecules). In some embodiments, the ORF1 molecule comprises a deletion relative to a wild-type Anellovirus ORF1 protein (e.g., a deletion of all or part of an Anellovirus ORF1 structural arginine-rich region and/or structural C-terminal domain (CTD), e.g., as described herein). In some embodiments, the rescue construct is capable of self-replicating in a host cell (e.g., a self-replicating rescue (SRR) plasmid).

In some embodiments, the rescue construct includes an expression cassette comprising the protein coding sequence of an Anelloviridae family virus (e.g., an Anellovirus or CAV as described herein), or a sequence having at least 75%, 80%, 85%, 90%, 95%, 96%, 97%, 98%, or 99% identity thereto. In some embodiments, the rescue construct includes a sequence encoding a replication protein (e.g., a large T antigen, a PCV Rep, or a PCV Rep′), or a sequence having at least 75%, 80%, 85%, 90%, 95%, 96%, 97%, 98%, or 99% identity thereto. In some embodiments, the rescue construct includes an exogenous origin of replication (e.g., a viral origin, e.g., an SV40, PCV, or AAV origin). In certain embodiments, the replication protein coding sequence is downstream of an internal ribosome entry site (IRES) positioned downstream of the expression cassette comprising the protein coding sequence of the Anelloviridae family virus. In certain embodiments, the replication protein coding sequence is comprised in a separate cassette from the expression cassette comprising the protein coding sequence of the Anelloviridae family virus. In some embodiments, the rescue construct further comprises one or more additional expression cassettes, e.g., encoding a site-specific recombinase, an Anelloviridae family viral protein (e.g., an Anellovirus ORF1 molecule or a CAV VP1 molecule), a replication protein and/or viral origin (e.g., as described herein), or another transgene of interest.

In some embodiments, the rescue construct comprises an expression cassette comprising the full protein-coding region of an Anelloviridae family viral genome (e.g., as described herein). In some embodiments, the rescue construct comprises an expression cassette comprising a protein-coding region of an Anelloviridae family viral genome (e.g., as described herein) comprising one or more mutations relative to the corresponding wild-type sequence.

In certain embodiments, the expression cassette comprises a mutation that prevents expression of a functional ORF1 molecule (e.g., a nonsense mutation, deletion, substitution, replacement, or frameshift) from the protein-coding region. In some embodiments, the mutation is one or more internal stop codons (e.g., TAA) in the ORF1 coding sequence (e.g., the nucleic acid sequence encoding SEQ ID NO: 2696). In some embodiments, the one or more stop codons do not affect the expression of ORF1/1 and other splice variants from the rescue construct. In other embodiments, the expression cassette encodes a functional ORF1 molecule (e.g., a wild-type ORF1 molecule as described herein), or an amino acid sequence having at least 75%, 80%, 85%, 90%, 95%, 96%, 97%, 98%, or 99% identity thereto.

In some embodiments, the rescue construct comprises an ORF1/1 protein coding region.

In some embodiments, the rescue construct comprises a single expression cassette (e.g., as described above). In some embodiments, the rescue construct comprises two expression cassettes. In some embodiments, the rescue construct comprises three expression cassettes. In some embodiments, the rescue construct comprises four expression cassettes. In some embodiments, the rescue construct comprises five or more expression cassettes. The exemplary expression cassettes described herein can be positioned in any order within the rescue construct. In some embodiments, one or more of the expression cassettes is in the opposite orientation relative to one or more of the other expression cassettes. In other embodiments, all of the expression cassettes in a rescue construct are in the same orientation relative to each other.

In some embodiments, the expression cassette includes a promoter. Exemplary promoters that can be included in an expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In certain embodiments, the expression cassette includes a T cell promoter, i.e., a promoter that is capable of driving expression in a T cell (e.g., an MNDU3, SFFV, MSCV, Distal Lek (dLck), CD36, EF1a, RPBSA, or Cbh promoter). In embodiments, the expression cassette comprises an MNDU3 promoter. In embodiments, the MNDU3 promoter comprises at least 75%, 80%, 85%, 90%, 95%, 96%, 97%, 98%, 99%, or 100% sequence identity to the nucleic acid sequence of SEQ ID NO: 2784. In embodiments, the expression cassette comprises a SFFV promoter. In embodiments, the SFFV promoter comprises at least 75%, 80%, 85%, 90%, 95%, 96%, 97%, 98%, 99%, or 100% sequence identity to the nucleic acid sequence of SEQ ID NO: 2786. In embodiments, the expression cassette comprises a MSCV promoter. In embodiments, the MSCV promoter comprises at least 75%, 80%, 85%, 90%, 95%, 96%, 97%, 98%, 99%, or 100% sequence identity to the nucleic acid sequence of SEQ ID NO: 2785.

In some embodiments, the expression cassette further comprises an enhancer, e.g., as described herein.

In some embodiments, the promoter is Cbh (CMV early enhancer fused to modified R-actin promoter).

In some embodiments, the promoter is active in T cells, e.g., MOLT-4 cells. In some embodiments, the promoter is MNDU3 (modified murine leukemia virus long terminal repeat promoter). In some embodiments, the promoter is Distal Lek (dLck). In some embodiments, the promoter is CD36. In some embodiments, the promoter is EF1a. In some embodiments, the promoter is MSCV (murine stem cell virus promoter). In some embodiments, the promoter is synthetic RPBSA promoter. In some embodiments, the promoter is SFFV (spleen focus forming virus promoter).

In some embodiments, the rescue construct does not contain an Anellovirus NCR sequence (e.g. does not contain an Anellovirus 5′ NCR sequence and/or an Anellovirus 3′ NCR sequence). In some embodiments, the rescue construct does not comprise an Anellovirus 5′ UTR conserved domain. In some embodiments, the rescue construct does not comprise an Anellovirus GC-rich region. In some embodiments, the rescue construct does not comprise an Anellovirus NCR intron. In some embodiments, the rescue construct does not comprise an Anellovirus NCR exon. In some embodiments, the rescue construct does not comprise an Anellovirus NCR intron or exon. In some embodiments, the rescue construct does not contain Anellovirus sequences homologous to the vector plasmid (e.g., a contiguous sequence of at least 10, 20, 30, 40, 50, 60, 70, 80, 90, 100, 125, 150, 175, 200, 250, 300, 400, or 500 nucleotides having sequence identity to any Anellovirus sequence of the same length in the vector plasmid). In some embodiments, the rescue construct contains a native kozak sequence of an Anellovirus ORF2 or VP2 coding sequence. Exemplary SRR plasmids that can, in some embodiments, be used in a site-specific recombinase system as described herein (e.g., a two-plasmid system or three-plasmid system as described herein) are provided in Tables W1-W8. In some embodiments, a rescue construct (e.g., suitable for use in a two- or three-plasmid system as described herein) comprises the nucleic acid sequence of an SRR plasmid as listed in Table WI (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W2 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W3 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX4 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX5 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX6 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX7 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX8 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WX9 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table WI (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W3A (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W4 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W5 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W6 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W7 (or an element or subsequence thereof as described in the respective table), or a nucleic acid sequence having at least 75%, 80%, 85%, 90%, 95%, 96%, 97%, 98%, or 99% identity thereto. In some embodiments, a rescue construct (e.g., suitable for use in a two- or three-plasmid system as described herein) comprises the nucleic acid sequence of an SRR plasmid as listed in Table W8 (or an element or subsequence thereof as described in the respective table), or a nucleic acid sequence having at least 75%, 80%, 85%, 90%, 95%, 96%, 97%, 98%, or 99% identity thereto.

TABLE W1
Exemplary Ring19 SRR plasmid (pRTx-3525)

| | |
|---|---|
| Name | pRTx-3525 |
| Type | Plasmid |
| Description | phEF1a_Ring19-UTR- |
| | FullORF_SVLT_SV40ori. |
| Length | 9391 bp |

(SEQ ID NO: 2000)
Annotations:

| Region/Element | Base range |
|---|---|
| pHEf1A promoter | 436-1609 |
| EF-1-alpha core promoter | 457-668 |
| EF-1-alpha intron A | 669-1607 |
| Initiator element | 1614-1618 |
| Exon 1 | 1625-1681 |
| 5′ UTR conserved domain | 1670-1740 |
| Kozak sequence | 1676-1681 |
| Intron 1 | 1682-1769 |
| ORF2/3 coding sequence | 1770-2056, 3756-4131 |
| ORF2/2 coding sequence | 1770-2056, 3629-3902 |
| ORF2 coding sequence | 1770-2060 |
| ORF1 coding sequence | 1952-3919 |
| ORF1/1 coding sequence | 1952-2056, 3629-3919 |
| ORF1/2 coding sequence | 1952-2056, 3756-3902 |
| EMCV internal ribosome entry site (IRES) | 4203-4789 |
| SV40 Large T Antigen coding sequence | 4790-6916 |
| SV40 polyA sequence (complement) | 6947-7068 |
| SV40 origin of replication | 7108-7243 |
| LacI repressor protein binding site | 7325-7341 |
| (complement) | |
| Lac operon promoter (complement) | 7349-7379 |
| CAP binding site (complement) | 7394-7415 |
| pUC origin of replication (complement) | 7644-8317 |
| ColE1/pMB1/pBR322/pUC origin of | 7703-8291 |
| replication | |
| Aminoglycoside phosphotransferase (Kan/ | 8469-9278 |
| G418 resistance protein) coding sequence | |
| (complement) | |

### Exemplary SV40 Large T Antigen Amino Acid Sequence (e.g., Encoded by Nucleotides 4790-6916 of Table W1):

(SEQ ID NO: 2001)
MDKVLNREESLQLMDLLGLERSAWGNIPLMRKAYLKKCKEFHPDKGGDE
EKMKKMNTLYKKMEDGVKYAHQPDFGGFWDATEIPTYGTDEWEQWWNAF
NEENLFCSEEMPSSDDEATADSQHSTPPKKKRKVEDPKDFPSELLSFLS
HAVFSNRTLACFAIYTTKEKAALLYKKIMEKYSVTFISRHNSYNHNILF
FLTPHRHRVSAINNYAQKLCTFSFLICKGVNKEYLMYSALTRDPFSVIE
ESLPGGLKEHDFNPEEAEETKQVSWKLVTEYAMETKCDDVLLLLGMYLE
FQYSFEMCLKCIKKEQPSHYKYHEKHYANAAIFADSKNQKTICQQAVDT
VLAKKRVDSLQLTREQMLTNRFNDLLDRMDIMFGSTGSADIEEWMAGVA
WLHCLLPKMDSVVYDFLKCMVYNIPKKRYWLFKGPIDSGKTTLAAALLE
LCGGKALNVNLPLDRLNFELGVAIDQFLVVFEDVKGTGGESRDLPSGQG
INNLDNLRDYLDGSVKVNLEKKHLNKRTQIFPPGIVTMNEYSVPKTLQA
RFVKQIDFRPKDYLKHCLERSEFLLEKRIIQSGIALLLMLIWYRPVAEF
AQSIQSRIVEWKERLDKEFSLSVYQKMKFNVAMGIGVLDWLRNSDDDDE
DSQENADKNEDGGEKNMEDSGHETGIDSQSQGSFQAPQSSQSVHDHNQP
YHICRGFTCFKKPPTPPPEPET

TABLE W2
Exemplary Ring19 SRR plasmid engineered to remove homology
to WT Anellovirus NCR sequences (pRTx-3856)

| | |
|---|---|
| Name | pRTx-3856 |
| Type | Plasmid |
| Description | pSRR::hEF1a_Ring19- |
| | Kozak-FullORF |
| Length | 9296 bp |

(SEQ ID NO: 2002)
Annotations:

| Region/Element | Base range |
|---|---|
| HEf1A promoter | 447-1620 |
| HEf1A inton A | 680-1618 |
| Inert 5′UTR | 1624-1658 |
| Kozak sequence | 1659-1664 |
| Ring19 ORF2 CDS | 1665-1955 |
| Ring19 ORF2/2 CDS | 1665-1951, 3524-3797 |
| Ring19 ORF 2/3 CDS | 1665-1951, 3651-4026 |
| Ring19 ORF1 CDS | 1847-3814 |
| EMCV internal ribosome entry site (IRES) | 4108-4694 |
| SV40 Large T Antigen coding sequence | 4695-6821 |
| SV40 Large T Antigen nuclear localization | 5070-5090 |
| sequence | |
| SV40 polyA (complement) | 6852-6973 |
| SV40 polyA | 6852-6973 |
| SV40 polyA terminator | 6899-6973 |
| SV40 origin of replication | 7013-7148 |
| LacI binding site (complement) | 7228-7250 |
| Lac operon operator | 7230-7246 |
| LacI binding site | 7230-7246 |
| Lac operon promoter (complement) | 7254-7284 |
| C tag | 7262-7273 |
| CAP binding site (complement) | 7299-7320 |
| CAP binding site | 7299-7320 |
| Origin of replication (complement) | 7549-8222 |
| ColE1/pMB1/pBR322/pUC origin of | 7608-8196 |
| replication (complement) | |
| RNAi transcript | 8050-8157 |
| Kanamycin/G418 resistance CDS | 8374-9183 |
| (complement) | |

TABLE W3
Exemplary Ring2 SRR plasmid (pRTx-3853)

| | |
|---|---|
| Name | pRTx-3853 |
| Type | Plasmid |
| Description | pSRR::hEF1a_Ring2-FullORF |
| Length | 9418 bp |

(SEQ ID NO: 2003)
Annotations:

| Region/Element | Base range |
|---|---|
| HEf1A intron A | 669-1607 |
| Origin of replication | 1669-1739 |
| Intron 1 | 1681-1769 |
| Ring2 ORF 2/2 CDS | 1770-2065, 3620-3935 |
| Ring2 ORF2 CDS | 1770-2069 |
| Ring2 ORF1 CDS | 1958-3958 |
| Ring2 arginine rich domain | 1958-2071 |
| Jelly roll domain | 2072-2695 |
| HVD motif | 2696-3079 |
| N22 motif | 3080-3568 |
| C-terminal domain | 3569-3955 |
| ORFs | 3787-3932 |
| EMCV internal ribosome entry site | 4230-4816 |
| (IRES) | |
| SV40 Large T Antigen nuclear | 5192-5212 |
| localization | |
| SV40 polyA signal (complement) | 6974-7095 |
| SV40 polyA signal | 6974-7095 |
| SV40 origin of replication | 7135-7270 |
| LacI repressor binding site | 7350-7372 |
| (complement) | |
| Lac operon promoter (complement) | 7376-7406 |
| CAP binding site (complement) | 7421-7442 |
| ColE1/pMB1/pBR322/pUC origin of | 7730-8318 |
| replication (complement) | |
| Kanamycin/G418 resistance CDS | 8496-9305 |
| (complement) | |

TABLE WX4
Exemplary Ring19 SRR plasmid (pRTx-4511)

| | |
|---|---|
| Name | pRTx-4511 |
| Type | Plasmid |
| Description | MNDU3_Ring19-UTR- |
| | FullORF_SVLT_SV40ori |
| Length | 8612 bp |

(SEQ ID NO: 2004)
Annotations:

| Region/Element | Base range |
|---|---|
| MNDU3 promoter | 436-830 |
| Initiator element | 835-839 |
| 5′ UTR conserved domain | 891-961 |
| Intron 1 | 903-990 |
| Ring19 ORF2/3 | 991-1277, 2977-3352 |
| Ring19 ORF2/2 | 991-1277, 2850-3123 |
| Ring19 ORF2 | 991-1281 |
| Ring19 ORF1 | 1173-3140 |
| Ring19 ORF1/1 | 1173-1277, 2850-3140 |
| Ring19 ORF1/2 | 1173-1277, 2977-3123 |
| Internal ribosome entry site (IRES) | 3424-4010 |
| SV40 large T antigen | 4011-6137 |
| SV40 polyadenylation signal | 6168-6289 |
| SV40 origin of replication | 6329-6464 |
| Lac operator | 6546-6562 |
| Promoter for the E. coli lac operon | 6570-6600 |
| CAP binding site | 6615-6636 |
| High copy number ColE1/pMB1/pBR322/ | 6924-7512 |
| pUC origin of replication | |

TABLE WX5
Exemplary Ring19 SRR plasmid with NCR intron 1 deletion

| | |
|---|---|
| Name | pRTx-3696 |
| Type | Plasmid |
| Description | hEF1a_Ring19-Exon1- |
| | FullORF_IRES_SVLT_SV40ori |
| Length | 9292 bp |

(SEQ ID NO: 2005)
Annotations:

| Region/Element | Base range |
|---|---|
| pHEf1A promoter | 436-1609 |
| EF-1-alpha intron A | 669-1607 |
| Exon 1 | 1614-1670 |
| Kozak sequence | 1665-1670 |
| Ring19 ORF2 | 1671-1961 |
| Ring19 ORF2/2 | 1671-1957, 3530-3803 |
| Ring19 ORF2/3 | 1671-1957, 3657-4032 |
| Ring19 ORF1 | 1853-3820 |
| Internal ribosome entry site (IRES) | 4104-4690 |
| SV40 large T antigen | 4691-6817 |
| SV40 nuclear localization signal | 5066-5086 |
| SV40 polyadenylation signal | 6848-6969 |
| SV40 origin of replication | 7009-7144 |
| Lac operon operator | 7226-7242 |
| Promoter for the E. coli lac operon | 7250-7280 |
| (complement) | |
| C-tag | 7258-7269 |
| CAP binding site | 7295-7316 |
| High-copy-number ColE1/pMB1/ | 7604-8192 |
| pBR322/pUC origin of replication | |
| (complement) | |
| RNAI transcript | 8046-8153 |
| Kanamycin resistance coding | 8370-9179 |
| sequence (complement) | |

TABLE WX6
Exemplary Ring19 SRR plasmid with
stop codon in ORF1 coding sequence

| | |
|---|---|
| Name | pRTx-3775 |
| Type | Plasmid |
| Description | hEF1a_Ring19- |
| | SRR_FullORF-ORF1KO |
| Length | 9391 bp |

(SEQ ID NO: 2006)
Annotations:

| Region/Element | Base range |
|---|---|
| pHEf1A promoter | 436-1609 |
| EF-1-alpha intron A | 669-1607 |
| 5′ UTR conserved domain | 1670-1740 |
| Intron 1 | 1682-1769 |
| Ring19 ORF2/3 | 1770-2056, 3756-4131 |
| Ring19 ORF2/2 | 1770-2056, 3629-3902 |
| Ring19 ORF2 | 1770-2060 |
| Ring19 ORF1 | 1952-3919 |
| Internal ribosomal entry site (IRES) | 4203-4789 |
| SV40 large T antigen | 4790-6916 |
| SV40 nuclear localization sequence | 5165-5185 |
| SV40 polyadenylation signal | 6947-7068 |
| SV40 origin of replication | 7108-7243 |
| Lac operon operator | 7325-7341 |
| Promoter for the E. coli lac operon | 7349-7379 |
| (complement) | |
| C-tag | 7357-7368 |
| CAP binding site | 7394-7415 |
| pUC origin (complement) | 7644-8317 |
| High-copy-number ColE1/pMB1/pBR322/ | 7703-8291 |
| pUC origin of replication (complement) | |
| RNAI transcript | 8145-8252 |
| Kanamycin resistance coding sequence | 8469-9278 |
| (complement) | |

TABLE WX7
Exemplary Ring19 SRR plasmid encoding ORF1/1

| | |
|---|---|
| Name | pRTx-4534 |
| Type | Plasmid |
| Description | |
| Length | 6536 bp |

(SEQ ID NO: 2007)
Annotations:

| Region/Element | Base range |
|---|---|
| pHEf1A promoter | 174-1347 |
| G−>T mutation | 233 |
| EF-1-alpha intron A | 407-1345 |
| Inert 5′ UTR | 1348-1378 |
| Ring19 ORF1/1 | 1383-1778 |
| Inert 3′ UTR | 1782-1805 |
| Internal ribosome entry site (IRES) | 1810-2396 |
| SV40 large T antigen | 2397-4523 |
| SV40 nuclear localization sequence | 2772-2792 |
| SV40 polyadenylation signal (complement) | 4554-4675 |
| SV40 origin of replication | 4715-4850 |
| High-copy-number ColE1/pMB1/pBR322/pUC | 4937-5525 |
| origin of replication (complement) | |
| Kanamycin resistance coding sequence | 5703-6512 |
| (complement) | |

TABLE WX8
Exemplary Ring2 SRR plasmid with stop
codon in ORF1 coding sequence

| | |
|---|---|
| Name | pRTx-3776 |
| Type | Plasmid |
| Description | hEF1a_Ring2- |
| | SRR_FullORF-ORF1KO |
| Length | 9418 bp |

(SEQ ID NO: 2008)
Annotations:

| Region/Element | Base range |
|---|---|
| pHEf1A | 436-1609 |
| EF-1-alpha intron A | 669-1607 |
| Intron 1 | 1681-1769 |
| Ring2 ORF2 | 1770-2069 |
| Ring2 ORF2/2 | 1770-2065, 3620-3935 |
| Ring2 ORF1 | 1958-3958 |
| Ring2 ORF1 arginine-rich domain | 1958-2071 |
| C-terminal domain | 3569-3955 |
| Internal ribosomal entry site (IRES) | 4230-4816 |
| SV40 large T antigen | 4817-6943 |
| SV40 nuclear localization sequence | 5192-5212 |
| KT3 epitope tag | 6908-6940 |
| SV40 polyadenylation signal | 6974-7095 |
| SV40 origin | 7135-7212 |
| Lac operon operator | 7352-7368 |
| Lac operon promoter (complement) | 7376-7406 |
| C-tag | 7384-7395 |
| CAP binding site | 7421-7442 |
| High-copy-number ColE1/pMB1/pBR322/ | 7730-8318 |
| pUC origin of replication (complement) | |
| RNAI transcript | 8172-8279 |
| Kanamycin resistance coding sequence | 8496-9305 |
| (complement) | |

TABLE WX9
Exemplary Ring2 SRR plasmid encoding ORF1/1

| | |
|---|---|
| Name | pRTx-4463 |
| Type | Plasmid |
| Description | |
| Length | 6588 bp |

(SEQ ID NO: 2009)
Annotations:

| Region/Element | Base range |
|---|---|
| pHEf1A promoter | 174-1347 |
| G−>T mutation | 233 |
| EF-1-alpha intron A | 407-1345 |
| Inert 5′UTR | 1348-1378 |
| Ring2 ORF1/1 | 1383-1829 |
| Inert 3′UTR | 1834-1857 |
| Internal ribosome entry site (IRES) | 1862-2448 |
| SV40 large T antigen | 2449-4575 |
| SV40 nuclear localization signal | 2824-2844 |
| SV40 polyadenylation signal (complement) | 4606-4727 |
| SV40 origin of replication | 4767-4902 |
| High-copy-number ColE1/pMB1/pBR322/pUC | 4989-5577 |
| origin of replication (complement) | |
| Kanamycin resistance coding sequence | 5755-6564 |
| (complement) | |

TABLE W3A
Exemplary YYBYFW Self-replicating Rescue (SRR) plasmid

| | |
|---|---|
| Name | pRTx-4192 |
| Type | Plasmid |
| Description | pSRR::RING-YYBYFW |
| Length | 9274 bp |

(SEQ ID NO: 2774)
Annotations:

| Region/Element | Base range |
|---|---|
| EMCV internal ribosome entry site (IRES) | 81-667 |
| SV40 Large T Antigen coding sequence | 914-2794 |
| SV40 nuclear localization sequence | 1043-1063 |
| KT3 epitope tag | 2759-2791 |
| SV40 polyA sequence | 2825-2946 |
| SV40 polyA sequence (complement) | 2825-2946 |
| Origin of replication | 2986-3121 |
| LacO repressor binding site (complement) | 3201-3223 |
| Lac operon operator | 3203-3219 |
| Lac operon promoter (complement) | 3227-3257 |
| C-tag | 3235-3246 |
| E. coli catabolite activator protein | 3272-3293 |
| (complement) | |
| E. coli catabolite activator protein | 3272-3293 |
| Origin of replication (complement) | 3581-4169 |
| Kanamycin resistance (KanR) CDS | 4347-5156 |
| (complement) | |
| EF-1-alpha intron A | 5949-6887 |
| Origin of replication | 6973-6984 |
| ORF2 coding sequence | 6985-7266 |
| ORF1 coding sequence | 7137-9065 |
| Polyarginine tag | 7248-7262 |
| ORF3 coding sequence | 9095-9274 |

TABLE W4
Exemplary Ring161 Self-replicating Rescue (SRR) plasmid

| | |
|---|---|
| Name | pRTx-4185 |
| Type | Plasmid |
| Description | pSRR::RING161 |
| Length | 9330 bp |

(SEQ ID NO: 2639)
Annotations:

| Region/Element | Base range |
|---|---|
| EF-1-alpha intron A | 680-1618 |
| ORF2-3 coding sequence | 1669-1965 and 3662-4060 |
| ORF2-2 coding sequence | 1669-1965 and 3535-3819 |
| ORF2 coding sequence | 1669-1968 |
| ORF2 motif | 1723-1770 |
| ORF1 coding sequence | 1857-3839 |
| Polyarginine tag | 1881-1895 |
| Polyarginine tag | 1887-1901 |
| ORF1 motif | 2991-3023 |
| ORF3 coding sequence | 3641-4060 |
| ORF3 motif 2 | 3923-4000 |
| ORF3 motif 2 | 4031-4045 |
| EMCV internal ribosome entry site (IRES) | 4142-4728 |
| SV40 Large T Antigen coding sequence | 4975-6855 |
| SV40 nuclear localization sequence | 5104-5124 |
| KT3 epitope tag | 6820-6852 |
| SV40 polyA sequence | 6886-7007 |
| SV40 polyA sequence (complement) | 6886-7007 |
| Origin of replication | 7047-7182 |
| LacI repressor protein binding site (complement) | 7262-7284 |
| LacI repressor protein binding site | 7264-7280 |
| Lac operon operator | 7264-7280 |
| Lac operon promoter (complement) | 7288-7318 |
| C-tag | 7296-7307 |
| E. coli CAP binding site (complement) | 7333-7354 |
| E. coli CAP binding site | 7333-7354 |
| Origin of replication (complement) | 7642-8230 |
| Kanamycin resistance (KanR) CDS (complement) | 8408-9217 |

TABLE W5
Exemplary Ring10 Self-replicating Rescue (SRR) plasmid

| | |
|---|---|
| Name | pRTx-4177 |
| Type | Plasmid |
| Description | pSRR::RING10 |
| Length | 9473 bp |

(SEQ ID NO: 2640)
Annotations:

| Region/Element | Base range |
|---|---|
| EMCV internal ribosome entry site (IRES) | 81-667 |
| SV40 Large T Antigen coding sequence | 914-2794 |
| SV40 nuclear localization sequence | 1043-1063 |
| KT3 epitope tag | 2759-2791 |
| SV40 polyA sequence | 2825-2946 |
| SV40 polyA sequence (complement) | 2825-2946 |
| Origin of replication | 2986-3121 |
| LacI repressor protein binding site (complement) | 3201-3223 |
| LacI repressor protein binding site | 3203-3219 |
| Lac operon operator | 3203-3219 |
| Lac operon promoter (complement) | 3227-3257 |
| C-tag | 3235-3246 |
| E. coli CAP binding site (complement) | 3272-3293 |
| E. coli CAP binding site | 3272-3293 |
| Origin of replication (complement) | 3581-4169 |
| Kanamycin resistance (KanR) CDS (complement) | 4347-5156 |
| EF-1-alpha intron A | 5949-6887 |
| 5′UTR conserved domain | 6978-7048 |
| Origin of replication | 6978-6989 |
| ORF2 coding sequence | 7082-7372 |
| ORF1 coding sequence | 7261-9279 |
| Polyarginine tag | 7351-7365 |
| Factor Xa recognition and cleavage site | 8692-8703 |
| ORF3 coding sequence | 9117-9473 |
| ORF3 motif 2 | 9447-9461 |

TABLE W6
Exemplary Ring34 Self-replicating Rescue (SRR) plasmid

| | |
|---|---|
| Name | pRTx-3906 |
| Type | Plasmid |
| Description | pSRR::hEFla_Ring34 |
| Length | 9320 bp |

(SEQ ID NO: 2641)
Annotations:

| Region/Element | Base range |
|---|---|
| EF-1-alpha intron A | 669-1607 |
| ORF3 coding sequence | 1675-1959 and 3764-4060 |
| ORF2 coding sequence | 1675-1962 |
| ORF1 coding sequence | 1845-3842 |
| ORF3 coding sequence | 3668-4060 |
| EMCV internal ribosome entry site (IRES) | 4132-4718 |
| SV40 Large T Antigen coding sequence | 4965-6845 |
| SV40 Nuclear localization sequence | 5094-5114 |
| KT3 epitope tag | 6810-6842 |
| SV40 polyA sequence (complement) | 6876-6997 |
| SV40 polyA sequence | 6876-6997 |
| Origin of replication | 7037-7172 |
| LacI repressor protein binding site (complement) | 7252-7274 |
| Lac operon operator | 7254-7270 |
| Lac operon promoter (complement) | 7278-7308 |
| C-tag | 7286-7297 |
| E. coli CAP binding site (complement) | 7323-7344 |
| E. coli CAP binding site | 7323-7344 |
| Origin of replication (complement) | 7632-8220 |
| Kanamycin resistance (KanR) CDS (complement) | 8398-9207 |

TABLE W7
Exemplary Ring9 Self-replicating Rescue (SRR) plasmid

| | |
|---|---|
| Name | pRTx-3905 |
| Type | Plasmid |
| Description | pSRR::hEF1a_Ring9 |
| Length | 9289 bp |

(SEQ ID NO: 2642)
Annotations:

| Region/Element | Base range |
|---|---|
| EF-1-alpha intron A | 669-1607 |
| ORF2/3 coding sequence | 1677-1996, 3645-4029 |
| ORF2/2 coding sequence | 1677-1996, 3470-3806 |
| ORF2 coding sequence | 1677-2000 |
| ORF1 coding sequence | 1859-3826 |
| ORF3 coding sequence | 3625-4029 |
| EMCV internal ribosome entry site (IRES) | 4101-4687 |
| SV40 Large T Antigen coding sequence | 4934-6814 |
| SV40 nuclear localization sequence | 5063-5083 |
| KT3 epitope tag | 6779-6811 |
| SV40 polyA sequence (complement) | 6845-6966 |
| SV40 polyA sequence | 6845-6966 |
| Origin of replication | 7006-7141 |
| LacI repressor protein binding site (complement) | 7221-7243 |
| Lac operon operator | 7223-7239 |
| Lac operon promoter (complement) | 7247-7277 |
| C-tag | 7255-7266 |
| E. coli CAP binding site (complement) | 7292-7313 |
| E. coli CAP binding site | 7292-7313 |
| Origin of replication (complement) | 7601-8189 |
| Kanamycin resistance (KanR) CDS (complement) | 8367-9176 |

TABLE W8
Exemplary Ring25 Self-replicating Rescue (SRR) plasmid

| | |
|---|---|
| Name | pRTx-3861 |
| Type | Plasmid |
| Description | pSRR::hEF1a_Ring25 |
| Length | 8635 bp |

(SEQ ID NO: 2643)
Annotations:

| Region/Element | Base range |
|---|---|
| Origin of replication (complement) | 1-589 |
| Kanamycin resistance (KanR) CDS (complement) | 767-1576 |
| pHEf1A promoter | 1774-2947 |
| ORF2 coding sequence | 3116-3418 |
| ORF1 coding sequence | 3289-5271 |
| ORF3 coding sequence | 5229-5477 |
| 3′UTR | 5481-5504 |
| EMCV internal ribosome entry site (IRES) | 5509-6095 |
| SV40 Large T Antigen coding sequence | 6342-8222 |
| SV40 nuclear localization sequence | 6471-6491 |
| SV40 polyA sequence (complement) | 8253-8374 |
| SV40 polyA sequence | 8253-8374 |
| Origin of replication | 8414-8549 |

### Bacterial Minicircle Systems

Site-specific recombinases can be used to produce circular double-stranded genetic elements (e.g., minicircles) in host cells (e.g., bacterial host cells), which can then, in some embodiments, be isolated from the host cells and used for producing Anelloviridae family vectors via in vitro assembly. In some embodiments, a vector plasmid comprising the sequence of a genetic element flanked by recombinase recognition sites is present in a host cell (e.g., a bacterial host cell, e.g., an E. coli cell) along with a site-specific recombinase capable of recognizing the recombinase recognition sites and initiating their recombination. In certain embodiments, the site-specific recombinase is encoded in the bacterial genome. In other embodiments, the site-specific recombinase is encoded in a second plasmid (e.g., an expression plasmid as described herein). In further embodiments, the site-specific recombinase is encoded by the vector plasmid itself (e.g., in a cassette outside the region flanked by the recombinase recognition sites).

In some embodiments, the site-specific recombinase is PhiC31. In certain embodiments, the vector plasmid comprises a genetic element sequence flankedby an attB site and an attP site recognizable by PhiC31, e.g., as described herein.

Non-limiting examples of vector plasmids suitable for use in bacterial cells as described herein are provided in Tables Y1, Y3, and Y5 below. In some embodiments, the vector plasmid comprises the nucleic acid sequence of a vector plasmid as listed in any of Tables Y1, Y3, or Y5 (or an element or subsequence thereof as described in the respective table), or a nucleic acid sequence having at least 75%, 80%, 85%, 90%, 95%, 96%, 97%, 98%, or 99% identity thereto. In some embodiments, the minicircle comprises the nucleic acid sequence of a vector plasmid as listed in any of Tables Y2, Y4, or Y6 (or an element or subsequence thereof as described in the respective table), or a nucleic acid sequence having at least 75%, 80%, 85%, 90%, 95%, 96%, 97%, 98%, or 99% identity thereto.

In any of the embodiments, the coding sequence of the transgene (e.g., eGFP) in the vector plasmids may be replaced with any transgene, e.g., a transgene encoding an exogenous effector, e.g., a therapeutic effector (e.g., as described herein).

TABLE Y1
Exemplary Ring2 parental vector plasmid (pRTx-3928)

| | |
|---|---|
| Name | pRTx-3928 |
| Type | Plasmid |
| Description | Ring2 minicircle parental plasmid |
| Length | 6951 bp |

(SEQ ID NO: 2775)
Annotations:

| Region/Element | Base range |
|---|---|
| Origin of replication (complement) | 1-589 |
| Ampicillin resistance gene promoter | 660-704 |
| (complement) | |
| rrnB T2 terminator (complement) | 724-751 |
| rrnB T1 terminator (complement) | 843-929 |
| 5S ribosomal RNA (complement) | 931-1046 |
| tnpA fragment CDS | 1159-1431 |
| Neomycin/Kanamycin resistance CDS | 1463-2257 |
| Phage phi-C31 attP recognition site | 2344-2383 |
| PaqCI forward | 2390-2404 |
| bGHpA terminator sequence | 2416-2640 |
| WPRE | 2658-3246 |
| Factor Xa recognition and cleavage site | 2764-2775 |
| eGFP CDS (complement) | 3260-3976 |
| Kozak sequence | 3973-3982 |
| Full CMV promoter | 4030-4599 |
| CMV promoter | 4031-4234 |
| CMV enhancer | 4235-4599 |
| CMV enhancer | 4235-4538 |
| Ring2 NCR | 4611-5200 |
| Intron 1 | 4611-4699 |
| 5′UTR conserved domain | 4641-4711 |
| Origin of replication | 4641-4711 |
| Initiator element | 4762-4777 |
| Promoter | 4769-5012 |
| GC-rich region | 5034-5145 |
| VL46-002-LY2 gene (complement) | 5036-5200 |
| PaqCI forward | 5201-5215 |
| Phage phi-C31 attB site | 5216-5220 |
| Phage phi-C31 attB site (complement) | 5221-5249 |
| araC CDS | 6072-6362 |
| Rop (repressor of primer) CDS | 6484-6524 |
| Bom (basis of mobility) site | 6626-6766 |

Exemplary tnpA Transposase Fragment Amino Acid Sequence (e.g., Encoded by Nucleotides 1159-1431 of Table Y1):

(SEQ ID NO: 2644)
ERVESQSAETVLTPDECQLLGYLDKGKRKRKEKAGSLQWAYMAIARLGG
FMDSKRTGIASWGALW*GWEALQSKLDGFLAAKDLMAQGIKL

### Exemplary Neomycin/Kanamycin Resistance Amino Acid Sequence (e.g., Encoded by Nucleotides 1463-2257 of Table Y1):

(SEQ ID NO: 2645)
MIEQDGLHAGSPAAWVERLFGYDWAQQTIGCSDAAVFRLSAQGRPVLFV
KTDLSGALNELQDEAARLSWLATTGVPCAAVLDVVTEAGRDWLLLGEVP
GQDLLSSHLAPAEKVSIMADAMRRLHTLDPATCPFDHQAKHRIERARTR
MEAGLVDQDDLDEEHQGLAPAELFARLKASMPDGEDLVVTHGDACLPNI
MVENGRFSGFIDCGRLGVADRYQDIALATRDIAEELGGEWADRFLVLYG
IAAPDSQRIAFYRLLDEFF

### Exemplary Factor Xa Recognition and Cleavage Site Amino Acid Sequence (e.g., Encoded by Nucleotides 2764-2775 of Table Y1):

(SEQ ID NO: 2646)
IEGR

Exemplary araC Fragment Amino Acid Sequence (e.g., Encoded by Nucleotides 6072-6362 of Table Y1):

(SEQ ID NO: 2647)
IASVAQHVCLSPSRLSHLFRQQLGISVLSWREDQRISQAKLLLSTTRMP
IATVGRNVGFDDQLYFSRVFKKCTGASPSEFRAGCEEKVNDVAVKLS

### Exemplary Rop Fragment Amino Acid Sequence (e.g., Encoded by Nucleotides 6484-6524 of Table Y1):

(SEQ ID NO: 2648)
AASRVSVMTVKTS

TABLE Y2
Exemplary Ring2 minicircle post-recombination
by PhiC31 (pRTx-3928_MC)

| | |
|---|---|
| Name | pRTx-3928_MC |
| Type | Circular DNA |
| Description | Ring2 minicircle post recombination |
| Length | 2869 bp |

(SEQ ID NO: 2649)
Annotations:

| Region/Element | Base range |
|---|---|
| attB site | 1-10 |
| Synthesize | 11-624 |
| attB site | 11-15 |
| PaqCI forward | 16-30 |
| Ring2 NCR | 31-620 |
| VL46-002-LY2 gene | 31-195 |
| GC-rich region | 86-197 |
| Promoter | 219-462 |
| Initiator element | 454-469 |
| 5′UTR conserved domain | 520-590 |
| Origin of replication | 520-590 |
| Intron 1 | 532-620 |
| Full CMV promoter | 632-1201 |
| CMV enhancer | 632-996 |
| CMV enhancer | 693-996 |
| CMV promoter | 997-1200 |
| Kozak sequence | 1249-1258 |
| eGFP | 1255-1971 |
| WPRE | 1985-2573 |
| Factor Xa recognition and cleavage site (complement) | 2456-2467 |
| bGHpA terminator sequence | 2591-2815 |
| PaqCI forward | 2827-2841 |
| attP site fragment | 2848-2869 |

Exemplary eGFP Amino Acid Sequence (e.g., Encoded by Nucleotides 1255-1971 of Table Y2):

(SEQ ID NO: 2650)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

TABLE Y3
Exemplary Ring19 parental vector plasmid (pRTx-3929)

| | |
|---|---|
| Name | pRTx-3929 |
| Type | Plasmid |
| Description | Ring19 minicircle parental plasmid |
| Length | 6879 bp |

(SEQ ID NO: 2651)
Annotations:

| Region/Element | Base range |
|---|---|
| Origin of replication (complement) | 1-589 |
| Ampicillin resistance promoter (complement) | 660-704 |
| rrnB T2 terminator (complement) | 724-751 |
| rrnB T1 terminator (complement) | 843-929 |
| 5S ribosomal RNA (complement) | 931-1046 |
| tnpA fragment CDS | 1159-1431 |
| Neomycin/Kanamycin resistance gene CDS | 1463-2257 |
| Phage phi-C31 attP site | 2344-2383 |
| PaqCI forward | 2390-2404 |
| bGHpA terminator sequence | 2416-2640 |
| WPRE | 2658-3246 |
| Factor Xa recognition and cleavage site | 2764-2775 |
| eGFP CDS (complement) | 3260-3976 |
| Kozak sequence | 3973-3982 |
| Full CMV promoter | 4030-4599 |
| CMV promoter | 4031-4234 |
| CMV enhancer | 4235-4599 |
| CMV enhancer | 4235-4538 |
| Ring 19 NCR | 4611-5128 |
| Intron 1 | 4611-4698 |
| 5′UTR conserved domain | 4640-4710 |
| Origin of replication | 4640-4710 |
| Initiator element | 4762-4777 |
| GC-rich region | 4950-5061 |
| PaqCI forward | 5129-5143 |
| Phage phi-C31 attB site | 5144-5148 |
| Phage phi-C31 attB site (complement) | 5149-5177 |
| araC fragment CDS | 6000-6290 |
| rop fragment CDS | 6412-6452 |
| bom | 6554-6694 |

TABLE Y4
Exemplary Ring19 minicircle post-recombination
by PhiC31 (pRTx-3929_MC)

| | |
|---|---|
| Name | pRTx-3929_MC |
| Type | Circular DNA |
| Description | Ring19 minicircle post recombination |
| Length | 2797 bp |

(SEQ ID NO: 2652)
Annotations:

| Region/Element | Base range |
|---|---|
| attB site | 1-10 |
| Synthesize | 11-552 |
| attB site | 11-15 |
| PaqCI forward | 16-30 |
| Ring19 NCR | 31-548 |
| GC-rich region | 98-209 |
| Initiator element | 382-397 |
| 5′UTR conserved domain | 449-519 |
| Origin of replication | 449-519 |
| Intron 1 | 461-548 |
| Full CMV promoter | 560-1129 |
| CMV enhancer | 560-924 |
| CMV enhancer | 621-924 |
| CMV promoter | 925-1128 |
| Kozak sequence | 1177-1186 |
| eGFP CDS | 1183-1899 |
| WPRE | 1913-2501 |
| Factor Xa recognition and cleavage site | 2384-2395 |
| (complement) | |
| bGHpA terminator sequence | 2519-2743 |
| PaqCI forward | 2755-2769 |
| attP site fragment | 2776-2797 |

TABLE Y5
Exemplary Ring20 parental vector plasmid (pRTx-3930)

| | |
|---|---|
| Name | pRTx-3930 |
| Type | Plasmid |
| Description | Ring20 minicircle parental plasmid |
| Length | 7378 bp |

(SEQ ID NO: 2653)
Annotations:

| Region/Element | Base range |
|---|---|
| Origin of replication (complement) | 1-589 |
| Ampicillin resistance gene promoter | |
| (complement) | 660-704 |
| rrnB T2 terminator (complement) | 724-751 |
| rrnB T1 terminator (complement) | 843-929 |
| 5S ribosomal RNA (complement) | 931-1046 |
| tnpA fragment CDS | 1159-1431 |
| Neomycin/kanamycin resistance gene CDS | 1463-2257 |
| attP site | 2344-2383 |
| PaqCI forward | 2390-2404 |
| bGHpA terminator sequence | 2416-2640 |
| WPRE | 2658-3246 |
| Factor Xa recognition and cleavage site CDS | 2764-2775 |
| eGFP CDS (complement) | 3260-3976 |
| Kozak sequence | 3973-3982 |
| Full CMV promoter | 4030-4599 |
| CMV promoter | 4031-4234 |
| CMV enhancer | 4235-4599 |
| CMV enhancer | 4235-4538 |
| Ring20 NCR | 4611-5627 |
| GC-rich region | 5489-5589 |
| PaqCI forward | 5628-5642 |
| attB site | 5643-5647 |
| attB site (complement) | 5648-5676 |
| araC fragment CDS | 6499-6789 |
| Rop fragment CDS | 6911-6951 |
| Bom | 7053-7193 |

TABLE Y6
Exemplary Ring20 minicircle post-recombination
by PhiC31 (pRTx-3930_MC)

| | |
|---|---|
| Name | pRTx-3930_MC |
| Type | Circular DNA |
| Description | Ring20 minicircle post recombination |
| Length | 3296 bp |

(SEQ ID NO: 2654)
Annotations:

| Region/Element | Base range |
|---|---|
| attB site | 1-10 |
| attB site | 11-15 |
| PaqCI forward | 16-30 |
| Ring20 NCR | 31-1047 |
| GC-rich region | 69-169 |
| Full CMV promoter | 1059-1628 |
| CMV enhancer | 1059-1423 |
| CMV enhancer | 1120-1423 |
| CMV promoter | 1424-1627 |
| Kozak sequence | 1676-1685 |
| eGFP CDS | 1682-2398 |
| WPRE | 2412-3000 |
| Factor Xa recognition and cleavage site | 2883-2894 |
| (complement) | |
| bGHpA terminator sequence | 3018-3242 |
| PaqCI forward | 3254-3268 |
| attP site fragment | 3275-3296 |

Conversion of Minicircles to cssDNA Genetic Elements

In some embodiments, circular double-stranded DNA molecules (e.g., minicircles) comprising the sequence of a genetic element (e.g., produced by a site-specific recombinase system as described herein) are converted to circular single-stranded DNA (cssDNA) molecules that can serve as genetic elements for Anelloviridae family vectors. In some embodiments, the circular double-stranded DNA molecule is contacted with a nicking enzyme that selectively introduces nicks into one strand of the DNA molecule and not the other strand. In some embodiments, at least 1 U of the nicking enzyme per μg of DNA is used. In some embodiments, the circular double-stranded DNA molecule is incubated with the nicking enzyme at 37° C. for 2.5 hours. The nicked double-stranded DNA molecule is then digested with an exonuclease that selectively digests nicked DNA strands, resulting in the degradation of the nicked strand and leaving the un-nicked DNA strand behind as a single-stranded DNA molecule. In some embodiments, at least 1 μl exonuclease per μg DNA is used. In some embodiments, the nicked double-stranded DNA molecule is incubated with the exonuclease at 25° C. for 30 minutes. In some embodiments, the exonuclease reaction is stopped by adding EDTA to 11 mM.

In some embodiments, the cssDNA molecule is isolated by phenol:chloroform extraction and ethanol precipitation. In some embodiments, one volume of a phenol:chloroform solution is added to the exonuclease-digested mixture. In some embodiments, the aqueous phase is aspirated from the top of the solution (e.g., the nucleic acid sample). In some embodiments, ₁/1₀th volume of 3M sodium acetate is added to the nucleic acid sample followed by 1 volume of 99% isopropanol and centrifugation for 30 minutes at 4° C. and 12,000×g. In some embodiments, the pellet is rinsed, air dried, and then dissolved in water.

The resulting product is a circular single-stranded DNA (cssDNA) molecule, which is the same structure as that of the viral genomes for Anelloviridae family viruses. Such cssDNA molecules can thus be genetic elements that are packaged into Anelloviridae family vector particles, e.g., by being encapsulated within a proteinaceous exterior comprising Anellovirus ORF1 molecules or CAV VP1 molecules (e.g., as described herein). In certain embodiments, the cssDNA is packaged into an Anelloviridae family vector particle via in vitro assembly, e.g., as described herein. In other embodiments, the cssDNA is introduced (e.g., transfected) into a host cell (e.g., a mammalian host cell) capable of expressing an Anellovirus ORF1 molecule or CAV VP1 molecule, and is packaged into an Anelloviridae family vector particle in the host cell.

In some embodiments, the sense strand of a circular double-stranded DNA molecule (e.g., minicircle) is digested and the antisense strand is left behind as a cssDNA. In some embodiments, the antisense strand of a circular double-stranded DNA molecule (e.g., minicircle) is digested and the sense strand is left behind as a cssDNA. In some embodiments, the nicking enzyme is an Nt.BsmAI enzyme (e.g., as described herein). In some embodiments, the nicking enzyme is Nt.BbvCI. In some embodiments, the nicking enzyme is Nb.BsrDI. In some embodiments, the nicking enzyme is Nb.BssSI. In some embodiments, the nicking enzyme is nicking Cas9. In some embodiments, the exonuclease is a T7 exonuclease (e.g., as described herein). In some embodiments, the exonuclease is λexo. In some embodiments, the exonuclease is ExoII. In some embodiments, the exonuclease is Exo VIII.

### Exemplary Site-Specific Recombinases and Recombinase Recognition Sites

The recombinase-based systems described herein utilize site-specific recombinases to induce recombination of vector plasmids at recombinase recognition sites, thereby producing double-stranded DNA molecules (e.g., minicircles) comprising the sequence between the recombinase recognition sites (e.g., comprising the sequence of a genetic element for an Anelloviridae family vector as described herein). In some embodiments, the site-specific recombinase is a serine recombinase. In some embodiments, the site-specific recombinase is a tyrosine recombinase. In some embodiments, the site-specific recombinase comprises a nuclear localization signal (NLS), e.g., an SV40 NLS. In some embodiments, the site-specific recombinase comprises an epitope tag (e.g., an HA tag, e.g., comprising the amino acid sequence YPYDVPDYA (SEQ ID NO: 2504).

In some embodiments, the site-specific recombinase comprises the amino acid sequence of a recombinase as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of SV40-NLS-iCre as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of SV40-NLS-HA_Bxb1 as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of Cre as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of Bxb1 as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of PhiC31 as listed in Table V2, or an amino acid sequence having at least 75%, 80%, 85%, 90%, 95%, 96%, 97%, 98%, or 99% identity thereto. In some embodiments, the site-specific recombinase comprises the amino acid sequence of ParA as listed in Table V2, or an amino acid sequence having at least 75%, 80%, 85%, 90%, 95%, 96%, 97%, 98%, or 99% identity thereto.

In some embodiments, the site-specific recombinase comprises a Cre recombinase (e.g., as described herein, e.g., in Table V2), or an amino acid sequence having at least 75%, 80%, 85%, 90%, 95%, 96%, 97%, 98%, or 99% identity thereto. In certain embodiments, at least one (e.g., one or both) of the recombinase recognition sites comprises a lox66 site as listed in Table V3, or a nucleic acid sequence having at least 75%, 80%, 85%, 90%, 95%, 96%, 97%, 98%, or 99% identity thereto. In certain embodiments, at least one (e.g., one or both) of the recombinase recognition sites comprises a lox71 site as listed in Table V3, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Cre recombination event (e.g., as described herein) comprises a loxP site as listed in Table V3, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Cre recombination event (e.g., as described herein) comprises a lox72 site as listed in Table V3, or a nucleic acid sequence having at least 75%, 80%, 85%, 90%, 95%, 96%, 97%, 98%, or 99% identity thereto.

In some embodiments, the site-specific recombinase comprises a Bxb1 recombinase (e.g., as described herein, e.g., in Table V2), or an amino acid sequence having at least 75%, 80%, 85%, 90%, 95%, 96%, 97%, 98%, or 99% identity thereto. In certain embodiments, at least one of the recombinase recognition sites comprises an attB site as listed in Table V4, or a nucleic acid sequence having at least 75%, 80%, 85%, 90%, 95%, 96%, 97%, 98%, or 99% identity thereto. In certain embodiments, at least one of the recombinase recognition sites comprises an attP site as listed in Table V4, or a nucleic acid sequence having at least 75%, 80%, 85%, 90%, 95%, 96%, 97%, 98%, or 99% identity thereto. In certain embodiments, one recombinase recognition site comprises an attB site as listed in Table V4, or a nucleic acid sequence having at least 75%, 80%, 85%, 90%, 95%, 96%, 97%, 98%, or 99% identity thereto, and the other recombinase recognition site comprises an attP site as listed in Table V4, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Bxb1 recombination event (e.g., as described herein) comprises an attL site as listed in Table V4, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a Bxb1 recombination event (e.g., as described herein) comprises an attR site as listed in Table V4, or a nucleic acid sequence having at least 75%, 80%, 85%, 90%, 95%, 96%, 97%, 98%, or 99% identity thereto.

In some embodiments, the site-specific recombinase comprises a PhiC31 recombinase (e.g., as described herein, e.g., in Table V2), or an amino acid sequence having at least 75%, 80%, 85%, 90%, 95%, 96%, 97%, 98%, or 99% identity thereto. In certain embodiments, at least one of the recombinase recognition sites comprises an attB site as listed in Table V5, or a nucleic acid sequence having at least 75%, 80%, 85%, 90%, 95%, 96%, 97%, 98%, or 99% identity thereto. In certain embodiments, at least one of the recombinase recognition sites comprises an attP site as listed in Table V5, or a nucleic acid sequence having at least 75%, 80%, 85%, 90%, 95%, 96%, 97%, 98%, or 99% identity thereto. In certain embodiments, one recombinase recognition site comprises an attB site as listed in Table V5, or a nucleic acid sequence having at least 75%, 80%, 85%, 90%, 95%, 96%, 97%, 98%, or 99% identity thereto, and the other recombinase recognition site comprises an attP site as listed in Table V5, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a PhiC31 recombination event (e.g., as described herein) comprises an attL site as listed in Table V5, or a nucleic acid sequence having at least 75%, 80%, 85%, 90%, 95%, 96%, 97%, 98%, or 99% identity thereto. In certain embodiments, a circular double-stranded nucleic acid molecule (e.g., minicircle) produced after a PhiC31 recombination event (e.g., as described herein) comprises a an attR site as listed in Table V5, or a nucleic acid sequence having at least 75%, 80%, 85%, 90%, 95%, 96%, 97%, 98%, or 99% identity thereto.

In some embodiments, the site-specific recombinase comprises a ParA resolvase (e.g., as described herein, e.g., in Table V2), or an amino acid sequence having at least 75%, 80%, 85%, 90%, 95%, 96%, 97%, 98%, or 99% identity thereto. In certain embodiments, at least one (e.g., one or both) of the recombinase recognition sites comprises an MRS sequence (e.g., the sequence of GGTCAAATTGGGTATACCCATTTGGGCCTAGTCTAGCCGGCATGGCGCATTACAGCAATACGCAATT TAAATGCGCCTAGCGCATTTTCCCGACCTTAATGCGCCTCGCGCTGTAGCCTCACGCCCACATATG; SEQ ID NO: 810), or a nucleic acid sequence having at least 75%, 80%, 85%, 90%, 95%, 96%, 97%, 98%, or 99% identity thereto. In certain embodiments, both of the recombinase recognition sites comprise an MRS sequence (e.g., the nucleic acid sequence of SEQ ID NO: 810), or a nucleic acid sequence having at least 75%, 80%, 85%, 90%, 95%, 96%, 97%, 98%, or 99% identity thereto. In certain embodiments, the recombinase recognition sites have the same sequence (e.g., an MRS sequence, e.g., comprising the nucleic acid sequence of SEQ ID NO: 810, or a nucleic acid sequence having at least 75%, 80%, 85%, 90%, 95%, 96%, 97%, 98%, or 99% identity thereto).

TABLE V2
Exemplary site-specific recombinase polypeptides

| Site-specific | Amino Acid Sequence |
|---|---|
| recombinase | (Underline = SV40 NLS sequence; bolded italics = HA tag sequence) |
| SV40- | MVPKKKRKVSNLLTVHQNLPALPVDATSDEVRKNLMDMFRDRQAFSEHTWKMLLSVCRS |
| NLS_iCre | WAAWCKLNNRKWFPAEPEDVRDYLLYLQARGLAVKTIQQHLGQLNMLHRRSGLPRPSDS |
| | NAVSLVMRRIRKENVDAGERAKQALAFERTDFDQVRSLMENSDRCQDIRNLAFLGIAYN |
| | TLLRIAEIARIRVKDISRTDGGRMLIHIGRTKTLVSTAGVEKALSLGVTKLVERWISVS |
| | GVADDPNNYLFCRVRKNGVAAPSATSQLSTRALEGIFEATHRLIYGAKDDSGQRYLAWS |
| | GHSARVGAARDMARAGVSIPEIMQAGGWTNVNIVMNYIRNLDSETGAMVRLLEDGD* |
| | (SEQ ID NO: 2655) |
| SV40-NLS- | MPKKKRKVYPYDVPDYAGSRALVVIRLSRVTDATTSPERQLESCQQLCAQRGWDVVGVA |
| HA_Bxb1 | EDLDVSGAVDPFDRKRRPNLARWLAFEEQPFDVIVAYRVDRLTRSIRHLQQLVHWAEDH |
| | KKLVVSATEAHFDTTTPFAAVVIALMGTVAQMELEAIKERNRSAAHFNIRAGKYRGSLP |
| | PWGYLPTRVDGEWRLVPDPVQRERILEVYHRVVDNHEPLHLVAHDLNRRGVLSPKDYFA |
| | QLQGREPQGREWSATALKRSMISEAMLGYATLNGKTVRDDDGAPLVRAEPILTREQLEA |
| | LRAELVKTSRAKPAVSTPSLLLRVLFCAVCGEPAYKFAGGGRKHPRYRCRSMGFPKHCG |
| | NGTVAMAEWDAFCEEQVLDLLGDAERLEKVWVAGSDSAVELAEVNAELVDLTSLIGSPA |
| | YRAGSPQREALDARIAALAARQEELEGLEARPSGWEWRETGQRFGDWWREQDTAAKNTW |
| | LRSMNVRLTFDVRGGLTRTIDFGDLQEYEQHLRLGSVVERLHTGMS* (SEQ ID NO: |
| | 2656) |
| Cre (same as | MSNLLTVHQNLPALPVDATSDEVRKNLMDMFRDRQAFSEHTWKMLLSVCRSWAAWCKLN |
| iCre) | NRKWFPAEPEDVRDYLLYLQARGLAVKTIQQHLGQLNMLHRRSGLPRPSDSNAVSLVMR |
| | RIRKENVDAGERAKQALAFERTDFDQVRSLMENSDRCQDIRNLAFLGIAYNTLLRIAEI |
| | ARIRVKDISRTDGGRMLIHIGRTKTLVSTAGVEKALSLGVTKLVERWISVSGVADDPNN |
| | YLFCRVRKNGVAAPSATSQLSTRALEGIFEATHRLIYGAKDDSGQRYLAWSGHSARVGA |
| | ARDMARAGVSIPEIMQAGGWTNVNIVMNYIRNLDSETGAMVRLLEDGD (SEQ ID NO: |
| | 2657) |
| Bxb1 | MRALVVIRLSRVTDATTSPERQLESCQQLCAQRGWDVVGVAEDLDVSGAVDPFDRKRRP |
| | NLARWLAFEEQPFDVIVAYRVDRLTRSIRHLQQLVHWAEDHKKLVVSATEAHFDTTTPF |
| | AAVVIALMGTVAQMELEAIKERNRSAAHFNIRAGKYRGSLPPWGYLPTRVDGEWRLVPD |
| | PVQRERILEVYHRVVDNHEPLHLVAHDLNRRGVLSPKDYFAQLQGREPQGREWSATALK |
| | RSMISEAMLGYATLNGKTVRDDDGAPLVRAEPILTREQLEALRAELVKTSRAKPAVSTP |
| | SLLLRVLFCAVCGEPAYKFAGGGRKHPRYRCRSMGFPKHCGNGTVAMAEWDAFCEEQVL |
| | DLLGDAERLEKVWVAGSDSAVELAEVNAELVDLTSLIGSPAYRAGSPQREALDARIAAL |
| | AARQEELEGLEARPSGWEWRETGQRFGDWWREQDTAAKNTWLRSMNVRLTFDVRGGLTR |
| | TIDFGDLQEYEQHLRLGSVVERLHTGMS (SEQ ID NO: 2658) |
| PhiC31 | MDTYAGAYDRQSRERENSSAASPATQRSANEDKAADLQREVERDGGRFRFVGHFSEAPG |
| | TSAFGTAERPEFERILNECRAGRLNMIIVYDVSRFSRLKVMDAIPIVSELLALGVTIVS |
| | TQEGVFRQGNVMDLIHLIMRLDASHKESSLKSAKILDTKNLQRELGGYVGGKAPYGFEL |
| | VSETKEITRNGRMVNVVINKLAHSTTPLTGPFEFEPDVIRWWWREIKTHKHLPFKPGSQ |
| | AAIHPGSITGLCKRMDADAVPTRGETIGKKTASSAWDPATVMRILRDPRIAGFAAEVIY |
| | KKKPDGTPTTKIEGYRIQRDPITLRPVELDCGPIIEPAEWYELQAWLDGRGRGKGLSRG |
| | QAILSAMDKLYCECGAVMTSKRGEESIKDSYRCRRRKVVDPSAPGQHEGTCNVSMAALD |
| | KFVAERIFNKIRHAEGDEETLALLWEAARRFGKLTEAPEKSGERANLVAERADALNALE |
| | ELYEDRAAGAYDGPVGRKHFRKQQAALTLRQQGAEERLAELEAAEAPKLPLDQWFPEDA |
| | DADPTGPKSWWGRASVDDKRVFVGLFVDKIVVTKSTTGRGQGTPIEKRASITWAKPPTD |
| | DDEDDAQDGTEDVAA (SEQ ID NO: 2659) |
| ParA | MATREQQPEGRRLKVARIYLRASTDEQNLERQESLVAATRAAGYYVAGIYREKASGARA |
| | DRPELLRMIADLQPGEVVVAEKIDRISRLPLAEAERLVASIRAKGAKLAVPGVVDLSEL |
| | AAEANGVAKIVLESVQDMLLKLALQMARDDYEDRRERQRQGVQLAKAAGRYTGRKRDAG |
| | MHDRIITLRSGGSSIAKTAKLVGCSPSQVKRVWAAWNAQQQK (SEQ ID NO: 2660) |

TABLE V3
Exemplary recombinase recognition sequences and recombinase hybrid sites for Cre
recombinases

| Recombinase | |
|---|---|
| Recognition Site Name | Nucleic acid sequence |
| loxP | ATAACTTCGTATAGCATACATTATACGAAGTTAT (SEQ ID |
| | NO: 2661) |
| lox66 | ATAACTTCGTATAGCATACATTATACGAACGGTA (SEQ ID |
| | NO: 2662) |
| lox71 | TACCGTTCGTATAGCATACATTATACGAAGTTAT (SEQ ID |
| | NO: 2663) |
| lox72 (hybrid) | TACCGTTCGTATAGCATACATTATACGAACGGTA (SEQ ID |
| | NO: 2664) |

TABLE V4
Exemplary recombinase recognition sequences for Bxb1 recombinases

| Recombinase | |
|---|---|
| Recognition | |
| Site Name | Nucleic acid sequence |
| attB | TCGGCCGGCTTGTCGACGACGGCGGTCTCCGTCGTCAGGATCATCCGGGC (SEQ ID |
| | NO: 2665) |
| attP | GTCGTGGTTTGTCTGGTCAACCACCGCGGTCTCAGTGGTGTACGGTACAAACCCCGAC |
| | (SEQ ID NO: 2666) |
| attL | TCGGCCGGCTTGTCGACGACGGCGGTCTCAGTGGTGTACGGTACAAACCCCGAC (SEQ |
| | ID NO: 2667) |
| attR | GTCGTGGTTTGTCTGGTCAACCACCGCGGTCTCCGTCGTCAGGATCATCCGGGC (SEQ |
| | ID NO: 2668) |

TABLE V5
Exemplary recombinase recognition sequences for PhiC31 recombinases

| Recombinase | |
|---|---|
| Recognition | |
| Site Name | Nucleic acid sequence |
| attB | TGCGGGTGCCAGGGCGTGCCCTTGGGCTCCCCGGGCGCGTACTCC (SEQ ID NO: |
| | 2669) |
| attP | GTGCCCCAACTGGGGTAACCTTTGAGTTCTCTCAGTTGGGGG (SEQ ID NO: 2670) |
| attL | TGCGGGTGCCAGGGCGTGCCCTTGAGTTCTCTCAGTTGGGGG (SEQ ID NO: 2671) |
| attR | TTGCCCCAACTGGGGTAACCTTTGGGCTCCCCGGGCGCGTACTCC (SEQ ID NO: 2672) |

In some embodiments, the site-specific recombinase comprises a polypeptide as listed in Table VR below. In certain embodiments, the recombinase recognition sites flanking the genetic element sequence comprise sequences recognized by the site-specific recombinase as listed in Table V1. In some embodiments, the site-specific recombinase is a ParA resolvase (e.g., as described herein). In some embodiments, the site-specific recombinase is an A118 recombinase. In some embodiments, the site-specific recombinase is a ΦFC1 recombinase. In some embodiments, the site-specific recombinase comprises a polypeptide as described in Turan et al. (2011, FASEB J. 25: 4088-4107; incorporated herein by reference in its entirety). In certain embodiments, the recombinase recognition sites flanking the genetic element sequence comprise sequences recognized by the site-specific recombinase as described in Turan et al., supra.

TABLE V1
Exemplary site-specific recombinases and their
cognate recombinase recognition sites

| | | | Target recognition |
|---|---|---|---|
| | | | sites |
| | Recombinase | | (spacer/crossover |
| Recombinase | Class | Source Species | [bp]) |
| Flp | Tyrosine | 2 μ (Saccharomyces | FRT (8/8) |
| | Recombinase | cerevisiae) | |
| R | Tyrosine | pSR1 (Zygosaccharomyces | RS (7/7) |
| | Recombinase | rouxii) | |
| Cre | Tyrosine | P1 phage (E. coli) | loxP (8/6) |
| | Recombinase | | |
| Dre, VCre | Tyrosine | Cre-variant(s) | rox, (4/4) VloxP, etc. |
| | Recombinase | | |
| λ | Tyrosine | λ phage (E. coli) | attP/attB → attL/attR |
| | Recombinase | | |
| HK022 | Tyrosine | HK022 phage (E. coli) | attP/attB → attL/attR |
| | Recombinase | | |
| HP1 | Tyrosine | Phage HP1 (Haemophilus) | attP/attB → attL/attR |
| | Recombinase | | |
| XerC/D | Tyrosine | E. coli | attP/attB → attL/attR |
| | Recombinase | | |
| γδ- res. | Serine | Tn1000 (E. coli) | attP/attB → attL/attR |
| | Recombinase | | |
| ParA resolvase | Serine | RK2/RP4 bact. plasmids | attP/attB → attL/attR |
| | Recombinase | (gram-) | |
| Tn3 | Serine | Tn3 transposon (E. coli) | attP/attB → attL/attR |
| | Recombinase | | |
| Gin | Serine | Salmonella | attP/attB → attL/attR |
| | Recombinase | | |
| ΦC31 | Serine | Φ31 phage (Streptomyces | attP/attB → attL/attR |
| | Recombinase | lividans) | |
| ΦBT1 | Serine | ΦBT1 phage | attP/attB → attL/attR |
| | Recombinase | (Streptomyces) | |
| Bxb1 | Serine | Bxb1 phage (Mycobact. | attP/attB → attL/attR |
| | Recombinase | smegmatis) | |
| R4 | Serine | Actinophage | attP/attB → attL/attR |
| | Recombinase | (Streptomyces parvulus) | |
| TP901-1 | Serine | TP901-1 phage | attP/attB → attL/attR |
| | Recombinase | (Lactococcus lactis) | |

### Genetic Element Constructs, e.g., for Assembly of Anellovectors

The genetic element of an anellovector as described herein may be produced from a genetic element construct that comprises a genetic element region and optionally other sequence such as vector backbone. Generally, the genetic element construct comprises an Anellovirus 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the anellovector as described herein (e.g., in a cell culture system), which may comprise one or more of the following steps: (a) introducing (e.g., transfecting) a genetic element (e.g., linearized) into a cell line sensitive to anellovector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

### Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

### Constructs Comprising Recombinase Recognition Sequences

In some embodiments, a genetic element construct (e.g., a plasmid) comprises a set of recombinase recognition sequences flanking the sequence of a genetic element of an Anelloviridae family vector as described herein. The recombinase recognition sites may be recognized by a site-specific recombinase as described herein. The remainder of the genetic element construct may, in some instances, comprise a vector backbone comprising elements for replication of the construct in a cell, such as a mammalian cell or a bacterial cell. Non-limiting examples of genetic element constructs comprising recombination recognition sites are provided in Tables Z1-Z18 and Y1-Y6 herein.

In some embodiments, the site-specific recombinase initiates a recombination event in the genetic element construct that separates the sequence of the genetic element from the remainder of the genetic element construct (e.g., the vector backbone). The recombination event may, in some embodiments, produce a small circular nucleic acid molecule (e.g., a minicircle) comprising the sequence of the genetic element. The minicircle may retain a scar at the site of recombination. In certain embodiments, the scar comprises a recombinase recognition sequence for the site-specific recombinase (e.g., a recombinase recognition sequence matching one of the recombinase recognition sequences in the original genetic element construct, or recombinase recognition sequence different from the recombinase recognition sequences in the original genetic element construct). In some embodiments, the remainder of the genetic element construct is also formed into a circular nucleic acid molecule after the recombination event (e.g., an empty vector backbone).

In some embodiments, the portion of the genetic element construct flanked by recombination recognition sites comprises, in order: (i) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a nucleic acid sequence encoding a transgene of interest; and (iii) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto.

In some embodiments, the portion of the genetic element construct flanked by recombination recognition sites comprises, in order: (i) a 3′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; (ii) a 5′ NCR of an Anelloviridae family virus (e.g., Ring2, Ring19, Ring20, or CAV), or a nucleic acid sequence having at least 75%, 80%, 85%, 90%, 95%, 96%, 97%, 98%, or 99% identity thereto; and (iii) a nucleic acid sequence encoding a transgene of interest.

### Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element construct is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

### Cis/Trans Constructs

In some embodiments, a genetic element construct as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell). In some embodiments, an ORF1 molecule is provided in trans, e.g., as described herein. In some embodiments, an ORF2 molecule is provided in trans, e.g., as described herein. In some embodiments, an ORF1 molecule and an ORF1 molecule are both provided in trans, e.g., as described herein.

In some embodiments, the genetic element construct comprises a sequence encoding an Anellovirus ORF1 molecule, or a splice variant or functional fragment thereof (e.g., a structural jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a structural jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a structural jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

### Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

### Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Effectors

The compositions and methods described herein can be used to produce a genetic element of an anellovector comprising a sequence encoding an effector (e.g., an exogenous effector or an endogenous effector), e.g., as described herein. The compositions and methods described herein can also be used to produce an anelloVLP comprising an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

In some embodiments, the effector comprises an intracellular agent (e.g., an intracellular polypeptide or intracellular nucleic acid), e.g., as described in PCT Publication No. WO 2020/123753 (incorporated herein by reference in its entirety.

In some embodiments, the effector comprises a secreted agent (e.g., a secreted polypeptide), e.g., as described in PCT Publication No. WO 2020/123773 (incorporated herein by reference in its entirety.

In some embodiments, the effector comprises a polypeptide that, when mutated, causes a disease or disorder, or a function variant of the polypeptide, e.g., as described in PCT Publication No. WO 2020/123795 (incorporated herein by reference in its entirety.

### Host Cells

The anellovectors described herein can be produced, for example, in a host cell. Generally, a host cell is provided that comprises an anellovector genetic element and the components of an anellovector proteinaceous exterior (e.g., a polypeptide encoded by an Anellovirus ORF1 nucleic acid or an Anellovirus ORF1 molecule). The host cell is then incubated under conditions suitable for enclosure of the genetic element within the proteinaceous exterior (e.g., culture conditions as described herein). In some embodiments, the host cell is further incubated under conditions suitable for release of the Anelloviridae family vector (e.g., anellovector) from the host cell, e.g., into the surrounding supernatant. In some embodiments, the host cell is lysed for harvest of Anelloviridae family vector (e.g., anellovector) from the cell lysate. In some embodiments, an Anelloviridae family vector (e.g., anellovector) may be introduced to a host cell line grown to a high cell density. In some embodiments, a host cell is a 293 cell, eg. an Expi-293 cell or a 293T cell. In some embodiments, a host cell is a MOLT-4 cell.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an Anelloviridae family vector (e.g., anellovector) comprising the genetic element. In some embodiments, cells are suspended in 2S Chica buffers.

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the Anelloviridae family vector (e.g., anellovector). To this end, cell lines that express an Anelloviridae family vector (e.g., anellovector) polymerase protein may be utilized as appropriate host cells. Host cells may be similarly engineered to provide other viral functions or additional functions.

To prepare the Anelloviridae family vector (e.g., anellovector) disclosed herein, a genetic element construct may be used to transfect cells that provide Anelloviridae family vector (e.g., anellovector) proteins and functions required for replication and production. Alternatively, cells may be transfected with a second construct (e.g., a virus) providing Anelloviridae family vector (e.g., anellovector) proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the Anelloviridae family vector (e.g., anellovector). In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into Anelloviridae family vectors (e.g., anellovectors) using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

### Methods for Providing Protein(s) in Cis or Trans

In some embodiments (e.g., cis embodiments described herein), the genetic element construct further comprises one or more expression cassettes comprising a coding sequence for an Anellovirus ORF (e.g., an Anellovirus ORF1, ORF2, ORF2/2, ORF2/3, ORF1/1, or ORF1/2, or a functional fragment thereof). In embodiments, the genetic element construct comprises an expression cassette comprising a coding sequence for an Anellovirus ORF1, or a splice variant or functional fragment thereof. Such genetic element constructs, which comprise expression cassettes for the effector as well as the one or more Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more Anellovirus ORFs (e.g., an Anellovirus ORF1, ORF2, ORF2/2, ORF2/3, ORF1/1, or ORF1/2, or a functional fragment thereof). In embodiments, the genetic element construct does not comprise an expression cassette comprising a coding sequence for an Anellovirus ORF1, or a splice variant or functional fragment thereof. Such genetic element constructs, which comprise expression cassettes for the effector but lack expression cassettes for one or more Anellovirus ORFs (e.g., Anellovirus ORF1 or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells comprising such genetic element constructs are incapable of enclosure of the genetic elements within proteinaceous exteriors in the absence of an additional nucleic construct encoding an Anellovirus ORF1 molecule. In other words, such genetic element constructs may be used for trans anellovector production methods in host cells, e.g., as described herein.

In some embodiments (e.g., cis embodiments described herein), the genetic element construct further comprises one or more expression cassettes comprising a coding sequence for one or more non-Anellovirus ORF (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector as well as the one or more non-Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more non-Anellovirus ORFs (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector but lack expression cassettes for one or more non-Anellovirus ORFs (e.g., a non-Anellovirus Rep molecule, e.g., an AAV Rep molecule, e.g., an AAV Rep protein, e.g., an AAV Rep2 protein), may be introduced into host cells. Host cells comprising such genetic element constructs may, in some instances, require additional nucleic acid constructs or integration of expression cassettes into the host cell genome for production of one or more components of the anellovector (e.g., for replication of the genetic element). In some embodiments, host cells comprising such genetic element constructs are incapable of replicating the genetic elements in the absence of an additional nucleic construct, e.g., encoding a non-Anellovirus Rep molecule, e.g., an AAV Rep molecule, e.g., an AAV Rep protein, e.g., an AAV Rep2 protein. In other words, such genetic element constructs may be used for trans anellovector production methods in host cells, e.g., as described herein.

### Exemplary Cell Types

Exemplary host cells suitable for production of Anelloviridae family vector (e.g., anellovector) include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, an eye cell (e.g., a photoreceptor cell, a retinal cell, a cell of the posterior eye cup (PEC), retinal ganglion cell, a cell of the optic nerve, a cell of the optic nerve head, or a retinal pigmented epithelium (RPE) cell), a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a mammalian cell (e.g., a human cell). In certain embodiments, a vector plasmid comprising a genetic element sequence flanked by recombinase recognition sites (e.g., lox sites or attB and attP sites, e.g., as described herein) is introduced into a mammalian host cell. In certain embodiments, the host cell comprises a site-specific recombinase (e.g., Cre or Bxb1, e.g., as described herein) capable of recombining the vector plasmid at the recombinase recognition sites, e.g., to produce a minicircle comprising the the genetic element sequence. In certain embodiments, the host cell further comprises a plasmid encoding the site-specific recombinase (e.g., the same plasmid as the vector plasmid or a different plasmid from the vector plasmid, e.g., as described herein). In certain embodiments, the host cell further comprises a rescue construct encoding one or more Anelloviridae family viral proteins (e.g., Anellovirus ORF1, ORF2, and/or ORF3; or CAV VP1, VP2, and/or VP3), e.g., an SRR plasmid as described herein. In certain embodiments, the minicircle is converted to a circular single-stranded DNA (cssDNA) comprising the sequence of the genetic element, e.g., in the host cell. In certain embodiments, the host cell comprises ORF1 molecules or VP1 molecules that can form a proteinaceous exterior encapsulating the cssDNA, thereby forming an Anelloviridae family vector particle.

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell. In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In some embodiments, the host cell is a 293 cell (e.g., a HEK293 cell, a HEK293T cell, or an Expi-293 cell). In some embodiments, the host cell is an Expi-293F cell.

In an aspect, the present disclosure provides a method of manufacturing an Anelloviridae family vector (e.g., anellovector) comprising a genetic element enclosed in a proteinaceous exterior, the method comprising providing a Expi-293 cell comprising an Anelloviridae family vector (e.g., anellovector) genetic element, and incubating the Expi-293 cell under conditions that allow the Anelloviridae family vector (e.g., anellovector) genetic element to become enclosed in a proteinaceous exterior in the Expi-293 cell. In some embodiments, the Expi-293 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the Expi-293 cell, e.g., from a genetic element construct (e.g., as described herein). In some embodiments, the method further comprises introducing the Anelloviridae family vector (e.g., anellovector) genetic element construct into the Expi-293 cell.

In an aspect, the present disclosure provides a method of manufacturing an Anelloviridae family vector (e.g., anellovector) comprising a genetic element enclosed in a proteinaceous exterior, the method comprising providing a MOLT-4 cell comprising an Anelloviridae family vector (e.g., anellovector) genetic element, and incubating the MOLT-4 cell under conditions that allow the Anelloviridae family vector (e.g., anellovector) genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-4 cell, e.g., from a genetic element construct (e.g., as described herein). In some embodiments, the method further comprises introducing the Anelloviridae family vector (e.g., anellovector) genetic element construct into the MOLT-4 cell.

In an aspect, the present disclosure provides a method of manufacturing an Anelloviridae family vector (e.g., anellovector) comprising a genetic element enclosed in a proteinaceous exterior, the method comprising providing a MOLT-3 cell comprising an Anelloviridae family vector (e.g., anellovector) genetic element, and incubating the MOLT-3 cell under conditions that allow the Anelloviridae family vector (e.g., anellovector) genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the Anelloviridae family vector (e.g., anellovector) genetic element is produced in the MOLT-3 cell, e.g., from a genetic element construct (e.g., as described herein). In some embodiments, the method further comprises introducing the Anelloviridae family vector (e.g., anellovector) genetic element construct into the MOLT-3 cell.

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the Anelloviridae family vector (e.g., anellovector) is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

In some embodiments, the host cell is a bacterial cell (e.g., an E. coli cell). In certain embodiments, a vector plasmid comprising a genetic element sequence flanked by recombinase recognition sites (e.g., attB and attP sites as described herein) is introduced into a bacterial host cell. In certain embodiments, the bacterial host cell comprises a site-specific recombinase (e.g., PhiC31 as described herein) capable of recombining the vector plasmid at the recombinase recognition sites, e.g., to produce a minicircle comprising the the genetic element sequence. In certain embodiments, the site-specific recombinase is encoded in the bacterial genome. In other embodiments, the site-specific recombinase is encoded in a second plasmid in the host cell. In certain embodiments, the minicircle is isolated from the bacterial cell (e.g., using miniprep, midipre, or maxiprep methods known in the art).

### Culture Conditions

Host cells comprising a genetic element and components of a proteinaceous exterior can be incubated under conditions suitable for enclosure of the genetic element within the proteinaceous exterior, thereby producing an anellovector. Suitable culture conditions include those described, e.g., in any of Examples 14-16. In some embodiments, the host cells are incubated in liquid media (e.g., Grace's Supplemented (TNM-FH), IPL-41, TC-100, Schneider's Drosophila, SF-900 II SFM, or and EXPRESS-FIVET^{M} SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release Anelloviridae family vectors (e.g., anellovectors) produced therein into the surrounding supernatant.

The production of Anelloviridae family vector (e.g., anellovector)-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of Anelloviridae family vectors (e.g., anellovectors).

### Harvest

Anelloviridae family vectors (e.g., anellovectors) produced by host cells can be harvested, e.g., according to methods known in the art. For example, Anelloviridae family vectors (e.g., anellovectors) released into the surrounding supernatant by host cells in culture can be harvested from the supernatant (e.g., as described in Example 14). In some embodiments, the supernatant is separated from the host cells to obtain the Anelloviridae family vectors (e.g., anellovectors). In some embodiments, the host cells are lysed before or during harvest. In some embodiments, the host cells are lysed in a detergent (e.g., Triton, e.g., 0.01%-0.1% Triton). In some embodiments, the Anelloviridae family vectors (e.g., anellovectors) are harvested from the host cell lysates (e.g., as described in Example 10 of PCT Publication No. WO 2020/123816, incorporated by reference herein in its entirety). In some embodiments, the Anelloviridae family vectors (e.g., anellovectors) are harvested from both the host cell lysates and the supernatant. In some embodiments, the purification and isolation of Anelloviridae family vectors (e.g., anellovectors) is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the Anelloviridae family vector (e.g., anellovector) may be harvested and/or purified by separation of solutes based on biophysical properties, e.g., ion exchange chromatography or tangential flow filtration, prior to formulation with a pharmaceutical excipient.

### In Vitro Assembly Methods for Anellovectors

An anellovector may be produced, e.g., by in vitro assembly, e.g., in the absence of a host cell, in a cell-free suspension, or in a supernatant. In some embodiments, the genetic element is contacted to an ORF1 molecule in vitro, e.g., under conditions that allow for assembly. In some embodiments, the genetic element is produced using a recombinase-based system as described herein. For example, circular double-stranded DNA minicircles comprising the sequence of a genetic element can be produced in bacterial cells by site-specific recombination of a vector plasmid (e.g., as described herein) comprising a genetic element sequence flanked by recombination recognition sites. Such minicircles can then be isolated from the bacterial cells (e.g., as described herein), and then converted to circular single stranded DNA (cssDNA) as described herein (e.g., by nicking one strand of the minicircle and selectively digesting the nicked strand, e.g., as described herein). This yields a cssDNA comprising the sequence of the genetic element, which can itself serve as a genetic element suitable for encapsulation within a proteinaceous exterior comprising Anelloviridae family viral capsid proteins (e.g., ORF1 molecules or VP1 molecules) to form an Anelloviridae family vector particle.

In an aspect, the present disclosure provides a particle (e.g., an anellovector as described herein) produced via in vitro assembly (e.g., as described herein). The particle may, in some instances, comprise a proteinaceous exterior comprising an ORF1 molecule and a genetic element encoding an exogenous effector, which is enclosed within the proteinaceous exterior. In some embodiments, a particle produced by in vitro assembly does not include a substantial (e.g., detectable) amount of one or more constituents (e.g., small molecules, peptides, polypeptides, nucleic acids, polynucleotides, lipids, sugars, and/or organelles) from a host cell (e.g., a host cell used to produce the ORF1 molecules and/or the genetic element). In some embodiments, the particle may have one or more (e.g., 1, 2, 3, 4, 5, 6, 7, 8, or all 9) of the following characteristics:
(i) the genetic element (e.g., a DNA genetic element) does not comprise an Anellovirus 5′ UTR and/or an Anellovirus origin of replication;
(ii) the sequence encoding the exogenous effector takes up at least 90%, 95%, 96%, 97%, 98%, 99% or 100% of the genetic element (e.g., a DNA genetic element);
(iii) the heterologous nucleic acid sequence takes up at least 90%, 95%, 96%, 97%, 98%, 99% or 100% of the genetic element (e.g., a DNA genetic element);
(iv) the particle does not comprise a detectable amount of (e.g., any) polypeptides from a host cell, or comprises less than 5, 10, 15, 20, 25, 30, 40, or 50 copies of a polypeptide from a host cell;
(v) the particle does not comprise a detectable amount of (e.g., any) nucleic acid molecules from a host cell other than the genetic element (or copies thereof), or comprises less than 2, 3, 4, or 5 copies of a nucleic acid molecule from a host cell;
(vi) the particle comprises a denaturant in a concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M;
(vii) does not substantially replicate when introduced into a cell (e.g., a human cell); and/or
(viii) has a symmetrical morphology and/or a diameter of at least 30, 31, 32, 33, 34, or 35 nm.

In another aspect, the disclosure provides a population of the particles (e.g., the anellovectors).

In some embodiments, Anellovirus proteins to be used for in vitro assembly of a particle (e.g., an anellovector) as described herein are produced in a cell. In some embodiments, baculovirus constructs are used to produce Anellovirus proteins (e.g., one or more of an Anellovirus ORF1, ORF2, and/or ORF3 molecule, e.g., as described herein), for example, in insect cells (e.g., Sf9 cells). Anelloviridae family virus (e.g., Anellovirus) proteins. These proteins may then be used, e.g., for in vitro assembly to encapsidate a genetic element, e.g., a genetic element comprising RNA. In some embodiments, a polynucleotide encoding one or more Anelloviridae family virus (e.g., Anellovirus) protein is fused to a promoter for expression in a host cell, e.g., an insect cell or an animal cell. In some embodiments, the polynucleotide is cloned into a baculovirus expression system. In some embodiments, a host cell, e.g., an insect cell, is infected with the baculovirus expression system and incubated for a period of time under conditions suitable for expression of the one or more Anellovirus proteins. In some embodiments, an infected cell is incubated for about 1, 2, 3, 4, 5, 10, 15, or 20 days. In some embodiments, an infected cell is lysed to recover the one or more Anellovirus proteins.

In some embodiments, an Anellovirus protein (e.g., an Anellovirus ORF1 molecule) is produced as described in Example 7. In some embodiments, an Anellovirus protein (e.g., an Anellovirus ORF1 molecule) is produced in insect cells as described in Example 8 or 10. In some embodiments, a plurality of Anellovirus ORF1 molecules has a propensity to self-assemble into a proteinaceous exterior, for example, to form a virus-like particle (VLP). In certain embodiments, the VLPs do not encapsulate a genetic element as described herein. In certain embodiments, the VLP comprises at least 40, 45, 50, 55, 60, 65, or 70 ORF1 molecules in its proteinaceous exterior.

In certain embodiments, a VLP comprising an Anellovirus ORF1 molecule can be denatured as described herein (e.g., using a chaotropic agent, such as urea). In embodiments, a VLP is denatured using one or more of: buffers of different pH, conditions of defined conductivity (salt content), a detergent (such as SDS (e.g., 0.1% SDS), Tween, Triton), a chaotropic agent (such as urea, e.g., as described herein), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving defined temperature and time (reannealing temperatures), for example, as described in Example 12. In embodiments, a VLP is denatured using urea as described in Example 11. In embodiments, a VLP is denatured in urea at a concentration of about 1-10 M (e.g., about 1-2 M, 2-3 M, 3-4 M, 4-5 M, 5-6 M, 6-7 M, 7-8 M, 8-9 M, 9-10M, or 1-6 M). In embodiments, a VLP is denatured in urea at a concentration of about 1-10 M (e.g., about 1, 2, 3, 4, 5, 6, 7, 8, 9, or 10M). In an embodiment, a VLP is denatured in urea at a concentration of 2 M. In embodiments, a VLP is denatured under high salt conditions. In embodiments, denaturation of the VLP results in the ORF1 molecules forming capsomers (e.g., capsomeric decamers comprising about 10 copies of the ORF1 molecule).

In some embodiments, removing the capsomers from the presence of the chaotropic agent (e.g., by purifying the capsomers or removing the chaotropic agent, e.g., by dilution or dialysis) allows the ORF1 molecules to reform VLPs (e.g., VLPs comprising at least 40, 45, 50, 55, 60, 65, or 70 ORF1 molecules). In embodiments, VLPs are reformed by dialyzing out a chaotropic agent (e.g., urea) as described in Example 11. In some embodiments, VLPs are reformed in the presence of a cargo of interest, such as a genetic element (e.g., as described herein), under conditions suitable for enclosure of the cargo in the proteinaceous exterior of the VLP (e.g., as described in Example 12). In some embodiments, an isolated Anellovirus protein (e.g., an ORF1 molecule) is purified (e.g., from a cell). In some embodiments, an Anellovirus protein is purified using purification techniques including but not limited to chelating purification, heparin purification, gradient sedimentation purification, and/or SEC purification. In embodiments, an Anellovirus protein is purified as described in Example 7. In some embodiments, an Anellovirus protein (e.g., an Anellovirus ORF1 molecule) is purified from insect cells as described in Example 8 or 10. In some embodiments, a purified Anellovirus protein is mixed with a genetic element to encapsidate the genetic element, e.g., a genetic element comprising RNA. In some embodiments, a genetic element is encapisdated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding Anellovirus ORF1 may be untagged. In some embodiments, DNA encoding Anellovirus ORF1 may contain tags fused N-terminally and/or C-terminally. In some embodiments, DNA encoding Anellovirus ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding Anellovirus ORF1 may be expressed alone or in combination with any number of helper proteins. In some embodiments, DNA encoding Anellovirus ORF1 is expressed in combination with Anellovirus ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical structural jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another Anellovirus ORF1 protein (e.g., structural jellyroll fragments or the C-terminal portion of Ring 2 ORF1 replaced with comparable portions of Ring 9 ORF1.

In some embodiments, a genetic element to be used for in vitro assembly of a particle (e.g., an anellovector) as described herein are produced in a cell. In some embodiments, a cell is transfected with a construct (e.g., a plasmid, tandem construct, and/or an in vitro circularized nucleic acid molecule, e.g., as described herein) comprising the sequence of a genetic element. In embodiments, the cell is incubated under conditions suitable for replication of the construct. In embodiments, the cell is incubated under conditions suitable for production and/or replication of the genetic element (e.g., from the construct). In embodiments, the cell is lysed to recover the genetic element. In some embodiments, a genetic element to be used for in vitro assembly is a DNA, e.g., a single-stranded DNA (ssDNA). In embodiments, the genetic element is a negative sense ssDNA (e.g., generated as described in Example 6). In some embodiments, a genetic element to be used for in vitro assembly comprises RNA (e.g., an mRNA), for example, as described in Example 12. In some embodiments, a genetic element to be used for in vitro assembly comprises RNA (e.g., an mRNA) and the ORF1 molecule of the proteinaceous exterior comprises one or more contact residues that binds RNA (e.g., a domain from an RNA-binding protein, e.g., an mRNA-binding protein, e.g., MS2 coat protein), for example, as described in Example 12. In some embodiments, a genetic element to be used for in vitro assembly comprises RNA (e.g., an mRNA) and DNA (e.g., a DNA portion comprising a sequence that binds to an Anellovirus ORF1 molecule), for example, as described in Example 12. In certain embodiments, the RNA portion of the genetic element is covalently bound to the DNA portion of the genetic element. In certain embodiments, the RNA portion of the genetic element is hybridized to the DNA portion of the genetic element. In embodiments, the RNA portion of the genetic element comprises a region capable of hybridizing to (e.g., complementary to) at least a subsequence of the DNA portion of the genetic element.

In some embodiments, the present disclosure describes a method of making an anellovector, the method comprising: (a) providing a mixture comprising: (i) a genetic element (e.g., a genetic element comprising DNA and/or RNA), and (ii) an ORF1 molecule; and (b) incubating the mixture under conditions suitable for enclosing the genetic element within a proteinaceous exterior comprising the ORF1 molecule, thereby making an anellovector; wherein the mixture is not comprised in a cell. In some embodiments, the anellovector is assembled in vitro as described in Example 7. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

In Vitro Assembly Methods for anelloVLPs

An anelloVLP as described herein may be produced, e.g., by in vitro assembly, e.g., in a cell-free suspension or in a supernatant. In some embodiments, the anelloVLP is produced by contacting a plurality of ORF1 molecules (e.g., in capsomers) to an effector (e.g., an exogenous effector) in vitro, e.g., under conditions that allow for assembly. In some embodiments, the plurality of ORF1 molecules enclose the effector in a proteinaceous exterior. In some embodiments, the effector is attached to the exterior surface of a proteinaceous exterior formed from the plurality of ORF1 molecules (e.g., as a surface moiety as described herein). In some embodiments, production of an anelloVLP comprises expressing a plurality of ORF1 molecules in a cell, purifying the plurality of ORF1 molecules, denaturing virus-like particles formed by the ORF1 molecules (e.g., as described herein), and then allowing the ORF1 molecules to reform virus-like particles in the presence of an effector (e.g., an exogenous effector), thereby forming anelloVLPs comprising the ORF1 molecules enclosing the effector.

In an aspect, the present disclosure provide a particle (e.g., an anelloVLP as described herein) produced via in vitro assembly (e.g., as described herein). The particle may, in some instances, comprise a proteinaceous exterior comprising an ORF1 molecule and an effector (e.g., an exogenous effector), e.g., as described herein. In some embodiments, the effector is enclosed within the proteinaceous exterior. In some embodiments, the effector is comprised in a surface moiety (e.g., as described herein) attached to the exterior surface of the proteinaceous exterior. In some embodiments, a particle produced by in vitro assembly does not include a substantial (e.g., detectable) amount of one or more constituents (e.g., small molecules, peptides, polypeptides, nucleic acids, polynucleotides, lipids, sugars, and/or organelles) from a host cell (e.g., a host cell used to produce the ORF1 molecules and/or the genetic element). In some embodiments, the particle may have one or more (e.g., 1, 2, 3, 4, or all 5) of the following characteristics:
(i) does not comprise (e.g., does not enclose) a polynucleotide,
(ii) does not comprise (e.g., does not enclose) detectable levels of polynucleotides,
(iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.

In another aspect, the disclosure provides a population of the particles (e.g., the anelloVLPs). In some embodiments, the population further comprises one or more anellovectors. In some embodiments, the population does not comprise anellovectors.

In some embodiments, Anellovirus proteins to be used for in vitro assembly of a particle (e.g., an anelloVLP) as described herein are produced in a cell. In some embodiments, baculovirus constructs are used to produce Anellovirus proteins (e.g., one or more of an Anellovirus ORF1, ORF2, and/or ORF3 molecule, e.g., as described herein), for example, in insect cells (e.g., Sf9 cells). These proteins may then be used, e.g., for in vitro assembly to form an anelloVLP, e.g., as described herein. In some embodiments, a polynucleotide encoding one or more Anellovirus proteins is fused to a promoter for expression in a host cell, e.g., an insect cell or an animal cell. In some embodiments, the polynucleotide is cloned into a baculovirus expression system. In some embodiments, a host cell, e.g., an insect cell, is infected with the baculovirus expression system and incubated for a period of time under conditions suitable for expression of the one or more Anellovirus proteins. In some embodiments, an infected cell is incubated for about 1, 2, 3, 4, 5, 10, 15, or 20 days. In some embodiments, an infected cell is lysed to recover the one or more Anellovirus proteins.

In some embodiments, an Anellovirus protein (e.g., an Anellovirus ORF1 molecule) is produced as described in Example 7. In some embodiments, an Anellovirus protein (e.g., an Anellovirus ORF1 molecule) is produced in insect cells as described in Example 8 or 10. In some embodiments, a plurality of Anellovirus ORF1 molecules has a propensity to self-assemble into a proteinaceous exterior, for example, to form a virus-like particle (VLP). In certain embodiments, the VLPs do not encapsulate a genetic element as described herein. In certain embodiments, the VLPs do not encapsulate an effector to be delivered to a target cell, e.g., as described herein. In certain embodiments, the VLP comprises at least 40, 45, 50, 55, 60, 65, or 70 ORF1 molecules in its proteinaceous exterior.

In certain embodiments, a VLP comprising an Anellovirus ORF1 molecule can be denatured as described herein (e.g., using a chaotropic agent, such as urea). In embodiments, a VLP is denatured using one or more of: buffers of different pH, conditions of defined conductivity (salt content), a detergent (such as SDS (e.g., 0.1% SDS), Tween, Triton), a chaotropic agent (such as urea, e.g., as described herein), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving defined temperature and time (reannealing temperatures), for example, as described in Example 12. In embodiments, a VLP is denatured using urea as described in Example 11. In embodiments, a VLP is denatured in urea at a concentration of about 1-10 M (e.g., about 1-2 M, 2-3 M, 3-4 M, 4-5 M, 5-6 M, 6-7 M, 7-8 M, 8-9 M, 9-10M, or 1-6 M). In embodiments, a VLP is denatured in urea at a concentration of about 1-10 M (e.g., about 1, 2, 3, 4, 5, 6, 7, 8, 9, or 10M). In an embodiment, a VLP is denatured in urea at a concentration of 2 M. In embodiments, a VLP is denatured under high salt conditions. In embodiments, denaturation of the VLP results in the ORF1 molecules forming capsomers (e.g., capsomeric decamers comprising about 10 copies of the ORF1 molecule).

In some embodiments, removing the capsomers from the presence of the chaotropic agent (e.g., by purifying the capsomers or removing the chaotropic agent, e.g., by dilution or dialysis) allows the ORF1 molecules to reform VLPs (e.g., VLPs comprising at least 40, 45, 50, 55, 60, 65, or 70 ORF1 molecules). In embodiments, VLPs are reformed by dialyzing out a chaotropic agent (e.g., urea) as described in Example 11. In some embodiments, VLPs are reformed in the presence of a cargo of interest (e.g., an effector as described herein), for example, under conditions suitable for enclosure of the cargo in the proteinaceous exterior of the VLP (e.g., as described in Example 12). In some embodiments, an isolated Anellovirus protein (e.g., an ORF1 molecule) is purified (e.g., from a cell). In some embodiments, an Anellovirus protein is purified using purification techniques including but not limited to chelating purification, heparin purification, gradient sedimentation purification, and/or SEC purification. In embodiments, an Anellovirus protein is purified as described in Example 7. In some embodiments, an Anellovirus protein (e.g., an Anellovirus ORF1 molecule) is purified from insect cells as described in Example 8 or 10. In some embodiments, a plurality of purified Anellovirus proteins are mixed with an effector. In embodiments, the purified Anellovirus proteins encapsulated the effector. In some embodiments, the effector is encapsulated using an ORF1 protein, ORF2 protein, or modified version thereof.

In some embodiments, DNA encoding Anellovirus ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding Anellovirus ORF1 may be untagged. In some embodiments, DNA encoding Anellovirus ORF1 may contain tags fused N-terminally and/or C-terminally. In some embodiments, DNA encoding Anellovirus ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding Anellovirus ORF1 may be expressed alone or in combination with any number of helper proteins. In some embodiments, DNA encoding Anellovirus ORF1 is expressed in combination with Anellovirus ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical structural jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another Anellovirus ORF1 protein (e.g., structural jellyroll fragments or the C-terminal portion of Ring 2 ORF1 replaced with comparable portions of Ring 9 ORF1.

In some embodiments, the present disclosure describes a method of making an anelloVLP, the method comprising: (a) providing a mixture comprising: (i) an effector (e.g., an exogenous effector), and (ii) an ORF1 molecule; and (b) incubating the mixture under conditions suitable for enclosing the effector within a proteinaceous exterior comprising the ORF1 molecule, thereby making an anelloVLP; wherein the mixture is not comprised in a cell. In some embodiments, the anelloVLP is assembled in vitro as described in Example 7. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anelloVLP composition, comprising: (a) providing a plurality of anelloVLPs or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anelloVLPs, e.g., as a pharmaceutical composition suitable for administration to a subject, e.g., if one or more of the parameters of (b) meet a specified threshold.

### Enrichment and Purification

Anellovectors or anelloVLPs produced as described herein can be further purified and/or enriched, e.g., to produce an anellovector preparation or an anelloVLP preparation, respectively. In some embodiments, the harvested anellovectors or anelloVLPs are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested anellovectors or anelloVLPs are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### III. Vectors

The genetic element described herein may be included in a vector. Suitable vectors as well as methods for their manufacture and their use are well known in the prior art.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid.

The genetic element or any of the sequences within the genetic element can be obtained using any suitable method. Various recombinant methods are known in the art, such as, for example screening libraries from cells harboring viral sequences, deriving the sequences from a vector known to include the same, or isolating directly from cells and tissues containing the same, using standard techniques. Alternatively or in combination, part or all of the genetic element can be produced synthetically, rather than cloned.

In some embodiments, the vector includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is substantially non-pathogenic and/or substantially non-integrating in a host cell or is substantially non-immunogenic in a host.

In some embodiments, the vector is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

An anellovector or or anelloVLP described herein may also be included in pharmaceutical compositions with a pharmaceutical excipient, e.g., as described herein. In some embodiments, the pharmaceutical composition comprises at least 10⁵, 10⁶, 10⁷, 10⁸, 10⁹, 10¹⁰, 10¹¹, 10¹², 10¹³, 10¹⁴, or 10¹⁵ anellovectors or anelloVLPs. In some embodiments, the pharmaceutical composition comprises about 10⁵-10¹⁵, 10⁵-10¹⁰, or 10¹⁰-10¹⁵ anellovectors or anelloVLPs. In some embodiments, the pharmaceutical composition comprises about 10⁸ (e.g., about 10⁵, 10⁶, 10⁷, 10⁸, 10⁹, or 10¹⁰) genomic equivalents/mL of the anellovector or anelloVLP. In some embodiments, the pharmaceutical composition comprises 10⁵-10¹⁰, 10⁶-10¹⁰, 10⁷-10¹⁰, 10⁸-10¹⁰, 10⁹-10¹⁰, 10⁵-10⁶, 10⁵-10⁷, 10⁵-10⁸, 10⁵-10⁹, 10⁵-10¹¹, 10⁵-10¹², 10⁵-10¹³, 10⁵-10¹⁴, 10⁵-10¹⁵, or 10¹⁰-10¹⁵ genomic equivalents/mL of the anellovector or anelloVLP. In some embodiments, the pharmaceutical composition comprises sufficient anellovectors or anelloVLPs to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the anellovectors or anelloVLPs per cell to a population of the eukaryotic cells. In some embodiments, the pharmaceutical composition comprises sufficient anellovectors or anelloVLPs to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the anellovectors or anelloVLPs per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates. In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an Anelloviridae family vector (e.g., anellovector) comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

In one aspect, the invention described herein includes a pharmaceutical composition comprising:
a) an anelloVLP as described herein; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an anellovector, anelloVLP, Anellovirus, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueeous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et a1. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Membrane Penetrating Polypeptides

In some embodiments, the composition further comprises a membrane penetrating polypeptide (MPP) to carry the components into cells or across a membrane, e.g., cell or nuclear membrane. Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the anellovector, anelloVLP, or composition described herein may also include one or more heterologous moieties. In one aspect, the anellovector or composition comprising an anellovector or anelloVLP described herein may also include one or more heterologous moiety in a fusion. In some embodiments, a heterologous moiety may be linked with the genetic element. In some embodiments, a heterologous moiety may be enclosed in the proteinaceous exterior as part of the anellovector or anelloVLP. In some embodiments, a heterologous moiety may be administered with the anellovector or anelloVLP.

In one aspect, the invention includes a cell or tissue comprising any one of the anellovectors or anelloVLP and heterologous moieties described herein.

In another aspect, the invention includes a pharmaceutical composition comprising an anellovector or anelloVLP and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein. In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

### Viruses

In some embodiments, the composition may further comprise a virus as a heterologous moiety, e.g., a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the anellovector or anelloVLP is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein, e.g., as listed in any one of Tables N1-N32.

In some embodiments, the heterologous moiety may comprise one or more viruses that are identified as lacking in the subject. For example, a subject identified as having dyvirosis may be administered a composition comprising an anellovector and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the anellovector or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective anellovector or virus under the control of regulatory sequences within the LTR. Suitable cell lines for replicating the anellovectors described herein include cell lines known in the art, e.g., A549 cells, which can be modified as described herein.

### Targeting Moiety

In some embodiments, the composition, anellovector, or anelloVLP described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the anellovector, or anelloVLP, or genetic element. For example, a targeting moiety may include a therapeutic that interacts with a specific molecule of interest to increase, decrease or otherwise modulate its function.

### Tagging or Monitoring Moiety

In some embodiments, the composition, anellovector, or anelloVLP described herein may further comprise a tag to label or monitor the anellovector, anelloVLP, or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

### Nanoparticles

In some embodiments, the composition, anellovector, or anelloVLP described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

### Small Molecules

In some embodiments, the composition, anellovector, or anelloVLP described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organomettallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5 (2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.g., PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

### Peptides or Proteins

In some embodiments, the composition, anellovector, or anelloVLP described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition, anellovector, or anelloVLP described herein includes a polypeptide linked to a ligand that is capable of targeting a specific location, tissue, or cell.

### Oligonucleotide Aptamers

In some embodiments, the composition, anellovector, or anelloVLP described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamer-cite_note-10), hemin, interferon γ, prostate specific antigen (PSA), dopamine, and the non-classical oncogene, heat shock factor 1 (HSF1).

### Peptide Aptamers

In some embodiments, the composition, anellovector, or anelloVLP described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### V. Host Cells

The invention is further directed to a host or host cell comprising an anellovector or anelloVLP as described herein. In some embodiments, the host or host cell is a plant, insect, bacteria, fungus, vertebrate, mammal (e.g., human), or other organism or cell. In certain embodiments, as confirmed herein, provided anellovectors infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the anellovector or anelloVLP is substantially non-immunogenic in the host. In certain embodiments, the anellovector or genetic element thereof, or the anelloVLP, fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an anellovector or anelloVLP. In some embodiments, the host is a mammal, such as a human. The amount of the anellovector or anelloVLP in the host can be measured at any time after administration. In certain embodiments, a time course of anellovector growth in a culture is determined.

In some embodiments, the anellovector, e.g., an anellovector as described herein, is heritable. In some embodiments, the anellovector is transmitted linearly in fluids and/or cells from mother to child. In some embodiments, daughter cells from an original host cell comprise the anellovector. In some embodiments, a mother transmits the anellovector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the anellovector in a host cell has a transmission efficiency during meiosis of at 25%, 50%, 60%, 70%, 80%, 85%, 90%, 95%, or 99%. In some embodiments, the anellovector in a host cell has a transmission efficiency during mitosis of at least 25%, 50%, 60%, 70%, 80%, 85%, 90%, 95%, or 99%. In some embodiments, the anellovector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the anellovector, e.g., anellovector replicates within the host cell. In one embodiment, the anellovector is capable of replicating in a mammalian cell, e.g., human cell. In other embodiments, the anellovector is replication deficient or replication incompetent.

While in some embodiments the anellovector replicates in the host cell, the anellovector does not integrate into the genome of the host, e.g., with the host's chromosomes. In some embodiments, the anellovector has a negligible recombination frequency, e.g., with the host's chromosomes. In some embodiments, the anellovector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The anellovectors and compositions comprising anellovectors or anelloVLPs described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. In some embodiments, an Anelloviridae family vector, e.g., anellovector, or pharmaceutical composition as described herein is administered subretinally. In some embodiments, an Anelloviridae family vector, e.g., anellovector, or pharmaceutical composition as described herein is administered intravitreally. In some embodiments, an Anelloviridae family vector, e.g., anellovector, or pharmaceutical composition as described herein is administered suprachoroidally. The anellovectors may be administered alone or formulated as a pharmaceutical composition.

The anellovectors or anelloVLP may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of an Anelloviridae family vector (e.g., anellovector) or composition comprising same, e.g., as described herein, may result in delivery of a genetic element comprised by the Anelloviridae family vector (e.g., anellovector) to a target cell, e.g., in a subject.

An anellovector or anelloVLP, or composition thereof, described herein, e.g., comprising an effector (e.g., an endogenous or exogenous effector), may be used to deliver the effector to a cell, tissue, or subject. In some embodiments, the effector is a therapeutic effector. In some embodiments, the Anelloviridae family vector (e.g., anellovector) or composition thereof is used to deliver the effector to the eye of a subject, e.g., a mammalian subject, e.g., a human subject. In some embodiments, the Anelloviridae family vector (e.g., anellovector) or composition thereof is used to deliver the effector to a cell of the eye of a subject, e.g., a mammalian subject, e.g., a human subject. In certain embodiments, the cell of the eye is a photoreceptor cell, a retinal cell, a cell of the posterior eye cup (PEC), retinal ganglion cell, a cell of the optic nerve, a cell of the optic nerve head, or a retinal pigmented epithelium (RPE) cell. In some embodiments, the Anelloviridae family vector (e.g., anellovector) or composition thereof is used to deliver the effector to bone marrow, blood, heart, GI or skin. Delivery of an effector by administration of an anellovector composition or anelloVLP composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector or anelloVLP, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, an anellovector, anelloVLP, or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the Anelloviridae family vector (e.g., anellovector) or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with an anellovector or anelloVLP described herein, or a composition comprising the anellovector or anelloVLP, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector or anelloVLP modulates (e.g., increases or decreases) an activity or function in a cell with which the anellovector is contacted. In some embodiments, the anellovector or anelloVLP modulates (e.g., increases or decreases) the level or activity of a molecule (e.g., a nucleic acid or a protein) in a cell with which the anellovector or anelloVLP is contacted. In some embodiments, the anellovector or anelloVLP decreases viability of a cell, e.g., a cancer cell, with which the anellovector or anelloVLP is contacted, e.g., by at least about 10%, 20%, 30%, 40%, 50%, 60%, 70%, 80%, 90%, 95%, 99%, or more. In some embodiments, the anellovector or anelloVLP comprises an effector, e.g., an miRNA, e.g., miR-625, that decreases viability of a cell, e.g., a cancer cell, with which the anellovector or anelloVLP is contacted, e.g., by at least about 10%, 20%, 30%, 40%, 50%, 60%, 70%, 80%, 90%, 95%, 99%, or more. In some embodiments, the anellovector or anelloVLP increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector or anelloVLP comprises an effector, e.g., an miRNA, e.g., miR-625, that increases apoptosis of a cell, e.g., a cancer cell, e.g., by increasing caspase-3 activity, with which the anellovector or anelloVLP is contacted, e.g., by at least about 10%, 20%, 30%, 40%, 50%, 60%, 70%, 80%, 90%, 95%, 99%, or more.

### Methods of Treatment (e.g., for Diseases or Disorders of the Eye)

In one aspect, the present disclosure provides a method for treating disease, disorder, or condition (e.g., a disease of the eye), the method comprising administering a pharmaceutically effective amount of an Anelloviridae family vector or a pharmaceutical composition comprising an Anelloviridae family vector provided herein to a subject (e.g., a human subject) in need of such treatment.

In some embodiments, a disease, disorder, or condition that can be treated with the Anelloviridae family vector (e.g., anellovector) described herein, or a composition comprising the Anelloviridae family vector (e.g., anellovector), is a disease of the eye.

In some embodiments, the disease is selected from the group of ocular neovascular diseases consisting of: age-related macular degeneration (AMD), wet-AMD, dry-AMD, retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy and diabetic retinal edema.

In some embodiments, a disease, disorder, or condition that can be treated with the Anelloviridae family vector (e.g., anellovector) described herein, or a composition comprising such an Anelloviridae family vector, is a monogenic disease.

In some embodiments, a disease, disorder, or condition that can be treated with the Anelloviridae family vector (e.g., anellovector) described herein, or a composition comprising such an Anelloviridae family vector, is a polygenic disease (e.g., glaucoma).

In some embodiments, a disease, disorder, or condition that can be treated with the Anelloviridae family vector (e.g., anellovector) described herein, or a composition comprising such an Anelloviridae family vector, is a macular degeneration (e.g., age-related macular degeneration (AMD), Stargardt disease, or myopic macular degeneration). In certain embodiments, the macular degeneration is wet AMD. In certain embodiments, the macular degeneration is dry AMD (e.g., AMD with geographic atrophy).

In some embodiments, a disease, disorder, or condition that can be treated with the Anelloviridae family vector (e.g., anellovector) described herein, or a composition comprising such an Anelloviridae family vector, is a retinal disease. In certain embodiments, the retinal disease is an inherited retinal disease (IRD), e.g., as described in Stone et al. (2017, Ophthalmology; incorporated herein by reference with respect to diseases and disorders described therein). In certain embodiments, the retinal disease is retinitis pigmentosa (e.g., X-linked retinitis pigmentosa (XLRP).

In some embodiments, a disease, disorder, or condition that can be treated with the Anelloviridae family vector (e.g., anellovector) described herein, or a composition comprising such an Anelloviridae family vector, is a VEGF-associated disorder (e.g., a cancer, e.g., as described herein; a macular edema; or a proliferative retinopathy).

In some embodiments, the disease, disorder, or conditionis selected from the group consisting of: retinal leakage, Leber congenital amaurosis (LCA) (e.g., wherein the genetic element comprises a human RPE65 sequence, e.g., a sequence encoding a human RPE65 protein, or an amino acid sequence having at least 75%, 80%, 85%, 90%, 95%, 96%, 97%, 98%, or 99% sequence identity thereto), amaurosis congenita, cone rod dystrophy, choroideremia, vitelliform macular dystrophy, hyperferritinemia-cataract syndrome, optic atrophy, XLR retinoschisis, cytomegalovirus retinitis, achromatopsia, Leber hereditary optical neuropathy, keratitis, uveitis, Grave's opthalmolopathy, diabetic retinopathy, or diabetic macular edema.

In some cases, dry AMD may be treated. In some cases, dry AMD may be referred to as central geographic atrophy, characterized by atrophy of the retinal pigment epithelial later below the retina and subsequent loss of photoreceptors in the central part of the eye. The composition and methods of this disclosure provide for the treatment of any and all forms of AMD.

In another aspect, the present disclosure provides a method for prophylactic treatment of AMD or ocular neovascular diseases as described herein, comprising administering a pharmaceutically effective amount of the pharmaceutical compositions provided herein to a human subject in need of such treatment. The present disclosure may be used to treat patients at risk of developing AMD, or presenting early symptoms of the disease. This may include treatment of eyes either simultaneously or sequentially. Simultaneous treatment may mean that the treatment is administered to each eye at the same time or that both eyes are treated during the same visit to a treating physician or other healthcare provider. It has been documented that patients have a higher risk of developing AMD in a healthy fellow eye of an eye that presents symptoms of AMD, or in patients who have a genetic predisposition toward developing AMD. The present disclosure can be used as a prophylactic treatment in prevention of AMD in the fellow eye. While the mechanism underlying the increased risk for the progression of ocular neovascular disease in a fellow eye is unknown, there are multiple studies in the art detailing this elevated risk. For example, in one such large scale study, of 110 fellow eyes observed that progressed to advanced AMD, choroidal neovascularization (CNV) developed in 98 eyes and foveal geographic atrophy (GA) in 15 eyes. Ophthalmologica 2011; 226(3):110-8. doi: 10.1159/000329473. Curr Opin Ophthalmol. 1998 June; 9(3):38-46. No non-ocular characteristic (age, gender, history of hypertension or smoking) or ocular feature of the study eye at baseline (lesion composition, lesion size, or visual acuity) was predictive of progression to advanced AMD in this cohort. However, statistical analysis indicates that AMD symptoms of the first eye, including drusen size, focal hyperpigmentation, and nonfoveal geographic atrophy had significant independent relationships in assessing risk of developing of AMD in the fellow eye. Recent studies have indicated that of ocular characteristics, genetic factors and certain environmental factors may play a role in the increased risk of developing AMD in the fellow eye. JAMA Ophthalmol. 2013 Apr. 1; 131(4):448-55. doi: 10.1001/jamaophthalmol.2013.2578. Given the well characterized elevated risk of AMD development in untreated fellow eyes, there is need in the art of methods for preventing onset and subsequent vision loss due to the disease.

In some aspects, no vector is detected in the human subject's tear, blood, saliva or urine samples 7, 14, 21 or 30 days after administering said pharmaceutical composition. In some aspects, the presence of the viral vector is detected by qPCR or ELISA as known in the art.

In some aspects, the human subject shows no clinically significant retinal toxicity as assessed by serial ophthalmic examinations over at least about a 2, 3, 4, 5, 6, 7, 8, 9, 10, 11 or 12 month months period. In some aspects, the human subject shows no clinically significant retinal toxicity as assessed by serial ophthalmic examinations over at most about a 2, 3, 4, 5, 6, 7, 8, 9, 10, 11 or 12 month months period.

In some aspects, no superficial, anterior segment or vitreous inflammatory signs are present in the human subject over at least a two months period. In some cases, no superficial, anterior segment or vitreous inflammatory signs are present in the human subject at 1 week or at 3, 6, 9 or 12 months after administration of the pharmaceutical composition.

In some aspects, there is no evidence of visual acuity loss, TOP elevation, retinal detachment, or any intraocular or systemic immune response in said human subject at least 120 days post administration.

In some embodiments, a diseases, disorder, or condition that can be treated with the Anelloviridae family vector (e.g., anellovector) described herein, or a composition comprising the Anelloviridae family vector (e.g., anellovector), is a disease of the eye.

In some embodiments, the disease of the eye is selected from the group consisting of: neovascular age-related macular degeneration (nAMD) (also known as wet AMD or WAMD), dry AMD, retinal vein occlusion (RVO), diabetic macular edema (DME), or diabetic retinopathy (DR) (in particular, wet AMD), comprising delivering to the retina or to the posterior eye cup (PEC) of said human subject a therapeutically effective amount of anti-hVEGF antigen-binding fragment. In some embodiments, a disease, disorder, or condition that can be treated with the Anelloviridae family vector (e.g., anellovector) described herein, or a composition comprising such an Anelloviridae family vector, is a monogenic disease.

In some embodiments, a disease, disorder, or condition that can be treated with the Anelloviridae family vector (e.g., anellovector) described herein, or a composition comprising such an Anelloviridae family vector, is a polygenic disease (e.g., glaucoma).

In some embodiments, a disease, disorder, or condition that can be treated with the Anelloviridae family vector (e.g., anellovector) described herein, or a composition comprising such an Anelloviridae family vector, is a macular degeneration (e.g., age-related macular degeneration (AMD), Stargardt disease, or myopic macular degeneration). In certain embodiments, the macular degeneration is wet AMD. In certain embodiments, the macular degeneration is dry AMD (e.g., AMD with geographic atrophy).

In some embodiments, a disease, disorder, or condition that can be treated with the Anelloviridae family vector (e.g., anellovector) described herein, or a composition comprising such an Anelloviridae family vector, is a retinal disease. In certain embodiments, the retinal disease is an inherited retinal disease (IRD), e.g., as described in Stone et al. (2017, Ophthalmology; incorporated herein by reference with respect to diseases and disorders described therein). In certain embodiments, the retinal disease is retinitis pigmentosa (e.g., X-linked retinitis pigmentosa (XLRP).

In some embodiments, a disease, disorder, or condition that can be treated with the Anelloviridae family vector (e.g., anellovector) described herein, or a composition comprising such an Anelloviridae family vector, is a VEGF-associated disorder (e.g., a cancer, e.g., as described herein; a macular edema; or a proliferative retinopathy).

In some embodiments, the disease, disorder, or condition is selected from the group consisting of: retinal leakage, Leber congenital amaurosis (LCA) (e.g., wherein the genetic element comprises a human RPE65 sequence, e.g., a sequence encoding a human RPE65 protein, or an amino acid sequence having at least 75%, 80%, 85%, 90%, 95%, 96%, 97%, 98%, or 99% sequence identity thereto), amaurosis congenita, cone rod dystrophy, choroideremia, vitelliform macular dystrophy, hyperferritinemia-cataract syndrome, optic atrophy, XLR retinoschisis, cytomegalovirus retinitis, achromatopsia, Leber hereditary optical neuropathy, keratitis, uveitis, Grave's opthalmolopathy, diabetic retinopathy, or diabetic macular edema.

In some embodiments, a disease, disorder, or condition (e.g., as described herein) is treated by intravitreal administration of an Anelloviridae family vector as described herein. In some embodiments, a disease, disorder, or condition (e.g., as described herein) is treated by subretinal administration of an Anelloviridae family vector as described herein. In some embodiments, a disease, disorder, or condition (e.g., as described herein) is treated by suprachoroidal administration of an Anelloviridae family vector as described herein.

### VII. Methods of Production

### Producing the Genetic Element

Methods of making the genetic element of the anellovector are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

In some embodiments, the genetic element may be designed using computer-aided design tools. The anellovector may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the anellovector. The segments or ORFs may be assembled into the anellovector, e.g., in vitro recombination or unique restriction sites at 5′ and 3′ ends to enable ligation.

The genetic element can alternatively be synthesized with a design algorithm that parses the anellovector into oligo-length fragments, creating optimal design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element or segment of the genetic element may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO 02088382, WO 03020968, WO 0303 1947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

In some embodiments, factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, this is done both in context of larger regions of the anellovector genome (e.g., inserting effectors into a specific site in the genome, or replacing viral ORFs with effectors).

In another example, when supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

### In Vitro Circularization

In some instances, the genetic element to be packaged into a proteinaceous exterior is a single stranded circular DNA. The genetic element may, in some instances, be introduced into a host cell in a form other than a single stranded circular DNA. For example, the genetic element may be introduced into the host cell as a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or Tral, e.g., as described in Wawrzyniak et al. 2017, Front. Microbiol. 8: 2353; incorporated herein by reference with respect to the listed enzymes). In some embodiments, the double-stranded circular DNA is produced by in vitro circularization, e.g., as described in Example 35 of PCT Publication No. WO 2020/123816, incorporated by reference herein in its entirety. Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Producing the Anellovector

The genetic elements and vectors comprising the genetic elements prepared as described herein can be used in a variety of ways to express the anellovector in appropriate host cells. In some embodiments, the genetic element and vectors comprising the genetic element are transfected in appropriate host cells and the resulting RNA may direct the expression of the anellovector gene products, e.g., non-pathogenic protein and protein binding sequence, at high levels. Host cell systems which provide for high levels of expression include continuous cell lines that supply viral functions, such as cell lines superinfected with APV or MPV, respectively, cell lines engineered to complement APV or MPV functions, etc.

In some embodiments, the anellovector is produced as described in any of Examples 15 or 16. In some embodiments, the anellovector is produced as described in any of Examples 1, 2, 5, 6, or 15-17 of PCT Publication No. WO 2020/123816, incorporated by reference herein in its entirety.

In some embodiments, the anellovector is cultivated in continuous animal cell lines in vitro. According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines susceptible to infection with anellovectors include, but are not limited to cell lines of human or primate origin, such as human or primate kidney carcinoma cell lines.

In some embodiments, the genetic elements and vectors comprising the genetic elements are transfected into cell lines that express a viral polymerase protein in order to achieve expression of the anellovector. To this end, transformed cell lines that express an anellovector polymerase protein may be utilized as appropriate host cells. Host cells may be similarly engineered to provide other viral functions or additional functions.

To prepare the anellovector disclosed herein, a genetic element or vector comprising the genetic element disclosed herein may be used to transfect cells which provide anellovector proteins and functions required for replication and production. Alternatively, cells may be transfected with helper virus before, during, or after transfection by the genetic element or vector comprising the genetic element disclosed herein. In some embodiments, a helper virus may be useful to complement production of an incomplete viral particle. The helper virus may have a conditional growth defect, such as host range restriction or temperature sensitivity, which allows the subsequent selection of transfectant viruses. In some embodiments, a helper virus may provide one or more replication proteins utilized by the host cells to achieve expression of the anellovector. In some embodiments, the host cells may be transfected with vectors encoding viral proteins such as the one or more replication proteins. In some embodiments, a helper virus comprises an antiviral sensitivity.

The genetic element or vector comprising the genetic element disclosed herein can be replicated and produced into anellovector particles by any number of techniques known in the art, as described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

The production of anellovector-containing cell cultures according to the present invention can be carried out in different scales, such as in flasks, roller bottles or bioreactors. The media used for the cultivation of the cells to be infected are known to the skilled person and can generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

The purification and isolation of anellovectors can be performed according to methods known by the skilled person in virus production and is described for example by Rinaldi, et al., DNA Vaccines: Methods and Protocols (Methods in Molecular Biology), 3rd ed. 2014, Humana Press.

In one aspect, the present invention includes a method for the in vitro replication and propagation of the anellovector as described herein, which may comprise the following steps: (a) transfecting a linearized genetic element into a cell line sensitive to anellovector infection; (b) harvesting the cells and isolating cells showing the presence of the genetic element; (c) culturing the cells obtained in step (b) for at least three days, such as at least one week or longer, depending on experimental conditions and gene expression; and (d) harvesting the cells of step (c).

In some embodiments, an anellovector may be introduced to a host cell line grown to a high cell density. In some embodiments, the anellovector may be harvested and/or purified by separation of solutes based on biophysical properties, e.g., ion exchange chromatography or tangential flow filtration, prior to formulation with a pharmaceutical excipient.

### VIII. Administration/Delivery

The composition (e.g., a pharmaceutical composition comprising an anellovector or anelloVLP as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector or anelloVLP to a subject. The method includes administering a pharmaceutical composition comprising an anellovector or anelloVLP as described herein to the subject. In some embodiments, the administered anellovector or anelloVLP replicates in the subject (e.g., becomes a part of the virome of the subject).

The pharmaceutical composition may include wild-type or native viral elements and/or modified viral elements. The anellovector may include one or more of the sequences (e.g., nucleic acid sequences or nucleic acid sequences encoding amino acid sequences thereof) in any one of Tables N1-N32 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any one of Tables N1-N32. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more of the sequences in any one of Tables N1-N32. The anellovector may comprise a nucleic acid molecule encoding an amino acid sequence with at least about 60%, 65%, 70%, 75%, 80%, 85%, 90% 95%, 96%, 97%, 98% and 99% sequence identity to any one of the amino acid sequences in any one of Tables A1-A32. The anellovector may comprise a polypeptide comprising an amino acid sequence with at least about 60%, 65%, 70%, 75%, 80%, 85%, 90% 95%, 96%, 97%, 98% and 99% sequence identity to any one of the amino acid sequences in any one of Tables A1-A32. The anellovector may include one or more of the sequences in any one of Tables A1-A32 or N1-N32, or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any one of Tables N1-N32.

In some embodiments, the anellovector or anelloVLP is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector or anelloVLP is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector or anelloVLP inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an anellovector or anelloVLP described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector or anelloVLP is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector or anelloVLP is sufficient to compete with chronic or acute viral infection. In certain embodiments, the anellovector or anelloVLP may be administered prophylactically to protect from viral infections (e.g. a provirotic). In some embodiments, the anellovector or anelloVLP is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

### Ocular Delivery

In some embodiments, a composition (e.g., an Anelloviridae family vector (e.g., anellovector) or pharmaceutical composition) or method described herein involves an ocular delivery system, such as the Orbit Subretinal Delivery System. Briefly, such a delivery system may comprise a cannula to be inserted into the eye for delivering the Anelloviridae family vector (e.g., anellovector) into the eye, a device body for delivering saline solution or Anelloviridae family vector (e.g., anellovector) to the cannula, a first line for delivering the saline solution to the device body, and a second line for delivering the Anelloviridae family vector (e.g., anellovector) to the device body. More particularly, in some embodiments, the delivery system is provided as three “sets”. The first set is a subretinal injection device set comes with a subretinal injection device, which comprises a cannula tip/needle, a needle advancement knob, a subretinal injection device body (with a magnet), a dose line luer, and a BSS line luer. The system can also comprise with a magnetic pad and an ophthalmic marker. The magnet provides stabilization during injection. The second set (which can be referred to as the tubing set) includes tubing assembly, a BSS syringe, two syringe snap collars, and a CPC adapter. The third set (which can be referred to as the dosing set) comprises a dose syringe and a tubing clamp.

For the subretinal injection device, the internal needle is connected to the needle advancement knob, which is connected to the subretinal injection device body. This has two lines, each attaching to either the BSS line luer or the dose line luer.

Accordingly, in some embodiments, a composition (e.g., an Anelloviridae family vector (e.g., anellovector) or pharmaceutical composition) described herein is situated in an ocular delivery system. The ocular delivery system may comprise, for example:
a) a cannula comprising a first end and a second end, wherein the first end of the cannula optionally comprises a needle,
b) a device body which optionally comprises a magnet, wherein the second end of the cannula is operably connected to the device body,
c) needle advancement knob situated on the device body, wherein the needle advancement knob can be adjusted by a user to advance the needle to extend beyond the first end of the cannula, e.g., extend into the subretinal space;
d) a first line operably attached to the connected to device body (e.g., attached with a luer), wherein the first line may be used to deliver a wash solution, e.g., a saline solution, e.g., BSS, wherein optionally the first line is operably connected to a first syringe (e.g., connected using a syringe snap collar);
e) a second line operably attached to the connected to device body (e.g., attached with a luer), wherein the second line may be used to deliver a composition (e.g., an Anelloviridae family vector (e.g., anellovector) or pharmaceutical composition) described herein, wherein optionally the second line is operably connected to a second syringe (e.g., connected using a syringe snap collar).

In some embodiments, the ocular delivery system is part of a kit. The kit may further comprise one or both of a magnetic pad and an ophthalmic marker.

In some embodiments, a method described herein comprises administering a composition described herein (e.g., an Anelloviridae family vector (e.g., anellovector) or pharmaceutical composition) using an ocular delivery system, e.g., the ocular delivery system described above. In some embodiments, the method comprises surgically preparing the eye for administration of the composition, e.g., by exposing the sclera (e.g., by conjunctival peritomy), optionally transferring ink to the sclera to create a suturing template, creating a suture loop, and creating a sclerotomy. The cannula may be inserted into the sclerotomy. The ocular delivery system may be placed. For instance, the magnetic pad may be placed on the subject's forehead, and the device body may be placed on the magnetic pad, e.g., in the same meridian as the sclerotomy. The first end of the cannula may be positioned directly above the opposite edge of the cornea. Cannulation may be performed. For instance, the suture loops may be lifted and the cannula may be passed through the suture loops. The device body may be slid toward the eye. The cannula may be inserted into the sclerotomy. The needle may be advanced into the subretinal space using the needle advancement knob. A saline solution (e.g., BSS) may be administered, e.g., using the syringe connected to the first line. The saline solution may form a visible bleb. The Anelloviridae family vector (e.g., anellovector) or pharmaceutical composition may be administered, e.g., using the syringe connected to the second line. The Anelloviridae family vector (e.g., anellovector) or pharmaceutical composition may be released into the bleb. The needle may then be retracted. The ocular delivery system may be removed from the eye.

The delivery method may also comprise one or more of the following steps.

The BSS syringe of the tubing set is attached to the delivery system via the BSS line of the subretinal injection device. A plunger is inserted into the dose syringe, followed by attachment of a sterile needle to the dose syringe.

A plunger is also inserted into the dose syringe, and a sterile needle is attached to the dose syringe. This needle is then inserted into the vial and is used to aspirate the subretinal infusate into the syringe. The needle is then removed.

The tab is then rotated into the latched position in order to prime the dose line. The dose syringe is attached to the dose line of the Subretinal injection device. The dose syringe plunger is then advanced slowly until it reaches a hard stop and a tactile click is reached. This primes the dose line and the dose syringe assures the correct subretinal dose volume is ready for injection into the subretinal area.

If using pneumatic injection, the plunger is to be rotated counterclockwise to remove the threaded rod from the BSS syringe and leave the seal in the BSS syringe. The tubing is to be inserted in the open barrel of the BSS syringe and secured in place by sliding the syringe snap collar over both components. The tubing assembly is then attached to the pneumatic source of choice (e.g., a vitrectomy machine) using the CPC adaptor, if necessary. The viscous fluid control injection pressure is to be set to 36 psi.

The provided tubing claims supplied with the subretinal delivery system are only to be used with an alternate dose syringe (not supplied in the set) that is validated for use with the Orbit SDS. The alternate syringe's labeling must indicate that it is validated for use with the Orbit SDS and include instructions for use with the Orbit SDS. If using an alternate dose syringe, the tubing clamp is placed on the dose line following priming to prevent potential backflow into the alternate dose syringe during BSS syringe use. Immediately before injecting the infusate, the tubing clamp is to be removed.

### Exemplary Surgical Steps:

The site it prepared by inserting the lid speculum, and inserting a valved port for the chandelier. The eye is rotated inferonasally to expose the superotemporal quadrant, and a conjunctival peritomy is performed to expose the sclera. A cannulation path that does not interfere with identified vortex veins or long posterior ciliary neurovascular bundles is selected. Ink is applied to the tips of the ophthalmic marker with the limbus, and gently press it against the sclera to transfer the ink. After drying the scleral surface, the marker is aligned with the limbus and is gently pressed against the sclera to transfer ink, and thereby creating the suturing template (about 10 ink dots). The suture loop is created. A sclerotomy is performed.

### Exemplary Device Placement:

The adhesive backing is removed from the magnetic pad, and the pad is placed over the sterile fenestrated drape, on top of the patient's forehead. The primed subretinal injection device body on top of the magnetic pad is placed in the same meridian as the sclerotomy. The distal tip of the subretinal injection cannula is positioned directly above the opposite edge of the cornea to ensure sufficient slack for advancement. The needle is advanced and the flow of BSS or BSS PLUS is checked. The needle is fully retracted.

### Exemplary Cannulation:

Using smooth forceps, the flexible cannula is grasped, approximately 10 mm from the distal tip. Using toothed forceps on the eye to help with insertion, the suture loops are lifted and then the posterior lip of the sclerotomy is grasped. The cannula is passed through the suture loops. Prior to insertion, the subretinal injection device body is slid toward the eye to provide additional slack and maintain a tangential path to the eye's curvature. While grapsing the center of the posterior lip of the sclerotomy and pulling away from the eye, the flexible cannula is inserted into the sclerotomy. The eye is rotated back to the neutral axis.

### Exemplary Method for Using Clearside Biomedical SCS Microinjector:

In some embodiments, a composition (e.g., an Anelloviridae family vector (e.g., anellovector) or pharmaceutical composition) or method described herein involves an ocular delivery system, such as an SCS microinjector. Briefly, such a delivery system may comprise a needle sized appropriately to deliver an Anelloviridae family vector (e.g., anellovector) to the suprachoroidal space, a chamber to contain the Anelloviridae family vector (e.g., anellovector), and a plunger to administer the Anelloviridae family vector (e.g., anellovector). In some embodiments, the microinjector is comprised of a needle of various lengths (needle length is printed on the needle—either 900 um or 1100 um). The needle is a 30 gauge needle. The needle is connected to a conjunctiva compressing hub, which is connected to a chamber (e.g., a barrel), which has a 100 uL capacity and has indicators in increments of 25 uL. The barrel is connected to a plunger and plunger handle to inject the drug. The microinjector also comes with a needle safety cap with integrated fixed length calipers of 4.5 mm.

The Clearside SCS microinjector is designed for suprachoroidal drug delivery.

Accordingly, in some embodiments, a composition (e.g., an Anelloviridae family vector (e.g., anellovector) or pharmaceutical composition) described herein is situated in an ocular delivery system.

The ocular delivery system may comprise:
a) a needle, e.g., a 30 gauge needle, wherein optionally the needle is 800-1200 um (e.g., about 900 um or 1100 um in length);
b) optionally, a conjunctiva compressing hub connected to the needle;
c) a chamber connected to one or both of the conjunctiva compressing hub and the needle; and
d) optionally, a plunger connected to the chamber.

In some embodiments, the ocular delivery system is part of a kit. The kit may further comprise, one or both of a needle safety cap and calipers.

In some embodiments, a method described herein comprises administering a composition described herein (e.g., an Anelloviridae family vector (e.g., anellovector) or pharmaceutical composition) using an ocular delivery system, e.g., the ocular delivery system described above. In some embodiments, the method comprises inserting the needle into the suprachoroidal space and administering the Anelloviridae family vector (e.g., anellovector) or pharmaceutical composition into the suprachoroidal space.

In one aspect, the invention features a method of delivering an Anelloviridae family vector (e.g., anellovector) to a subject, e.g., to an eye of a subject (e.g., to a photoreceptor, retina, posterior eye cup (PEC), retinal ganglion, optic nerve, optic nerve head, subretinal space, intravitreal space, or retinal pigmented epithelium (RPE) of the subject). The method includes administering a pharmaceutical composition comprising an Anelloviridae family vector (e.g., anellovector) as described herein to the subject, e.g., to an eye of a subject (e.g., to a photoreceptor, retina, posterior eye cup (PEC), retinal ganglion, optic nerve, optic nerve head, subretinal space, intravitreal space, or retinal pigmented epithelium (RPE) of the subject). In some embodiments, the administered Anelloviridae family vector (e.g., anellovector) replicates in the subject (e.g., becomes a part of the virome of the subject).

In some embodiments, the method of delivering an Anelloviridae family vector (e.g., anellovector) to a subject comprises contacting the Anelloviridae family vector (e.g., anellovector) to any suitable ocular cell. Ocular cells associated with age-related macular degeneration include, but are not limited to, cells of neural origin, cells of all layers of the retina, especially retinal pigment epithelial cells, glial cells, and pericytes. Other ocular cells that can be contacted as a result of the inventive method include, for example, endothelial cells, iris epithelial cells, corneal cells, ciliary epithelial cells, Mueller cells, astrocytes, muscle cells surrounding and attached to the eye (e.g., cells of the lateral rectus muscle), fibroblasts (e.g., fibroblasts associated with the episclera), orbital fat cells, cells of the sclera and episclera, connective tissue cells, muscle cells, and cells of the trabecular meshwork. Other cells linked to various ocular-related diseases include, for example, fibroblasts and vascular endothelial cells.

Generally, the vector can be delivered in the form of a suspension injected intraocularly (subretinally) under direct observation using an operating microscope. This procedure may involve vitrectomy followed by injection of vector suspension using a fine cannula through one or more small retinotomies into the subretinal space.

Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of vector, and (3) its controlled removal reduces the possibility of retinal tears and unplanned retinal detachment.

In some embodiments, the vector is directly injected into the subretinal space outside the central retina, by utilizing a cannula of the appropriate bore size (e.g. 27-45 gauge), thus creating a bleb in the subretinal space. In other embodiments, the subretinal injection of vector suspension is preceded by subretinal injection of a small volume (e.g. about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of vector suspension to the subretinal space (by defining the plane of injection prior to vector delivery), and minimize possible vector administration into the choroid and the possibility of vector injection or reflux into the vitreous cavity. In some embodiments, this initial fluid bleb can be further injected with fluids comprising one or more vector suspensions and/or one or more additional therapeutic agents by administration of these fluids directly to the intial fluid bleb with either the same or additional fine bore cannulas.

Intraocular administration of the vector suspension and/or the initial small volume of fluid can be performed using a fine bore cannula (e.g. 21-A5 gauge) attached to a syringe. In some embodiments, the plunger of this syringe may be driven by a mechanised device, such as by depression of a foot pedal. The fine bore cannula is advanced through the sclerotomy, across the vitreous cavity and into the retina at a site pre-determined in each subject according to the area of retina to be targeted (but outside the central retina). Under direct visualisation the vector suspension is injected mechanically under the neurosensory retina causing a localised retinal detachment with a self-sealing non-expanding retinotomy. As noted above, the vector can be either directly injected into the subretinal space creating a bleb outside the central retina or the vector can be injected into an initial bleb outside the central retina, causing it to expand (and expanding the area of retinal detachment). In some embodiments, the injection of vector suspension is followed by injection of another fluid into the bleb.

Without wishing to be bound by theory, the rate and location of the subretinal injection(s) can result in localized shear forces that can damage the macula, fovea and/or underlying RPE cells. The subretinal injections may be performed at a rate that minimizes or avoids shear forces. In some embodiments, the vector is injected over about 15-17 minutes. In some embodiments, the vector is injected over about 17-20 minutes. In some embodiments, the vector is injected over about 20-22 minutes. In some embodiments, the vector is injected at a rate of about 35 to about 65 μl/ml. In some embodiments, the vector is injected at a rate of about 35 μl/ml. In some embodiments, the vector is injected at a rate of about 40 μl/ml. In some embodiments, the vector is injected at a rate of about 45 μl/ml. In some embodiments, the vector is injected at a rate of about 50 μl/ml. In some embodiments, the vector is injected at a rate of about 55 μl/ml. In some embodiments, the vector is injected at a rate of about 60 μl/ml. In some embodiments, the vector is injected at a rate of about 65 μl/ml. One of ordinary skill in the art would recognize that the rate and time of injection of the bleb may be directed by, for example, the volume of the vector or size of the bleb necessary to create sufficient retinal detachment to access the cells of central retina, the size of the cannula used to deliver the vector, and the ability to safely maintain the position of the canula of the invention.

One or multiple (e.g. 2, 3, or more) blebs can be created. Generally, the total volume of bleb or blebs created by the methods and systems of the invention can not exceed the fluid volume of the eye, for example about 4 ml in a typical human subject. The total volume of each individual bleb is preferably at least about 0.3 ml, and more preferably at least about 0.5 ml in order to facilitate a retinal detachment of sufficient size to expose the cell types of the central retina and create a bleb of sufficient dependency for optimal manipulation. One of ordinary skill in the art will appreciate that in creating the bleb according to the methods and systems of the invention that the appropriate intraocular pressure must be maintained in order to avoid damage to the ocular structures. The size of each individual bleb may be, for example, about 0.5 to about 1.2 ml, about 0.8 to about 1.2 ml, about 0.9 to about 1.2 ml, about 0.9 to about 1.0 ml, about 1.0 to about 2.0 ml, about 1.0 to about 3.0 ml. Thus, in one example, to inject a total of 3 ml of vector suspension, 3 blebs of about 1 ml each can be established. The total volume of all blebs in combination may be, for example, about 0.5 to about 3.0 ml, about 0.8 to about 3.0 ml, about 0.9 to about 3.0 ml, about 1.0 to about 3.0 ml, about 0.5 to about 1.5 ml, about 0.5 to about 1.2 ml, about 0.9 to about 3.0 ml, about 0.9 to about 2.0 ml, about 0.9 to about 1.0 ml.

In order to safely and efficiently transduce areas of target retina (e.g. the central retina) outside the edge of the original location of the bleb, the bleb may be manipulated to reposition the bleb to the target area for transduction. Manipulation of the bleb can occur by the dependency of the bleb that is created by the volume of the bleb, repositioning of the eye containing the bleb, repositioning of the head of the human with an eye or eyes containing one or more blebs, and/or by means of a fluid-air exchange. This is particularly relevant to the central retina since this area typically resists detachment by subretinal injection. In some embodiments fluid-air exchange is utilized to reposition the bleb; fluid from the infusion cannula is temporarily replaced by air, e.g. from blowing air onto the surface of the retina. As the volume of the air displaces vitreous cavity fluid from the surface of the retina, the fluid in the vitreous cavity may flow out of a cannula. The temporary lack of pressure from the vitreous cavity fluid causes the bleb to move and gravitate to a dependent part of the eye. By positioning the eye globe appropriately, the bleb of subretinal vector is manipulated to involve adjacent areas (e.g. the macula and/or fovea). In some cases, the mass of the bleb is sufficient to cause it to gravitate, even without use of the fluid-air exchange. Movement of the bleb to the desired location may further be facilitated by altering the position of the subject's head, so as to allow the bleb to gravitate to the desired location in the eye. Once the desired configuration of the bleb is achieved, fluid is returned to the vitreous cavity. The fluid is an appropriate fluid, e.g., fresh saline. Generally, the subretinal vector may be left in situ without retinopexy to the retinotomy and without intraocular tamponade, and the retina will spontaneously reattach within about 48 hours.

The composition is administered directly to the eye of a mammal, such as, for example, a mouse, a rat, a non-human primate, or a human. Any administration route is appropriate so long as the composition contacts an appropriate ocular cell. The composition can be appropriately formulated and administered in the form of an injection, eye lotion, ointment, implant, and the like. The composition can be administered, for example, topically, intracamerally, subconjunctivally, intraocularly, retrobulbarly, periocularly (e.g., subtenon delivery), subretinally, or suprachoroidally. Topical formulations are well known in the art. Patches, corneal shields (see, e.g., U.S. Pat. No. 5,185,152), ophthalmic solutions (see, e.g., U.S. Pat. No. 5,710,182), and ointments also are known in the art and can be used in the context of the inventive method. The composition also can be administered non-invasively using a needleless injection device, such as the Biojector 2000 Needle-Free Injection Management SystemT^{M} available from Bioject Medical Technologies Inc. (Tigard, Oreg.).

Alternatively, the composition can be administered using invasive procedures, such as, for instance, intravitreal injection or subretinal injection, optionally preceded by a vitrectomy, or periocular (e.g., subtenon) delivery. The composition can be injected into different compartments of the eye, e.g., the vitreal cavity or anterior chamber. Preferably, the composition is administered intravitreally, most preferably by intravitreal injection.

In some embodiments, the composition may be administered using an ocular delivery system comprising the use of a microneedle (U.S. Pat. No. 8,808,225, incorporated herein in its entirety).

In some embodiments, a method of treating a human subject diagnosed with a disease or disorder of the eye (e.g., nAMD, wet AMD, dry AMD, retinal vein occlusion (RVO), diabetic macular edema (DME), or diabetic retinopathy (DR)), comprises delivering to the retina or posterior eye cup (PEC) of said human subject a therapeutically effective amount of anti-hVEGF antigen-binding fragment, by administering to the intravitreal space, suprachoroidal space, subretinal space, or outer surface of the sclera in the eye of said human subject (e.g., by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with nAMD, dry AMD, retinal vein occlusion (RVO), diabetic macular edema (DME), or diabetic retinopathy (DR) (in particular, wet AMD), comprising delivering to the retina or posterior eye cup (PEC) of said human subject a therapeutically effective amount of anti-hVEGF antigen-binding fragment, by the use of a suprachoroidal drug delivery device such as a microinjector. In a specific aspect, described herein are methods of treating a human subject diagnosed with neovascular age-related macular degeneration (nAMD), dry AMD, retinal vein occlusion (RVO), diabetic macular edema (DME), or diabetic retinopathy (DR) (in particular, wet AMD), comprising delivering to the retina or posterior eye cup (PEC) of said human subject a therapeutically effective amount of anti-hVEGF antigen-binding fragment, wherein the human subject has a Best-Corrected Visual Acuity (BCVA) that is ≤20/20 and ≥20/400.

In some embodiments, a disease, disorder, or condition (e.g., as described herein) is treated by intravitreal administration of an Anelloviridae family vector as described herein. In some embodiments, a disease, disorder, or condition (e.g., as described herein) is treated by subretinal administration of an Anelloviridae family vector as described herein. In some embodiments, a disease, disorder, or condition (e.g., as described herein) is treated by suprachoroidal administration of an Anelloviridae family vector as described herein.

### Redosing

The anellovectors or anelloVLPs described herein can, in some instances, be used as a delivery vehicle that can be administered in multiple doses (e.g., doses administered separately). While not wishing to be bound by theory, in some embodiments, an anellovector or anelloVLP (e.g., as described herein) induces a relatively low immune response (as measured, for example, as 50% GMT values), e.g., allowing for repeated dosing of a subject with one or more anellovectors or anelloVLPs (e.g., multiple doses of the same anellovector or anelloVLP or different anellovectors or anelloVLPs). In an aspect, the invention provides a method of delivering an effector, comprising administering to a subject a first plurality of anellovectors or anelloVLPs and then a second plurality of anellovectors or anelloVLPs. In some embodiments, the second plurality of anellovectors or anelloVLPs comprise the same proteinaceous exterior as the anellovectors or anelloVLPs of the first plurality. In another aspect, the invention provides a method of selecting a subject (e.g., a human subject) to receive an effector, wherein the subject previously received, or was identified as having received, a first plurality of anellovectors or anelloVLPs comprising a genetic element encoding an effector, in which the method involves selecting the subject to receive a second plurality of anellovectors or anelloVLPs comprising a genetic element encoding an effector (e.g., the same effector as that encoded by the genetic element of the first plurality of anellovectors or anelloVLPs, or a different effector as that encoded by the genetic element of the first plurality of anellovectors or anelloVLPs). In another aspect, the invention provides a method of identifying a subject (e.g., a human subject) as suitable to receive a second plurality of anellovectors or anelloVLPs, the method comprising identifying the subject has having previously received a first plurality of anellovectors or anelloVLPs comprising a genetic element encoding an effector, wherein the subject being identified as having received the first plurality of anellovectors or anelloVLPs is indicative that the subject is suitable to receive the second plurality of anellovectors or anelloVLPs.

In some embodiments, the second plurality of anellovectors or anelloVLPs comprises a proteinaceous exterior with at least one surface epitope in common with the anellovectors or anelloVLPs of the first plurality of anellovectors or anelloVLPs. In some embodiments, the anellovectors or anelloVLPs of the first plurality and the anellovectors or anelloVLPs of the second plurality carry genetic elements encoding the same effector. In some embodiments, the anellovectors or anelloVLPs of the first plurality and the anellovectors or anelloVLPs of the second plurality carry genetic elements encoding different effectors.

In some embodiments, the second plurality comprises about the same quantity and/or concentration of anellovectors or anelloVLPs as the first plurality (e.g., when normalized to the body mass of the subject at the time of administration), e.g., the second plurality comprises 90-110%, e.g., 95-105% of the number of anellovectors or anelloVLPs in the first plurality when normalized to body mass of the subject at the time of administration. In some embodiments, wherein the first plurality comprises a greater dosage of anellovectors or anelloVLPs than the second plurality, e.g., wherein the first plurality comprises a greater quantity and/or concentration of anellovectors or anelloVLPs relative to the second plurality. In some embodiments, wherein the first plurality comprises a lower dosage of anellovectors or anelloVLPs than the second plurality, e.g., wherein the first plurality comprises a lower quantity and/or concentration of anellovectors or anelloVLPs relative to the second plurality.

In some embodiments, the subject is evaluated between the administration of the first and second pluralities of anellovectors or anelloVLPs, e.g., for the presence (e.g., persistence) of anellovectors or anelloVLPs from the first plurality, or progeny thereof. In some embodiments, the subject is administered the second plurality of anellovectors or anelloVLPs if the presence of anellovectors or anelloVLPs from the first plurality, or the progeny thereof, are not detected.

In some embodiments, the second plurality is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality to the subject. In some embodiments, the second plurality is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality to the subject. In some embodiments, the method comprises administering a repeated dose of anellovectors or anelloVLPs over the course of at least 1, 2, 3, 4, or 5 years.

In some embodiments, the method further comprises assessing, after administration of the first plurality and before administration of the second plurality, one or more of:
a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);
b) the level or activity of the anellovector or anelloVLP of the first plurality in the subject (e.g., by detecting the level of the VP1 of the anellovector or anelloVLP);
c) the presence, severity, progression, or a sign or symptom of a disease in the subject that the anellovector or anelloVLP was administered to treat; and/or
d) the presence or level of an immune response, e.g., neutralizing antibodies, against an anellovector or anelloVLP.

In some embodiments, the method further comprises administering to the subject a third, fourth, fifth, and/or further plurality of anellovectors or anelloVLPs, e.g., as described herein.

In some embodiments, the first plurality and the second plurality are administered via the same route of administration, e.g., intravenous administration. In some embodiments, the first plurality and the second plurality are administered via different routes of administration. In some embodiments, the first and the second pluralities are administered by the same entity (e.g., the same health care provider). In some embodiments, the first and the second pluralities are administered by different entities (e.g., different health care providers).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used. Additional examples can be found, for example, in PCT Publication No. WO 2020/123816, incorporated by reference herein in its entirety.

### EXAMPLES

### Table of Contents

Example 1: Expression of ORF1 constructs for conjugation
Example 2: Conjugation of peptides via NHS click chemistry
Example 3: Conjugation of polypeptides via two-step click chemistry
Example 4: Conjugation of polypeptides via maleimide conjugation
Example 5: Genetic grafting of surface effector to the VLP surface
Example 6: Generation of circular ssDNA transgene for in vitro assembly of Anellovectors
Example 7: Anellovector ORF1 VLP expression in insect or mammalian cells and purification
Example 8: Expression of Ring2 and Ring10 Anellovirus ORF1s in insect cells
Example 9: Expression of CAV capsid protein VP1 in mammalian cells
Example 10: Expression of Ring2 ORF1 and ORF2 in insect cells
Example 11: Disassembly of Ring2 VLPs using urea
Example 12: Dissociation of Anellovirus VLPs and reassembly around nucleic acid cargoes
Example 13: Replication-deficient anellovectors and helper viruses
Example 14: Manufacturing process for replication-competent anellovectors
Example 15: Manufacturing process of replication-deficient anellovectors: recovery and scaling up of production of replication-deficient anellovectors
Example 16: Production of anellovectors using suspension cells: production of anellovectors in cells in suspension.
Example 17: Antibody generation and western blot analysis
Example 18: Construct design, cell culture and protein expression/purification
Example 19: Negative-stained EM data collection and analysis
Example 20: Cryo-EM data collection and data analysis and molecular refinement
Example 21: Circular dichroism spectroscopy
Example 22: Anellovirus particle structure
Example 23: Anellovirus jelly roll domains
Example 24: Anellovirus spike domains
Example 25: Evasion of immune system
Example 26: Grafting of HVR helix motif between Anellovirus ORF1 proteins
Example 27: Production and purification of AnelloVLPs
Example 28: Conjugation of AnelloVLPs
Example 29: VLP conjugation to SARS-CoV-2 RBD peptide
Example 30. Ring19-eGFP Anellovector transduces eye tissue in vivo
Example 31. Expression of GFP protein in human RPE cells after transduction with engineered Ring19 Anellovector
Example 32. Transduction of ocular tissue with varying doses of Ring19-eGFP
Example 33. A Cre recombinase based three-plasmid system for Anelloviridae family vector particle production
Example 34. A Bxb1 recombinase based three-plasmid system for anellovector viral particle production
Example 35. Different promoters can drive transgene expression in the Cre and Bxb1 recombinase systems
Example 36. Enhancing vector production using alternative origins of replication
Example 37. A recombinase based two-plasmid system for Anelloviridae family vector particle production
Example 38. Isolation of cssDNA using nicking enzymes and exonuclease digest
Example 39. Isolation of cssDNA from a Ring19-eGFP minicircle using nicking enzymes and exonuclease digest
Example 40. Isolation of cssDNA from a Ring2-eGFP minicircle using nicking enzymes and exonuclease digest
Example 41. Production of minicircles in bacterial cells using a recombinase system
Example 42. Use of minicircles for production of Anelloviridae family vector particles
Example 43. Removing homology between the vector and SRR plasmids reduces production of wild-type (WT) Anelloviridae family viral genomes
Example 44. Self-replicating rescue (SRR) plasmid configurations
Example 45. Knocking out expressing of ORF1 in the self-replicating rescue (SRR) plasmid does not affect expression of other ORF proteins and splice variants
Example 46. The Ring19 genetic element is successfully packaged by other Ring ORF1 molecules
Example 47. In vivo transduction of cross-packaged R2 ORF1 and Ring19 genetic element Anellovector

### Example 1. Expression of ORF1 Constructs for Conjugation

In nature, Anelloviruses encapsidate circularized negative sense (ns) single stranded (ss) DNA genomes in part through interactions between the genome and the N-terminal arginine rich motif (ARM). UV absorption evidence suggests recombinant ORF1 containing the ARM forms virus like particles (VLPs) that are bound to nucleic acids, presumably non-specific host cell DNA fragments. To remove these potentially undesired host cell impurities, ORF1 constructs are generated in which the ARM is deleted (delARM ORF1).

Expression of Arginine-Rich Region Deletion (delARM) ORF1 Constructs

In one example, a delARM mutant was generated for the Ring 10 ORF1 protein, which was then produced in Sf9 insect cells. Without wishing to be bound by theory, this VLP may not contain the same level of host cell nucleic acid impurities as the structural arginine-rich region, also referred to herein as the structural arginine-rich motif (ARM; also referred to herein as the structural arginine-rich region), is removed (referred to herein as “delARM”). Ring 10 delARM ORF1 is expressed with the complete C-terminal region present. During purification, the structural C-terminal domain or a portion thereof is clipped by protease activity resulting in a C-terminal deletion delARM construct.

Anellovirus ORF1 molecules, such as an ORF1 protein in which the ARM has been deleted, harbor a basic residue (lysine) on the particle surface which would be suitable for conjugating surface effectors including peptides, proteins such as antibodies, glycan groups or neuclic acid entities which are covalently bound to N-hydroxysuccinimide (NHS). NHS is a click-chemistry which binds to amino groups such as those located on lysine residues.

### Expression of C-Terminal Deletion ORF1 Constructs

Recombinant ORF1s do not need the C-terminal region to form VLPs. While the biological role of the C-terminal region has yet to be elucidated regarding virus formation or viral infection, we have demonstrated that the C-terminal region can be removed and permit efficient VLP formation. One example is the Ring 2 ORF1 C-terminal truncation at residue 611 generated in 293 cells (e.g., as listed in Table B1-3a). After observing that Ring 10 delARM has the C-terminus cleaved by proteases during the purification process, we explored generating VLPs lacking C-terminal region to make a more uniform product. Without wishing to be bound by theory, it is contemplated that deleting this region for the AnelloVLP surface effector conjugation approach may eliminate immune responses to the C-terminal region (i.e. eliminate anti-drug responses). In addition, generating ORF1 VLPs in mammalian cells (e.g., 293 cells) further suggests these particles could be generated in other eukaryotic cell lines such as stable CHO cell lines to provide alternative production processes.

### Example 2. Conjugation of Peptides Via NHS Click Chemistry

Based on the structural analysis described herein, basic residues (lysine residues) of Ring 10 ORF1 were identified as surface exposed on the VLP viral surface. Several surface effectors can be conjugated to the amine groups of lysine by adding the click-chemistry entity NHS. In one example, the short antigenic region of the malaria CS protein (for example, the NANP repeat region known to be immunogenic in the R21 malaria vaccine currently being evaluated in the clinic) can be synthesized with an NHS moiety. By mixing the NANP-NHS peptide with ORF1 VLPs, we can conjugate the antigenic region of the R21 vaccine candidate to the ORF1 VLP generating a novel malaria particle as a vaccine candidate. Conjugation of other entities to the surface lysines though NHS conjugation could also be generated such as anti-sense oligomers (ASOs) or glycans from other bacteria known to provide an immune response in vaccines (such as, for example, the glycans of Prevnar13).

### Example 3. Conjugation of Polypeptides Via Two-Step Click Chemistry

As discussed in Example 2, surface exposed lysines are available for NHS conjugation. However, not every surface effector may be available for synthetic addition of NHS such as peptides, oligomers or glycans. In one example, larger surface effectors such as proteins (antibody fragments or larger vaccine antigens) could be generated with free cysteine, which can further be conjugated with click-chemistries, for example, by virtue of maleimide-click-chemistry linkers (maleimide being a click chemistry that binds thiols of free cysteine), which in turn can be added to paired chemistries on conjugated to free lysine on the VLP surface. In one example, a free cysteine could be engineered into a polypeptide encoding the C-terminal (immunogenic) portion of the malaria CS protein. This protein could then be conjugated by virtue of its free cysteine to a click chemistry moiety such as azide. The ORF1 VLP could be further conjugated by NHS-DBCO (DBCO being the click-chemistry partner of azide) by virtue of the surface lysines on ORF1. When the VLP-DBCO species is combined with the Azide-Malaria polypeptide species, the DBCO-Azide conjugation would produce a covalently attached Malaria-VLP particle suitable as a malaria vaccine candidate. This two-step conjugation approach could be suitable for other larger surface effectors where direct synthesis with click-chemistry moieties is not available, including antibodies and larger nucleic acid oligomers.

### Example 4. Conjugation of Polypeptides Via Maleimide Conjugation

In Examples 2 or 3 above, conjugation of surface effectors is done through conjugation of NHS moieties to surface lysines. This approach could have the added advantage of adding several surface effectors to the ORF1, depending on how many lysines are surface exposed in a given strain of ORF1. However, specific conjugation to ORF1 may be desired for some surface effectors to generate a more controlled product. In this example, a Ring 10 ORF1 structure (determined according to the structural analysis described herein) is used to define surface exposed regions of the VLP and introduce a free cysteine suitable for maleimide conjugation (maleimide being a click-chemistry that binds thiols in free cysteine residues), for example, at residues in the P2 domain of Ring10, such as Threonine 365. These engineered VLPs, perhaps in combination with deletion of the ARM as described in Example 1, would provide a cleaner, more controlled conjugation. By replacing the NHS species in Examples 2 and 3 with maleimide moieties, the conjugation of the surface effector, such as a maleimide malaria peptide, could be specifically targeted to a particular residue of interest, e.g., at a controlled stoichiometry.

To ensure only the desired target cysteines are available for modification with antigenic peptides or other surface effector molecules, the native cysteine residues of ORF1 (e.g., located at positions: 57, 64, 112, 131, 220, 223, 626) will also be replaced with either serine or alanine residues. When the residue to be mutated is on the surface of the molecule and the sidechain is exposed to solvent, replacing a cysteine with a serine is a conservative alteration as the two residues are isosteric and therefore should not disrupt the 3-dimensional structure of the protein. When the residue to be mutated is buried inside of the structure, alanine is a more conservative alteration as the polar side chain of serine is likely to destabilize the structure.

### Example 5. Genetic Grafting of Surface Effector to the VLP Surface

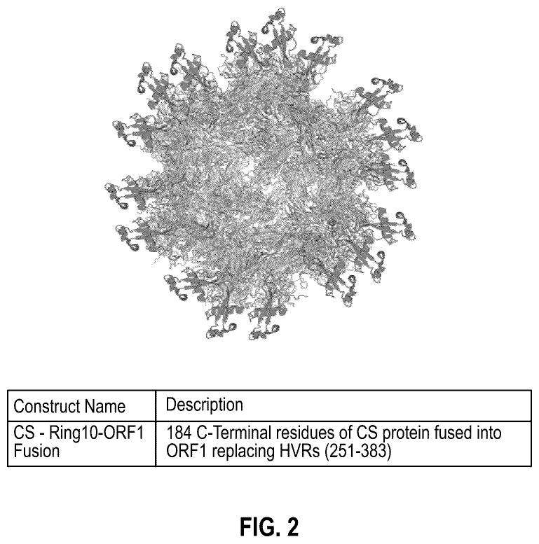

Using a structure determined for Ring 10, the region of the VLP that is surface exposed can be identified, for example, such as an HVR region. In this example, a VLP construct is generated in which a surface effector coding sequence replaces or is inserted into the HVR region of ORF. One example could be the genetic fusion of the malaria CS immunogenic portion into the HVR region of ORF1 VLPs (FIG. 2). In these genetic fusions, the VLP does not require conjugation, but rather a fusion protein comprises an ORF1 portion and a surface effector portion, such that the surface effector portion is displayed on the outer surface of the VLP. The advantage of this approach would be alleviating the need to conjugate the VLP post purification, which may not be an efficient process. Another advantage would be that each copy of the ORF1 molecule would be known to contain one copy of the surface effector. In some iterations it may be advantageous to express ORF1 with and without surface effectors generating mixed VLPs with fewer surface effectors.

Without wishing to be bound by theory, it is contemplated that the truncation of N- and C-termini will be beneficial for anelloVLP formation. Positive charged N-termini without nucleic acid appeared to improve the packaging of anelloVLP, as observed by electron microscopy in anelloviruses. Removal of the N-terminal peptide has been shown to boost anelloVLP titer and provide a more homogeneous morphology. On the other end, although the C-terminal domain does not have any charge tendency, the potential post-translational modification may hamper the maturation of anelloVLPs, as shown by electron microscopy. The removal of the C-terminal domain avoids such maturation process and thereby is expected to stabilize anelloVLP particle morphology. Based on these studies, we have engineered both N- and C-termini to carry different tags for purification, fluorescent tag labeling for biodistribution assay, or other epitopes as shown in Tables B2-4, B2-5, or B2-6. For example, we have inserted an AVI tag on the exterior P2 site for biotinylation or other epitope for vaccine development, e.g. malaria CS protein (see Table B2-4). Thus, a surface effector may be grafted onto the N- and/or C-terminus of an ORF 1 molecule.

In an example, a surface effector and ORF1 fusion protein comprises an amino acid sequence as listed in Table E1 below.

TABLE E1
Exemplary ORF1-surface effector fusion protein amino acid sequences

| Construct Name | Amino Acid Sequence |
|---|---|
| R21 construct | ANPNANPNANPNANPNANPNANPNANPNANPNANPNANPNANPNANPN |
| [184 C-Terminal residues of CS | ANPNANPNANPNKNNQGNGQGHNMPNDPNRNVDENANANSAVKNNNNE |
| protein (CS-Cterm) fused | EPSDKHIKEYLNKIQNSLSTEWSPCSVTCGNGIQVRIKPGSANKPKDE |
| to the Hepatitis B Virus Surface | LDYANDIEKKICKMEKCSSVFNVVNSSIGLIMVLSFLFLNENTTSGFL |
| Antigen (HBsAg)] | GPLLVLQAGFFLLTRILTIPQSLDSWWTSLNFQGGAPTCPGONSQSPT |
| | SNHSPTSCPPICPGYRWMCLRRFIIFLFILLLCLIFLLVLLDYQGMLP |
| | VCPLLPGTSTTGTGPCRTCTIPAQGTSMFPSCCCTKPSDGNCTCIPIP |
| | SSWAFARFLWEWASVRFSWLSLLVPFVQWFAGLSPTVWLSVIWMMWYR |
| | GPSLYNTLSPFLPLLPISFCLWVYI (SEQ ID NO: 2673) |
| R21 construct + C-Terminal | ANPNANPNANPNANPNANPNANPNANPNANPNANPNANPNANPN |
| Cysteine | ANPNANPNANPNANPNKNNQGNGQGHNMPNDPNRNVDENANANS |
| [184 C-Terminal residues of CS | AVKNNNNEEPSDKHIKEYLNKIQNSLSTEWSPCSVTCGNGIQVR |
| protein (CS-Cterm-184) fused | IKPGSANKPKDELDYANDIEKKICKMEKCSSVFNVVNSSIGLIM |
| to the Hepatitis B Virus Surface | VLSFLFLNENTTSGFLGPLLVLQAGFFLLTRILTIPOSLDSWWT |
| Antigen (HBsAg) | SLNFQGGAPTCPGQNSQSPTSNHSPTSCPPICPGYRWMCLRRFI |
| with a Cysteine residue for | IFLFILLLCLIFLLVLLDYQGMLPVCPLLPGTSTTGTGPCRTCT |
| conjugation] | IPAQGTSMFPSCCCTKPSDGNCTCIPIPSSWAFARFLWEWASVR |
| | FSWLSLLVPFVQWFAGLSPTVWLSVIWMMWYRGPSLYNTLSPFL |
| | PLLPISFCLWVYIC (SEQ ID NO: 2674) |
| SB-7062 R21 CS C-Terminus | MKIKTGARILALSALTTMMFSASALAANPNANPNANPNANPNAN |
| [184 C-Terminal residues of CS | PNANPNANPNANPNANPNANPNANPNANPNANPNANPNANPNKN |
| protein | NQGNGQGHNMPNDPNRNVDENANANSAVKNNNNEEPSDKHIKEY |
| for 293 expression] | LNKIQNSLSTEWSPCSVICGNGIQVRIKPGSANKPKDELDYAND |
| | IEKKICKMEKCSSVFNVVNSSIGLIMVLSFLFLNPHHHHHHA |
| | (SEQ ID NO: 2675) |
| SB-7062 R21 CS C-Terminus + | MKIKTGARILALSALTTMMFSASALAANPNANPNANPNANPNAN |
| C-Terminal Cysteine | PNANPNANPNANPNANPNANPNANPNANPNANPNANPNANPNKN |
| [184 C-Terminal residues of CS | NQGNGQGHNMPNDPNRNVDENANANSAVKNNNNEEPSDKHIKEY |
| protein | LNKIQNSLSTEWSPCSVTCGNGIQVRIKPGSANKPKDELDYAND |
| with additional C-Terminal | IEKKICKMEKCSSVFNVVNSSIGLIMVLSFLFLNPHHHHHHAC |
| Cysteine for conjugation] | (SEQ ID NO: 2676) |
| R21 Peptide NANP₍₆ₓ₎ | NPNANPNANPNANPNANPNANPNA(K/Mal)-amide (SEQ |
| [Peptide of N-Terminal | ID NO: 2677) |
| residues of R21 6x(NANP) | |
| with NHS for conjugation] | |
| R21 Peptide NANP-2 | ANPNKNNQGNGQGHNMPNDPNRNVDENANANSAVKNNNNE(K/Mal- |
| [Peptide of second segment of | amide (SEQ ID NO: 2678) |
| R21 with NHS for conjugation] | |
| R21 Peptide Solved Structure | EPSDKHIKEYLNKIONSLSTEWSPSSVTSGNGIQVRIKPGSANK |
| [Structured portion of C- | PKDELDYANDIEKKISKMEKSS(K/Mal)-amide (SEQ ID |
| Terminal segment of R21] | NO: 2679) |
| Ring 10 (Ly1) Wt protein | MPWWYRRRSYNPWRRRNWFRRPRKTIYRRYRRRRRWVRRKPFYK |
| sequence | RKIKRLNIVEWQPKSIRKCRIKGMLCLFQTTEDRLSYNFDMYEE |
| | SIIPEKLPGGGGFSIKNISLYALYQEHIHAHNIFTHTNTDRPLA |
| | RYTGCSLKFYQSKDIDYVVTYSTSLPLRSSMGMYNSMQPSIHLM |
| | QQNKLIVPSKQTQKRRKPYIKKHISPPTQMKSQWYFQHNIANIP |
| | LLMIRTTALTLDNYYIGSRQLSTNVTIHTLNITYIQNRDWGDRN |
| | KTYYCQTLGTQRYFLYGTHSTAQNINDIKLQELIPLINTQDYVQ |
| | GFDWTEKDKHNITTYKEFLTKGAGNPFHAEWITAQNPVIHTANS |
| | PTQIEQIYTASTTTFQNKKLTDLPTPGYIFITPTVSLRYNPYKD |
| | LAERNKCYFVRSKINAHGWDPEQHQELINSDLPQWLLLFGYPDY |
| | IKRTQNFALVDTNYILVDHCPYTNPEKTPFIPLSTSFIEGRSPY |
| | SPSDTHEPDEEDQNRWYPCYQYQQESINSICLSGPGTPKIPKGI |
| | TAEAKVKYSFNFKWGGDLPPMSTITNPTDQPTYVVPNNFNETTS |
| | LONPTTRPEHFLYSFDERRGQLTEKATKRLLKDWETKETSLLST |
| | EYRFAEPTQTQAPQEDPSSEEEEESNLFERLLRORTKOLQLKRR |
| | IIQTLKDLQKLE (SEQ ID NO: 2680) |
| CCN5 CTermCys | MRGTPKTHLLAFSLLCLLSKVRTQLCPTPCTCPWPPPRCPLGVP |
| | LVLDGCGCCRVCARRLGEPCDQLHVCDASQGLVCQPGAGPGGRG |
| | ALCLLAEDDSSCEVNGRLYREGETFQPHCSIRCRCEDGGFTCVP |
| | LCSEDVRLPSWDCPHPRRVEVLGKCCPEWVCGQGGGLGTQPLPA |
| | QGPQFSGLVSSLPPGVPCPEWSTAWGPCSTTCGLGMATRVSNON |
| | RFCRLETQRRLCLSRPCPPSRGRSPONSAFGGGGSGGGGSGGGG |
| | SC (SEQ ID NO: 2681) |
| Aflibercept malE_CTermCys | MKIKTGARILALSALTTMMFSASALASDTGRPFVEMYSEIPEII |
| | HMTEGRELVIPCRVTSPNITVTLKKFPLDTLIPDGKRIIWDSRK |
| | GFIISNATYKEIGLLTCEATVNGHLYKTNYLTHRQTNTIIDVVL |
| | SPSHGIELSVGEKLVLNCTARTELNVGIDENWEYPSSKHQHKKL |
| | VNRDLKTQSGSEMKKFLSTLTIDGVTRSDQGLYTCAASSGLMTK |
| | KNSTFVRVHEKDKTHTCPPCPAPELLGGPSVFLFPPKPKDTLMI |
| | SRTPEVTCVVVDVSHEDPEVKFNWYVDGVEVHNAKTKPREEQYN |
| | STYRVVSVLIVLHQDWLNGKEYKCKVSNKALPAPIEKTISKAKG |
| | QPREPQVYTLPPSRDELTKNQVSLTCLVKGFYPSDIAVEWESNG |
| | QPENNYKTTPPVLDSDGSFFLYSKLTVDKSRWQQGNVFSCSVMH |
| | EALHNHYTQKSLSLSPGGGGSGGGGSGGGGSGGGGSC (SEQ ID |
| | NO: 2682) |
| Aflib_D1-D2 malE_CTermCys | MKIKTGARILALSALTTMMFSASALASDTGRPFVEMYSEIPEII |
| | HMTEGRELVIPCRVISPNITVTLKKFPLDTLIPDGKRIIWDSRK |
| | GFIISNATYKEIGLLTCEATVNGHLYKTNYLTHRQTNTIIDVVL |
| | SPSHGIELSVGEKLVLNCTARTELNVGIDENWEYPSSKHQHKKL |
| | VNRDLKTQSGSEMKKFLSTLTIDGVTRSDQGLYTCAASSGLMTK |
| | KNSTFVRVHEKGGGGSGGGGSGGGGSC (SEQ ID NO: 2683) |
| Ranibizumab HC | MKIKTGARILALSALTTMMFSASALAEVQLVESGGGLVQPGGSL |
| malE_CTermCys | RLSCAASGYDFTHYGMNWVRQAPGKGLEWVGWINTYTGEPTYAA |
| | DFKRRFTFSLDISKSTAYLQMNSLRAEDTAVYYCAKYPYYYGTS |
| | HWYFDVWGQGTLVTVSSASTKGPSVFPLAPSSKSTSGGTAALGC |
| | LVKDYFPEPVTVSWNSGALTSGVHTFPAVLQSSGLYSLSSVVTV |
| | PSSSLGTQTYICNVNHKPSNTKVDKKVEPKSCDKTHLGGGGSGG |
| | GGSGGGGSC (SEQ ID NO: 2684) |
| Bevacizumab HC | MKIKTGARILALSALTTMMFSASALAEVQLVESGGGLVQPGGSL |
| malE_CTermCys | RLSCAASGYTFTNYGMNWVRQAPGKGLEWVGWINTYTGEPTYAA |
| | DFKRRFTFSLDTSKSTAYLOMNSLRAEDTAVYYCAKYPHYYGSS |
| | HWYFDVWGQGTLVTVSSASTKGPSVFPLAPSSKSTSGGTAALGC |
| | LVKDYFPEPVTVSWNSGALTSGVHTFPAVLQSSGLYSLSSVVTV |
| | PSSSLGTQTYICNVNHKPSNTKVDKKVEPKSCDKTHTCPPCPAP |
| | ELLGGPSVFLFPPKPKDTLMISRTPEVTCVVVDVSHEDPEVKFN |
| | WYVDGVEVHNAKTKPREEQYNSTYRVVSVLIVLHQDWLNGKEYK |
| | CKVSNKALPAPIEKTISKAKGQPREPQVYTLPPSREEMTKNQVS |
| | LTCLVKGFYPSDIAVEWESNGQPENNYKTTPPVLDSDGSFFLYS |
| | KLTVDKSRWQQGNVFSCSVMHEALHNHYTQKSLSLSPGKKDELG |
| | GGGSGGGGSGGGGSC (SEQ ID NO: 2685) |
| scFab RaniHC-link50-LC | MKIKTGARILALSALTTMMFSASALAEVQLVESGGGLVQPGGSL |
| malE_CTermCys | RLSCAASGYDFTHYGMNWVRQAPGKGLEWVGWINTYTGEPTYAA |
| | DFKRRFTFSLDTSKSTAYLQMNSLRAEDTAVYYCAKYPYYYGTS |
| | HWYFDVWGQGTLVTVSSASTKGPSVFPLAPSSKSTSGGTAALGC |
| | LVKDYFPEPVTVSWNSGALTSGVHTFPAVLQSSGLYSLSSVVTV |
| | PSSSLGTQTYICNVNHKPSNTKVDKKVEPKSCDKTHLGGSSGSG |
| | SGSTGTSSSGTGTSAGTTGTSASTSGSGSGGGGGSGGGGSAGGD |
| | IQLTQSPSSLSASVGDRVTITCSASQDISNYLNWYQQKPGKAPK |
| | VLIYFTSSLHSGVPSRFSGSGSGTDFTLTISSLQPEDFATYYCQ |
| | QYSTVPWTFGQGTKVEIKRTVAAPSVFIFPPSDEQLKSGTASVV |
| | CLLNNFYPREAKVQWKVDNALQSGNSQESVTEQDSKDSTYSLSS |
| | TLTLSKADYEKHKVYACEVTHQGLSSPVTKSFNRGECGGGGSGG |
| | GGSGGGGSC (SEQ ID NO: 2686) |

### Example 6. Generation of Circular ssDNA Transgene for In Vitro Assembly of Anellovectors

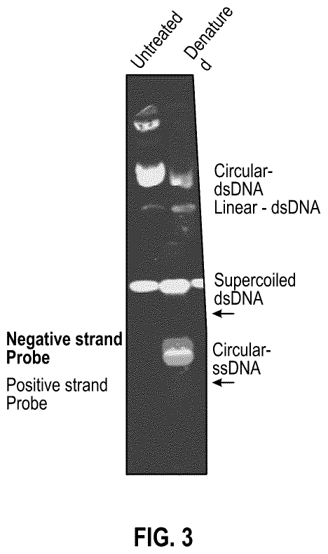

Anelloviruses encapsidate circularized negative sense (ns) single stranded (ss) DNA genomes. Described in this example is a method for generating circularized ns-ssDNA purified in vitro. First, the genome of an Anellovirus (e.g., the genome of strain Ring 2 as described herein) was generated in a plasmid for amplification. Next, the DNA encoding the viral genome was cut from the plasmid and re-ligated to form double-stranded in vitro circularized (IVC) DNA. The IVC DNA was denatured with heat by boiling at 80° C. for 5 minutes in the presence of urea and run on a denaturing gel overnight at 4° C. When the gel is stained with fluorescently-labeled primers from either the positive strand (to bind the negative-sense sequence) or the negative strand (to bind the positive-sense sequence) it was observed that the IVC DNA was separated into positive and negative sense circularized ssDNA (FIG. 3).

The ssDNA can be extracted from the gel using conventional DNA purification kits and used in anellovector in vitro encapsidation screening. Plasmids can also be cloned that encode viral genomes that also harbor reporter genes or therapeutic genes, for example, such that generation and encapsidation of the resulting circularized ssDNA would generate an anellovector that can transduce cells with the reporter or therapeutic gene.

### Example 7. Anellovector ORF1 VLP Expression in Insect or Mammalian Cells and Purification

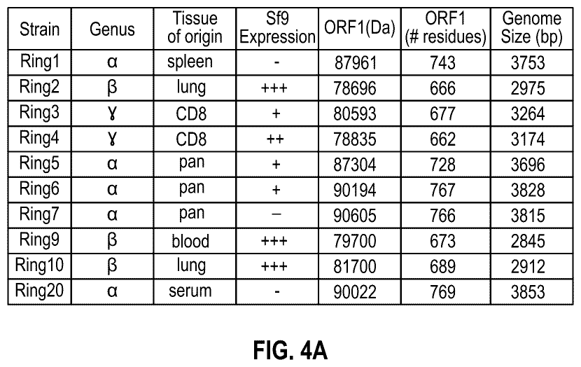

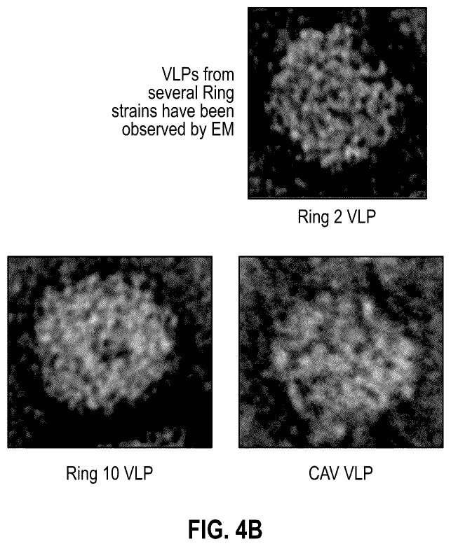

Described in this example are expression and purification protocols for Anellovirus ORF1 recombinant proteins and variations that can be used, for example, in generating anellovector strains in vitro. Several Anellovirus ORF1 strains have been successfully expressed in insect (Sf9) and/or mammalian (293) cell lines (FIG. 4A). As described in Examples 8 and 9 below, several ORF1 proteins from different Anellovirus strains (as well as the related CAV Vpl capsid protein) were shown to have a propensity to self-assemble into VLPs in vitro when observed by electron microscopy (FIG. 4B).

In one example, anellovectors can be produced in vitro by generating expression constructs for certain Anellovirus proteins (e.g., as described herein), such as an Anellovirus ORF1. Such expression constructs can be introduced into cells (e.g., mammalian cells or insect cells, e.g. Sf9 cells) to produce Anellovirus ORF1 proteins capable of forming capsids. Production of the ORF1 proteins can be done in the presence or absence of a suitable Anellovirus ORF2 protein. The expression constructs can also be engineered to attach (e.g., fuse) an affinity tag to the Anellovirus ORF1 protein. The ORF1 proteins can then be purified from the cells.

In some examples, a desired payload nucleic acid molecule (e.g., an engineered Anellovirus genome, a wild-type Anellovirus genome, an oligonucleotide, a single-stranded DNA, a double-stranded DNA, or an RNA, e.g., an mRNA) can be encapsulated by the ORF1 proteins in vitro, under conditions suitable for assembly of the ORF1 proteins into a proteinaceous exterior (e.g., a capsid). Such conditions may include, for example, a solution comprising one or more of: detergents, buffers, and/or reducing agents (e.g., as described herein), as well as incubation at a preselected temperature for a preselected time. This assembly can result in formation of particles (e.g., comprising assembled ORF1 proteins encapsulating the desired payload nucleic acid molecule). Particle formation can be assessed, for example, by co-migration of ssDNA in size exclusion chromatography and/or by detecting DNase-resistant payload nucleic acid molecules (e.g., by PCR), e.g., as described herein. In addition, particles can be assessed for their capacity to transduce target cells with the payload nucleic acid molecule (e.g., by detecting a gene product encoded by the payload nucleic acid molecule, e.g., a reporter).

In addition to exploring the purification and in vitro assembly of ORF1 from different strains, Anellovirus ORF1 fragments or chimeric molecules can be used to improve the efficiency of in vitro assembly. Such fragment or chimeric molecules include, but are not limited to, Anellovirus ORF1 proteins that harbor mutations introduced into the N-terminal Arginine-rich region (also referred to as the ARG arm) to alter the pI of the ARG arm, permitting pH sensitive nucleic acid binding to trigger particle assembly (SEQ ID NOs: 563-565). Anellovirus ORF1 mutations that improve stability include, for example, mutations to the interprotomer contacting beta strands F and G of the canonical structural jellyroll beta-barrel (F and G beta strands) to alter the hydrophobic state of the protomer surface to make capsid formation more thermodynamically favored.

Exemplary chimeric ORF1 proteins include, but are not limited to, Anellovirus ORF1 proteins which have a portion or portions of their sequence replaced with comparable portions from another capsid protein, such as BFDV, CAV capsid protein or Hepatitis E (such as the ARG arm or F and G beta strands of Ring 9 ORF1 replaced with the comparable components from BFDV capsid protein; SEQ ID NOs: 566-567). Chimeric ORF1 proteins may also include ORF1 proteins which have a portion or portions of their sequence replaced with comparable portions of another Anellovirus ORF1 protein (such as structural jellyroll fragments or the C-terminal portion of Ring 2 ORF1 replaced with comparable portions of Ring 9 ORF1; SEQ ID NOs: 568-575).

Proteins will be purified using purification techniques, including but not limited to chelating purification, heparin purification, gradient sedimentation purification and/or SEC purification.

### Example 8. Expression of Ring2 and Ring10 Anellovirus ORF1s in Insect Cells

In this example, DNA sequences encoding Ring 2 ORF1 or Ring 10 ORF1, each fused to an N-terminal HIS₆-tag (SEQ ID NO: 2743) (HIS-ORF1), was codon optimized for insect expression and cloned into the baculovirus expression vector pFASTbac system according to the manufacture's method (ThermoFisher Scientific). Insect cells (Sf9 cells) were infected with Ring HIS-ORF1 baculovirus and the cells were harvested 3-days post-infection by centrifugation. The cells were lysed and the protein was purified using a chelating resin affinity column (HisTrap, GE Healthcare). The resulting material was purified again using a heparin affinity column (Heparin HiTrap, GE Healthcare) and fractions containing ORF1 were analyzed by negative staining electron microscopy. Both Ring 2 ORF1 and Ring 10 ORF1 exhibited an observed propensity to form ˜35 nm virus-like particles (VLPs) in this in vitro setting (FIG. 4B).

### Example 9. Expression of CAV Capsid Protein VP1 in Mammalian Cells

In this example, DNA sequences encoding CAV capsid protein (CAV Vpl), fused to an N-terminal HIS₆—Flag-tag (“HHHHHH” disclosed as SEQ ID NO: 2743) (HIS-Flag-Vp1), and helper protein (Vp2) were codon optimized for mammalian expression and cloned into a mammalian expression vector including a CMV promoter. Mammalian cells (293expi cells) were transfected with CAV Vp1 and Vp2 expression vectors. The cells were harvested 3-days post-infection by centrifugation. The cells were lysed and the lysis was purified using chelation and heparin purification as described in Example 8. The elution fraction containing CAV Vp1 were analyzed by negative staining electron microscopy. As shown in FIG. 4B, CAV Vp1 virus-like particles were observed in this in vitro setting.

### Example 10. Expression of Ring2 ORF1 and ORF2 in Insect Cells

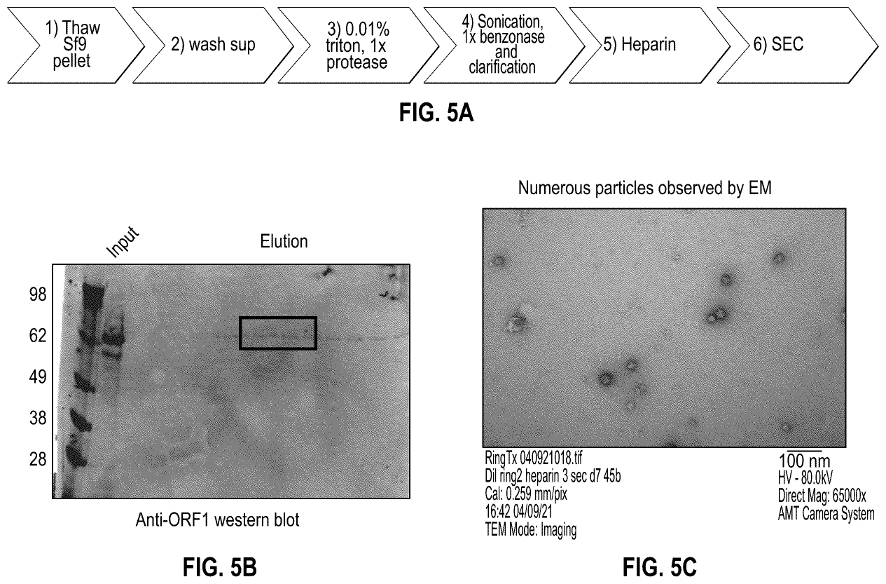

In this example, untagged Ring 2 ORF1 and ORF2 (a putative zinc-finger Ring 2 protein of unknown function) were cloned into a dual-expression baculovirus and co-expressed in Sf9 insect cells using a baculovirus system as described herein. Frozen pellet from a 1 L preparation of the Sf9 cells were resuspended in 60 mL cytosolic buffer (50 mM Tris pH 8, 50 mM NaCl, 1× protease inhibiter), vortexed, pipetted up-down and distributed into two 30 mL aliquotes in 50 mL conical tubes. The solutions were spun down for 20 min at 14,000 rpm using a fixed rotor centrifuge. The supernatant was collected into separate 50 mL conical tubes (“wash sup”). Each pellet was resuspended with 30 mL lysis buffer (50 mM Tris pH 8, 50 mM NaCl buffer, 1× protease inhibitor, 0.01-0.1% Triton), vortexed, and sonicated with four cycles. 1× benzonase was added and the solutions were spun for 20 min at 14,000 rpm using a fixed rotor centrifuge. The lysate was incubated for about 30 minutes to allow the benzonase to react to remove host cell DNA. The ORF1 was purified from the lysates using an affinity purification step (heparin column pH=8 with a high salt gradient elution) and VLPs, partially formed VLPs, and ORF1 proteins (putative capsomers) were separated using size-exclusion purification (SEC; GE Healthcare Sephacryl S-500 column). The resulting material was analyzed by electron microscopy (FIG. 5A-5C). Approximately 10¹¹ total VLP particles at approximately 109-10¹⁰ particles/ml concentration were purified, as determined by western blot analysis and electron microscopy. Such a high-titer VLP preparation can be used to define in vitro disassembly and re-assembly/encapsidation conditions for anellovector generation as described below.

### Example 11. Disassembly of Ring2 VLPs Using Urea

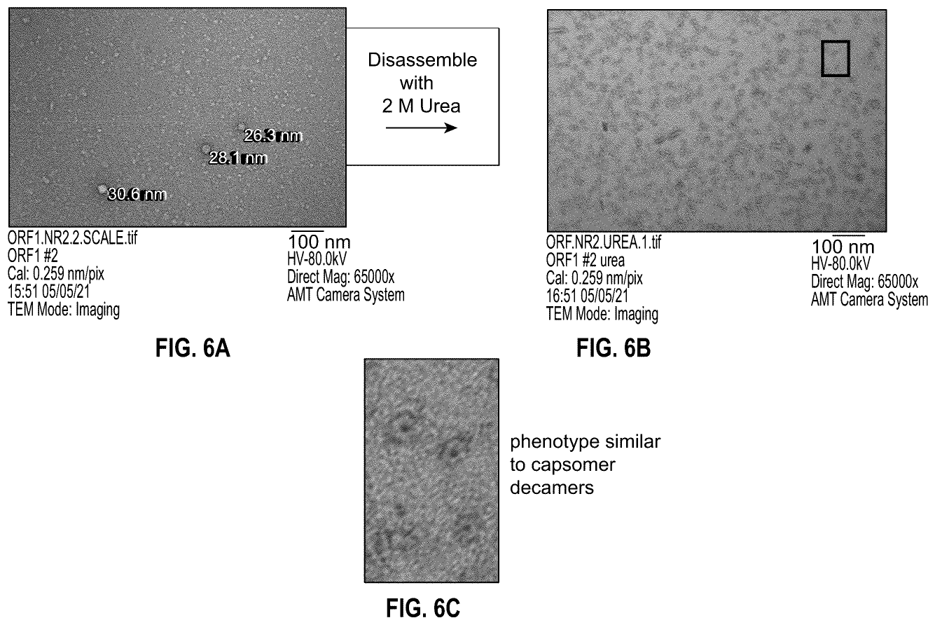

In this example, purified Ring2 VLPs were dissociated using a chaotropic agent. Briefly, purified Ring 2 VLPs were treated with urea at different concentrations, between 1 to 6 molar (M), to identify conditions sufficient to disassemble the particles. In one example, Ring 2 VLPs were treated with urea with a final concentration of 2M for approximately 10 minutes. The sample was observed by electron microscopy before (FIG. 6A) and after (FIG. 6B) urea treatment. Prior to treatment with urea, VLPs were observed with an estimated particle titer of 1×10⁹ to 1×10¹⁰ particles/ml. After treatment with urea, the VLPs were no longer observed. A new species, which appear to be hollow circles, like capsomers, was also observed (FIG. 6C).

In a further example, Ring 2 VLPs are dissociated with 2M urea, and the urea is dialyzed away in the absence or presence of mRNA encoding the mCherry reporter gene. In this example, mRNA is introduced at a molar concentration estimated to be approximately 5-times greater than the estimated number of VLPs prior to dissociation. The dissociated ORF1 and mRNA solution are incubated for approximately 30 minutes to allow complex formation before reassembly by dialysis. After the incubation period, disassembled ORF1 (i.e. VLPs treated with 2M urea) with or without mRNA are then dialyzed against 50 mM Tris pH 8.0 with 150 mM NaCl and 0.01% poloxamer to permit VLP reassembly. The initial VLPs, dissociated ORF1, reassembled ORF1 in the absence of nucleic acids, and reassembled ORF1 in the presence of nucleic acids are then screened by EM to confirm that the dissociation/re-assembly processes are successful as well as to estimate the amount of VLP recovered. It is contemplated that no VLPs will be observed for disassembled VLP or disassembled VLP dialyzed in the absence of mRNA. However, it is contemplated that VLPs prior to disassembly may be present at a particle titer of 1×10⁹ to 1×10¹⁰ particles/ml and the VLP formation observed after dialysis in the presence of mRNA may have a particle titer of 1×10⁷ to 1×10⁸ particles/ml. It is contemplated that VLP reassembly occurs when disassembled ORF1 is dialyzed/reassembled in the presence of mRNA, resulting in mRNA-encapsidated anellovectors.

### Example 12. Dissociation of Anellovirus VLPs and Reassembly Around Nucleic Acid Cargoes

In this example, Anellovirus ORF1 proteins produced and purified as described herein (e.g., wildtype ORF1 protein, chimeric ORF1 protein, or fragments thereof) will be disassembled and then reassembled in vitro. VLPs will be incubated under conditions sufficient to dissociate VLPs or viral capsids (e.g., as described herein), and then under conditions suitable enable reassembly, for example, around nucleic acid cargo (FIGS. 5A-5C). Exemplary nucleic acid cargo can include, without limitation, as double stranded DNA, single stranded DNA (ssDNA), or RNA that encodes a gene of interest to be delivered as a therapeutic agent. Exemplary conditions sufficient to dissociate VLPs or viral capsids include, but are not limited to, buffers of different pH, conditions of defined conductivity (salt content), conditions containing detergents (such as SDS, Tween, Triton), conditions containing chaotropic agents (such as Urea, e.g., as described herein) or conditions involving defined temperature and time (reannealing temperatures). Generally, nucleic acid cargo of defined concentration will be combined with Anellovirus ORF1 proteins of defined concentration and treated with conditions sufficient to permit nucleic acid encapsidation. The resulting particles can be subsequently purified using viral purification procedures in the art and/or as described herein.

Encapsidation of ssDNA Cargo

In this example, purified Anellovirus ORF1 proteins are treated with high salt in 2 M Urea to disassemble VLPs into dispersed protein or capsomers. The Anellovirus ORF1 proteins are then mixed with ssDNA and dialyzed against Tris pH 8.0 with 150 mM NaCl to permit VLP formation and ssDNA encapsidation. The subsequent complex is purified by SEC using to isolate anellovectors encapsidating ssDNA from non-encapsidated DNA. Anellovector assembly can be further evaluated by biophysical assessment such as DLS or electron microscopy, e.g., as described herein.

Encapsidation of mRNA Cargo with Wild-Type Anellovirus ORFJ

In this example, purified Anellovirus ORF1 proteins are treated with 1 M NaCl with 0.1% SDS dissociate oligomers or VLPs into dispersed protein or capsomers. The Anellovirus ORF1 proteins are then mixed with mRNA, such as an mRNA that encodes a gene of interest (e.g., GFP, mCherry, or EPO), and dialyzed against Tris pH 8.0 with 150 mM NaCl to permit VLP formation. The subsequent complex is purified by SEC using Tris pH 8.0 buffer to isolate anellovectors encapsidating mRNA. Anellovector assembly can be further evaluated by in vitro or in vivo readout, for example, by transducing cells and observing the expression of a reporter gene (in the case of mCherry or GFP) or through expression of a gene of interest (such as using an ELISA to detect the expression of a gene such as EPO).

Encapsidation of mRNA Cargo with Modified ORF1 Having mRNA Binding Region

In this example, packaging of mRNA cargo by Anellovirus ORF1 proteins is improved by modifying the Anellovirus ORF1 protein to harbor contact residues known to bind mRNA. For example, the ssDNA contact residues and/or the structural jellyroll beta strands known to contact ssDNA and/or the N-terminal arginine ARM can be replaced with components of an mRNA binding viral protein (e.g., MS2 coat protein) or other mRNA-binding protein to permit efficient binding and packaging of mRNA. This mRNA-binding chimeric ORF1 could be then treated with 1 M NaCl with 0.1% SDS dissociate oligomers or VLPs into dispersed protein or capsomers. The chimeric ORF1 would then be mixed with mRNA, such as an mRNA that translate a gene of interest such as GFP, mCherry or EPO, and dialyzed against Tris pH 8.0 with 150 mM NaCl to permit VLP formation. The subsequent complex is purified by SEC using Tris pH 8.0 buffer to isolate anellovectors encapsidating mRNA. Anellovector assembly can be further evaluated by in vitro or in vivo readout, for example, by transducing cells and observing the expression of the reporter gene (in the case of mCherry or GFP) or through expression of a gene of interest (such as using an ELISA to detect the expression of a gene such as EPO).

Encapsidation of ssDNA-mRNA Hybrid Cargo with ORF1

In this example, packaging of mRNA cargo by Anellovirus ORF1 proteins is improved by binding the mRNA molecule to ssDNA or modifying the mRNA transgene in such a way that that a section of the backbone would permit binding to the ssDNA contact residues of wildtype Anellovirus ORF1. For example, modified ssDNA that can bind Anellovirus ORF1 by virtue of its sugar-chain backbone but pair with mRNA non-covalently is mixed with the mRNA to produce a synthetic mRNA complex. Alternatively, a synthetic mRNA transgene can be synthesized with a section or sections of the mRNA molecule harboring a DNA backbone permitting binding and encapsidation with Anellovirus ORF1, while retaining the portion of the mRNA that encodes the gene to be delivered. Anellovirus ORF1 could be then treated with 1 M NaCl with 0.1% SDS dissociate oligomers or VLPs into dispersed protein or capsomers. The Anellovirus ORF1 would then be mixed with the synthetic mRNA (complexes or molecules), such as an mRNA that translate a gene of interest such as GFP, mCherry or EPO, and dialyzed against Tris pH 8.0 with 150 mM NaCl to permit VLP formation. The subsequent particle is purified by SEC using Tris pH 8.0 buffer to isolate anellovectors encapsidating mRNA. Anellovector assembly can be further evaluated by in vitro or in vivo readout by transducing cells and observing the expression of the reporter gene (in the case of mCherry or GFP) or through expression of a gene of interest (such as using an ELISA to detect the expression of a gene such as EPO).

### Example 13: Replication-Deficient Anellovectors and Helper Viruses

For replication and packaging of an anellovector, some elements (e.g., an ORF1, ORF1/1, ORF1/2, ORF2, ORF2/2, ORF2/3, and/or ORF2t/3 molecule, or a nucleic acid sequence encoding same) can be provided in trans. These include proteins or non-coding RNAs that direct or support DNA replication or packaging. Trans elements can, in some instances, be provided from a source alternative to the anellovector, such as a helper virus, plasmid, or from the cellular genome.

Other elements are typically provided in cis (e.g., a TATA box, cap site, initiator element, transcriptional start site, 5′ UTR conserved domain, three open-reading frame region, poly(A) signal, or GC-rich region). These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans.

Replication deficient viruses can be useful for controlling replication of an anellovector (e.g., a replication-deficient or packaging-deficient anellovector) in the same cell. In some instances, the virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient virus would drive the amplification and packaging of the anellovector. The packaged particles collected would thus be comprised solely of therapeutic anellovector, without contamination from the replication-deficient virus.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

### Example 14: Manufacturing Process for Replication-Competent Anellovectors

This example describes a method for recovery and scaling up of production of replication-competent anellovectors. Anellovectors are replication competent when they encode in their genome all the required nucleic acid elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic anellovector either in its linear or circular form is introduced into 5 E+05 adherent mammalian cells in a T75 flask by chemical transfection or into 5 E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1).

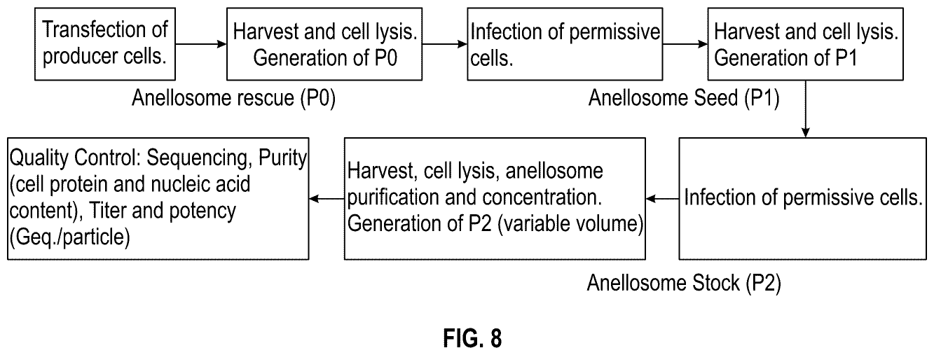

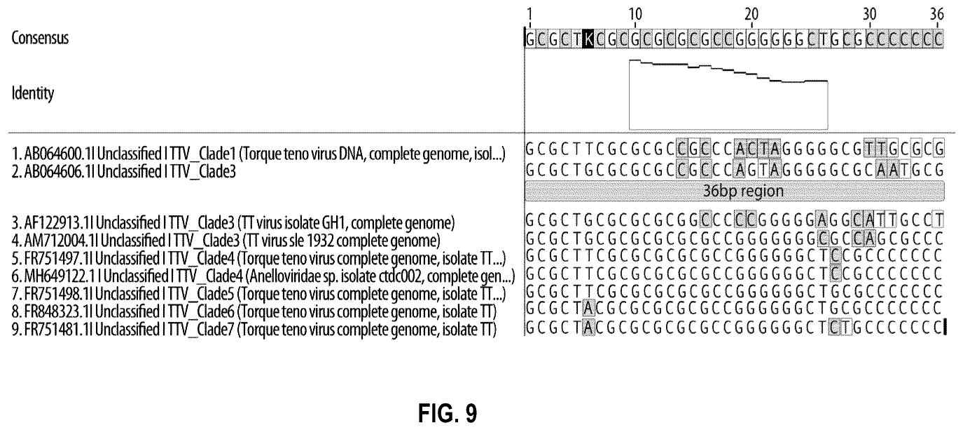

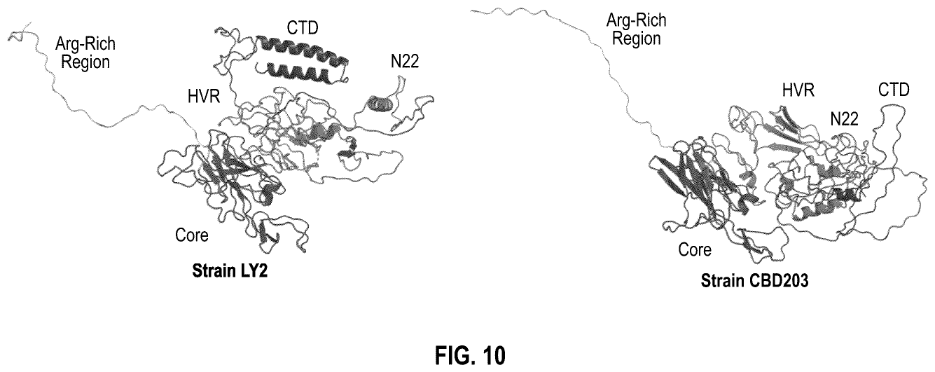

Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 8.

### Example 15: Manufacturing Process of Replication-Deficient Anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described herein.

### Example 16: Production of Anellovectors Using Suspension Cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described herein). The complementing plasmids can, in some instances, encode for viral proteins that have been deleted from the anellovector genome (e.g., an anellovector genome based on a viral genoe, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence.

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

### Example 17: Antibody Generation and Western Blot Analysis

This example describes generation of Ring 10 (also referred to herein as Ly1) antibodies and western blot analysis for determining expression, e.g., of Ring 10 ORF1 proteins. Ring 10 antibodies were generated by immunizing rabbits with a synthetic peptide representing one of three portions of the ORF1 protein (jelly roll residues 46-58: KIKRLNIVEWQPK (SEQ ID NO: 2687), spike domain residues 485-502: SPSDTHEPDEEDQNRWYP (SEQ ID NO: 2688), C-terminal domain residues 635-672: SEEEEESNLFERLLRQRTKQLQLKRRIIQTLKDLQKLE (SEQ ID NO: 2689)) conjugated to a carrier protein by an engineered N-terminal cysteine. Rabbits were immunized twice, and polyclonal antibodies were purified from bleeds using protein A purification (Custom Antibody Production, Life Technologies corporation). Western blot analysis was performed using NUPAGE 4-12% gels (ThermoFisher) transferred to nitrocellulose membranes using the Transblot Turbo system (BioRad). Membranes were blocked with blocking buffer (Licor), probed ˜16 hours with primary antibody, and detected using anti-rabbit IRDye infrared secondary antibodies and imaging system (Licor).

### Example 18: Construct Design, Cell Culture and Protein Expression/Purification

This example describes design of Ring 10 constructs, cell culture, and expression/purification of Ring 10 proteins. The Ring 10 ORF2 and ORF1 sequences were codon optimized for insect cells and with different ORF1 construct length variations (full-length ORF1, delARM with a deletion of residue 2-45, and delARM/delCTD with deletions of residues 2-45 and 552-672). The ORF2 and ORF1 constructs were cloned into the pFastBac Dual plasmid which was used to generate baculoviruses. To express the ORF1 proteins, Sf9 cells (Gibco™ 11496015) were infected by baculovirus with multiplicity of infection=1 and the cells were cultured for three days at 27° C. and harvested by centrifugation. The cells were lysed by treatment with 0.01% Triton X-100 (Sigma-Aldrich 11332481001), subjected to micro-fluidization, and treated with protease inhibitors (Thermo Scientific Halt Protease Inhibitor Cocktail, PI78438) and DNase (Benzonase®; Sigma). Cell lysate was subsequently purified using HiTrap Heparin affinity chromatography (Cytiva) followed by size-exclusion chromatography (HiPrep 16/60 Sephacryl S-500 HR; Cytiva).

### Example 19: Negative-Stained EM Data Collection and Analysis

This example describes visualization of anellovirus particles using negative stain electron microscopy. Jeol 1200EX transmission electron microscope was used for screening different Ring 10 constructs. 10 μl of sample was blotted on 400 mesh carbon support film (cf400-cu, EMS) for 30 seconds. After washing by ddH₂O for 30 seconds, the grid was stained by 0.75% of uranyl formate (UF) for 10 seconds before loading on the scope. Ring 10 delARM was further imaged at NanoImaging Service. 3 μl of 0.12 mg/ml was blotted to a continuous carbon grid and stained by 1% UF. Negative-stained electron microscopy was performed using a Thermo Fisher Scientific (Hillsboro, Oregon) Glacios Cryo Transmission Electron Microscope (cryo-TEM) operated at 200 kV and equipped with a TFS CETA-D 4×4 CMOS camera and a Falcon 4 direct electron detector.

### Example 20: Cryo-EM Data Collection and Data Analysis and Molecular Refinement

This example describes performing cryogenic electron microscopy (Cryo-EM) and data analysis, e.g., for determination of anellovirus particle structure. For the grid preparation, 3 μl of 0.3 mg/ml VLP sample was applied to a 1.2×1.3 graphene oxide grid. A total of 11,083 micrographs were collected from Glacios cryo-TEM (Thermo Fisher Scientific) operated at 200 kV with a Falcon 4 direct electron detector, at a nominal defocus range of −1.0-−2.5 m and accumulated dose of 19.59 e⁻/Å for a total of 15 frames in 3 minutes. The pixel size was 0.923 Å, with the magnification 150000×. Automated data-collection was carried out by Leginon software.

All micrographs were motion-corrected by Relion-4.0 implemented MotionCor2, and the contrast transfer function (CTF) parameters were estimated by Gctf. With manually picked particles from 20 micrographs to train the network, SPHIRE-crYOLO automatically picked 58,391 particles along with the PhosaurusNet network. All particles were extracted by Relion-4.0 and rescaled to 2-fold (pixel size 1.846 Å), followed by subsequence 2D classifications with 350 Å mask diameter to remove any junk particles. Two iterations of 2D classifications resulted in 11,185 particles, which were merged and reextracted to generate a de novo 3D initial model by Relion-4.0 with I1 symmetry. Notably, several similar initial models without symmetry imposed were obtained by Relion-4.0 and cisTEM. To obtain a better classification result, all particles were first subjected to a Refine3D with initial angular sampling 3.7° and local angular search 0.9° per step. The alignment parameters of each particle were transferred to a 3D classification with angular sampling interval 0.9° and local angular search 5° per step. 3D classification of the entire particle set attributed most of the particles into a single class. After CtfRefine and Bayesian polishing, the post-processing results in 3.98 Å resolution under the gold standard (with FSC=0.143).

The initial anellovirus TTMV-LY2 capsid monomer structure was predicted by TrRosetta, and Ring 10 structure was further predicted by RosettaCM. Structural refinement was performed by Rosetta and Phenix, and fine-adjusted by COOT. JA 20 and SAFIA structures are predicted by Alphafold.

### Example 21: Circular Dichroism Spectroscopy

This example describes performing circular dichroism spectroscopy to determine secondary structure of, e.g., an anellovirus ORF1 protein. To determine the secondary structure of the Ring 10 C-terminal domain, the peptides used to generate C-terminal antibodies (residues 635-672) were analyzed at 25.8 μM in PBS on a Jasco J-815 circular dichroism spectropolarimeter using 2 mm path length cell at ambient temperature. Each data set is the average of three consecutive scans. The secondary structure was determined by using CDPro software package, compared with a reference set containing 56 proteins (IBasis=10). The final secondary structure fractions were averaged over the results from three programs (SELCON3, CDSSTR, CONTINLL) in CDPro.

### Example 22: Anellovirus Particle Structure

This example describes determination of the particle structure of an exemplary Anellovirus.

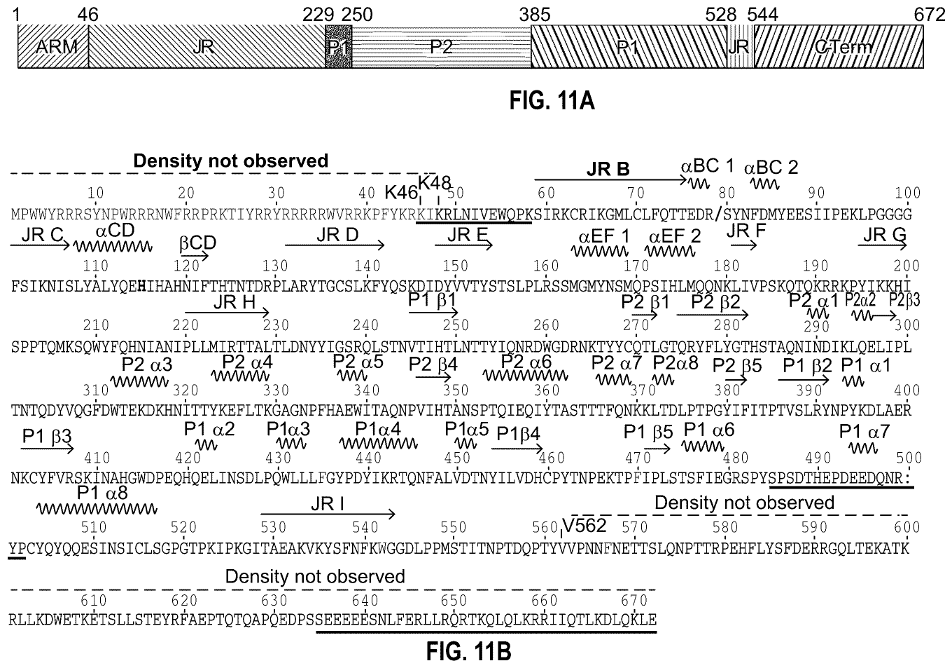

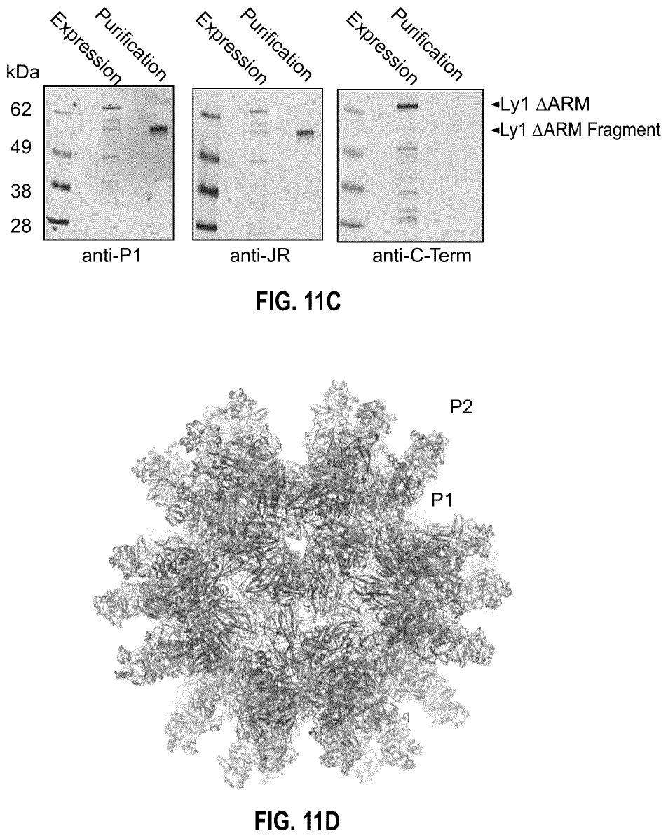

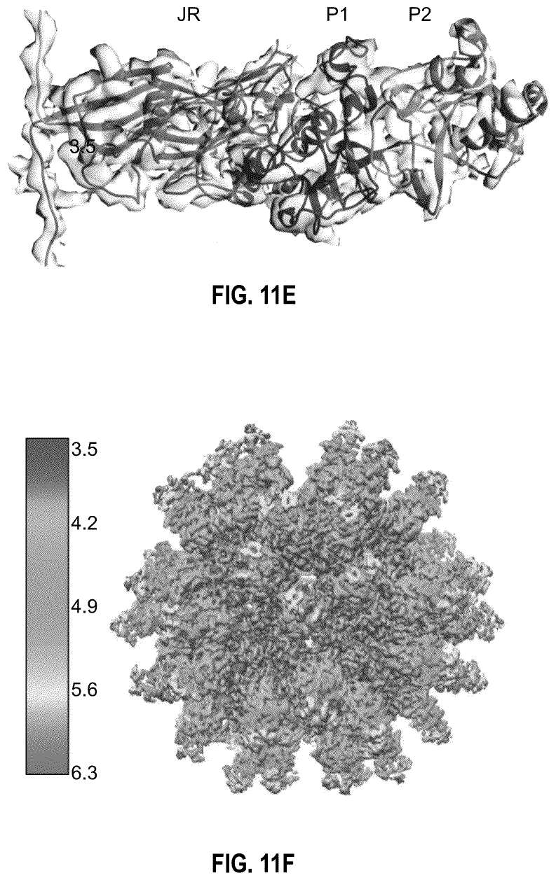

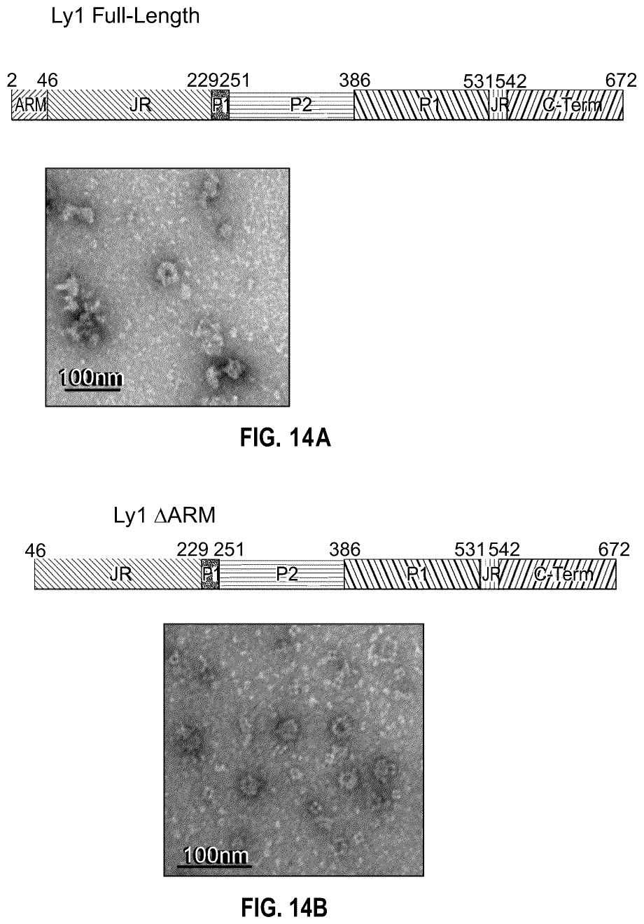

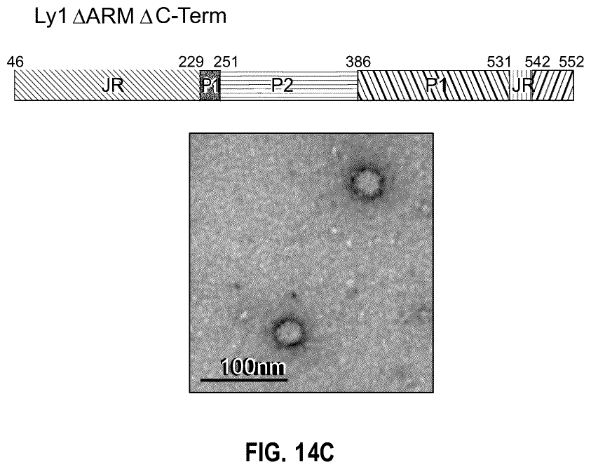

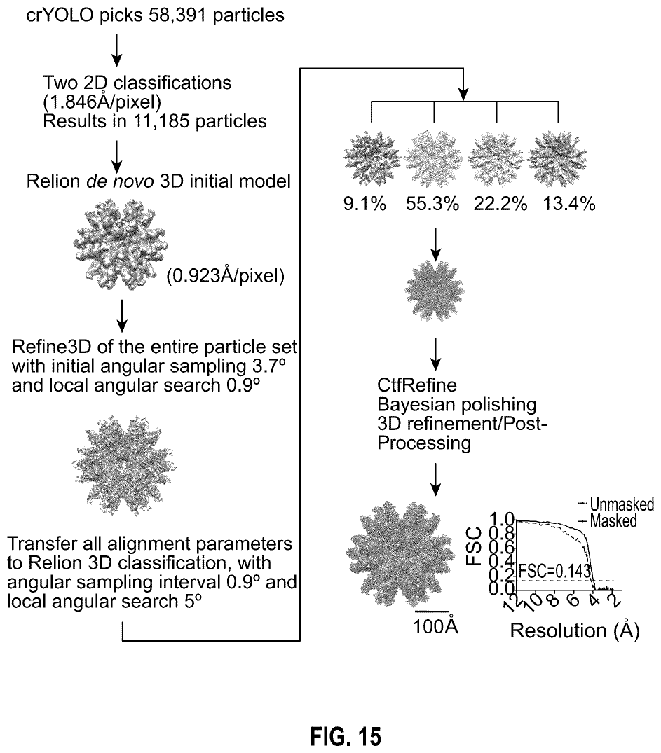

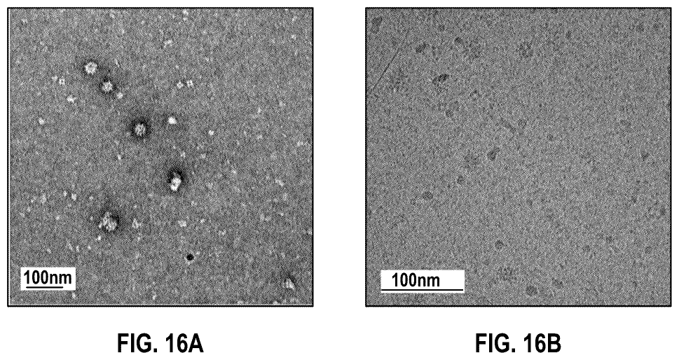

Initial efforts to study the structure of a virus-like particle (VLP) derived from a TTMV isolate described herein as Ring 10, were done with the full-length ORF1 (residues 1-672) expressed in insect cells (Sf9). Full-length ORF1 was visualized by electron microscopy (as described in Example 19) and was observed to assemble into particles ˜32 nm in diameter (FIG. 14A), similar to previously reported estimates of anellovirus size. However, full-length ORF1 particles lacked the homogeneous symmetry expected of viral particles (FIG. 14A). Genome packaging by ARM-containing viruses is believed to occur when the positive arginine residues bind a negatively charged genome, overcoming electrostatic repulsion of the ARM to permit ORF1 assembly. Therefore, Ring 10 delARM, an ORF1 construct wherein residues 2-45 are deleted, was designed as described in Example 18 (FIGS. 11A, 11B, and 14B) to facilitate more efficient ORF1 assembly in the absence of viral DNA. Ring 10 delARM expression was confirmed using a rabbit polyclonal antibody raised against a spike domain peptide (residues 485-502; FIG. 11B) as described in Example 17. A band consistent with Ring 10 delARM was observed by western blot analysis (expected mass of 73.3 kDa; FIG. 11C) performed as described in Example 17. Ring 10 delARM yielded an ORF1 band above the 62-kDa marker (FIG. 11C). The difference in apparent molecular weight of Ring 10 delARM ORF1 after initial expression and after purification suggested proteolysis. To identify the site of proteolysis, polyclonal antibodies to peptides from the extreme N- and C-termini of Ring 10 delARM (residues 46-58 and residues 635-672, respectively; FIG. 11B) were generated as described in Example 17, and the presence of the N-terminal peptide of Ring 10 delARM on the purified fragment and the absence of the C-terminal peptide were confirmed (FIG. 11C).

Electron microscopy (EM) analysis of the Ring 10 delARM fragment showed the VLPs formed were more homogeneous and symmetric in morphology relative to full-length ORF1 (FIGS. 14A and 14B). The formation of homogeneous VLPs following genetic removal of the N-terminus and proteolysis of the C-terminus has been observed in another JR-containing virus, hepatitis E (HEV). To determine if the C-terminus of Ring 10 ORF1 is required for particle formation, VLPs from construct Ring 10 ORF1 delARM delCTD (wherein residues 2-45 and 552-672 are deleted) were generated as described in Example 18. Ring 10 ORF1 delARM delCTD produced VLPs of similar symmetry to Ring 10 ORF1 delARM (FIGS. 14B and 14C). These results suggest that proteolysis of the ORF1 C-terminus may be a natural part of anellovirus formation. In light of recent evidence that the C-terminal region of ORF1 is the immunodominant region of anelloviruses, its excision from the mature particle would be consistent with the immune evasion properties of anelloviruses.

The structure of the Ring 10 delARM particle was determined using cryo-EM to 3.98 Å resolution as described in Example 20 (FIG. 11D-11F, 15, 16A, and 16B; Table S1). The anellovirus particle was shown to be formed by sixty ORF1 fragments organized in an icosahedral T=1 symmetry (FIG. 11D). Electron density for residues 48-562 was observed (FIG. 11B). The resulting mass of the observed Ring 10 fragment was calculated to be ˜59.8 kDa. The N-terminal region (residues 46-228) forms part of the canonical 8-β-strand JR domain (β strands named B to H by convention). Unexpectedly, the eighth and final β-strand in the JR (strand I, residues 531-542) is located just prior to the C-terminal domain (FIG. 11B). The resulting fold of the ORF1 protomer has residues at the N- and C-termini generating the JR domain at the particle core while the intrastrand residues form the exterior of the particle surface. Intrastrand insertions forming the viral particle exterior can be found in other JR-containing viruses such as adeno-associated virus (AAV) and canine parvovirus (CPV). However, while the intrastrand insertions for AAV2 (228 residues) and CPV (227 residues) are between G and H β-strands, the 298-residue intrastrand insertion in Ring 10 is significantly larger and lies between β-strands H and I. The intrastrand region (residues 229-530) extends from the JR domain to form a structure herein referred to as a spike domain. The spike domain is formed by two globular domains: the spike P1 domain (residues 229-250 and 386-530) and the spike P2 domain (residues 251-385; FIG. 11B, 11E).

TABLE S1
Cryo-EM data collection, refinement and validation
statistics of TTMV-Ring 10 delARM.

| | Ring 10 delARM |
|---|---|
| Data collection and processing | |
| Magnification | 150000x |
| Voltage (kV) | 200 |
| Electron exposure (e−/Å²) | 1.31 |
| Defocus range (μm) | −0.5~−2.5 |
| Pixel size (Å) | 0.923 |
| Symmetry imposed | I1 |
| Initial particle images (no.) | 58391 |
| Final particle images (no.) | 6271 |
| Map resolution (Å) | 3.98/0.143 |
| FSC threshold | |
| Map resolution range (Å) | 3.9-6 |
| Refinement | |
| Initial model used (PDB code) | |
| Model resolution (Å) | 3.98/0.143 |
| FSC threshold | |
| Model resolution range (Å) | 3.9-6 |
| Map sharpening B factor (Å²) | −152.851 |
| Model composition | 253440/30900/0 |
| Non-hydrogen atoms | |
| Protein residues | |
| Ligands | |
| B factors (Å²) | 30.00/243.72/133.17 |
| Protein | |
| Ligand | |
| R.m.s. deviations | 0.007/1.282 |
| Bond lengths (Å) | |
| Bond angles (°) | |
| Validation | 2.36/25.43/0 |
| MolProbity score | |
| Clashscore | |
| Poor rotamers (%) | |
| Ramachandran plot | 92.6/6.62/0.78 |
| Favored (%) | |
| Allowed (%) | |
| Disallowed (%) | |
| CC (mask) | 0.81 |
| MolProbility | 2.36 |
| Clash Score | 25.43 |

### Example 23: Anellovirus Structural Jelly Roll Domains

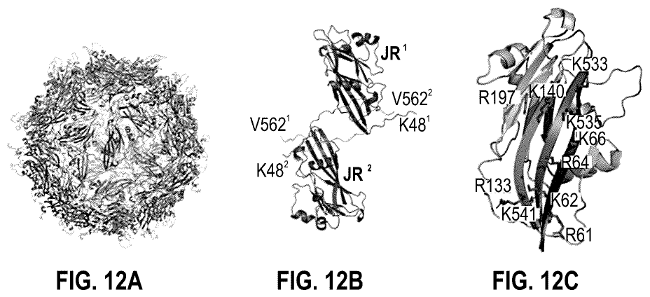

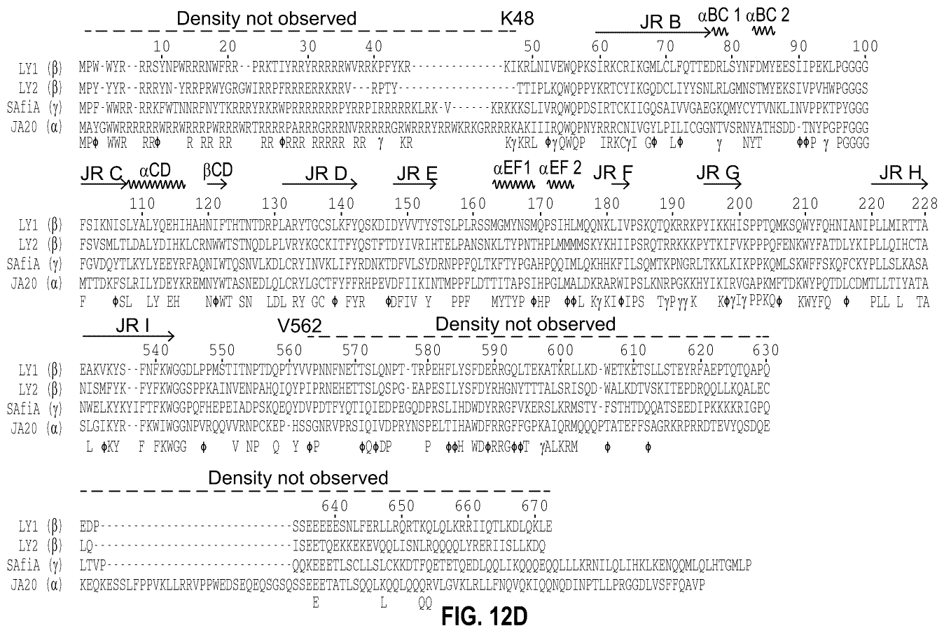

This example describes the structure of anellovirus structural jelly roll (JR) domains. Sixty Ring 10 JR domains form the core of the virus particle (FIG. 12A-12D). The β-strands form β-sheets which are characterized by a C-H-E-F pattern on the core's exterior and B-I-D-G pattern on the core's interior. The N-terminus of strand B is oriented to place the ARM on the interior of the core, where it is positioned to bind the viral genome. The observed C-terminal residues (545-562) extend from the C-terminus of P3-strand I on the interior of the particle and thread through JR domains on the 2-fold axis to contact the neighboring JR domain (FIG. 12B).

In several JR-containing viruses, positively charged residues (arginine and lysine) oriented internally on strands B, I, D, and G are expected to bind the negatively charged viral genome (FIG. 12C). In Ring 10, basic residues Arg61, Lys62, Arg64, Lys66 (β-strand B), Lys140 (β-strand D), Arg197 (β-strand G), Lys533, Lys535, and Lys541 (β-strand I) are all oriented toward the particle interior and are likely responsible, together with the ARM motif, for binding the negatively charged viral genome. Notably, density suggesting bound nucleic acid was not observed, possibly because the ARM deletion prevented nucleic acid binding or because any host cell nucleic acid encapsidated by the VLP would be too heterogeneous for detection. Alignment of anellovirus ORF1 sequences revealed that several of these putative DNA-binding residues are conserved across species, which supports their role in DNA-binding (FIG. 12D).

### Example 24: Anellovirus Spike Domains

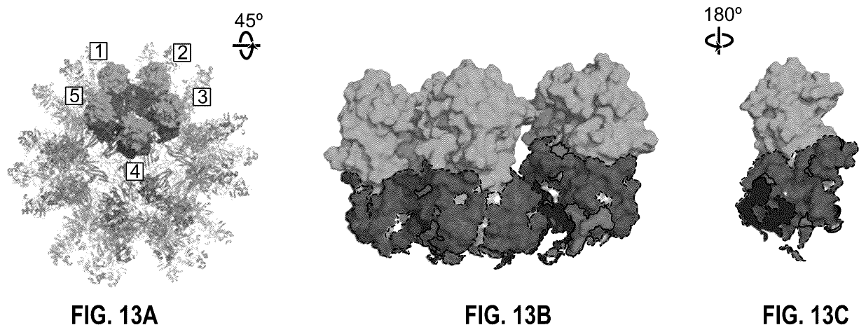

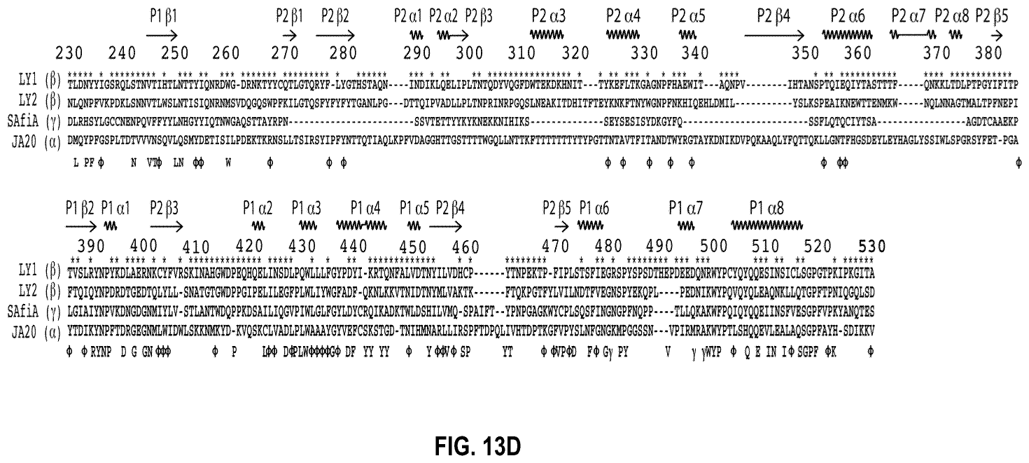

This example describes the structure of exemplary anellovirus spike domains, which include the P1 and P2 domains (e.g., as described herein). Residues 229 to 530 form the spike domain that extends ˜6 nm from the JR core (FIG. 13A-13D). A β strand (residues 245-250) extending from JR 3-strand H is the first component of the spike P1 domain and is N-terminal to the spike P2 domain (residues 251-385). The previously described hypervariable region (HVR) of ORF1 comprises the majority of the spike P2 domain. The remaining residues of the spike domain (residues 386-530) form five additional 1-strands and eight helices, which, together with the residues 245-250 strand, fold into the spike P1 domain. The local resolution of P1 is only slightly lower than the JR domain (˜4-4.5 Å), while the resolution of P2 is within 5-6 Å. This is likely a consequence of both being further from the radius of gyration and some flexibility of the HVR residues.

Neighboring spike domains pack together around the five-fold symmetry axis to form a ringed structure of 5 spike domains henceforth called the crown (FIG. 13A, 13B). A receptor for anelloviruses has not been identified to date. Given the diverse tropism of different anellovirus strains, it is possible that residues of the spike P2 HVR, which are the most surface-exposed, are involved in viral attachment and infection. However, the hypervariable sequence of the spike P2 domain may serve to aid in immune evasion rather than harboring a receptor-binding motif. If this is the case, a receptor-binding motif on the better conserved spike P1 domain, or even on the surface of the JR core, may exist.

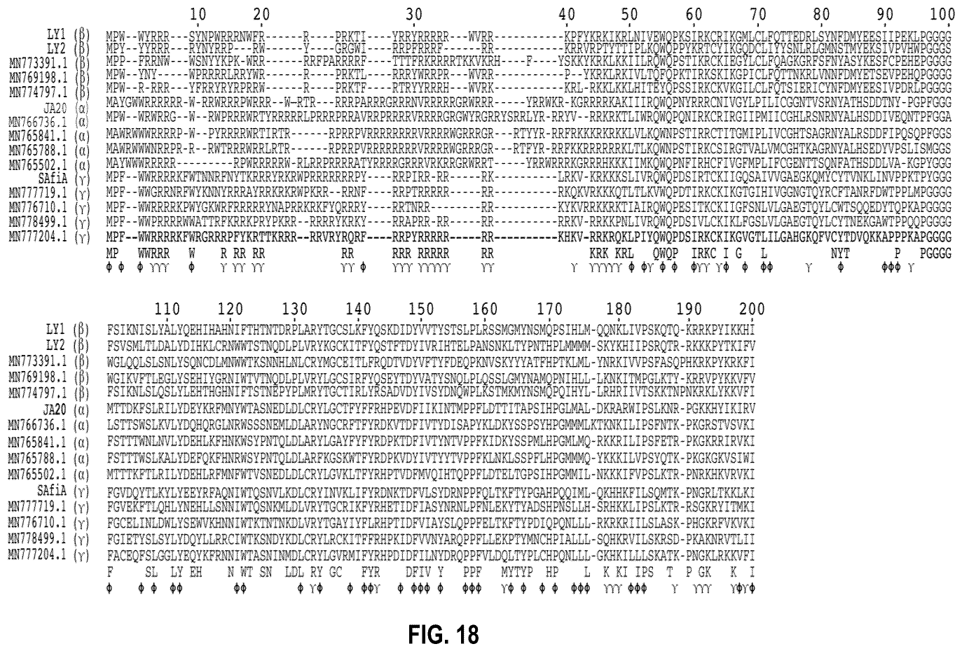

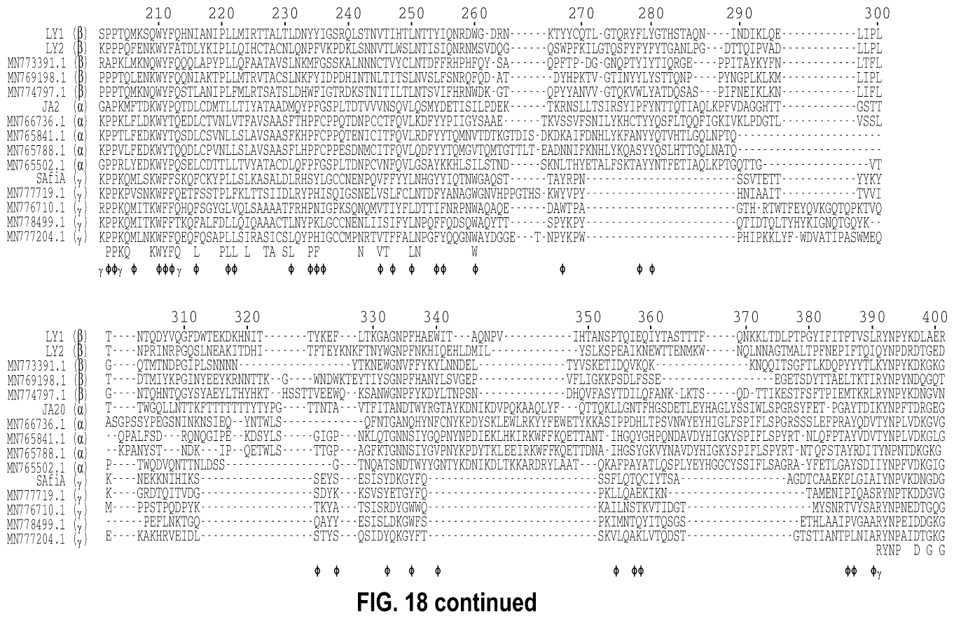

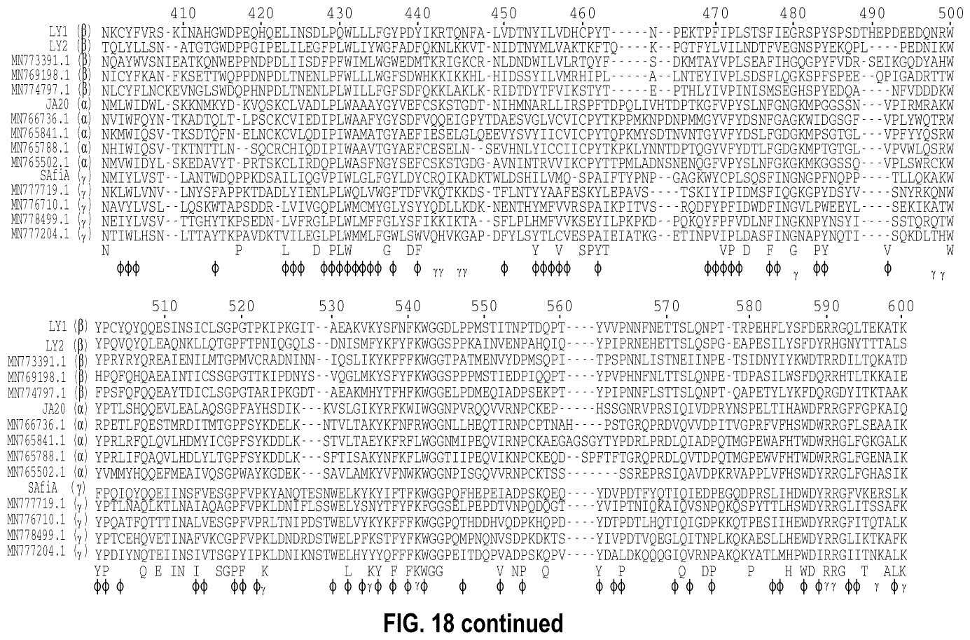

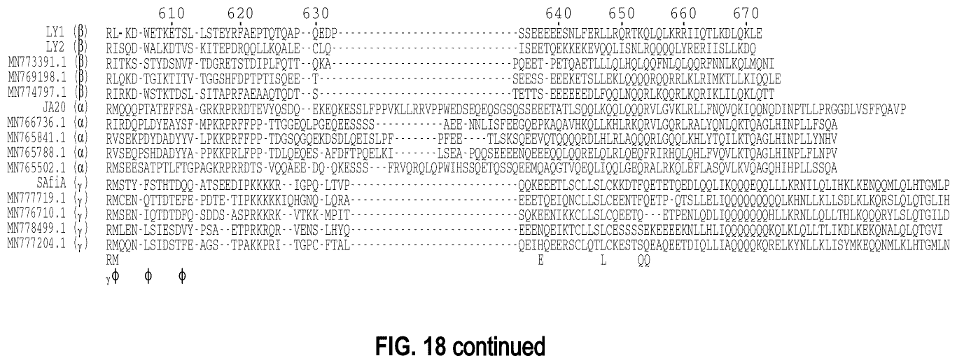

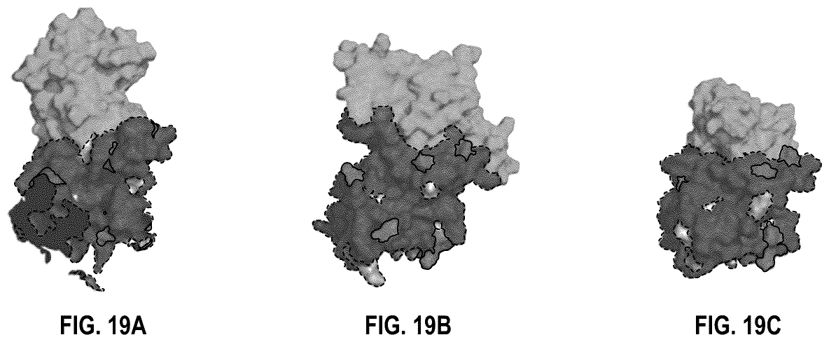

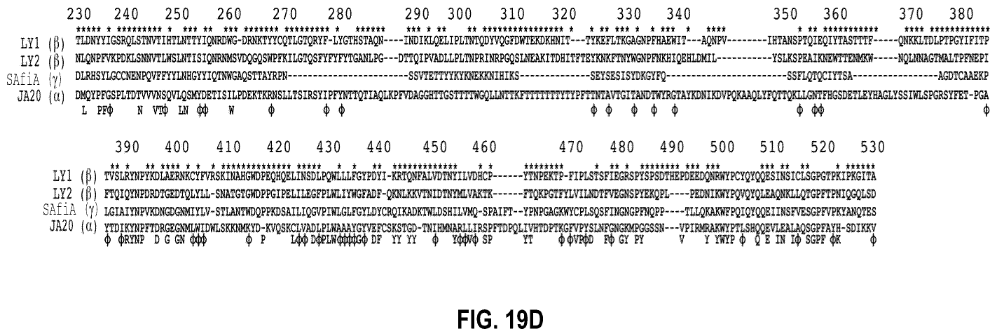

Sequence alignment of the Ring 10 spike domain with other anelloviruses did not readily identify conserved spike surface residues (FIG. 13B-13D, FIG. 18; sequences reproduced below). In fact, the Ring 10 residues that are more conserved between other anelloviruses (e.g., Asp396, Pro429, Trp431, Gly437, Pro461, Phe477, Pro483, Trp500, Tyr501, Pro502, Gly518 and Pro519) are internal spike P1 residues supporting its globular fold. A few basic residues are semi-conserved on the spike P1 surface (e.g., Arg390, Arg481, Arg499, and Arg523) which could contribute to the particle's ability to bind heparin resin. A few semi-conserved hydrophobic or aromatic residues on spike P1 (e.g., Leu231, I1e236, Val245, Tyr404, Gly414, I1e424, Leu428, Leu432, Val450, I1e478, and Tyr484) are at least partially surface-exposed and could represent a conserved receptor binding surface (FIG. 13B-13D). If this is the case, then it is attractive to posit that anelloviruses have evolved their novel elongated H—I intrastrand spike domain to sterically hinder antibody binding to their cellular receptor-binding site present on the P1 surface using domain P2 which is able to tolerate highly diverse amino acid substitutions. This would allow diverse anelloviruses to repeatedly infect human hosts with minimal recognition and neutralization by the immune system.

Sequences included in sequence alignments (FIG. 18)
>Ly1 (Ring10)_orf1
(SEQ ID NO: 1012)
MPWWYRRRSYNPWRRRNWERRPRKTIYRRYRRRRRWVRRKPFYKRKIKRLNIVEWQPKSIRKCRIKGMLCLFQT
TEDRLSYNFDMYEESIIPEKLPGGGGFSIKNISLYALYQEHIHAHNIFTHINTDRPLARYTGCSLKFYQSKDID
YVVTYSTSLPLRSSMGMYNSMQPSIHLMQQNKLIVPSKQTQKRRKPYIKKHISPPTQMKSQWYFQHNIANIPLL
MIRTTALTLDNYYIGSRQLSTNVTIHTLNTTYIQNRDWGDRNKTYYCQTLGTQRYFLYGTHSTAQNINDIKLQE
LIPLINTQDYVQGFDWTEKDKHNITTYKEFLTKGAGNPFHAEWITAQNPVIHTANSPTQIEQIYTASTTTFQNK
KLTDLPTPGYIFITPTVSLRYNPYKDLAERNKCYFVRSKINAHGWDPEQHQELINSDLPQWLLLFGYPDYIKRT
QNFALVDTNYILVDHCPYTNPEKIPFIPLSTSFIEGRSPYSPSDTHEPDEEDQNRWYPCYQYQQESINSICLSG
PGTPKIPKGITAEAKVKYSENFKWGGDLPPMSTITNPTDQPTYVVPNNFNETTSLQNPTTRPEHFLYSFDERRG
QLTEKATKRLLKDWETKETSLLSTEYRFAEPTQTQAPQEDPSSEEEEESNLFERLLRQRTKQLQLKRRIIQTLK
DLQKLE
>Ly2 (Ring2) orf1
(SEQ ID NO: 58)
MPYYYRRRRYNYRRPRWYGRGWIRRPFRRRFRRKRRVRPTYTTIPLKQWQPPYKRTCYIKGQDCLIYYSNLRLG
MNSTMYEKSIVPVHWPGGGSFSVSMLTLDALYDIHKLCRNWWTSTNQDLPLVRYKGCKITFYQSTFTDYIVRIH
TELPANSNKLTYPNTHPLMMMMSKYKHIIPSRQTRRKKKPYTKIFVKPPPQFENKWYFATDLYKIPLLQIHCTA
CNLQNPFVKPDKLSNNVTLWSLNTISIQNRNMSVDQGQSWPFKILGTQSFYFYFYTGANLPGDTTQIPVADLLP
LTNPRINRPGQSLNEAKITDHITFTEYKNKFTNYWGNPENKHIQEHLDMILYSLKSPEAIKNEWTTENMKWNQL
NNAGTMALTPFNEPIFTQIQYNPDRDTGEDTQLYLLSNATGTGWDPPGIPELILEGFPLWLIYWGFADFQKNLK
KVINIDTNYMLVAKTKFTQKPGTFYLVILNDTFVEGNSPYEKQPLPEDNIKWYPQVQYQLEAQNKLLQTGPFTP
NIQGQLSDNISMFYKFYFKWGGSPPKAINVENPAHQIQYPIPRNEHETTSLQSPGEAPESILYSFDYRHGNYTT
TALSRISQDWALKDTVSKITEPDRQQLLKQALECLQISEETQEKKEKEVQQLISNLRQQQQLYRERIISLLKDQ
>MN773391.1|Beta|245_2239_2_fow
(SEQ ID NO: 2746)
MPPFRRNWWSNYYKPKWRRRRFPARRRRFTTTFRKRRRRIKKVKRHFYSKKYKRKLKKIILRQWQPSTIKRCKI
EGYLCLFQAGKGRFSFNYASYKESFCPEHEPGGGGWGLQQLSLSNLYSQNCDLMNWWTKSNNHLNLCRYMGCEI
TLFRQDTVDYVFTYFDEQPKNVSKYYYATFHPTKLMLYNRKIVVPSFASQPHKRKPYKRKFIRAPKLMKNQWYF
QQQLAPYPLLQFAATAVSLNKMFGSSKALNNNCTVYCLNTDFFRHPHFQYSAQPFTPDGGNQPTYIYTIQRGEP
PITAYKYFNLIFLGQTMTNDPGIPLSNNNNYTKNEWGNVFFYKYLNNDELTYVSKETIDQVKQKKNQQITSGFT
LKDQPYYYTLKYNPYKDKGKGNQAYWVSNIEATKQNWEPPNDPDLIISDFPFWIMLWGWEDMTKRIGKCRNLDN
DWILVLRTQYFSDKMTAYVPLSEAFIHGQGPYFVDRSEIKGQDYAHWYPRYRYQREAIENILMTGPMVCRADNI
NNIQSLIKYKFFFKWGGTPATMENVYDPMSQPITPSPNNLISTNEIINPETSIDNYIYKWDTRRDILTQKATDR
ITKSSTYDSNVFTDGRETSTDIPLFQTTQKAPQEETPETQAETLLLQLHQLQQFNLQLQQRENNLKQLMQNI
>MN769198.1|Beta|265_2235_1_fow
(SEQ ID NO: 2747)
MPWYNYWPRRRRLRRYWRRPRKTLRRRYWRRPRWVRRPYKRKLRKIVLTQFQPKTIRKSKIKGPICLFQTINKR
LVNNFDMYETSEVPEHQPGGGGWGIKVFTLEGLYSEHIYGRNIWTVTNQDLPLVRYLGCSIRFYQSEYTDYVAT
YSNQLPLQSSLGMYNAMQPNIHLLLKNKITMPGLKTYKRRVPYKKVFVPPPTQLENKWYFQQNIAKTPLLMTRV
TACSLNKFYIDPDHINTNLTITSLNVSLFSNRQFQDATDYHPKTVGTINYYLYSTTQNPPYNGPLKLKMLIPLT
DTMIYKPGINYEEYKRNNTTKGWNDWKTEYTIYSGNPFHANYLSVGEPVFLIGKKPSDLESSEEGETSDYTTAE
LIKTIRYNPYNDQGQTNICYFKANFKSETTWQPPDNPDLINENLPFWLLLWGFSDWHKKIKKHLHIDSSYILVM
RHIPLALNTEYIVPLSDSFLQGKSPFSPEEQPIGADRTTWHPQFQHQAEAINTICSSGPGTTKIPDNYSVQGLM
KYSFYFKWGGSPPPMSTIEDPIQQPTYPVPHNFNLTTSLQNPETDPASILWSFDQRRHTLTKKAIERLQKDIGI
KTITVTGGSHFDPTPTISQEETSEESSEEEKETSLLEKLQQQQRQQRRLKLRIMKILLKIQQLE
>MN774797.1|Beta|244_2262_1_fow
(SEQ ID NO: 2748)
MPWRRRRYFRRYRYRPRRWRPRKTFRTRYYRRRHWVRKKRLRKKLKKLHITEYQPSSIRKCKVKGILCLFQTSI
ERICYNFDMYEESIVPDRLPGGGGFSIKNLSLQSLYLEHTHGHNIFTSTNEPYPLMRYTGCTIRLYRSADVDYI
VSYDNQWPLKSTMKMYNSMQPQIHYLLRHRIIVTSKKTNPNKRKLYKKVFIPPPTQMKNQWYFQSTLANIPLFM
LRTSATSLDHWFIGTRDKSTNITILTLNTSVIFHRNWDKGTQPYYANVVGTQKVWLYATDQSASPIFNEIKLKN
LIFLGNTQHNTQGYSYAEYLTHYHKTHSSTTVEEWQKSANWGNPFYKDYLTNPSNDHQVFASYTDILQFANKLK
TSQDTTIKESTFSFTPIEMTKRLRYNPYKDNGVNNLCYFLNCKEVNGLSWDQPHNPDLINENLPLWILLFGFSD
FQKKLAKLKRIDTDYTFVIKSTYTEPTHLYIVPINISMSEGHSPYEDQANFVDDDKWFPSFQFQQEAYTDICLS
GPGTARIPKGDTAEAKMHYTFHFKWGGELPDMEQIADPSEKPTYPIPNNFLSTTSLQNPTQAPETYLYKFDQRG
DYITKTAAKRIRKDWSTKTDSLSITAPRFAEAAQTQDTSTETTSEEEEEEDLFQQLNQQRLKQQRLKQRIKLIL
QKLQTT
>AF122914.3|Alpha|417_2726_3_fow (JA20)
(SEQ ID NO: 1020)
MAYGWWRRRRRRWRRWRRRPWRRRWRTRRRRPARRRGRRRNVRRRRRGRWRRRYRRWKRKGRRRRKAKIIIRQW
QPNYRRRCNIVGYLPILICGGNTVSRNYATHSDDTNYPGPFGGGMITDKFSLRILYDEYKREMNYWTASNEDLD
LCRYLGCTFYFFRHPEVDFIIKINTMPPFLDTTITAPSIHPGLMALDKRARWIPSLKNRPGKKHYIKIRVGAPK
MFTDKWYPQTDLCDMILLTIYATAADMQYPFGSPLIDTVVVNSQVLQSMYDETISILPDEKIKRNSLLTSIRSY
IPFYNTTQTIAQLKPFVDAGGHTTGSTTTTWGQLLNTTKFTTTTTTTYTYPGITNTAVIFITANDTWYRGTAYK
DNIKDVPQKAAQLYFQTTQKLLGNTFHGSDETLEYHAGLYSSIWLSPGRSYFETPGAYTDIKYNPFTDRGEGNM
LWIDWLSKKNMKYDKVQSKCLVADLPLWAAAYGYVEFCSKSTGDTNIHMNARLLIRSPFTDPQLIVHTDPTKGF
VPYSLNFGNGKMPGGSSNVPIRMRAKWYPTLSHQQEVLEALAQSGPFAYHSDIKKVSLGIKYRFKWIWGGNPVR
QQVVRNPCKEPHSSGNRVPRSIQIVDPRYNSPELTIHAWDFRRGFFGPKAIQRMQQQPTATEFFSAGRKRPRRD
TEVYQSDQEKEQKESSLFPPVKLLRRVPPWEDSEQEQSGSQSSEEETATLSQQLKQQLQQQRVLGVKLRLLENQ
VQKIQQNQDINPTLLPRGGDLVSFFQAVP
>MN766736.1|Alpha|422_2692_2_fow
(SEQ ID NO: 2749)
MPWWRWRRGWRWPRRRWRTYRRRRRLPRRRPRRAVRRPRRRRVRRRRGRGWYRGRRYSRRLYRRRYVRRKRKTL
IWRQWQPQNIRKCRIRGIIPMIICGHLRSNRNYALHSDDIVEQNTPFGGALSTTSWSLKVLYDQHQRGLNRWSS
SNEMLDLARYNGCRFTFYRDKVTDFIVTYDISAPYKLDKYSSPSYHPGMMMLKTKNKILIPSFNTKPKGRSTVS
VKIKPPKLFLDKWYTQEDLCTVNLVTFAVSAASFTHPFCPPQTDNPCCTFQVLKDFYYPIIGYSAAETKVSSVE
SNILYKHCTYYQSFLTQQFIGKIVKLPDGTLVSSLASGPSSYPEGSNINKNSIEQYNTWLSQFNTGANQHYNFC
NYKPDYSKLEWLRKYYFEWETYKKASIPPDHLTPSVNWYEYHIGLFSPIFLSPFRSSSLEFPRAYQDVTYNPLV
DKGVGNVIWFQYNTKADTQLTLPSCKCVIEDIPLWAAFYGYSDFVQQEIGPYTDAESVGLVCVICPYTKPPMKN
PDNPMMGYVFYDSNFGAGKWIDGSGFVPLYWQTRWRPETLFQESTMRDITMTGPFSYKDELKNTVLTAKYKFNF
RWGGNLLHEQTIRNPCPTNAHPSTGRQPRDVQVVDPITVGPRFVFHSWDWRRGELSEAAIKRIRDQPLDYEAYS
FMPKRPRFFPPTTGGEQLPGEQEESSSSAEENNLISFEEGQEPKAQAVHKQLLKHLRKQRVLGQRLRALYQNLQ
KTQAGLHINPLLFSQA
>MN765841.1|Alpha|389_2635_2_fow
(SEQ ID NO: 2750)
MAWRWWWRRRRPWPYRRRRWRTIRTRRPRRPVRRRRRRRRVRRRRWGRRRGRRTYYRRRFRKKKRKRKKLVLKQ
WNPSTIRRCTITGMIPLIVCGHTSAGRNYALRSDDFIPQSQPFGGSFSTTTWNLNVLYDEHLKFHNKWSYPNTQ
LDLARYLGAYFYFYRDPKTDFIVTYNTVPPFKIDKYSSPMLHPGMLMQRKKRILIPSFETRPKGKRRIRVKIKP
PTLFEDKWYTQSDLCSVNLLSLAVSAASFKHPFCPPQTENICITFQVLRDFYYTQMNVTDTKGTDISDKDKAIF
DNHLYKFANYYQTVHTLGQLNPTQQPALFSDRQNQGIPEKDSYLSGIGPNKLQTGNNSIYGQPNYNPDIEKLHK
IRKWFFKQETTANTIHGQYGHPQNDAVDYHIGKYSPIFLSPYRINLQFPTAYVDVTYNPLVDKGLGNKMWIQSV
TKSDTQFNELNCKCVLQDIPIWAMATGYAEFIESELGLQEEVYSVYIICVICPYTQPKMYSDTNVNTGYVFYDS
LFGDGKMPSGTGLVPFYYQSRWYPRLRFQLQVLHDMYICGPFSYKDDLKSTVLTAEYKFRFLWGGNMIPEQVIR
NPCKAEGAGSGYTYPDRLPRDLQIADPQTMGPEWAFHTWDWRHGLFGKGALKRVSEKPDYDADYYVLPKKPRFF
PPTDGSQGQEKDSDLQEISLPFPFEETLSKSQEEVQTQQQQRDLHLRLAQQQRLGQQLKHLYTQILKTQAGLHI
NPLLYNHV
>MN765502.1|Alpha|417_2624_3_fow
(SEQ ID NO: 2751)
MAYWWWRRRRRRPWRRRRRWRLRRPRRRRATYRRRRGRRRVRKRRGRWRRTYRRWRRRKGRRRHKKKIIMKQWQ
PNFIRHCFIVGFMPLIFCGENTTSQNFATHSDDLVAKGPYGGGMITTKFTLRILYDEHLRFMNFWTVSNEDLDL
CRYLGVKLTFYRHPTVDFMVQIHTQPPFLDTELTGPSIHPGMMILNKKKIFVPSLKTRPNRKHKVRVKIGPPRL
YEDKWYPQSELCDTTLLTVYATACDLQFPFGSPLTDNPCVNFQVLGSAYKKHLSILSTNDSKNLTHYETALFSK
TAYYNTFETIAQLKPTGQTTGVTPTWQDVQNTINLDSSGINQATSNDTWYYGNTYKDNIKDLTKKARDRYLAAT
QKAFPAYATLQSPLYEYHGGCYSSIFLSAGRAYFETLGAYSDIIYNPFVDKGIGNMVWIDYLSKEDAVYTPRIS
KCLIRDQPLWASFNGYSEFCSKSTGDGAVNINTRVVIKCPYTTPMLADNSNENQGFVPYSLNFGKGKMKGGSSQ
VPLSWRCKWYVMMYHQQEFMEAIVQSGPWAYKGDEKSAVLAMKYVFNWKWGGNPISGQVVRNPCKTSSSSREPR
SIQAVDPKRVAPPLVFHSWDYRRGLFGHASIKRMSEESATPTLFTGPAGKRPRRDTSVQQAEEDQQKESSSFRV
QRQLQPWIHSSQETQSSQEEMQAQGTVQEQLIQQLGEQRALRKQLEFLASQVLKVQAGQHIHPLLSSQA
>MN765788.1|Alpha|401_2629_2_fow
(SEQ ID NO: 2752)
MAWRWWWNRRPRRRWTRRRWRRLRTRRPRRPVRRRRRRRRVRRRRWGRRRGRRTFYRRRFKKRRRRRRKLTLKQ
WNPSTIRKCSIRGTVALVMCGHTKAGRNYALHSEDYVPSLISMGGSFSTTTWSLKALYDEFQKFHNRWSYPNTQ
LDLARFKGSKWTFYRDPKVDYIVTYYTVPPFKLNKLSSPFLHPGMMMQYKKKILVPSYQTKPKGKGKVSIWIKP
PVLFEDKWYTQQDLCPVNLLSLAVSAASFLHPFCPPESDNMCITFQVLQDFYYTQMGVTQMTGTTLTEADNNIF
KNHLYKQASYYQSLHTTGQLNATQKPANYSINDKIPQETWLSTTGPAGFKTGNNSIYGVPNYKPDYTKLEEIRK
WFFKQETTDNAIHGSYGKPVYNAVDYHIGKYSPIFLSPYRTNTQFSTAYRDITYNPNTDKGKGNHIWIQSVTKT
NTTLNSQCRCHIQDIPIWAAVTGYAEFCESELNSEVHNLYICCIICPYTKPKLYNNTDPTQGYVFYDTLFGDGK
MPTGTGLVPVWLQSRWYPRLIFQAQVLHDLYLTGPFSYKDDLKSFTISAKYNFKFLWGGTIIPEQVIKNPCKEQ
DSPFTFTGRQPRDLQVTDPQTMGPEWVFHTWDWRRGLFGENAIKRVSEQPSHDADYYAPPKKPRLFPPTDLQEQ
ESAFDFTPQELKILSEAPQQSEEEENQEEEQQLQQRELQLRLQEQFRIRHQLQHLFVQVLKTQAGLHINPLFLN
PV
>MN779270.1|Gamma|312_2300_3_fow (SAfiA)
(SEQ ID NO: 2753)
MPFWWRRRRKFWINNRFNYTKRRRYRKRWPRRRRRRRPYRRPIRRRRRKLRKVKRKKKSLIVRQWQPDSIRTCK
IIGQSAIVVGAEGKQMYCYTVNKLINVPPKTPYGGGFGVDQYTLKYLYEEYRFAQNIWTQSNVLKDLCRYINVK
LIFYRDNKTDFVLSYDRNPPFQLTKFTYPGAHPQQIMLQKHHKFILSQMTKPNGRLTKKLKIKPPKQMLSKWFF
SKQFCKYPLLSLKASALDLRHSYLGCCNENPQVFFYYLNHGYYIQTNWGAQSTTAYRPNSSVTETTYYKYKNEK
KNIHIKSSEYSESISYDKGYFQSSFLQTQCIYTSAAGDTCAAEKPLGIAIYNPVKDNGDGNMIYLVSTLANTWD
QPPKDSAILIQGVPIWLGLFGYLDYCRQIKADKTWLDSHILVMQSPAIFTYPNPGAGKWYCPLSQSFINGNGPF
NQPPTLLQKAKWFPQIQYQQEIINSFVESGPFVPKYANQTESNWELKYKYIFTFKWGGPQFHEPEIADPSKQEQ
YDVPDTFYQTIQIEDPEGQDPRSLIHDWDYRRGFVKERSLKRMSTYFSTHTDQQATSEEDIPKKKKRIGPQLTV
PQQKEEETLSCLLSLCKKDTFQETETQEDLQQLIKQQQEQQLLLKRNILQLIHKLKENQQMLQLHTGMLP
>MN778499.1|Gamma|300_2288_3_fow
(SEQ ID NO: 2754)
MPFWWPRRRRWWATTRFKRRKPRYPKRRRRRRYKRKYRRAPRRRRRRRRKVRRKKPNLIVRQWQPDSIVLCKIK
LFGSLVLGAEGTQYLCYTNEKGAWTPPQQPGGGGFGIETYSLSYLYDQYLLRRCIWTKSNDYKDLCRYLRCKIT
FFRHPKIDFVVNYARQPPFLLEKPTYMNCHPIALLLSQHKRVILSKRSDPKAKNRVTLIIKPPKQMITKWFFTK
QFALFDLLQIQAAACTLNYPKLGCCNENLIISIFYLNPQFFQDSQWAQYTTSPYKPYQTIDTQLTYHYKIGNQT
GQYKPEFLNKTGQQAYYESISLDKGWFSPKIMNTQYITQSGSETHLAAIPVGAARYNPEIDDGKGNEIYLVSVT
TGHYTKPSEDNLVFRGLPLWLMFFGLYSFIKKIKTASFLPLHMFVVKSEYILPKPKDPQKQYFPFVDLNFINGK
NPYNSYISSTQRQTWYPTCEHQVETINAFVKCGPFVPKLDNDRDSTWELPFKSTFYFKWGGPQMPNQNVSDPKD
KTSYIVPDTVQEGLQITNPLKQKAESLLHEWDYRRGLIKTKAFKRMLENLSIESDVYPSAETPRKRQRVENSLH
YQEEENQEIKTCLLSLCESSSSEKEEEEKNLLHLIQQQQQQQKQLKLQLLTLIKDLKEKQNALQLQTGVI
>MN776710.1|Gamma|288_2279_3_fow
(SEQ ID NO: 2755)
MPFWWRRRRKPWYGKWRFRRRRRYNAPRRKRKFYQRRRYRRTNRRRRKYKVRRKKRKTIAIRQWQPESITKCKI
IGFSNLVLGAEGTQYLCWTSQQEDYTQPKAPGGGGFGCELINLDWLYSEWVKHNNIWTKTNTNKDLVRYTGAYI
YFLRHPTIDFVIAYSLQPPFELTKFTYPDIQPQNLLLRKRKRIILSLASKPHGKRFVKVKIRPPKQMITKWFFQ
HQFSGYGLVQLSAAAATFRHPNIGPKSQNQMVTIYFLDTTIFNRPNWAQAQEDAWTPAGTHRTWTFEYQVKGQT
QPKTVQMPPSTPQDPYKTKYATSISRDYGWWQKAILNSTKVTIDGTMYSNRTVYSARYNPNEDTGQGNAVYLVS
LLQSKWTAPSDDRLVIVGQPLWMCMYGLYSYYQDLLKDKNFNTHYMFVVRSPAIKPITVSRQDFYPFIDWDFIN
GVLPWEEYLSEKIKATWYPQATFQTTTINALVESGPFVPRLINIPDSTWELVYKYKFFFKWGGPQTHDDHVQDP
KHQPDYDTPDTLHQTIQIGDPKKQTPESIIHEWDYRRGFITQTALKRMSENIQTDTDFQSDDSASPRKKRKVTK
KMPITSQKEENIKKCLLSLCQEETQETPENLQDLIQQQQQQQHLLKRNLLQLLTHLKQQQRYLSLQTGILD
>MN777204.1|Gamma|313_2331_1_fow
(SEQ ID NO: 2756)
MPFWWRRRRKFWRGRRRPFYKRITKRRRRRVRYRQRFRRPYRRRRRRRKHKVRRKRQKLPIYQWQPDSIRKCKI
KGVGTLILGAHGKQFVCYTDVQKKAPPPKAPGGGGFACEQFSLGGLYEQYKFRNNIWTASNINMDLCRYLGVRM
IFYRHPDIDFILNYDRQPPFVLDQLTYPLCHPQNLLLGKHKILLLSKATKPNGKLRKKVFIKPPKQMLNKWFFQ
EQFQSAPLLSIRASICSLQYPHIGCCMPNRTVTFFALNPGFYQQGNWAYDGGETNPYKPWPHIPKKLYFWDVAT
IPASWMEQEKAKHRVEIDLSTYSQSIDYQKGYFISKVLQAKLVTQDSTGISTIANTPLNIARYNPAIDTGKGNT
IWLHSNLTTAYTKPAVDKTVILEGLPLWMMLFGWLSWVQHVKGAPDFYLSYTLCVESPAIEIATKGETINPVIP
LDASFINGNAPYNQTISQKDLTHWYPDIYNQTEIINSIVTSGPYIPKLDNIKNSTWELHYYYQFFFKWGGPEIT
DQPVADPSKQPVYDALDKQQQGIQVRNPAKQKYATLMHPWDIRRGIITNKALKRMQQNLSIDSTFEAGSTPAKK
PRITGPCFTALQEIHQEERSCLQTLCKESTSQEAQEETDIQLLIAQQQQKQRELKYNLLKLISYMKEQQNMLKL
HTGMLN
>MN777719.1|Gamma|319_2301_1_fow
(SEQ ID NO: 2757)
MPFWWGRRNRFWYKNNYRRRAYRRKRKRWPKRRRRNFRRPTRRRRRRRRKQKVRKKKQTLTLKVWQPDTIRKCK
IKGTGIHIVGGNGTQYRCFTANRFDWTPPLMPGGGGFGVEKFTLQHLYNEHLLSNNIWTQSNKMLDLVRYTGCR
IKFYRHETIDFIASYNRNLPFNLEKYTYADSHPNSLLHSRHKKLIPSLKTRRSGKRYITMKIKPPKPVSNKWFF
QETFSSTPLFKLITSIIDLRYPHISQIGSNELVSLFCLNTDFYANAGWGNVHPPGTHSKWYVPYHNIAAITTVV
IKGRDTQITVDGSDYKKSVSYETGYFQPKLLQAEKIKNTAMENIPIQASRYNPTKDDGVGNKLWLVNVLNYSFA
PPKIDADLYIENLPLWQLVWGFTDFVKQTKKDSTFLNTYYAAFESKYLEPAVSTSKIYIPIDMSFIQGKGPYDS
YVSNYRKQNWYPILNAQLKTLNAIAQAGPFVPKLDNIFLSSWELYSNYTFYFKFGGSELPEPDTVNPQDQGTYV
IPTNIQKAIQVSNPQKQSPYTTLHSWDYRRGLITSSAFKRMCENQTTDTEFEPDTETIPKKKKKIQHGNQLQRA
EEETQEIQNCLLSLCEENTFQETPQTSLLELIQQQQQQQQQLKHNLLKLLSDLKLKQRSLQLQTGLIH

### Example 25: Evasion of Immune System

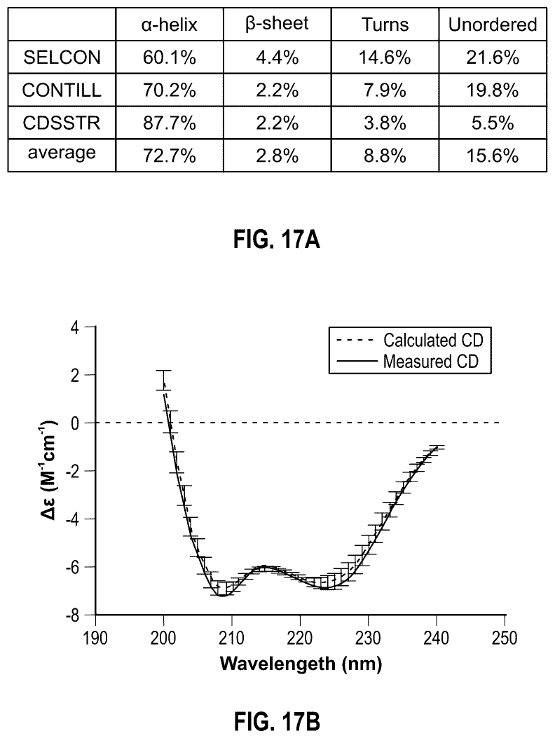

This Example describes the anellovirus C-terminal region and its relation to immune evasion. Absent from the Ring 10 structure is the majority of the C-terminal region (residues 545-672). Observed density for residues 545-562 showed that, were the C-terminal region present on the wild-type virus, the C-terminus would extend from the JR core toward the surface near the 3-fold axis between the spike domain crowns. The C-terminal region is semi-conserved between all anelloviruses, is predicted to be helical in nature, and has a glutamate-rich region (residues 636-640 on Ring 10) which could N-terminally cap a conserved helical motif. To determine if the C-terminal residues of Ring 10 would form a helical structure, circular dichroism experiments were performed, as described in Example 21, on the C-terminal peptides used to generate the aforementioned antibodies. Indeed, analysis with circular dichroism suggested that the C-terminal region (residues 635-672) is helical in solution, suggesting the C-terminal region would form a coiled-coil domain (FIGS. 17A and 17B), strongly suggesting a leucine-zipper like association of the helices perhaps due to the periodicity of hydrophobic residues. Electron microscopy of full-length Ring 10 ORF1 vs ORF1 which either had the C-terminus proteolytically removed or genetically removed suggested that particle formation is improved with the absence of the semi-conserved C-terminus (FIG. 14A-14C). Given that anelloviruses have evolved to evade the immune system, and that the C-terminal region is the immunodominant region of ORF1 and may be antagonistic to particle formation, the semi-conserved C-terminal region may be required for wild-type particle assembly but processed during viral particle maturation.

Despite anelloviruses constituting the majority of the human virome, their capsid structure was unknown. Determination of the Ring 10 structure as described by the examples included herein demonstrates that ORF1 encodes the capsid protein and that anelloviruses evolved a novel spike domain that extends around the 5-fold axis to form a crown structure. These crowns are capped with hypervariable P2 regions, which likely inhibits the development of antibodies against the better conserved spike P1 domain via steric hindrance. Analysis of the P1 surface revealed conserved residue patches that may have a receptor binding function.

The structure of the Ring 10 Betatorquevirus can be used to guide future anellovirus research. In particular, the diversity and immunological stealth of anelloviruses indicates that they could be used to deliver therapeutic genes to cell types not currently addressed by existing vectors, and that they may be less susceptible to pre-existing immunity or to the development of neutralizing antibodies following initial treatment. The availability of a capsid structure will help guide the design of anellovirus-based gene therapy vectors.

### Example 26. Grafting of HVR Helix Motif Between Anellovirus ORF1 Proteins

In this example, a peptide sequence in the structural hypervariable region (HVR) of the Ring 10 ORF1 protein was identified as a surface epitope. The surface epitope can, for example, be recognized by polyclonal antibodies to aid in analytical and purification development. Briefly, polyclonal antibodies (PAbs) were generated to Betatorquevirus strain Ring 10, guided by structural prediction of the viral particle formed by ORF1. The Ring 10 structure, determined as described herein, confirmed that a key helix (residues 352-361, having the amino acid sequence SPTQIEQIYT (SEQ ID NO: 2503)) is surface exposed in the viral particle.

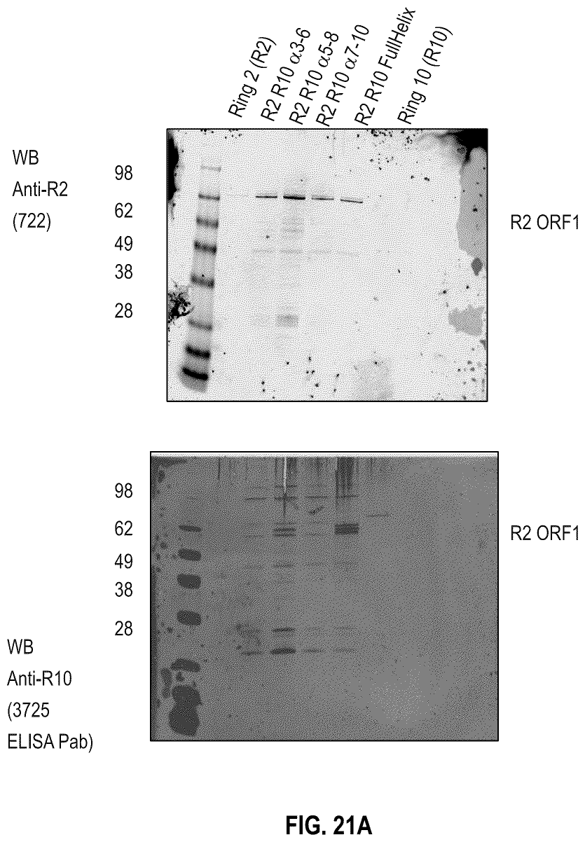

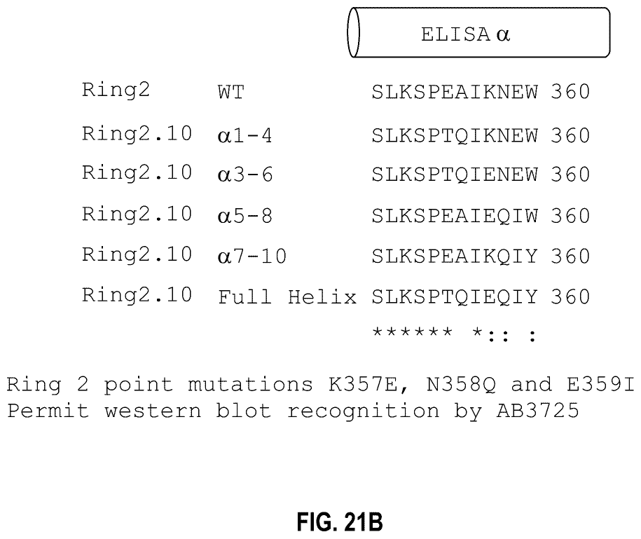

A PAb that recognizes Ring 10 HVR helix 352-361 PAb (AB3725) was shown to recognize Ring 10 anelloVLPs purified through SEC (FIG. 20). In addition, a series of point mutations at positions 357, 358, and 359 in in Ring 2 ORF1 to the corresponding residues of Ring 10 ORF1 (i.e., point mutations K357E, N358Q and E359I) allowed for recognition of the mutant Ring 2 ORF1s by AB3725 by western blot (FIG. 21A-21B). Lastly, a full helix swap of amino acids 357-359 from Ring 10 to Ring 2 ORF1 was recognized by AB3725 via western blot.

Without wishing to be bound by theory, it is contemplated that the HVR helix residues of Ring 10 (i.e., residues 352-361) can be grafted as ELISA epitopes into the ORF1 proteins of other Anelloviruses for purification or analytical purposes.

### Example 27: Production and Purification of AnelloVLPs

### Ring2 AnelloVLP:

A plasmid that encodes Ring2 ORF1 with a deletion of the C-terminus amino acids 611-666 (pRTx-2652; see also Table X1 construct listed as Ring2delCterm (A611-666) (7047)) was transfected into Expi293 cells. The cells were collected and resuspended in lysis buffer (50 mM Tris HCl, pH 8, 250 mM NaCl, 2 mM Magnesium Chloride). EDTA (0.5M) and protease inhibitor were added, and the cells were lysed using a microfluidizer. After lysing, protein inhibitor and triton X were added to the lysate and incubated for 30 minutes. The lysate was centrifuged at 12100 rpm and the supernatant was collected.

The lysate supernatant in 50 mM Tris HCl pH8, 250 mM NaCl was passed through a HiTrap heparin HP column (Cytiva) to separate 60mer VLPs from other smaller proteins from the mixture. The VLP fractions were dialyzed into Capto buffer (50 mM Tris HCl pH 8, 100 mM NaCl) overnight and incubated for 1.5 hrs with Capto400 resin at a 1:10 ratio of resin to protein. The preparation was spun down and supernatant collected.

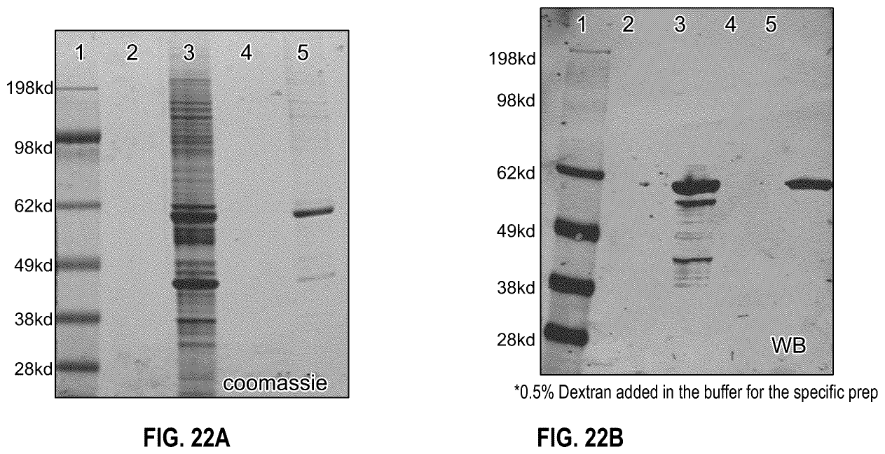

Samples were loaded onto a gel and Coomassie stained (FIG. 22A) or a Western blot was performed using a Ring2 HVR Linker primary antibody raised against amino acids 284-319 of the HVR region of Ring2 ORF1 and a goat-anti-rabbit secondary antibody (FIG. 22B). The lanes were as follows:
Lane 1: Ladder
Lane 2: Blank
Land 3: 2652 unpurified material
Lane 4: Blank
Lane 5: Ring2 AnelloVLP 2652 after Capto400 purification

Both gels show a relatively pure preparation of Ring2 VLPs after Capto400 purification (see lane 5).

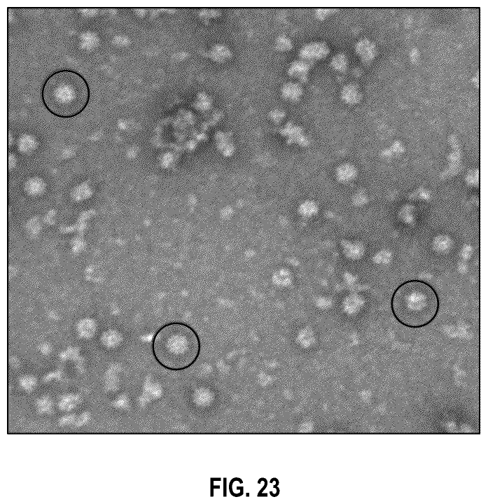

Electron microscopy of the preparation obtained after Capto400 purification confirmed Ring2 VLP formation at 65000× magnification (FIG. 23).

### Ring19 AnelloVLP:

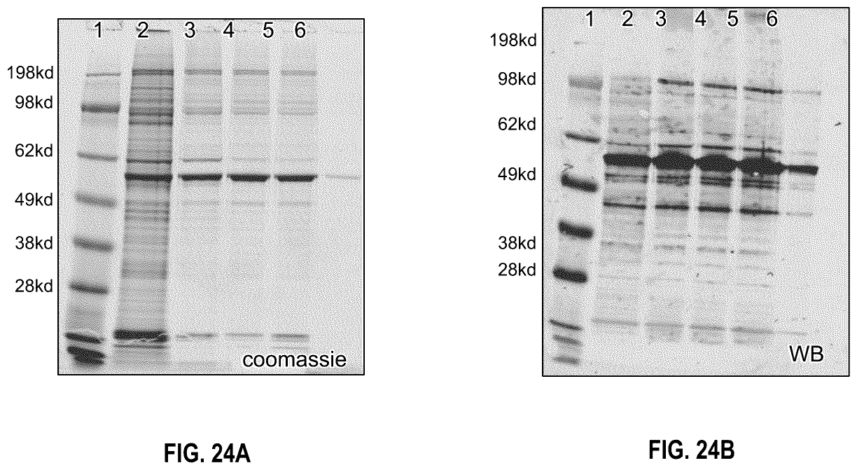

A plasmid that encodes Ring19 ORF1 with a deletion of the C-terminus amino acids 600-655 (pRTx-2814; see Table X1, construct Ring19delCterm (A600-655) (7120)) was similarly transfected as above into expi293 cells. The cells were collected and resuspended in lysis buffer (50 mM Tris HCl, pH 8, 250 mM NaCl, 2 mM Magnesium Chloride). EDTA (0.5M) and protease inhibitor were added, and the cells were lysed using a microfluidizer. After lysing, protein inhibitor and triton X were added to the lysate and incubated for 30 minutes. The lysate was centrifuged at 12100 rpm and the supernatant was collected. The supernatant was then purified through the HiTrap Heparin HP column and dialyzed into the Capto buffer as described above. The VLP preparation was then purified through a Capto400 column to further separate the VLPs from smaller proteins. Fractions “C12”, “D1”, and “D2” are different fractions collected during purification that showed peaks on the chromatogram. Samples from fractions C12, D1, and D2 were loaded onto a gel and Coomassie stained (FIG. 24A) or a Western blot was performed using a Ring19 HVR3 primary antibody that was raised against amino acids 342-352 of the Ring19 ORF1 HVR region (FIG. 24B). The lanes were as follows:
Lane 1: Ladder
Lane 2: Ring19 AnelloVLPs post heparin purification
Lane 3: Ring19 AnelloVLPs post dialysis
Lane 4: Ring19 AnelloVLPs capto400 C12 fraction
Lane 5: Ring19 AnelloVLPs capto400 D1 fraction
Lane 6: Ring19 AnelloVLPs capto400 D2 fraction

Both gels show Ring19 VLPs after capto400 purification.

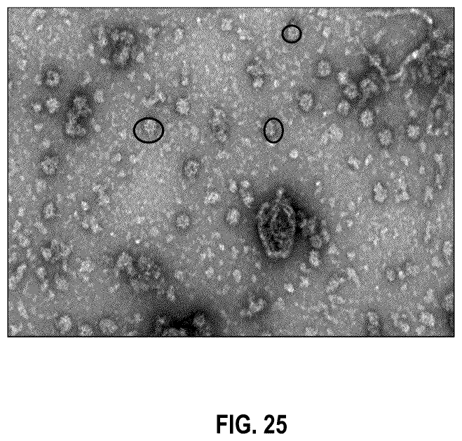

Electron microscopy of the preparation obtained after Capto400 purification confirmed Ring19 VLP formation at 65000× magnification (FIG. 25).

### Example 28: Conjugation of AnelloVLPs

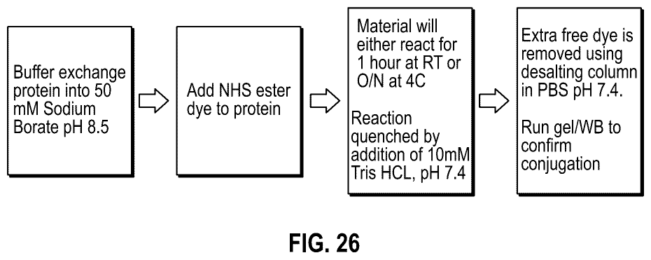

In this example, NHS ester moieties were conjugated via click chemistry to the surface lysines of the AnelloVLPs prepared according to the example above. The conjugation workflow is shown in FIG. 26.

The preparations of the AnelloVLPs prepared above were buffer exchanged into 50 mM sodium borate pH8.5 overnight and NHS ester 647-Alexa Fluor™ (“NHS Ester 647”) (Succinimidyl Ester), (ThermoFisher Catalog number: A20006) (for Ring2 AnelloVLPs) or EZ-Link™ NHS-Biotin (“NHS-Biotin”) (ThermoFisher Catalog number: 20217) (for Ring19 AnelloVLPs) were added in varying VLP: NHS Ester molar ratios and reacted for 1 hr at room temperature or overnight at 4° C. The reactions were stopped by addition of 10 mM Tris HCl, pH7.4. The preparations were further desalted in PBS pH7.4 to remove extra free NHS ester moieties. Gels or Western blots were run to confirm conjugation.

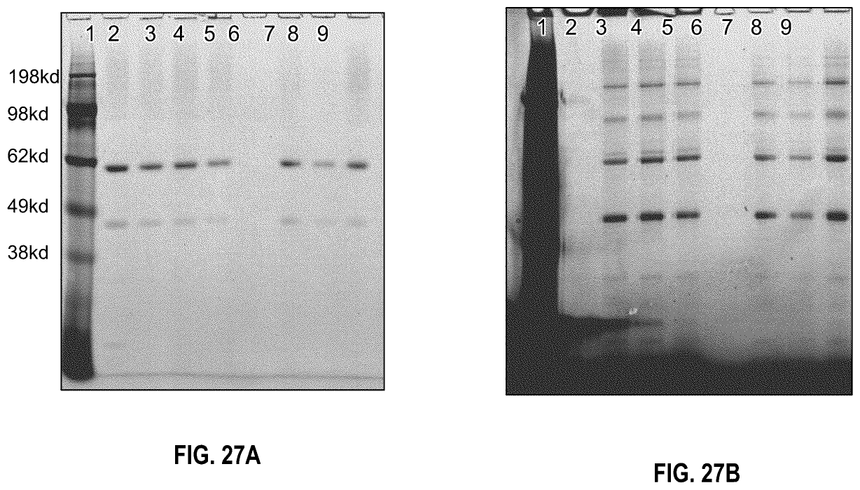

FIG. 27A shows a Coomassie stained gel and FIG. 27B shows a gel scanned under 520 nm of Ring2 AnelloVLP conjugated with NHS Ester 647. The lanes shown are as follows:
Lane 1: ladder
Lane 2: Ring2 AnelloVLP 2652 unlabeled
Lane 3: Ring2 AnelloVLP 2652 labeled at 1:5 VLP:NHS Ester 647 molar ratio, no desalt
Lane 4: Ring2 AnelloVLP 2652 labeled at 1:10 VLP:NHS Ester 647 molar ratio, no desalt
Lane 5: Ring2 AnelloVLP 2652 labeled at 1:20 VLP:NHS Ester 647 molar ratio, no desalt
Lane 6: Blank
Lane 7: Ring2 AnelloVLP 2652 labeled at 1:5 VLP:NHS Ester 647 molar ratio, desalted
Lane 8: Ring2 AnelloVLP 2652 labeled at 1:10 VLP:NHS Ester 647 molar ratio, desalted
Lane 9: Ring2 AnelloVLP 2652 labeled at 1:20 VLP:NHS Ester 647 molar ratio, desalted

The Coomassie-stained gel shows Ring2 AnelloVLPs at around 62 kD as expected in all protein-containing lanes. The gel scanned under 520 nm shows no visible band in lane 2 (unlabeled Ring2 AnelloVLP) while the other lanes containing labeled AnelloVPs show visible bands at about 62 kD, confirming the conjugation of Ring2 AnelloVLPS with NHS Ester 647.

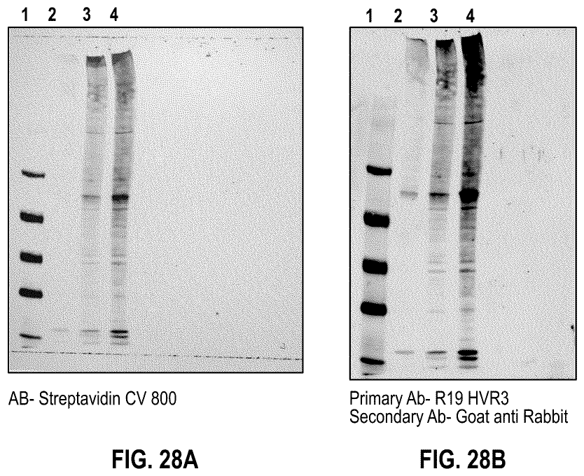

FIG. 28A shows a Western blot with a streptavidin CV 800 antibody [Licor Bioscience-Catalog number P/N: 926-32211] and FIG. 28B shows a Western blot with the R19 HVR3 primary antibody described above and a goat-anti rabbit secondary antibody. The lanes shown are as follows:
Lane 1: Ladder
Lane 2: Ring19 AnelloVLP unlabeled
Lane 3: Ring19 AnellovLP labeled at 1:5 VLP:NHS Ester biotin molar ratio [desalted]
Lane 4: Ring19 AnellovLP labeled at 1:10 VLP:NHS Ester biotin molar ratio [desalted]

The Western blot using the Ring19 specific antibody shows visible bands at around 62 kD in both the unlabeled and labeled Ring19 AnelloVLP samples. The Western blot with streptavidin shows a band at around 62 kD with Ring19 AnelloVLPs labeled with NHS Ester biotin while the unlabeled Ring19 AnelloVLP shows no visible band, confirming the conjugation of Ring19 AnelloVLPs with NHS Ester biotin.

### Example 29: VLP Conjugation to SARS-CoV-2 RBD Peptide

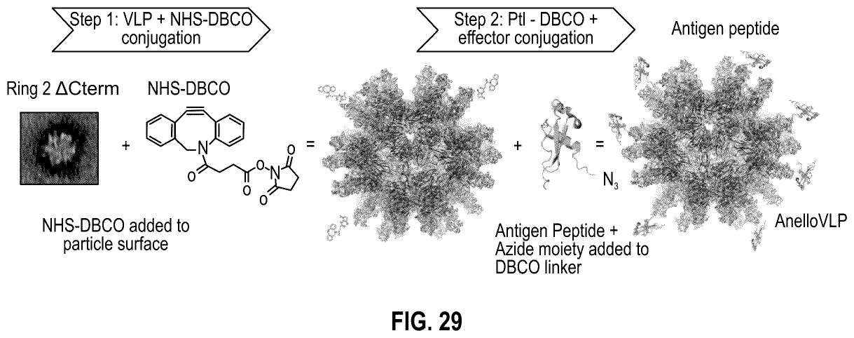

In this Example, a His-Tag recombinant receptor binding domain (RBD) comprising amino acids 319-541 from SARS-CoV-2 coronavirus spike protein (ThermoFisher Catalog #RP-87704) (referred to in this example as RBD) was conjugated to the surface of AnelloVLPs. As shown in FIG. 29, the conjugation involved a first step of conjugating NHS-DBCO to the surface ORF1 of anelloVLPs. Azide-labeled RBD effectors were then conjugated to the anelloVLPs via the DBCO linker. This approach can be generalized to attach azide-labeled effectors (e.g., antigenic peptides and proteins) to anelloVLPs with DBCO linkers on their surfaces.

First, RBD was labeled with either DBCO or Azide to confirm that RBD could be labeled for future conjugation to AnelloVLPs. In brief, 100 μg of the His-tagged recombinant RBD (ThermoFisher Catalog #RP-87704) was resuspended in 1 ml of sodium borate buffer (pH=8.5) to final concentration of 0.1 mg/ml. The RBD amino acids from 319-541 contains 11 lysines that could potentially be labeled by NHS. Thus, in one condition, 2 μl of DBCO-PEG4-NHS 1 mg/ml was added to 200 μl of 0.1 mg/ml RBD and incubated overnight at 4° C. (2 μg DBCO-PEG4-NHS: 20 μg RBD). In a second condition, 2 μl of Azide-PEG4-NHS at 1 mg/ml was added to 200 μl of 0.1 mg/ml RBD and then incubated overnight at 4° C. (2 μg Azide-PEG4-NHS: 20 μg RBD). The next day, a zeba desalting column was prepared by opening the bottom of a column and placing it into a microfuge tube, spinning at 1500 g for 1 min at 4° C., adding 400 μl of Borate buffer (50 mM sodium borate) and spinning at 1500 g for one minute and then discarding the flow-through. This process is then repeated 3-4 times and the column is then placed into a fresh microfuge tube.

Unreacted DBCO/Azide was desalted out of the samples by running through a desalting column for two minutes at 1500 g and at 4° C. Samples were then split as follows:
a. 100 μl RBD-PEG4-DBCO
b. 100 μl RBD-PEG4-DBCO+CaIFlour488
c. 100 μl RBD-PEG4-Azide
d. 100 μl RBD-PEG4-Azide+Alexa488 sDIBO

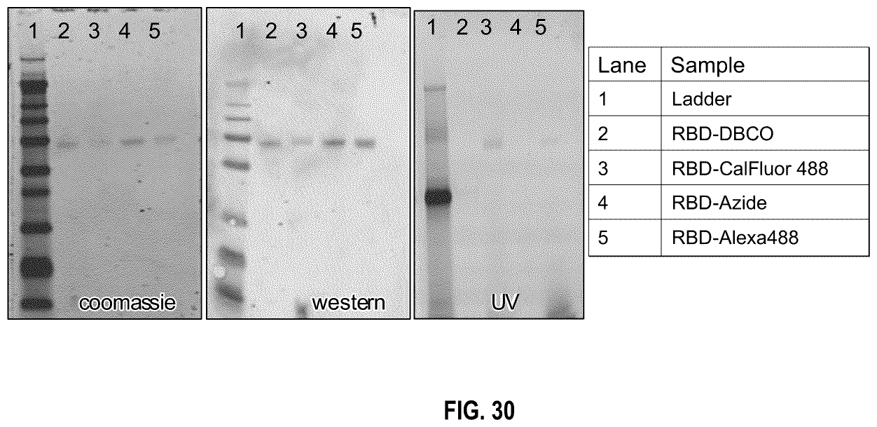

RBD-PEG4-DBCO+CaIFlour488 and RBD-PEG4-Azide+Alexa488 sDIBO were each made by adding 1 μl of CaIFluor488 Azide/Clicklt Alexa488 sDIBO to the RBDs. The mixture was incubated for 30 minutes, and this process was then repeated three times. RBD-PEG4-DBCO+CaIFlour488 and RBD-PEG4-Azide+Alexa488 sDIBO samples were then desalted as described. A western blot was then run to determine whether RBD labeling occurred. As shown in FIG. 30, labeling of RBD was confirmed with both DBCO and azide for future VLP conjugation. This demonstrated that a linker could be conjugated to the RBD.

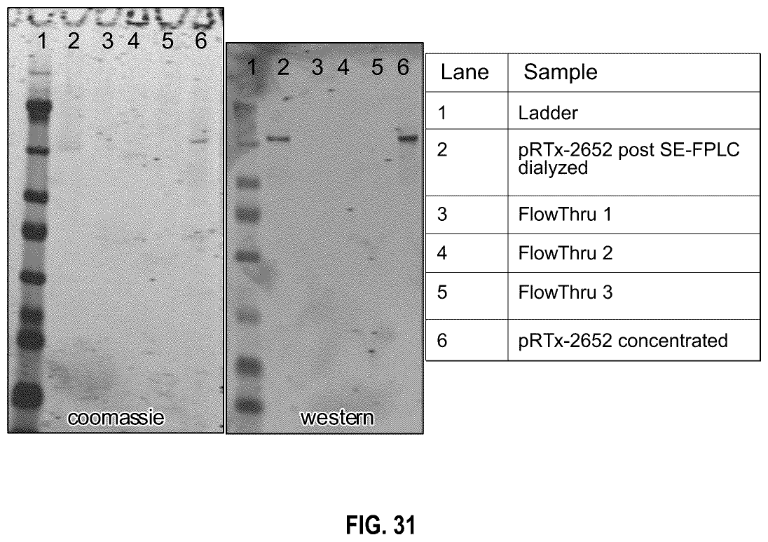

To conjugate RBD to a Ring2 VLP, a fraction block of Ring2 ORF1 with a C-terminal deletion of amino acids 611-666 (pRTx-2652; corresponding to Ring2delCterm (A611-666) (7047) as described herein) in TBS was produced using SE-FPLC. Fractions were pooled and dialyzed into 2 L of PBS+0.01% poloxamer at 4° C. After 3 hours, buffer was refreshed. This was then repeated twice more and left overnight at 4° C. Approximately 30 ml of the post-SE-FPLC solution was concentrated to approximately 1.5 ml final volume. A Western blot was run to confirm that the sample was retained (FIG. 31). The next day, approximately 1.5 ml of SE-FPLC pRTx-2652 was concentrated down to 220 μl.

1 μl NHS-PEG4-DBCO was added to 100 μl SE-FLPC pRTx-2652 samples generated as described above. The samples were then incubated for 1 hour at room temperature. 3 μl 1M Tris pH 8 was added, and the samples were desalted. RBD was labeled with NHS-PEG4-Azide by resuspending RBD in 200 μl borate buffer (0.5 mg/mL final) and 1 μl NHS-PEG4-Azide for 1 hour at room temperature. 3 μl 1M Tris pH 8 was then added and the RBD sample was desalted.

7.5 μl of the RBD peptide was then added to 30 μl of DBCO-2652 to produce a final peptide concentration 0.1 mg/ml. The sample was then incubated at 37° C. for 2.5 hours and conjugation was assessed by Coomassie staining and Western blot with an anti-Ring2 ORF1 antibody. As shown in FIG. 32, a 90 kDa band corresponding to the conjugation of RBD (26 kDa) to pRTx-2652 anelloVLPs (˜62 kDa) was observed.

### Example 30: Ring19-eGFP Anellovector Transduces Eye Tissue In Vivo

In this example, eGFP was transduced into mouse eye tissue in vivo by subretinal injection of Ring19-eGFP Anellovector, demonstrating the utility of the anellovectors for ocular delivery of exogenous effectors in vivo.

### Methods and Materials

### Vectors:

AAV2-fCMV-eGFP was prepared by PackGene Biotech Inc. and was diluted in sterile 1×PBS. Ring19-fCMV-eGFP was prepared as described below.

### Parental Cell Culture

MOLT-4 cells were obtained from the National Cancer Institute. Cells were scaled-up and maintained in suspension culture in complete growth medium (Gibco's RPMI 1640 with 10% fetal bovine serum [FBS], supplemented with 1 mM sodium pyruvate, Pluronic F-68 [0.1%], and 2 mM L-glutamine) at 37° C. with 5% CO₂. Cells were seeded into shake flasks (2-L, flat-bottomed, Erlenmeyer flask), each with a working volume of 800 mL, at a density of 0.1 E+06 viable cells/mL and cultured in an orbital shaker (New Brunswick Innova 2100, 19-mm circular orbit) at 37° C. and 100 rpm with >85% relative humidity (RH) for 4 days.

### Transfection of MOLT-4 Cells

MOLT-4 cells were transfected with the three in-house designed plasmids (pRTx-2847; pRTx-2848; pRTx-3525) via electroporation. For electroporation at 200 mL scale, 10⁸ pelleted cells were resuspended in Opti-MEM I Reduced Serum Medium. 120 μg of the plasmids (study #1 material) and 140 μg (study #2 material) was added to the resuspended cells and electroporated using a MaxCyte STx electroporator and R-1000 electroporator assembly (MaxCyte catalog #EROO1M1-10). Electroporated cells were then transferred to a flask containing pre-warmed complete growth medium. Transfected cells were allowed to incubate at 37° C. with 5% CO₂ and harvested 72 hours post-electroporation.

### Cell Lysis

Cell pellets were resuspended in lysis buffer containing 50 mM Tris pH 8.0, 0.5% Triton-X100, 100 mM NaCl, 100× Halt protease inhibitor cocktail (Thermo Fisher Scientific catalog #78439), and 100 of mSAN nuclease (ArcticZyme catolog #NC1920045). The cell lysates were clarified by centrifugation at 12,500×g for 30 minutes at 4° C.

### Isopycnic Centrifugation

To prepare iodixanol linear gradients, 13 mL of 60% OptiPrep (Sigma-Aldrich catalog #D1556) was overlaid with 13 mL of 20% OptiPrep in 26.3-mL polycarbonate tubes, which were then spun at a 46-degree angle and a speed of 20 rpm for 16 minutes using Gradient Master (BioComp). Following the generation of the iodixanol linear gradient, 2 mL of iodixanol were removed from the top of each gradient, and 2 mL of clarified lysate was added on top of the gradient. The sample-containing tubes were spun at 347,000×g and 20° C. for 3 hours using Type 70 Ti rotor (Beckman Coulter). 1-mL fractions were collected from the top of the tubes and transferred to a 96-well 2.2 ml capacity plate. The refractive index of each fraction was measured using Refracto handheld refractometer (Mettler Toledo) to calculate density. Each fraction was then subjected to DNase-protected qPCR assay as described below.

### DNase-Protection Assay

5 μl of each sample to be titered was incubated with 20 U of DNAse I endonuclease (Thermo Fisher Scientific catalog #18047019) in a 20 μl reaction. The reaction was incubated at 37° C. for 30 minutes. Following DNase-treatment, each sample was subjected to Proteinase K (Fisher Scientific catalog #FEREOO491) and proteinase K buffer (1% SDS, 0.1M EDTA, 0.1M Tris pH 8.0, 0.1% Pluronic F-68). The reaction was incubated at 37° C. for 30 minutes, followed by proteinase K inactivation at 95° C. for 15 minutes. 4 μl of the 1:10 diluted DNase reaction was subjected to qPCR analysis in a 20-μl reaction using TaqMan Fast Universal PCR Master Mix (Thermo Fisher Scientific catalog #44-449-63) according to the manufacturer's protocol. Primer and probe sequences are listed in Table AA1.

### Concentration of Material

Fractions of interest were determined based on the viral titer and density measurements. The pooled iodixanol fractions were then diluted 1:50 in formulation buffer (1×DPBS, 0.001% Pluronic F-68) and concentrated using Vivaspin 20 100K MWCO centrifugal filter units (Fisher Scientific catalog #1455810).

### DNase-Protection Assay on Final Material

5 μl of the sample to be titered was incubated with 20 U of DNAse I endonuclease (Thermo Fisher Scientific catalog #18047019) in a 20 μl reaction. The reaction was incubated at 37° C. for 30 minutes. Following DNase-treatment, each sample was subjected to Proteinase K (Fisher Scientific catalog #FEREOO491) and proteinase K buffer (1% SDS, 0.1M EDTA, 0.1M Tris pH 8.0, 0.1% Pluronic F-68). The reaction was incubated at 37° C. for 30 minutes, followed by proteinase K inactivation at 95° C. for 15 minutes. 4 μl of the 1:10 diluted DNase reaction was subjected to qPCR analysis in a 20-μl reaction using TaqMan Fast Universal PCR Master Mix (Thermo Fisher Scientific catalog #44-449-63) according to the manufacturer's protocol. Primer and probe sequences are listed in Table AA1.

TABLE AA1
Primer and probes designed to quantify eGFP and WT R19

| Target | Label | Sequence (5′→3′) |
|---|---|---|
| eGFP | Forward Primer | GAACCGCATCGAGCTGAA (SEQ ID NO: |
| | | 2690) |
| | Reverse Primer | TGCTTGTCGGCCATGATATAG (SEQ ID |
| | | NO: 2691) |
| | Probe (FAM) | ATCGACTTCAAGGAGGACGGCAAC |
| | | (SEQ ID NO: 2692) |
| WT R19 | Forward Primer | GGATTTTGGGAGGGTCACTC (SEQ ID |
| | | NO: 2693) |
| | Reverse Primer | TACAGTTCCTGGACCTGTGT (SEQ ID |
| | | NO: 2694) |
| | Probe (FAM) | ACACTGGTACCCTAAAAATAGATTTCA |
| | | (SEQ ID NO: 2695) |

### Endotoxin Test on Final Material

2.75 μl of the sample was diluted 1:40 in formulation buffer (1×DPBS, 0.001% Pluronic F-68) and the sample was subjected to the LAL detection test (Charles River) according to the manufacturer's protocol.

### SDS-PAGE and Silver Stain on Final Material

2 μl of the sample was diluted 1:10 in formulation buffer (1×DPBS, 0.001% Pluronic F-68) and the sample was mixed 1:1 with loading dye and Bolt sample reducing agent (Thermo Fisher Scientific catalog #B0009), followed by boiling at 95° C. for 5 minutes. Proteins were separated on Bolt 4-12% Bis-Tris gel in 1× Bolt MOPS SDS running buffer (Thermo Fisher Scientific catalog #B0001). Separated proteins were stained using SilverQuest Silver Staining Kit (ThermoFisher Scientific catalog #LC6070) according to the manufacturers protocol. The developed silver stain was visualized using Chemidoc Imaging System (BioRad).

### SDS-PAGE and Coomassie Stain on Final Material

2 μl of the sample was diluted 1:10 in formulation buffer (1×DPBS, 0.001% Pluronic F-68) and the sample was mixed 1:1 with loading dye and Bolt sample reducing agent (Thermo Fisher Scientific catalog #B0009), followed by boiling at 95° C. for 5 minutes. Proteins were separated on Bolt 4-12% Bis-Tris gel in 1× Bolt MOPS SDS running buffer (Thermo Fisher Scientific catalog #B0001). Separated proteins were washed 3 times with diH2O and incubated with Coomassie Brilliant Blue G-250 Protein Stain (Cepham Life Sciences catalog #10491). Following the stain, the gel was washed three times with diH2O. The stained gel was visualized using Chemidoc Imaging System (BioRad).

### Subretinal Injections

Mouse pupils were first dilated with one to two drops of 1% tropicamide/2.5% phenylephrine HCl (Tropi-Phen, Pine Pharmaceuticals). The mouse was subsequently anesthetized using an intraperitoneal injection of a ketamine/xylazine cocktail (Patterson Veterinary Supply, Inc.) cocktail (100/10 mg/kg). One or two drops of 0.5% proparacaine (McKesson Medical-Surgical Inc.) were applied to the eye. An incision approximately 0.5 mm in length was made with a micro scalpel 1 mm posterior to the nasal limbus. A 33-g blunt-ended needle on a 5-μl Hamilton syringe was inserted through the scleral incision, posterior to the lens, toward the temporal retina until resistance was felt. 1 μl of either 1×PBS, virus, or vector containing 0.1% of sodium fluorescein (AK-Fluor 10%, Akorn) was then injected slowly into the subretinal space. The eye was examined and the success of the subretinal injection was confirmed by visualizing the fluorescein-containing bleb through the dilated pupil with a Leica M620 TTS ophthalmic surgical microscope (Leica Microsystems, Inc.). Eyes with significant hemorrhage or leakage of vector solution from the subretinal space into the vitreous were excluded from the study. After the procedure, 0.3% tobramycin ophthalmic ointment (Tobrex, McKesson Medical-Surgical Inc.) was applied to each treated eye and the mouse was allowed to recover from the anesthesia prior to being returned to its cage in the housing room.

### Tissue Harvest for Flatmount Analysis

Mouse eyes were dissected at the 21 days following subretinal injections (n=2). After enucleation, whole eyes were placed into 4% paraformaldehyde (PFA) solution for fixation. The fixed eyes were transferred to 1×PBS after 2 hours.

The fixed eye was placed into a petri dish with 1×PBS under the Leica M80 stereomicroscope (Leica Microsystems Inc.). The cornea and lens were removed and discarded. The neural retina was separated from the posterior eye cup (PEC) and discarded. 8 radial cuts were evenly placed throughout the PEC, perpendicular to the optic nerve head. The PEC was then moved onto a glass microscope slide and arranged in a flat, even position. 1-2 drops of mounting media (ProLong Glass Antifade Mountant, Invitrogen) were dispensed over the tissue. A glass cover slip was placed over the mountant and slight pressure was applied to remove bubbles and further flatten the tissue. The slides were left for a minimum of 2 hours to cure before imaging.

### Flatmount Imaging

Once cured, images of the PEC flatmount slides were collected from each group using the EVOS M7000 microscope (Life Technologies Corporation). Images were captured in the green channel (EVOS Light Cube, GFP 2.0, Life Technologies Corporation) and the red channel (EVOS Light Cube, Texas Red 2.0, Life Technologies Corporation) at 20× magnifications, utilizing congruent exposures for each image.

### Tissue Harvest for DNA and RNA Extraction

Mouse eyes were dissected at 21 (n=8 for experiment 1 and n=8 for experiment 2) or 49 (n=8 for experiment 2) days following subretinal injections. After enucleation, the retina and PEC were separated and processed individually. These tissues were collected in 2 mL reinforced tubes (SPEX Sample Prep) containing 5 mm stainless steel beads (Qiagen, LLC) and flash-frozen immediately. They were stored at −80° C. until ready for homogenization.

### DNA Isolation

Frozen tissue samples were lysed with automated tissue homogenizer (Geno/Grinder SPEX Sample Prep) in Buffer ATL (Qiagen, USA) and proteinase K (Qiagen, USA) at 1250 rpm for two 30 second rounds. Homogenized tissues were digested on a heat block at 56 C for approximately 4 hours. Genomic DNA was precipitated with Buffer AL (Qiagen, USA) and ethanol, then isolated with Qiagen DNeasy 96 Blood & Tissue Kit. Isolated DNA was quantified using NanoDrop 8000 Spectrophotometer (Thermofisher, USA).

qPCR

Genomic DNA was assayed by qPCR on the QuantStudio 5 Real-Time PCR System (Thermo Fisher, USA) using TaqMan Gene Expression Master Mix (Thermofisher, USA). The sequence detection primers and FAM custom probes that were used in this study were synthesized by Integrated DNA Technologies, USA. eGFP sequences and wild-type (WT) Ring19 primer/probe sequences are included in Table AA1. Mouse GAPDH was used as the endogenous control in duplicates. All reactions including the DNA samples and different dilutions of a known quantity of the linearized eGFP and R19 plasmid standards were run in triplicate on the same plate. The standard curve method was used to calculate the amount of viral/vector DNA, which was normalized with the total amount of genomic DNA for each sample (quantified using nanodrop as described above).

### RNA Isolation

Frozen tissue samples were lysed with an automated tissue homogenizer (Geno/Grinder SPEX Sample Prep, USA) in QIAzol lysis reagent (Qiagen, USA) at 1250 rpm for two 30 second rounds. RNA was isolated in aqueous phase by addition of Phenol Chloroform (Thermofisher, USA) and centrifugation at 6000 rpm for 15 minutes at 4 C. The upper aqueous phase was transferred into a fresh S-block (Qiagen, USA). RNA was then precipitated with the addition of 1 volume of 70% Ethanol and isolated with the Qiagen RNeasy 96 kit. RNA concentration was quantified via Qubit RNA High Sensitivity Assay Kit (Thermofisher, USA).

### RT-qPCR

Isolated RNA was reverse transcribed into cDNA using Thermofisher SuperScript IV VILO Master Mix with ezDNase according to the manufacturer's protocol, with additional reactions included for No Reverse Transcriptase (NRT) controls. cDNA was then diluted with nuclease-free water to ensure enough volume to run all qPCR assay reactions necessary. cDNA was assayed by qPCR on the QuantStudio 5 Real-Time PCR System (Thermo Fisher, USA) using TaqMan Gene Expression Master Mix (Thermofisher, USA). The sequence detection primers and FAM custom probes that were used in this study were synthesized by Integrated DNA Technologies and can be found in Table AA1. Mouse GAPDH was used as the endogenous control in duplicates (ThermoFisher, USA). All reactions including the cDNA samples and different dilutions of a known quantity of the linearized eGFP plasmid standards were run in triplicate on the same plate. The standard curve method was used to calculate the quantities of mRNA copies and was normalized to both the no-RT signal and the total amount of RNA input into the RT reaction.

### One-Step RT-ddPCR

RNA was diluted in nuclease-free water and combined with the reagents from the One Step RT-ddPCR Advanced Kit for Probes (Bio-Rad, USA; Catalog #1864022) and eGFP primer/probe set with final primer concentrations of 900 nM and probe concentrations of 250 nM to measure transgene expression. After the RT-ddPCR reaction setup, each reaction was converted to droplets using the Automated Droplet Generator (Bio-Rad, USA) according to the manufacturer's instructions. The droplets were then subjected to endpoint PCR thermocycling with the following cycling conditions: 1 cycle of 48 C for 1 hour for reverse transcription followed by 1 cycle of 95 C for 10 mins; 40 cycles of 95 C for 30 sec, 60 C for 1 min; and 1 cycle of 98 C for 10 min and finally a 4 C hold. The cycled plate was then transferred to the QX200 Droplet Reader (Bio-Rad, USA) and analyzed using QX Manager Software (Bio-Rad, USA).

### Results

### Production and Infectivity of Ring19-eGFP Anellovector

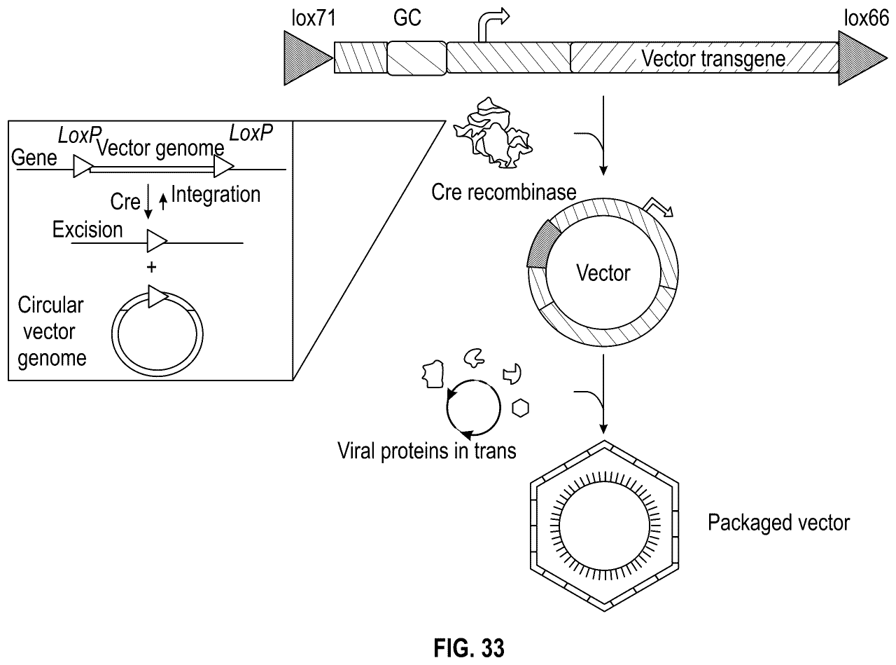

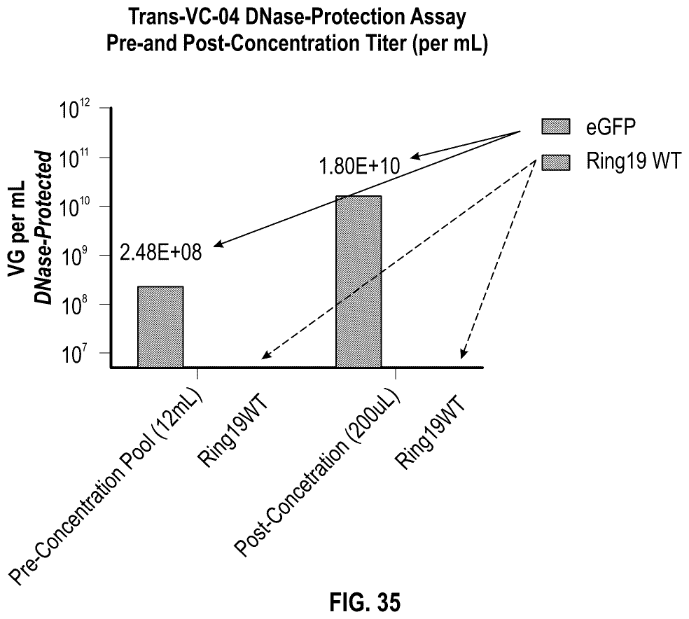

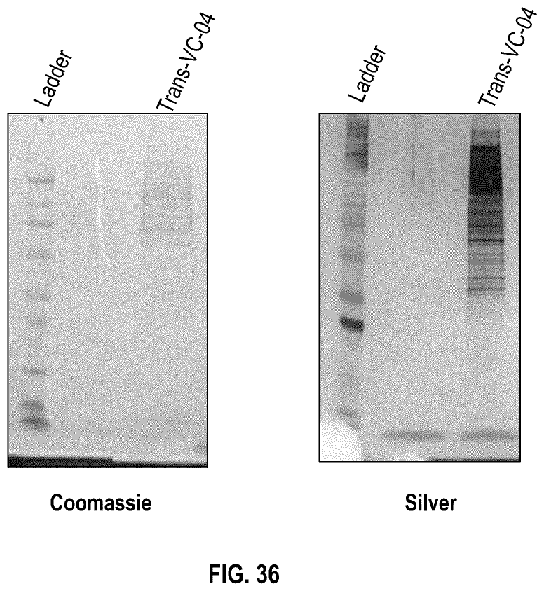

An exemplary Cre-loxp based vector system was employed to produce Ring19-eGFP Anellovectors. FIG. 33 shows a schematic of this system. A DNase protection assay followed by qPCR was utilized to detect both Ring19-eGFP and wild type (WT) Ring19 genetic material. FIG. 34 shows a plot of DNase protected genome copies for each fraction collected for both Ring19-eGFP and WT Ring19. WT Ring19 genomes were below the limit of quantification (LOQ) in the shown fractions (FIG. 34 and FIG. 35). 12 mL of the pooled viral particles were then concentrated from 2.48×10⁸ viral genomes (vg) per mL to 200 uL at a concentration of 1.8×10¹⁰ vg per mL (FIG. 35) for use in the first subretinal injection experiment (Table AA2). 1 uL of solution was injected per eye. WT Ring19 genomes were below the LOQ by qPCR after concentration (FIG. 35). Viral proteins were run on an SDS-PAGE gel with Coomassie blue and silver staining (FIG. 36).

TABLE AA2
Experimental design for subretinal injection experiment 1 using virus batch 1

| Group | Treatment Day 0 | Dose/eye (vg) | Route (volume) | N (eyes) | Terminal Day |
|---|---|---|---|---|---|
| 1 | PBS | 0 | Subretinal (1 ul) | 10 | 21 |
| 2 | R19-fCMV-eGFP | 1.8e+7 | Subretinal (1 ul) | 10 | 21 |
| 3 | AAV2-fCMV-eGFP, dose-matched (DM) | 1.8e+7 | Subretinal (1 ul) | 10 | 21 |
| 4 | AAV2-fCMV-eGFP, high dose (HD) | 1.0e+9 | Subretinal (1 ul) | 10 | 21 |

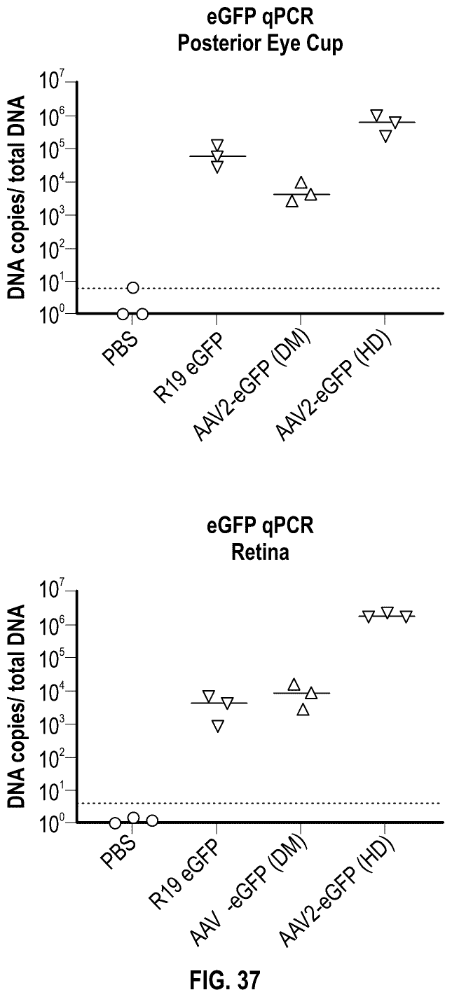

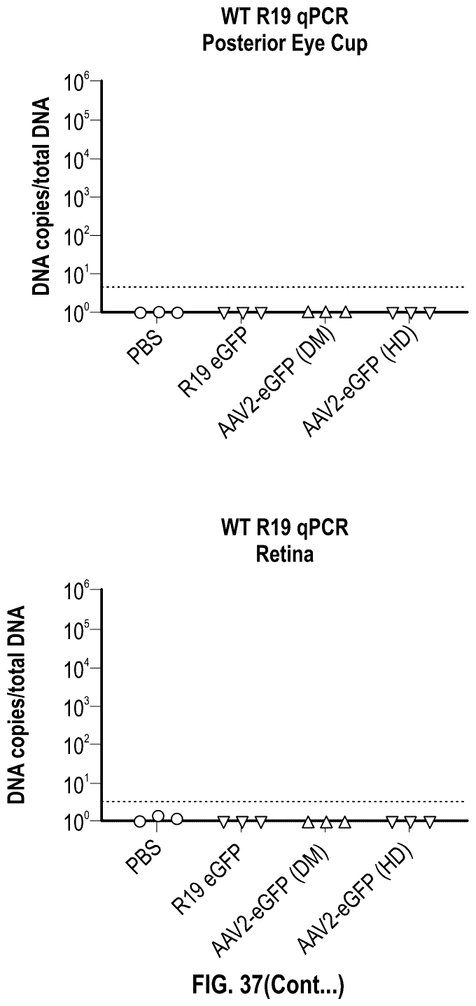

The prepared virus was then injected into mice subretinally to target infection of eye tissue as shown in experiment 1 (Table AA2). Mice were injected with PBS, Ring19-eGFP, dose matched (DM) AAV2-eGFP, or high dose (HD) AAV2-eGFP. 21 days after injection, the posterior eye cup (PEC) and retina were collected and processed separately. Only Ring19-eGFP genomes were detected by qPCR in the PEC and the retina (FIG. 37, first page). The level of eGFP genomic DNA was significantly greater in the PEC infected with Ring19-eGFP compared to PEC infected with dose-matched AAV2-eGFP (FIG. 37, top of first page) and similar levesl of eGFP genomic DNA was detected in the retina infected with Ring10-eGFP compared to retina infected with dose-matched AAV2-eGFP (FIG. 37, bottom of first page). Further, WT Ring19 genomes were not detected by qPCR in either tissue at day 21 (FIG. 37, second page), demonstrating that viral vector genomes can be produced efficiently without WT Ring19 contamination.

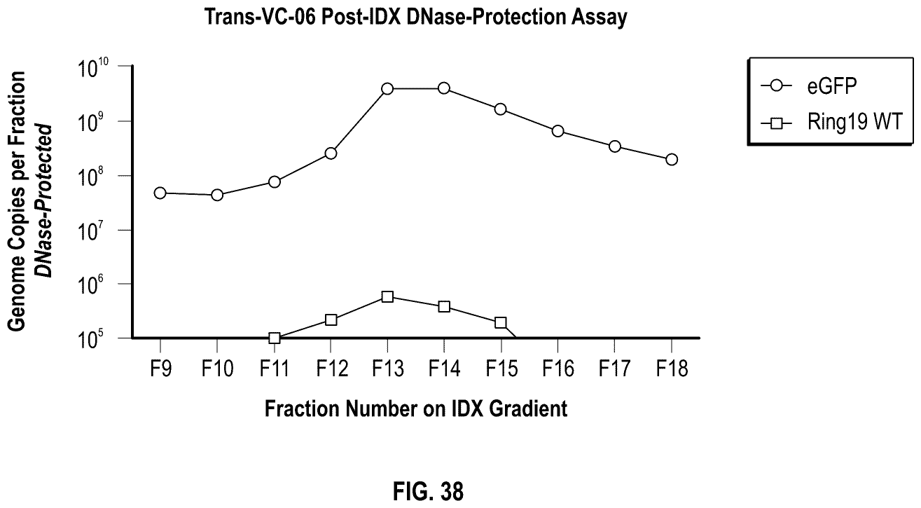

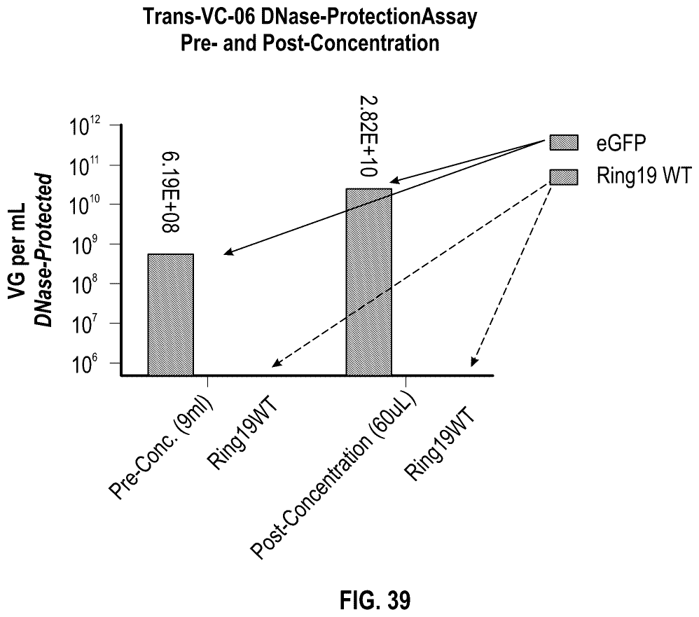

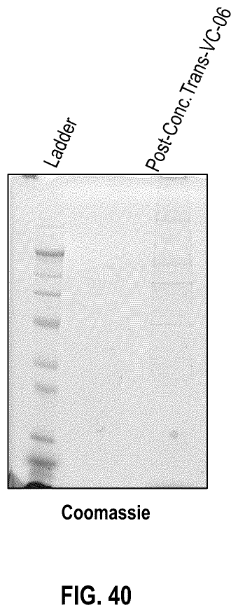

FIG. 38 shows the results of the DNase protection assay for a second batch of virus used in a second subretinal injection experiment summarized in Table AA3. Similarly to batch 1, Ring19-eGFP was found at high levels in several iodixanol fractions (FIG. 38). The WT Ring19 signal observed in some of the fractions is likely due to plasmid carryover producing trace levels of WT Ring19 signal, or contamination during qPCR, since the pre- and post-concentration signals were below the LOQ after the virus preps were pooled and filtered (FIG. 39). 9 mL of the pooled virus was concentrated from 6.19×10⁸ vg per mL to 60 uL at a concentration of 2.82×10¹⁰ vg per mL (FIG. 39), then viral proteins were assessed on a coomassie blue stained SDS-PAGE gel (FIG. 40).

The subretinal injection experiment was then repeated with the second batch of virus, including tissue collection at 21 days similar to experiment 1 and extending a second collection to 49 days as outlined in Table AA3.

TABLE AA3
Experimental design for subretinal injection experiment 2 using virus batch 2

| Group | Treatment Day 0 | Dose/eye (vg) | Route (volume) | N (eyes) | Terminal Day |
|---|---|---|---|---|---|
| 1 | PBS | 0 | Subretinal (1 ul) | 8, 8 | 21, 49 |
| 2 | R19-fCMV-eGFP | 2.5e+7 | Subretinal (1 ul) | 8, 8 | 21, 49 |
| 3 | AAV2-fCMV-eGFP, dose-matched (DM) | 2.5e+7 | Subretinal (1 ul) | 8, 8 | 21, 49 |
| 4 | AAV2-fCMV-eGFP, high dose (HD) | 1.0e+9 | Subretinal (1 ul) | 8, 8 | 21, 49 |

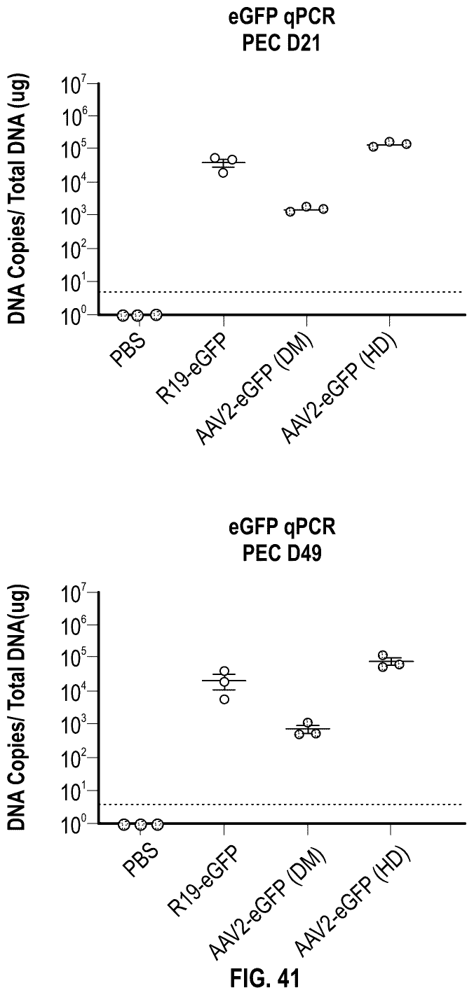

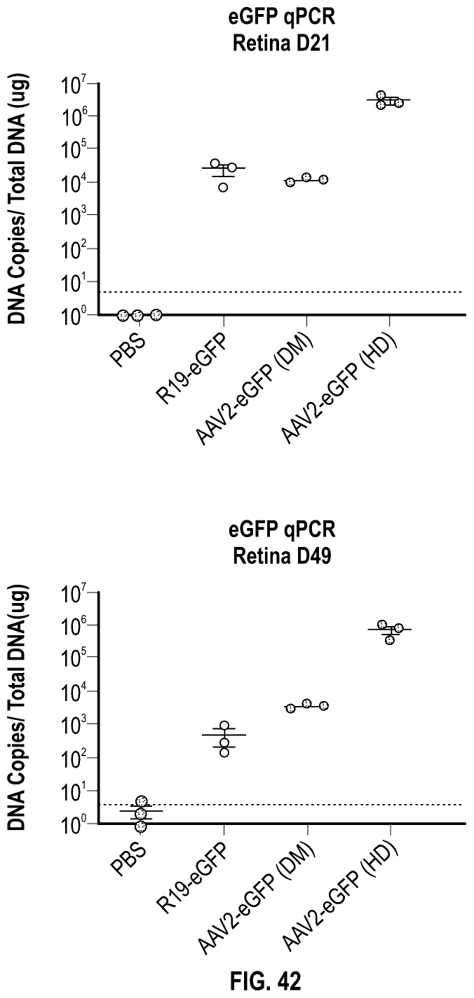

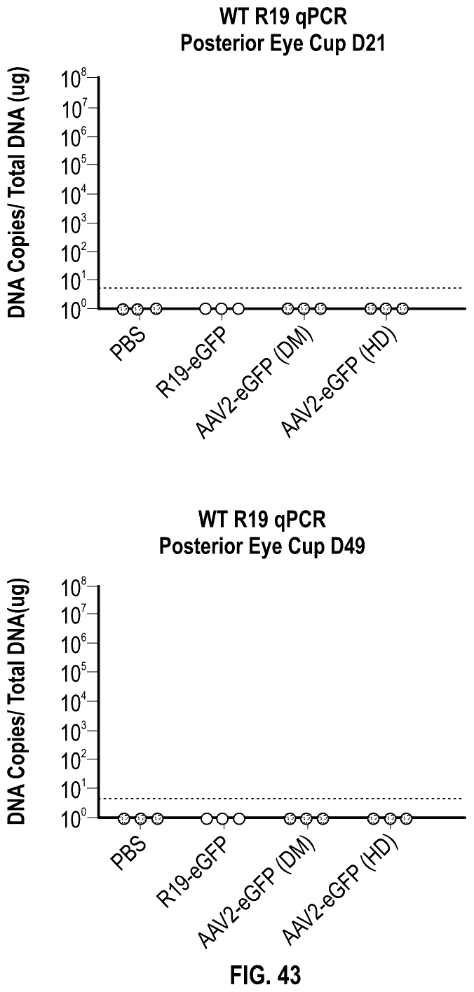

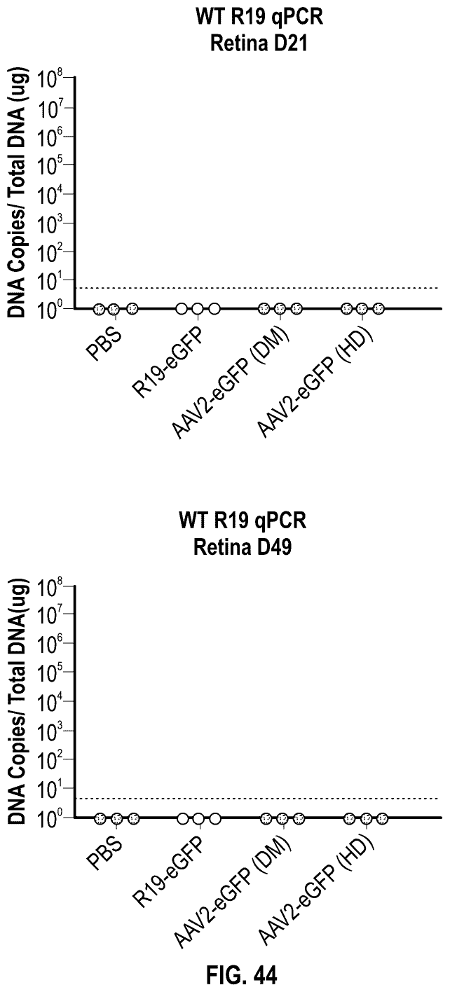

At 21 and 49 days after injection, eye tissue was collected and assessed for the presence of Ring19-eGFP and WT Ring19 genomes using qPCR. Similarly to experiment 1, genomic eGFP was detected in PEC infected by Ring19-eGFP both 21 and 49 days after injection, and continued to persist at greater levels than in the PEC that were infected with dose-matched AAV2-eGFP (FIG. 41). FIG. 42 shows that genomic eGFP likewise persisted in the retina at both 21 and 49 days after injection. This contrasted with WT Ring19, which was not detected in the PEC at 21 or 49 days post injection (FIG. 43) or in the retina at 21 or 49 days post injection (FIG. 44).

Subretinal Injection of Ring19-eGFP Induces eGFP mRNA Expression in the Retina and PEC

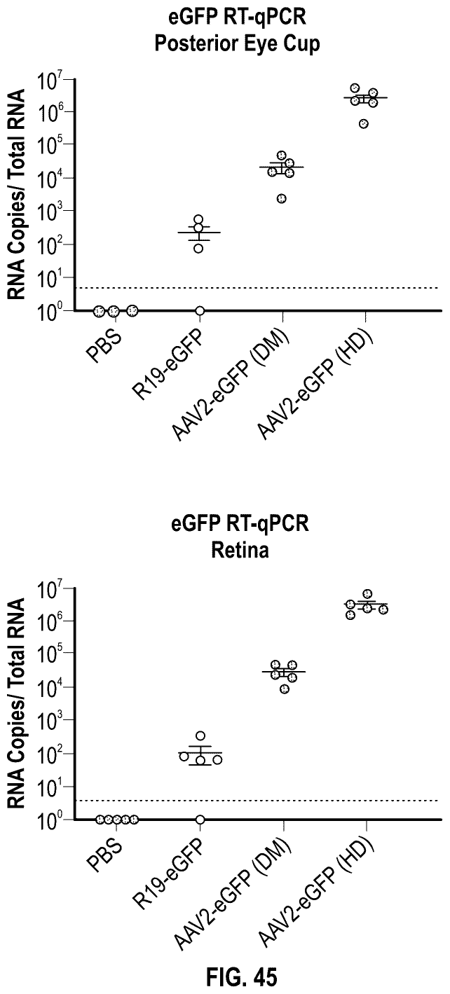

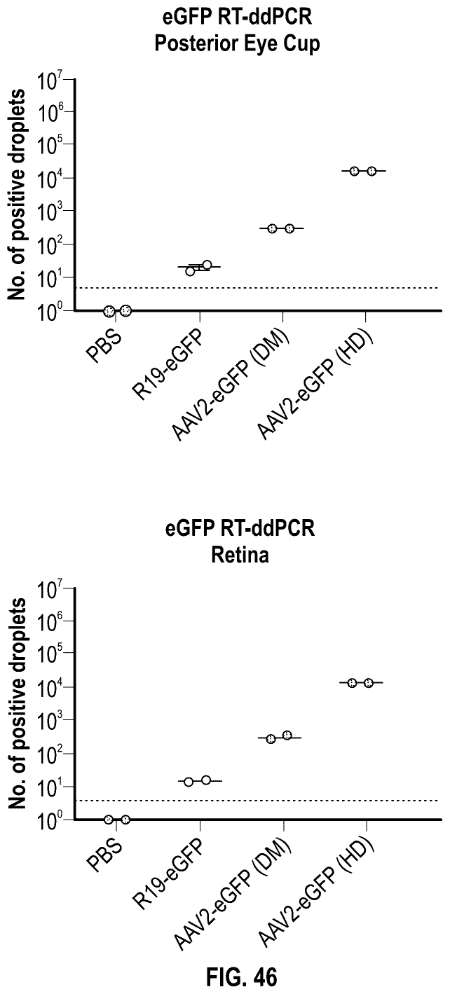

To characterize whether Ring19-eGFP infection can induce eGFP expression in eye tissue, RNA was collected from PEC and retina tissue separately from Experiment 1 (Table AA2) at 21 days after infection (n=5 eyes per group). RT-qPCR targeted to eGFP was then conducted. eGFP mRNA was detected in both the PEC and the retina 21 days after infection by Ring19-eGFP (FIG. 45). eGFP mRNA was also detected in the dose matched and high dose AAV2-eGFP groups, while no eGFP mRNA was detected in the PBS control group (FIG. 45). The presence of eGFP mRNA was confirmed by one-step RT-ddPCR in both the PEC and the retina (FIG. 46), indicating that subretinal injection of Ring19-eGFP successfully transduced eGFP into eye tissue.

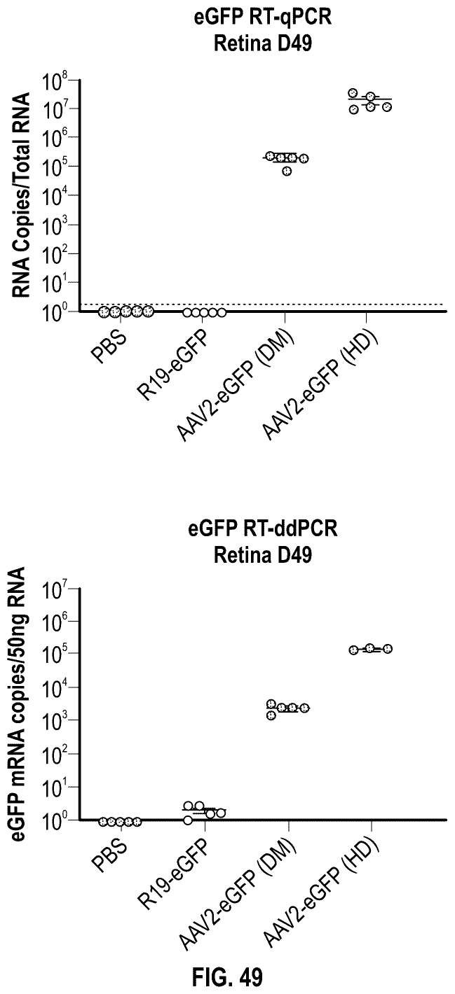

RNA was also collected from Experiment 2 (Table AA3) at 21 days and also extended to a 49 day post injection group. At 21 days after injection, eGFP mRNA was again detected in the PEC and retina in all three experimental groups but not in the PBS treated negative control by both RT-qPCR and RT-ddPCR (FIG. 47). This replicated the data from the previous experiment and showed that eGFP was successfully transduced by Ring19-eGFP. At 49 days post transduction, eGFP mRNA was detected in the PEC by both RT-qPCR and RT-ddPCR (FIG. 48), showing lasting transduction. eGFP mRNA was detected by RT-ddPCR at day 49 in the retina as well but at lower levels than at Day 21 and in the PEC (FIG. 49).

Subretinal Injection of Ring19-eGFP Results in eGFP Protein Expression in the PEC

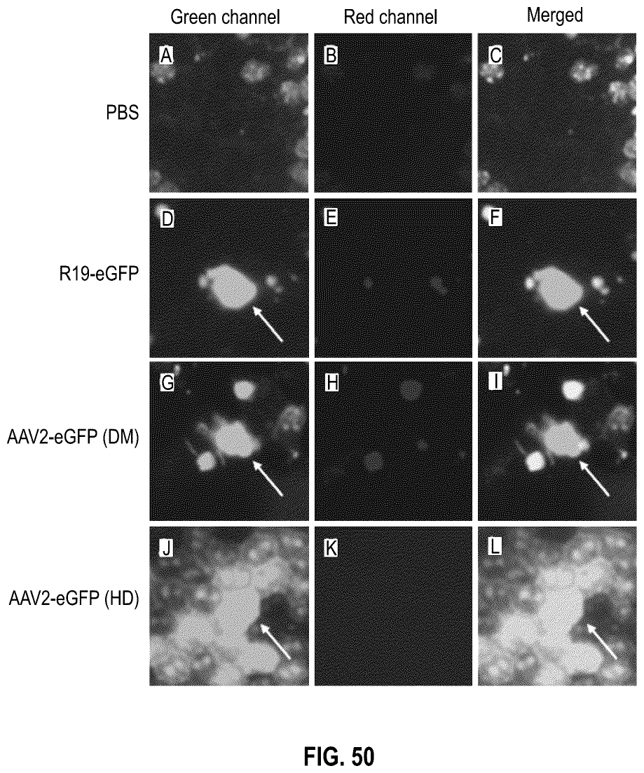

Next, whether functional eGFP protein can be produced after Ring19-eGFP infection was examined. The PEC was collected at day 21 from Experiment 1 for fixation and flatmount imaging (n=2 eyes per group). To determine true eGFP expression, representative sections of the tissue were imaged in both the eGFP and Texas red channels. Overlapping these channels showed that any area where only eGFP signal was detected showed true eGFP expression, while any signal that overlapped between the eGFP and Texas red channels was caused by red blood cell autofluorescence. eGFP positive cells in the Ring-19eGFP group as well as the AAV2-eGFP dose matched and high dose groups were found using this imaging technique (FIG. 50; see arrows). As expected, there were no eGFP positive cells in the PBS treated negative control group (FIG. 50). These data showed that subretinal injection of Ring19-eGFP resulted in expression of functional eGFP protein in the PEC.

### Example 31: Expression of GFP Protein in Human RPE Cells after Transduction with Engineered Ring19 Anellovector

This example describes the transduction of in vitro human retinal pigment epithelial (RPE) cells using engineered Ring19-eGFP anellovector.

### Methods and Materials

### Viral Vector Production

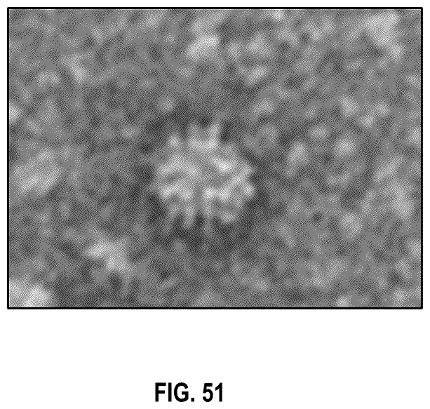

Ring19-eGFP: MOLT-4 cells were transfected with the three in-house designed plasmids (pRTx-2847; pRTx-2848; pRTx-3525; sequences as listed in Tables Z1, Z5, and W1 above) and Ring19-eGFP anellovectors were purified, obtaining a titer of 2.6×10⁹ vg/ml. Virus particle production was verified using TEM imaging (FIG. 51).

AAV2-eGFP was obtained from PackGene Biotech, Inc. with a titer of 1×10¹³ vg/ml.

### Human Retinal Pigment Epithelial Cell Culture

Human RPE cells were obtained from Fujifilm Cellular Dynamics Inc. Cells were thawed and plated in Vitronectin-coated plates and maintained in 91.3% MEM alpha (ThermoFisher), 5% KnockOut SR (ThermoFisher), 1% N-2 Supplement, 55 nM hydrocortisone (Sigma), 250 ug/ml taurine (Sigma), 14 pg/ml triiodo-L-thyronine (T₃) (Sigma), and optional 25 ug/ml gentamicin (ThermoFisher) for 5 days to confluence (FIG. 52).

### Transduction of RPE Cells

RPE cells were transduced on the 5th day of culture using either AAV2-eGFP or Ring19-eGFP at an MOI of approximately 800 (2.6×10⁸ virus particles per 3.2×10⁵ cells). Untreated cells were used as the negative control. The maintenance media was changed on the next day. Cells were cultured for 15 days after transduction with media changes twice per week.

### RPE Fixation and Immunostaining

On the 20th day after thawing, or 15 days after transduction, RPE cells were fixed for immunostaining. RPE were fixed in 4% Paraformaldehyde at room temperature for 20 minutes followed by three washes with 1×DPBS. RPE were then stored in DPBS containing 0.05% sodium Azide at 4C until immunostaining. For immunostaining, fixed RPE cells were permeabilized for 30 minutes at room temperature in a solution of 0.3% Triton X-100, 10% serum, and 5% BSA. Cells were then incubated in a solution containing the primary antibody against ZO-1 (ZO1-1Å12, Alexa Fluor 488, from Invitrogen) used at 1:500 dilution or against GFP (from Abcam) at a dilution of 1:500 in 5% BSA in DPBS overnight at 4 C. Cells were then washed with DPBS three times before application of the goat anti-chicken IgY (H+L) cross-adsorbed secondary antibody, Alexa Fluor Plus 647 (Invitrogen) at a dilution of 1:500 in 5% BSA in DPBS with the addition of Hoechst at a dilution of 1:1000-2000. Cells were incubated in this solution for 2 hours at room temperature, then washed with DPBS three times. 0.05% sodium azide in DPBS was added to each well before imaging.

### Imaging of Live and Fixed RPE Cells

The cells were washed after staining and imaged at room temperature. The imaging was performed using a Zeiss fluorescence microscope (AXIO) and confocal microscope (LSM 900). The images were taken at 20× and 40× magnification. On confocal, the images were taken as a Z-stake. All the images were processed using Fiji (ImageJ) software from the NIH. Different channels were used to image GFP or Alexa Fluor 488 (green), Cy5 (far red), and UV channel in blue for Hoechst (nuclear imaging), along with phase.

### Results

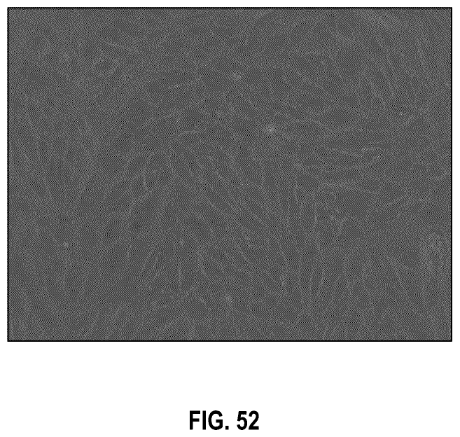

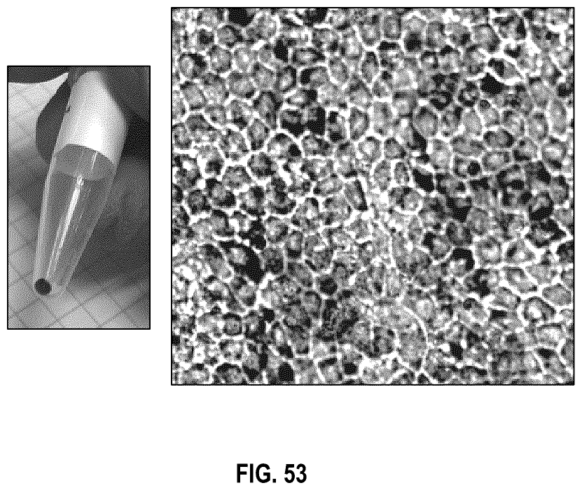

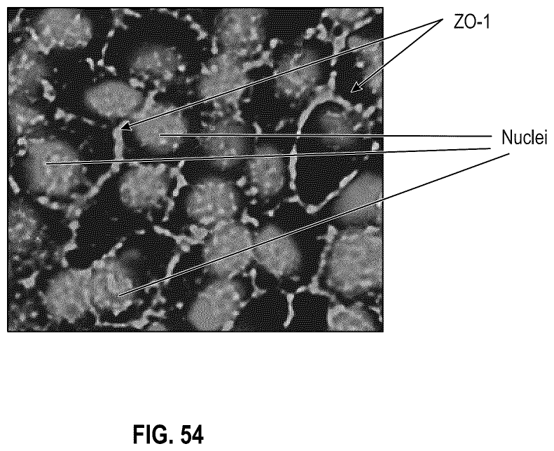

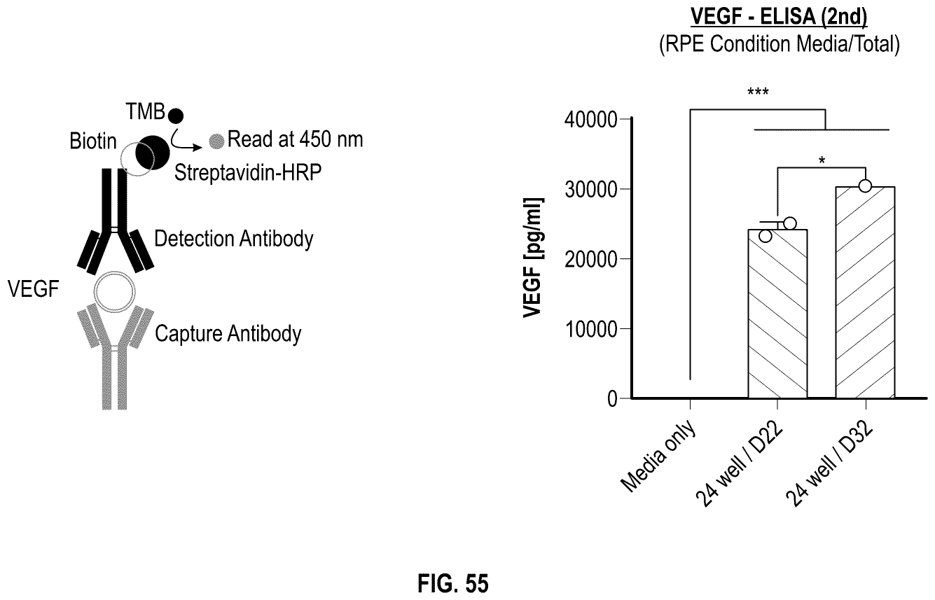

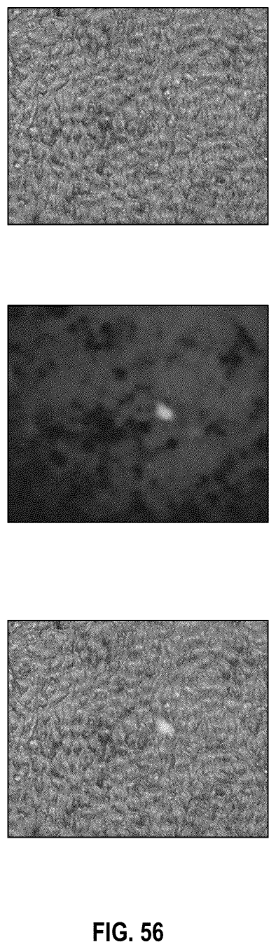

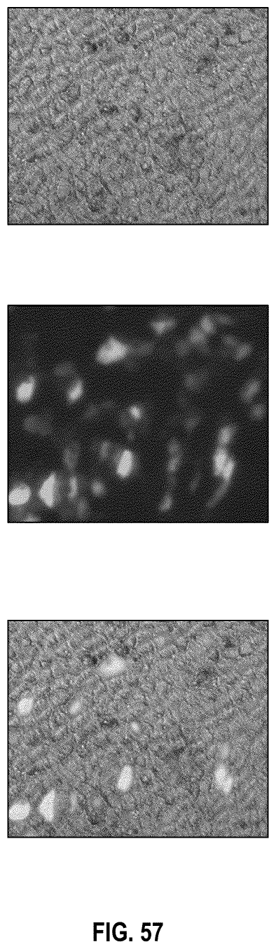

Ring19-eGFP transduction induces GFP protein expression in human RPE cells To determine if Ring19 is able to transduce expression of eGFP in human eye cells, Ring19 virus particles carrying an eGFP expression vector were first produced (Ring19-eGFP). AAV2 virus particles carrying an eGFP expression vector were utilized as a positive control (AAV2-eGFP). Human RPE cells were cultured to confluence as shown in FIG. 51. RPE cells produced melanin granules and formed tight junctions by the 28th day of culture, indicating successful culture of RPEs (FIG. 53 and FIG. 54). Mature RPEs also expressed VEGF protein as expected, as determined by an ELISA assay against VEGF (FIG. 55). Ring19-eGFP or AAV2-eGFP were then used to infect the RPE cell cultures. Live cells imaged 14 days after infection showed eGFP protein expression in cells infected by Ring19-eGFP (FIG. 56) and AAV2-eGFP (FIG. 57), indicating successful transduction of eGFP by Ring19-eGFP.

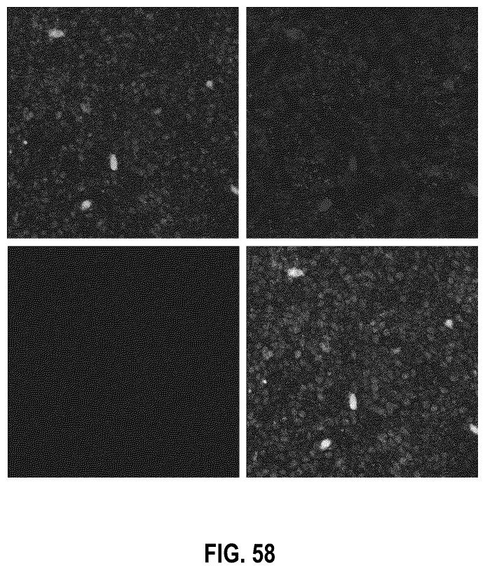

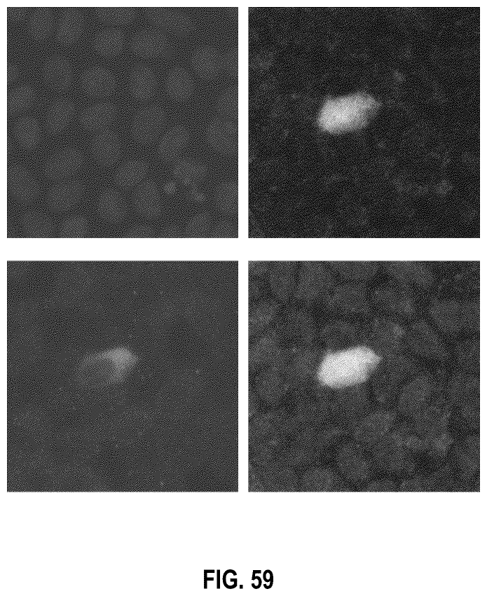

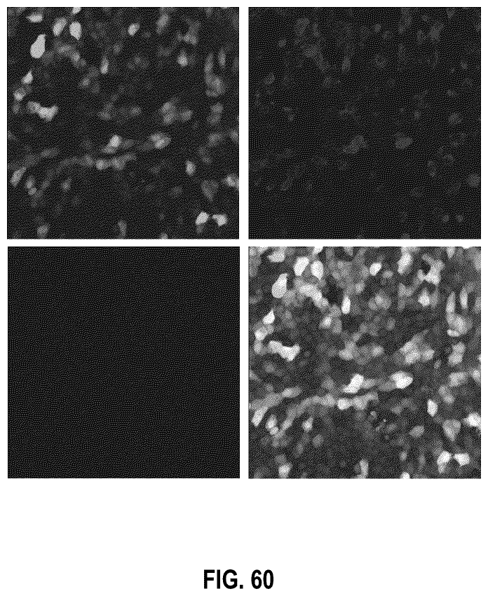

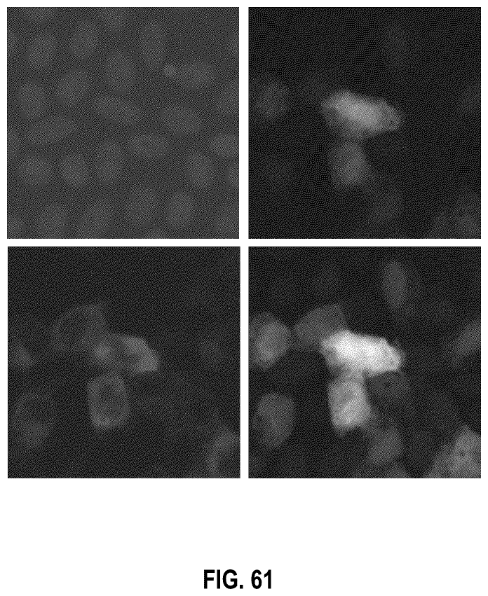

Next, the RPE cells were fixed 15 days after infection for further characterization of induced eGFP protein expression. Fixed cells were immunostained using an antibody against eGFP and imaged for both eGFP protein expression and eGFP protein immunostaining. RPE cells infected with Ring19-eGFP showed eGFP protein expression concurrent with immunostaining for eGFP (FIG. 58 and FIG. 59), as did cells infected with AAV2-eGFP (FIG. 60 and FIG. 61). No eGFP protein expression or eGFP immunostaining signal was detected in untreated cells (FIG. 62). Together, these data demonstrate that a Ring19 anellovector can successfully transduce eGFP to human RPE cells in vitro, demonstrating the utility of anellovectors for ocular delivery of exogenous effectors to human cells.

### Example 32. Transduction of Ocular Tissue with Varying Doses of Ring19-eGFP

This example describes the administration of different dosage levels of Ring19-eGFP anellovector to ocular tissue and the corresponding dose response. Ring19-eGFP was administered at two different doses, here termed high dose (HD) and low dose (LD).

### Preparation of Viral Particles

AAV2-fCMV-eGFP was prepared by Packgene Biotech Inc. and diluted in sterile 1×PBS. Ring19-fCMV-eGFP was prepared as described in Example 30.

### Subretinal Injection of Various Dose Levels of Viral Particles and Analysis

Ring19-eGFP and AAV2-eGFP particles were subretinally injected into mice at the dose described in Table AA4 below, according to the method described in Example 30.

All analytical methods were performed according to the methods described in Example 30.

TABLE AA4
Experimental design

| | | | Dose | | | | |
|---|---|---|---|---|---|---|---|
| | | | Vector | Dose/eye | | N | Terminal |
| Group | Strain | Treatment Day 0 | (vg/ml) | (vg) | Route | (eyes) | Day |
| 1 | C57BL/6J | PBS | 0 | 0 | SR | 12 | 21 |
| | | | | | (1 ul) | | |
| 2 | C57BL/6J | R19-fCMV-eGFP | 1.2e+10 | 1.2e+7 | SR | 12 | 21 |
| | | (LD) | | | (1 ul) | | |
| 3 | C57BL/6J | R19-fCMV-eGFP | 1.2e+11 | 1.2e+8 | SR | 12 | 21 |
| | | (HD) | | | (1 ul) | | |
| 4 | C57BL/6J | AAV2-fCMV-eGFP | 1.2e+10 | 1.2e+7 | SR | 12 | 21 |
| | | (LD) | | | (1 ul) | | |
| 5 | C57BL/6J | AAV2-fCMV-eGFP | 1.2e+11 | 1.2e+8 | SR | 12 | 21 |
| | | (HD) | | | (1 ul) | | |

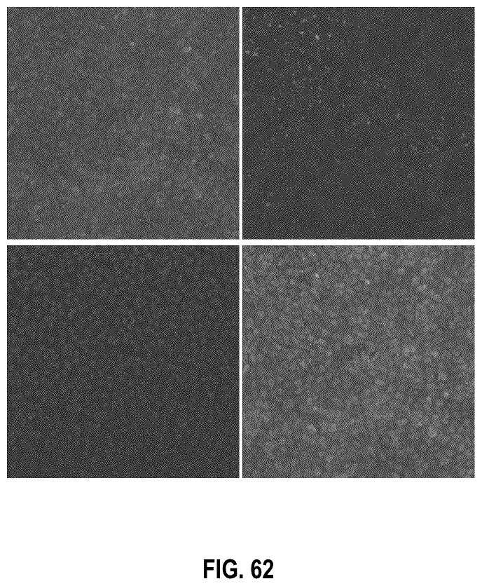

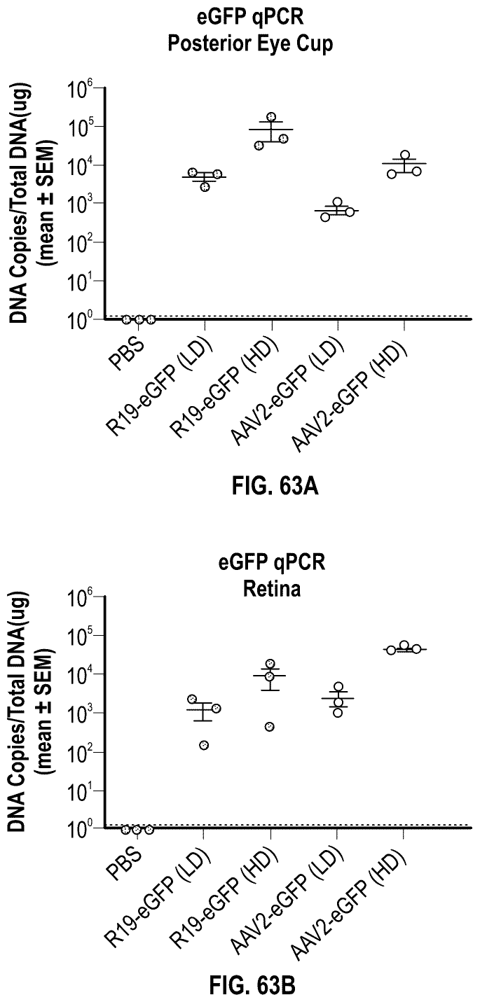

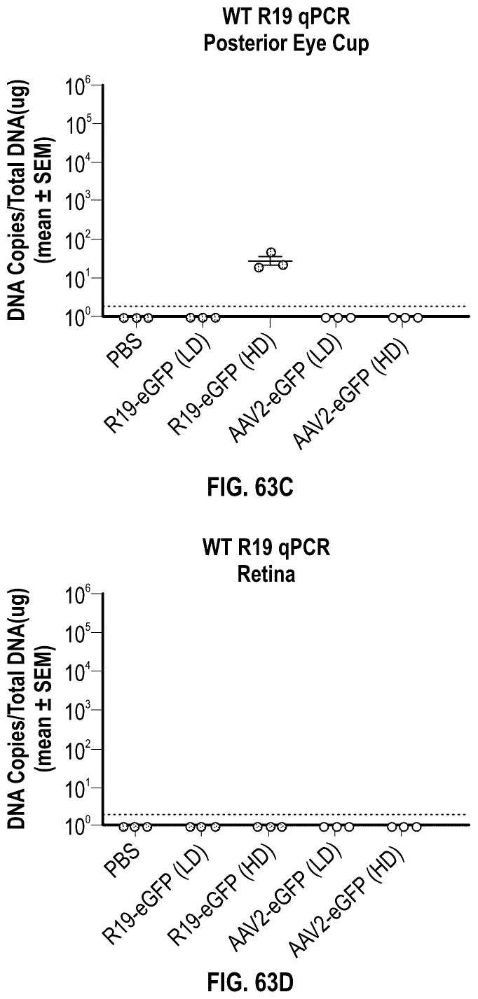

21 days after injection, DNA from the posterior eye cup (PEC) and retina were collected and processed separately. eGFP genomes were detected by qPCR in the PEC and the retina (FIGS. 63A and 63B). The level of eGFP genomic DNA increased in a doses-dependent manner in the PEC and retina for both Ring19-eGFP and AAV2-eGFP. Furthermore, the eGFP genomes detected were significantly greater in the PEC infected with Ring19-eGFP compared to PEC infected with dose-matched AAV2-eGFP at both the low dose and high dose (FIG. 63A). A very low level of WT Ring19 genome was detected in the PEC infected with Ring19-eGFP (HD) (FIG. 63C) but no WT Ring19 genome was detected in the Ring19-eGFP (LD) or in the retina (FIG. 63D).

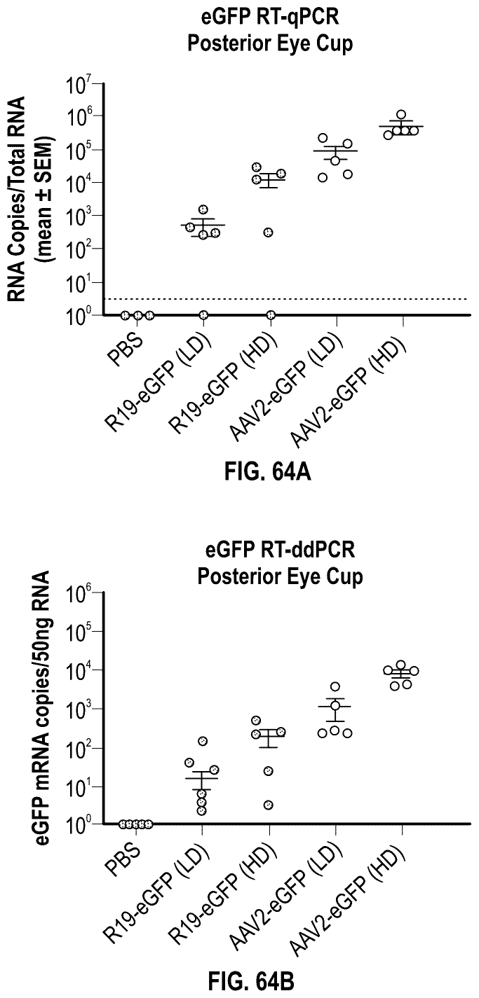

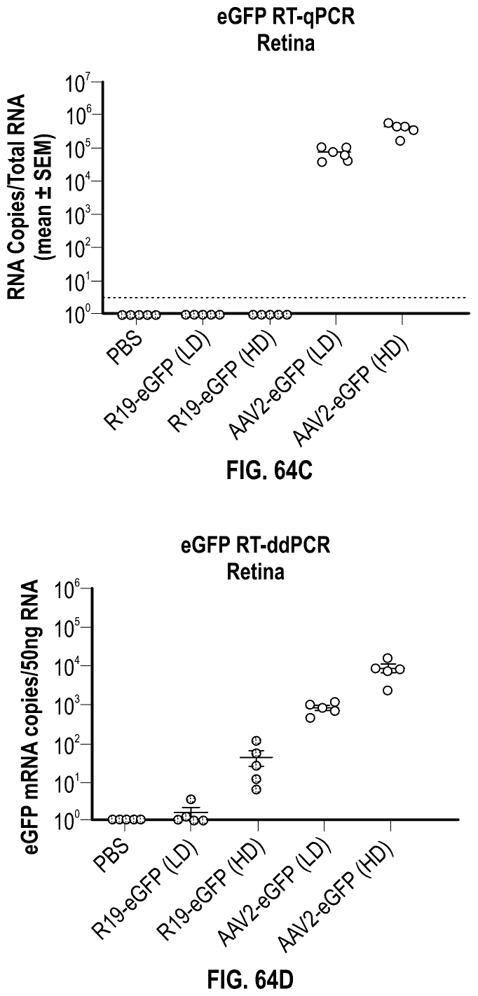

To characterize whether Ring19-eGFP infection can induce eGFP expression in eye tissue, RNA was collected from PEC and retina tissue separately at 21 days after transduction (n=5 eyes per group). RT-qPCR targeted to eGFP was then conducted. eGFP mRNA was detected in a dose-dependent manner in the PEC 21 days after transduction by LD and HD Ring19-eGFP (FIG. 64A). eGFP mRNA was also detected in the dose matched AAV2-eGFP groups, while no eGFP mRNA was detected in the PBS control group (FIG. 64A). The presence of eGFP mRNA was confirmed by one-step RT-ddPCR in the PEC (FIG. 64B). eGFP mRNA was below the limit of detection in the retina transduced with LD and HD Ring19-eGFP (FIG. 64C) but eGFP mRNA was detected by one-step RT-ddPCR in the retina transduced with HD Ring19-eGFP (FIG. 64D).

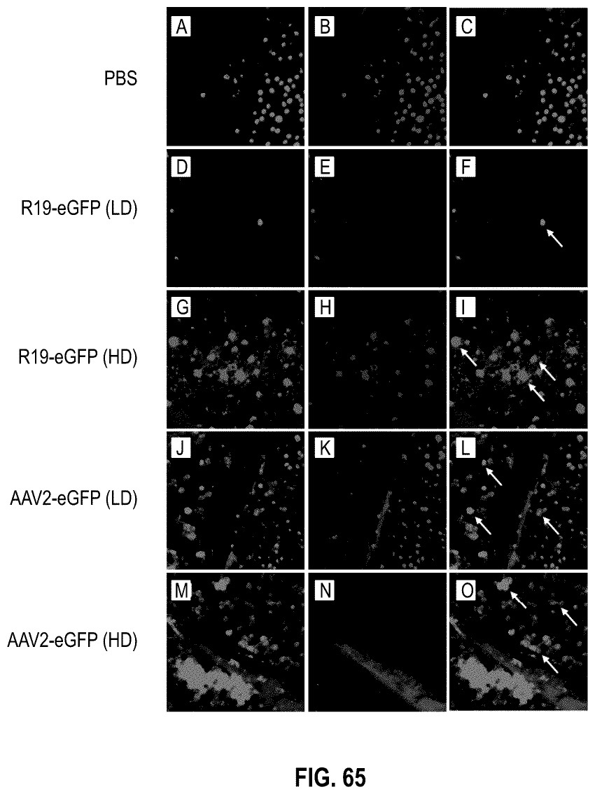

Next, whether functional eGFP protein can be produced after Ring19-eGFP transduction was examined. The PEC was collected at day 21 for fixation and flatmount imaging (n=3 eyes per group). To determine true eGFP expression, representative sections of the tissue were imaged in both the eGFP and Texas red channels. Overlapping these channels showed that any area where only eGFP signal was detected showed true eGFP expression, while any signal that overlapped between the eGFP and Texas red channels was caused by red blood cell autofluorescence. eGFP positive cells in the LD and HD Ring19-eGFP groups as well as in the LD and HD AAV2-eGFP dose matched groups were found using this imaging technique (FIG. 65; see arrows). Moreover, greater number of eGFP positive cells were detected in the HD Ring19-eGFP group compared to the LD Ring-19-eGFP group. As expected, there were no eGFP positive cells in the PBS treated negative control group (FIG. 65). These data showed that subretinal injection of Ring19-eGFP resulted in expression of functional eGFP protein in the PEC.

Together, these data show that more eGFP genomes, more eGFP transcripts, and more eGFP-positive cells could be delivered and expressed in eye tissue with higher doses of the Ring19 anellovector and in a dose-dependent manner.

### Example 33. A Cre Recombinase Based Three-Plasmid System for Anelloviridae Family Vector Particle Production

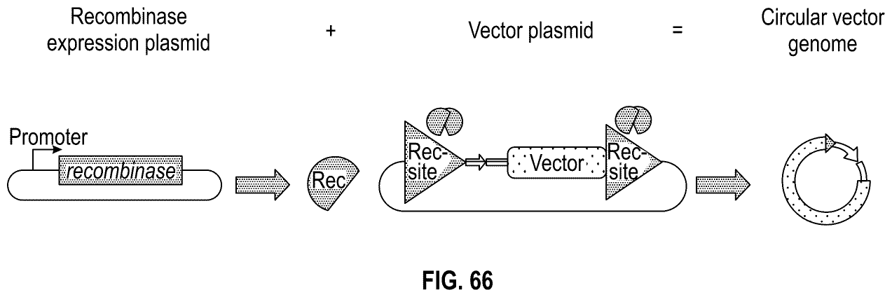

In this example, a site specific recombinase, such as Cre, is used to control the site of recombination of a genetic element construct by specific placement of recombinase recognition sites (e.g., loxP sites) flanking the sequence of the genetic element. An exemplary genetic element construct includes non-coding regions of an Anelloviridae family viral genome, which contain the elements required for DNA replication and packaging, as well as a transgene cassette (e.g., including a promoter, transgene, and terminator elements). By flanking this cassette with recombinase recognition sites, the recombination event can be used to produce double-stranded DNA minicircles consisting of the excised genetic element sequence (FIG. 66). The minicircle can then be converted to single-stranded circular DNA, which can undergo rolling circle replication mediated by Anelloviridae family proteins and/or be packaged into particles as described herein.

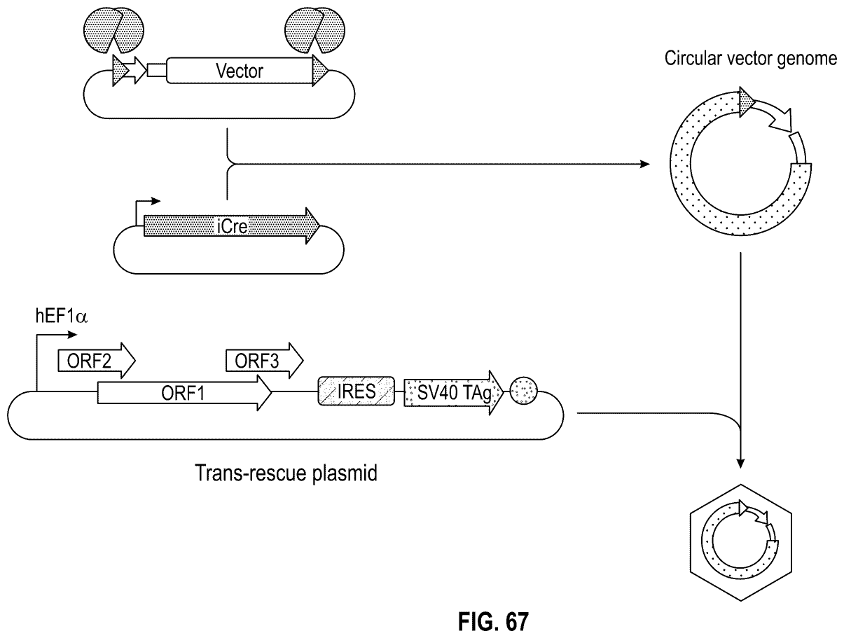

In this example, producer cells are transfected in a 3-plasmid system comprising: (1) a first plasmid comprising recombinase recognition sites (e.g. loxp) flanking an expression cassette with the non-coding region (NCR) of an Anelloviridae family virus followed by a promoter, transgene, and terminator element, (2) an expression plasmid that provides the recombinase (e.g. Cre), and (3) a self-replicating rescue (SRR) plasmid or another rescue plasmid variant that contains Anelloviridae family viral proteins to drive replication and packaging of the circular genetic elements (FIG. 67). Experiments demonstrating the use of the three-plasmid system using Cre recombinase and loxp recognition sites specifically are provided below.

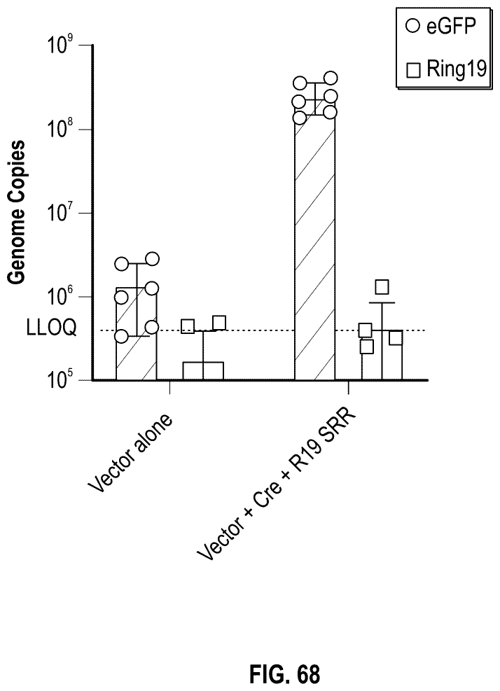

In one experiment, two experimental groups were tested. In one, only the vector plasmid was transfected into cells. In the second, all three plasmids (vector plasmid, Cre recombinase expression plasmid, and SRR plasmid) were transfected into cells together. qPCR was performed on particles produced to quantify genome copies of WT Ring19 versus eGFP. The three-plasmid transfection produced more Ring19-eGFP genome copies than the vector alone transfection, and the WT Ring19 genome copies remained at below the lower limit of quantification (LLOQ) (FIG. 68). This demonstrates that the three-plasmid recombinase system is effective at producing Ring19-eGFP particles with no detectable WT Ring19 contamination.

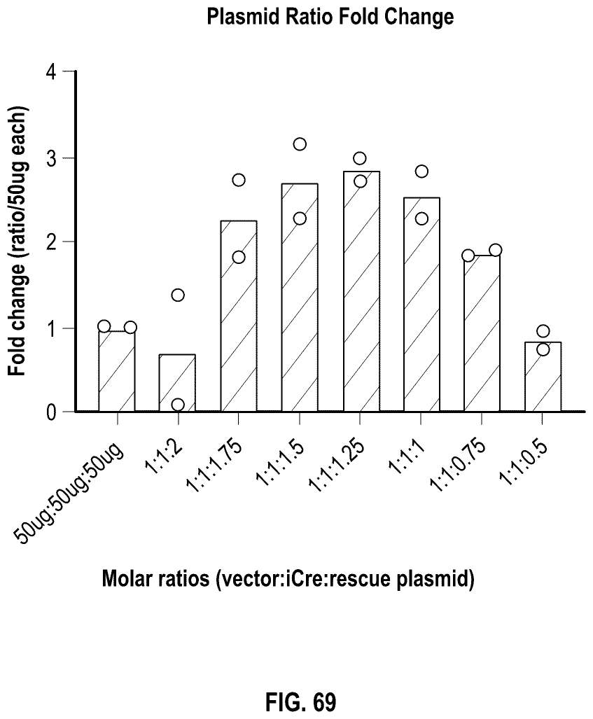

To further characterize the three-plasmid system, cells were transfected with the three plasmids in varying molar ratios. The molar ratio of the vector plasmid and the Cre recombinase plasmid were kept constant with varying amounts of the SRR plasmid. The ratios tested included 1:1:2, 1:1:1.75, 1:1:1.5, 1:1:1.25, 1:1:1, 1:1:0.75, 1:1:0.5 (vector:cre:SRR). In this experiment, the 1:1:1.25 ratio produced the highest quantity of particles with Ring19-eGFP genomes (FIG. 69).

### Example 34. A Bxb1 Recombinase Based Three-Plasmid System for Anellovector Viral Particle Production

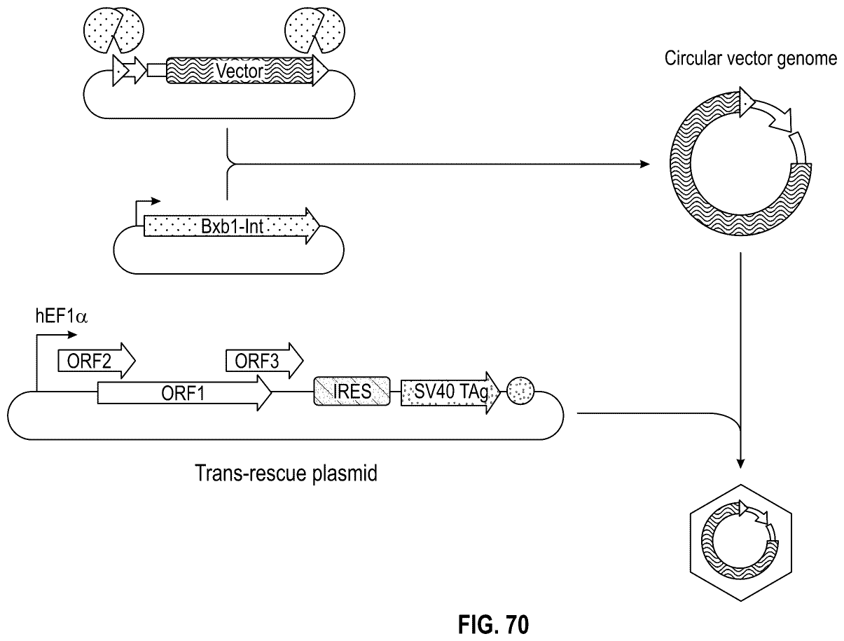

In this example, an exemplary three-plasmid recombinase system utilizing Bxb1 is used to produce minicircles comprising Anelloviridae family vector genetic element sequences. Bxb1 provides, in some instances, reduced toxicity in human cells, high recombination efficiency in human cells, unidirectional recombinase function, and limited recognition sequences in the human genome. In this system, producer cells are transfected with 3 plasmids: (1) a first plasmid comprising recombinase recognition sites (attP and attB sites) flanking an expression cassette comprising the non-coding region (NCR) of an Anelloviridae family virus, a promoter, a transgene, and a terminator element, (2) an expression plasmid that encodes Bxb1 recombinase, and (3) a self-replicating rescue (SRR) plasmid that encodes Anelloviridae family viral proteins to drive replication and packaging of the circular genomic elements (FIG. 70).

To demonstrate the three-plasmid system using Bxb1, the following plasmids were prepared: a vector plasmid with Bxb1 recognition sites flanking a UBC driven eGFP expression cassette (pBox), a Bxb1 expression plasmid, and an SRR with Ring19 ORF proteins. Two variants of the pBox plasmid were made, each containing a single point mutation between the Cap site and 5′ conserved UTR in the Ring19 NCR to remove an Esp31 restriction enzyme site for cloning purposes. pBoxl remained unmutated. pBox2 contains an A to T mutation at nucleotide 2839 of Table N25 or nucleotide 200 of Table N26.1, and a G to C mutation at nucleotide 2838 of Table N25 or nucleotide 199 of Table N26.1 of the Ring19 genome sequence.

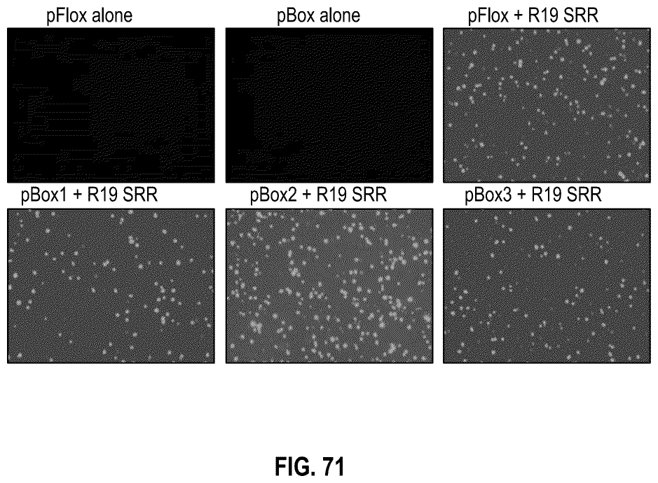

In one experiment, recombination and transgene expression efficiency of the Bxb1 and Cre recombinase-based systems were tested. MOLT-4 cells were transfected with either: the pFlox vector with CMV as the eGFP transgene promoter or the pBox vectors (pBoxl, pBox2, and pBox3) with UbC as the eGFP transgene promoter, alone or together with a Ring19 self-replicating rescue plasmid (SRR) and a Cre or Bxb1-expressing plasmid. Cells were imaged for eGFP protein expression four days after transfection. Cells transfected with pFlox and the Ring19 SRR, and any of the pBox variants with the Ring19 SRR, exhibited expression of eGFP, but not when pFlox or pBox alone were transfected (FIG. 71). These results demonstrated that the recombinase system, using either Cre or Bxb1 recombinase, resulted in successful expression of transgene in producer cells.

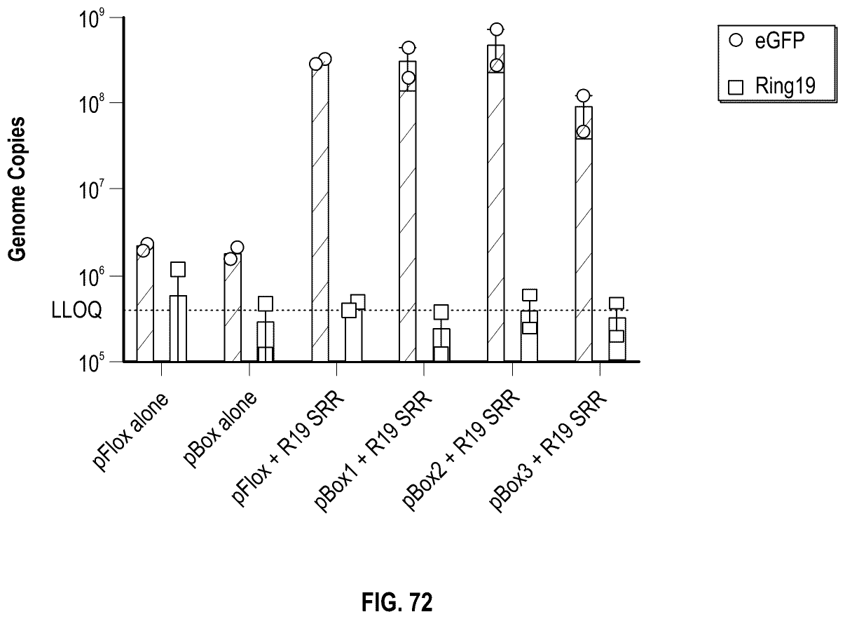

Transfected MOLT-4 cells were harvested, lysed, and viral particles were purified through an iodixanol linear gradient. Following DNAse I digestion and proteinase K digestion, genome copies of eGFP or WT Ring19 was quantified by qPCR. Based on two replicates, the four groups that included transfection of an SRR plasmid and a recombinase-expressing plasmid showed higher numbers of eGFP genomes than the pFlox or pBox alone groups, and the WT Ring19 levels were at or below the LLOQ (FIG. 72).

Together, these data show that the recombinase systems described herein can effectively produce Ring19-eGFP over WT Ring19 genetic elements. These genetic elements were also DNase-protected, showing successful packaging into particles.

Without wishing to be bound by theory, it is contemplated that other site-specific recombinases having specific recognition sites can be utilized in the recombinase systems described herein. Non-limiting examples of such site-specific recombinases and their corresponding recognition sites are listed in Table V1 herein.

### Example 35. Different Promoters can Drive Transgene Expression in the Cre and Bxb1 Recombinase Systems

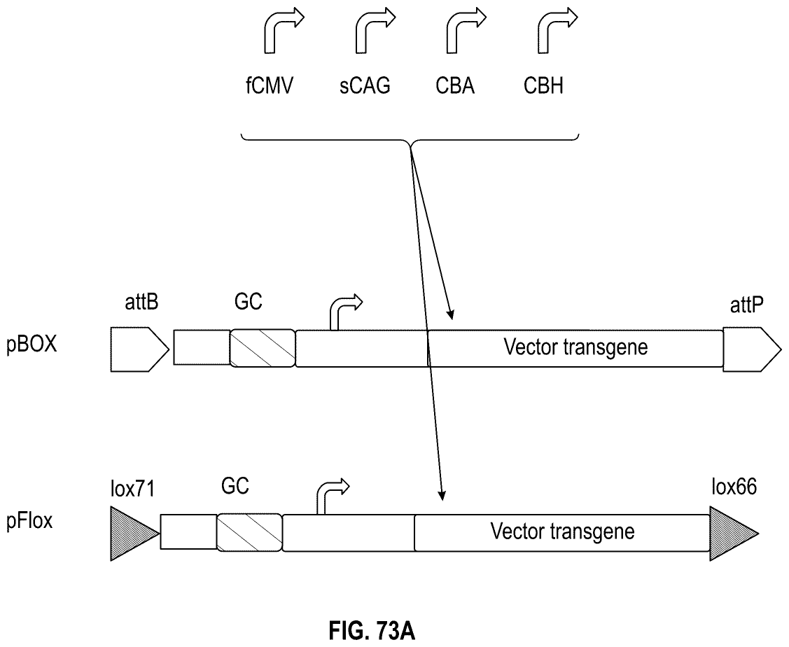

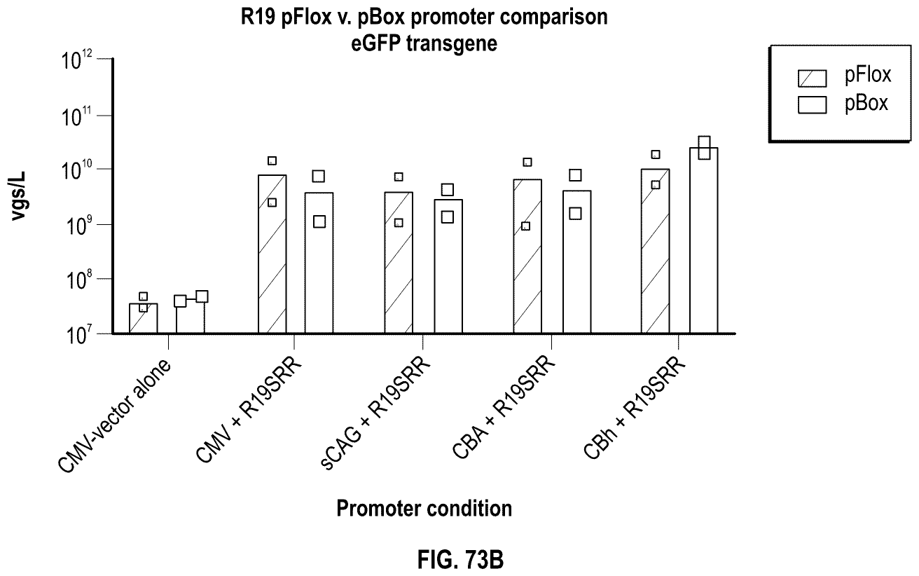

In this example, the use of several different promoters to drive expression of the vector transgene in both the Cre and Bxb1 recombinase three-plasmid systems was tested. Universal promoters fCMV, sCAG, CBA, and CBH were inserted into the vector transgene while keeping the transgene itself, eGFP, constant (FIG. 73A). MOLT-4 cells were transfected with either the Cre or the Bxb1 recombinase three-plasmid systems with a Ring19 self-replicating rescue (SRR) plasmid and the resulting virus particles were isolated after 4 days, lysed, and purified through an iodixaol linear gradient as described herein. A DNase-protection assay was performed on the isolated viral material, and qPCR was used to detect the number of viral Ring19-eGFP genomes per liter of cell culture.

Based on two replicates, payload-matched Cre and Bxb1 three-plasmid systems yielded comparable quantities of Ring19-eGFP viral genomes when using the same transgene promoter (FIG. 73B). Both the Cre and Bxb1 systems also performed comparably across different transgene promoters (FIG. 73B). These data show that the Cre and Bxb1 three-plasmid systems yielded similar amounts of vector genomes, and that different promoters can be used within both systems. These data also indicate that tissue-specific promoters can be utilized to tailor transgene expression to specific target tissues of interest.

### Example 36. Enhancing Vector Production Using Alternative Origins of Replication

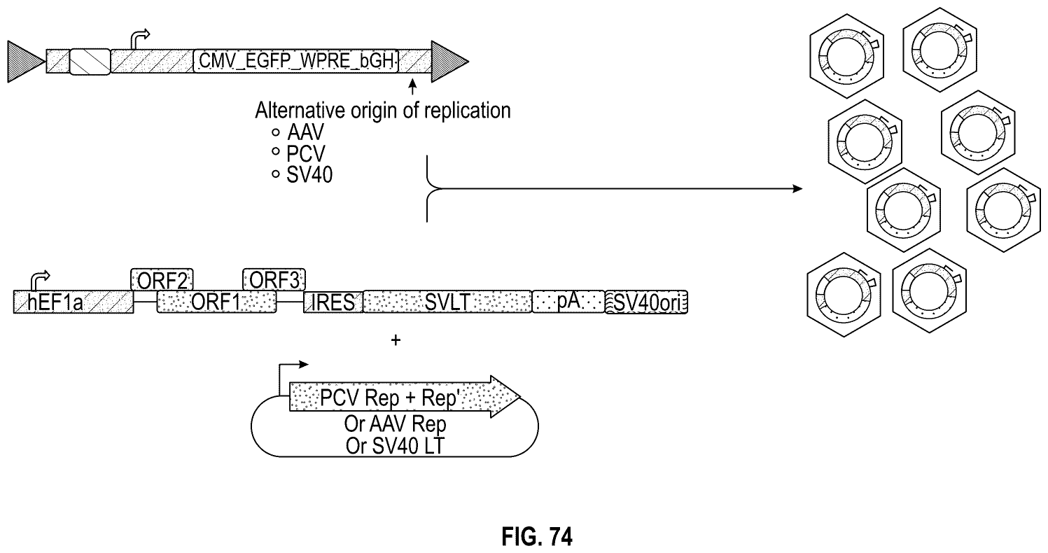

This example describes insertion of different origins of replication, such as an origin of replication derived from AAV, PCV, or SV40 viruses, into Anelloviridae family vector genetic element plasmids, e.g., to alter or enhance production of Anelloviridae family vector particles. FIG. 74 depicts the use of an exemplary plasmid containing a transgene cassette with an alternative origin of replication, flanked by recombinase sites, in combination with an SRR plasmid and, in this example, a plasmid encoding the replication initiator proteins for the selected origin of replication. When co-transfected into a cell expressing a recombinase (either by transfecting a plasmid encoding a recombinase or stably producing a recombinase), these plasmids can be used to produce Anelloviridae family vector particles. The origin of replication can be selected and/or optimized for maximal production of particles.

### Example 37. A Recombinase Based Two-Plasmid System for Anelloviridae Family Vector Particle Production

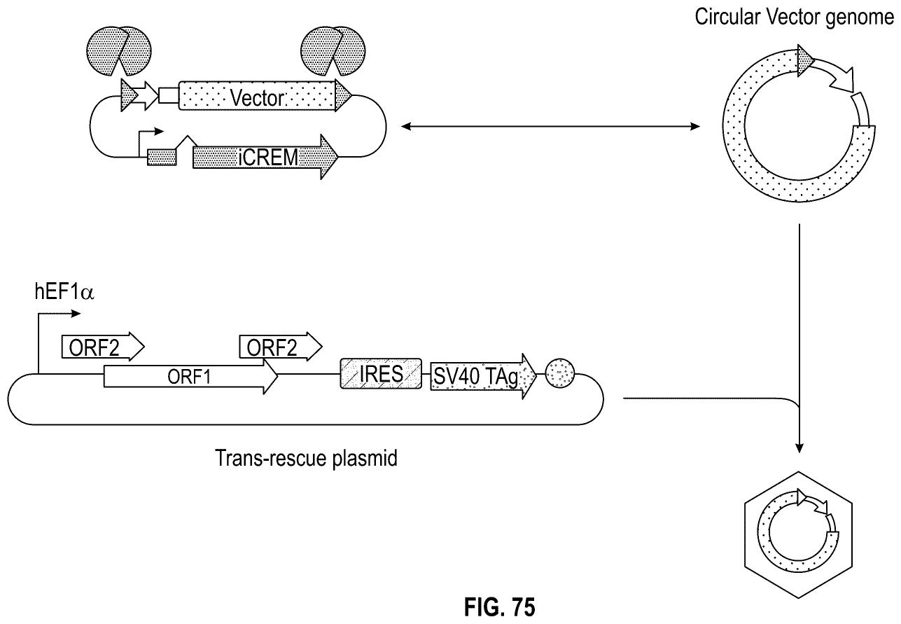

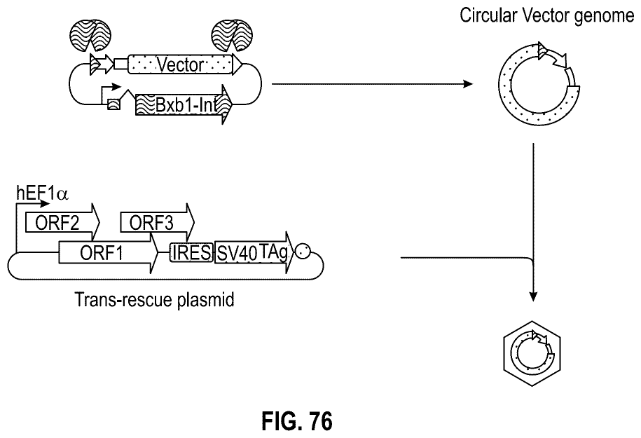

This example describes an exemplary Cre recombinase-based system that utilizes two plasmids instead of three. In this system, the cassette encoding the recombinase is incorporated onto the vector plasmid, resulting in a 2-plasmid system comprising the vector plasmid, which comprises both the genetic element sequence and the recombinase expression cassette, and an SRR plasmid as described herein (FIG. 75). This method ensures that the ratio of recombinase DNA sequence to genetic element sequence is 1:1. Bxb1 recombinase can also be utilized in a similar two-plasmid system (FIG. 76), as can other site-specific recombinases with specific associated recognition sites (e.g., as described herein).

In on example, MOLT-4 cells are transfected with the two plasmids of the system, and Anelloviridae family vector particles carrying genetic elements are isolated and purified as described previously herein. A DNase-protection assay can be used to quantify the production of packaged particles, and qPCR for vector specific DNA and WT Ring DNA can be used to determine the amount of vectorized particles produced relative to WT viral particles. It is contemplated that an increase in the vector specific Ring genomes over WT Ring genomes would indicate that the two-plasmid system preferentially produces packaged vector genetic elements.

### Example 38. Isolation of cssDNA Using Nicking Enzymes and Exonuclease Digest

The genomes of Anelloviridae family viruses consist of circular single stranded DNA (cssDNA). This example describes the use of nicking enzymes and digestion with an exonuclease to produce cssDNA from a double-stranded DNA molecule, such as a minicircle as described herein. Minicircles can also be produced via the recombinase-based methods and constructs described herein (e.g., via site-specific recombination of a vector plasmid comprising a genetic element sequence flanked by recombinase recognition sites).

Once a minicircle has been produced, it can be converted to cssDNA. In one example, the minicircle may be first nicked on one strand using a nicking enzyme such as the Nt.BsmAI enzyme. The nicked strand is then digested by an exonuclease (e.g. T7 exonuclease), leaving the other strand intact. Gel electrophoresis may be used to analyze the untreated, nicked, and nicked and exonuclease treated DNA. A southern blot may also be performed on a gel with probes to detect the antisense and the sense strand of the DNA. After completion of both nicking and exonuclease digestion, only the antisense strand of the DNA is detected. This demonstrates that the combination of nicking and exonuclease digestion successfully isolates the antisense strand of the starting DNA as a circular single stranded DNA molecule.

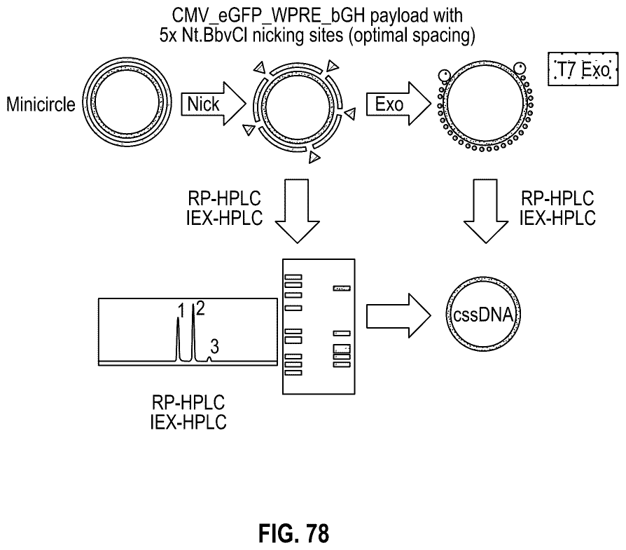

The minicircle may be designed such that it comprises one or more nicking sites (FIG. 78). In an embodiment, the minicircle may comprise 1, 2, 3, 4, 5, or more nicking sites that are optimally spaced. The minicircle may be incubated with a nicking enzyme (e.g., Nt.BbvCI (New England Biolabs)), which nicks the sense strand of the plasmid at the 1, 2, 3, 4, 5, or more nicking sites. An exonuclease, such as T7 exonuclease, is used to digest the nicked strand, leaving the antisense strand intact as a cssDNA (FIG. 78). The cssDNA is then physically separated from the debris of the nicked strand and can be isolated and purified as shown in FIG. 78.

### Example 39. Isolation of cssDNA from a Ring19-eGFP Minicircle Using Nicking Enzymes and Exonuclease Digest

This example demonstrates the isolation of cssDNA from a double stranded minicircle by selective nicking of the positive-sense strand of the minicircle, digesting the nicked strand with an exonuclease, and isolating the negative strand cssDNA by phenol:choloroform extraction and ethanol precipitation.

Selective nicking of positive-sense strand

Minicircle pRTx-3929_MC as shown in Table Y4 was incubated with at least 1 U of the nicking enzyme Nt·BbvCl per μg of DNA as shown in Table 38A1.

TABLE 38A1

| Component | Volume (μl) |
|---|---|
| pRTx-3929_MC (5000 μg) | 1498.8 |
| 10x rCutSmart ™ Buffer (NE Biolabs) | 500 |
| Nt.BbvCl (10 U/μl) | 550 |
| Water | 451.2 |
| Total | 3000 |

The above mixture was incubated at 37° C. for 2.5 hours.

### Exonuclease Digest of Nicked Strand

The nicking reaction mixture was then directly incubated with T7 exonuclease (at least 1 μl per μg of DNA) as shown in Table 38A2.

TABLE 38A2

| Component | Volume (μl) |
|---|---|
| Nicking reaction mixture | 1500 |
| T7 exonuclease | 2500 |
| 10x rCutSmart ™ Buffer (NE Biolabs) | 1250 |
| Water | 8750 |
| Total | 14000 |

The above mixture was incubated at 25° C. (about room temperature) for 30 minutes. The reaction was stopped by adding EDTA to 11 mM. The concentration of the DNA was 178.6 ng/μl.

Isolation of the Negative-Strand cssDNA

One volume of a phenol:chloroform solution was added to the exonuclease-digested mixture and vortexed vigorously for 1 minute. The tube was centrifuged for 5 minutes and the aqueous phase was aspirated from the top of the solution and transferred to a new clean tube.

1/10^{th} volume of 3M sodium acetate solution was added to the aspirated nucleic acid sample, followed by 1 volume of 99% isopropanol. The mixture was centrifuged for 30 minutes at 4° C. at 12,000×g. The supernatant was aspirated or decanted, retaining the pellet. The pellet was rinsed with cold 70% ethanol and centrifuged for 5-15 minutes at 4° C. at 12,000×g. The supernatant was aspirated or decanted, retaining the pellet. The pellet was allowed to airdry and then dissolved in water. The concentration of the cssDNA was 1619.6 ng/μl.

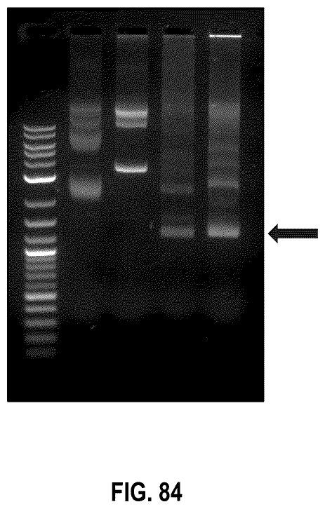

FIG. 84 shows a gel with a 1 kb plus DNA ladder (lane 1), untreated DNA (400 ng) (lane 2), nicked DNA (400 ng) (lane 3), nicked and exonuclease-treated unpurified DNA (1 μg) (lane 4), and nicked and exonuclease-treated purified DNA (1 μg) (lane 5). As shown in lane 5, the purified cssDNA showed the expected banding pattern (shown with an arrow).

### Example 40. Isolation of cssDNA from a Ring2-eGFP Minicircle Using Nicking Enzymes and Exonuclease Digest

This example demonstrates the isolation of cssDNA from a double stranded minicircle by selective nicking of the positive-sense strand of the minicircle, digesting the nicked strand with an exonuclease, and isolating the negative strand cssDNA by phenol:choloroform extraction and ethanol precipitation.

### Selective Nicking of Positive-Sense Strand

Minicircle pRTx-3928_MC as shown in Table Y2 was incubated with at least 1 U of the nicking enzyme Nt·BbvCl per μg of DNA as shown in Table 38B3.

TABLE 38B3

| Component | Volume (μl) |
|---|---|
| pRTx-3928_MC (12000 μg) | 6141 |
| 10x rCutSmart ™ Buffer (NE Biolabs) | 850 |
| Nt.BbvCl (10 U/μl) | 1320 |
| Water | 189 |
| Total | 8500 |

The above mixture was incubated at 37° C. for 2.5 hours.

### Exonuclease Digest of Nicked Strand

The nicking reaction mixture was then directly incubated with T7 exonuclease (at least 1 μl per μg of DNA) as shown in Table 38B4.

TABLE 38B4

| Component | Volume (μl) |
|---|---|
| Nicking reaction mixture | 2833.3 |
| T7 exonuclease | 4000 |
| 10x rCutSmart ™ Buffer (NE Biolabs) | 1716.7 |
| Water | 11450 |
| Total | 20000 |

The above mixture was incubated at 25° C. (about room temperature) for 30 minutes. The reaction was stopped by adding EDTA to 11 mM. The concentration of the DNA was 50 ng/μl.

Isolation of the Negative-Strand cssDNA

One volume of a phenol:chloroform solution was added to the exonuclease-digested mixture and vortexed vigorously for 1 minute. The tube was centrifuged for 5 minutes and the aqueous phase was aspirated from the top of the solution and transferred to a new clean tube.

1/10^{th} volume of 3M sodium acetate solution was added to the aspirated nucleic acid sample, followed by 1 volume of 99% isopropanol. The mixture was centrifuged for 30 minutes at 4° C. at 12,000×g. The supernatant was aspirated or decanted, retaining the pellet. The pellet was rinsed with cold 70% ethanol and centrifuged for 5-15 minutes at 4° C. at 12,000×g. The supernatant was aspirated or decanted, retaining the pellet. The pellet was allowed to airdry and then dissolved in water. The concentration of the cssDNA was 1638.6 ng/μl.

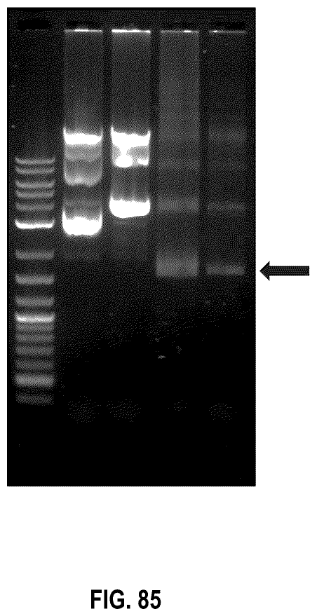

FIG. 85 shows a gel with a 1 kb plus DNA ladder (lane 1), untreated DNA (lane 2), nicked DNA (lane 3), nicked and exonuclease-treated unpurified DNA (lane 4), and nicked and exonuclease-treated purified DNA (lane 5), all loaded with 500 ng of DNA. As shown in lane 5, the purified cssDNA showed the expected banding pattern (shown with an arrow).

### Example 41. Production of Minicircles in Bacterial Cells Using a Recombinase System

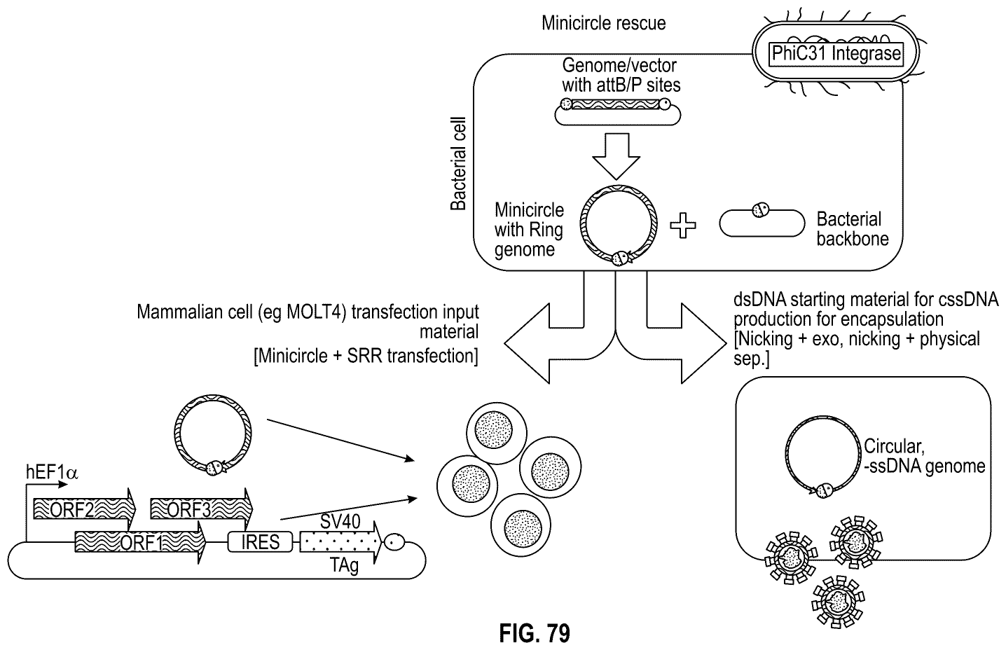

This example describes the generation of double-stranded DNA minicircles comprising the genetic element sequence of an Anelloviridae family vector in bacteria using a site-specific recombinase, e.g., PhiC31. As shown in FIG. 79, a first plasmid comprising the genetic element sequence, flanked with recombinase recognition sites, e.g. attB and attP, is transformed into a bacterial cell such as E. coli. In one example, the bacterial cell is also transformed with a plasmid comprising an expression cassette for a site-specific recombinase (e.g., PhiC31) capable of recognizing the recognition sites in the vector plasmid. The recombinase is expressed and initiates site-specific recombination in the first plasmid. This results in the production of a minicircle comprising the genetic element sequence and a circular DNA molecule containing the excised vector backbone. In another instance, a bacterial cell that stably expresses a recombinase such as PhiC31 is transformed with a vector plasmid comprising the genetic element sequence, flanked with recombinase recognition sites, e.g. attB and attP. This can also produce minicircles containing the genetic element sequence. Minicircle produced in bacterial cells as described above can, in some instances, be isolated from the bacteria and can then be used for any of the applications described herein.

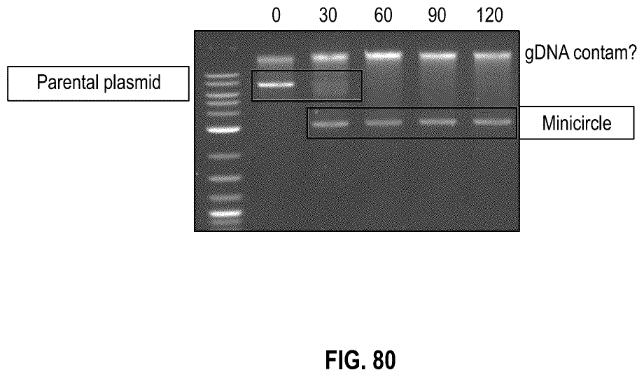

In one example, minicircles comprising the genetic element sequence of an Anelloviridae family vector were produced in bacterial cells as described above. Minicircle DNA was then isolated from the bacteria at 0, 30, 60, 90, or 120 minutes post-minicircle induction using standard miniprep methods and linearized with Xbal (FIG. 80). The expected parental plasmid length was 7146 bp, circularized backbone length 4078 bp, and minicircle length 3068 bp. As shown in FIG. 80, minicircle DNA was successfully isolated at 30-120 minutes post-induction.

### Example 42. Use of Minicircles for Production of Anelloviridae Family Vector Particles

This example describes the use of minicircles comprising genetic element sequences (e.g., produced as described above) for production of Anelloviridae family vector particles (see FIG. 79).

In some instances, the minicircles can be used to make particles in vitro (e.g., outside of a cell, e.g., in a cell-free system as described herein). In one example, the minicircles are produced in and then isolated from bacterial cells as described above. The isolated minicircles are then converted to cssDNA, for example, using any method described herein (e.g., by nicking and digesting one stand of the minicircle as described herein). The resultant cssDNAs can then be contacted with ORF1 molecules from a suitable Anelloviridae family virus and packaged into capsids comprising the ORF1 molecules, thereby producing particles.

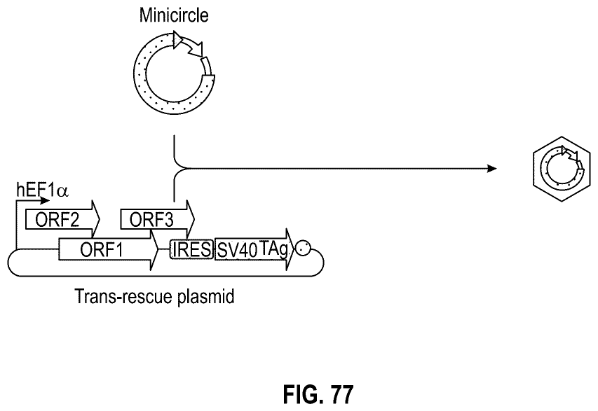

In some instances, the minicircles can be used to make particles in mammalian cells, e.g., MOLT-4 cells (see also FIG. 77). In one example, minicircles are transfected or virally transduced into MOLT-4 cells. A plasmid (e.g., a self-replicating rescue (SRR) plasmid) encoding ORF proteins from an Anelloviridae family virus (and, in some instances, and a SV Large T antigen expression cassette) are also introduced into the cells. The minicircle is then converted to cssDNA in the cell and then packaged by the capsid proteins expressed from the SRR to produce particles. The particles can be isolated and purified by any of the methods described herein.

### Example 43. Removing Homology Between the Vector and SRR Plasmids Reduces Production of Wild-Type (WT) Anelloviridae Family Viral Genomes

This example describes the modification of a self-replicating rescue (SRR) plasmid to improve the yield of Anelloviridae family vector genetic elements over wild-type (WT) Anelloviridae family viral genomes. In brief, a version of the SRR plasmid that removes an intron from the anellovirus NCR but retains the remainder of the NCR and the native kozak sequence, and a version of the SRR plasmid that only includes a native kozak sequence and does not contain any other portion of the Anelloviridae family viral NCR were created. These versions of the SRR plasmid remove most or all other homology between the SRR and the vector plasmid, thereby reducing or preventing homologous recombination that might otherwise lead to reconstitution of wild type viral genomes. As an example, SRR plasmid pRTx-3696 has the intron in the Ring19 NCR deleted but retains the remainder of the NCR and the native kozak sequence (see Table WX5). SRR plasmid pRTx-3856 contains the native kozak sequence but does not contain any other portion of the Ring19 NCR. Both versions of the SRR were able to produce DNAse-protected anellovectors without producing detectable wild type anellovirus.

### Materials and Methods

MOLT-4 cells were transfected with the plasmids as shown in Table 41X via electroporation.

TABLE 41X
Plasmids used to transfect MOLT-4 cells

| Condition | | |
|---|---|---|
| # | Description | Plasmid(s) |
| 1 | Vector alone | pRTx-2847 (genetic element |
| | | construct encoding eGFP, |
| | | Table Z1, SEQ ID NO: 2618) |
| 2 | Vector + R19ORF1KO | pRTx-2847 (genetic element |
| | | construct encoding eGFP) |
| | | pRTx-2848 (iCre-encoding |
| | | plasmid, Table Z5, SEQ ID |
| | | NO: 2622) |
| | | pRTx-3775 (R19 SRR with |
| | | ORF1 expression knocked out |
| | | by a premature stop codon in |
| | | the ORF1 coding sequence, |
| | | Table WX6, SEQ ID NO: 2006) |
| 3 | Vector + R19 SRR | pRTx-2847 (Table Z1) |
| | | pRTx-2848 (Table Z5) |
| | | pRTx-3525 (Table W1, SEQ ID |
| | | NO: 2000) |
| 4 | Vector + R19 | pRTx-2847 (Table Z1) |
| | Δintron 1 SRR | pRTx-2848 (Table Z5) |
| | | pRTx-3696 (Table WX5, SEQ ID |
| | | NO: 2005) |
| 5 | Vector + | pRTx-2847 (Table Z1) |
| | R19Δexon1Δintron1 | pRTx-2848 (Table Z5) |
| | SRR | pRTx-3856 (Table W2, SEQ ID |
| | | NO: 2002) |

Three days after transfection, cells were harvested and pelleted. Each pellet was resuspended with lysis mastermix (0.25% Triton X-100, 25 mM Tris pH 8.0, 150 mM NaCl, 2 mM MgCl2, 2× Proteinase inhibitor cocktail (Thermo Fisher Scientific catalog #78439), and 200 U of mSAN nuclease (ArticZymes AS catalog #70950-150)). The lysates were incubated at 37° C., shaking, for 1 hour. The lysates were clarified by centrifuging at 10,000×g for 20 minutes. The clarified lysates were transferred to new tubes without disturbing the pellet and stored at −80° C.

To prepare iodixanol linear gradients, 13 mL of 60% OptiPrep (Sigma-Aldrich catalog #D1556) was overlaid with 13 mL of 20% OptiPrep in 26.3-mL polycarbonate tubes, which were then spun at a 46-degree angle and a speed of 20 rpm for 16 minutes using Gradient Master (BioComp). Following the generation of the iodixanol linear gradient, 2 mL of iodixanol were removed from the top of each gradient, and 2 mL of thawed clarified lysate was added on top of the gradient. The sample-containing tubes were spun at 60,000 rpm and 20° C. for 1 hour using Type 70 Ti rotor (Beckman Coulter). 1-mL fractions were collected from the top of the tubes and transferred to a 96-well plate. The first 8 mL were pooled and discarded. 2 mL fractions were then collected and stored in a deep 96-well plate. 60 μl aliquots were dispensed into two working plates and stored at −80° C.

The plates were thawed and spun down. 5 μl of each sample was incubated with 20 U of DNAse I endonuclease (Thermo Fisher Scientific catalog #18047019) in a 20 μl reaction. The reaction was incubated at 37° C. for 30 minutes. Following DNase-treatment, each sample was subjected to Proteinase K (Fisher Scientific catalog #FEREOO491) and proteinase K buffer (1% SDS, 0.1M EDTA, 0.1M Tris pH 8.0, 0.1% Pluronic F-68). The reaction was incubated at 55° C. for 30 minutes, followed by proteinase K inactivation at 95° C. for 15 minutes and then held at 4° C. 4 μl of the 1:10 diluted DNase reaction was subjected to qPCR analysis in a 20-μl reaction using TaqMan™ Fast Advanced Master Mix (Thermo Fisher Scientific catalog #44-449-63) according to the manufacturer's protocol. Primer and probe sequences are listed in Table AA1.

### Results

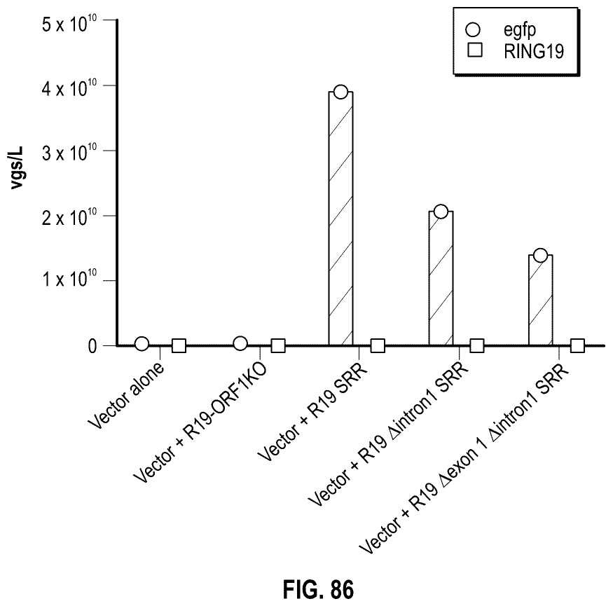

As shown in FIG. 86, no DNAse protected eGFP-encoding genomes were produced from cells transfected only with the genetic element construct encoding eGFP (“vector alone”; condition 1) or with the genetic element construct encoding eGFP, iCre-encoding plasmid, and an SRR with ORF1 expression knocked out (“vector+R19-ORF1KO”; condition 2) as expected. Also as expected, cells transfected with the genetic element construct encoding eGFP, iCre-encoding plasmid, and an intact Ring19 SRR (“vector+R19 SRR”; condition 3) produced DNAse protected eGFP-encoding genomes. Cells transfected with the genetic element construct encoding eGFP, iCre-encoding plasmid and either the SRR with the intron in the Ring19 NCR deleted but retaining the remainder of the NCR and the native kozak sequence (“vector+R19Δintron1 SRR”; condition 4) or the SRR with the native kozak sequence but lacking all other Ring19 NCR (“vector+R19Δexon1Δintron1 SRR”; condition 5) were also capable of producing DNAse protected eGFP-encoding genomes. WT Ring19 genomes were below the limit of quantification (LOQ) in all samples (FIG. 86).

### Example 44. Self-Replicating Rescue (SRR) Plasmid Configurations

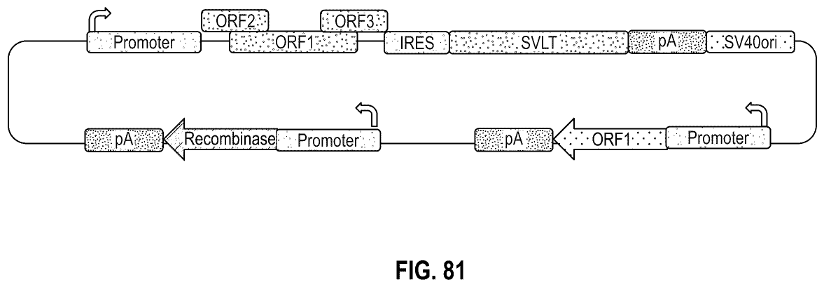

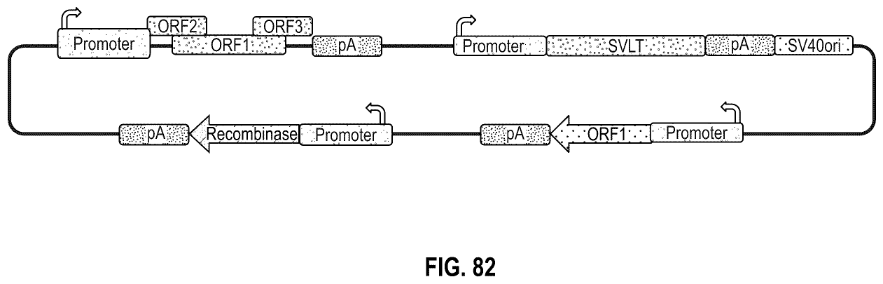

This example describes a series of exemplary configurations of cassettes that may be included in the self-replicating rescue (SRR) plasmid for use in the recombinase systems and methods described herein. Generally, the SRR contains at least one cassette comprising a promoter upstream of coding sequences for Anelloviridae family ORF1, ORF2, and ORF3 molecules (FIG. 81). In some instances, this cassette may also include an IRES sequence downstream of the Anelloviridae ORF coding region, which is in turn followed by an SV Large T antigen and an SV40 origin of replication driven by the same promoter as the Anelloviridae family ORFs (FIG. 81). In some instances, the SV Large T antigen and SV40 origin of replication are instead part of a separate cassette, with their expression driven by a separate promoter (FIG. 82). Such SRR plasmids can, in some instances, further include one or more additional cassettes. For example, as shown in FIGS. 81 and 82, SRR plasmids can include a separate cassette encoding an Anelloviridae family ORF1 molecule. Such SRR plasmids can, in certain instances, include a silencing mutation (e.g., a nonsense mutation, deletion, insertion, or frameshift mutation) in the ORF1 coding sequence of the cassette encoding the ORF1, ORF2, and ORF3 molecules. In other instances, the ORF1 coding sequence of the cassette encoding the ORF1, ORF2, and ORF3 molecules is intact (e.g., capable of expressing a functional ORF1 molecule). SRR plasmids can, in some instances, also include a separate cassette encoding a site-specific recombinase.

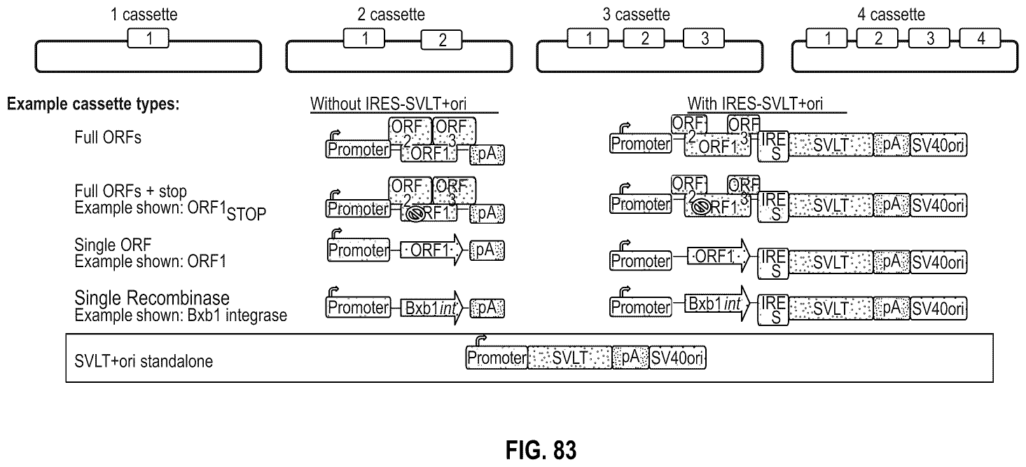

FIG. 83 summarizes how the cassettes of an SRR plasmid can be mixed and matched combinatorially. For example, an SRR plasmid may comprise 1, 2, 3, 4, or even more separate expression cassettes, in any order, provided that the SRRs contain at least one cassette expressing an SV40 Large T antigen and SV40 origin of replication, either through eg. IRES or as a standalone cassette expressed from a promoter. In some instances, a full ORF cassette may be included. In other instances, the cassette encoding the ORF1, ORF2, and ORF3 molecules comprises a mutant Anelloviridae family vector ORF sequence (for example, ORF1 with a silencing mutation, e.g., a premature stop codon), and a separate expression cassette expressing the ORF, e.g. ORF1, may be included. The SRR plasmid may also include additional cassettes, such as a cassette encoding a single Anelloviridae family vector ORF (e.g., an ORF1 molecule) instead of including all three, or a cassette encoding another transgene, such as a site-specific recombinase. The orientation of individual cassettes can be the same or different depending on the desired function. For example, all of the cassettes in a given SRR plasmid may be in the same orientation. Alternatively, one or more of the cassettes may be in the opposite orientation relative to other cassettes in the SRR plasmid.

### Example 45. Knocking Out Expression of ORF1 in the Self-Replicating Rescue (SRR) Plasmid does not Affect Expression of Other ORF Proteins and Splice Variants

This experiment shows that knocking out the expression of ORF1 in the SRR plasmid by adding a premature stop codon in the coding sequence of ORF1 does not affect the expression of other ORF proteins and splice variants from the SRR plasmid.

The Ring19 SRR, pRTx-3525, expresses all of the Ring19 ORF proteins, including ORF1, ORF2, ORF3, ORF1/1, ORF2/2, and ORF2/3 (see Table Z8). pRTx-3775 plasmid is the same as pRTx-3525, except that it contains two internal stop codons (TAA) in the Ring19 ORF1 coding sequence (see Table WX6). The stop codons were placed within the ORF1 coding sequence such that it would not affect the expression of ORF1/1 and other splice variants (see Table 43X). pRTx-4534 plasmid expresses only the Ring19 ORF1/1 in an SRR backbone (see Tables WX7 and 43X).

The Ring2 SRR, pRTx-3853, expresses all of the Ring2 ORF proteins, including ORF1, ORF2, ORF3, ORF1/1, ORF2/2, and ORF2/3 (see Table W3). pRTx-3776 plasmid is the same as pRTx-3853, except that it contains two internal stop codons (TAA) in the Ring2 ORF1 coding sequence (see Table WX8). The stop codons were placed within the ORF1 coding sequence such that it would not affect the expression of ORF1/1 and other splice variants (see Table 43X). pRTx-4463 plasmid expresses only the Ring2 ORF1/1 in an SRR backbone (see Tables WX9 and 43X).

TABLE 43X
Relevant ORF amino acid sequences in the SRR variants. * indicates stop codon.

Ring19 ORF1 sequence in pRTx-3775 plasmid
MPWYPRRRYPRRRYRWLRRWRARRPFRPRYRRRYWVRNYSRKRKL**ITTKEWQPKVIRKTHV
KGTYPLFLCTKHRINNNMIQYLDSIAPEHYYGGGGFSIMQFSLQALYEEFIKAKNWWTNTNCFLPLV
RYMGCSFKFYKTEFYDYIVLIERCYPLACTDEMYLSTQPSIMMLTRKCIFVPCKQNSKGKKPYKKVRV
RPPSQMTTGWHFSQDLANMPLVVLKTSVCSFDRYYTDSTAKSTTIGFKTLNTQTFRYHDWQEPPTTG
YKPQNLLWFYGAENGSPVDPNNTIVSNLIYLGGTGPYEKGTPIKTNISNYFSEPKLWGNIFHDDYTSG
TSPVFVTNKSPSEIKTAWNTIKDLTVKASGVFTLRTIPLWLPCRYNPFADKATNNKIWLVSIHSDHTEW
KPIDNPLLQRTDLPLWLLVWGWQDWQKKNQQTSQPDINYLTVISSPYISCYPKLDYYVLLDEGFWE
GHSTYIESITDSDKKHWYPKNRFQIETLNLIANTGPGTVKLRENQAAEGHMVYRFNFKLGGCPAPME
KICDPSKQSKYPIPNNQQQTTSLQSPENPIQTYLYDFDERRGLLTERATKRIKQDHTSEKTVLPFTGA
ATDLPILQTTSQEESSSEEEEEQQAEKKLLQLRRKQHRLRERILQLLDIQNT* (SEQ ID NO: 2776 and
SEQ ID NO: 2696, respectively)
Ring19 ORF1/1 sequence in pRTx-4534 plasmid
MPWYPRRRYPRRRYRWLRRWRARRPFRPRYRRRYWSPENPIQTYLYDFDERRGLLTERATKRI
KQDHTSEKTVLPFTGAATDLPILQTTSQEESSSEEEEEQQAEKKLLQLRRKQHRLRERILQLLDI
QNT* (SEQ ID NO: 2697)
Ring2 ORF1 sequence in pRTx-3776
MPYYYRRRRYNYRRPRWYGRGWIRRPFRRRFRRKRRVRPTYTTIPLKQWQPP**RTCYIKGQD
CLIYYSNLRLGMNSTMYEKSIVPVHWPGGGSFSVSMLTLDALYDIHKLCRNWWTSTNQDLPLVRYKG
CKITFYQSTFTDYIVRIHTELPANSNKLTYPNTHPLMMMMSKYKHIIPSRQTRRKKKPYTKIFVKPPPQ
FENKWYFATDLYKIPLLQIHCTACNLQNPFVKPDKLSNNVTLWSLNTISIQNRNMSVDQGQSWPFKIL
GTQSFYFYFYTGANLPGDTTQIPVADLLPLTNPRINRPGQSLNEAKITDHITFTEYKNKFTNYWGNPF
NKHIQEHLDMILYSLKSPEAIKNEWTTENMKWNQLNNAGTMALTPFNEPIFTQIQYNPDRDTGEDT
QLYLLSNATGTGWDPPGIPELILEGFPLWLIYWGFADFQKNLKKVTNIDTNYMLVAKTKFTQKPGTF
YLVILNDTFVEGNSPYEKQPLPEDNIKWYPQVQYQLEAQNKLLQTGPFTPNIQGQLSDNISMFYKFY
FKWGGSPPKAINVENPAHQIQYPIPRNEHETTSLQSPGEAPESILYSFDYRHGNYTTTALSRISQDWAL
KDTVSKITEPDRQQLLKQALECLQISEETQEKKEKEVQQLISNLRQQQQLYRERIISLLKDQ* (SEQ
ID NO: 2777 and SEQ ID NO: 2698, respectively)
Ring2 ORF1/1 sequence in pRTx-4463
MPYYYRRRRYNYRRPRWYGRGWIRRPFRRRFRRKRRIQYPIPRNEHETTSLQSPGEAPESILYS
FDYRHGNYTTTALSRISQDWALKDTVSKITEPDRQQLLKQALECLQISEETQEKKEKEVQQLIS
NLRQQQQLYRERIISLLKDQ* (SEQ ID NO: 59)
*indicates amino acid position corresponding to stop codon
Italicized portion is not expected to be translated

These plasmids were transfected into Expi293 cells and expression of the various ORF proteins were tested.

### Materials and Methods

Expi293 cells were transfected using PEIpro® (Polyplus (Sartorius)) with the plasmids and volumes used as shown in Table 43XX:

TABLE 43XX

| | Concentration | Plasmid Volume |
|---|---|---|
| Plasmid | (ng/μL) | Used (μL) |
| pRTx-3525 (Ring19 full SRR) | 1000 | 30 |
| pRTx-3775 (Ring19 w/ORF1 KO) | 1000 | 30 |
| pRTx-4534 (Ring19 ORF1/1 SRR) | 1000 | 30 |
| pRTx-3853 (Ring2 full SRR) | 2000 | 15 |
| pRTx-3776 (Ring2 w/ORF1 KO) | 1000 | 30 |
| pRTx-4463 (Ring2 ORF1/1 SRR) | 1000 | 30 |

In one tube (Tube A), 500 μL of Opti-MEM™ media (ThermoFisher) and the plasmid amounts shown in Table 43XX were mixed. In another tube (Tube B), 500 μL of Opti-MEM media and 90 μL of PEIpro® were mixed. Tubes A and B were each vortexed and incubated at room temperature for 10 minutes. The contents of Tube B was added into Tube A and vortexed. The mixture was incubated at room temperature for 20 minutes. The mixture was then added to 30 mL of Expi293 cells at a density of 1 E+06 cells/mL in a shake flask. The cells were incubated at 37° C. shaking for three days and then harvested for Western blot analysis. Anti-Ring19 ORF1, anti-Ring19 ORF2, anti-Ring2 ORF1, and anti-Ring2 ORF2 antibodies were used to visualize the respective proteins.

### Results

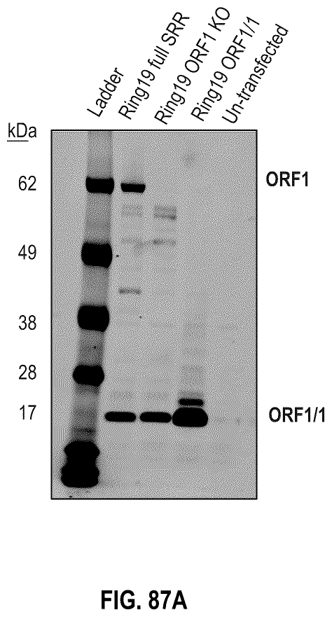

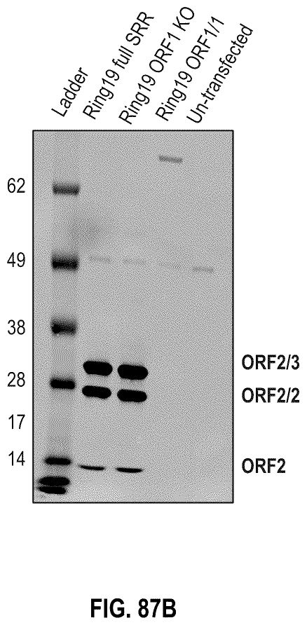

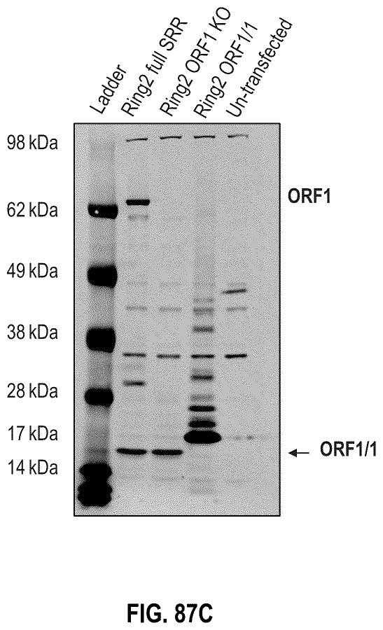

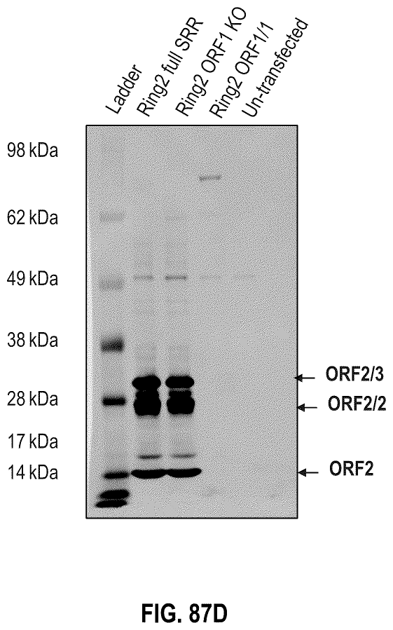

FIG. 87A shows a Western blot probed with an anti-Ring19 ORF1 antibody. As expected, the intact Ring19 SRR (pRTx-3525; “Ring19 full SRR”) expressed ORF1 and ORF1/1 (lane 2). The Ring19 SRR with the ORF1 expression knockout (pRTx-3775; “Ring19 ORF1 KO”) did not express the full-length ORF1 but did express ORF1/1 (lane 3). The Ring19 SRR expressing only ORF1/1 (pRTx-4534; “Ring19 ORF1/1”) showed only ORF1/1 and no full-length ORF1 expression (lane 4).

FIG. 87B shows a Western blot probed with an anti-Ring19 ORF2 antibody. As expected, the intact Ring19 SRR (pRTx-3525; “Ring19 full SRR”) expressed ORF2, ORF2/2, and ORF2/3 (lane 2). Similarly, the Ring19 SRR with the ORF1 expression knockout (pRTx-3775; “Ring19 ORF1 KO”) expressed ORF2, ORF2/2, and ORF2/3 (lane 3). Also as expected, the Ring19 SRR expressing only ORF1/1 (pRTx-4534; “Ring19 ORF1/1”) did not show expression of any of ORF2, ORF2/2, or ORF2/3 (lane 4).

FIG. 87C shows a Western blot probed with an anti-Ring2 ORF1 antibody. As expected, the intact Ring2 SRR (pRTx-3853; “Ring2 full SRR”) expressed ORF1 and ORF1/1 (lane 2). The Ring2 SRR with the ORF1 expression knockout (pRTx-3776; “Ring2 ORF1 KO”) did not express the full-length ORF1 but did express ORF1/1 (lane 3). The Ring2 SRR expressing only ORF1/1 (pRTx-4463; “Ring2 ORF1/1”) showed only ORF1/1 and no full-length ORF1 expression (lane 4).

FIG. 87D shows a Western blot probed with an anti-Ring2 ORF2 antibody. As expected, the intact Ring2 SRR (pRTx-3853; “Ring2 full SRR”) expressed ORF2, ORF2/2, and ORF2/3 (lane 2). Similarly, the Ring2 SRR with the ORF1 expression knockout (pRTx-3776; “Ring2 ORF1 KO”) expressed ORF2, ORF2/2, and ORF2/3 (lane 3). Also as expected, the Ring2 SRR expressing only ORF1/1 (pRTx-4463; “Ring2 ORF1/1”) did not show expression of any of ORF2, ORF2/2, or ORF2/3 (lane 4).

These results confirm that knocking out the expression of ORF1 in the SRR does not affect the expression of other ORF proteins and splice variants.

### Example 46. The Ring19 Genetic Element is Successfully Packaged by Other Ring ORF1 Molecules

This Example demonstrates that Ring ORF1 molecules of one Anellovector can package the genetic element of a second Anellovector.

### Materials and Methods

### Plasmids

### Genetic Element Construct Plasmid

The Ring19 genetic element construct, pRTX-2847, was designed with lox66 and lox 71 sites flanking a RING19 non-coding region (NCR) and a CMV::egfp::WPRE::bGH-pA payload cassette. The sequence for the construct is provided below.

TABLE Z1
Exemplary floxed Ring19 vector plasmid
for three-plasmid systems (pRTx-2847)

| | |
|---|---|
| Name | pRTx-2847 |
| Type | Plasmid |
| Description | pLox-Ring19ΔORF::fCMV_EGFP_{—} |
| | WPRE_bGH-pA-Rand100. |
| Length | 5452 bp |

(SEQ ID NO: 2699)
Annotations:

| Region/Element | Base range |
|---|---|
| Lox71 (loxP site) | 432-465 |
| GC-rich region | 529-640 |
| Initiator element | 813-828 |
| 5′ UTR conserved domain | 880-950 |
| Intron 1 | 892-979 |
| Full CMV promoter | 1004-1573 |
| Inert 5′ UTR | 1585-1619 |
| eGFP coding sequence | 1620-2339 |
| WPRE | 2343-2934 |
| bGHpA terminator sequence | 2939-3166 |
| 100 bp random stuffer sequence | 3167-3266 |
| Lox66 (loxP site) | 3271-3304 |
| LacI repressor binding site (complement) | 3384-3406 |
| Lac operon operator (lacO) | 3386-3402 |
| Lac operon promoter (complement) | 3410-3440 |
| C-tag | 3418-3429 |
| E coli catabolite activator protein | 3455-3476 |
| binding site (complement) | |
| Origin of replication (pUC origin) | 3705-4378 |
| (complement) | |
| Kanamycin resistance (KanR) CDS | 4530-5339 |
| (complement) | |

### Self-Replicating Rescue (SRR) Plasmid Expressing Anellovirus ORF1

Anelloviral ORF1 was expressed from a self-replicating rescue (SRR) plasmid. Table X lists each of the SRR plasmids used for the expression of various Anelloviral ORF1.

TABLE X
Self-replicating rescue plasmids used to
produce cross-packaged Anellovectors.

| Plasmid | Anellovirus ORF1 |
|---|---|
| pRTx-3525 | Ring19 |
| pRTx-3853 | Ring2 |
| pRTx-3861 | Ring25 |
| pRTx-3905 | Ring9 |
| pRTx-3906 | Ring34 |
| pRTx-4177 | RING10 |
| pRTx-4185 | RING161 |
| pRTx-4192 | YYBYFW |

### Cre Expression Plasmid

pRTX-2848 expresses iCre from the CMV promoter. The sequence of this construct is provided below.

TABLE Z5
Exemplary Cre recombinase expression plasmid (pRTx-2848)

| | |
|---|---|
| Name | pRTx-2848 |
| Type | Plasmid |
| Description | CMV_iCre_pcDNA3.1(+). |
| Length | 6473 bp |

(SEQ ID NO: 2700)
Annotations:

| Region/Element | Base range |
|---|---|
| CMV enhancer | 235-614 |
| CMV promoter | 615-818 |
| T7 RNA polymerase promoter | 863-880 |
| iCre coding sequence | 964-2019 |
| bGHpA terminator sequence | 2070-2293 |
| Origin of replication | 2962-3097 |
| SV40 polyA sequence | 4146-4267 |
| Lac operon operator (lacO) | 4340-4356 |
| Lac operon promoter (complement) | 4364-4394 |
| C-tag | 4372-4383 |
| Catabolite activator protein binding site | 4409-4430 |
| Origin of replication (complement) | 4718-5306 |
| Ampicillin resistance gene promoter | 6338-6442 |
| (complement) | |

### Cross Packaged Anellovector Production

Anellovectors were produced in MOLT4 cells by triple electroporation of a genetic element construct plasmid, a Cre expression plasmid, and an SRR plasmid. Transfected cultures were harvested on day 3 post-electroporation, pelleted by centrifugation, and incubated in a M-SAN/Triton X-100 lysis buffer. Cell lysates were loaded onto an iodixanol linear gradient for separation of vector particles, taking fractions around the expected density of Anelloviruses. Packaged vector genomes were titered from collected fractions by DNase digestion to remove any unencapsidated DNA, and quantitated by qPCR against eGFP. Controls included MOLT4 cells transfected with Ring19 genetic element construct (pRTX-2847) alone and MOLT4 cells transfected with Ring19 genetic element construct (pRTX-2847), Ring19 SRR (pRTx-3525), and pRTx-2848 (Cre expression vector).

### Results

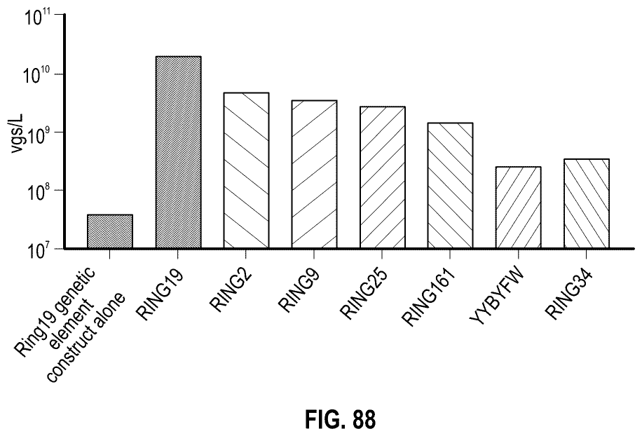

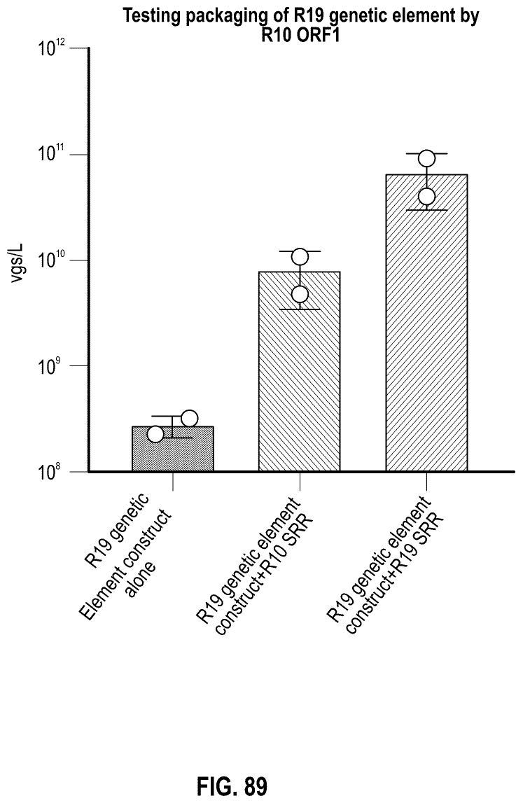

As shown in FIG. 88, ORF1 from Ring19, Ring2, Ring9, Ring25, Ring161, YYBYFW, and Ring 34 were capable of cross packaging a Ring19 genetic element and producing Anellovectors at levels greater than when cells were transfected with a Ring19 genetic element construct alone. FIG. 89 also shows that ORF1 from Ring10 was capable of cross packaging a Ring19 genetic element and producing Anellovectors at levels greater than when cells were transfected with Ring19 genetic element construct alone. These studies demonstrate that Anelloviral ORF1 proteins from one Anellovirus are capable of packaging a genetic element from a different Anellovirus.

### Example 47: In Vivo Transduction of Cross-Packaged R2 ORF1 and Ring19 Genetic Element Anellovector

This example demonstrates in vivo transduction using a cross-packaged Anellovector.

### Materials & Methods

### Vectors:

AAV2-fCMV-eGFP is an adeno-associated virus based on the plasmid pRTx-2770 with a payload comprising from the 5′ to 3′ direction an AAV2 ITR, Ring2 5′ NCR, CMV promoter, eGFP, SV40 pA, Ring2 3′ NCR, and AAV2 ITR (Table Z6). AAV2-fCMV-eGFP was prepared by Packgene Biotech Inc. and was diluted in sterile 1×PBS.

Ring2-fCMV-eGFP (an Anellovector with Ring2 ORF1 encapsulating Ring19-eGFP genetic element) and Ring19-fCMV-eGFP (an Anellovector with Ring19 ORF1 encapsulating Ring19-eGFP genetic element) were prepared as described below.

TABLE Z6
pRTx-2770

| | |
|---|---|
| Name | pRTx-2770 |
| Type | Plasmid |
| Length | 5128 bp |

(SEQ ID NO: 2701)
Annotations:

| Region/Element | Base range |
|---|---|
| E. coli CAP protein binding site | 130 . . . 151 |
| Lac operon promoter | 166 . . . 196 |
| LacI repressor protein binding site | 204 . . . 220 |
| AAV2 inverted terminal repeat | 269 . . . 409 |
| Ring2 5′NCR | 421 . . . 844 |
| CMV enhancer | 881 . . . 1260 |
| Full CMV promoter | 896 . . . 1465 |
| CMV promoter | 1261 . . . 1464 |
| Inert 5′ UTR | 1477 . . . 1507 |
| Kozak sequence | 1506 . . . 1515 |
| eGFP coding sequence | 1512 . . . 2231 |
| SV40 poly(A) sequence | 2262 . . . 2383 |
| Ring2 3′NCR | 2411 . . . 2575 |
| AAV2 inverted terminal repeat | 2589 . . . 2729 |
| AmpR promoter | 3246 . . . 3350 |
| Beta lactamase ampicillin resistance coding sequence | 3351 . . . 4211 |
| Gigh-copy-number ColE1/pMB1/pBR322/pUC origin | 4382 . . . 4970 |
| of replication | |

Exemplary eGFP Amino Acid Sequence (e.g., Encoded by the Nucleotides 1512-2231 of Table Z6):

(SEQ ID NO: 2650)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

### Exemplary Beta Lactamase Amino Acid Sequence (e.g., Encoded by of Nucleotides 3351-4211 of Table Z6):

(SEQ ID NO: 2702)
MSIQHFRVALIPFFAAFCLPVFAHPETLVKVKDAEDQLGARVGYIELDL
NSGKILESFRPEERFPMMSTFKVLLCGAVLSRIDAGQEQLGRRIHYSQN
DLVEYSPVTEKHLTDGMTVRELCSAAITMSDNTAANLLLTTIGGPKELT
AFLHNMGDHVTRLDRWEPELNEAIPNDERDTTMPVAMATTLRKLLTGEL
LTLASRQQLIDWMEADKVAGPLLRSALPAGWFIADKSGAGERGSRGIIA
ALGPDGKPSRIVVIYTTGSQATMDERNRQIAEIGASLIKHW

### Parental Cell Culture

MOLT-4 cells were obtained from the National Cancer Institute. Cells were scaled-up and maintained in suspension culture in complete growth medium (Gibco's RPMI 1640 with 10% fetal bovine serum [FBS], supplemented with 1 mM sodium pyruvate, Pluronic F-68 [0.1%], and 2 mM L-glutamine) at 37° C. with 5% CO₂. Cells were seeded into shake flasks (2-L, flat-bottomed, Erlenmeyer flask), each with a working volume of 800 mL, at a density of 0.1 E+06 viable cells/mL and cultured in an orbital shaker (New Brunswick Innova 2100, 19-mm circular orbit) at 37° C. and 90 rpm with >85% relative humidity (RH) for 4 days.

### Transfection of MOLT-4 Cells

Each viral vector was produced using a distinct combination of three plasmids, via electroporation into MOLT-4 cells. MOLT-4 cells were transfected via electroporation with the plasmids shown in Table E0:

TABLE E0
Plasmids used to produce an Anellovector with Ring19
ORF1 encapsulating a Ring19 genetic element (R19-
eGFP) and an Anellovector with Ring2 ORF1 encapsulating
a Ring19 genetic element (R2-eGFP).

| Anellovector | Plasmids |
|---|---|
| Ring19-fCMV-eGFP (“R19-eGFP”) | pRTx-2847 (R19-eGFP vector) |
| | pRTx-2848 (iCre plasmid) |
| | pRTx-3525 (R19 SRR) |
| Ring2-fCMV-eGFP (“R2-eGFP”) | pRTx-2847 (R19-eGFP vector) |
| | pRTx-2848 (iCre plasmid) |
| | pRTx-3853 (R2 SRR) |

For each of the viral vector production culture preps, 5 E+08 MOLT-4 cells were pelleted and resuspended in Opti-MEM I Reduced Serum Medium. 800 μg of the plasmids were added to each of the resuspended cells and electroporated using a MaxCyte STx electroporator and CL1.1 electroporator assemblies (MaxCyte catalog #SCL1). Each batch of electroporated cells were then transferred to separate flasks containing pre-warmed complete growth medium. Transfected cells were allowed to incubate at 37° C. with 5% CO₂ and harvested via centrifugation 72 hours post-electroporation.

### Cell Lysis

Cell pellets were resuspended in lysis buffer containing 50 mM Tris pH 8.0, 0.5% Triton-X100, 100 mM NaCl, 100× Halt protease inhibitor cocktail (Thermo Fisher Scientific catalog #78439), and 200 U of mSAN nuclease (ArcticZyme catolog #NC1920045). The cell lysates were clarified by centrifugation at 12,500×g for 30 minutes at 4° C.

### Isopycnic Centrifugation

To prepare iodixanol linear gradients, 13 mL of 60% OptiPrep (Sigma-Aldrich catalog #D1556) was overlayed with 13 mL of 20% OptiPrep in 26.3-mL polycarbonate tubes, which were then spun at a 46-degree angle and a speed of 20 rpm for 16 minutes using Gradient Master (BioComp). Following the generation of the iodixanol linear gradient, 2 mL of iodixanol were removed from the top of each gradient, and 2 mL of clarified lysate was added on top of the gradient. The sample-containing tubes were spun at 347,000×g and 20° C. for 3 hours using Type 70 Ti rotor (Beckman Coulter). 1-mL fractions were collected from the top of the tubes and transferred to a 96-well 2.2 ml capacity plate. Each fraction was then subjected to a DNase-protected qPCR assay as described below.

### DNase-Protection Assay

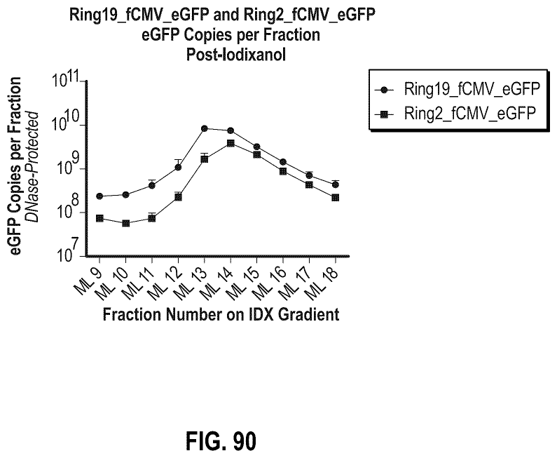

5 μl of each sample to be titered was incubated with 20 U of DNAse I endonuclease (Thermo Fisher Scientific catalog #18047019) in a 20 μl reaction. The reaction was incubated at 37° C. for 30 minutes. Following DNase-treatment, each sample was subjected to Proteinase K (Fisher Scientific catalog #FEREOO491) and proteinase K buffer (1% SDS, 0.1M EDTA, 0.1M Tris pH 8.0, 0.1% Pluronic F-68). The reaction was incubated at 37° C. for 30 minutes, followed by proteinase K inactivation at 95° C. for 15 minutes. 4 μl of the 1:10 diluted DNase reaction was subjected to qPCR analysis in a 20-μl reaction using TaqMan Fast Universal PCR Master Mix (Thermo Fisher Scientific catalog #44-449-63) according to the manufacturer's protocol. Primer and probe sequences are listed in Table E10. FIG. 90 shows the post-Iodixanol DNase-Protection Assay of Ring19_fCMV_eGFP and Ring2_fCMV_eGFP Material.

### Concentration of Material

Fractions of interest were determined based on the viral titer. The pooled iodixanol fractions were then diluted 1:50 in formulation buffer (1×DPBS, 0.001% Pluronic F-68) and concentrated using Vivaspin 20 100K MWCO centrifugal filter units (Fisher Scientific catalog #1455810).

### DNase-Protection Assay on Final Material

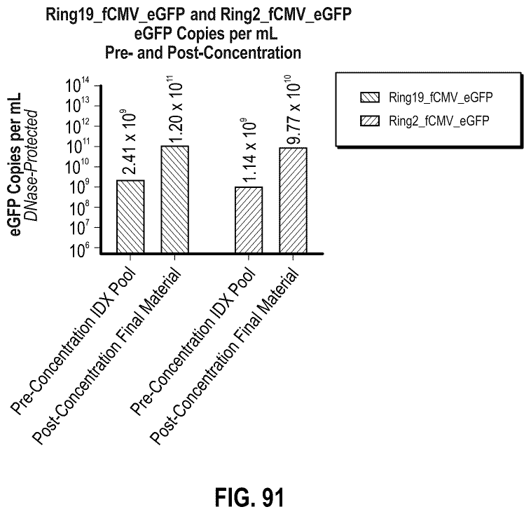

5 μl of the sample to be titered was incubated with 20 U of DNAse I endonuclease (Thermo Fisher Scientific catalog #18047019) in a 20-μl reaction. The reaction was incubated at 37° C. for 30 minutes. Following DNase-treatment, each sample was subjected to Proteinase K (Fisher Scientific catalog #FERE00491) and proteinase K buffer (1% SDS, 0.1M EDTA, 0.1M Tris pH 8.0, 0.1% Pluronic F-68). The reaction was incubated at 37° C. for 30 minutes, followed by proteinase K inactivation at 95° C. for 15 minutes. 4 μl of the 1:10 diluted DNase reaction was subjected to qPCR analysis in a 20-μl reaction using TaqMan Fast Universal PCR Master Mix (Thermo Fisher Scientific catalog #44-449-63) according to the manufacturer's protocol. Primer and probe sequences are listed in Table E10. FIG. 91 shows the pre- and Post-Concentration DNase-Protection Assay of Ring19_fCMV_eGFP and Ring2_fCMV_eGFP Material.

### Endotoxin Test on Final Material

2.241 of the sample was diluted 1:50 in formulation buffer (1×DPBS, 0.001% Pluronic F-68) and the sample was subjected to the LAL detection test (Charles River) according to the manufacturer's protocol.

### SDS-PAGE and Coomassie Stain on Final Material

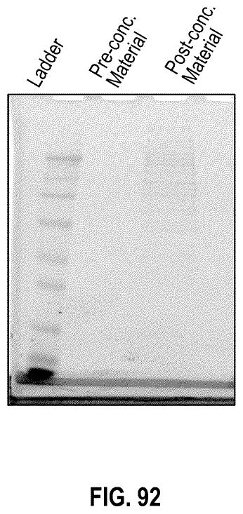

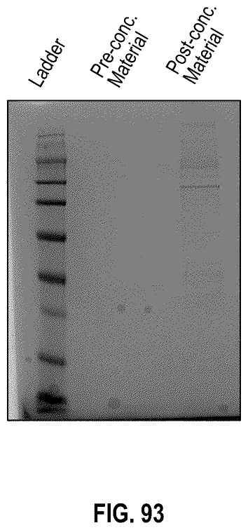

2 μl of the sample was diluted 1:10 in formulation buffer (1×DPBS, 0.001% Pluronic F-68) and the sample was mixed 1:1 with loading dye and Bolt sample reducing agent (Thermo Fisher Scientific catalog #B0009), followed by boiling at 95° C. for 10 minutes. Proteins were separated on Bolt 4-12% Bis-Tris gel in 1× Bolt MOPS SDS running buffer (Thermo Fisher Scientific catalog #B0001). Separated proteins were stained using InstantBlue Coomassie Protein Stain (ABCAM catalog #ab119211) according to the manufacturers protocol. Following the stain, the gel was washed three times with diH2O. The stained gel was visualized using Chemidoc Imaging System (BioRad) as shown in FIG. 92 and FIG. 93.

### Bicinchonic Acid Assay (BCA)

2 μl of the sample was diluted 1:10 in formulation buffer (1×DPBS, 0.001% Pluronic F-68) and the sample was subjected to the Pierce BCA Protein Assay Kit (ThermoScientific catalogue #23227) according to the manufacturer's protocol.

### Care and Use of Animals.

All mouse studies were approved and governed by the Ring Therapeutics Institutional Animal Care and Use Committee. Female C57Bl/6J mice 9-12 weeks of age were obtained from Jackson Laboratories for use in these studies.

### Subretinal Injections

Pupils were first dilated with one to two drops of 1% tropicamide/2.5% phenylephrine HCl (Tropi-Phen, Pine Pharmaceuticals). The mouse was subsequently anesthetized using an intraperitoneal injection of a ketamine/xylazine cocktail (100/10 mg/kg) (Patterson Veterinary Supply, Inc.). One or two drops of 0.5% proparacaine (McKesson Medical-Surgical Inc.) were applied to the eye. An incision approximately 0.5 mm in length was made with a micro scalpel 1 mm posterior to the nasal limbus. A 33-g blunt-ended needle on a 5-μl Hamilton syringe was inserted through the scleral incision, posterior to the lens, toward the temporal retina until resistance was felt. 1 μl of either 1×PBS, virus, or vector containing 0.1% of sodium fluorescein (AK-Fluor 10%, Akorn) was then injected slowly into the subretinal space. The eye was examined and the success of the subretinal injection was confirmed by visualizing the fluorescein-containing bleb through the dilated pupil with a Leica M620 TTS ophthalmic surgical microscope (Leica Microsystems, Inc.). Eyes with significant hemorrhage or leakage of vector solution from the subretinal space into the vitreous were excluded from the study. After the procedure, 0.3% tobramycin ophthalmic ointment (Tobrex, McKesson Medical-Surgical Inc.) was applied to each treated eye and the mouse was allowed to recover from the anesthesia prior to being returned to its cage in the housing room.

### Tissue Harvest for Flatmount Analysis

Mouse eyes were dissected at the indicated time point following subretinal injections (n=2 for the given timepoint). After enucleation, whole eyes were placed into 4% paraformaldehyde (PFA) solution for fixation. The fixed eyes were transferred to 1×PBS, after 2 hours.

The fixed eye was placed into a petri dish with 1×PBS under the Leica M80 stereomicroscope (Leica Microsystems Inc.). The cornea and lens were removed and discarded. The neural retina was separated from the posterior eye cup (PEC) and discarded. 8 radial cuts were evenly placed throughout the PEC, perpendicular to the optic nerve head. The PEC was then moved onto a glass microscope slide and arranged in a flat, even position. 1-2 drops of mounting media (ProLong Glass Antifade Mountant, Invitrogen) were dispensed over the tissue. A glass cover slip was placed over the mountant and slight pressure was applied to remove bubbles and further flatten the tissue. The slides were left for a minimum of 2 hours to cure before imaging.

### Flatmount Imaging

Once cured, images of the PEC flatmount slides were collected from each group using the EVOS M7000 microscope (Life Technologies Corporation). Images were captured in the green channel (EVOS Light Cube, GFP 2.0, Life Technologies Corporation) and the red channel (EVOS Light Cube, Texas Red 2.0, Life Technologies Corporation) at 20× magnifications, utilizing congruent exposures for each image.

### Tissue Harvest for DNA and RNA Extraction

Mouse eyes were dissected at indicated time points following subretinal injections (n=8 for the given timepoint). After enucleation, the retina and posterior eyecup (PEC) were separated and processed individually. These tissues were collected in 2 mL reinforced tubes (SPEX Sample Prep) containing 5 mm stainless steel beads (Qiagen, LLC) and flash-frozen immediately. They were stored at −80° C. until homogenization.

### DNA Isolation

Frozen tissue samples were lysed with an automated tissue homogenizer (Geno/Grinder SPEX Sample Prep) in Buffer ATL (Qiagen, USA) and proteinase K (Qiagen, USA) at 1250 rpm for two 30 second rounds. Homogenized tissues were digested on heat block at 56C for about 4 hours. Genomic DNA was precipitated with Buffer AL (Qiagen, USA) and Ethanol, then isolated with Qiagen DNeasy 96 Blood & Tissue Kit. Isolated DNA was quantified using NanoDrop 8000 Spectrophotometer (Thermofisher, USA).

qPCR

Genomic DNA was assayed by qPCR on the QuantStudio 5—Real-Time PCR System (Thermo Fisher, USA) using TaqMan Gene Expression Master Mix (Thermofisher, USA). The sequence detection primers and FAM custom probes that were used in this study were synthesized by Integrated DNA Technologies, USA. eGFP, wild-type Ring2 and wild-type Ring19 primer/probe sequences are in Table E10.

All reactions including the DNA samples and different dilutions of a known quantity of the linearized eGFP and R19 plasmid standards were run in triplicate on the same plate. The standard curve method was used to calculate the amount of viral/vector DNA, which was normalized with the total amount of genomic DNA for each sample (quantified using a nanodrop as described above).

### RNA Isolation

Frozen tissue samples were lysed with an automated tissue homogenizer (Geno/Grinder SPEX Sample Prep, USA) in QIAzol lysis reagent (Qiagen, USA) at 1250 rpm for two 30 second rounds. RNA was then isolated in aqueous phase by the addition of Phenol Chloroform (Thermofisher, USA) and centrifugation at 6000 rpm for 15 minutes at 4 C. The upper aqueous phase was transferred into a fresh S-block (Qiagen, USA). RNA was precipitated with the addition of one volume of 70% Ethanol and isolated with the Qiagen RNeasy 96 kit. RNA concentration was then quantified via Qubit RNA High Sensitivity Assay Kit (Thermofisher, USA).

### RT-qPCR

RNA was reverse transcribed into cDNA using Thermofisher SuperScript IV VILO Master Mix with ezDNase according to manufacturer's protocol with additional reactions included for No Reverse Transcriptase (NRT) controls. cDNA was diluted with nuclease-free water to ensure enough volume to run all qPCR assay reactions necessary.

cDNA was assayed by qPCR on the QuantStudio 5—Real-Time PCR System (Thermo Fisher, USA) using TaqMan Gene Expression Master Mix (Thermofisher, USA). The sequence detection primers and FAM custom probes that were used in this study were synthesized by Integrated DNA Technologies and can be found in Table E10.

Mouse GAPDH was used as an endogenous control in duplicates (ThermoFisher, USA) for both RT and no RT samples. All reactions including the cDNA samples and different dilutions of a known quantity of the linearized eGFP plasmid standards were run in triplicate on the same plate. The standard curve method was used to calculate the quantities of mRNA copies and was normalized using the no RT signal and the total amount of RNA input into the RT reaction.

### One-Step RT-ddPCR

RNA was diluted in nuclease-free water and combined with the reagents from the One Step RT-ddPCR Advanced Kit for Probes (Bio-Rad, USA; Catalog #1864022) and eGFP primer/probe set with final primer concentrations of 900 nM and probe concentrations of 250 nM to measure transgene expression. After the RT-ddPCR reaction setup, each reaction was converted to droplets using the Automated Droplet Generator (Bio-Rad, USA) according to the manufacturer's instructions. After the droplet generation, the droplets were subjected to endpoint PCR thermocycling with the following cycling conditions: 1 cycle of 48 C for 1 hour for reverse transcription followed by 1 cycle of 95 C for 10 mins; 40 cycles of 95 C for 30 sec, 60 C for 1 min; and 1 cycle of 98 C for 10 min and finally a 4 C hold. The cycled plate was then transferred to the QX200 Droplet Reader (Bio-Rad, USA) and analyzed using QX Manager Software (Bio-Rad, USA).

TABLE E10
Primers and probes designed to quantify eGFP, wild-type Ring2 (WT R2),
and wild-type Ring19 (WT R19) genomic sequences.

| Target | Label | Sequence (5′→3′) |
|---|---|---|
| eGFP | Forward Primer | GAACCGCATCGAGCTGAA (SEQ ID NO: |
| | | 2690) |
| | Reverse Primer | TGCTTGTCGGCCATGATATAG (SEQ ID |
| | | NO: 2691) |
| | Probe (FAM) | ATCGACTTCAAGGAGGACGGCAAC |
| | | (SEQ ID NO: 2692) |
| WT R2 | Forward Primer | GAAGCCCACCAAAAGCAATT (SEQ ID |
| | | NO: 2703) |
| | Reverse Primer | AGTTCCCGTGTCTATAGTCGA (SEQ ID |
| | | NO: 2704) |
| | Probe (FAM) | ACTTCGTTACAGAGTCCAGGGG (SEQ |
| | | ID NO: 2705) |
| WT R19 | Forward Primer | GGATTTTGGGAGGGTCACTC (SEQ ID |
| | | NO: 2693) |
| | Reverse Primer | TACAGTTCCTGGACCTGTGT (SEQ ID |
| | | NO: 2694) |
| | Probe (FAM) | ACACTGGTACCCTAAAAATAGATTTCA |
| | | (SEQ ID NO: 2695) |

### Results

### Infectivity of Cross-Packaged Anellovector R2-fCMV-eGFP (“R2-eGFP”)

The prepared virus preparations were injected into mice subretinally as shown in Table E2. Mice were injected with PBS, R2-eGFP (Ring2 ORF1 encapsulating the Ring19-eGFP genetic element), R19-eGFP (Ring19 ORF1 encapsulating the Ring19-eGFP genetic element), or dose-matched AAV2-eGFP. 21 days after injection, the posterior eye cup (PEC) and retina were collected and processed separately.

TABLE E2
Experimental design for testing in vivo infectivity of cross-packaged Anellovectors.

| Group | Treatment | Dose/eye (vg) | Route (volume) | N (eyes) | Terminal Day |
|---|---|---|---|---|---|
| 1 | PBS | 0 | Subretinal (1 ul) | 12 | 21 |
| 2 | R2-fCMV-eGFP (“R2-eGFP”) | 1.2e+7 | Subretinal (1 ul) | 12 | 21 |
| 3 | R19-fCMV-eGFP (“R19-eGFP”) | 1.2e+7 | Subretinal (1 ul) | 12 | 21 |
| 4 | AAV2-fCMV-eGFP (“AAV2-eGFP”) | 1.2e+7 | Subretinal (1 ul) | 11 | 21 |

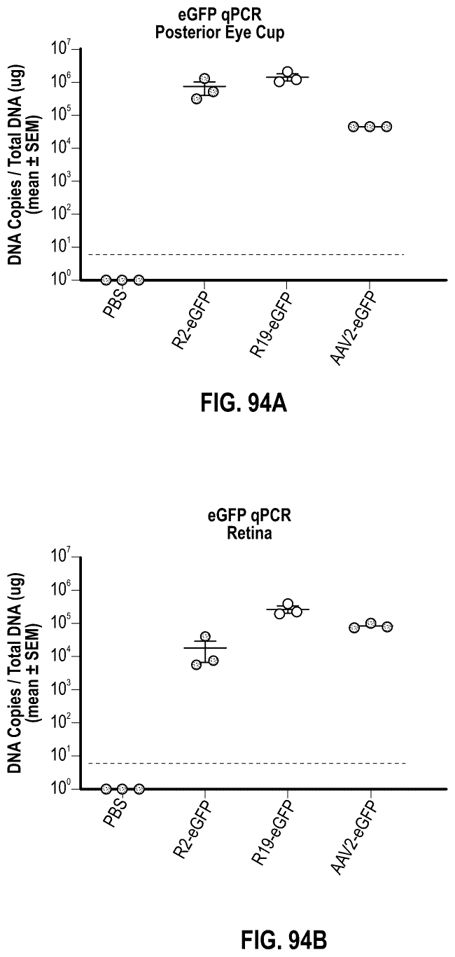

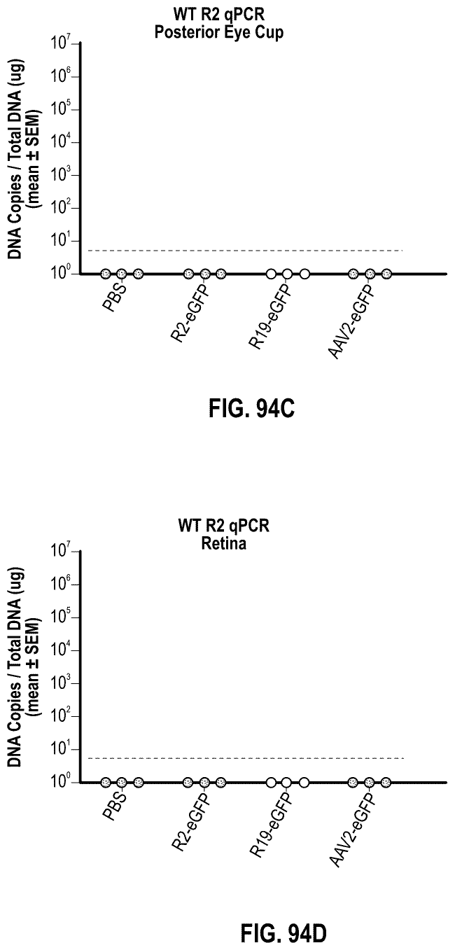

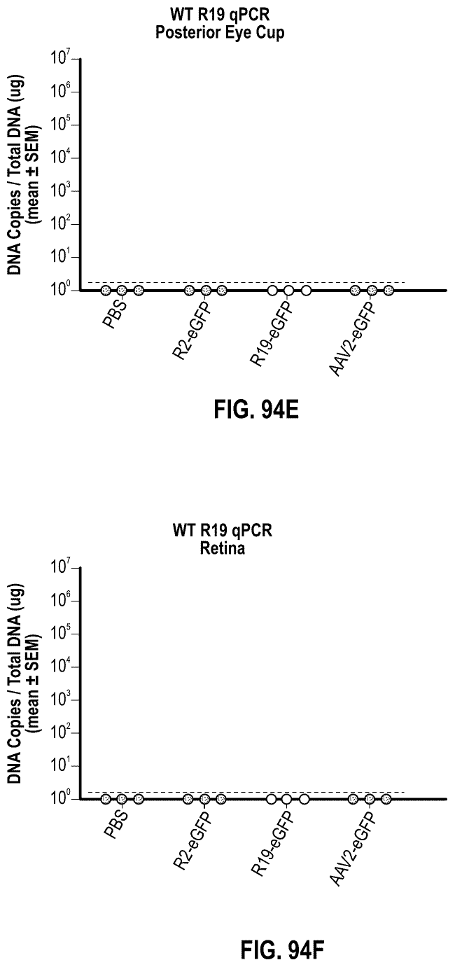

eGFP genomes were detected by qPCR in the PEC and retina transduced with the cross packaged R2-eGFP Anellovector (R2 ORF1 encapsulating a R19-eGFP genome), R19-eGFP Anellovector (R19 ORF1 encapsulating a R19-eGFP genome), and AAV2-eGFP (FIG. 94A and FIG. 94B). The level of eGFP genomic DNA was greater in the PEC transduced with either R2-eGFP or R19-eGFP Anellovectors compared to PEC transduced with dose-matched AAV2-eGFP (FIG. 94A). Furthermore, the level of eGFP genomic DNA in PEC transduced with R2-eGFP was similar to the level of eGFP genomic DNA transduced with R19-eGFP Anellovector (FIG. 94A), demonstrating that cross-packaged Anellovectors can transduce genetic material in vivo at a level similar to non-cross-packaged Anellovectors. Wild type (WT) Ring2 and Ring19 genomes were not detected by qPCR in either the PEC or retina at day 21 (FIGS. 94C-94F), demonstrating that Anellovectors can be produced efficiently without wild type contamination.

Administration of Cross Packaged Anellovector R2-fCMV-eGFP (“R2-eGFP”) Induces eGFP mRNA Expression

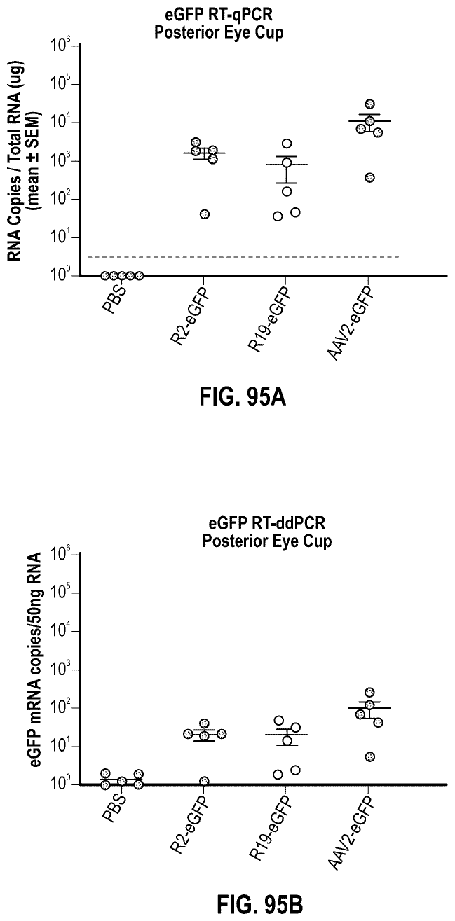

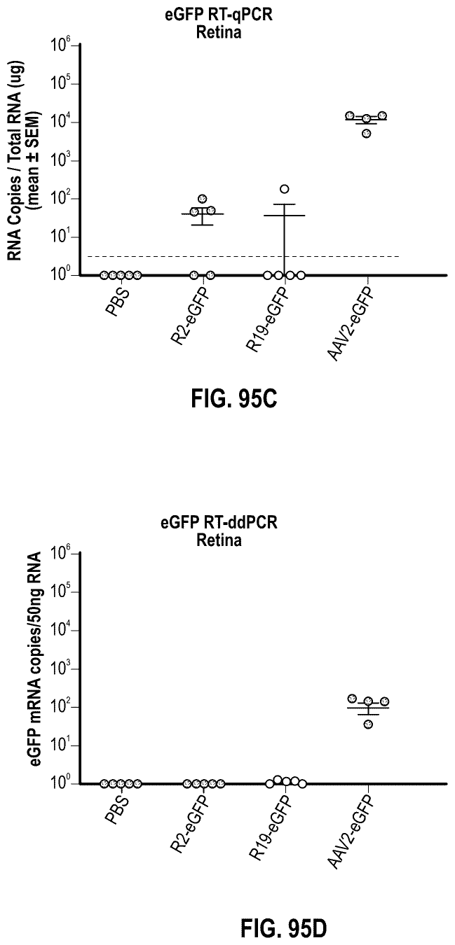

To determine whether cross packaged Anellovector R2-eGFP (Ring2 ORF1 encapsulating the Ring19-eGFP genetic element) can induce eGFP expression, RNA was collected from PEC and retina tissue at 21 days after infection (n=5 eyes per group). eGFP mRNA was then quantified by RT-qPCR. eGFP mRNA was detected in both the PEC and the retina 21 days after infection with R2-eGFP (Ring2 ORF1 encapsulating the Ring19-eGFP genetic element), R19-eGFP (Ring19 ORF1 encapsulating the Ring19-eGFP genetic element), and AAV2-eGFP, while no eGFP mRNA was detected in the PBS control group (FIG. 95A and FIG. 95C). The levels of eGFP mRNA in the PEC and retina infected with R2-eGFP Anellovector were similar to the levels of eGFP mRNA in the PEC and retina infected with R19-eGFP Anellovector (FIG. 95A and FIG. 95C). One-step RT-ddPCR was further conducted (FIG. 95B and FIG. 95D) and the presence of eGFP mRNA was confirmed in the PEC.

Administration of Cross Packaged Anellovector R2-fCMV-eGFP (“R2-eGFP”) Results in eGFP Protein Expression

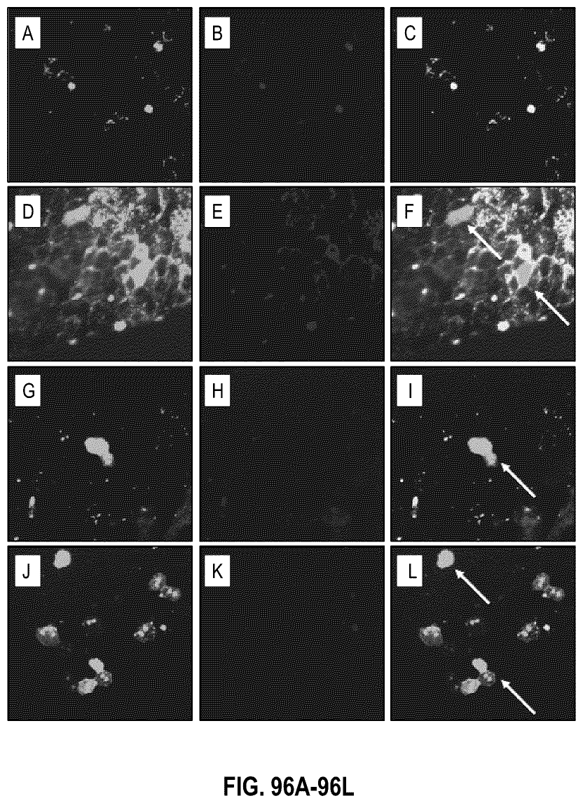

To determine whether administration of R2-eGFP Anellovector (Ring2 ORF1 encapsulating the Ring19-eGFP genetic element) can produce functional eGFP protein in vivo, PEC was collected at day 21 after infection for fixation and flatmount imaging (n=3 eyes per group). To determine true eGFP expression, representative sections of the tissue were imaged in both the eGFP and Texas red channels. Overlapping these channels showed that any area where only eGFP signal was detected showed true eGFP expression, while any signal that overlapped between the eGFP and Texas red channels was caused by red blood cell autofluorescence. FIGS. 96A-96C show the eGFP channel, Texas red channel, and overlap of the two channels for the PBS control group, respectively. No eGFP positive cells were found in the PBS negative control group. FIGS. 96D-96F show the eGFP channel, Texas red channel, and overlap of the two channels for the R2-eGFP group (Ring2 ORF1 packaging the Ring19-eGFP genetic element), respectively. Cells expressing eGFP protein are shown by the white arrows in FIG. 96F. FIGS. 96G-96I show the eGFP channel, Texas red channel, and overlap of the two channels for the R19-eGFP group (Ring19 ORF1 packaging the Ring19-eGFP genetic element), respectively. An example of a cell expressing eGFP protein is shown by the white arrow in FIG. 96F. FIGS. 96J-96L show the eGFP channel, Texas red channel, and overlap of the two channels for the dose-matched AAV2-eGFP group, respectively. eGFP positive cells in the R2-eGFP, R19-eGFP, and AAV2-eGFP groups were found using this imaging technique (FIGS. 96F, 96I, and 96L, respectively; see arrows). These data show that administration of a cross packaged R2-eGFP Anellovector, with Ring2 ORF1 encapsulating a Ring19 genetic element, resulted in expression of functional eGFP protein in vivo.

1. An Anellovector genetic element comprising:
a) a recombinase hybrid site;
b) one or both of an Anellovirus 5′ NCR sequence and Anellovirus 3′ NCR sequence, and
c) a sequence encoding an exogenous effector.

2. (canceled)

3. The Anellovector genetic element of claim 1, which does not comprise a vector backbone.

4. The Anellovector genetic element of claim 1, which is less than 5 kb, 4 kb, or 3 kb in length.

5. The Anellovector genetic element of claim 1, which comprises, from upstream to downstream, the recombinase hybrid site, the Anellovirus 3′ NCR sequence, the Anellovirus 5′ NCR sequence, and the sequence encoding an exogenous effector.

6. (canceled)

7. The Anellovector genetic element of claim 1, which comprises, from upstream to downstream, the Anellovirus 3′ NCR sequence, the recombinase hybrid site, the Anellovirus 5′ NCR sequence, and the sequence encoding an exogenous effector.

8.-10. (canceled)

11. The Anellovector genetic element of claim 1, wherein the recombinase hybrid site is a loxP site, an attL site, or an attR site.

12.-13. (canceled)

14. The Anellovector genetic element of claim 1, wherein the recombinase hybrid site has a nucleotide sequence of a recombinase hybrid site as listed in any of Tables V3-V5, or a sequence having at last 80%, 85%, 90%, or 95% identity thereto.

15. The Anellovector genetic element of claim 1:
(a) circular; and/or
(b) single stranded.

16.-17. (canceled)

18. The Anellovector genetic element of claim 1, which comprises an Anellovirus origin of replication.

19. (canceled)

20. A host cell comprising the Anellovector genetic element of claim 1.

21.-22. (canceled)

23. An Anellovector particle comprising the Anellovector genetic element of claim 1, encapsulated in a proteinaceous exterior.

24. The Anellovector particle of claim 23, wherein the proteinaceous exterior comprises an ORF1 molecule.

25.-28. (canceled)

29. A method of treating a disease or disorder in the subject in need thereof, the method comprising:
Administering the Anellovector particle of claim 23 to the subject,
wherein the exogenous effector is expressed in the subject,
thereby treating the disease or disorder in the subject.

30. A first DNA construct comprising, in order:
a) a first recombinase recognition site;
b) an Anellovector genetic element sequence comprising a sequence encoding an exogenous effector;
c) a second recombinase recognition site in the same orientation as the first recombinase recognition site; and
d) a vector backbone suitable for replication in a host cell;
wherein the first and second recombinase recognition sites are recognized by the same site-specific recombinase, and
wherein the DNA construct is circular and double stranded.

31.-78. (canceled)

79. A DNA construct comprising:
a) one or more of: a sequence encoding an ORF1 molecule, a sequence encoding an ORF2 molecule, and a sequence encoding an ORF3 molecule;
b) optionally a sequence encoding a replication protein; and
c) an exogenous origin.

80.-136. (canceled)

137. A method of making an Anellovector particle, the method comprising:
(i) incubating a host cell comprising the DNA construct of claim 30, under conditions suitable for expressing the ORF1 molecule; and
(ii) incubating the host cell under conditions suitable for encapsulation, within a proteinaceous exterior comprising the ORF1 molecule, of an Anellovector genetic element in the host cell.

138.-221. (canceled)

222. An anellovector comprising:
(i) a proteinaceous exterior comprising an ORF1 molecule, having an ORF1 sequence of a first Anellovirus genomic sequence, or a polypeptide comprising an amino acid sequence having at least about 70%, 75%, 80%, 85%, 90%, 95%, 96%, 97%, 98%, or 99% sequence identity thereto; and
(ii) a genetic element enclosed by the proteinaceous exterior, wherein the genetic element comprises:
a nucleic acid sequence encoding an exogenous effector; and
one or both of:
(a) an Anellovirus GC-rich region, having the sequence of an Anellovirus GC-rich region of a second Anellovirus genomic sequence, or a nucleic acid comprising a nucleotide sequence having at least about 85%, 90%, 95%, 96%, 97%, 98%, or 99% sequence identity thereto,
wherein the Anellovirus GC-rich region has a higher level of sequence similarity to the Anellovirus GC-rich region of the second Anellovirus genomic sequence than to the Anellovirus GC-rich region of the first Anellovirus genomic sequence; and
(b) an Anellovirus 5′ UTR conserved domain, having the sequence of an Anellovirus 5′ UTR conserved domain of the second Anellovirus genomic sequence, or a nucleic acid comprising a nucleotide sequence having at least about 91%, 92%, 93%, 94%, 95%, 96%, 97%, 98%, or 99% sequence identity thereto,
wherein the Anellovirus 5′ UTR conserved domain has a higher level of sequence similarity to the Anellovirus 5′ UTR conserved domain of the second Anellovirus genomic sequence than to the Anellovirus 5′ UTR conserved domain of the first Anellovirus genomic sequence.

223.-267. (canceled)

268. A system comprising a first DNA construct of claim 30 and a second DNA construct comprising:
a) one or more of: a sequence encoding an ORF1 molecule, a sequence encoding an ORF2 molecule, and a sequence encoding an ORF3 molecule;
b) optionally a sequence encoding a replication protein; and
c) an exogenous origin.

269. The system of claim 268, which further comprises a third DNA construct encoding the site-specific recombinase.

270. A composition comprising:
(I) an isolated nucleic acid molecule comprising:
(a) one or both of:
(a1) an Anellovirus GC-rich region, wherein the Anellovirus GC-rich region has a sequence of the reverse complement of the Anellovirus GC-rich region of any of SEQ ID NOs: 210, 207, or 204, or a nucleic acid sequence having at least about 90%, 95%, 96%, 97%, 98%, or 99% sequence identity thereto; and
(a2) an Anellovirus 5′ UTR conserved domain, wherein the Anellovirus 5′ UTR conserved domain has a sequence of the reverse complement of the Anellovirus 5′ UTR conserved domain of any of SEQ ID NOs: 210, 207, or 204, or a nucleic acid sequence having at least about 90%, 95%, 96%, 97%, 98%, or 99% sequence identity thereto; and
(b) a promoter element operably linked to a nucleic acid sequence encoding an exogenous effector; or
(II) an anellovector comprising:
(i) a proteinaceous exterior comprising an ORF1 molecule having an ORF1 sequence of any of SEQ ID NOs: 211, 208, or 205, or a polypeptide comprising an amino acid sequence having at least about 70%, 75%, 80%, 85%, 90%, 95%, 96%, 97%, 98%, or 99% sequence identity thereto, and
(ii) a genetic element enclosed by the proteinaceous exterior, wherein the genetic element comprises a promoter element operably linked to a nucleic acid sequence encoding an exogenous effector.
